(19) United States
(12) Patent Application Publication
(10) Pub. No.: US 2025/0051434 A1
(43) Pub. Date: Feb. 13, 2025

(54) MONOVALENT INTERLEUKIN 12 (IL-12) HETERODIMERIC FC PROTEINS

(71) Applicant: MERCK SHARP & DOHME LLC, RAHWAY, NJ (US)

(72) Inventors: Mehabaw Getahun Derebe, Burlingame, CA (US); Ghassan N. Fayad, Waban, MA (US); Veronica M. Juan, Redwood City, CA (US); Mike Lee, Palo Alto, CA (US); Ji Hea Park, San Mateo, CA (US); Piyali Saha, Dublin, CA (US); Fan Zhang, Los Altos, CA (US)

(73) Assignee: MERCK SHARP & DOHME LLC, RAHWAY, NJ (US)

(21) Appl. No.: 18/797,601

(22) Filed: Aug. 8, 2024

Related U.S. Application Data
(60) Provisional application No. 63/659,701, filed on Jun. 13, 2024.
(60) Provisional application No. 63/519,063, filed on Aug. 11, 2023.

(51) Int. Cl. C07K 16/24; A61K 39/00; A61K 45/06; A61P 35/00

(52) U.S. Cl. CPC C07K 16/244; A61K 45/06; A61P 35/00; A61K 2039/505; C07K 2317/41; C07K 2317/52; C07K 2317/71; C07K 2317/92

(57) ABSTRACT

Monovalent IL-12 heterodimeric Fc proteins that display attenuated potency at the IL-12 receptor compared to wild-type IL-12 and have an extended half-life compared to wild-type IL-12 are described. The monovalent heterodimeric IL-12 heterodimeric Fc proteins comprise a heterodimer comprising an IL-12p35 subunit first Fc domain fusion protein and an IL-p40 subunit second Fc domain fusion protein IL-12 and Fc domains wherein the first and second Fc domains comprise modifications that facilitate heterodimerization of the first Fc domain to the second Fc domain.

### CROSS-REFERENCE TO RELATED APPLICATIONS

This application claims the benefit of U.S. Provisional Patent Application Ser. No. 63/519,063 filed Aug. 11, 2023, and U.S. Provisional Patent Application Ser. No. 63/659,701 filed Jun. 13, 2024, the entire contents of both are incorporated herein by reference.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created on Jun. 26, 2024, is named 25695-US-NP_SL.xml and is 532,784 bytes in size.

### FIELD OF THE INVENTION

The present invention relates to monovalent IL-12 heterodimeric Fc proteins that display reduced potency at the IL-12 receptor compared to wild-type IL-12 and have an extended half-life compared to wild-type IL-12. In particular, the present invention relates to a monovalent IL-12 heterodimeric Fc protein comprising (i) an IL-12 p35 subunit fused to the N-terminus of a first Fc region and (ii) an IL-12 p40 subunit fused to the N-terminus of a second Fc region, wherein the first and second Fc regions are modified to form a heterodimer pair that provides a monovalent heterodimer, and at least one of the p35 subunit and the p40 subunit is modified to attenuate the IL-12 potency of the monovalent IL-12 heterodimeric Fc protein compared to wild-type IL-12.

### BACKGROUND OF THE INVENTION

IL-12 is a potent, pro-inflammatory type 1 cytokine that has long been studied as a potential immunotherapy for cancer. Unfortunately, IL-12's remarkable antitumor efficacy in preclinical models has yet to be replicated in humans. Early clinical trials in the mid-1990's showed that systemic delivery of IL-12 incurred dose-limiting toxicities. Nevertheless, IL-12's pleiotropic activity, i.e., its ability to engage multiple effector mechanisms and reverse tumor-induced immunosuppression, continues to entice cancer researchers.

The clinical success of existing therapeutic approaches involving cytokines has been limited due to off-target toxicity and pleiotropy, which is largely due to the fact that cytokines have receptors on both desired and undesired responder cells that counterbalance one another and lead to unwanted side effects. For example, in the case of IL-12, systemic administration of IL-12 leads to toxicity due to natural killer (NK)-cell mediated IFNγ production.

In recent years, cytokine engineering has emerged as a promising strategy to tailor cytokines with desired activities and reduced toxicity. There remains a need for additional approaches to improve properties of IL-12 for use as a therapeutic agent. In particular, there is a need for variants of IL-12 that can selectively activate certain downstream functions and actions over others, e.g., retain many beneficial properties of IL-12 but have reduced toxic side effects, leading to improved use as anti-cancer agents or immune modulators.

### BRIEF SUMMARY OF THE INVENTION

The present invention provides monovalent IL-12 heterodimeric Fc proteins that have an extended half-life compared to wild-type IL-12 and in particular embodiments, display reduced potency at the IL-12 receptor compared to wild-type IL-12. In particular, the present invention relates to a monovalent IL-12 heterodimeric Fc protein comprising (i) an IL-12 p35 subunit fused to the N-terminus of a first Fc region and (ii) an IL-12 p40 subunit fused to the N-terminus of a second Fc region, wherein the first and second Fc regions are modified to form a heterodimer pair that provides a monovalent heterodimer, and wherein the first and second Fc regions have been modified to provide a monovalent IL-12 heterodimeric Fc protein having a half-life that is longer than that of wild-type IL-12 or that of monovalent IL-12 heterodimeric Fc proteins without the modifications to extend half-life, and wherein at least one of the p35 subunit and the p40 subunit is modified to attenuate or reduce the potency of the monovalent IL-12 heterodimeric Fc protein.

In particular embodiments, the present invention provides a monovalent interleukin 12 (IL-12) heterodimeric Fc protein, comprising (a) an IL-12p35 subunit directly or indirectly linked to the N-terminus of a first immunoglobulin fragment crystallizable (Fc) region, and; (b) an IL-12p40 subunit directly or indirectly linked to the N-terminus of a second Fc region; wherein (i) the first Fc region and the second Fc region each comprise one or more amino acid substitutions that promote heterodimerization; (ii) at least one of the IL-12p35 and the IL-12p40 subunits of the monovalent IL-12 heterodimer Fc protein is a modified variant comprising one or more amino acid substitutions as compared to wild-type IL-12 p35 and IL-12p40 subunits, as applicable; and, (iii) the monovalent IL-12 heterodimeric Fc protein displays reduced potency compared to a monovalent IL-12 heterodimeric Fc protein comprising wild-type IL-12p35 and IL-12p40 subunits. In further embodiments, the potency is determined by measuring Signal Transducer and Activator of Transcription 4 (STAT4) protein induction in a cell-based assay comprising human embryo kidney (HEK) cells stably transfected with one or more nucleic acid molecules that express an IL-12 receptor and a STAT4-inducible reporter gene. In a further embodiment, the first Fc region and the second Fc region each comprise an CH3 domain that comprises one or more amino acid substitutions that promote heterodimerization of the first and second Fc regions.

In further embodiments of the monovalent IL-12 heterodimeric Fc, the potency is reduced at least 10-fold. In further embodiments, the monovalent IL-12 heterodimeric Fc protein of claim 1, wherein the potency is reduced at least 30-fold. In further embodiments, the monovalent IL-12 heterodimeric Fc protein of claim 1, wherein the potency is reduced from 30-fold to 200-fold.

In further embodiments of the monovalent IL-12 heterodimeric Fc, the IL-12p35 subunit includes a deletion of the N-terminal hexapeptide RNLPVA (SEQ ID NO: 238).

In further embodiments of the monovalent IL-12 heterodimeric Fc, the IL-12p35 subunit is a modified variant comprising an I41S, E44R, D159K, D159R, Y161S, Y161T, or Y161A amino acid substitution, wherein the amino acid positions correspond to amino acid positions I41, E44, D159, and Y161 as set forth in SEQ ID NO: 133.

In further embodiments of the monovalent IL-12 heterodimeric Fc, the IL-12p35 subunit is a modified variant comprising an I41S, E44R, D159K, D159R, Y161S, Y161T, or Y161A amino acid substitution amino acid substitution, wherein the amino acid positions correspond to amino acid positions I41, E44, D159, and Y161 as set forth in SEQ ID NO: 133, and wherein the IL-12p35 subunit further includes a deletion of the N-terminal hexapeptide RNLPVA (SEQ ID NO: 238).

In further embodiments of the monovalent IL-12 heterodimeric Fc, the IL-12p35 subunit is a modified variant comprising an I41S, E44R, D159K, D159R, Y161S, Y161T, or Y161A amino acid substitution amino acid substitution, wherein the amino acid positions correspond to amino acid positions I41, E44, D159, and Y161 as set forth in SEQ ID NO: 133, and the IL-12p40 subunit comprises a wild-type amino acid sequence.

In further embodiments of the monovalent IL-12 heterodimeric Fc, the IL-12p35 subunit is a modified variant comprising an I41S, E44R, D159K, D159R, Y161S, Y161T, or Y161A amino acid substitution amino acid substitution, wherein the amino acid positions correspond to amino acid positions I41, E44, D159, and Y161 as set forth in SEQ ID NO: 133, wherein the IL-12p35 subunit further comprises a deletion of the N-terminal hexapeptide RNLPVA (SEQ ID NO: 238), and the IL-12p40 subunit comprises a wild-type amino acid sequence.

In further embodiments of the monovalent IL-12 heterodimeric Fe, the IL12p40 subunit is a modified variant comprising a D87L, K104E, or K104D amino acid substitution, wherein the amino acid positions correspond to amino acid positions D87 and K104 as set forth in SEQ ID NO: 134.

In further embodiments of the monovalent IL-12 heterodimeric Fe, the IL12p40 subunit is a modified variant comprising a D87L, K104E, or K104D amino acid substitution, wherein the amino acid positions correspond to amino acid positions D87 and K104 as set forth in SEQ ID NO: 134, and the IL-12p35 subunit comprises a wild-type amino acid sequence or a wild-type amino acid sequence that further includes a deletion of the N-terminal hexapeptide RNLPVA (SEQ ID NO: 238).

In further embodiments of the monovalent IL-12 heterodimeric Fc, the IL-12p35 subunit is a modified variant comprising an I41S, E44R, D159K, D159R, Y161S, Y161T, or Y161A amino acid substitution amino acid substitution, wherein the amino acid positions correspond to amino acid positions I41, E44, D159, and Y161 as set forth in SEQ ID NO: 133, wherein the IL-12p35 subunit further includes a deletion of the N-terminal hexapeptide RNLPVA (SEQ ID NO: 238), and wherein the IL-12p40 subunit is a modified variant comprising a D87L, K104E, or K104D amino acid substitution, wherein the amino acid positions correspond to amino acid positions D87 and K104 as set forth in SEQ ID NO: 134.

In further embodiments of the monovalent IL-12 heterodimeric Fc, the IL-12p35 subunit is a modified variant comprising an I41S, E44R, D159K, D159R, Y161S, Y161T, or Y161A amino acid substitution amino acid substitution, wherein the amino acid positions correspond to amino acid positions I41, E44, D159, and Y161 as set forth in SEQ ID NO: 133, and wherein the IL-12p40 subunit is a modified variant comprising amino acid substitutions selected from P143A/P215A, P143A/P215A/K260A/R261S/V303I, P143A/P215A/K260S/R261S/V303I, K260A/R261S/V303I, K260S/R261S/V303I, K260A/R261S, and K260S/R261S, wherein the amino acid positions correspond to amino acid positions P143, P215, K260, R261, and V303 as set forth in SEQ ID NO: 134.

In further embodiments of the monovalent IL-12 heterodimeric Fc, the IL-12p35 subunit is a modified variant comprising a Y161S or Y161T amino acid substitution, wherein the amino acid position corresponds to amino acid position Y161 as set forth in SEQ ID NO: 133, and the IL-12p40 subunit comprises a wild-type amino acid sequence.

In further embodiments of the monovalent IL-12 heterodimeric Fc, the IL-12p35 subunit is a modified variant comprising a Y161S or Y161T amino acid substitution, wherein the amino acid position corresponds to amino acid position Y161 as set forth in SEQ ID NO: 133, and the IL-12p40 subunit is a modified variant comprising a D87L, K104E, or K104D amino acid substitution, wherein the amino acid positions correspond to amino acid positions D87 and K104 as set forth in SEQ ID NO: 134.

In further embodiments of the monovalent IL-12 heterodimeric Fc, the IL-12 p35 subunit is a modified variant comprising an I41S, E44R, D159K, D159R, Y161S, Y161T, or Y161A amino acid substitution amino acid substitution, wherein the amino acid positions correspond to amino acid positions an I41, E44, D159, or Y161 amino acid substitution as set forth in SEQ ID NO: 133, wherein the IL-12p35 subunit further includes a deletion of the N-terminal hexapeptide RNLPVA (SEQ ID NO: 238), and wherein the p40 subunit is a modified variant comprising amino acid substitutions selected from P143A/P215A, P143A/P215A/K260A/R261S/V303I, P143A/P215A/K260S/R261S/V303I, K260A/R261S/V303I, K260S/R261S/V303I, K260A/R261S, and K260S/R261S, wherein the amino acid positions correspond to amino acid positions P143, P215, K260, R261, and V303 as set forth in SEQ ID NO: 134.

In further embodiments of the monovalent IL-12 heterodimeric Fc, the IL-12p35 subunit is a modified variant comprising a Y161S or Y161T amino acid substitution, wherein the amino acid position corresponds to amino acid position Y161 as set forth in SEQ ID NO: 133, wherein the IL-12p35 subunit further includes a deletion of the N-terminal hexapeptide RNLPVA (SEQ ID NO: 238), and the IL-12p40 subunit comprises a wild-type amino acid sequence.

In further embodiments of the monovalent IL-12 heterodimeric Fc, the IL-12p35 subunit is a modified variant comprising a Y161S or Y161T amino acid substitution, wherein the amino acid position corresponds to amino acid position Y161 as set forth in SEQ ID NO: 133, wherein the IL-12p35 subunit further includes a deletion of the N-terminal hexapeptide RNLPVA (SEQ ID NO: 238), and the IL-12p40 subunit is a modified variant comprising a D87L, K104E, or K104D amino acid substitution, wherein the amino acid positions correspond to amino acid positions D87 and K104 as set forth in SEQ ID NO: 134.

In further embodiments of the monovalent IL-12 heterodimeric Fc, the IL-12p35 subunit is a modified variant comprising the amino acid sequence of SEQ ID NO: 140, 147, 148, 152, 153, or 154 and the IL12p40 subunit is a wild-type IL-12p40 subunit comprising the amino acid sequence set forth in SEQ ID NO: 134 or the IL-12p40 subunit is a modified variant comprising the amino acid sequence set forth in SEQ ID NO: 161, 166, 167, 192, 193, 194, 195, 196, 197, or 198.

In further embodiments of the monovalent IL-12 heterodimeric Fc, (i) the first Fc region comprises the amino acid substitutions S354C and T366W and the second Fc region comprises the amino acid substitutions Y349C, T366S, L368A, and Y407V, wherein the amino acid positions are according to the EU Index; (ii) the first Fc region comprises the amino acid substitutions Y349C, T366S, L368A, and Y407V and the second Fc region comprises the amino acid substitutions S354C and T366W, wherein the amino acid positions are according to the EU Index; (iii) the first Fc region comprises the amino acid substitutions K409W and the second Fc region comprises the amino acid substitutions D399V and F405T, wherein the amino acid positions are according to the EU Index; (iv) the first Fc region comprises the amino acid substitutions D399V and F405T and the second Fc region comprises the amino acid substitution K409W, wherein the amino acid positions are according to the EU Index; (v) the first Fc region comprises the amino acid substitution K357W and the second Fc region comprises the amino acid substitution Y349S, wherein the amino acid positions are according to the EU Index; or, (vi) the first Fc region comprises the amino acid substitution Y349S and the second Fc region comprises the amino acid substitution K357W, wherein the amino acid positions are according to the EU Index.

In further embodiments of the monovalent IL-12 heterodimeric Fc, (a)(i) the IL-12p35 subunit is a modified variant comprising an I41S, E44R, D159K, D159R, Y161S, Y161T, or Y161A amino acid substitution directly or indirectly linked to the N-terminus of a first Fc region comprising the amino acid substitutions S354C and T366W, wherein the Fc amino acid positions are according to the EU Index and the amino acid positions of the IL-12p35 subunit variant correspond to amino acid positions I41, E44, D159, or Y161 as set forth in SEQ ID NO: 133; and, (a)(ii) the IL-12p40 subunit is directly or indirectly linked to the N-terminus of a second Fc region comprising the amino acid substitutions Y349C, T366S, L368A, and Y407V, wherein the amino acid positions are according to the EU Index and the IL-12p40 subunit is wild-type; or (b)(i) the IL-12 p40 subunit is directly or indirectly linked to the N-terminus of a first Fc region comprising the amino acid substitutions S354C and T366W, wherein the amino acid positions are according to the EU Index and the IL-12p40 subunit is wild-type; and (b)(ii) the IL-12p35 subunit is a modified variant comprising an I41S, E44R, D159K, D159R, Y161S, Y161T, or Y161A amino acid substitution amino acid substitution directly or indirectly linked to the N-terminus of a second Fc region comprising the amino acid substitutions Y349C, T366S, L368A, and Y407V wherein the Fc amino acid positions are according to the EU Index and the amino acid positions of the IL-12p35 subunit variant correspond to amino acid positions I41, E44, D159, or Y161 as set forth in SEQ ID NO: 133.

In further embodiments of the monovalent IL-12 heterodimeric Fe, the first Fc region and the second Fc region each comprise one or more amino acid substitutions that increase serum half-life of the monovalent IL-12 heterodimeric Fc protein compared to the half-life of a monovalent IL-12 heterodimeric Fc protein comprising a wild-type Fc.

In further embodiments of the monovalent IL-12 heterodimeric Fe, the first Fc region and the second Fc region each comprise amino acid substitutions selected from the group consisting of (i) M252Y, S254T, and T256E; (ii) M428L and N434S; (iii) L309D, Q311H, and N434S; and (iv) H433K and N434F, wherein the amino acid positions are according to the Eu numbering scheme.

In further embodiments of the monovalent IL-12 heterodimeric Fe, the first Fc region and the second Fc region each comprise one or more amino acid substitutions that reduce an Fc region effector function.

In further embodiments of the monovalent IL-12 heterodimeric Fe, the first Fc region and the second Fc region each comprise amino acid substitutions selected from the group consisting of (i) L234A, L235A, and D265S; (ii) L234A, L235A, and P329G; (iii) L234A and L235A; (iv) H268Q, V309L, A330S, and P331S; (v) V234A, G237A, P338S, H268A, and V309L; and (vi) N297X wherein X is A, Q, or G; wherein the amino acid positions are according to the Eu numbering scheme.

In further embodiments of the monovalent IL-12 heterodimeric Fe, the IL-12p35 subunit or modified variant thereof is indirectly linked to the N-terminus of the first Fc region via a first peptide linker or non-peptidyl polymer and the wherein the IL-12p40 subunit or modified variant thereof is indirectly linked to the N-terminus of the second Fc region via a second peptide linker or non-peptidyl polymer.

In further embodiments of the monovalent IL-12 heterodimeric Fe, the first peptide linker comprises the amino acid sequence GGGGSGGGGSGGGGS (SEQ ID NO: 206) and the second peptide linker comprises the amino acid sequence GGGG (SEQ ID NO: 202).

In further embodiments of the monovalent IL-12 heterodimeric Fe, the first and second non-peptidyl polymer are independently selected from polyethylene glycol, polypropylene glycol, copolymers of ethylene glycol and propylene glycol, polyoxyethylated polyols, polyvinyl alcohol, polysaccharides, polyvinyl ethyl ether, polylactic acid, polylacticglycolic acid, lipid polymers, hyaluronic acid, and combinations thereof.

The present invention further provides a monovalent IL-12 heterodimeric Fc protein comprising (a) a first polypeptide comprising the amino acid sequence set forth in SEQ ID NO: 8 and a second polypeptide comprising the amino acid sequence set forth in SEQ ID NO: 2; (b) a first polypeptide comprising the amino acid sequence set forth in SEQ ID NO: 15 and a second polypeptide comprising the amino acid sequence set forth in SEQ ID NO: 2; (c) a first polypeptide comprising the amino acid sequence set forth in SEQ ID NO: 16 and a second polypeptide comprising the amino acid sequence set forth in SEQ ID NO: 2; (d) a first polypeptide comprising the amino acid sequence set forth in SEQ ID NO: 17 and a second polypeptide comprising the amino acid sequence set forth in SEQ ID NO: 2; (e) a first polypeptide comprising the amino acid sequence set forth in SEQ ID NO: 20 and a second polypeptide comprising the amino acid sequence set forth in SEQ ID NO: 2; (f) a first polypeptide comprising the amino acid sequence set forth in SEQ ID NO: 21 and a second polypeptide comprising the amino acid sequence set forth in SEQ ID NO: 2; or (g) a first polypeptide comprising the amino acid sequence set forth in SEQ ID NO: 22 and a second polypeptide comprising the amino acid sequence set forth in SEQ ID NO: 2.

The present invention further provides a monovalent IL-12 heterodimeric Fc protein comprising (a) a first polypeptide comprising the amino acid sequence set forth in SEQ ID NO: 74 and a second polypeptide comprising the amino acid sequence set forth in SEQ ID NO: 68; (b) a first polypeptide comprising the amino acid sequence set forth in SEQ ID NO: 81 and a second polypeptide comprising the amino acid sequence set forth in SEQ ID NO: 68; (c) a first polypeptide comprising the amino acid sequence set forth in SEQ ID NO: 82 and a second polypeptide comprising the amino acid sequence set forth in SEQ ID NO: 68; (d) a first polypeptide comprising the amino acid sequence set forth in SEQ ID NO: 83 and a second polypeptide comprising the amino acid sequence set forth in SEQ ID NO: 68; (e) a first polypeptide comprising the amino acid sequence set forth in SEQ ID NO: 86 and a second polypeptide comprising the amino acid sequence set forth in SEQ ID NO: 68; (f) a first polypeptide comprising the amino acid sequence set forth in SEQ ID NO: 87 and a second polypeptide comprising the amino acid sequence set forth in SEQ ID NO: 68; or (g) a first polypeptide comprising the amino acid sequence set forth in SEQ ID NO: 88 and a second polypeptide comprising the amino acid sequence set forth in SEQ ID NO: 68.

The present invention further provides a monovalent interleukin 12 (IL-12) heterodimeric Fc protein, comprising (i) an IL-12p35 subunit fused to the N-terminus of a first immunoglobulin fragment crystallizable (Fc) region, and; (ii) an IL-12p40 subunit fused to the N-terminus of a second Fc region; wherein the first Fc region and the second Fc region each comprise one or more amino acid substitutions that promote heterodimerization, and; wherein the IL-12p35 subunit of the monovalent IL-12 heterodimer Fc protein comprises one or more amino acid substitutions that reduce the potency of the monovalent IL-12 heterodimeric Fc protein compared to a monovalent IL-12 heterodimeric Fc protein comprising wild-type IL-12p35 and IL-12p40 subunits.

In further embodiments of the monovalent IL-12 heterodimeric Fc protein, the potency is determined by measuring Signal Transducer and Activator of Transcription 4 (STAT4) protein induction in a cell-based assay comprising human embryo kidney (HEK) cells stably transfected with one or more nucleic acid molecules that express an IL-12 receptor and a STAT4-inducible reporter gene.

In further embodiments of the monovalent IL-12 heterodimeric Fc protein, the IL-12p35 subunit comprises the amino acid sequence set forth in SEQ ID NO: 140, 147, 148, 152, 153, or 154.

In further embodiments of the monovalent IL-12 heterodimeric Fc protein, the IL-12p40 subunit comprises the amino acid sequence set forth in SEQ ID NO: 134, 161, 166, 167, 192, 193, 194, 195, 196, 197, or 198.

In further embodiments of the monovalent IL-12 heterodimeric Fc protein, the IL-12p35 subunit comprises the amino acid sequence set forth in SEQ ID NO: 140, 147, 148, 152, 153, or 154 and the IL-12p40 subunit comprises the amino acid sequence set forth in SEQ ID NO: 134, 161, 166, 167, 192, 193, 194, 195, 196, 197, or 198.

In further embodiments of the monovalent IL-12 heterodimeric Fc protein, the IL-12p35 subunit comprises the amino acid sequence set forth in SEQ ID NO: 140, 147, 148, 152, 153, or 154 and the IL-12p40 subunit comprises the amino acid sequence set forth in SEQ ID NO: 134, 192, 193, 194, 195, 196, 197, or 198.

In further embodiments of the monovalent IL-12 heterodimeric Fc protein, (a) the first Fc region comprises the amino acid sequence set forth in SEQ ID NO: 208 and the second Fc region comprises the amino acid sequence set forth in SEQ ID NO: 209; (b) the first Fc region comprises the amino acid sequence set forth in SEQ ID NO: 210 and the second Fc region comprises the amino acid sequence set forth in SEQ ID NO: 211; (c) the first Fc region comprises the amino acid sequence set forth in SEQ ID NO: 212 and the second Fc region comprises the amino acid sequence set forth in SEQ ID NO: 213; (d) the first Fc region comprises the amino acid sequence set forth in SEQ ID NO: 214 and the second Fc region comprises the amino acid sequence set forth in SEQ ID NO: 215; (e) the first Fc region comprises the amino acid sequence set forth in SEQ ID NO: 216 and the second Fc region comprises the amino acid sequence set forth in SEQ ID NO: 217; (f) the first Fc region comprises the amino acid sequence set forth in SEQ ID NO: 218 and the second Fc region comprises the amino acid sequence set forth in SEQ ID NO: 219; (g) the first Fc region comprises the amino acid sequence set forth in SEQ ID NO: 396 and the second Fc region comprises the amino acid sequence set forth in SEQ ID NO: 397; (h) the first Fc region comprises the amino acid sequence set forth in SEQ ID NO: 398 and the second Fc region comprises the amino acid sequence set forth in SEQ ID NO: 399; (i) the first Fc region comprises the amino acid sequence set forth in SEQ ID NO: 400 and the second Fc region comprises the amino acid sequence set forth in SEQ ID NO: 401; (j) the first Fc region comprises the amino acid sequence set forth in SEQ ID NO: 402 and the second Fc region comprises the amino acid sequence set forth in SEQ ID NO: 403; (k) the first Fc region comprises the amino acid sequence set forth in SEQ ID NO: 404 and the second Fc region comprises the amino acid sequence set forth in SEQ ID NO: 405; (l) the first Fc region comprises the amino acid sequence set forth in SEQ ID NO: 406 and the second Fc region comprises the amino acid sequence set forth in SEQ ID NO: 407; (m) the first Fc region comprises the amino acid sequence set forth in SEQ ID NO: 408 and the second Fc region comprises the amino acid sequence set forth in SEQ ID NO: 409; (n) the first Fc region comprises the amino acid sequence set forth in SEQ ID NO: 410 and the second Fc region comprises the amino acid sequence set forth in SEQ ID NO: 411; or (o) the first Fc region comprises the amino acid sequence set forth in SEQ ID NO: 412 and the second Fc region comprises the amino acid sequence set forth in SEQ ID NO: 413.

In further embodiments of the monovalent IL-12 heterodimeric Fc protein, (a) the first Fc region comprises the amino acid sequence set forth in SEQ ID NO: 209 and the second Fc region comprises the amino acid sequence set forth in SEQ ID NO: 208; (b) the first Fc region comprises the amino acid sequence set forth in SEQ ID NO: 211 and the second Fc region comprises the amino acid sequence set forth in SEQ ID NO: 210; (c) the first Fc region comprises the amino acid sequence set forth in SEQ ID NO: 213 and the second Fc region comprises the amino acid sequence set forth in SEQ ID NO: 212; (d) the first Fc region comprises the amino acid sequence set forth in SEQ ID NO: 215 and the second Fc region comprises the amino acid sequence set forth in SEQ ID NO: 214; (e) the first Fc region comprises the amino acid sequence set forth in SEQ ID NO: 217 and the second Fc region comprises the amino acid sequence set forth in SEQ ID NO: 216; (f) the first Fc region comprises the amino acid sequence set forth in SEQ ID NO: 219 and the second Fc region comprises the amino acid sequence set forth in SEQ ID NO: 218; (g) the first Fc region comprises the amino acid sequence set forth in SEQ ID NO: 397 and the second Fc region comprises the amino acid sequence set forth in SEQ ID NO: 396; (h) the first Fc region comprises the amino acid sequence set forth in SEQ ID NO: 399 and the second Fc region comprises the amino acid sequence set forth in SEQ ID NO: 398; (i) the first Fc region comprises the amino acid sequence set forth in SEQ ID NO: 401 and the second Fc region comprises the amino acid sequence set forth in SEQ ID NO: 400; (j) the first Fc region comprises the amino acid sequence set forth in SEQ ID NO: 403 and the second Fc region comprises the amino acid sequence set forth in SEQ ID NO: 402; (k) the first Fc region comprises the amino acid sequence set forth in SEQ ID NO: 405 and the second Fc region comprises the amino acid sequence set forth in SEQ ID NO: 404; (l) the first Fc region comprises the amino acid sequence set forth in SEQ ID NO: 407 and the second Fc region comprises the amino acid sequence set forth in SEQ ID NO: 406; (m) the first Fc region comprises the amino acid sequence set forth in SEQ ID NO: 409 and the second Fc region comprises the amino acid sequence set forth in SEQ ID NO: 408; (n) the first Fc region comprises the amino acid sequence set forth in SEQ ID NO: 411 and the second Fc region comprises the amino acid sequence set forth in SEQ ID NO: 410; or (o) the first Fc region comprises the amino acid sequence set forth in SEQ ID NO: 413 and the second Fc region comprises the amino acid sequence set forth in SEQ ID NO: 412.

In further embodiments of the monovalent IL-12 heterodimeric Fc protein, the IL-12p35 subunit is fused to the N-terminus of the first Fc region via a peptide linker comprising the amino acid sequence set forth in SEQ ID NO: 204, 205, 206, or 207; and, the IL-12p40 subunit is fused to the N-terminus of the second Fc region via a peptide linker comprising the amino acid sequence set forth in SEQ ID NO: 202.

In further embodiments of the monovalent IL-12 heterodimeric Fc protein, the IL-12p35 subunit fused to the N-terminus of the first Fc region is a first polypeptide comprising the amino acid sequence set forth in SEQ ID NO: 74, 81, 82, 83, 86, 87, or 88; and, the IL-12p40 subunit fused to the N-terminus of the second Fc region is a second polypeptide comprising the amino acid sequence set forth in SEQ ID NO: 68.

In further embodiments of the monovalent IL-12 heterodimeric Fc protein, the IL-12p35 subunit fused to the N-terminus of the first Fc region is a first polypeptide comprising the amino acid sequence set forth in SEQ ID NO: 8, 15, 16, 17, 20, 21, or 22; and, the IL-12p40 subunit fused to the N-terminus of the second Fc region is a second polypeptide comprising the amino acid sequence set forth in SEQ ID NO: 2.

The present invention further provides a method for producing a monovalent IL-12 heterodimeric Fc protein, comprising (a) providing a first nucleic acid molecule encoding a first polypeptide comprising an IL-12p35 subunit fused to the N-terminus of a first immunoglobulin fragment crystallizable (Fc) region and a second nucleic acid molecule encoding a second polypeptide comprising an IL-12p40 subunit fused to the N-terminus of a second Fc region, wherein the first Fc region and the second Fc region each comprise one or more amino acid substitutions that promote heterodimerization, and wherein the IL-12p35 subunit of the monovalent IL-12 heterodimer Fc protein comprises one or more amino acid substitutions that reduce the potency of the monovalent IL-12 heterodimeric Fc protein compared to a monovalent IL-12 heterodimeric Fc protein comprising wild-type IL-12p35 and IL-12p40 subunits; (b) transfecting a host cell with the first nucleic acid molecule and the second nucleic acid molecule to produce a transfected host cell; (c) cultivating the host cell in a culture medium under conditions for the production of the first polypeptide and the second polypeptide, which are excreted into the culture medium and associated into a monovalent IL-12 heterodimeric Fc protein comprising a first polypeptide and a second polypeptide; and (d) recovering the monovalent IL-12 heterodimeric Fc proteins from the culture medium.

In a further embodiment of the method, the first polypeptide comprises the amino acid sequence set forth in SEQ ID NO: 74, 81, 82, 83, 86, 87, or 88 and the second polypeptide comprises the amino acid sequence set forth in SEQ ID NO: 68.

In a further embodiment of the method, the first polypeptide comprises the amino acid sequence set forth in SEQ ID NO: 8, 15, 16, 17, 20, 21, or 22 and the second polypeptide comprises the amino acid sequence set forth in SEQ ID NO: 2.

In a further embodiment of the method, the first nucleic acid molecule comprises the nucleotide sequence set forth in SEQ ID NO: 223, 224, 25, 226, 227, 228, or 229 and the second nucleic acid molecule comprises the nucleotide sequence set forth in SEQ ID NO: 230.

In a further embodiment of the method, the first nucleic acid molecule comprises the nucleotide sequence set forth in SEQ ID NO: 234, 235, 236, 240, 241, 242, or 243 and the second nucleic acid molecule comprises the nucleotide sequence set forth in SEQ ID NO: 237.

The present invention further provides a polypeptide comprising the amino acid sequence set forth in SEQ ID NO: 1, 2, 3, 4, 5, 6, 7, 8, 9, 10, 11, 12 13, 14 15, 16 17, 18, 19, 20, 21, 22, 23, 24 25 26, 27, 28, 29, 30, 31, 32, 33, 34, 35 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, or 66. The present invention further provides a monovalent IL-12 heterodimeric Fc protein comprising any one of the aforementioned polypeptides.

The present invention further provides a polypeptide comprising the amino acid sequence set forth in SEQ ID NO: 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127,128, 129, 130, 131, or 132. The present invention further provides a monovalent IL-12 heterodimeric Fc protein comprising any one of the aforementioned polypeptides.

The present invention further provides a polypeptide comprising the amino acid sequence set forth in SEQ ID NO: 133, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, or 198. The present invention further provides a monovalent IL-12 heterodimeric Fc protein comprising any one of the aforementioned polypeptides.

The present invention further provides a polypeptide comprising the amino acid set forth in SEQ ID NO: 261, 262, 263, 264, 265, 266, 267, 268, 269, 270, 271, 272, 273, 274, 275, 276, 277, 278, 278, 279, 280, 281, 282, or 283. The present invention further provides a monovalent IL-12 heterodimeric Fc protein comprising any one of the aforementioned polypeptides.

The present invention further provides a polypeptide comprising the amino acid set forth in SEQ ID NO: 284, 285, 286, 287, 288, 289, 290, 291, 292, 293, 294, 295, 296, 297, 298, 299, 300, 301, 302, or 303. The present invention further provides a monovalent IL-12 heterodimeric Fc protein comprising any one of the aforementioned polypeptides.

The present invention further provides a polypeptide comprising the amino acid set forth in SEQ ID NO: 304, 305, 306, 307, 308, 309, 310, 311, 312, 313, 314, 315, 316, 317, 318, 319, 320, 321, 322, 323, 324, 325, or 326. The present invention further provides a monovalent IL-12 heterodimeric Fc protein comprising any one of the aforementioned polypeptides.

The present invention further provides a polypeptide comprising the amino acid set forth in SEQ ID NO: 327, 328, 329, 330, 331, 332, 333, 334, 335,336, 337, 338, 339, 340, 341, 342, 343, 344, 345, or 346. The present invention further provides a monovalent IL-12 heterodimeric Fc protein comprising any one of the aforementioned polypeptides.

The present invention further provides a polypeptide comprising the amino acid set forth in SEQ ID NO: 347, 348, 349, 350, 351, 352, 353, 354, 356, 357, 358, 359, 360, 361, 362, 363, 364, 365, 366, 367, 368, or 369. The present invention further provides a monovalent IL-12 heterodimeric Fc protein comprising any one of the aforementioned polypeptides.

The present invention further provides a polypeptide comprising the amino acid set forth in SEQ ID NO: 370, 371, 372, 373, 374, 375, 376, 377, 378, 379, 380, 381, 382, 383, 384, 385, 386, 387, 388, or 389. The present invention further provides a monovalent IL-12 heterodimeric Fc protein comprising any one of the aforementioned polypeptides.

The present invention further provides a nucleic acid molecule comprising the nucleotide sequence set forth in SEQ ID NO: 223, 224, 225, 226, 227, 228, 229, 230, 231, 232, 233, 249, or 250.

The present invention further provides a nucleic acid molecule comprising the nucleotide sequence set forth in SEQ ID NO: 234, 235, 236, 237, 240, 241, 242, 243, 244, 245, 246, 247, or 248.

The present invention further provides a nucleic acid molecule comprising the nucleotide sequence set forth in SEQ ID NO: 251, 252, 253, 254, 255, 256, 257, 258, 259, or 260.

The present invention further provides a pharmaceutical composition comprising any one of the monovalent IL-12 heterodimeric Fc proteins disclosed herein and a pharmaceutical carrier or diluent.

The present invention further provides for the use of any one of the monovalent IL-12 heterodimeric Fc proteins or the pharmaceutical composition disclosed herein for the manufacture of a medicament for the treatment of a cancer.

The present invention further provides any one of the monovalent IL-12 heterodimeric Fc proteins disclosed herein or the pharmaceutical composition thereof for the manufacture of a medicament for the treatment of a cancer.

The present invention further provides a method for treating a cancer comprising administering a therapeutically effective amount of any one of the monovalent IL-12 heterodimeric Fc proteins disclosed herein or the pharmaceutical composition thereof to an individual in need thereof.

The present invention further provides a therapy for treating cancer wherein a monovalent IL-12 heterodimeric Fc protein disclosed herein or the pharmaceutical composition thereof is provided in combination with an anti-cancer treatment selected from radiation treatment, chemotherapy treatment, checkpoint inhibitor treatment, antibody-drug conjugate treatment.

The present invention further provides a method for treating a cancer comprising administering a therapeutically effective amount of any one of the monovalent IL-12 heterodimeric Fc proteins disclosed herein or the pharmaceutical composition thereof and an anti-cancer treatment selected from radiation treatment, chemotherapy treatment, immune checkpoint inhibitor treatment, or antibody-drug conjugate treatment to an individual in need thereof.

### BRIEF DESCRIPTION OF THE DRAWINGS

FIG. 1 shows a cartoon representing the monovalent IL-12 heterodimeric Fc proteins of the present invention.

FIG. 2 shows the results of a HEK-Blue™ IL-12 cells potency assay of various single amino acid substituted monovalent IL-12 heterodimeric Fc proteins comprising a mutated IL-p35 subunit or mutated IL-12p40 subunit, in which the mutation results in reduced potency compared to the potency of a monovalent IL-12 heterodimeric Fc protein comprising a wild-type IL-12p35 and IL-12p40 subunits. The mutants are shown in Table 52. For each mutant, n=2.

FIG. 3 shows the results of a HEK-Blue™ IL-12 cells potency assay of double amino acid substituted monovalent IL-12 heterodimeric Fc proteins comprising a mutated IL-p35 and a mutated IL-12p40 subunit, in which the mutations result in reduced potency compared to the potency of a monovalent IL-12 heterodimeric Fc protein comprising a wild-type IL-12p35 and IL-12p40 subunits. For each mutant, n=3. Number on top of each column is the fold reduction in potency compared to monovalent IL-12 heterodimeric Fc proteins comprising wild-type IL-12p35 and IL-12p40 subunits.

FIG. 4 shows the results of a HEK-Blue™ IL-12 cells potency assay of 31BKP (IL-12-Fc-18, 30× potency reduced) and 32BKP (IL-12-Fc-19, 200× potency reduced), monovalent IL-12 heterodimeric Fc proteins comprising an IL-12p35 Y161S or IL-12p35 Y161T amino acid substitution, respectively, compared to wild-type monovalent IL-12 heterodimeric Fc proteins.

FIG. 5 shows the change in tumor size effected by IL-12-Fc-19 (200× reduced potency) and its effect on body weight over the time course of the treatment.

FIG. 6 shows the change in tumor size effected by IL-12-Fc-259 (30× reduced potency) and its effect on body weight over the time course of the treatment.

FIG. 7 shows the pharmacodynamic response of IL-12-Fc-259 (30× reduced potency) in non-human primates (NHP) over time. The y-axis represents the “IL-12-Fc-259” molecule free concentration over time.

FIG. 8 shows the pharmacokinetic response over time for IL-12-Fc-18 (30× reduced potency), IL-12-Fc-19 (200× reduced potency), and IL-12-Fc-58 (4,000× reduced potency) compared to wild-type IL-12-Fc (WT) for Q1W×2 Dosing in Rhesus Monkeys.

FIG. 9A shows the pharmacodynamic response over time for IL-12-Fc-18 (30× reduced potency) compared to wild-type IL-12-Fc (WT) for Q1W×2 Dosing in Rhesus Monkeys.

FIG. 9B shows the pharmacodynamic response over time for IL-12-Fc-19 (200× reduced potency) compared to wild-type IL-12-Fc (WT) for Q1W×2 Dosing in Rhesus Monkeys.

FIG. 9C shows the pharmacodynamic response over time for IL-12-Fc-58 (4,000× reduced potency) compared to wild-type IL-12-Fc (WT) for Q1W×2 Dosing in Rhesus Monkeys.

FIG. 10A shows a comparison of the average serum concentration of IL-12-Fc-259 sialylated to different levels in n=6 groups of mice after 14 days. Each group consisted of six mice wherein Group 1 comprised IL-12-Fc-259 with 15% sialylation, Group 2 comprised IL-12-Fc-259 with 20% sialylation, Group 3 comprised IL-12-Fc-259 with 25% sialylation, and Group 4 comprised IL-12-Fc-259 with 14.5% sialylation.

FIG. 10B shows that the rate of clearance (CL) was reduced as the degree of sialylation was increased from 14.5-15% to 20-25% for Groups 1, 2, 3, and 4 of FIG. 10A.

FIG. 11 shows an IFNγ release assay using activated Rhesus PBMCs demonstrating that sialic acid levels on attenuated IL-12-YTE-Fc as exemplified using IL-12-Fc-259 do not impact potency. Bar shows the median ±95% Confidence Interval. Each symbol represents the results using the cells of one donor.

FIG. 12 shows a representative pStat4 reporter assay performed in a HEK cell line assay for IL-12-Fc-259 having various levels of sialylation. WT IL-12-Fc is a monovalent heterodimer consisting of wt Il-12p35 linked to Fc (LALADS-KIH-Knob) and wt IL-12p40 linked to Fc (LALADS-KIH-Hole).

### DETAILED DESCRIPTION OF THE INVENTION

### I. Overview

The present invention is drawn to novel monovalent heterodimeric IL-12 heterodimeric Fc domain protein constructs, each construct comprising a heterodimer comprising an IL-12p35 subunit first Fc domain fusion protein and an IL-p40 subunit second Fc domain fusion protein IL-12 and Fc domains wherein the first and second Fc domains comprise modifications that facilitate heterodimerization of the first Fc domain to the second Fc domain. The IL-12 is composed of an α-chain (the p35 subunit; IL-12p35) and a β-chain (the p40 subunit; IL-12p40) covalently linked to form the biologically active IL-12 heterodimer. IL-12 exerts its cell signaling function through binding to a dimeric IL-12 receptor complex composed of IL-12 receptor β1 (IL-12ROI) and IL-12 receptor β2 (IL-12Rβ2) on T cells and inducing IFNγ secretion. However, the IL-12p40 subunit can also exist as a homodimer which has been reported to antagonize IL-12 activity by competing for binding to IL-12 receptor. Accordingly, the present invention addresses the short half-life of IL-12 and the potential formation of antagonistic IL12p40 homodimers by providing monovalent IL-12 heterodimeric Fc proteins as well as novel IL-12 variants with decreased potency.

### II. Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

As used herein, the term “IL-12” refers to interleukin 12. IL-12 is a heterodimeric proinflammatory cytokine encoded by two separate genes, IL-12A (IL-12p35) and IL-12B (IL-12p40). The active heterodimer (referred to as ‘p70’) comprising IL-12p35 disulfide-linked to the IL-12p40 subunit, and a homodimer of IL-12p40 are formed following protein synthesis. The IL-12p35 subunit is composed of a bundle of four alpha helices and the IL-12p40 subunit has three beta sheet domains. The mature form of the wild-type human IL-12p35 subunit comprises the amino acid sequence set forth in SEQ ID NO: 239 and the mature form of the wild-type human IL-12p40 subunit comprises the amino acid sequence set forth in SEQ ID NO: 134. The IL-12p35 subunit comprises N-glycosylation sites at amino acid positions 71-73 (amino acid sequence NES), 85-87 (amino acid sequence NGS), and 195-197 (amino acid sequence NAS) and the IL-12p40 subunit comprises N-glycosylation sites at amino acid positions 103-105 (amino acid sequence NKT), 113-115 (amino acid sequence NYS), 200-202 (amino acid sequence NYT), and 281-283 (amino acid sequence NAS). One or more of the N-glycosylation sites may N-glycosylated in various embodiments of the monovalent IL-12 heterodimeric Fc proteins of the present invention, for example, embodiments produced in mammalian cells such as CHO cells or HEK cells.

As used herein, the term “IL-12p35 subunit” refers to the mature α-chain of IL-12 and the term “IL-12p40 subunit” refers to the mature β-chain of IL-12. As discussed herein, the mature IL-12p35 subunit may comprise the wild-type human amino acid sequence or a variant of the wild-type human amino acid sequence as disclosed herein and the IL-12p40 subunit may comprise the wild-type human amino acid sequence or variant of the wild-type human amino acid sequence as disclosed herein. The monovalent IL-12 heterodimeric Fc proteins of the present invention comprise an IL-12p35 subunit and an IL-12p40 subunit with the proviso that at least one of the subunits comprises a variant amino acid sequence, either an amino acid substitution or in the case of IL-12p35, a deletion of the N-terminal hexapeptide RNLPVA (SEQ ID NO: 238), referred to herein as “Δhexapeptide”.

The IL-12 subunits of the invention, when associated together, provide a monovalent heterodimer that specifically binds to a dimeric IL-12 receptor complex comprising IL-12 receptor β1 and IL-12 receptor β2. The strength, or affinity, of specific binding can be expressed in terms of dissociation constant (KD) of the interaction, wherein a smaller KD represents greater affinity and a larger KD represents lower affinity. Binding properties can be determined by methods well known in the art such as bio-layer interferometry and surface plasmon resonance based methods, including Biacore™ and Octet™ methodologies. One such method entails measuring the rates of IL-12/IL-12 receptor complex association and dissociation, wherein rates depend on the concentration of the complex partners, the affinity of the interaction, and geometric parameters that equally influence the rate in both directions. Thus, both the association rate (ka) and the dissociation rate (kd) can be determined, and the ratio of kd/ka is equal to the dissociation constant KD (See Nature 361:186-187 (1993) and Davies et al. (1990) Annual Rev. Biochem. 59:439-473), both of which are incorporated by reference in their entirety for the methods therein.

As used herein, the term “wild type” or “WT” refers an amino acid sequence or a nucleotide sequence that is found in nature, including allelic variations. A WT protein has an amino acid sequence or a nucleotide sequence that has not been intentionally modified.

As used herein, the term “monovalent IL-12 heterodimeric Fc protein” refers to an Fc heterodimer pair in which one Fc of the Fc heterodimer pair is linked to the IL-12 p35 subunit and the other Fc of the Fc heterodimer pair is linked to the IL-12 p40 subunit, which when paired present one functional IL-12 heterodimer, thus monovalent.

The monovalent IL-12 heterodimeric Fc proteins of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated protein,” refers to a protein which is substantially free of other proteins from a cell culture such as host cell proteins. “Recombinant” means the proteins are generated using recombinant nucleic acid techniques in exogeneous host cells.

As used herein, the term “fused” or “fusion” refer to molecules in which the components (e.g., an IL-12 subunit and an Fc domain) are linked by peptide bonds, either directly or indirectly via peptide linkers, outlined herein.

As used herein, the term “peptide linker” refers to a peptide that links together two protein molecules via peptide bonds.

As used herein, the term “reduced potency” refers to having a potency reduced by at least 100% to about 400,000% at the IL-12 receptor compared to wild-type IL-12 or wild-type IL-12-Fc and, in specific embodiments, by at least 100%, 200%, 1,000%, 2,000%, 3,000%, 5,000%, 7,500%, 10,000%, 20,000%, 30,000%, 40,000%, 50,000%, 75,000%, 100,000%, 200,000%, 300,000%, or 400,000% as can be measured in an assay suitable for measuring the potency of IL-12 at its receptor. The term “reduced potency” shall mean in specific and individual embodiments a pharmacodynamic (PD) response over time of at about 2×, 5×, 10×, 20×, 30×, 50×, 75×, 100×, 200×, from 30× to 200×, 300×, 400×, 500×, 1000×, 2000×, 3000×, or 4000× reduced potency compared to wild-type IL-12-Fc. The term “reduced potency” shall mean in specific and individual embodiments a PD response over time of at 30× reduced potency, 200× reduced potency, 4000× reduced potency, or 10,000× reduced potency compared to wild-type IL-12-Fc. In specific embodiments, the potency may be determined by measuring Signal Transducer and Activator of Transcription 4 (STAT4) protein induction in a cell-based assay comprising human embryo kidney (HEK) cells stably transfected with one or more nucleic acid molecules that express an IL-12 receptor and a STAT4-inducible reporter gene.

As used herein, the term “about” in general means that the numerical value it precedes is approximate and that small variations in the numerical value would not significantly affect the practice of the disclosed embodiments. In particular embodiments, “about” refers to plus or minus up to 10% of the value it modifies (rounded up or down as applicable to the nearest whole number where appropriate for the situation, e.g., where you need a whole unit such as amino acids) and is meant to refer to a value within an acceptable error range for the particular value as determined by one skilled in the art depending in part on how the value is measured or determined. The term “about”, when modifying the quantity (e.g., mg) of a substance or composition, a parameter of a substance or composition or a parameter used in characterizing a step in a method, or the like, refers to variation in the numerical quantity that can occur. For example, such variation can occur through typical measuring, handling and sampling procedures involved in the preparation, characterization and/or use of the substance or composition, through inadvertent error in these procedures, through differences in the manufacture, source, or purity of the ingredients employed to make or use the compositions or carry out the procedures.

As used herein, the term “administration” and “treatment”, as it applies to an animal, human, experimental subject, cell, tissue, organ, or biological fluid, refers to contact of an exogenous pharmaceutical, therapeutic, diagnostic agent, or composition comprising an IL-12 variant as disclosed herein to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also mean in vitro and ex vivo treatments, e.g., of a cell, by a reagent, diagnostic, binding compound, or by another cell. The term “subject” includes any organism, preferably an animal, more preferably a mammal (e.g., human, rat, mouse, dog, cat, rabbit). In a preferred embodiment, the term “subject” refers to a human.

As used herein, the term “amino acid” refers to a simple organic compound containing both a carboxyl (—COOH) and an amino (—NH₂) group. Amino acids are the building blocks for proteins, polypeptides, and peptides. Amino acids occur in L-form and D-form, with the L-form in naturally occurring proteins, polypeptides, and peptides. Amino acids and their code names are set forth in the following Table 1.

TABLE 1

| | Three letter | One letter |
|---|---|---|
| Amino acid | code | code |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein, the term “one or more amino acid substitutions” shall refer to at least one amino acid substitution. The term “one or more amino acid substitutions” shall mean in specific and individual embodiments 1-10, 1-5, 1-4, 1-3, 1-2 or 1 amino acid substitutions. The term “one or more amino acid substitutions” shall mean in specific and individual embodiments one or more substitutions up to 10%, 5%, 4%, 3%, 2%, or 1%, rounded up or down as applicable to the nearest whole number.

As used herein, where a dash (“/”) is used between amino acids, it is intended to refer to all recited amino acid substitutions (e.g., in “P143A/P215A”, both P143A and P215A substitutions are intended; or in K260S/R261S/V303I, all 3 substitutions K260S, R261S, and V303I are intended).

As used herein, the symbol delta (“Δ”) refers to a deletion of what follows. For example, the symbol “Δ” before a gene/region means a deletion of that sequence/region and the symbol “Δ” before hexapeptide means a deletion of the hexapeptide.

As used herein, the term “antibody” or “immunoglobulin” refers to a glycoprotein comprising at least two heavy chains (HCs) and two light chains (LCs) inter-connected by disulfide bonds. Each HC is comprised of a heavy chain variable region or domain (V_{H}) and a heavy chain constant region or domain. Each light chain is comprised of an LC variable region or domain (V_{L}) and a LC constant domain. In certain naturally occurring IgG, IgD, IgE, IgM, and IgA antibodies, the heavy chain constant region is comprised of three domains, CH1, CH2 and CH3. In general, the basic antibody structural unit for antibodies is a Y-shaped tetramer comprising two HC/LC pairs (2H). Each tetramer includes two identical pairs of polypeptide chains, each pair having one LC (about 25 kDa) and HC chain (about 50-70 kDa) (H+L). Each HC:LC pair comprises one V_{H}:one V_{L} pair. The one V_{H}:one V_{L} pair may be referred to by the term “Fab”. Thus, each antibody tetramer comprises two Fabs, one per each arm of the Y-shaped antibody. See Béranger, et al., Ed. Ginetoux, Correspondence between the IMGT unique numbering for C-DOMAIN, the IMGT exon numbering, the Eu and Kabat numberings: Human IGHG, created: 17/05/2001, Version: 08/06/2016, which is accessible at www.imgt.org/IMGTScientificChart/Numbering/Hu_IGHGnber.html).

The constant regions of the antibodies may mediate the binding of the immunoglobulin to host tissues or factors, including various cells of the immune system (e.g., effector cells) and the first component (C1q) of the classical complement system. Typically, the numbering of the amino acids in the heavy chain constant domain begins with number 118, which is in accordance with the Eu numbering scheme. The Eu numbering scheme is based upon the amino acid sequence of human IgG1 (Eu), which has a constant domain that begins at amino acid position 118 of the amino acid sequence of the IgG1 described in Edelman et al., Proc. Natl. Acad. Sci. USA. 63: 78-85 (1969), and is shown for the IgG1, IgG2, IgG3, and IgG4 constant domains in Béranger et al., op. cit.

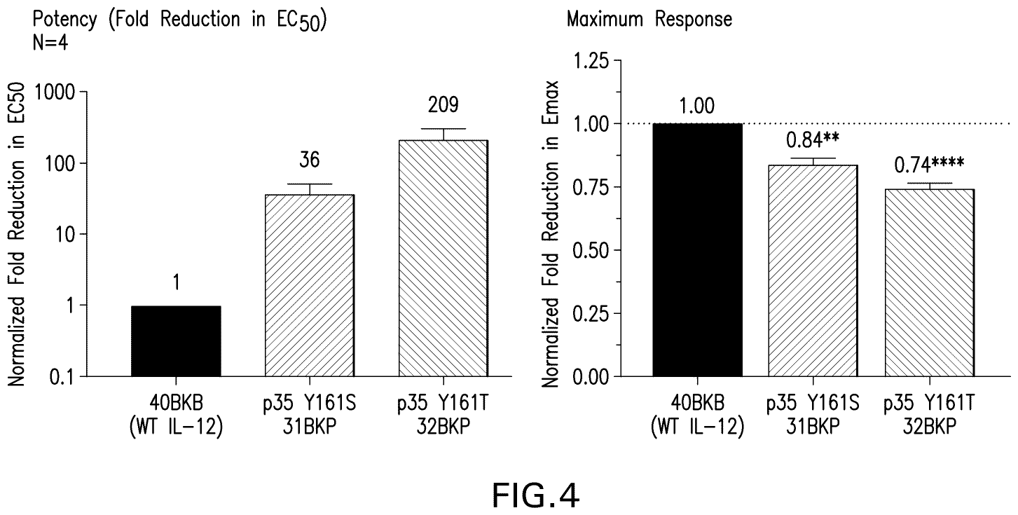

As used herein, the term “ablate” refers to a decrease or removal of binding and/or activity. Thus for example, “ablating FcγR binding” means the Fc region amino acid variant has less than 50% starting binding as compared to an Fc region not containing the specific variant, with less than 70-80-90-95-98% loss of binding being preferred, and in general, with the binding being below the level of detectable binding in a Surface Plasmon Resonance assay. Of particular use in the ablation of FcγR binding are those shown in FIG. 4. However, unless otherwise noted, the Fc monomers of the invention retain binding to the FcRn.

As used herein, the term “ADCC” or “antibody dependent cell-mediated cytotoxicity” refers to the cell-mediated reaction wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity. As is discussed herein, many embodiments of the invention ablate ADCC activity entirely.

As used herein, the term “modification” refers to an amino acid substitution, insertion, and/or deletion in a polypeptide sequence or an alteration to a moiety chemically linked to a protein. For example, a modification may be an altered carbohydrate or PEG structure attached to a protein.

As used herein, the term “amino acid modification” refers to an amino acid substitution, insertion, and/or deletion in a polypeptide sequence. For clarity, unless otherwise noted, the amino acid modification is always to an amino acid encoded by a nucleic acid, e.g., the naturally occurring amino acids or an amino acid encoded in an orthogonal translation system.

As used herein, the term “amino acid substitution” or “substitution” refers to the replacement of an amino acid at a particular position in a parent polypeptide sequence with a different amino acid. In particular, in some embodiments, the substitution is with a natural or unnatural amino acid that does not ordinarily occur at the particular position. For example, the substitution Y261S or 261S refers to a variant polypeptide, in this case a p35 subunit variant, in which the tyrosine acid at position 261 is replaced with serine. For clarity, a protein which has been engineered to change the nucleic acid coding sequence but not to change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

As used herein, the term “variant protein”, “protein variant”, or “variant” refers to a protein that differs from that of a parent protein by virtue of at least one modification. Protein variant may refer to the protein itself, a composition comprising the protein, the amino acid sequence that encodes it, or the DNA sequence that encodes it. The modification can be an addition, deletion, or substitution. As described below, in some embodiments the parent polypeptide, for example an Fc parent polypeptide, is a human wild-type sequence, such as the Fc region from IgG1, IgG2, IgG3 or IgG4. The protein variant sequence herein will preferably possess at least about 80% identity with a parent protein sequence, and in specific and individual embodiments at least about 85%, 90%, 91%, 92%, 93%, 94%, 95%, 96%, 97%, 98% or 99% identity.

As used herein, the term “protein” refers to at least two covalently attached amino acids, which includes proteins, polypeptides, oligopeptides and peptides. When a biologically functional unit comprises two or more proteins, each protein may be referred to as a “monomer” or as a “subunit” or as a “domain”; and the biologically functional molecule may be referred to as a “complex”. The two or more proteins of a complex may be covalently attached to each other, e.g., attached by one or more disulfide linkages, or non-covalently attached to each other, e.g., electrostatic or hydrophobic interactions. In some embodiments, the term “monomer” refers to a polypeptide or protein comprising one or more components, fragments, or subunits of a protein(s), and the components, fragments, or subunits are covalently attached.

As used herein, the term “residue” refers to an amino acid position in a protein and its associated amino acid identity. For example, Asparagine 297 (also referred to as Asn297 or N297) is a residue at EU position 297 in the Fc domain of the human antibody IgG1 (position numbering is according to the EU numbering scheme, i.e., EU index).

As used herein, the term “IgG subclass modification” or “isotype modification” refers to an amino acid modification that converts one amino acid of one IgG isotype to the corresponding amino acid in a different, aligned IgG isotype. For example, because IgG1 comprises a tyrosine at EU position 296 and IgG2 a phenylalanine at EU position 296, a F296Y substitution in IgG2 is considered an IgG subclass modification.

As used herein, the term “immunoglobulin (Ig) domain” refers to a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-215 according to the EU index. “Hinge” refers to positions 216-230 according to the EU index. “CH2” refers to positions 231-340 according to the EU index, and “CH3” refers to positions 341-447 according to the EU index. The dimerization of the CH3 domain in the constant region of the heavy chain plays a pivotal role in the assembly of an antibody. As shown in Table 2, the exact numbering and placement of the heavy chain domains can be different among different numbering systems.

TABLE 2

| | Eu Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

As used herein, the term “hinge” or “hinge region” or “antibody hinge region” or “immunoglobulin hinge region” refers to the flexible polypeptide comprising the amino acids between the first and second heavy chain constant domains of an antibody. Structurally, the IgG CHI domain ends at EU position 215, and the IgG CH2 domain begins at residue EU position 231. Thus for IgG, the antibody hinge is herein defined to include positions 216 (E216 in IgG1) to 230 (P230 in IgG11, wherein the numbering is according to the EU index. In some embodiments, for example in the context of an Fc region, the hinge is included, generally referring to positions 216-230.

As used herein, the term “effector function” refers to a biochemical event that results from the interaction of an antibody Fc region with an Fc receptor or ligand. Effector functions include but are not limited to ADCC, Antibody-dependent cellular phagocytosis (ADCP), and cell-dependent cytotoxicity (CDC).

As used herein, the term “IgG Fc ligand” or “Fc ligand” refers to a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, Clq, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors.

As used herein, the term “Fc gamma receptor”, “FcγR” refers to any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

As used herein, the term “FcRn” or “neonatal Fc receptor” refers to a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin (B2-microglobulin) and the heavy chain is encoded by the FcRn gene. Unless otherwise noted herein, FcRn or an FcRn protein refers to the complex of FcRn heavy chain with B2-microglobulin. A variety of Fc variants can be used to increase binding to the FcRn, and in some cases, to increase serum half-life. In general, unless otherwise noted, the Fc monomers of the invention retain binding to the FcRn (and, as noted below, can include amino acid variants to increase binding to the FcRn).

As used herein, the term “parent polypeptide” or “parent protein” refers to a starting polypeptide or protein that is subsequently modified to generate a variant. The parent polypeptide may be a naturally occurring polypeptide (wild-type), or a variant or engineered version of a naturally occurring polypeptide. Parent polypeptide may refer to the polypeptide itself, compositions that comprise the parent polypeptide, or the amino acid sequence that encodes it.

As used herein, the term “Fc” or “Fc region” or “Fc domain” refers to the polypeptide comprising the constant region of an antibody, in some instances, excluding all of the first constant region immunoglobulin domain (e.g., CHI) or a portion thereof, and in some cases, optionally including all or part of the hinge. For IgG, the Fc domain comprise immunoglobulin domains CH2 and CH3 (C72 and C73), and optionally all or a portion of the hinge region between CH1 (C71) and CH2 (C72). Thus, in some cases, the Fc domain includes, from N- to C-terminal, CH2-CH3 and hinge-CH2-CH3. In some embodiments, the Fc domain is that from IgG1, IgG2, IgG3 or IgG4, with IgG1 hinge-CH2-CH3 and IgG4 hinge-CH2-CH3 finding particular use in many embodiments. Additionally, in the case of human IgG1 Fc domains, frequently the hinge includes a C220S amino acid substitution. Furthermore, in the case of human IgG4 Fc domains, frequently the hinge includes a S228P amino acid substitution. Although the boundaries of the Fc region may vary, the human IgG heavy chain Fc region is usually defined to include residues E216, C226, or A231 to its carboxyl-terminus, wherein the numbering is according to the EU index. In some embodiments, as is more fully described below, amino acid modifications are made to the Fc region, for example to alter binding to one or more FcγR or to the FcRn. The IgG1 heavy chain Fc region comprises an N-glycosylation site at amino acid positions 297-299 (amino acid sequence NST; position numbers according to Eu numbering), which may be glycosylated in various embodiments of the monovalent IL-12 heterodimeric Fc proteins of the present invention, in particular, embodiments produced in mammalian cells such as CHO cells or HEK cells.

As will be appreciated by those in the art, the exact numbering and placement of the heavy constant region domains can be different among different numbering systems (See Table 2). A useful comparison of heavy constant region numbering according to EU and Kabat is as below, see Edelman et al., 1969, Proc. Natl. Acad. Sci. USA 63:78-85 and Kabat et al., 1991, Sequences of Proteins of Immunological Interest, 5th Ed., United States Public Health Service, National Institutes of Health, Bethesda, entirely incorporated by reference.

As used herein, the term “Fc variant” or “variant Fc” refers to a protein comprising an amino acid modification in an Fc domain. The modification can be an addition, deletion, or substitution. The Fc variants of the present invention are defined according to the amino acid modifications that compose them. Thus, for example, L235A or 235A is an Fc variant with the substitution for alanine at position 235 relative to the parent Fc polypeptide, wherein the numbering is according to the EU index. Likewise, M252Y/S254T/T256E (“YTE”) defines an Fc variant with the substitutions M252Y, S254T, and T256E relative to the parent Fc polypeptide. For all positions discussed in the present invention that relate to Fc, unless otherwise noted, the amino acid position numbering is according to the EU index. The EU index or EU numbering scheme refers to the numbering of the EU antibody (Edelman et al., 1969, Proc. Natl. Acad. Sci. USA 63:78-85, hereby incorporated by reference).

As used herein, the term “fusion protein” refers to a covalent joining of at least two proteins or protein domains by an amide bond. Fusion proteins may comprise artificial sequences, e.g., a peptide linker, variant Fc domains, a variant IL-12p40 subunit domain, a variant IL-12p35 subunit domain, etc. as described herein. By “Fc fusion protein” or “immunoadhesin” herein is meant a protein comprising an Fc region, generally linked directly or indirectly, as described herein) to one or more different protein domains. Accordingly, an “IL-12 Fc fusion” comprises an Fc region directly or indirectly linked to an IL-12p40 subunit, an IL12p35 subunit and/or single-chain IL-12 complex (scIL-12), as described herein. In some instances, two Fc fusion proteins can form a homodimeric Fc fusion protein or a heterodimeric Fc fusion protein.

As used herein, the term “position” refers to a location in the sequence of a protein. Positions may be numbered sequentially, or according to an established format, for example the EU index for Fc numbering.

As used herein, the term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include non-expressed DNA segments that, for example, form recognition sequences for other proteins. “Genes” can be obtained from a variety of sources, including cloning from a source of interest or synthesizing from known or predicted sequence information, and may include sequences designed to have desired parameters. Genes include both naturally occurring nucleotide sequences encoding a molecule of interest and synthetically derived nucleotide sequences encoding a molecule of interest, for example, complementary DNA (cDNA) obtained from a messenger RNA (mRNA) nucleotide sequence.

As used herein, the term “polynucleotides” discussed herein form part of the present invention. A “polynucleotide”, “nucleic acid” or “nucleic acid molecule” include DNA and RNA, single- or double-stranded. Polynucleotides e.g., encoding an immunoglobulin chain or component of the antibody display system of the present invention, may, in an embodiment of the invention, be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like.

Polynucleotides e.g., encoding IL-12 variants of the present invention, may be operably associated with a promoter. A “promoter” or “promoter sequence” is, in an embodiment of the invention, a DNA regulatory region capable of binding an RNA polymerase in a cell (e.g., directly or through other promoter-bound proteins or substances) and initiating transcription of a coding sequence. A promoter sequence is, in general, bounded at its 3′ terminus by the transcription initiation site and extends upstream (5′ direction) to include the minimum number of bases or elements necessary to initiate transcription at any level. Within the promoter sequence may be found a transcription initiation site (conveniently defined, for example, by mapping with nuclease S1), as well as protein binding domains (consensus sequences) responsible for the binding of RNA polymerase. The promoter may be operably associated with other expression control sequences, including enhancer and repressor sequences or with a nucleic acid of the invention. Promoters which may be used to control gene expression include, but are not limited to, cytomegalovirus (CMV) promoter (U.S. Pat. Nos. 5,385,839 and 5,168,062), the SV40 early promoter region (Benoist, et al., Nature 290: 304-310 (1981)), the promoter contained in the 3′ long terminal repeat of Rous sarcoma virus (Yamamoto et al., Cell 22: 787-797 (1980)), the herpes thymidine kinase promoter (Wagner et al., Proc. Natl. Acad. Sci. USA 78: 1441-1445 (1981)), the regulatory sequences of the metallothionein gene (Brinster et al., Nature 296: 39-42 (1982)); prokaryotic expression vectors such as the β-lactamase promoter (Villa-Komaroff et al., Proc. Natl. Acad. Sci. USA 75: 3727-3731 (1978)), or the tac promoter (DeBoer et al., Proc. Natl. Acad. Sci. USA 80: 21-25 (1983)); see also “Useful proteins from recombinant bacteria” in Scientific American 242: 74-94 (1980); and promoter elements from yeast or other fungi such as the Gal 4 promoter, the ADC (alcohol dehydrogenase) promoter, PGK (phosphoglycerol kinase) promoter or the alkaline phosphatase promoter.

As used herein, the terms “vector”, “cloning vector” and “expression vector” include a vehicle (e.g., a plasmid) by which a DNA or RNA sequence can be introduced into a host cell so as to transform the host and, optionally, promote expression and/or replication of the introduced sequence. Polynucleotides encoding an IL-12 variant of the present invention may, in an embodiment of the invention, be in a vector.

As used herein, the terms “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny of a parent cell will have precisely identical DNA content, due to deliberate or inadvertent mutations. Mutant progeny having the same function or biological activity as screened for in the originally transformed cell are included. Where distinct designations are intended, it will be clear from the context.

As used herein, the term “control sequences” or “regulatory sequences” refers to DNA sequences necessary for the expression of an operably linked coding sequence in a particular host organism. The control sequences that are suitable for expression in eukaryotes, for example, include a promoter, operator or enhancer sequences, transcription termination sequences, and polyadenylation sequences for expression of a messenger RNA encoding a protein and a ribosome binding site for facilitating translation of the messenger RNA.

As used herein, a nucleic acid is “operably linked” when it is placed into a functional relationship with another nucleic acid sequence, e.g., a regulatory sequence. For example, DNA for a pre-sequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “expression” as used herein is defined as the transcription and/or translation of a particular nucleotide sequence.

As used herein, the term “treat” or “treating” means to administer a therapeutic moiety, such as a composition containing any of the monovalent IL-12 heterodimeric Fc proteins of the present invention, internally or externally to a subject or patient having one or more disease symptoms, or being suspected of having a disease, for which the therapeutic moiety has therapeutic activity. In specific embodiments, the monovalent IL-12 heterodimeric Fc proteins can be administered topically, subcutaneously, intramuscular, intradermally, intravenously, or systemically. Typically, the moiety is administered in an amount effective to: (i) alleviate one or more disease symptoms in the treated subject or population, whether by inducing the regression of or inhibiting the progression of such symptom(s) by any clinically measurable degree, or (ii) inhibiting or reducing the severity of the disease in an individual. The amount of a therapeutic moiety that is effective to alleviate any particular disease symptom, and/or to inhibit or reduce the severity of the disease, including in specific embodiments cancer or proliferative disease, in the individual may vary according to factors such as the injury or disease state, age, and/or weight of the individual, and the ability of the therapeutic moiety to elicit a desired response in the individual. Whether one or more disease symptoms have been alleviated or the severity of the disease inhibited or reduced can be assessed by any clinical measurement typically used by physicians or other skilled healthcare providers to assess the severity or progression status of the symptom(s) or disease. Thus, the terms denote that a beneficial result has been or will be conferred on a human or animal individual exhibiting a disease symptom or diagnosed as having a disease (“in need thereof”). Treatment with monovalent IL-12 heterodimeric Fc proteins could also be combined with other interventions, treatments, moieties or agents (in specific embodiments, antibodies, nucleic acids, vaccines and small molecule compounds, radiation) to treat other symptoms or diseases.

As used herein, the term “therapeutically effective amount” refers to a quantity of a specific substance sufficient to achieve a desired effect in an individual being treated. For instance, this may be the amount necessary to alleviate any particular disease symptom or inhibit or reduce the severity of a disease in an individual.

As used herein, the term “combination therapy” refers to treatment of a human or animal individual comprising administering a first therapeutic agent and a second therapeutic agent consecutively or concurrently to the individual. In general, the first and second therapeutic agents are administered to the individual separately and not as a mixture; however, there may be embodiments where the first and second therapeutic agents are mixed prior to administration. In this application, “agent” and “moiety” are used interchangeably.

As used herein, the term “solvate” means a physical association of monovalent IL-12 heterodimeric Fc protein disclosed herein with one or more solvent molecules. This physical association involves varying degrees of ionic and covalent bonding, including hydrogen bonding.

In certain instances, the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more monovalent IL-12 heterodimeric Fc proteins disclosed herein may optionally be converted to a solvate. Preparation of solvates is generally known. Thus, for example, M. Caira et al., J. Pharma. Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al., AAPS PharmSciTechours., 5(1), article 12 (2004); and A. L. Bingham et al., Chem. Commun., 603-604 (2001). A typical, non-limiting, process involves dissolving the monovalent IL-12 heterodimeric Fc protein in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

As used herein, the term “pharmaceutically acceptable salt” includes acid addition salts and basic salts.

Exemplary acid addition salts include acetates, ammonium, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (also known as mesylates), naphthalenesulfonates, nitrates, oxalates, phosphates, propionates, salicylates, succinates, sulfates, tartarates, thiocyanates, toluenesulfonates (also known as tosylates), and the like. Additionally, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. 2^{nd} Revised Ed. (2011) Zurich: Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference thereto. In one embodiment, an acid salt is an ammonium salt or a di-ammonium salt.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and basic salts are intended to be pharmaceutically acceptable salts within the scope of the present disclosure and all acid and basic salts are considered equivalent to the free forms of the corresponding compounds for purposes of the present disclosure.

As used herein, the terms “N-glycan” and “glycoform” are used interchangeably and refer to an N-linked oligosaccharide, for example, one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. The predominant sugars found on glycoproteins are glucose (Glc), galactose (Gal), mannose (Man), fucose (Fuc), N-acetylgalactosamine (GalNAc), N-acetylglucosamine (GlcNAc) and sialic acid (Sia). Sialic acids are a class of α-keto acid sugars with a nine-carbon backbone. The most common member of this group is N-acetylneuraminic acid (Neu5Ac or NANA) found in animals and some prokaryotes. The processing of the sugar groups occurs co-translationally in the lumen of the endoplasmic reticulum (ER) and continues post-translationally in the Golgi apparatus for N-linked glycoproteins.

N-glycans have a common pentasaccharide core of Man₃GlcNAc₂ comprising a mannose linked at its reducing end to the nonreducing end of a chitobiose core (GlcNAcβ1-4GlcNAc) in a β1,4 linkage and one mannose residue linked to the β1,4 linked mannose in an α1,3 linkage and the other mannose linked to the β1,4-linked mannose in an α1,6 linkage, represented by the structure showing the Man₃GlcNAc₂ linked to an asparagine residue comprising an N-glycosylation site in a glycoprotein

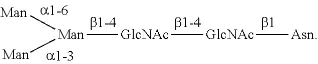

The GlcNAc residue at the reducing end may also be linked to a fucose residue in an α1,6 linkage. Usually, N-glycan structures are presented with the nonreducing end to the left and the reducing end to the right or the nonreducing end at the top and the reducing end at the bottom. The reducing end of the N-glycan is the end that is attached to the Asn residue comprising the glycosylation site on the protein. N-glycans differ with respect to the number of branches (antennae) comprising peripheral sugars (e.g., GlcNAc, galactose, fucose and sialic acid) that are added to the Man₃GlcNAc₂ (“Man₃”) core structure, which is also referred to as the “trimannose core”, the “pentasaccharide core”, the trimannosyl core”, or the “paucimannose core”.

N-glycans are classified according to their branched constituents (e.g., high mannose, complex, or hybrid). A “high mannose” type N-glycan comprises five or more mannose residues. A “complex” type N-glycan typically has at least one GlcNAc residue attached in a β1,2 linkage to the nonreducing end of the mannose residue at the nonreducing end of the 1,3 mannose arm and at least one GlcNAc residue attached in a β1,2 linkage to the nonreducing end of the mannose residue at the nonreducing end of the 1,6 mannose arm of the trimannose core. Complex N-glycans may further include galactose (“Gal”) or N-acetylgalactosamine (“GalNAc”) residues that are optionally further linked to sialic acid (“Sia”) or Sia derivatives (e.g., “NANA” or “NeuAc”, where “Neu” refers to neuraminic acid and “Ac” refers to acetyl). Complex N-glycans may also have intrachain substitutions comprising “bisecting” GlcNAc residue and core fucose (“Fuc”) residue. Complex N-glycans may also have multiple antennae on the “trimannose core,” often referred to as “multiantennary N-glycans.” A “hybrid” N-glycan comprises at least one GlcNAc attached in a β1,2 linkage to the nonreducing end of the mannose residue at the nonreducing end of the 1,3 mannose arm of the trimannose core and zero or more mannoses attached to the nonreducing end of the mannose on the nonreducing end of the 1,6 mannose arm of the trimannose core. The GlcNAc residue may then be attached to a galactose residue and the galactose residue may be attached to a sialic acid residues. The various N-glycans are also referred to as “glycoforms.”

With respect to complex N-glycans, the terms “G-2”, “G-1”, “G0”, “G1”, “G2”, “A1”, and “A2” mean the following. “G-2” refers to an N-glycan structure that can be characterized as Man₃GlcNAc₂; the term “G-1” refers to an N-glycan structure that can be characterized as GlcNAcMan₃GlcNAc₂; the term “G0” refers to an N-glycan structure that can be characterized as GlcNAc₂Man₃GlcNAc₂; the term “G1” refers to an N-glycan structure that can be characterized as GalGlcNAc₂Man₃GlcNAc₂; the term “G2” refers to an N-glycan structure that can be characterized as Gal₂GlcNAc₂Man₃GlcNAc₂; the term “A1” refers to an N-glycan structure that can be characterized as SiaGal₂GlcNAc₂Man₃GlcNAc₂; and, the term “A2” refers to an N-glycan structure that can be characterized as Sia₂Gal₂GlcNAc₂Man₃GlcNAc₂. Unless otherwise indicated, the terms “G-2”, “G-1”, “G0”, “G1”, “G2”, “A1”, and “A2” refer to N-glycan species that lack fucose attached to the GlcNAc residue at the reducing end of the N-glycan. When the term includes an “F”, the “F” indicates that the N-glycan species contains a fucose residue on the GlcNAc residue at the reducing end of the N-glycan. For example, G0F, G1F, G2F, A1F, and A2F all indicate that the N-glycan further includes a fucose residue attached to the GlcNAc residue at the reducing end of the N-glycan. Lower eukaryotes such as yeast and filamentous fungi do not normally produce N-glycans that produce fucose.

Complex N-glycans include biantennary N-glycans, bisected N-glycans, and multiantennary N-glycans. With respect to a biantennary N-glycan, the N-glycan comprises a 1,6 mannose arm and a 1,3 mannose arm in which the 1,6 mannose arm and the 1,3 mannose arm each comprises one GlcNAc residue linked in a β1,2 linkage to the mannose residue at the non-reducing end of the arm. Each GlcNAc residue may be independently further attached to a galactose residue and each galactose residue may be independently further attached to a sialic acid residue. Thus, biantennary N-glycans may include N-glycans having the short-hand formula GlcNAc₂Man₃GlcNAc₂, Gal₍₁₋₂₎GlcNAc₂Man₃GlcNAc₂, or Sia₍₁₋₂₎Gal₍₁₋₂₎GlcNAc₂Man₃GlcNAc₂. The term “(1-2)” refers to 1 or 2 sugar residues.

With respect to multiantennary N-glycans, the term “multiantennary N-glycan” refers to a biantennary N-glycan that further comprises (i) a GlcNAc residue attached in a β1,4 linkage to the nonreducing end of the mannose residue comprising the non-reducing end of the 1,6 arm or the 1,3 arm of the N-glycan or (ii) a GlcNAc residue is attached in a β1,4 linkage to the nonreducing end of the mannose residue comprising the non-reducing end of the 1,6 arm and a GlcNAc residue is attached in a β1,4 linkage to the nonreducing end of the mannose residue comprising the non-reducing end of the 1,3 arm of the N-glycan. Each GlcNAc residue may be independently further attached to a galactose residue and the galactose residue may be independently further attached to a sialic acid residue. Thus, multiantennary N-glycans can be characterized by the short-hand formulas GlcNAc₍₃₋₄₎Man₃GlcNAc₂, Gal₍₁₋₄₎GlcNAc₍₃₋₄₎Man₃GlcNAc₂, or Sia₍₁₋₄₎Gal₍₁₋₄₎GlcNAc₍₃₋₄₎Man₃GlcNAc₂. The term “(1-4)” refers to 1, 2, 3, or 4 residues and the term “(3-4)” refers to 3 or 4 sugar residues.

With respect to bisected N-glycans, the term “bisected N-glycan” refers to N-glycans in which the reducing end of a GlcNAc residue is linked in a β1,4 linkage to the nonreducing end of the central mannose residue at the nonreducing end of the trimannose core. A bisected N-glycan may be characterized by the formula GlcNAc₃Man₃GlcNAc₂ wherein each mannose residue is linked at its non-reducing end to a GlcNAc residue. In contrast, when a multiantennary N-glycan is characterized as GlcNAc₃Man₃GlcNAc₂, the short-hand formula indicates that two GlcNAc residues are linked to the mannose residue at the non-reducing end of one of the two arms of the N-glycan and one GlcNAc residue is linked to the mannose residue at the non-reducing end of the other arm of the N-glycan. While the GlcNAc residues at the end of the 1,3 and 1,6 arms of the bisected N-glycan may each be further linked to a galactose residue, which may be further linked to a sialic acid residue, the bisecting GlcNAc residue is not further extended.

In particular embodiments of the present invention, compositions of the monovalent IL-12 heterodimeric Fc proteins of the present invention may comprise a heterogenous population of N-glycans selected from high mannose N-glycans, complex N-glycans, and hybrid N-glycans.

### III. Monovalent IL-12 Heterodimeric Fc Proteins

Interleukin 12 (IL-12) is an attractive oncology immunotherapy candidate due to its anti-tumor activities on both the innate (NK cells) and adaptive (T cells) immunity. While encouraging anti-tumor activities in animal models have been observed, systemic administration of native IL-12 showed modest anti-tumor effects in humans due in part to severe toxicity limiting the administered dose. Recent clinical data with localized delivery of IL-12 suggest a path forward for IL-12 if its therapeutic window could be increased.

More recently, a long half-life antibody-IL-12 fusion protein showed that significantly higher doses could be tolerated compared to native IL-12. The present invention is intended to increase therapeutic window of IL-12 through using a combination of Fc fusion and potency reduction, resulting in a sustained pharmacodynamic (PD) profile and allowing less frequent systemic administration.

We first generated a library of monovalent IL-12 heterodimeric Fc proteins comprising one or more amino acid substitutions in the p35 subunit and/or the p40 subunit and identified those mutants that conferred reduced potency potential via structure-based engineering. The extent of the potency reduction was further tested experimentally in vitro in an engineered cell line, primary human immune cells, and primary rhesus monkey immune cells. Although the potency of the IL-12 mutants are reduced, they are still capable of reducing tumor size in an in vivo mouse tumor model similar to wild-type IL-12. Furthermore, potency reduced mutants extended the half-life of IL-12-Fc by 10-fold in rhesus monkeys while remaining capable of driving sustained pharmacodynamic responses. In particular embodiments, the potency of the monovalent IL-12 heterodimeric Fc protein is reduced at least 2-fold. In particular embodiments, the potency of the monovalent IL-12 heterodimeric Fc protein is reduced at least 5-fold. In particular embodiments, the potency of the monovalent IL-12 heterodimeric Fc protein is reduced at least 10-fold. In particular embodiments, the potency of the monovalent IL-12 heterodimeric Fc protein is reduced at least 20-fold. In particular embodiments, the potency of the monovalent IL-12 heterodimeric Fc protein is reduced at least 30-fold. In a further embodiment, the potency of the monovalent IL-12 heterodimeric Fc protein is reduced between 30-fold and 200-fold.

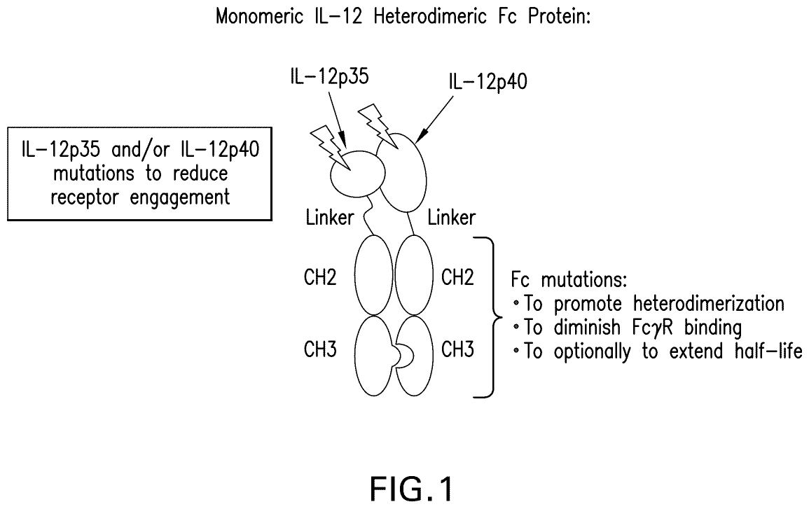

The present invention provides monovalent IL-12 heterodimeric Fc proteins comprising an Fc heterodimer pair wherein a first Fc region of the heterodimer pair is directly or indirectly linked to an IL-12p35 subunit and a second Fc region of the heterodimer pair is directly or indirectly linked to an IL-12p40 subunit and wherein at least one of the IL-12p35 subunit or the IL-12p40 subunit is a modified variant comprising one or more amino acid substitutions or deletions as compared the wild-type IL-12 p35 and p40 subunits, as applicable. In a further embodiment, the resulting monovalent IL-12 heterodimeric Fc protein displays reduced potency compared to a monovalent IL-12 heterodimeric Fc protein comprising wild-type IL-12p35 and IL-12p40 subunits. The Fc region of the monovalent IL-12 heterodimeric Fc proteins of the present invention further includes mutations that promote heterodimerization of the IL-12p35-Fc and IL-12p40-Fc components, reduce effector function (FcγR binding) of the Fc regions, and optionally, extend half-life of the monovalent IL-12 heterodimeric Fc protein. FIG. 1 shows a cartoon representation of the monovalent IL-12 heterodimeric Fc protein of the present invention.

### III.(a): IL-12 Subunits of the Monovalent IL-12 Heterodimeric Fc Proteins

Mature human IL-12p35 and human IL-12p40 subunits comprise the amino acid sequences set forth in SEQ ID NO: 239 and SEQ ID NO: 134, respectively. In particular embodiments of the present invention, the mature human IL-12p35 subunit of the monovalent IL-12 heterodimeric Fc protein further comprises an N-terminal deletion of amino acids RNLPVA (SEQ ID NO: 238), which produces a truncated IL12p35 subunit comprising the amino acid sequence set forth in SEQ ID NO: 133. For IL-12p35, the first 10 amino acids is unresolved in the crystal structure of IL-12 and is an unstructured loop in the model. Deletion of the hexapeptide may improve the stability of the IL-12p35 subunit compared to the IL-12p35 subunit comprising the hexapeptide. Table 3A shows exemplary mutant IL-12p35 subunits comprising the N-terminal hexapeptide (+hexapeptide). Exemplary mutant hexapeptide-truncated IL-12p35 subunits (Δhexapeptide) are shown in Table 3B and exemplary IL-12p40 subunits (mutation variants of human IL-12p40 subunit) are shown in Table 4.

TABLE 3A
IL-12p35 Subunits

| SEQ ID NO: | Amino acid Substitution |
|---|---|
| 261 | p35 (E40K) |
| 262 | p35 (E40R) |
| 263 | p35 (E40W) |
| 264 | p35 (I41A) |
| 265 | p35 (I41V) |
| 266 | p35 (I41S) |
| 267 | p35 (I41T) |
| 268 | p35 (D42A) |
| 269 | p35 (D42L) |
| 270 | p35 (D42W) |
| 271 | p35 (E44A) |
| 272 | p35 (E44K) |
| 273 | p35 (E44R) |
| 274 | p35 (D159K) |
| 275 | p35 (D159R) |
| 276 | p35 (D159Y) |
| 277 | p35 (D159F) |
| 278 | p35 (Y161S) |
| 279 | p35 (Y161T) |
| 280 | p35 (Y161A) |
| 281 | p35 (Y161W) |
| 282 | p35 (K162E) |
| 283 | P35 (K162D) |
| 284 | p35 (D159K-Y161T) |
| 285 | p35 (F33G-D159K-Y161T) |
| 286 | p35 (F33K-D159K-Y161T) |
| 287 | p35 (F33S-D159K-Y161T) |
| 288 | p35 (Y34G-D159K-Y161T) |
| 289 | p35 (Y34S-D159K-Y161T) |
| 290 | p35 (Y34T-D159K-Y161T) |
| 291 | p35 (P35Q-D159K-Y161T) |
| 292 | p35 (P35E-D159K-Y161T) |
| 293 | p35 (F33S-Y34T D159K-Y161T) |
| 294 | p35 (F33S-Y34S D159K-Y161T) |
| 295 | p35 (F33G-Y161T) |
| 296 | p35 (F33K-Y161T) |
| 297 | p35 (F33S-Y161T) |
| 298 | p35 (Y34G-Y161T) |
| 299 | p35 (Y34S-Y161T) |
| 300 | p35 (Y34T-Y161T) |
| 301 | p35 (F33S-Y34T-Y161T) |
| 302 | p35 (F33S-Y34S-Y161T) |
| 303 | p35 (F33S-Y34G-Y161T) |

TABLE 3B
IL-12p35 Subunits

| SEQ ID NO: | Amino acid Substitution |
|---|---|
| 135 | p35 (E40K) |
| 136 | p35 (E40R) |
| 137 | p35 (E40W) |
| 138 | p35 (I41A) |
| 139 | p35 (I41V) |
| 140 | p35 (I41S) |
| 141 | p35 (I41T) |
| 142 | p35 (D42A) |
| 143 | p35 (D42L) |
| 144 | p35 (D42W) |
| 145 | p35 (E44A) |
| 146 | p35 (E44K) |
| 147 | p35 (E44R) |
| 148 | p35 (D159K) |
| 149 | p35 (D159R) |
| 150 | p35 (D159Y) |
| 151 | p35 (D159F) |
| 152 | p35 (Y161S) |
| 153 | p35(Y161T) |
| 154 | p35(Y161A) |
| 155 | p35(Y161W) |
| 156 | p35 (K162E) |
| 157 | P35 (K162D) |
| 170 | p35 (D159K-Y161T) |
| 171 | p35 (F33G-D159K-Y161T) |
| 172 | p35 (F33K-D159K-Y161T) |
| 173 | p35 (F33S-D159K-Y161T) |
| 174 | p35 (Y34G-D159K-Y161T) |
| 175 | p35 (Y34S-D159K-Y161T) |
| 176 | p35 (Y34T-D159K-Y161T) |
| 177 | p35 (P35Q-D159K-Y161T) |
| 178 | p35 (P35E-D159K-Y161T) |
| 179 | p35 (F33S-Y34T D159K-Y161T) |
| 180 | p35 (F33S-Y34S D159K-Y161T) |
| 181 | p35 (F33G-Y161T) |
| 182 | p35 (F33K-Y161T) |
| 183 | p35 (F33S-Y161T) |
| 184 | p35 (Y34G-Y161T) |
| 185 | p35 (Y34S-Y161T) |
| 186 | p35 (Y34T-Y161T) |
| 187 | p35 (F33S-Y34T-Y161T) |
| 188 | p35 (F33S-Y34S-Y161T) |
| 189 | p35 (F33S-Y34G-Y161T) |

TABLE 4
IL-12p40 Subunits

| SEQ ID NO: | Amino acid Substitution |
|---|---|
| 158 | p40 (K85E) |
| 159 | p40 (K85Y) |
| 160 | p40 (D87A) |
| 161 | p40 (D87L) |
| 162 | p40 (I89A) |
| 163 | p40 (I89S) |
| 164 | p40 (I89D) |
| 165 | p40 (I89P) |
| 166 | p40 (K104E) |
| 167 | p40 (K104D) |
| 168 | p40 (K104W) |
| 169 | P40 (104S) |
| 190 | p40 (D87L-K104E) |
| 191 | p40 (D87L-K104D) |
| 192 | p40 (P143A-P215A) |
| 193 | p40 (P143A-P215A-K260A-R261S-V303I) |
| 194 | p40 (P143A-P215A-K260S-R261S-V303I) |
| 195 | p40 (K260A-R261S-V303I) |
| 196 | p40 (K260S-R261S-V303I) |
| 197 | p40 (K260A-R261S) |
| 198 | p40 (K260S-R261S) |

In particular embodiments, the monovalent IL-12 heterodimeric Fc protein comprises an IL-12p35 subunit comprising the amino acid sequence set forth in SEQ ID NO: 135, 136, 137, 138, 139, 140, 141, 142, 143, 144, 145, 146, 147, 148, 149, 150, 151, 152, 153, 154, 155, 156, or 157 and a wild-type IL-12p40 subunit comprising the amino acid sequence set forth in SEQ ID NO: 134. In particular embodiments, the monovalent IL-12 heterodimeric Fc protein comprises an IL-12p35 subunit comprising the amino acid sequence set forth in SEQ ID NO: 140, 147, 148, 149, 152, 153, or 154 and a wild-type IL-12p40 subunit. In particular embodiments, the monovalent IL-12 heterodimeric Fc protein comprises an IL-12p35 subunit comprising the amino acid sequence set forth in SEQ ID NO: 152 or SEQ ID NO: 153 and a wild-type IL-12p40 subunit comprising the amino acid sequence set forth in SEQ ID NO: 134. In particular embodiments, the monovalent IL-12 heterodimeric Fc protein comprises an IL-12p35 subunit comprising the amino acid sequence set forth in SEQ ID NO: 152 and a wild-type IL-12p40 subunit comprising the amino acid sequence set forth in SEQ ID NO: 134.

In particular embodiments, the monovalent IL-12 heterodimeric Fc protein comprises an IL-12p35 subunit comprising the amino acid sequence set forth in SEQ ID NO: 135, 136, 137, 138, 139, 140, 141, 142, 143, 144, 145, 146, 147, 148, 149, 150, 151, 152, 153, 154, 155, 156, or 157 and an IL-12p40 subunit comprising the amino acid sequence set forth in SEQ ID NO: 192, 193, 194, 195, 196, 197, or 198. In particular embodiments, the monovalent IL-12 heterodimeric Fc protein comprises an IL-12p35 subunit comprising the amino acid sequence set forth in SEQ ID NO: 140, 147, 148, 149, 152, 153, or 154 and an IL-12p40 subunit comprising the amino acid sequence set forth in SEQ ID NO: 192, 193, 194, 195, 196, 197, or 198. In particular embodiments, the monovalent IL-12 heterodimeric Fc protein comprises an IL-12p35 subunit comprising the amino acid sequence set forth in SEQ ID NO: 152 or SEQ ID NO: 153 and an IL-12p40 subunit comprising the amino acid sequence set forth in SEQ ID NO: 192, 193, 194, 195, 196, 197, or 198. In particular embodiments, the monovalent IL-12 heterodimeric Fc protein comprises an IL-12p35 subunit comprising the amino acid sequence set forth in SEQ ID NO: 152 and an IL-12p40 subunit comprising the amino acid sequence set forth in SEQ ID NO: 192, 193, 194, 195, 196, 197, or 198.

In particular embodiments, the monovalent IL-12 heterodimeric Fc protein comprises an IL-12p35 subunit comprising the amino acid sequence set forth in SEQ ID NO: 261, 262, 263, 264, 265, 266, 267, 268, 269, 270, 271, 272, 273, 274, 275, 276, 277, 278, 279, 280, 281, or 283 and a wild-type IL-12p40 subunit comprising the amino acid sequence set forth in SEQ ID NO: 134. In particular embodiments, the monovalent IL-12 heterodimeric Fc protein comprises an IL-12p35 subunit comprising the amino acid sequence set forth in SEQ ID NO: 266, 273, 274, 275, 278, or 279 and a wild-type IL-12p40 subunit comprising the amino acid sequence set forth in SEQ ID NO: 134. In particular embodiments, the monovalent IL-12 heterodimeric Fc protein comprises an IL-12p35 subunit comprising the amino acid sequence set forth in SEQ ID NO: 278 or SEQ ID NO: 279 and a wild-type IL-12p40 subunit comprising the amino acid sequence set forth in SEQ ID NO: 134. In particular embodiments, the monovalent IL-12 heterodimeric Fc protein comprises an IL-12p35 subunit comprising the amino acid sequence set forth in SEQ ID NO: 278 and a wild-type IL-12p40 subunit comprising the amino acid sequence set forth in SEQ ID NO: 134.

In particular embodiments, the monovalent IL-12 heterodimeric Fc protein comprises an IL-12p35 subunit comprising the amino acid sequence set forth in SEQ ID NO: 261, 262, 263, 264, 265, 266, 267, 268, 269, 270, 271, 272, 273, 274, 275, 276, 277, 278, 279, 280, 281, or 283 and an IL-12p40 subunit comprising the amino acid sequence set forth in SEQ ID NO: 192, 193, 194, 195, 196, 197, or 198. In particular embodiments, the monovalent IL-12 heterodimeric Fc protein comprises an IL-12p35 subunit comprising the amino acid sequence set forth in SEQ ID NO: 266, 273, 274, 275, 278, or 279 and an IL-12p40 subunit comprising the amino acid sequence set forth in SEQ ID NO: 192, 193, 194, 195, 196, 197, or 198. In particular embodiments, the monovalent IL-12 heterodimeric Fc protein comprises an IL-12p35 subunit comprising the amino acid sequence set forth in SEQ ID NO: 278 or SEQ ID NO: 279 and an IL-12p40 subunit comprising the amino acid sequence set forth in SEQ ID NO: 192, 193, 194, 195, 196, 197, or 198. In particular embodiments, the monovalent IL-12 heterodimeric Fc protein comprises an IL-12p35 subunit comprising the amino acid sequence set forth in SEQ ID NO: 278 and an IL-12p40 subunit comprising the amino acid sequence set forth in SEQ ID NO: 192, 193, 194, 195, 196, 197, or 198.

In particular embodiments, the monovalent IL-12 heterodimeric Fc protein comprises an IL-12p35 subunit comprising the amino acid sequence set forth in SEQ ID NO: 170, 171, 172, 173, 174, 175, 176, 177, 178, 179, 180, 181, 182, 183, 184, 185, 186, 187, 188, or 189 and a wild-type IL-12p40 subunit comprising the amino acid sequence set forth in SEQ ID NO: 134.

In particular embodiments, the monovalent IL-12 heterodimeric Fc protein comprises an IL-12p35 subunit comprising the amino acid sequence set forth in SEQ ID NO: 170, 171, 172, 173, 174, 175, 176, 177, 178, 179, 180, 181, 182, 183, 184, 185, 186, 187, 188, or 189 and an IL-12p40 subunit comprising the amino acid sequence set forth in SEQ ID NO: 192, 193, 194, 195, 196, 197, or 198.

In particular embodiments, the monovalent IL-12 heterodimeric Fc protein comprises an IL-12p35 subunit comprising the amino acid sequence set forth in SEQ ID NO: 284, 285, 286, 287, 288, 289, 290, 291, 292, 293, 294, 295, 296, 297, 298, 299, 300, 301, 302, or 303 and a wild-type IL-12p40 subunit comprising the amino acid sequence set forth in SEQ ID NO: 134.

In particular embodiments, the monovalent IL-12 heterodimeric Fc protein comprises an IL-12p35 subunit comprising the amino acid sequence set forth in SEQ ID NO: 284, 285, 286, 287, 288, 289, 290, 291, 292, 293, 294, 295, 296, 297, 298, 299, 300, 301, 302, or 303 and an IL-12p40 subunit comprising the amino acid sequence set forth in SEQ ID NO: 192, 193, 194, 195, 196, 197, or 198.

In particular embodiments, the monovalent IL-12 heterodimeric Fc protein comprises an IL-12p35 subunit comprising the amino acid sequence set forth in SEQ ID NO: 133 and an IL-12p40 subunit comprising the amino acid sequence set forth in SEQ ID NO: 158, 159, 160, 161, 162, 163, 164, 165, 166, 167, 168, or 169. In particular embodiments, the monovalent IL-12 heterodimeric Fc protein comprises an IL-12p35 subunit comprising the amino acid sequence set forth in SEQ ID NO: 133 and an IL-12p40 subunit comprising the amino acid sequence set forth in SEQ ID NO: 161, 190, or 191.

In particular embodiments, the monovalent IL-12 heterodimeric Fc protein comprises a non-truncated IL-12p35 subunit (amino acid sequence set forth in SEQ ID NO: 239) and an IL-12p40 subunit comprising the amino acid sequence set forth in SEQ ID NO: 134. In particular embodiments, the monovalent IL-12 heterodimeric Fc protein comprises a non-truncated IL-12p35 subunit (amino acid sequence set forth in SEQ ID NO: 239) and an IL-12p40 subunit comprising an amino acid sequence set forth in SEQ ID NO: 158, 159, 160, 161, 162, 163, 164, 165, 166, 167, 168, or 169. In particular embodiments, the monovalent IL-12 heterodimeric Fc protein comprises a non-truncated IL-12p35 subunit (SEQ ID NO: 239) and an IL-12p40 subunit comprising the amino acid sequence set forth in SEQ ID NO: 161, 190, or 191.

In a further embodiments, the IL-12p40 subunit further comprises a P143A-P215A, P143A-P215A-K260A-R261S-V303I, P143A-P215A-K260S-R261S-V303I, K260A-R261S-V303I, K260S-R261S-V303I, K260A-R261S, or K260S-R261S amino acid substitution. These amino acid substitutions may enhance the resistance of the IL-12p40 subunit to undergo protease clipping under certain protein production conditions. Exemplary mutated IL-12p40 subunits comprise an amino acid sequence set forth in SEQ ID NO: 126, SEQ ID NO: 127, SEQ ID NO: 128, SEQ ID NO: 129, SEQ ID NO: 130, SEQ ID NO: 131, or SEQ ID NO: 132.

### III.(b): Monovalent IL-12 Heterodimeric Fc Protein

The monovalent IL-12 heterodimeric Fc protein comprises a first Fc region and a second Fc region heterodimer pair. An IL-12p35 subunit disclosed herein may be directly linked or indirectly linked via a peptide linker or non-peptidyl polymer to the first Fc region or second Fc region of the heterodimer pair to provide an IL-12p35 Fc region protein; and an IL-12p40 subunit disclosed herein may be directly linked or indirectly linked via a peptide linker or non-peptidyl polymer to the Fc region of the heterodimer pair not linked to the IL-12p35 subunit to provide an IL-12p40 Fc region protein; with the proviso that the IL-12p35 and IL12p40 subunits are linked to their respective Fc region in an orientation that permits the two subunits to form a functional IL-12 protein. In some embodiments, the C-terminal amino acid of the IL-12p35 and IL-12p40 subunits are directly or indirectly linked via a peptide linker or non-peptidyl polymer to the N-terminal amino acid of the first and second Fc regions, respectively. In some embodiments, the N-terminal amino acid of the IL-12p35 and IL-12p40 subunits are directly or indirectly linked via a peptide linker or non-peptidyl polymer to the C-terminal amino acid of the first and second Fc regions, respectively.

In particular embodiments of the present invention, the C-terminal amino acid of the IL-12p35 and IL-12p40 subunits are indirectly linked via peptide linkers to the N-terminal amino acid of the first and second Fc regions, respectively. In some embodiments, the N-terminal amino acid of the IL-12p35 and IL-12p40 subunits are indirectly linked via peptide linkers to the C-terminal amino acid of the first and second Fc regions, respectively. Peptide linkers for linking the IL-12 subunits to their respective Fc region can vary from 10 to 25 amino acids in length and are typically, but not always, composed of repeating units of hydrophilic amino acids such as glycine (G) and serine (S) having the sequence G4S (SEQ ID NO: 204), for example, (G4S)ₙ, wherein n is 1, 2, 3, 4, or 5 (SEQ ID NO: 205) or (G4S)ₙ, wherein is 1 to 10 peptide units (SEQ ID NO: 207). Peptide linkers of shorter lengths (2-4 amino acids) have also been used such as 4G (SEQ ID NO: 202). Generally, the (G4S)₂ peptide (SEQ ID NO: 203) comprising two repeating G4S units (SEQ ID NO: 204) or the (G4S)₃ peptide (SEQ ID NO: 206) comprising three repeating G4S units (SEQ ID NO: 204) are commonly used for generating Fc region fusion proteins. The peptide linkers for linking each subunit are chosen so that in the monovalent IL-12 heterodimeric Fc protein, the IL-12p35 and IL-12p40 subunits are positioned with respect to each other such as to allow them to associate in an orientation that permits formation of a functional IL-12 protein.

In particular embodiments of the present invention, the C-terminal amino acid of the IL-12p35 and IL-12p40 subunits are indirectly linked via peptide linkers to the N-terminal amino acid of the first and second Fc regions, respectively, wherein the IL-12p35 subunit is linked by a (G4S)ₙ peptide, wherein n is 1, 2, 3, 4, or 5 (SEQ ID NO: 205) and the IL-12p40 subunit is linked by a 4G peptide (SEQ ID NO: 202).

In some embodiments, the N-terminal amino acid of the IL-12p35 and IL-12p40 subunits are indirectly linked via peptide linkers to the C-terminal amino acid of the first and second Fc regions, respectively wherein the IL-12p35 subunit is linked by a (G4S)ₙ peptide, wherein n is 1, 2, 3, 4, or 5 (SEQ ID NO: 205) and the IL-12p40 subunit is linked by a 4G peptide (SEQ ID NO: 202).

In particular embodiments of the present invention, the C-terminal amino acid of the IL-12p35 and IL-12p40 subunits are indirectly linked via peptide linkers to the N-terminal amino acid of the first and second Fc regions, respectively, wherein the IL-12p35 subunit is linked by a (G4S)₃ peptide (SEQ ID NO: 206) and the IL-12p40 subunit is linked by a 4G peptide (SEQ ID NO: 202). In some embodiments, the N-terminal amino acid of the IL-12p35 and IL-12p40 subunits are indirectly linked via peptide linkers to the C-terminal amino acid of the first and second Fc regions, respectively wherein the IL-12p35 subunit is linked by a (G4S)₃ peptide (SEQ ID NO: 206) and the IL-12p40 subunit is linked by a 4G peptide (SEQ ID NO: 202).

The non-peptidyl polymer may be a biocompatible polymer including two or more repeating units linked to each other by any covalent bond excluding a peptide bond. The non-peptidyl polymers useful in the present invention may be selected from the group consisting of a biodegradable polymer, a lipid polymer, chitin, hyaluronic acid, and a combination thereof. In particular embodiments, the biodegradable polymer may be polyethylene glycol, polypropylene glycol, ethylene glycol-propylene glycol copolymer, polyoxyethylated polyol, polyvinyl alcohol, polysaccharide, dextran, polyvinyl ethyl ether, polylactic acid (PLA) or polylactic-glycolic acid (PLGA), and more preferably, is polyethylene glycol (PEG). In addition, derivatives thereof known in the art and derivatives easily prepared by a method known in the art are included in the scope of the present invention. The non-peptidyl polymers for linking each subunit are chosen so that in the monovalent IL-12 heterodimeric Fc protein, the IL-12p35 and IL-12p40 subunits are positioned with respect to each other to allow them to associate in an orientation that permits formation of a functional IL-12 protein. In particular embodiments, the non-peptidyl polymer linking the IL-12p35 subunit to the Fc region is about 22 Å in length and the non-peptidyl polymer linking the IL-12p40 subunit to the Fc region is about 6 Å in length. In particular embodiments, the non-peptidyl polymer linking the IL-12p35 subunit to the Fc region is about 8 PEG units (352 Daltons) and the non-peptidyl polymer linking the IL-12p40 subunit to the Fc region is about 2 PEG units (88 Daltons). In general, a desirable ratio to be maintained is about 1:4 in non-peptidyl polymer lengths.

### III.(c): Heterodimerization

The monovalent IL-12 heterodimeric Fc protein of the present invention comprises a first Fc region and a second Fc region, each comprising the Fc region from an IgA, IgG, IgD, IgE, or IgM comprising one or more amino acid substitutions that promote formation of an Fc region heterodimeric pair. The Fc region comprises a CH3 domain and the hinge region or part of the hinge region. Amino acid modifications that promote formation of heterodimers have been disclosed in WO9850431, which discloses a knob-in-hole (KIH) method for creating Fc region heterodimers. In the KIH method, one Fc region of the heterodimer pair comprises amino acid substitutions that create a protuberance that extends outward from surface of the Fc region (knob) that fits into a hole created by appropriate amino acid substitutions in the other Fc region of the heterodimeric pair, which promotes heterodimer formation over homodimer formation. An example of amino acid substitutions include S354C:T366W amino acid substitutions to a first Fc region to form the knob and Y349C:T366S:L368A:Y407V amino acid substitutions in a second Fc region to form the hole (amino acid numbering according to EU Index) wherein the first and second fc regions form a heterodimer pair. WO2014084607 discloses KIH in which the Fc heterodimer comprises a first Fc region comprising a K409W amino acid substitution to form the knob and a second Fc region comprising D399V and F405T amino acid substitutions to form the hole (amino acid numbering according to EU Index) wherein the first and second fc regions form a heterodimer pair. WO2013063702 discloses KIH in which the Fc heterodimer comprises a first Fc region comprising amino acid modifications at positions T350, L351, F405, and Y407, and the second Fc region comprises amino acid modifications at positions T350, T366, K392 and T394 (amino acid numbering according to EU Index) wherein the first and second fc regions form a heterodimer pair.

Other methods for promoting Fc region heterodimeric pairs include electrostatic steering as disclosed in WO2009089004 or protein isoelectric point modifications as disclosed in WO2013055809.

In particular embodiments of the monovalent IL-12 heterodimeric Fc protein of the present invention, the Fc region is of IgG1 origin and comprises a first Fc region comprising S354C:T366W amino acid substitutions to form the knob and a second Fc region comprising Y349C:T366S:L368A:Y407V amino acid substitutions to form the hole wherein the first and second fc regions form a heterodimer pair and wherein the amino acid numbering is according to EU Index.

In particular embodiments of the monovalent IL-12 heterodimeric Fc protein of the present invention, the Fc region is of IgG1 origin and comprises a first Fc region comprising 409W amino acid substitution to form the knob and a second Fc region comprising D399V:F405T amino acid substitutions to form the hole wherein the first and second fc regions form a heterodimer pair and wherein the amino acid numbering is according to EU Index.

In particular embodiments of the monovalent IL-12 heterodimeric Fc protein of the present invention, the Fc region is of IgG1 origin and comprises a first Fc region comprising 357W amino acid substitutions to form the knob and a second Fc region comprising Y349S amino acid substitution to form the hole wherein the first and second fc regions form a heterodimer pair and wherein the amino acid numbering is according to EU Index.

### III.(d): Effector Function Modifications of the Fc Region

In further embodiments of the monovalent IL-12 heterodimeric Fc protein, the first and second Fc regions further comprise one or more amino acid substitutions that reduce or ablate effector function of the Fc region. In particular embodiments, the first and second Fc regions further comprise:
(i) E233A and L235A (EALA) amino acid substitutions, wherein the numbering is according to Eu Index;
(ii) L234A L235A D265S (LALADS) amino acid substitutions, wherein the numbering is according to Eu Index;
(iii) L234A L235A P329G (LALAPG) amino acid substitutions, wherein the numbering is according to Eu Index;
(iv) L235E (LE) amino acid substitutions, wherein the numbering is according to Eu Index;
(v) D265A (DA) amino acid substitution, wherein the numbering is according to Eu Index;
(vi) D265A N297G (DANG) amino acid substitutions, wherein the numbering is according to Eu Index;
(vii) N297X amino acid substitution, wherein X is any amino acid other than N, wherein the numbering is according to Eu Index; or
(viii) N297A/D356E/L358M (NADELM) amino acid substitutions, wherein the numbering is according to Eu Index.

In particular embodiments of the monovalent IL-12 heterodimeric Fc protein, the IL-12p35 Fc region protein comprises an IL-12p35 subunit having an amino acid sequence set forth in SEQ ID NO: 1-25 or 38-57, and the IL-12p40 Fc region protein comprises an IL-12p40 subunit having an amino acid sequence set forth in SEQ ID NO: 26-37 or 58-66, wherein the Fc regions further comprise one or more amino acid substitutions that reduces or ablates effector function and one or more amino acid substitutions that promote heterodimerization of the IL-12p35 Fc region protein and IL-12p40 Fc region protein.

In particular embodiments of the monovalent IL-12 heterodimeric Fc protein, the first Fc region comprises SEQ ID NO: 208, 212, or 216 and the second Fc region comprises an amino acid sequence set forth in SEQ ID NO: 209, 213, or 217.

Exemplary IL-12 heterodimeric Fc proteins of the present invention in which the IL-12p35 Fc region protein comprises an Fc (LALADS-KIH-Knob) and the IL-12p40 Fc region protein comprises an Fc (LALADS-KIH-Hole) are shown in Tables 5-15.

TABLE 5

| | IL-12p35-Fc | | IL-12p40-Fc | |
|---|---|---|---|---|
| | (LALADS-KIH-Knob) | | (LALADS-KIH-Hole) | |
| | IL-12p35 | SEQ | IL-12p40 | SEQ |
| IL-12 Analog | subunit | ID NO: | subunit | ID NO: |
| IL-12-Fc-1 | p35 E40K | 3 | WT | 2 |
| IL-12-Fc-2 | p35 E40R | 4 | WT | 2 |
| IL-12-Fc-3 | p35 E40W | 5 | WT | 2 |
| IL-12-Fc-4 | p35 I41A | 6 | WT | 2 |
| IL-12-Fc-5 | p35 I41V | 7 | WT | 2 |
| IL-12-Fc-6 | p35 I41S | 8 | WT | 2 |
| IL-12-Fc-7 | p35 I41T | 9 | WT | 2 |
| IL-12-Fc-8 | p35 D42A | 10 | WT | 2 |
| IL-12-Fc-9 | p35 D42L | 11 | WT | 2 |
| IL-12-Fc-10 | p35 D42W | 12 | WT | 2 |
| IL-12-Fc-11 | p35 E44A | 13 | WT | 2 |
| IL-12-Fc-12 | p35 E44K | 14 | WT | 2 |
| IL-12-Fc-13 | p35 E44R | 15 | WT | 2 |
| IL-12-Fc-14 | p35 D159K | 16 | WT | 2 |
| IL-12-Fc-15 | p35 D159R | 17 | WT | 2 |
| IL-12-Fc-16 | p35 D159Y | 18 | WT | 2 |
| IL-12-Fc-17 | p35 D159F | 19 | WT | 2 |
| IL-12-Fc-18 | p35 Y161S | 20 | WT | 2 |
| IL-12-Fc-19 | p35 Y161T | 21 | WT | 2 |
| IL-12-Fc-20 | p35 Y161A | 22 | WT | 2 |
| IL-12-Fc-21 | p35 Y161W | 23 | WT | 2 |
| IL-12-Fc-22 | p35 K162E | 24 | WT | 2 |
| IL-12-Fc-23 | p35 K162D | 25 | WT | 2 |

WT = wild-type amino acid sequence
IL-12p35 subunits have N-terminal hexapeptide truncation

TABLE 6

| | p35-Fc | | p40-Fc | |
|---|---|---|---|---|
| | (LALADS-KIH-Knob) | | (LALADS-KIH-Hole) | |
| | IL-12p35 | SEQ | IL-12p40 | SEQ |
| IL-12-Analog | Subunit | ID NO: | Subunit | ID NO: |
| IL-12-Fc-24 | WT | 1 | p40 K85E | 26 |
| IL-12-Fc-25 | WT | 1 | p40 K85Y | 27 |
| IL-12-Fc-26 | WT | 1 | p40 D87A | 28 |
| IL-12-Fc-27 | WT | 1 | p40 D87L | 29 |
| IL-12-Fc-28 | WT | 1 | p40 I89A | 30 |
| IL-12-Fc-29 | WT | 1 | p40 I89S | 31 |
| IL-12-Fc-30 | WT | 1 | p40 I89D | 32 |
| IL-12-Fc-31 | WT | 1 | p40 I89P | 33 |
| IL-12-Fc-32 | WT | 1 | p40 K104E | 34 |
| IL-12-Fc-33 | WT | 1 | p40 K104D | 35 |
| IL-12-Fc-34 | WT | 1 | p40 K104W | 36 |
| IL-12-Fc-35 | WT | 1 | p40 K104S | 37 |

WT = wild-type amino acid sequence
IL-12p35 subunits have N-terminal hexapeptide truncation

TABLE 7

| | p35-Fc | | p40-Fc | |
|---|---|---|---|---|
| | (LALADS-KIH-Knob) | | (LALADS-KIH-Hole) | |
| | IL-12p35 | SEQ | IL-12p40 | SEQ |
| IL-12-Analog | Subunit | ID NO: | Subunit | ID NO: |
| IL-12-Fc-36 | p35 I41S | 8 | p40 D87L | 29 |
| IL-12-Fc-37 | p35 E44R | 15 | p40 D87L | 29 |
| IL-12-Fc-38 | p35 D159K | 16 | p40 D87L | 29 |
| IL-12-Fc-39 | p35 D159R | 17 | p40 D87L | 29 |
| IL-12-Fc-40 | p35 Y161S | 20 | p40 D87L | 29 |
| IL-12-Fc-41 | p35 Y161T | 21 | p40 D87L | 29 |
| IL-12-Fc-42 | p35 Y161A | 22 | p40 D87L | 29 |
| IL-12-Fc-43 | p35 I41S | 8 | p40 K104E | 34 |
| IL-12-Fc-44 | p35 E44R | 15 | p40 K104E | 34 |
| IL-12-Fc-45 | p35 D159K | 16 | p40 K104E | 34 |
| IL-12-Fc-46 | p35 D159R | 17 | p40 K104E | 34 |
| IL-12-Fc-47 | p35 Y161S | 20 | p40 K104E | 34 |
| IL-12-Fc-48 | p35 Y161T | 21 | p40 K104E | 34 |
| IL-12-Fc-49 | p35 Y161A | 22 | p40 K104E | 34 |
| IL-12-Fc-50 | p35 I41S | 8 | p40 K104D | 35 |
| IL-12-Fc-51 | p35 E44R | 15 | p40 K104D | 35 |
| IL-12-Fc-52 | p35 D159K | 16 | p40 K104D | 35 |
| IL-12-Fc-53 | p35 D159R | 17 | p40 K104D | 35 |
| IL-12-Fc-54 | p35 Y161S | 20 | p40 K104D | 35 |
| IL-12-Fc-55 | p35 Y161T | 21 | p40 K104D | 35 |
| IL-12-Fc-56 | p35 Y161A | 22 | p40 K104D | 35 |

IL-12p35 subunits have N-terminal hexapeptide truncation

TABLE 8

| | p35-Fc (LALADS-KIH-Knob) | | p40-Fc (LALADS-KIH Hole) | |
|---|---|---|---|---|
| IL-12-Analog | IL-12p35 Subunit | SEQ ID NO: | IL-12p40 Subunit | SEQ ID NO: |
| IL-12-Fc-57 | p35 Y161T | 21 | p40 D87L K104E | 34 |
| IL-12-Fc-58 | p35 Y161A | 22 | p40 D87L K104D | 35 |
| IL-12-Fc-59 | p35 D159K Y161T | 38 | p40 D87L K104E | 34 |
| IL-12-Fc-60 | p35 D159K Y161T | 38 | p40 D87L K104D | 35 |
| IL-12-Fc-61 | p35 F33G D159K Y161T | 39 | p40 D87L K104E | 34 |
| IL-12-Fc-62 | p35 F33K D159K Y161T | 40 | p40 D87L K104E | 34 |
| IL-12-Fc-63 | p35 F33S D159K Y161T | 41 | p40 D87L K104E | 34 |
| IL-12-Fc-64 | p35 Y34G D159K Y161T | 42 | p40 D87L K104E | 34 |
| IL-12-Fc-65 | p35 Y34S D159K Y161T | 43 | p40 D87L K104E | 34 |
| IL-12-Fc-66 | p35 Y34T D159K Y161T | 44 | p40 D87L K104E | 34 |
| IL-12-Fc-67 | p35 P35Q D159K Y161T | 45 | p40 D87L K104E | 34 |
| IL-12-Fc-68 | p35 P35E D159K Y161T | 46 | p40 D87L K104E | 34 |
| IL-12-Fc-69 | p35 F33S Y34T D159K Y161T | 47 | p40 D87L K104E | 34 |
| IL-12-Fc-70 | p35 F33S Y34S D159K Y161T | 48 | p40 D87L K104E | 34 |
| IL-12-Fc-71 | p35 F33G Y161T | 49 | p40 D87L K104E | 34 |
| IL-12-Fc-72 | p35 F33K Y161T | 50 | p40 D87L K104E | 34 |
| IL-12-Fc-73 | p35 F33S Y161T | 51 | p40 D87L K104E | 34 |
| IL-12-Fc-74 | p35 Y34G Y161T | 52 | p40 D87L K104E | 34 |
| IL-12-Fc-75 | p35 Y34S Y161T | 53 | p40 D87L K104E | 34 |
| IL-12-Fc-76 | p35 Y34T Y161T | 54 | p40 D87L K104E | 34 |
| IL-12-Fc-77 | p35 F33S Y34T Y161T | 55 | p40 D87L K104E | 34 |
| IL-12-Fc-78 | p35 F33S Y34S Y161T | 56 | p40 D87L K104E | 34 |
| IL-12-Fc-79 | p35 F33S Y34G Y161T | 57 | p40 D87L K104E | 34 |

IL-12p35 subunits have N-terminal hexapeptide truncation

TABLE 9

| | p35-Fc | | p40-Fc | |
|---|---|---|---|---|
| | (LALADS-KIH-Knob) | | (LALADS-KIH-Hole) | |
| | IL-12p35 | SEQ | IL-12p40 | SEQ |
| IL-12-Analog | Subunit | ID NO: | Subunit | ID NO: |
| IL-12-Fc-80 | p35 E40K | 3 | p40 143A P215A | 60 |
| IL-12-Fc-81 | p35 E40R | 4 | p40 143A P215A | 60 |
| IL-12-Fc-82 | p35 E40W | 5 | p40 143A P215A | 60 |
| IL-12-Fc-83 | p35 I41A | 6 | p40 143A P215A | 60 |
| IL-12-Fc-84 | p35 I41V | 7 | p40 143A P215A | 60 |
| IL-12-Fc-85 | p35 I41S | 8 | p40 143A P215A | 60 |
| IL-12-Fc-86 | p35 I41T | 9 | p40 143A P215A | 60 |
| IL-12-Fc-87 | p35 D42A | 10 | p40 143A P215A | 60 |
| IL-12-Fc-88 | p35 D42L | 11 | p40 143A P215A | 60 |
| IL-12-Fc-89 | p35 D42W | 12 | p40 143A P215A | 60 |
| IL-12-Fc-90 | p35 E44A | 13 | p40 143A P215A | 60 |
| IL-12-Fc-91 | p35 E44K | 14 | p40 143A P215A | 60 |
| IL-12-Fc-92 | p35 E44R | 15 | p40 143A P215A | 60 |
| IL-12-Fc-93 | p35 D159K | 16 | p40 143A P215A | 60 |
| IL-12-Fc-94 | p35 D159R | 17 | p40 143A P215A | 60 |
| IL-12-Fc-95 | p35 D159Y | 18 | p40 143A P215A | 60 |
| IL-12-Fc-96 | p35 D159F | 19 | p40 143A P215A | 60 |
| IL-12-Fc-97 | p35 Y161S | 20 | p40 143A P215A | 60 |
| IL-12-Fc-98 | p35 Y161T | 21 | p40 143A P215A | 60 |
| IL-12-Fc-99 | p35 Y161A | 22 | p40 143A P215A | 60 |
| IL-12-Fc-100 | p35 Y161W | 23 | p40 143A P215A | 60 |
| IL-12-Fc-101 | p35 K162E | 24 | p40 143A P215A | 60 |
| IL-12-Fc-102 | p35 K162D | 25 | p40 143A P215A | 60 |

IL-12p35 subunits have N-terminal hexapeptide truncation

TABLE 10

| | p35-Fc (LALADS-KIH-Knob) | | p40-Fc (LALADS-KIH-Hole) | |
|---|---|---|---|---|
| IL-12-Analog | IL-12p35 Subunit | SEQ ID NO: | IL-12p40 Subunit | SEQ ID NO: |
| IL-12-Fc-103 | p35 E40K | 3 | p40 P143A P215A K260A R261S V303I | 61 |
| IL-12-Fc-104 | p35 E40R | 4 | p40 P143A P215A K260A R261S V303I | 61 |
| IL-12-Fc-105 | p35 E40W | 5 | p40 P143A P215A K260A R261S V303I | 61 |
| IL-12-Fc-106 | p35 I41A | 6 | p40 P143A P215A K260A R261S V303I | 61 |
| IL-12-Fc-107 | p35 I41V | 7 | p40 P143A P215A K260A R261S V303I | 61 |
| IL-12-Fc-108 | p35 I41S | 8 | p40 P143A P215A K260A R261S V303I | 61 |
| IL-12-Fc-109 | p35 I41T | 9 | p40 P143A P215A K260A R261S V303I | 61 |
| IL-12-Fc-110 | p35 D42A | 10 | p40 P143A P215A K260A R261S V303I | 61 |
| IL-12-Fc-111 | p35 D42L | 11 | p40 P143A P215A K260A R261S V303I | 61 |
| IL-12-Fc-112 | p35 D42W | 12 | p40 P143A P215A K260A R261S V303I | 61 |
| IL-12-Fc-113 | p35 E44A | 13 | p40 P143A P215A K260A R261S V303I | 61 |
| IL-12-Fc-114 | p35 E44K | 14 | p40 P143A P215A K260A R261S V303I | 61 |
| IL-12-Fc-115 | p35 E44R | 15 | p40 P143A P215A K260A R261S V303I | 61 |
| IL-12-Fc-116 | p35 D159K | 16 | p40 P143A P215A K260A R261S V303I | 61 |
| IL-12-Fc-117 | p35 D159R | 17 | p40 P143A P215A K260A R261S V303I | 61 |
| IL-12-Fc-118 | p35 D159Y | 18 | p40 P143A P215A K260A R261S V303I | 61 |
| IL-12-Fc-119 | p35 D159F | 19 | p40 P143A P215A K260A R261S V303I | 61 |
| IL-12-Fc-120 | p35 Y161S | 20 | p40 P143A P215A K260A R261S V303I | 61 |
| IL-12-Fc-121 | p35 Y161T | 21 | p40 P143A P215A K260A R261S V303I | 61 |
| IL-12-Fc-122 | p35 Y161A | 22 | p40 P143A P215A K260A R261S V303I | 61 |
| IL-12-Fc-123 | p35 Y161W | 23 | p40 P143A P215A K260A R261S V303I | 61 |
| IL-12-Fc-124 | p35 K162E | 24 | p40 P143A P215A K260A R261S V303I | 61 |
| IL-12-Fc-125 | p35 K162D | 25 | p40 P143A P215A K260A R261S V303I | 61 |

IL-12p35 subunits have N-terminal hexapeptide truncation

TABLE 11

| | p35-Fc (LALADS-KIH-Knob) | | p40-Fc (LALADS-KIH-Hole) | |
|---|---|---|---|---|
| IL-12-Analog | IL-12p35 Subunit | SEQ ID NO: | IL-12p40 Subunit | SEQ ID NO: |
| IL-12-Fc-126 | p35 E40K | 3 | p40 P143A P215A K260S R261S V303I | 62 |
| IL-12-Fc-127 | p35 E40R | 4 | p40 P143A P215A K260S R261S V303I | 62 |
| IL-12-Fc-128 | p35 E40W | 5 | p40 P143A P215A K260S R261S V303I | 62 |
| IL-12-Fc-129 | p35 I41A | 6 | p40 P143A P215A K260S R261S V303I | 62 |
| IL-12-Fc-130 | p35 I41V | 7 | p40 P143A P215A K260S R261S V303I | 62 |
| IL-12-Fc-131 | p35 I41S | 8 | p40 P143A P215A K260S R261S V303I | 62 |
| IL-12-Fc-132 | p35 I41T | 9 | p40 P143A P215A K260S R261S V303I | 62 |
| IL-12-Fc-133 | p35 D42A | 10 | p40 P143A P215A K260S R261S V303I | 62 |
| IL-12-Fc-134 | p35 D42L | 11 | p40 P143A P215A K260S R261S V303I | 62 |
| IL-12-Fc-135 | p35 D42W | 12 | p40 P143A P215A K260S R261S V303I | 62 |
| IL-12-Fc-136 | p35 E44A | 13 | p40 P143A P215A K260S R261S V303I | 62 |
| IL-12-Fc-137 | p35 E44K | 14 | p40 P143A P215A K260S R261S V303I | 62 |
| IL-12-Fc-138 | p35 E44R | 15 | p40 P143A P215A K260S R261S V303I | 62 |
| IL-12-Fc-139 | p35 D159K | 16 | p40 P143A P215A K260S R261S V303I | 62 |
| IL-12-Fc-140 | p35 D159R | 17 | p40 P143A P215A K260S R261S V303I | 62 |
| IL-12-Fc-141 | p35 D159Y | 18 | p40 P143A P215A K260S R261S V303I | 62 |
| IL-12-Fc-142 | p35 D159F | 19 | p40 P143A P215A K260S R261S V303I | 62 |
| IL-12-Fc-143 | p35 Y161S | 20 | p40 P143A P215A K260S R261S V303I | 62 |
| IL-12-Fc-144 | p35 Y161T | 21 | p40 P143A P215A K260S R261S V303I | 62 |
| IL-12-Fc-145 | p35 Y161A | 22 | p40 P143A P215A K260S R261S V303I | 62 |
| IL-12-Fc-146 | p35 Y161W | 23 | p40 P143A P215A K260S R261S V303I | 62 |
| IL-12-Fc-147 | p35 K162E | 24 | p40 P143A P215A K260S R261S V303I | 62 |
| IL-12-Fc-148 | p35 K162D | 25 | p40 P143A P215A K260S R261S V303I | 62 |

IL-12p35 subunits have N-terminal hexapeptide truncation

TABLE 12

| | p35-Fc (LALADS-KIH-Knob) | | p40-Fc (LALADS-KIH-Hole) | |
|---|---|---|---|---|
| IL-12-Analog | IL-12p35 Subunit | SEQ ID NO: | IL-12p40 Subunit | SEQ ID NO: |
| IL-12-Fc-149 | p35 E40K | 3 | p40 K260A R261S V303I | 63 |
| IL-12-Fc-150 | p35 E40R | 4 | p40 K260A R261S V303I | 63 |
| IL-12-Fc-151 | p35 E40W | 5 | p40 K260A R261S V303I | 63 |
| IL-12-Fc-152 | p35 I41A | 6 | p40 K260A R261S V303I | 63 |
| IL-12-Fc-153 | p35 I41V | 7 | p40 K260A R261S V303I | 63 |
| IL-12-Fc-154 | p35 I41S | 8 | p40 K260A R261S V303I | 63 |
| IL-12-Fc-155 | p35 I41T | 9 | p40 K260A R261S V303I | 63 |
| IL-12-Fc-156 | p35 D42A | 10 | p40 K260A R261S V303I | 63 |
| IL-12-Fc-157 | p35 D42L | 11 | p40 K260A R261S V303I | 63 |
| IL-12-Fc-158 | p35 D42W | 12 | p40 K260A R261S V303I | 63 |
| IL-12-Fc-159 | p35 E44A | 13 | p40 K260A R261S V303I | 63 |
| IL-12-Fc-160 | p35 E44K | 14 | p40 K260A R261S V303I | 63 |
| IL-12-Fc-161 | p35 E44R | 15 | p40 K260A R261S V303I | 63 |
| IL-12-Fc-162 | p35 D159K | 16 | p40 K260A R261S V303I | 63 |
| IL-12-Fc-163 | p35 D159R | 17 | p40 K260A R261S V303I | 63 |
| IL-12-Fc-164 | p35 D159Y | 18 | p40 K260A R261S V303I | 63 |
| IL-12-Fc-165 | p35 D159F | 19 | p40 K260A R261S V303I | 63 |
| IL-12-Fc-166 | p35 Y161S | 20 | p40 K260A R261S V303I | 63 |
| IL-12-Fc-168 | p35 Y161T | 21 | p40 K260A R261S V303I | 63 |
| IL-12-Fc-169 | p35 Y161A | 22 | p40 K260A R261S V303I | 63 |
| IL-12-Fc-170 | p35 Y161W | 23 | p40 K260A R261S V303I | 63 |
| IL-12-Fc-171 | p35 K162E | 24 | p40 K260A R261S V303I | 63 |
| IL-12-Fc-172 | p35 K162D | 25 | p40 K260A R261S V303I | 63 |

IL-12p35 subunits have N-terminal hexapeptide truncation

TABLE 13

| | p35-Fc (LALADS-KIH-Knob) | | p40-Fc (LALADS-KIH-Hole) | |
|---|---|---|---|---|
| IL-12-Analog | IL-12p35 Subunit | SEQ ID NO: | IL-12p40 Subunit | SEQ ID NO: |
| IL-12-Fc-173 | p35 E40K | 3 | p40 K260S R261S V303I | 64 |
| IL-12-Fc-174 | p35 E40R | 4 | p40 K260S R261S V303I | 64 |
| IL-12-Fc-175 | p35 E40W | 5 | p40 K260S R261S V303I | 64 |
| IL-12-Fc-176 | p35 I41A | 6 | p40 K260S R261S V303I | 64 |
| IL-12-Fc-177 | p35 I41V | 7 | p40 K260S R261S V303I | 64 |
| IL-12-Fc-178 | p35 I41S | 8 | p40 K260S R261S V303I | 64 |
| IL-12-Fc-179 | p35 I41T | 9 | p40 K260S R261S V303I | 64 |
| IL-12-Fc-180 | p35 D42A | 10 | p40 K260S R261S V303I | 64 |
| IL-12-Fc-181 | p35 D42L | 11 | p40 K260S R261S V303I | 64 |
| IL-12-Fc-182 | p35 D42W | 12 | p40 K260S R261S V303I | 64 |
| IL-12-Fc-183 | p35 E44A | 13 | p40 K260S R261S V303I | 64 |
| IL-12-Fc-184 | p35 E44K | 14 | p40 K260S R261S V303I | 64 |
| IL-12-Fc-185 | p35 E44R | 15 | p40 K260S R261S V303I | 64 |
| IL-12-Fc-186 | p35 D159K | 16 | p40 K260S R261S V303I | 64 |
| IL-12-Fc-187 | p35 D159R | 17 | p40 K260S R261S V303I | 64 |
| IL-12-Fc-188 | p35 D159Y | 18 | p40 K260S R261S V303I | 64 |
| IL-12-Fc-189 | p35 D159F | 19 | p40 K260S R261S V303I | 64 |
| IL-12-Fc-190 | p35 Y161S | 20 | p40 K260S R261S V303I | 64 |
| IL-12-Fc-191 | p35 Y161T | 21 | p40 K260S R261S V303I | 64 |
| IL-12-Fc-192 | p35 Y161A | 22 | p40 K260S R261S V303I | 64 |
| IL-12-Fc-193 | p35 Y161W | 23 | p40 K260S R261S V303I | 64 |
| IL-12-Fc-194 | p35 K162E | 24 | p40 K260S R261S V303I | 64 |
| IL-12-Fc-195 | p35 K162D | 25 | p40 K260S R261S V303I | 64 |

IL-12p35 subunits have N-terminal hexapeptide truncation

TABLE 14

| | p35-Fc (LALADS-KIH-Knob) | | p40-Fc (LALADS-KIH-Hole) | |
|---|---|---|---|---|
| IL-12-Analog | IL-12p35 Subunit | SEQ ID NO: | IL-12p40 Subunit | SEQ ID NO: |
| IL-12-Fc-196 | p35 E40K | 3 | p40 K260A R261S | 65 |
| IL-12-Fc-197 | p35 E40R | 4 | p40 K260A R261S | 65 |
| IL-12-Fc-198 | p35 E40W | 5 | p40 K260A R261S | 65 |
| IL-12-Fc-199 | p35 141A | 6 | p40 K260A R261S | 65 |
| IL-12-Fc-200 | p35 141V | 7 | p40 K260A R261S | 65 |
| IL-12-Fc-201 | p35 I41S | 8 | p40 K260A R261S | 65 |
| IL-12-Fc-202 | p35 141T | 9 | p40 K260A R261S | 65 |
| IL-12-Fc-203 | p35 D42A | 10 | p40 K260A R261S | 65 |
| IL-12-Fc-204 | p35 D42L | 11 | p40 K260A R261S | 65 |
| IL-12-Fc-205 | p35 D42W | 12 | p40 K260A R261S | 65 |
| IL-12-Fc-206 | p35 E44A | 13 | p40 K260A R261S | 65 |
| IL-12-Fc-207 | p35 E44K | 14 | p40 K260A R261S | 65 |
| IL-12-Fc-208 | p35 E44R | 15 | p40 K260A R261S | 65 |
| IL-12-Fc-209 | p35 D159K | 16 | p40 K260A R261S | 65 |
| IL-12-Fc-210 | p35 D159R | 17 | p40 K260A R261S | 65 |
| IL-12-Fc-211 | p35 D159Y | 18 | p40 K260A R261S | 65 |
| IL-12-Fc-212 | p35 D159F | 19 | p40 K260A R261S | 65 |
| IL-12-Fc-213 | p35 Y161S | 20 | p40 K260A R261S | 65 |
| IL-12-Fc-214 | p35 Y161T | 21 | p40 K260A R261S | 65 |
| IL-12-Fc-215 | p35 Y161A | 22 | p40 K260A R261S | 65 |
| IL-12-Fc-216 | p35 Y161W | 23 | p40 K260A R261S | 65 |
| IL-12-Fc-217 | p35 K162E | 24 | p40 K260A R261S | 65 |
| IL-12-Fc-218 | p35 K162D | 25 | p40 K260A R261S | 65 |

IL-12p35 subunits have N-terminal hexapeptide truncation

TABLE 15

| | p35-Fc (LALADS-KIH-Knob) | | p40-Fc (LALADS-KIH-Hole) | |
|---|---|---|---|---|
| IL-12-Analog | IL-12p35 Subunit | SEQ ID NO: | IL-12p40 Subunit | SEQ ID NO: |
| IL-12-Fc-219 | p35 E40K | 3 | p40 K260S R261S | 66 |
| IL-12-Fc-220 | p35 E40R | 4 | p40 K260S R261S | 66 |
| IL-12-Fc-221 | p35 E40W | 5 | p40 K260S R261S | 66 |
| IL-12-Fc-222 | p35 141A | 6 | p40 K260S R261S | 66 |
| IL-12-Fc-223 | p35 141V | 7 | p40 K260S R261S | 66 |
| IL-12-Fc-224 | p35 141S | 8 | p40 K260S R261S | 66 |
| IL-12-Fc-225 | p35 141T | 9 | p40 K260S R261S | 66 |
| IL-12-Fc-226 | p35 D42A | 10 | p40 K260S R261S | 66 |
| IL-12-Fc-227 | p35 D42L | 11 | p40 K260S R261S | 66 |
| IL-12-Fc-228 | p35 D42W | 12 | p40 K260S R261S | 66 |
| IL-12-Fc-229 | p35 E44A | 13 | p40 K260S R261S | 66 |
| IL-12-Fc-230 | p35 E44K | 14 | p40 K260S R261S | 66 |
| IL-12-Fc-231 | p35 E44R | 15 | p40 K260S R261S | 66 |
| IL-12-Fc-232 | p35 D159K | 16 | p40 K260S R261S | 66 |
| IL-12-Fc-233 | p35 D159R | 17 | p40 K260S R261S | 66 |
| IL-12-Fc-234 | p35 D159Y | 18 | p40 K260S R261S | 66 |
| IL-12-Fc-235 | p35 D159F | 19 | p40 K260S R261S | 66 |
| IL-12-Fc-235 | p35 Y161S | 20 | p40 K260S R261S | 66 |
| IL-12-Fc-237 | p35 Y161T | 21 | p40 K260S R261S | 66 |
| IL-12-Fc-238 | p35 Y161A | 22 | p40 K260S R261S | 66 |
| IL-12-Fc-239 | p35 Y161W | 23 | p40 K260S R261S | 66 |
| IL-12-Fc-240 | p35 K162E | 24 | p40 K260S R261S | 66 |
| IL-12-Fc-241 | p35 K162D | 25 | p40 K260S R261S | 66 |

IL-12p35 subunits have N-terminal hexapeptide truncation

In particular embodiments of the present invention, the monovalent IL-12 heterodimeric Fc protein comprises a heterodimer of an IL-12p35-Fc (LALADS-KIH-Knob) and an IL-12p40-Fc (LALADS-KIH-Hole), wherein the IL-12p35-Fc (LALADS-KIH-Knob) comprises the amino acid sequence set forth in SEQ ID NO: 8 and the IL-12p40-Fc (LALADS-KIH-Hole) comprises the amino acid sequence set forth in SEQ ID NO: 2.

In particular embodiments of the present invention, the monovalent IL-12 heterodimeric Fc protein comprises a heterodimer of an IL-12p35-Fc (LALADS-KIH-Knob) and an IL-12p40-Fc (LALADS-KIH-Hole), wherein the IL-12p35-Fc (LALADS-KIH-Knob) comprises the amino acid sequence set forth in SEQ ID NO: 15 and the IL-12p40-Fc (LALADS-KIH-Hole) comprises the amino acid sequence set forth in SEQ ID NO: 2.

In particular embodiments of the present invention, the monovalent IL-12 heterodimeric Fc protein comprises a heterodimer of an IL-12p35-Fc (LALADS-KIH-Knob) and an IL-12p40-Fc (LALADS-KIH-Hole), wherein the IL-12p35-Fc (LALADS-KIH-Knob) comprises the amino acid sequence set forth in SEQ ID NO: 16 and the IL-12p40-Fc (LALADS-KIH-Hole) comprises the amino acid sequence set forth in SEQ ID NO: 2.

In particular embodiments of the present invention, the monovalent IL-12 heterodimeric Fc protein comprises a heterodimer of an IL-12p35-Fc (LALADS-KIH-Knob) and an IL-12p40-Fc (LALADS-KIH-Hole), wherein the IL-12p35-Fc (LALADS-KIH-Knob) comprises the amino acid sequence set forth in SEQ ID NO: 17 and the IL-12p40-Fc (LALADS-KIH-Hole) comprises the amino acid sequence set forth in SEQ ID NO: 2.

In particular embodiments of the present invention, the monovalent IL-12 heterodimeric Fc protein comprises a heterodimer of an IL-12p35-Fc (LALADS-KIH-Knob) and an IL-12p40-Fc (LALADS-KIH-Hole), wherein the IL-12p35-Fc (LALADS-KIH-Knob) comprises the amino acid sequence set forth in SEQ ID NO: 8 and the IL-12p40-Fc (LALADS-KIH-Hole) comprises the amino acid sequence set forth in SEQ ID NO: 2.

In particular embodiments of the present invention, the monovalent IL-12 heterodimeric Fc protein comprises a heterodimer of an IL-12p35-Fc (LALADS-KIH-Knob) and an IL-12p40-Fc (LALADS-KIH-Hole), wherein the IL-12p35-Fc (LALADS-KIH-Knob) comprises the amino acid sequence set forth in SEQ ID NO: 20 and the IL-12p40-Fc (LALADS-KIH-Hole) comprises the amino acid sequence set forth in SEQ ID NO: 2.

In particular embodiments of the present invention, the monovalent IL-12 heterodimeric Fc protein comprises a heterodimer of an IL-12p35-Fc (LALADS-KIH-Knob) and an IL-12p40-Fc (LALADS-KIH-Hole), wherein the IL-12p35-Fc (LALADS-KIH-Knob) comprises the amino acid sequence set forth in SEQ ID NO: 21 and the IL-12p40-Fc (LALADS-KIH-Hole) comprises the amino acid sequence set forth in SEQ ID NO: 2.

In particular embodiments of the present invention, the monovalent IL-12 heterodimeric Fc protein comprises a heterodimer of an IL-12p35-Fc (LALADS-KIH-Knob) and an IL-12p40-Fc (LALADS-KIH-Hole), wherein the IL-12p35-Fc (LALADS-KIH-Knob) comprises the amino acid sequence set forth in SEQ ID NO: 22 and the IL-12p40-Fc (LALADS-KIH-Hole) comprises the amino acid sequence set forth in SEQ ID NO: 2.

In particular embodiments of the present invention, the monovalent IL-12 heterodimeric Fc protein comprises a heterodimer of an IL-12p35-Fc (LALADS-KIH-Knob) and an IL-12p40-Fc (LALADS-KIH-Hole), wherein the IL-12p35-Fc (LALADS-KIH-Knob) comprises the amino acid sequence set forth in SEQ ID NO: 8 and the IL-12p40-Fc (LALADS-KIH-Hole) comprises the amino acid sequence set forth in SEQ ID NO: 60, SEQ ID NO: 61, SEQ ID NO: 62, SEQ ID NO: 63, SEQ ID NO: 64, SEQ ID NO: 65, or SEQ ID NO: 66.

In particular embodiments of the present invention, the monovalent IL-12 heterodimeric Fc protein comprises a heterodimer of an IL-12p35-Fc (LALADS-KIH-Knob) and an IL-12p40-Fe (LALADS-KIH-Hole), wherein the IL-12p35-Fc (LALADS-KIH-Knob) comprises the amino acid sequence set forth in SEQ ID NO: 15 and the IL-12p40-Fc (LALADS-KIH-Hole) comprises the amino acid sequence set forth in SEQ ID NO: 60, SEQ ID NO: 61, SEQ ID NO: 62, SEQ ID NO: 63, SEQ ID NO: 64, SEQ ID NO: 65, or SEQ ID NO: 66.

In particular embodiments of the present invention, the monovalent IL-12 heterodimeric Fc protein comprises a heterodimer of an IL-12p35-Fc (LALADS-KIH-Knob) and an IL-12p40-Fc (LALADS-KIH-Hole), wherein the IL-12p35-Fc (LALADS-KIH-Knob) comprises the amino acid sequence set forth in SEQ ID NO: 16 and the IL-12p40-Fc (LALADS-KIH-Hole) comprises the amino acid sequence set forth in SEQ ID NO: 60, SEQ ID NO: 61, SEQ ID NO: 62, SEQ ID NO: 63, SEQ ID NO: 64, SEQ ID NO: 65, or SEQ ID NO: 66.

In particular embodiments of the present invention, the monovalent IL-12 heterodimeric Fc protein comprises a heterodimer of an IL-12p35-Fc (LALADS-KIH-Knob) and an IL-12p40-Fc (LALADS-KIH-Hole), wherein the IL-12p35-Fc (LALADS-KIH-Knob) comprises the amino acid sequence set forth in SEQ ID NO: 17 and the IL-12p40-Fc (LALADS-KIH-Hole) comprises the amino acid sequence set forth in SEQ ID NO: 60, SEQ ID NO: 61, SEQ ID NO: 62, SEQ ID NO: 63, SEQ ID NO: 64, SEQ ID NO: 65, or SEQ ID NO: 66.

In particular embodiments of the present invention, the monovalent IL-12 heterodimeric Fc protein comprises a heterodimer of an IL-12p35-Fc (LALADS-KIH-Knob) and an IL-12p40-Fc (LALADS-KIH-Hole), wherein the IL-12p35-Fc (LALADS-KIH-Knob) comprises the amino acid sequence set forth in SEQ ID NO: 20 and the IL-12p40-Fc (LALADS-KIH-Hole) comprises the amino acid sequence set forth in SEQ ID NO: 60, SEQ ID NO: 61, SEQ ID NO: 62, SEQ ID NO: 63, SEQ ID NO: 64, SEQ ID NO: 65, or SEQ ID NO: 66.

In particular embodiments of the present invention, the monovalent IL-12 heterodimeric Fc protein comprises a heterodimer of an IL-12p35-Fc (LALADS-KIH-Knob) and an IL-12p40-Fc (LALADS-KIH-Hole), wherein the IL-12p35-Fc (LALADS-KIH-Knob) comprises the amino acid sequence set forth in SEQ ID NO: 21 and the IL-12p40-Fc (LALADS-KIH-Hole) comprises the amino acid sequence set forth in SEQ ID NO: 60, SEQ ID NO: 61, SEQ ID NO: 62, SEQ ID NO: 63, SEQ ID NO: 64, SEQ ID NO: 65, or SEQ ID NO: 66.

In particular embodiments of the present invention, the monovalent IL-12 heterodimeric Fc protein comprises a heterodimer of an IL-12p35-Fc (LALADS-KIH-Knob) and an IL-12p40-Fc (LALADS-KIH-Hole), wherein the IL-12p35-Fc (LALADS-KIH-Knob) comprises the amino acid sequence set forth in SEQ ID NO: 22 and the IL-12p40-Fc (LALADS-KIH-Hole) comprises the amino acid sequence set forth in SEQ ID NO: 60, SEQ ID NO: 61, SEQ ID NO: 62, SEQ ID NO: 63, SEQ ID NO: 64, SEQ ID NO: 65, or SEQ ID NO: 66.

In particular embodiments of the present invention, the monovalent IL-12 heterodimeric Fc protein comprises a heterodimer of an IL-12p35-Fc (LALADS-KIH-Knob) and an IL-12p40-Fc (LALADS-KIH-Hole), wherein the IL-12p35-Fc (LALADS-KIH-Knob) comprises the amino acid sequence set forth in SEQ ID NO: 1 and the IL-12p40-Fc (LALADS-KIH-Hole) comprises the amino acid sequence set forth in SEQ ID NO: 29. In a further embodiment, the IL-12p40 subunit further comprises a P143A-P215A, P143A-P215A-K260A-R261S-V303I, P143A-P215A-K260S-R261S-V303I, K260A-R261S-V303I, K260S-R261S-V303I, K260A-R261S, or K260S-R261S amino acid substitution.

In particular embodiments of the present invention, the monovalent IL-12 heterodimeric Fc protein comprises a heterodimer of an IL-12p35-Fc (LALADS-KIH-Knob) and an IL-12p40-Fc (LALADS-KIH-Hole), wherein the IL-12p35-Fc (LALADS-KIH-Knob) comprises the amino acid sequence set forth in SEQ ID NO: 1 and the IL-12p40-Fc (LALADS-KIH-Hole) comprises the amino acid sequence set forth in SEQ ID NO: 34. In a further embodiment, the IL-12p40 subunit further comprises a P143A-P215A, P143A-P215A-K260A-R261S-V303I, P143A-P215A-K260S-R261S-V303I, K260A-R261S-V303I, K260S-R261S-V303I, K260A-R261S, or K260S-R261S amino acid substitution.

In particular embodiments of the present invention, the monovalent IL-12 heterodimeric Fc protein comprises a heterodimer of an IL-12p35-Fc (LALADS-KIH-Knob) and an IL-12p40-Fc (LALADS-KIH-Hole), wherein the IL-12p35-Fc (LALADS-KIH-Knob) comprises the amino acid sequence set forth in SEQ ID NO: 1 and the IL-12p40-Fc (LALADS-KIH-Hole) comprises the amino acid sequence set forth in SEQ ID NO: 35. In a further embodiment, the IL-12p40 subunit further comprises a P143A-P215A, P143A-P215A-K260A-R261S-V303I, P143A-P215A-K260S-R261S-V303I, K260A-R261S-V303I, K260S-R261S-V303I, K260A-R261S, or K260S-R261S amino acid substitution.

In particular embodiments of the present invention, the monovalent IL-12 heterodimeric Fc protein comprises a heterodimer of an IL-12p35-Fc (LALADS-KIH-Knob) and an IL-12p40-Fc (LALADS-KIH-Hole), wherein the IL-12p35-Fc (LALADS-KIH-Knob) comprises the amino acid sequence set forth in SEQ ID NO: 20 and the IL-12p40-Fc (LALADS-KIH-Hole) comprises the amino acid sequence set forth in SEQ ID NO: 29. In a further embodiment, the IL-12p40 subunit further comprises a P143A-P215A, P143A-P215A-K260A-R261S-V303I, P143A-P215A-K260S-R261S-V303I, K260A-R261S-V303I, K260S-R261S-V303I, K260A-R261S, or K260S-R261S amino acid substitution.

In particular embodiments of the present invention, the monovalent IL-12 heterodimeric Fc protein comprises a heterodimer of an IL-12p35-Fc (LALADS-KIH-Knob) and an IL-12p40-Fe (LALADS-KIH-Hole), wherein the IL-12p35-Fc (LALADS-KIH-Knob) comprises the amino acid sequence set forth in SEQ ID NO: 20 and the IL-12p40-Fc (LALADS-KIH-Hole) comprises the amino acid sequence set forth in SEQ ID NO: 34. In a further embodiment, the IL-12p40 subunit further comprises a P143A-P215A, P143A-P215A-K260A-R261S-V303I, P143A-P215A-K260S-R261S-V303I, K260A-R261S-V303I, K260S-R261S-V303I, K260A-R261S, or K260S-R261S amino acid substitution.

In particular embodiments of the present invention, the monovalent IL-12 heterodimeric Fc protein comprises a heterodimer of an IL-12p35-Fc (LALADS-KIH-Knob) and an IL-12p40-Fc (LALADS-KIH-Hole), wherein the IL-12p35-Fc (LALADS-KIH-Knob) comprises the amino acid sequence set forth in SEQ ID NO: 20 and the IL-12p40-Fc (LALADS-KIH-Hole) comprises the amino acid sequence set forth in SEQ ID NO: 35. In a further embodiment, the IL-12p40 subunit further comprises a P143A-P215A, P143A-P215A-K260A-R261S-V303I, P143A-P215A-K260S-R261S-V303I, K260A-R261S-V303I, K260S-R261S-V303I, K260A-R261S, or K260S-R261S amino acid substitution.

In particular embodiments of the present invention, the monovalent IL-12 heterodimeric Fc protein comprises a heterodimer of an IL-12p35-Fc (LALADS-KIH-Knob) and an IL-12p40-Fc (LALADS-KIH-Hole), wherein the IL-12p35-Fc (LALADS-KIH-Knob) comprises the amino acid sequence set forth in SEQ ID NO: 21 and the IL-12p40-Fc (LALADS-KIH-Hole) comprises the amino acid sequence set forth in SEQ ID NO: 29. In a further embodiment, the IL-12p40 subunit further comprises a P143A-P215A, P143A-P215A-K260A-R261S-V303I, P143A-P215A-K260S-R261S-V303I, K260A-R261S-V303I, K260S-R261S-V303I, K260A-R261S, or K260S-R261S amino acid substitution.

In particular embodiments of the present invention, the monovalent IL-12 heterodimeric Fc protein comprises a heterodimer of an IL-12p35-Fc (LALADS-KIH-Knob) and an IL-12p40-Fc (LALADS-KIH-Hole), wherein the IL-12p35-Fc (LALADS-KIH-Knob) comprises the amino acid sequence set forth in SEQ ID NO: 21 and the IL-12p40-Fc (LALADS-KIH-Hole) comprises the amino acid sequence set forth in SEQ ID NO: 34. In a further embodiment, the IL-12p40 subunit further comprises a P143A-P215A, P143A-P215A-K260A-R261S-V303I, P143A-P215A-K260S-R261S-V303I, K260A-R261S-V303I, K260S-R261S-V303I, K260A-R261S, or K260S-R261S amino acid substitution.

In particular embodiments of the present invention, the monovalent IL-12 heterodimeric Fc protein comprises a heterodimer of an IL-12p35-Fc (LALADS-KIH-Knob) and an IL-12p40-Fe (LALADS-KIH-Hole), wherein the IL-12p35-Fc (LALADS-KIH-Knob) comprises the amino acid sequence set forth in SEQ ID NO: 21 and the IL-12p40-Fc (LALADS-KIH-Hole) comprises the amino acid sequence set forth in SEQ ID NO: 35. In a further embodiment, the IL-12p40 subunit further comprises a P143A-P215A, P143A-P215A-K260A-R261S-V303I, P143A-P215A-K260S-R261S-V303I, K260A-R261S-V303I, K260S-R261S-V303I, K260A-R261S, or K260S-R261S amino acid substitution.

### III.(e): Monovalent IL-12 Heterodimeric Fc Protein Further Comprising Fc Region Substitutions for Extending Half-Life

In further embodiments of the monovalent IL-12 heterodimeric Fc protein, the first and second Fc regions further comprise a substitution of the amino acids that confer a longer circulation half-life by virtue of more favorable binding to hFcRn. Fc domains containing the amino acid substitutions M428L/N434S (LS mutant), L309D/Q311H/N434S (DHS mutant), H433K/N434F (KF mutant), or M252Y/S254T/T256E (YTE mutant) confer 10- to 12-fold higher affinity for FcRn at pH 5.8, result in the greatest reported increase in antibody half-life (2- to 4-fold in circulation) in mice and in non-human primates (See Lee et al., Nat. Communications 10, 5031 (2019); Fc positions according to Eu numbering scheme). Thus, the present invention includes embodiments that comprises any one of these Half-life extension amino acid substitutions.

In particular embodiments of the monovalent IL-12 heterodimeric Fc protein, the first and second Fc regions further comprise a substitution of the amino acids at positions 428 and 434 of the constant domain of the heavy chain with amino acids Leu (L) and Ser (S), respectively (M428L/N434S substitution) wherein the numbering is according to Eu Index, to provide a heavy chain constant domain comprising a “LS” substitution and having a serum half-life longer than that of an Fc region not so modified.

In particular embodiments of the monovalent IL-12 heterodimeric Fc protein, the first and second Fc regions further comprise a substitution of the amino acids at positions 309, 311, and 434 of the constant domain of the heavy chain with amino acids Asp (D), His (H), and Ser (S), respectively (L309D/Q311H/N434S substitution) wherein the numbering is according to Eu Index, to provide a heavy chain constant domain comprising a “DHS” substitution and having a serum half-life longer than that of an Fc region not so modified.

In particular embodiments of the monovalent IL-12 heterodimeric Fc protein, the first and second Fc regions further comprise a substitution of the amino acids at positions 433 and 434 of the constant domain of the heavy chain with amino acids Lys (K) and Phe (F), respectively (H433K/N434F substitution) wherein the numbering is according to Eu Index, to provide a heavy chain constant domain comprising a “KF” substitution and having a serum half-life longer than that of an Fc region not so modified.

In particular embodiments of the monovalent IL-12 heterodimeric Fc protein, the first and second Fc regions further comprise a substitution of the amino acids at positions 252, 254, and 256 of the constant domain of the heavy chain with amino acids Tyr (Y), Thr (T), and Glu (E), respectively (M252Y, S254T, T256E substitution) wherein the numbering is according to Eu Index, to provide a heavy chain constant domain comprising a “YTE” substitution and having a serum half-life longer than that of an Fc region not so modified.

In particular embodiments of the monovalent IL-12 heterodimeric Fc protein, the first Fc region is a LALADS-LS-KIH-Knob mutant comprising SEQ ID NO: 396 and the second Fc region is a LALADS-LS-KIH-Hole comprising SEQ ID NO: 397. In particular embodiments of the monovalent IL-12 heterodimeric Fc protein, the first Fc region is a LALADS-LS-KIH-Knob mutant comprising SEQ ID NO: 398 and the second Fc region is a LALADS-LS-KIH-Hole comprising SEQ ID NO: 399. In particular embodiments of the monovalent IL-12 heterodimeric Fc protein, the first Fc region is a LALADS-LS-KIH-Knob mutant comprising SEQ ID NO: 400 and the second Fc region is a LALADS-LS-KIH-Hole comprising SEQ ID NO: 401. In particular embodiments of the monovalent IL-12 heterodimeric Fc protein comprising the LS mutation, the IL-12p35 Fc region protein comprises an amino acid sequence set forth in SEQ ID NO: 69-91 or 104-123 and the IL-12p40 Fc region protein comprises an a min acid sequence set forth in SEQ ID NO: 92-103 or 124-132.

In particular embodiments of the monovalent IL-12 heterodimeric Fc protein, the first Fc region is a LALADS-DHS-KIH-Knob mutant comprising SEQ ID NO: 402 and the second Fc region is a LALADS-DHS-KIH-Hole comprising SEQ ID NO: 403. In particular embodiments of the monovalent IL-12 heterodimeric Fc protein, the first Fc region is a LALADS-DHS-KIH-Knob mutant comprising SEQ ID NO: 404 and the second Fc region is a LALADS-DHS-KIH-Hole comprising SEQ ID NO: 405. In particular embodiments of the monovalent IL-12 heterodimeric Fc protein, the first Fc region is a LALADS-DHS-KIH-Knob mutant comprising SEQ ID NO: 406 and the second Fc region is a LALADS-DHS-KIH-Hole comprising SEQ ID NO: 407. In particular embodiments of the monovalent IL-12 heterodimeric Fc protein comprising the DHS mutation, the IL-12p35 Fc region protein comprises an amino acid sequence set forth in SEQ ID NO: 69-91 or 104-123 and the IL-12p40 Fc region protein comprises an amine acid sequence set forth in SEQ ID NO: 92-103 or 124-132.

In particular embodiments of the monovalent IL-12 heterodimeric Fc protein, the first Fc region is a LALADS-KF-KIH-Knob mutant comprising SEQ ID NO: 408 and the second Fc region is a LALADS-KF-KIH-Hole comprising SEQ ID NO: 409. In particular embodiments of the monovalent IL-12 heterodimeric Fc protein, the first Fc region is a LALADS-KF-KIH-Knob mutant comprising SEQ ID NO: 410 and the second Fc region is a LALADS-KF-KIH-Hole comprising SEQ ID NO: 411. In particular embodiments of the monovalent IL-12 heterodimeric Fc protein, the first Fc region is a LALADS-KF-KIH-Knob mutant comprising SEQ ID NO: 412 and the second Fc region is a LALADS-KF-KIH-Hole comprising SEQ ID NO: 413. In particular embodiments of the monovalent IL-12 heterodimeric Fc protein comprising the KF mutation, the IL-12p35 Fc region protein comprises an amino acid sequence set forth in SEQ ID NO: 69-91 or 104-123 and the IL-12p40 Fc region protein comprises an amin acid sequence set forth in SEQ ID NO: 92-103 or 124-132.

In particular embodiments of the monovalent IL-12 heterodimeric Fc protein, the first Fc region is a LALADS-YTE-KIH-Knob mutant comprising SEQ ID NO: 210 and the second Fc region is a LALADS-YTE-KIH-Hole comprising SEQ ID NO: 211. In particular embodiments of the monovalent IL-12 heterodimeric Fc protein, the first Fc region is a LALADS-YTE-KIH-Knob mutant comprising SEQ ID NO: 214 and the second Fc region is a LALADS-YTE-KIH-Hole comprising SEQ ID NO: 215. In particular embodiments of the monovalent IL-12 heterodimeric Fc protein, the first Fc region is a LALADS-YTE-KIH-Knob mutant comprising SEQ ID NO: 218 and the second Fc region is a LALADS-YTE-KIH-Hole comprising SEQ ID NO: 219. In particular embodiments of the monovalent IL-12 heterodimeric Fc protein comprising the YTE mutation, the IL-12p35 Fc region protein comprises an amino acid sequence set forth in SEQ ID NO: 69-91 or 104-123 and the IL-12p40 Fc region protein comprises an amino acid sequence set forth in SEQ ID NO: 92-103 or 124-132.

Exemplary IL-12 heterodimeric Fc proteins of the present invention in which the IL-12p35 Fc region protein comprises an Fe (LALADS-YTE-KIH-Knob) and the IL-12p40 Fc region protein comprises an Fe (LALADS-YTE-KIH-Hole) are shown in Tables 16-26.

TABLE 16

| | IL-12p35-Fc (LALADS-YTE-KIH-Knob) | | IL-12p40-Fc (LALADS-YTE-KIH-Hole) | |
|---|---|---|---|---|
| IL-12 Analog | IL-12p35 subunit | SEQ ID NO: | IL-12p40 subunit | SEQ ID NO: |
| IL-12-Fc-242 | p35 E40K | 69 | WT | 68 |
| IL-12-Fc-243 | p35 E40R | 70 | WT | 68 |
| IL-12-Fc-244 | p35 E40W | 71 | WT | 68 |
| IL-12-Fc-245 | p35 141A | 72 | WT | 68 |
| IL-12-Fc-246 | p35 141V | 73 | WT | 68 |
| IL-12-Fc-247 | p35 141S | 74 | WT | 68 |
| IL-12-Fc-248 | p35 141T | 75 | WT | 68 |
| IL-12-Fc-249 | p35 D42A | 76 | WT | 68 |
| IL-12-Fc-250 | p35 D42L | 77 | WT | 68 |
| IL-12-Fc-251 | p35 D42W | 78 | WT | 68 |
| IL-12-Fc-252 | p35 E44A | 79 | WT | 68 |
| IL-12-Fc-253 | p35 E44K | 80 | WT | 68 |
| IL-12-Fc-254 | p35 E44R | 81 | WT | 68 |
| IL-12-Fc-255 | p35 D159K | 82 | WT | 68 |
| IL-12-Fc-256 | p35 D159R | 83 | WT | 68 |
| IL-12-Fc-257 | p35 D159Y | 84 | WT | 68 |
| IL-12-Fc-258 | p35 D159F | 85 | WT | 68 |
| IL-12-Fc-259 | p35 Y161S | 86 | WT | 68 |
| IL-12-Fc-260 | p35 Y161T | 87 | WT | 68 |
| IL-12-Fc-261 | p35 Y161A | 88 | WT | 68 |
| IL-12-Fc-262 | p35 Y161W | 89 | WT | 68 |
| IL-12-Fc-263 | p35 K162E | 90 | WT | 68 |
| IL-12-Fc-264 | p35 K162D | 91 | WT | 68 |

WT = wild-type amino acid sequence
IL-12p35 subunits have N-terminal hexapeptide truncation

TABLE 17

| | p35-Fc (LALADS-YTE-KIH-Knob) | | p40-Fc (LALADS-YTE-KIH-Hole) | |
|---|---|---|---|---|
| IL-12-Analog | IL-12p35 Subunit | SEQ ID NO: | IL-12p40 Subunit | SEQ ID NO: |
| IL-12-Fc-265 | WT | 67 | p40 K85E | 92 |
| IL-12-Fc-266 | WT | 67 | p40 K85Y | 93 |
| IL-12-Fc-267 | WT | 67 | p40 D87A | 94 |
| IL-12-Fc-268 | WT | 67 | p40 D87L | 95 |
| IL-12-Fc-269 | WT | 67 | p40 I89A | 96 |
| IL-12-Fc-270 | WT | 67 | p40 I89S | 97 |
| IL-12-Fc-271 | WT | 67 | p40 I89D | 98 |
| IL-12-Fc-272 | WT | 67 | p40 I89P | 99 |
| IL-12-Fc-273 | WT | 67 | p40 K104E | 100 |
| IL-12-Fc-274 | WT | 67 | p40 K104D | 101 |
| IL-12-Fc-275 | WT | 67 | p40 K104W | 102 |
| IL-12-Fc-276 | WT | 67 | p40 K104S | 103 |

WT = wild-type amino acid sequence
IL-12p35 subunits have N-terminal hexapeptide truncation

TABLE 18

| | p35-Fc (LALADS-YTE-KIH-Knob) | | p40-Fc (LALADS-YTE-KIH-Hole) | |
|---|---|---|---|---|
| IL-12-Analog | IL-12p35 Subunit | SEQ ID NO: | IL-12p40 Subunit | SEQ ID NO: |
| IL-12-Fc-277 | p35 141S | 74 | p40 D87L | 95 |
| IL-12-Fc-278 | p35 E44R | 81 | p40 D87L | 95 |
| IL-12-Fc-279 | p35 D159K | 82 | p40 D87L | 95 |
| IL-12-Fc-280 | p35 D159R | 83 | p40 D87L | 95 |
| IL-12-Fc-281 | p35 Y161S | 86 | p40 D87L | 95 |
| IL-12-Fc-282 | p35 Y161T | 87 | p40 D87L | 95 |
| IL-12-Fc-283 | p35 Y161A | 88 | p40 D87L | 95 |
| IL-12-Fc-284 | p35 141S | 74 | p40 K104E | 100 |
| IL-12-Fc-285 | p35 E44R | 81 | p40 K104E | 100 |
| IL-12-Fc-286 | p35 D159K | 82 | p40 K104E | 100 |
| IL-12-Fc-287 | p35 D159R | 83 | p40 K104E | 100 |
| IL-12-Fc-288 | p35 Y161S | 86 | p40 K104E | 100 |
| IL-12-Fc-289 | p35 Y161T | 87 | p40 K104E | 100 |
| IL-12-Fc-290 | p35 Y161A | 88 | p40 K104E | 100 |
| IL-12-Fc-291 | p35 141S | 74 | p40 K104D | 101 |
| IL-12-Fc-292 | p35 E44R | 81 | p40 K104D | 101 |
| IL-12-Fc-293 | p35 D159K | 82 | p40 K104D | 101 |
| IL-12-Fc-294 | p35 D159R | 83 | p40 K104D | 101 |
| IL-12-Fc-295 | p35 Y161S | 86 | p40 K104D | 101 |
| IL-12-Fc-296 | p35 Y161T | 87 | p40 K104D | 101 |
| IL-12-Fc-297 | p35 Y161A | 88 | p40 K104D | 101 |

IL-12p35 subunits have N-terminal hexapeptide truncation

TABLE 19

| | p35-Fc (LALADS-YTE-KIH-Knob) | | p40-Fc (LALADS-YTE-KIH-Hole) | |
|---|---|---|---|---|
| IL-12-Analog | IL-12p35 Subunit | SEQ ID NO: | IL-12p40 Subunit | SEQ ID NO: |
| IL-12-Fc-298 | p35 Y161T | 87 | p40 D87L K104E | 124 |
| IL-12-Fc-299 | p35 Y161A | 88 | p40 D87L K104D | 125 |
| IL-12-Fc-300 | p35 D159K Y161T | 104 | p40 D87L K104E | 124 |
| IL-12-Fc-301 | p35 D159K Y161T | 104 | p40 D87L K104D | 125 |
| IL-12-Fc-302 | p35 F33G D159K Y161T | 105 | p40 D87L K104E | 124 |
| IL-12-Fc-303 | p35 F33K D159K Y161T | 106 | p40 D87L K104E | 124 |
| IL-12-Fc-304 | p35 F33S D159K Y161T | 107 | p40 D87L K104E | 124 |
| IL-12-Fc-305 | p35 Y34G D159K Y161T | 108 | p40 D87L K104E | 124 |
| IL-12-Fc-306 | p35 Y34S D159K Y161T | 109 | p40 D87L K104E | 124 |
| IL-12-Fc-307 | p35 Y34T D159K Y161T | 110 | p40 D87L K104E | 124 |
| IL-12-Fc-308 | p35 P35Q D159K Y161T | 111 | p40 D87L K104E | 124 |
| IL-12-Fc-309 | p35 P35E D159K Y161T | 112 | p40 D87L K104E | 124 |
| IL-12-Fc-310 | p35 F33S Y34T D159K Y161T | 113 | p40 D87L K104E | 124 |
| IL-12-Fc-311 | p35 F33S Y34S D159K Y161T | 114 | p40 D87L K104E | 124 |
| IL-12-Fc-312 | p35 F33G Y161T | 115 | p40 D87L K104E | 124 |
| IL-12-Fc-313 | p35 F33K Y161T | 116 | p40 D87L K104E | 124 |
| IL-12-Fc-314 | p35 F33S Y161T | 117 | p40 D87L K104E | 124 |
| IL-12-Fc-315 | p35 Y34G Y161T | 118 | p40 D87L K104E | 124 |
| IL-12-Fc-316 | p35 Y34S Y161T | 119 | p40 D87L K104E | 124 |
| IL-12-Fc-317 | p35 Y34T Y161T | 120 | p40 D87L K104E | 124 |
| IL-12-Fc-318 | p35 F33S Y34T Y161T | 121 | p40 D87L K104E | 124 |
| IL-12-Fc-319 | p35 F33S Y34S Y161T | 122 | p40 D87L K104E | 124 |
| IL-12-Fc-320 | p35 F33S Y34G Y161T | 123 | p40 D87L K104E | 124 |

IL-12p35 subunits have N-terminal hexapeptide truncation

TABLE 20

| | p35-Fc (LALADS-YTE-KIH-Knob) | | p40-Fc (LALADS-YTE-KIH-Hole) | |
|---|---|---|---|---|
| IL-12-Analog | IL-12p35 Subunit | SEQ ID NO: | IL-12p40 Subunit | SEQ ID NO: |
| IL-12-Fc-321 | p35 E40K | 69 | p40 143A P215A | 126 |
| IL-12-Fc-322 | p35 E40R | 70 | p40 143A P215A | 126 |
| IL-12-Fc-323 | p35 E40W | 71 | p40 143A P215A | 126 |
| IL-12-Fc-324 | p35 141A | 72 | p40 143A P215A | 126 |
| IL-12-Fc-325 | p35 141V | 73 | p40 143A P215A | 126 |
| IL-12-Fc-326 | p35 141S | 74 | p40 143A P215A | 126 |
| IL-12-Fc-327 | p35 I41T | 75 | p40 143A P215A | 126 |
| IL-12-Fc-328 | p35 D42A | 76 | p40 143A P215A | 126 |
| IL-12-Fc-329 | p35 D42L | 77 | p40 143A P215A | 126 |
| IL-12-Fc-330 | p35 D42W | 78 | p40 143A P215A | 126 |
| IL-12-Fc-331 | p35 E44A | 79 | p40 143A P215A | 126 |
| IL-12-Fc-332 | p35 E44K | 80 | p40 143A P215A | 126 |
| IL-12-Fc-333 | p35 E44R | 81 | p40 143A P215A | 126 |
| IL-12-Fc-334 | p35 D159K | 82 | p40 143A P215A | 126 |
| IL-12-Fc-335 | p35 D159R | 83 | p40 143A P215A | 126 |
| IL-12-Fc-336 | p35 D159Y | 84 | p40 143A P215A | 126 |
| IL-12-Fc-337 | p35 D159F | 85 | p40 143A P215A | 126 |
| IL-12-Fc-338 | p35 Y161S | 86 | p40 143A P215A | 126 |
| IL-12-Fc-339 | p35 Y161T | 87 | p40 143A P215A | 126 |
| IL-12-Fc-340 | p35 Y161A | 88 | p40 143A P215A | 126 |
| IL-12-Fc-341 | p35 Y161W | 89 | p40 143A P215A | 126 |
| IL-12-Fc-342 | p35 K162E | 90 | p40 143A P215A | 126 |
| IL-12-Fc-343 | p35 K162D | 91 | p40 143A P215A | 126 |

IL-12p35 subunits have N-terminal hexapeptide truncation

TABLE 21

| | p35-Fc (LALADS-YTE-KIH-Knob) | | p40-Fc (LALADS-YTE-KIH-Hole) | |
|---|---|---|---|---|
| IL-12-Analog | IL-12p35 Subunit | SEQ ID NO: | IL-12p40 Subunit | SEQ ID NO: |
| IL-12-Fc-344 | p35 E40K | 69 | p40 P143A P215A K260A R261S V303I | 127 |
| IL-12-Fc-345 | p35 E40R | 70 | p40 P143A P215A K260A R261S V303I | 127 |
| IL-12-Fc-346 | p35 E40W | 71 | p40 P143A P215A K260A R261S V303I | 127 |
| IL-12-Fc-347 | p35 I41A | 72 | p40 P143A P215A K260A R261S V303I | 127 |
| IL-12-Fc-348 | p35 141V | 73 | p40 P143A P215A K260A R261S V303I | 127 |
| IL-12-Fc-349 | p35 141S | 74 | p40 P143A P215A K260A R261S V303I | 127 |
| IL-12-Fc-350 | p35 141T | 75 | p40 P143A P215A K260A R261S V303I | 127 |
| IL-12-Fc-351 | p35 D42A | 76 | p40 P143A P215A K260A R261S V303I | 127 |
| IL-12-Fc-352 | p35 D42L | 77 | p40 P143A P215A K260A R261S V303I | 127 |
| IL-12-Fc-353 | p35 D42W | 78 | p40 P143A P215A K260A R261S V303I | 127 |
| IL-12-Fc-354 | p35 E44A | 79 | p40 P143A P215A K260A R261S V303I | 127 |
| IL-12-Fc-355 | p35 E44K | 80 | p40 P143A P215A K260A R261S V303I | 127 |
| IL-12-Fc-356 | p35 E44R | 81 | p40 P143A P215A K260A R261S V303I | 127 |
| IL-12-Fc-357 | p35 D159K | 82 | p40 P143A P215A K260A R261S V303I | 127 |
| IL-12-Fc-358 | p35 D159R | 83 | p40 P143A P215A K260A R261S V303I | 127 |
| IL-12-Fc-359 | p35 D159Y | 84 | p40 P143A P215A K260A R261S V303I | 127 |
| IL-12-Fc-360 | p35 D159F | 85 | p40 P143A P215A K260A R261S V303I | 127 |
| IL-12-Fc-361 | p35 Y161S | 86 | p40 P143A P215A K260A R261S V303I | 127 |
| IL-12-Fc-362 | p35 Y161T | 87 | p40 P143A P215A K260A R261S V303I | 127 |
| IL-12-Fc-363 | p35 Y161A | 88 | p40 P143A P215A K260A R261S V303I | 127 |
| IL-12-Fc-364 | p35 Y161W | 89 | p40 P143A P215A K260A R261S V303I | 127 |
| IL-12-Fc-365 | p35 K162E | 90 | p40 P143A P215A K260A R261S V303I | 127 |
| IL-12-Fc-366 | p35 K162D | 91 | p40 P143A P215A K260A R261S V303I | 127 |

IL-12p35 subunits have N-terminal hexapeptide truncation

TABLE 22

| | p35-Fc (LALADS-YTE-KIH-Knob) | | p40-Fc (LALADS-YTE-KIH-Hole) | |
|---|---|---|---|---|
| IL-12-Analog | IL-12p35 Subunit | SEQ ID NO: | IL-12p40 Subunit | SEQ ID NO: |
| IL-12-Fc-367 | p35 E40K | 69 | p40 P143A P215A K260S R261S V303I | 128 |
| IL-12-Fc-368 | p35 E40R | 70 | p40 P143A P215A K260S R261S V303I | 128 |
| IL-12-Fc-369 | p35 E40W | 71 | p40 P143A P215A K260S R261S V303I | 128 |
| IL-12-Fc-370 | p35 141A | 72 | p40 P143A P215A K260S R261S V303I | 128 |
| IL-12-Fc-371 | p35 141V | 73 | p40 P143A P215A K260S R261S V303I | 128 |
| IL-12-Fc-372 | p35 141S | 74 | p40 P143A P215A K260S R261S V303I | 128 |
| IL-12-Fc-373 | p35 141T | 75 | p40 P143A P215A K260S R261S V303I | 128 |
| IL-12-Fc-374 | p35 D42A | 76 | p40 P143A P215A K260S R261S V303I | 128 |
| IL-12-Fc-375 | p35 D42L | 77 | p40 P143A P215A K260S R261S V303I | 128 |
| IL-12-Fc-376 | p35 D42W | 78 | p40 P143A P215A K260S R261S V303I | 128 |
| IL-12-Fc-377 | p35 E44A | 79 | p40 P143A P215A K260S R261S V303I | 128 |
| IL-12-Fc-378 | p35 E44K | 80 | p40 P143A P215A K260S R261S V303I | 128 |
| IL-12-Fc-379 | p35 E44R | 81 | p40 P143A P215A K260S R261S V303I | 128 |
| IL-12-Fc-380 | p35 D159K | 82 | p40 P143A P215A K260S R261S V303I | 128 |
| IL-12-Fc-381 | p35 D159R | 83 | p40 P143A P215A K260S R261S V303I | 128 |
| IL-12-Fc-382 | p35 D159Y | 84 | p40 P143A P215A K260S R261S V303I | 128 |
| IL-12-Fc-380 | p35 D159F | 85 | p40 P143A P215A K260S R261S V303I | 128 |
| IL-12-Fc-383 | p35 Y161S | 86 | p40 P143A P215A K260S R261S V303I | 128 |
| IL-12-Fc-384 | p35 Y161T | 87 | p40 P143A P215A K260S R261S V303I | 128 |
| IL-12-Fc-385 | p35 Y161A | 88 | p40 P143A P215A K260S R261S V303I | 128 |
| IL-12-Fc-386 | p35 Y161W | 89 | p40 P143A P215A K260S R261S V303I | 128 |
| IL-12-Fc-387 | p35 K162E | 90 | p40 P143A P215A K260S R261S V303I | 128 |
| IL-12-Fc-388 | p35 K162D | 91 | p40 P143A P215A K260S R261S V303I | 128 |

IL-12p35 subunits have N-terminal hexapeptide truncation

TABLE 23

| | p35-Fc (LALADS-YTE-KIH-Knob) | | p40-Fc (LALADS-YTE-KIH-Hole) | |
|---|---|---|---|---|
| IL-12-Analog | IL-12p35 Subunit | SEQ ID NO: | IL-12p40 Subunit | SEQ ID NO: |
| IL-12-Fc-389 | p35 E40K | 69 | p40 K260A R261S V303I | 129 |
| IL-12-Fc-390 | p35 E40R | 70 | p40 K260A R261S V303I | 129 |
| IL-12-Fc-391 | p35 E40W | 71 | p40 K260A R261S V303I | 129 |
| IL-12-Fc-392 | p35 I41A | 72 | p40 K260A R261S V303I | 129 |
| IL-12-Fc-393 | p35 141V | 73 | p40 K260A R261S V303I | 129 |
| IL-12-Fc-394 | p35 141S | 74 | p40 K260A R261S V303I | 129 |
| IL-12-Fc-395 | p35 141T | 75 | p40 K260A R261S V303I | 129 |
| IL-12-Fc-396 | p35 D42A | 76 | p40 K260A R261S V303I | 129 |
| IL-12-Fc-397 | p35 D42L | 77 | p40 K260A R261S V303I | 129 |
| IL-12-Fc-398 | p35 D42W | 78 | p40 K260A R261S V303I | 129 |
| IL-12-Fc-399 | p35 E44A | 79 | p40 K260A R261S V303I | 129 |
| IL-12-Fc-400 | p35 E44K | 80 | p40 K260A R261S V303I | 129 |
| IL-12-Fc-401 | p35 E44R | 81 | p40 K260A R261S V303I | 129 |
| IL-12-Fc-402 | p35 D159K | 82 | p40 K260A R261S V303I | 129 |
| IL-12-Fc-403 | p35 D159R | 83 | p40 K260A R261S V303I | 129 |
| IL-12-Fc-404 | p35 D159Y | 84 | p40 K260A R261S V303I | 129 |
| IL-12-Fc-405 | p35 D159F | 85 | p40 K260A R261S V303I | 129 |
| IL-12-Fc-406 | p35 Y161S | 86 | p40 K260A R261S V303I | 129 |
| IL-12-Fc-407 | p35 Y161T | 87 | p40 K260A R261S V303I | 129 |
| IL-12-Fc-408 | p35 Y161A | 88 | p40 K260A R261S V303I | 129 |
| IL-12-Fc-409 | p35 Y161W | 89 | p40 K260A R261S V303I | 129 |
| IL-12-Fc-410 | p35 K162E | 90 | p40 K260A R261S V303I | 129 |
| IL-12-Fc-411 | p35 K162D | 91 | p40 K260A R261S V303I | 129 |

IL-12p35 subunits have N-terminal hexapeptide truncation

TABLE 24

| | p35-Fc (LALADS-YTE-KIH-Knob) | | p40-Fc (LALADS-YTE-KIH-Hole) | |
|---|---|---|---|---|
| IL-12-Analog | IL-12p35 Subunit | SEQ ID NO: | IL-12p40 Subunit | SEQ ID NO: |
| IL-12-Fc-412 | p35 E40K | 69 | p40 K260S R261S V303I | 130 |
| IL-12-Fc-413 | p35 E40R | 70 | p40 K260S R261S V303I | 130 |
| IL-12-Fc-414 | p35 E40W | 71 | p40 K260S R261S V303I | 130 |
| IL-12-Fc-415 | p35 I41A | 72 | p40 K260S R261S V303I | 130 |
| IL-12-Fc-416 | p35 141V | 73 | p40 K260S R261S V303I | 130 |
| IL-12-Fc-417 | p35 141S | 74 | p40 K260S R261S V303I | 130 |
| IL-12-Fc-418 | p35 141T | 75 | p40 K260S R261S V303I | 130 |
| IL-12-Fc-419 | p35 D42A | 76 | p40 K260S R261S V303I | 130 |
| IL-12-Fc-420 | p35 D42L | 77 | p40 K260S R261S V303I | 130 |
| IL-12-Fc-421 | p35 D42W | 78 | p40 K260S R261S V303I | 130 |
| IL-12-Fc-422 | p35 E44A | 79 | p40 K260S R261S V303I | 130 |
| IL-12-Fc-423 | p35 E44K | 80 | p40 K260S R261S V303I | 130 |
| IL-12-Fc-424 | p35 E44R | 81 | p40 K260S R261S V303I | 130 |
| IL-12-Fc-425 | p35 D159K | 82 | p40 K260S R261S V303I | 130 |
| IL-12-Fc-426 | p35 D159R | 83 | p40 K260S R261S V303I | 130 |
| IL-12-Fc-427 | p35 D159Y | 84 | p40 K260S R261S V303I | 130 |
| IL-12-Fc-428 | p35 D159F | 85 | p40 K260S R261S V303I | 130 |
| IL-12-Fc-429 | p35 Y161S | 86 | p40 K260S R261S V303I | 130 |
| IL-12-Fc-430 | p35 Y161T | 87 | p40 K260S R261S V303I | 130 |
| IL-12-Fc-431 | p35 Y161A | 88 | p40 K260S R261S V303I | 130 |
| IL-12-Fc-432 | p35 Y161W | 89 | p40 K260S R261S V303I | 130 |
| IL-12-Fc-433 | p35 K162E | 90 | p40 K260S R261S V303I | 130 |
| IL-12-Fc-434 | p35 K162D | 91 | p40 K260S R261S V303I | 130 |

IL-12p35 subunits have N-terminal hexapeptide truncation

TABLE 25

| | p35-Fc (LALADS-YTE-KIH-Knob) | | p40-Fc (LALADS-YTE-KIH-Hole) | |
|---|---|---|---|---|
| IL-12-Analog | IL-12p35 Subunit | SEQ ID NO: | IL-12p40 Subunit | SEQ ID NO: |
| IL-12-Fc-435 | p35 E40K | 69 | p40 K260A R261S | 131 |
| IL-12-Fc-436 | p35 E40R | 70 | p40 K260A R261S | 131 |
| IL-12-Fc-437 | p35 E40W | 71 | p40 K260A R261S | 131 |
| IL-12-Fc-438 | p35 141A | 72 | p40 K260A R261S | 131 |
| IL-12-Fc-439 | p35 141V | 73 | p40 K260A R261S | 131 |
| IL-12-Fc-440 | p35 I41S | 74 | p40 K260A R261S | 131 |
| IL-12-Fc-441 | p35 I41T | 75 | p40 K260A R261S | 131 |
| IL-12-Fc-442 | p35 D42A | 76 | p40 K260A R261S | 131 |
| IL-12-Fc-443 | p35 D42L | 77 | p40 K260A R261S | 131 |
| IL-12-Fc-444 | p35 D42W | 78 | p40 K260A R261S | 131 |
| IL-12-Fc-445 | p35 E44A | 79 | p40 K260A R261S | 131 |
| IL-12-Fc-446 | p35 E44K | 80 | p40 K260A R261S | 131 |
| IL-12-Fc-447 | p35 E44R | 81 | p40 K260A R261S | 131 |
| IL-12-Fc-448 | p35 D159K | 82 | p40 K260A R261S | 131 |
| IL-12-Fc-449 | p35 D159R | 83 | p40 K260A R261S | 131 |
| IL-12-Fc-450 | p35 D159Y | 84 | p40 K260A R261S | 131 |
| IL-12-Fc-451 | p35 D159F | 85 | p40 K260A R261S | 131 |
| IL-12-Fc-452 | p35 Y161S | 86 | p40 K260A R261S | 131 |
| IL-12-Fc-453 | p35 Y161T | 87 | p40 K260A R261S | 131 |
| IL-12-Fc-454 | p35 Y161A | 88 | p40 K260A R261S | 131 |
| IL-12-Fc-455 | p35 Y161W | 89 | p40 K260A R261S | 131 |
| IL-12-Fc-456 | p35 K162E | 90 | p40 K260A R261S | 131 |
| IL-12-Fc-457 | p35 K162D | 91 | p40 K260A R261S | 131 |

IL-12p35 subunits have N-terminal hexapeptide truncation

TABLE 26

| | p35-Fc (LALADS-YTE-KIH-Knob) | | p40-Fc (LALADS-YTE-KIH-Hole) | |
|---|---|---|---|---|
| IL-12-Analog | IL-12p35 Subunit | SEQ ID NO: | IL-12p40 Subunit | SEQ ID NO: |
| IL-12-Fc-458 | p35 E40K | 69 | p40 K260S R261S | 132 |
| IL-12-Fc-459 | p35 E40R | 70 | p40 K260S R261S | 132 |
| IL-12-Fc-460 | p35 E40W | 71 | p40 K260S R261S | 132 |
| IL-12-Fc-461 | p35 I41A | 72 | p40 K260S R261S | 132 |
| IL-12-Fc-462 | p35 141V | 73 | p40 K260S R261S | 132 |
| IL-12-Fc-463 | p35 141S | 74 | p40 K260S R261S | 132 |
| IL-12-Fc-464 | p35 141T | 75 | p40 K260S R261S | 132 |
| IL-12-Fc-465 | p35 D42A | 76 | p40 K260S R261S | 132 |
| IL-12-Fc-466 | p35 D42L | 77 | p40 K260S R261S | 132 |
| IL-12-Fc-467 | p35 D42W | 78 | p40 K260S R261S | 132 |
| IL-12-Fc-468 | p35 E44A | 79 | p40 K260S R261S | 132 |
| IL-12-Fc-469 | p35 E44K | 80 | p40 K260S R261S | 132 |
| IL-12-Fc-470 | p35 E44R | 81 | p40 K260S R261S | 132 |
| IL-12-Fc-471 | p35 D159K | 82 | p40 K260S R261S | 132 |
| IL-12-Fc-472 | p35 D159R | 83 | p40 K260S R261S | 132 |
| IL-12-Fc-473 | p35 D159Y | 84 | p40 K260S R261S | 132 |
| IL-12-Fc-474 | p35 D159F | 85 | p40 K260S R261S | 132 |
| IL-12-Fc-475 | p35 Y161S | 86 | p40 K260S R261S | 132 |
| IL-12-Fc-476 | p35 Y161T | 87 | p40 K260S R261S | 132 |
| IL-12-Fc-477 | p35 Y161A | 88 | p40 K260S R261S | 132 |
| IL-12-Fc-478 | p35 Y161W | 89 | p40 K260S R261S | 132 |
| IL-12-Fc-479 | p35 K162E | 90 | p40 K260S R261S | 132 |
| IL-12-Fc-480 | p35 K162D | 91 | p40 K260S R261S | 132 |

IL-12p35 subunits have N-terminal hexapeptide truncation

In particular embodiments of the present invention, the monovalent IL-12 heterodimeric Fe protein comprises a heterodimer of an IL-12p35-Fc (LALADS-YTE-KIH-Knob) and an IL-12p40-Fc (LALADS-YTE-KIH-Hole), wherein the IL-12p135-Fe (LALADS-YTE-KIH-Knob) comprises the amino acid sequence set forth in SEQ ID NO: 74 and the IL-12p40-Fc (LALADS-YTE-KIH-Hole) comprises the amino acid sequence set forth in SEQ ID NO: 68.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 81 and the IL-12p40-Fc (LALADS-YTE-KIH-Hole) comprises the amino acid sequence set forth in SEQ ID NO: 68.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 82 and the IL-12p40-Fc (LALADS-YTE-KIH-Hole) comprises the amino acid sequence set forth in SEQ ID NO: 68.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 83 and the IL-12p40-Fc (LALADS-YTE-KIH-Hole) comprises the amino acid sequence set forth in SEQ ID NO: 68.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 86 and the IL-12p40-Fc (LALADS-YTE-KIH-Hole) comprises the amino acid sequence set forth in SEQ ID NO: 68.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 87 and the IL-12p40-Fc (LALADS-YTE-KIH-Hole) comprises the amino acid sequence set forth in SEQ ID NO: 68.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 88 and the IL-12p40-Fc (LALADS-YTE-KIH-Hole) comprises the amino acid sequence set forth in SEQ ID NO: 68.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 74 and the IL-12p40-Fc (LALADS-YTE-KIH-Hole) comprises the amino acid sequence set forth in SEQ ID NO: 126, SEQ ID NO: 127, SEQ ID NO: 128, SEQ ID NO: 129, SEQ ID NO: 130, SEQ ID NO: 131, or SEQ ID NO: 132.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 81 and the IL-12p40-Fc (LALADS-YTE-KIH-Hole) comprises the amino acid sequence set forth in SEQ ID NO: 126, SEQ ID NO: 127, SEQ ID NO: 128, SEQ ID NO: 129, SEQ ID NO: 130, SEQ ID NO: 131, or SEQ ID NO: 132.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 82 and the IL-12p40-Fc (LALADS-YTE-KIH-Hole) comprises the amino acid sequence set forth in SEQ ID NO: 126, SEQ ID NO: 127, SEQ ID NO: 128, SEQ ID NO: 129, SEQ ID NO: 130, SEQ ID NO: 131, or SEQ ID NO: 132.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 83 and the IL-12p40-Fc (LALADS-YTE-KIH-Hole) comprises the amino acid sequence set forth in SEQ ID NO: 126, SEQ ID NO: 127, SEQ ID NO: 128, SEQ ID NO: 129, SEQ ID NO: 130, SEQ ID NO: 131, or SEQ ID NO: 132.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 86 and the IL-12p40-Fc (LALADS-YTE-KIH-Hole) comprises the amino acid sequence set forth in SEQ ID NO: 126, SEQ ID NO: 127, SEQ ID NO: 128, SEQ ID NO: 129, SEQ ID NO: 130, SEQ ID NO: 131, or SEQ ID NO: 132.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 87 and the IL-12p40-Fc (LALADS-YTE-KIH-Hole) comprises the amino acid sequence set forth in SEQ ID NO: 126, SEQ ID NO: 127, SEQ ID NO: 128, SEQ ID NO: 129, SEQ ID NO: 130, SEQ ID NO: 131, or SEQ ID NO: 132.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 88 and the IL-12p40-Fc (LALADS-YTE-KIH-Hole) comprises the amino acid sequence set forth in SEQ ID NO: 126, SEQ ID NO: 127, SEQ ID NO: 128, SEQ ID NO: 129, SEQ ID NO: 130, SEQ ID NO: 131, or SEQ ID NO: 132.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 67 and the IL-12p40-Fc (LALADS-YTE-KIH-Hole) comprises the amino acid sequence set forth in SEQ ID NO: 95. In a further embodiment, the IL-12p40 subunit further comprises a P143A-P215A, P143A-P215A-K260A-R261S-V303I, P143A-P215A-K260S-R261S-V303I, K260A-R261S-V303I, K260S-R261S-V303I, K260A-R261S, or K260S-R261S amino acid substitution.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 67 and the IL-12p40-Fc (LALADS-YTE-KIH-Hole) comprises the amino acid sequence set forth in SEQ ID NO: 100. In a further embodiment, the IL-12p40 subunit further comprises a P143A-P215A, P143A-P215A-K260A-R261S-V303I, P143A-P215A-K260S-R261S-V303I, K260A-R261S-V303I, K260S-R261S-V303I, K260A-R261S, or K260S-R261S amino acid substitution.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 67 and the IL-12p40-Fc (LALADS-YTE-KIH-Hole) comprises the amino acid sequence set forth in SEQ ID NO: 101. In a further embodiment, the IL-12p40 subunit further comprises a P143A-P215A, P143A-P215A-K260A-R261S-V303I, P143A-P215A-K260S-R261S-V303I, K260A-R261S-V303I, K260S-R261S-V303I, K260A-R261S, or K260S-R261S amino acid substitution.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 86 and the IL-12p40-Fc (LALADS-YTE-KIH-Hole) comprises the amino acid sequence set forth in SEQ ID NO: 95. In a further embodiment, the IL-12p40 subunit further comprises a P143A-P215A, P143A-P215A-K260A-R261S-V303I, P143A-P215A-K260S-R261S-V303I, K260A-R261S-V303I, K260S-R261S-V303I, K260A-R261S, or K260S-R261S amino acid substitution.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 86 and the IL-12p40-Fc (LALADS-YTE-KIH-Hole) comprises the amino acid sequence set forth in SEQ ID NO: 100. In a further embodiment, the IL-12p40 subunit further comprises a P143A-P215A, P143A-P215A-K260A-R261S-V303I, P143A-P215A-K260S-R261S-V303I, K260A-R261S-V303I, K260S-R261S-V303I, K260A-R261S, or K260S-R261S amino acid substitution.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 86 and the IL-12p40-Fc (LALADS-YTE-KIH-Hole) comprises the amino acid sequence set forth in SEQ ID NO: 101. In a further embodiment, the IL-12p40 subunit further comprises a P143A-P215A, P143A-P215A-K260A-R261S-V303I, P143A-P215A-K260S-R261S-V303I, K260A-R261S-V303I, K260S-R261S-V303I, K260A-R261S, or K260S-R261S amino acid substitution.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 87 and the IL-12p40-Fc (LALADS-YTE-KIH-Hole) comprises the amino acid sequence set forth in SEQ ID NO: 95. In a further embodiment, the IL-12p40 subunit further comprises a P143A-P215A, P143A-P215A-K260A-R261S-V303I, P143A-P215A-K260S-R261S-V303I, K260A-R261S-V303I, K260S-R261S-V303I, K260A-R261S, or K260S-R261S amino acid substitution.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 87 and the IL-12p40-Fc (LALADS-YTE-KIH-Hole) comprises the amino acid sequence set forth in SEQ ID NO: 100. In a further embodiment, the IL-12p40 subunit further comprises a P143A-P215A, P143A-P215A-K260A-R261S-V303I, P143A-P215A-K260S-R261S-V303I, K260A-R261S-V303I, K260S-R261S-V303I, K260A-R261S, or K260S-R261S amino acid substitution.

In particular embodiments of the present invention, the monovalent IL-12 heterodimeric Fc protein comprises a heterodimer of an IL-12p35-Fc (LALADS-YTE-KIH-Knob) and an IL-12p40-Fc (LALADS-YTE-KIH-Hole), wherein the IL-12p35-Fc (LALADS-YTE-KIH-Knob) comprises the amino acid sequence set forth in SEQ ID NO: 87 and the IL-12p40-Fc (LALADS-YTE-KIH-Hole) comprises the amino acid sequence set forth in SEQ ID NO: 101. In a further embodiment, the IL-12p40 subunit further comprises a P143A-P215A, P143A-P215A-K260A-R261S-V303I, P143A-P215A-K260S-R261S-V303I, K260A-R261S-V303I, K260S-R261S-V303I, K260A-R261S, or K260S-R261S amino acid substitution.

In particular embodiments of the invention, the first and second Fc regions as disclosed herein may comprise a C-terminal lysine or lack either a C-terminal lysine or a C-terminal glycine-lysine dipeptide. Thus, in a composition comprising a particular monovalent IL-12 heterodimeric Fc protein disclosed herein, the composition may comprise a population of molecular species wherein each species may independently comprise a C-terminal lysine, lack a C-terminal lysine, or lack a C-terminal glycine-lysine.

### III.(f): Monovalent IL-12 Heterodimeric Fc Protein Comprising N-Terminal Hexapeptide

The wild-type mature human IL-12p35 subunit comprises the hexapeptide RNLPVA (SEQ ID NO: 238) at the N-terminus. Thus, in particular embodiments, exemplary monovalent IL-12 heterodimeric Fc proteins in which the ILp35 subunit comprises the RNLPVA (SEQ ID NO. 238) hexapeptide at the N-terminus are set forth in Tables 27-37.

TABLE 27

| | IL-12p35-Fc (LALADS-KIH-Knob) | | IL-12p40-Fc (LALADS-KIH-Hole) | |
|---|---|---|---|---|
| IL-12 Analog | IL-12p35 subunit | SEQ ID NO: | IL-12p40 subunit | SEQ ID NO: |
| IL-12-Fc-481 | p35 E40K | 304 | WT | 2 |
| IL-12-Fc-482 | p35 E40R | 305 | WT | 2 |
| IL-12-Fc-483 | p35 E40W | 306 | WT | 2 |
| IL-12-Fc-484 | p35 I41A | 307 | WT | 2 |
| IL-12-Fc-485 | p35 141V | 308 | WT | 2 |
| IL-12-Fc-486 | p35 141S | 309 | WT | 2 |
| IL-12-Fc-487 | p35 141T | 310 | WT | 2 |
| IL-12-Fc-488 | p35 D42A | 311 | WT | 2 |
| IL-12-Fc-489 | p35 D42L | 312 | WT | 2 |
| IL-12-Fc-490 | p35 D42W | 313 | WT | 2 |
| IL-12-Fc-491 | p35 E44A | 314 | WT | 2 |
| IL-12-Fc-492 | p35 E44K | 315 | WT | 2 |
| IL-12-Fc-493 | p35 E44R | 316 | WT | 2 |
| IL-12-Fc-494 | p35 D159K | 317 | WT | 2 |
| IL-12-Fc-495 | p35 D159R | 318 | WT | 2 |
| IL-12-Fc-497 | p35 D159Y | 319 | WT | 2 |
| IL-12-Fc-498 | p35 D159F | 320 | WT | 2 |
| IL-12-Fc-499 | p35 Y161S | 321 | WT | 2 |
| IL-12-Fc-500 | p35 Y161T | 322 | WT | 2 |
| IL-12-Fc-501 | p35 Y161A | 323 | WT | 2 |
| IL-12-Fc-502 | p35 Y161W | 324 | WT | 2 |
| IL-12-Fc-503 | p35 K162E | 325 | WT | 2 |
| IL-12-Fc-504 | p35 K162D | 326 | WT | 2 |

WT = wild-type amino acid sequence
IL-12p35 subunits have N-terminal hexapeptide

TABLE 28

| | p35-Fc (LALADS-KIH-Knob) | | p40-Fc (LALADS-KIH-Hole) | |
|---|---|---|---|---|
| IL-12-Analog | IL-12p35 Subunit | SEQ ID NO: | IL-12p40 Subunit | SEQ ID NO: |
| IL-12-Fc-505 | WT | 389 | p40 K85E | 26 |
| IL-12-Fc-506 | WT | 389 | p40 K85Y | 27 |
| IL-12-Fc-507 | WT | 389 | p40 D87A | 28 |
| IL-12-Fc-508 | WT | 389 | p40 D87L | 29 |
| IL-12-Fc-509 | WT | 389 | p40 I89A | 30 |
| IL-12-Fc-510 | WT | 389 | p40 I89S | 31 |
| IL-12-Fc-511 | WT | 389 | p40 I89D | 32 |
| IL-12-Fc-512 | WT | 389 | p40 189P | 33 |
| IL-12-Fc-513 | WT | 389 | p40 K104E | 34 |
| IL-12-Fc-514 | WT | 389 | p40 K104D | 35 |
| IL-12-Fc-515 | WT | 389 | p40 K104W | 36 |
| IL-12-Fc-516 | WT | 389 | p40 K104S | 37 |

WT = wild-type amino acid sequence
IL-12p35 subunits have N-terminal hexapeptide

TABLE 29

| | p35-Fc (LALADS- | | p40-Fc (LALADS- | |
|---|---|---|---|---|
| | KIH-Knob) | | KIH-Hole) | |
| | IL-12p35 | SEQ ID | IL-12p40 | SEQ ID |
| IL-12-Analog | Subunit | NO: | Subunit | NO: |
| IL-12-Fc-517 | p35 I41S | 309 | p40 D87L | 29 |
| IL-12-Fc-518 | p35 E44R | 316 | p40 D87L | 29 |
| IL-12-Fc-519 | p35 D159K | 317 | p40 D87L | 29 |
| IL-12-Fc-520 | p35 D159R | 318 | p40 D87L | 29 |
| IL-12-Fc-521 | p35 Y161S | 321 | p40 D87L | 29 |
| IL-12-Fc-522 | p35 Y161T | 322 | p40 D87L | 29 |
| IL-12-Fc-523 | p35 Y161A | 323 | p40 D87L | 29 |
| IL-12-Fc-524 | p35 I41S | 309 | p40 K104E | 34 |
| IL-12-Fc-525 | p35 E44R | 316 | p40 K104E | 34 |
| IL-12-Fc-526 | p35 D159K | 317 | p40 K104E | 34 |
| IL-12-Fc-527 | p35 D159R | 318 | p40 K104E | 34 |
| IL-12-Fc-528 | p35 Y161S | 321 | p40 K104E | 34 |
| IL-12-Fc-529 | p35 Y161T | 322 | p40 K104E | 34 |
| IL-12-Fc-530 | p35 Y161A | 323 | p40 K104E | 34 |
| IL-12-Fc-531 | p35 I41S | 309 | p40 K104D | 35 |
| IL-12-Fc-532 | p35 E44R | 316 | p40 K104D | 35 |
| IL-12-Fc-533 | p35 D159K | 317 | p40 K104D | 35 |
| IL-12-Fc-534 | p35 D159R | 318 | p40 K104D | 35 |
| IL-12-Fc-535 | p35 Y161S | 321 | p40 K104D | 35 |
| IL-12-Fc-537 | p35 Y161T | 322 | p40 K104D | 35 |
| IL-12-Fc-538 | p35 Y161A | 323 | p40 K104D | 35 |

IL-12p35 subunits have N-terminal hexapeptide

TABLE 30

| | p35-Fc (LALADS-KIH-Knob) | | p40-Fc (LALADS-KIH Hole) | |
|---|---|---|---|---|
| | | SEQ ID | | SEQ ID |
| IL-12-Analog | IL-12p35 Subunit | NO: | IL-12p40 Subunit | NO: |
| IL-12-Fc-539 | p35 Y161T | 322 | p40 D87L K104E | 34 |
| IL-12-Fc-540 | p35 Y161A | 323 | p40 D87L K104D | 35 |
| IL-12-Fc-541 | p35 D159K Y161T | 327 | p40 D87L K104E | 34 |
| IL-12-Fc-542 | p35 D159K Y161T | 327 | p40 D87L K104D | 35 |
| IL-12-Fc-543 | p35 F33G D159K Y161T | 328 | p40 D87L K104E | 34 |
| IL-12-Fc-544 | p35 F33K D159K Y161T | 329 | p40 D87L K104E | 34 |
| IL-12-Fc-546 | p35 F33S D159K Y161T | 330 | p40 D87L K104E | 34 |
| IL-12-Fc-547 | p35 Y34G D159K Y161T | 331 | p40 D87L K104E | 34 |
| IL-12-Fc-548 | p35 Y34S D159K Y161T | 332 | p40 D87L K104E | 34 |
| IL-12-Fc-549 | p35 Y34T D159K Y161T | 333 | p40 D87L K104E | 34 |
| IL-12-Fc-550 | p35 P35Q D159K Y161T | 334 | p40 D87L K104E | 34 |
| IL-12-Fc-551 | p35 P35E D159K Y161T | 335 | p40 D87L K104E | 34 |
| IL-12-Fc-552 | p35 F33S Y34T D159K | 336 | p40 D87L K104E | 34 |
| | Y161T | | | |
| IL-12-Fc-553 | p35 F33S Y34S D159K | 337 | p40 D87L K104E | 34 |
| | Y161T | | | |
| IL-12-Fc-554 | p35 F33G Y161T | 338 | p40 D87L K104E | 34 |
| IL-12-Fc-555 | p35 F33K Y161T | 339 | p40 D87L K104E | 34 |
| IL-12-Fc-556 | p35 F33S Y161T | 340 | p40 D87L K104E | 34 |
| IL-12-Fc-557 | p35 Y34G Y161T | 341 | p40 D87L K104E | 34 |
| IL-12-Fc-558 | p35 Y34S Y161T | 342 | p40 D87L K104E | 34 |
| IL-12-Fc-559 | p35 Y34T Y161T | 343 | p40 D87L K104E | 34 |
| IL-12-Fc-560 | p35 F33S Y34T Y161T | 344 | p40 D87L K104E | 34 |
| IL-12-Fc-561 | p35 F33S Y34S Y161T | 345 | p40 D87L K104E | 34 |
| IL-12-Fc-562 | p35 F33S Y34G Y161T | 346 | p40 D87L K104E | 34 |

IL-12p35 subunits have N-terminal hexapeptide

TABLE 31

| | p35-Fc (LALADS- | | p40-Fc (LALADS- | |
|---|---|---|---|---|
| | KIH-Knob) | | KIH-Hole) | |
| | IL-12p35 | SEQ ID | IL-12p40 | SEQ ID |
| IL-12-Analog | Subunit | NO: | Subunit | NO: |
| IL-12-Fc-563 | p35 E40K | 304 | p40 143A P215A | 60 |
| IL-12-Fc-564 | p35 E40R | 305 | p40 143A P215A | 60 |
| IL-12-Fc-565 | p35 E40W | 306 | p40 143A P215A | 60 |
| IL-12-Fc-566 | p35 I41A | 307 | p40 143A P215A | 60 |
| IL-12-Fc-567 | p35 I41V | 308 | p40 143A P215A | 60 |
| IL-12-Fc-568 | p35 I41S | 309 | p40 143A P215A | 60 |
| IL-12-Fc-569 | p35 I41T | 310 | p40 143A P215A | 60 |
| IL-12-Fc-570 | p35 D42A | 311 | p40 143A P215A | 60 |
| IL-12-Fc-571 | p35 D42L | 312 | p40 143A P215A | 60 |
| IL-12-Fc-572 | p35 D42W | 313 | p40 143A P215A | 60 |
| IL-12-Fc-573 | p35 E44A | 314 | p40 143A P215A | 60 |
| IL-12-Fc-574 | p35 E44K | 315 | p40 143A P215A | 60 |
| IL-12-Fc-575 | p35 E44R | 316 | p40 143A P215A | 60 |
| IL-12-Fc-576 | p35 D159K | 317 | p40 143A P215A | 60 |
| IL-12-Fc-577 | p35 D159R | 318 | p40 143A P215A | 60 |
| IL-12-Fc-578 | p35 D159Y | 319 | p40 143A P215A | 60 |
| IL-12-Fc-579 | p35 D159F | 320 | p40 143A P215A | 60 |
| IL-12-Fc-580 | p35 Y161S | 321 | p40 143A P215A | 60 |
| IL-12-Fc-581 | p35 Y161T | 322 | p40 143A P215A | 60 |
| IL-12-Fc-582 | p35 Y161A | 323 | p40 143A P215A | 60 |
| IL-12-Fc-583 | p35 Y161W | 324 | p40 143A P215A | 60 |
| IL-12-Fc-584 | p35 K162E | 325 | p40 143A P215A | 60 |
| IL-12-Fc-585 | p35 K162D | 326 | p40 143A P215A | 60 |

IL-12p35 subunits have N-terminal hexapeptide

TABLE 32

| | p35-Fc (LALADS-KIH-Knob) | | p40-Fc (LALADS-KIH-Hole) | |
|---|---|---|---|---|
| | | SEQ ID | | SEQ ID |
| IL-12-Analog | IL-12p35 Subunit | NO: | IL-12p40 Subunit | NO: |
| IL-12-Fc-586 | p35 E40K | 304 | p40 P143A P215A K260A R261S V303I | 61 |
| IL-12-Fc-587 | p35 E40R | 305 | p40 P143A P215A K260A R261S V303I | 61 |
| IL-12-Fc-588 | p35 E40W | 306 | p40 P143A P215A K260A R261S V303I | 61 |
| IL-12-Fc-589 | p35 I41A | 307 | p40 P143A P215A K260A R261S V303I | 61 |
| IL-12-Fc-590 | p35 I41V | 308 | p40 P143A P215A K260A R261S V303I | 61 |
| IL-12-Fc-591 | p35 I41S | 309 | p40 P143A P215A K260A R261S V303I | 61 |
| IL-12-Fc-592 | p35 I41T | 310 | p40 P143A P215A K260A R261S V303I | 61 |
| IL-12-Fc-593 | p35 D42A | 311 | p40 P143A P215A K260A R261S V303I | 61 |
| IL-12-Fc-594 | p35 D42L | 312 | p40 P143A P215A K260A R261S V303I | 61 |
| IL-12-Fc-595 | p35 D42W | 313 | p40 P143A P215A K260A R261S V303I | 61 |
| IL-12-Fc-596 | p35 E44A | 314 | p40 P143A P215A K260A R261S V303I | 61 |
| IL-12-Fc-597 | p35 E44K | 315 | p40 P143A P215A K260A R261S V303I | 61 |
| IL-12-Fc-598 | p35 E44R | 316 | p40 P143A P215A K260A R261S V303I | 61 |
| IL-12-Fc-599 | p35 D159K | 317 | p40 P143A P215A K260A R261S V303I | 61 |
| IL-12-Fc-600 | p35 D159R | 318 | p40 P143A P215A K260A R261S V303I | 61 |
| IL-12-Fc-601 | p35 D159Y | 319 | p40 P143A P215A K260A R261S V303I | 61 |
| IL-12-Fc-602 | p35 D159F | 320 | p40 P143A P215A K260A R261S V303I | 61 |
| IL-12-Fc-603 | p35 Y161S | 321 | p40 P143A P215A K260A R261S V303I | 61 |
| IL-12-Fc-604 | p35 Y161T | 322 | p40 P143A P215A K260A R261S V303I | 61 |
| IL-12-Fc-605 | p35 Y161A | 323 | p40 P143A P215A K260A R261S V303I | 61 |
| IL-12-Fc-606 | p35 Y161W | 324 | p40 P143A P215A K260A R261S V303I | 61 |
| IL-12-Fc-607 | p35 K162E | 325 | p40 P143A P215A K260A R261S V303I | 61 |
| IL-12-Fc-608 | p35 K162D | 326 | p40 P143A P215A K260A R261S V303I | 61 |

IL-12p35 subunits have N-terminal hexapeptide

TABLE 33

| | p35-Fc (LALADS-KIH-Knob) | | p40-Fc (LALADS-KIH-Hole) | |
|---|---|---|---|---|
| | | SEQ ID | | SEQ ID |
| IL-12-Analog | IL-12p35 Subunit | NO: | IL-12p40 Subunit | NO: |
| IL-12-Fc-609 | p35 E40K | 304 | p40 P143A P215A K260S R261S V303I | 62 |
| IL-12-Fc-610 | p35 E40R | 305 | p40 P143A P215A K260S R261S V303I | 62 |
| IL-12-Fc-611 | p35 E40W | 306 | p40 P143A P215A K260S R261S V303I | 62 |
| IL-12-Fc-612 | p35 I41A | 307 | p40 P143A P215A K260S R261S V303I | 62 |
| IL-12-Fc-613 | p35 I41V | 308 | p40 P143A P215A K260S R261S V303I | 62 |
| IL-12-Fc-614 | p35 I41S | 309 | p40 P143A P215A K260S R261S V303I | 62 |
| IL-12-Fc-615 | p35 I41T | 310 | p40 P143A P215A K260S R261S V303I | 62 |
| IL-12-Fc-616 | p35 D42A | 311 | p40 P143A P215A K260S R261S V303I | 62 |
| IL-12-Fc-617 | p35 D42L | 312 | p40 P143A P215A K260S R261S V303I | 62 |
| IL-12-Fc-618 | p35 D42W | 313 | p40 P143A P215A K260S R261S V303I | 62 |
| IL-12-Fc-619 | p35 E44A | 314 | p40 P143A P215A K260S R261S V303I | 62 |
| IL-12-Fc-620 | p35 E44K | 315 | p40 P143A P215A K260S R261S V303I | 62 |
| IL-12-Fc-621 | p35 E44R | 316 | p40 P143A P215A K260S R261S V303I | 62 |
| IL-12-Fc-622 | p35 D159K | 317 | p40 P143A P215A K260S R261S V303I | 62 |
| IL-12-Fc-623 | p35 D159R | 318 | p40 P143A P215A K260S R261S V303I | 62 |
| IL-12-Fc-624 | p35 D159Y | 319 | p40 P143A P215A K260S R261S V303I | 62 |
| IL-12-Fc-625 | p35 D159F | 320 | p40 P143A P215A K260S R261S V303I | 62 |
| IL-12-Fc-626 | p35 Y161S | 321 | p40 P143A P215A K260S R261S V303I | 62 |
| IL-12-Fc-627 | p35 Y161T | 322 | p40 P143A P215A K260S R261S V303I | 62 |
| IL-12-Fc-628 | p35 Y161A | 323 | p40 P143A P215A K260S R261S V303I | 62 |
| IL-12-Fc-629 | p35 Y161W | 324 | p40 P143A P215A K260S R261S V303I | 62 |
| IL-12-Fc-630 | p35 K162E | 325 | p40 P143A P215A K260S R261S V303I | 62 |
| IL-12-Fc-631 | p35 K162D | 326 | p40 P143A P215A K260S R261S V303I | 62 |

IL-12p35 subunits have N-terminal hexapeptide

TABLE 34

| | p35-Fc (LALADS-KIH-Knob) | | p40-Fc (LALADS-KIH-Hole) | |
|---|---|---|---|---|
| | | SEQ ID | | SEQ ID |
| IL-12-Analog | IL-12p35 Subunit | NO: | IL-12p40 Subunit | NO: |
| IL-12-Fc-632 | p35 E40K | 304 | p40 K260A R261S V303I | 63 |
| IL-12-Fc-633 | p35 E40R | 305 | p40 K260A R261S V303I | 63 |
| IL-12-Fc-634 | p35 E40W | 306 | p40 K260A R261S V303I | 63 |
| IL-12-Fc-635 | p35 I41A | 307 | p40 K260A R261S V303I | 63 |
| IL-12-Fc-636 | p35 I41V | 308 | p40 K260A R261S V303I | 63 |
| IL-12-Fc-637 | p35 I41S | 309 | p40 K260A R261S V303I | 63 |
| IL-12-Fc-638 | p35 I41T | 310 | p40 K260A R261S V303I | 63 |
| IL-12-Fc-639 | p35 D42A | 311 | p40 K260A R261S V303I | 63 |
| IL-12-Fc-640 | p35 D42L | 312 | p40 K260A R261S V303I | 63 |
| IL-12-Fc-641 | p35 D42W | 313 | p40 K260A R261S V303I | 63 |
| IL-12-Fc-642 | p35 E44A | 314 | p40 K260A R261S V303I | 63 |
| IL-12-Fc-643 | p35 E44K | 315 | p40 K260A R261S V303I | 63 |
| IL-12-Fc-644 | p35 E44R | 316 | p40 K260A R261S V303I | 63 |
| IL-12-Fc-645 | p35 D159K | 317 | p40 K260A R261S V303I | 63 |
| IL-12-Fc-646 | p35 D159R | 318 | p40 K260A R261S V303I | 63 |
| IL-12-Fc-647 | p35 D159Y | 319 | p40 K260A R261S V303I | 63 |
| IL-12-Fc-648 | p35 D159F | 320 | p40 K260A R261S V303I | 63 |
| IL-12-Fc-649 | p35 Y161S | 321 | p40 K260A R261S V303I | 63 |
| IL-12-Fc-650 | p35 Y161T | 322 | p40 K260A R261S V303I | 63 |
| IL-12-Fc-651 | p35 Y161A | 323 | p40 K260A R261S V303I | 63 |
| IL-12-Fc-652 | p35 Y161W | 324 | p40 K260A R261S V303I | 63 |
| IL-12-Fc-653 | p35 K162E | 325 | p40 K260A R261S V303I | 63 |
| IL-12-Fc-654 | p35 K162D | 326 | p40 K260A R261S V303I | 63 |

IL-12p35 subunits have N-terminal hexapeptide

TABLE 35

| | p35-Fc (LALADS-KIH-Knob) | | p40-Fc (LALADS-KIH-Hole) | |
|---|---|---|---|---|
| | | SEQ ID | | SEQ ID |
| IL-12-Analog | IL-12p35 Subunit | NO: | IL-12p40 Subunit | NO: |
| IL-12-Fc-655 | p35 E40K | 304 | p40 K260S R261S V303I | 64 |
| IL-12-Fc-656 | p35 E40R | 305 | p40 K260S R261S V303I | 64 |
| IL-12-Fc-657 | p35 E40W | 306 | p40 K260S R261S V303I | 64 |
| IL-12-Fc-658 | p35 I41A | 307 | p40 K260S R261S V303I | 64 |
| IL-12-Fc-659 | p35 I41V | 308 | p40 K260S R261S V303I | 64 |
| IL-12-Fc-660 | p35 I41S | 309 | p40 K260S R261S V303I | 64 |
| IL-12-Fc-661 | p35 I41T | 310 | p40 K260S R261S V303I | 64 |
| IL-12-Fc-662 | p35 D42A | 311 | p40 K260S R261S V303I | 64 |
| IL-12-Fc-663 | p35 D42L | 312 | p40 K260S R261S V303I | 64 |
| IL-12-Fc-664 | p35 D42W | 313 | p40 K260S R261S V303I | 64 |
| IL-12-Fc-665 | p35 E44A | 314 | p40 K260S R261S V303I | 64 |
| IL-12-Fc-666 | p35 E44K | 315 | p40 K260S R261S V303I | 64 |
| IL-12-Fc-667 | p35 E44R | 316 | p40 K260S R261S V303I | 64 |
| IL-12-Fc-668 | p35 D159K | 317 | p40 K260S R261S V303I | 64 |
| IL-12-Fc-669 | p35 D159R | 318 | p40 K260S R261S V303I | 64 |
| IL-12-Fc-670 | p35 D159Y | 319 | p40 K260S R261S V303I | 64 |
| IL-12-Fc-671 | p35 D159F | 320 | p40 K260S R261S V303I | 64 |
| IL-12-Fc-672 | p35 Y161S | 321 | p40 K260S R261S V303I | 64 |
| IL-12-Fc-673 | p35 Y161T | 322 | p40 K260S R261S V303I | 64 |
| IL-12-Fc-674 | p35 Y161A | 323 | p40 K260S R261S V303I | 64 |
| IL-12-Fc-675 | p35 Y161W | 324 | p40 K260S R261S V303I | 64 |
| IL-12-Fc-676 | p35 K162E | 325 | p40 K260S R261S V303I | 64 |
| IL-12-Fc-677 | p35 K162D | 326 | p40 K260S R261S V303I | 64 |

IL-12p35 subunits have N-terminal hexapeptide

TABLE 36

| | p35-Fc (LALADS- | | p40-Fc (LALADS- | |
|---|---|---|---|---|
| | KIH-Knob) | | KIH-Hole) | |
| | IL-12p35 | SEQ ID | IL-12p40 | SEQ ID |
| IL-12-Analog | Subunit | NO: | Subunit | NO: |
| IL-12-Fc-678 | p35 E40K | 304 | p40 K260A R261S | 65 |
| IL-12-Fc-679 | p35 E40R | 305 | p40 K260A R261S | 65 |
| IL-12-Fc-680 | p35 E40W | 306 | p40 K260A R261S | 65 |
| IL-12-Fc-681 | p35 I41A | 307 | p40 K260A R261S | 65 |
| IL-12-Fc-682 | p35 I41V | 308 | p40 K260A R261S | 65 |
| IL-12-Fc-683 | p35 I41S | 309 | p40 K260A R261S | 65 |
| IL-12-Fc-684 | p35 I41T | 310 | p40 K260A R261S | 65 |
| IL-12-Fc-685 | p35 D42A | 311 | p40 K260A R261S | 65 |
| IL-12-Fc-686 | p35 D42L | 312 | p40 K260A R261S | 65 |
| IL-12-Fc-687 | p35 D42W | 313 | p40 K260A R261S | 65 |
| IL-12-Fc-688 | p35 E44A | 314 | p40 K260A R261S | 65 |
| IL-12-Fc-689 | p35 E44K | 315 | p40 K260A R261S | 65 |
| IL-12-Fc-690 | p35 E44R | 316 | p40 K260A R261S | 65 |
| IL-12-Fc-691 | p35 D159K | 317 | p40 K260A R261S | 65 |
| IL-12-Fc-692 | p35 D159R | 318 | p40 K260A R261S | 65 |
| IL-12-Fc-693 | p35 D159Y | 319 | p40 K260A R261S | 65 |
| IL-12-Fc-694 | p35 D159F | 320 | p40 K260A R261S | 65 |
| IL-12-Fc-695 | p35 Y161S | 321 | p40 K260A R261S | 65 |
| IL-12-Fc-696 | p35 Y161T | 322 | p40 K260A R261S | 65 |
| IL-12-Fc-697 | p35 Y161A | 323 | p40 K260A R261S | 65 |
| IL-12-Fc-698 | p35 Y161W | 324 | p40 K260A R261S | 65 |
| IL-12-Fc-699 | p35 K162E | 325 | p40 K260A R261S | 65 |
| IL-12-Fc-700 | p35 K162D | 326 | p40 K260A R261S | 65 |

IL-12p35 subunits have N-terminal hexapeptide

TABLE 37

| | p35-Fc (LALADS- | | p40-Fc (LALADS- | |
|---|---|---|---|---|
| | KIH-Knob) | | KIH-Hole) | |
| | IL-12p35 | SEQ ID | IL-12p40 | SEQ ID |
| IL-12-Analog | Subunit | NO: | Subunit | NO: |
| IL-12-Fc-701 | p35 E40K | 304 | p40 K260S R261S | 66 |
| IL-12-Fc-702 | p35 E40R | 305 | p40 K260S R261S | 66 |
| IL-12-Fc-703 | p35 E40W | 306 | p40 K260S R261S | 66 |
| IL-12-Fc-704 | p35 I41A | 307 | p40 K260S R261S | 66 |
| IL-12-Fc-705 | p35 I41V | 308 | p40 K260S R261S | 66 |
| IL-12-Fc-706 | p35 I41S | 309 | p40 K260S R261S | 66 |
| IL-12-Fc-707 | p35 I41T | 310 | p40 K260S R261S | 66 |
| IL-12-Fc-708 | p35 D42A | 311 | p40 K260S R261S | 66 |
| IL-12-Fc-709 | p35 D42L | 312 | p40 K260S R261S | 66 |
| IL-12-Fc-710 | p35 D42W | 313 | p40 K260S R261S | 66 |
| IL-12-Fc-711 | p35 E44A | 314 | p40 K260S R261S | 66 |
| IL-12-Fc-712 | p35 E44K | 315 | p40 K260S R261S | 66 |
| IL-12-Fc-713 | p35 E44R | 316 | p40 K260S R261S | 66 |
| IL-12-Fc-714 | p35 D159K | 317 | p40 K260S R261S | 66 |
| IL-12-Fc-715 | p35 D159R | 318 | p40 K260S R261S | 66 |
| IL-12-Fc-716 | p35 D159Y | 319 | p40 K260S R261S | 66 |
| IL-12-Fc-717 | p35 D159F | 320 | p40 K260S R261S | 66 |
| IL-12-Fc-718 | p35 Y161S | 321 | p40 K260S R261S | 66 |
| IL-12-Fc-719 | p35 Y161T | 322 | p40 K260S R261S | 66 |
| IL-12-Fc-720 | p35 Y161A | 323 | p40 K260S R261S | 66 |
| IL-12-Fc-721 | p35 Y161W | 324 | p40 K260S R261S | 66 |
| IL-12-Fc-722 | p35 K162E | 325 | p40 K260S R261S | 66 |
| IL-12-Fc-723 | p35 K162D | 326 | p40 K260S R261S | 66 |

IL-12p35 subunits have N-terminal hexapeptide

TABLE 38

| | IL-12p35-Fc (LALADS- | | IL-12p40-Fc (LALADS- | |
|---|---|---|---|---|
| | YTE-KIH-Knob) | | YTE-KIH-Hole) | |
| | IL-12p35 | SEQ ID | IL-12p40 | SEQ ID |
| IL-12 Analog | subunit | NO: | subunit | NO: |
| IL-12-Fc-733 | p35 E40K | 347 | WT | 68 |
| IL-12-Fc-734 | p35 E40R | 348 | WT | 68 |
| IL-12-Fc-735 | p35 E40W | 349 | WT | 68 |
| IL-12-Fc-736 | p35 I41A | 350 | WT | 68 |
| IL-12-Fc-737 | p35 I41V | 351 | WT | 68 |
| IL-12-Fc-738 | p35 I41S | 352 | WT | 68 |
| IL-12-Fc-739 | p35 I41T | 353 | WT | 68 |
| IL-12-Fc-740 | p35 D42A | 354 | WT | 68 |
| IL-12-Fc-741 | p35 D42L | 355 | WT | 68 |
| IL-12-Fc-742 | p35 D42W | 356 | WT | 68 |
| IL-12-Fc-743 | p35 E44A | 357 | WT | 68 |
| IL-12-Fc-744 | p35 E44K | 358 | WT | 68 |
| IL-12-Fc-745 | p35 E44R | 359 | WT | 68 |
| IL-12-Fc-746 | p35 D159K | 360 | WT | 68 |
| IL-12-Fc-747 | p35 D159R | 361 | WT | 68 |
| IL-12-Fc-748 | p35 D159Y | 362 | WT | 68 |
| IL-12-Fc-749 | p35 D159F | 363 | WT | 68 |
| IL-12-Fc-750 | p35 Y161S | 364 | WT | 68 |
| IL-12-Fc-751 | p35 Y161T | 365 | WT | 68 |
| IL-12-Fc-752 | p35 Y161A | 366 | WT | 68 |
| IL-12-Fc-753 | p35 Y161W | 367 | WT | 68 |
| IL-12-Fc-754 | p35 K162E | 368 | WT | 68 |
| IL-12-Fc-755 | p35 K162D | 369 | WT | 68 |

WT = wild-type amino acid sequence
IL-12p35 subunits have N-terminal hexapeptide

TABLE 39

| | p35-Fc (LALADS- | | p40-Fc (LALADS- | |
|---|---|---|---|---|
| | YTE-KIH-Knob) | | YTE-KIH-Hole) | |
| | IL-12p35 | SEQ ID | IL-12p40 | SEQ ID |
| IL-12-Analog | Subunit | NO: | Subunit | NO: |
| IL-12-Fc-756 | WT | 390 | p40 K85E | 92 |
| IL-12-Fc-757 | WT | 390 | p40 K85Y | 93 |
| IL-12-Fc-758 | WT | 390 | p40 D87A | 94 |
| IL-12-Fc-759 | WT | 390 | p40 D87L | 95 |
| IL-12-Fc-760 | WT | 390 | p40 I89A | 96 |
| IL-12-Fc-761 | WT | 390 | p40 I89S | 97 |
| IL-12-Fc-762 | WT | 390 | p40 I89D | 98 |
| IL-12-Fc-763 | WT | 390 | p40 I89P | 99 |
| IL-12-Fc-764 | WT | 390 | p40 K104E | 100 |
| IL-12-Fc-765 | WT | 390 | p40 K104D | 101 |
| IL-12-Fc-766 | WT | 390 | p40 K104W | 102 |
| IL-12-Fc-767 | WT | 390 | p40 K104S | 103 |

WT = wild-type amino acid sequence
IL-12p35 subunits have N-terminal hexapeptide

TABLE 40

| | p35-Fc (LALADS- | | p40-Fc (LALADS- | |
|---|---|---|---|---|
| | YTE-KIH-Knob) | | YTE-KIH-Hole) | |
| | IL-12p35 | SEQ ID | IL-12p40 | SEQ ID |
| IL-12-Analog | Subunit | NO: | Subunit | NO: |
| IL-12-Fc-768 | p35 I41S | 352 | p40 D87L | 95 |
| IL-12-Fc-769 | p35 E44R | 359 | p40 D87L | 95 |
| IL-12-Fc-770 | p35 D159K | 360 | p40 D87L | 95 |
| IL-12-Fc-771 | p35 D159R | 361 | p40 D87L | 95 |
| IL-12-Fc-772 | p35 Y161S | 364 | p40 D87L | 95 |
| IL-12-Fc-773 | p35 Y161T | 365 | p40 D87L | 95 |
| IL-12-Fc-774 | p35 Y161A | 366 | p40 D87L | 95 |
| IL-12-Fc-775 | p35 I41S | 352 | p40 K104E | 100 |
| IL-12-Fc-776 | p35 E44R | 359 | p40 K104E | 100 |
| IL-12-Fc-777 | p35 D159K | 360 | p40 K104E | 100 |
| IL-12-Fc-778 | p35 D159R | 361 | p40 K104E | 100 |
| IL-12-Fc-779 | p35 Y161S | 364 | p40 K104E | 100 |
| IL-12-Fc-780 | p35 Y161T | 365 | p40 K104E | 100 |
| IL-12-Fc-781 | p35 Y161A | 366 | p40 K104E | 100 |
| IL-12-Fc-782 | p35 I41S | 352 | p40 K104D | 101 |
| IL-12-Fc-783 | p35 E44R | 359 | p40 K104D | 101 |
| IL-12-Fc-784 | p35 D159K | 360 | p40 K104D | 101 |
| IL-12-Fc-785 | p35 D159R | 361 | p40 K104D | 101 |
| IL-12-Fc-786 | p35 Y161S | 364 | p40 K104D | 101 |
| IL-12-Fc-787 | p35 Y161T | 365 | p40 K104D | 101 |
| IL-12-Fc-788 | p35 Y161A | 366 | p40 K104D | 101 |

IL-12p35 subunits have N-terminal hexapeptide

TABLE 41

| | | | p40-Fc (LALADS-YTE-KIH | |
|---|---|---|---|---|
| | p35-Fc (LALADS-YTE-KIH-Knob) | | Hole) | |
| | | SEQ ID | | SEQ ID |
| IL-12-Analog | IL-12p35 Subunit | NO: | IL-12p40 Subunit | NO: |
| IL-12-Fc-789 | p35 Y161T | 365 | p40 D87L K104E | 124 |
| IL-12-Fc-790 | p35 Y161A | 366 | p40 D87L K104D | 125 |
| IL-12-Fc-791 | p35 D159K Y161T | 370 | p40 D87L K104E | 124 |
| IL-12-Fc-792 | p35 D159K Y161T | 370 | p40 D87L K104D | 125 |
| IL-12-Fc-793 | p35 F33G D159K Y161T | 371 | p40 D87L K104E | 124 |
| IL-12-Fc-794 | p35 F33K D159K Y161T | 372 | p40 D87L K104E | 124 |
| IL-12-Fc-795 | p35 F33S D159K Y161T | 373 | p40 D87L K104E | 124 |
| IL-12-Fc-796 | p35 Y34G D159K Y161T | 374 | p40 D87L K104E | 124 |
| IL-12-Fc-797 | p35 Y34S D159K Y161T | 375 | p40 D87L K104E | 124 |
| IL-12-Fc-798 | p35 Y34T D159K Y161T | 376 | p40 D87L K104E | 124 |
| IL-12-Fc-799 | p35 P35Q D159K Y161T | 377 | p40 D87L K104E | 124 |
| IL-12-Fc-780 | p35 P35E D159K Y161T | 378 | p40 D87L K104E | 124 |
| IL-12-Fc-781 | p35 F33S Y34T D159K Y161T | 379 | p40 D87L K104E | 124 |
| IL-12-Fc-782 | p35 F33S Y34S D159K Y161T | 380 | p40 D87L K104E | 124 |
| IL-12-Fc-783 | p35 F33G Y161T | 381 | p40 D87L K104E | 124 |
| IL-12-Fc-784 | p35 F33K Y161T | 382 | p40 D87L K104E | 124 |
| IL-12-Fc-785 | p35 F33S Y161T | 383 | p40 D87L K104E | 124 |
| IL-12-Fc-786 | p35 Y34G Y161T | 384 | p40 D87L K104E | 124 |
| IL-12-Fc-787 | p35 Y34S Y161T | 385 | p40 D87L K104E | 124 |
| IL-12-Fc-788 | p35 Y34T Y161T | 386 | p40 D87L K104E | 124 |
| IL-12-Fc-789 | p35 F33S Y34T Y161T | 387 | p40 D87L K104E | 124 |
| IL-12-Fc-790 | p35 F33S Y34S Y161T | 388 | p40 D87L K104E | 124 |
| IL-12-Fc-791 | p35 F33S Y34G Y161T | 389 | p40 D87L K104E | 124 |

IL-12p35 subunits have N-terminal hexapeptide

TABLE 42

| | p35-Fc (LALADS-YTE- | | p40-Fc (LALADS-YTE- | |
|---|---|---|---|---|
| | KIH-Knob) | | KIH-Hole) | |
| | | SEQ ID | | SEQ ID |
| IL-12-Analog | IL-12p35 Subunit | NO: | IL-12p40 Subunit | NO: |
| IL-12-Fc-792 | p35 E40K | 347 | p40 143A P215A | 126 |
| IL-12-Fc-793 | p35 E40R | 348 | p40 143A P215A | 126 |
| IL-12-Fc-794 | p35 E40W | 349 | p40 143A P215A | 126 |
| IL-12-Fc-795 | p35 I41A | 350 | p40 143A P215A | 126 |
| IL-12-Fc-796 | p35 I41V | 351 | p40 143A P215A | 126 |
| IL-12-Fc-797 | p35 I41S | 352 | p40 143A P215A | 126 |
| IL-12-Fc-798 | p35 I41T | 353 | p40 143A P215A | 126 |
| IL-12-Fc-799 | p35 D42A | 354 | p40 143A P215A | 126 |
| IL-12-Fc-800 | p35 D42L | 355 | p40 143A P215A | 126 |
| IL-12-Fc-801 | p35 D42W | 356 | p40 143A P215A | 126 |
| IL-12-Fc-802 | p35 E44A | 357 | p40 143A P215A | 126 |
| IL-12-Fc-803 | p35 E44K | 358 | p40 143A P215A | 126 |
| IL-12-Fc-804 | p35 E44R | 359 | p40 143A P215A | 126 |
| IL-12-Fc-805 | p35 D159K | 360 | p40 143A P215A | 126 |
| IL-12-Fc-806 | p35 D159R | 361 | p40 143A P215A | 126 |
| IL-12-Fc-807 | p35 D159Y | 362 | p40 143A P215A | 126 |
| IL-12-Fc-808 | p35 D159F | 363 | p40 143A P215A | 126 |
| IL-12-Fc-809 | p35 Y161S | 364 | p40 143A P215A | 126 |
| IL-12-Fc-810 | p35 Y161T | 365 | p40 143A P215A | 126 |
| IL-12-Fc-811 | p35 Y161A | 366 | p40 143A P215A | 126 |
| IL-12-Fc-812 | p35 Y161W | 367 | p40 143A P215A | 126 |
| IL-12-Fc-813 | p35 K162E | 368 | p40 143A P215A | 126 |
| IL-12-Fc-814 | p35 K162D | 369 | p40 143A P215A | 126 |

IL-12p35 subunits have N-terminal hexapeptide

TABLE 43

| | p35-Fc (LALADS-YTE-KIH- | | | |
|---|---|---|---|---|
| | Knob) | | p40-Fc (LALADS-YTE-KIH-Hole) | |
| | | SEQ ID | | SEQ ID |
| IL-12-Analog | IL-12p35 Subunit | NO: | IL-12p40 Subunit | NO: |
| IL-12-Fc-815 | p35 E40K | 347 | p40 P143A P215A K260A R261S V303I | 127 |
| IL-12-Fc-816 | p35 E40R | 348 | p40 P143A P215A K260A R261S V303I | 127 |
| IL-12-Fc-817 | p35 E40W | 349 | p40 P143A P215A K260A R261S V303I | 127 |
| IL-12-Fc-818 | p35 I41A | 350 | p40 P143A P215A K260A R261S V303I | 127 |
| IL-12-Fc-819 | p35 I41V | 351 | p40 P143A P215A K260A R261S V303I | 127 |
| IL-12-Fc-820 | p35 I41S | 352 | p40 P143A P215A K260A R261S V303I | 127 |
| IL-12-Fc-821 | p35 I41T | 353 | p40 P143A P215A K260A R261S V303I | 127 |
| IL-12-Fc-822 | p35 D42A | 354 | p40 P143A P215A K260A R261S V303I | 127 |
| IL-12-Fc-823 | p35 D42L | 355 | p40 P143A P215A K260A R261S V303I | 127 |
| IL-12-Fc-824 | p35 D42W | 356 | p40 P143A P215A K260A R261S V303I | 127 |
| IL-12-Fc-825 | p35 E44A | 357 | p40 P143A P215A K260A R261S V303I | 127 |
| IL-12-Fc-826 | p35 E44K | 358 | p40 P143A P215A K260A R261S V303I | 127 |
| IL-12-Fc-827 | p35 E44R | 359 | p40 P143A P215A K260A R261S V303I | 127 |
| IL-12-Fc-828 | p35 D159K | 360 | p40 P143A P215A K260A R261S V303I | 127 |
| IL-12-Fc-829 | p35 D159R | 361 | p40 P143A P215A K260A R261S V303I | 127 |
| IL-12-Fc-830 | p35 D159Y | 362 | p40 P143A P215A K260A R261S V303I | 127 |
| IL-12-Fc-831 | p35 D159F | 363 | p40 P143A P215A K260A R261S V303I | 127 |
| IL-12-Fc-832 | p35 Y161S | 364 | p40 P143A P215A K260A R261S V303I | 127 |
| IL-12-Fc-833 | p35 Y161T | 365 | p40 P143A P215A K260A R261S V303I | 127 |
| IL-12-Fc-834 | p35 Y161A | 366 | p40 P143A P215A K260A R261S V303I | 127 |
| IL-12-Fc-835 | p35 Y161W | 367 | p40 P143A P215A K260A R261S V303I | 127 |
| IL-12-Fc-836 | p35 K162E | 368 | p40 P143A P215A K260A R261S V303I | 127 |
| IL-12-Fc-837 | p35 K162D | 369 | p40 P143A P215A K260A R261S V303I | 127 |

IL-12p35 subunits have N-terminal hexapeptide

TABLE 44

| | p35-Fc (LALADS-YTE-KIH-Knob) | | | p40-Fc (LALADS-YTE-KIH-Hole) | |
|---|---|---|---|---|---|
| IL-12- | | | SEQ | | SEQ |
| Analog | IL-12p35 Subunit | | ID NO: | IL-12p40 Subunit | ID NO: |
| IL-12-Fc-838 | p35 | E40K | 347 | p40 P143A P215A K260S R261S V303I | 128 |
| IL-12-Fc-839 | p35 | E40R | 348 | p40 P143A P215A K260S R261S V303I | 128 |
| IL-12-Fc-840 | p35 | E40W | 349 | p40 P143A P215A K260S R261S V303I | 128 |
| IL-12-Fc-841 | p35 | I41A | 350 | p40 P143A P215A K260S R261S V303I | 128 |
| IL-12-Fc-842 | p35 | I41V | 351 | p40 P143A P215A K260S R261S V303I | 128 |
| IL-12-Fc-843 | p35 | I41S | 352 | p40 P143A P215A K260S R261S V303I | 128 |
| IL-12-Fc-844 | p35 | I41T | 353 | p40 P143A P215A K260S R261S V303I | 128 |
| IL-12-Fc-845 | p35 | D42A | 354 | p40 P143A P215A K260S R261S V303I | 128 |
| IL-12-Fc-846 | p35 | D42L | 355 | p40 P143A P215A K260S R261S V303I | 128 |
| IL-12-Fc-847 | p35 | D42W | 356 | p40 P143A P215A K260S R261S V303I | 128 |
| IL-12-Fc-848 | p35 | E44A | 357 | p40 P143A P215A K260S R261S V303I | 128 |
| IL-12-Fc-849 | p35 | E44K | 358 | p40 P143A P215A K260S R261S V303I | 128 |
| IL-12-Fc-850 | p35 | E44R | 359 | p40 P143A P215A K260S R261S V303I | 128 |
| IL-12-Fc-851 | p35 | D159K | 360 | p40 P143A P215A K260S R261S V303I | 128 |
| IL-12-Fc-852 | p35 | D159R | 361 | p40 P143A P215A K260S R261S V303I | 128 |
| IL-12-Fc-853 | p35 | D159Y | 362 | p40 P143A P215A K260S R261S V303I | 128 |
| IL-12-Fc-854 | p35 | D159F | 363 | p40 P143A P215A K260S R261S V303I | 128 |
| IL-12-Fc-855 | p35 | Y161S | 364 | p40 P143A P215A K260S R261S V303I | 128 |
| IL-12-Fc-856 | p35 | Y161T | 365 | p40 P143A P215A K260S R261S V303I | 128 |
| IL-12-Fc-857 | p35 | Y161A | 366 | p40 P143A P215A K260S R261S V303I | 128 |
| IL-12-Fc-858 | p35 | Y161W | 367 | p40 P143A P215A K260S R261S V303I | 128 |
| IL-12-Fc-859 | p35 | K162E | 368 | p40 P143A P215A K260S R261S V303I | 128 |
| IL-12-Fc-860 | p35 | K162D | 369 | p40 P143A P215A K260S R261S V303I | 128 |

IL-12p35 subunits have N-terminal hexapeptide

TABLE 45

| | p35-Fc (LALADS-YTE-KIH-Knob) | | | p40-Fc (LALADS-YTE-KIH-Hole) | |
|---|---|---|---|---|---|
| IL-12- | | | SEQ | | SEQ |
| Analog | IL-12p35 Subunit | | ID NO: | IL-12p40 Subunit | ID NO: |
| IL-12-Fc-861 | p35 | E40K | 347 | p40 K260A R261S V303I | 129 |
| IL-12-Fc-862 | p35 | E40R | 348 | p40 K260A R261S V303I | 129 |
| IL-12-Fc-863 | p35 | E40W | 349 | p40 K260A R261S V303I | 129 |
| IL-12-Fc-864 | p35 | I41A | 350 | p40 K260A R261S V303I | 129 |
| IL-12-Fc-865 | p35 | I41V | 351 | p40 K260A R261S V303I | 129 |
| IL-12-Fc-866 | p35 | I41S | 352 | p40 K260A R261S V303I | 129 |
| IL-12-Fc-867 | p35 | I41T | 353 | p40 K260A R261S V303I | 129 |
| IL-12-Fc-868 | p35 | D42A | 354 | p40 K260A R261S V303I | 129 |
| IL-12-Fc-869 | p35 | D42L | 355 | p40 K260A R261S V303I | 129 |
| IL-12-Fc-870 | p35 | D42W | 356 | p40 K260A R261S V303I | 129 |
| IL-12-Fc-870 | p35 | E44A | 357 | p40 K260A R261S V303I | 129 |
| IL-12-Fc-871 | p35 | E44K | 358 | p40 K260A R261S V303I | 129 |
| IL-12-Fc-872 | p35 | E44R | 359 | p40 K260A R261S V303I | 129 |
| IL-12-Fc-873 | p35 | D159K | 360 | p40 K260A R261S V303I | 129 |
| IL-12-Fc-874 | p35 | D159R | 361 | p40 K260A R261S V303I | 129 |
| IL-12-Fc-875 | p35 | D159Y | 362 | p40 K260A R261S V303I | 129 |
| IL-12-Fc-876 | p35 | D159F | 363 | p40 K260A R261S V303I | 129 |
| IL-12-Fc-877 | p35 | Y161S | 364 | p40 K260A R261S V303I | 129 |
| IL-12-Fc-878 | p35 | Y161T | 365 | p40 K260A R261S V303I | 129 |
| IL-12-Fc-879 | p35 | Y161A | 366 | p40 K260A R261S V303I | 129 |
| IL-12-Fc-880 | p35 | Y161W | 367 | p40 K260A R261S V303I | 129 |
| IL-12-Fc-881 | p35 | K162E | 368 | p40 K260A R261S V303I | 129 |
| IL-12-Fc-882 | p35 | K162D | 369 | p40 K260A R261S V303I | 129 |

IL-12p35 subunits have N-terminal hexapeptide

TABLE 46

| | p35-Fc (LALADS-YTE-KIH-Knob) | | | p40-Fc (LALADS-YTE-KIH-Hole) | |
|---|---|---|---|---|---|
| | | | SEQ | | SEQ |
| IL-12-Analog | IL-12p35 Subunit | | ID NO: | IL-12p40 Subunit | ID NO: |
| IL-12-Fc-883 | p35 | E40K | 347 | p40 K260S R261S V303I | 130 |
| IL-12-Fc-884 | p35 | E40R | 348 | p40 K260S R261S V303I | 130 |
| IL-12-Fc-885 | p35 | E40W | 349 | p40 K260S R261S V303I | 130 |
| IL-12-Fc-886 | p35 | I41A | 350 | p40 K260S R261S V303I | 130 |
| IL-12-Fc-887 | p35 | I41V | 351 | p40 K260S R261S V303I | 130 |
| IL-12-Fc-888 | p35 | I41S | 352 | p40 K260S R261S V303I | 130 |
| IL-12-Fc-889 | p35 | I41T | 353 | p40 K260S R261S V303I | 130 |
| IL-12-Fc-890 | p35 | D42A | 354 | p40 K260S R261S V303I | 130 |
| IL-12-Fc-891 | p35 | D42L | 355 | p40 K260S R261S V303I | 130 |
| IL-12-Fc-892 | p35 | D42W | 356 | p40 K260S R261S V303I | 130 |
| IL-12-Fc-893 | p35 | E44A | 357 | p40 K260S R261S V303I | 130 |
| IL-12-Fc-894 | p35 | E44K | 358 | p40 K260S R261S V303I | 130 |
| IL-12-Fc-895 | p35 | E44R | 359 | p40 K260S R261S V303I | 130 |
| IL-12-Fc-896 | p35 | D159K | 360 | p40 K260S R261S V303I | 130 |
| IL-12-Fc-897 | p35 | D159R | 361 | p40 K260S R261S V303I | 130 |
| IL-12-Fc-898 | p35 | D159Y | 362 | p40 K260S R261S V303I | 130 |
| IL-12-Fc-899 | p35 | D159F | 363 | p40 K260S R261S V303I | 130 |
| IL-12-Fc-900 | p35 | Y161S | 364 | p40 K260S R261S V303I | 130 |
| IL-12-Fc-901 | p35 | Y161T | 365 | p40 K260S R261S V303I | 130 |
| IL-12-Fc-902 | p35 | Y161A | 366 | p40 K260S R261S V303I | 130 |
| IL-12-Fc-903 | p35 | Y161W | 367 | p40 K260S R261S V303I | 130 |
| IL-12-Fc-904 | p35 | K162E | 368 | p40 K260S R261S V303I | 130 |
| IL-12-Fc-905 | p35 | K162D | 369 | p40 K260S R261S V303I | 130 |

IL-12p35 subunits have N-terminal hexapeptide

TABLE 47

| | p35-Fc (LALADS-YTE-KIH-Knob) | | | p40-Fc (LALADS-YTE-KIH-Hole) | |
|---|---|---|---|---|---|
| | | | SEQ | | SEQ |
| IL-12-Analog | IL-12p35 Subunit | | ID NO: | IL-12p40 Subunit | ID NO: |
| IL-12-Fc-906 | p35 | E40K | 347 | p40 K260A R261S | 131 |
| IL-12-Fc-907 | p35 | E40R | 348 | p40 K260A R261S | 131 |
| IL-12-Fc-908 | p35 | E40W | 349 | p40 K260A R261S | 131 |
| IL-12-Fc-909 | p35 | I41A | 350 | p40 K260A R261S | 131 |
| IL-12-Fc-910 | p35 | I41V | 351 | p40 K260A R261S | 131 |
| IL-12-Fc-911 | p35 | I41S | 352 | p40 K260A R261S | 131 |
| IL-12-Fc-912 | p35 | I41T | 353 | p40 K260A R261S | 131 |
| IL-12-Fc-913 | p35 | D42A | 354 | p40 K260A R261S | 131 |
| IL-12-Fc-914 | p35 | 5D42L | 355 | p40 K260A R261S | 131 |
| IL-12-Fc-915 | p35 | D42W | 356 | p40 K260A R261S | 131 |
| IL-12-Fc-916 | p35 | E44A | 357 | p40 K260A R261S | 131 |
| IL-12-Fc-917 | p35 | E44K | 358 | p40 K260A R261S | 131 |
| IL-12-Fc-918 | p35 | E44R | 359 | p40 K260A R261S | 131 |
| IL-12-Fc-919 | p35 | D159K | 360 | p40 K260A R261S | 131 |
| IL-12-Fc-920 | p35 | D159R | 361 | p40 K260A R261S | 131 |
| IL-12-Fc-921 | p35 | D159Y | 362 | p40 K260A R261S | 131 |
| IL-12-Fc-922 | p35 | D159F | 363 | p40 K260A R261S | 131 |
| IL-12-Fc-923 | p35 | Y161S | 364 | p40 K260A R261S | 131 |
| IL-12-Fc-924 | p35 | Y161T | 365 | p40 K260A R261S | 131 |
| IL-12-Fc-925 | p35 | Y161A | 366 | p40 K260A R261S | 131 |
| IL-12-Fc-926 | p35 | Y161W | 367 | p40 K260A R261S | 131 |
| IL-12-Fc-927 | p35 | K162E | 368 | p40 K260A R261S | 131 |
| IL-12-Fc-928 | p35 | K162D | 369 | p40 K260A R261S | 131 |

IL-12p35 subunits have N-terminal hexapeptide

TABLE 48

| | p35-Fc (LALADS-YTE-KIH-Knob) | | | p40-Fc (LALADS-YTE-KIH-Hole) | |
|---|---|---|---|---|---|
| | | | SEQ | | SEQ |
| IL-12-Analog | IL-12p35 Subunit | | ID NO: | IL-12p40 Subunit | ID NO: |
| IL-12-Fc-929 | p35 | E40K | 347 | p40 K260S R261S | 132 |
| IL-12-Fc-930 | p35 | E40R | 348 | p40 K260S R261S | 132 |
| IL-12-Fc-931 | p35 | E40W | 349 | p40 K260S R261S | 132 |
| IL-12-Fc-932 | p35 | I41A | 350 | p40 K260S R261S | 132 |
| IL-12-Fc-933 | p35 | I41V | 351 | p40 K260S R261S | 132 |
| IL-12-Fc-934 | p35 | I41S | 352 | p40 K260S R261S | 132 |
| IL-12-Fc-935 | p35 | I41T | 353 | p40 K260S R261S | 132 |
| IL-12-Fc-936 | p35 | D42A | 354 | p40 K260S R261S | 132 |
| IL-12-Fc-937 | p35 | D42L | 355 | p40 K260S R261S | 132 |
| IL-12-Fc-938 | p35 | D42W | 356 | p40 K260S R261S | 132 |
| IL-12-Fc-930 | p35 | E44A | 357 | p40 K260S R261S | 132 |
| IL-12-Fc-940 | p35 | E44K | 358 | p40 K260S R261S | 132 |
| IL-12-Fc-941 | p35 | E44R | 359 | p40 K260S R261S | 132 |
| IL-12-Fc-942 | p35 | D159K | 360 | p40 K260S R261S | 132 |
| IL-12-Fc-943 | p35 | D159R | 361 | p40 K260S R261S | 132 |
| IL-12-Fc-944 | p35 | D159Y | 362 | p40 K260S R261S | 132 |
| IL-12-Fc-945 | p35 | D159F | 363 | p40 K260S R261S | 132 |
| IL-12-Fc-946 | p35 | Y161S | 364 | p40 K260S R261S | 132 |
| IL-12-Fc-947 | p35 | Y161T | 365 | p40 K260S R261S | 132 |
| IL-12-Fc-948 | p35 | Y161A | 366 | p40 K260S R261S | 132 |
| IL-12-Fc-949 | p35 | Y161W | 367 | p40 K260S R261S | 132 |
| IL-12-Fc-950 | p35 | K162E | 368 | p40 K260S R261S | 132 |
| IL-12-Fc-951 | p35 | K162D | 369 | p40 K260S R261S | 132 |

IL-12p35 subunits have N-terminal hexapeptide

The IL-12-Fc analogs shown in Tables 27-48 comprise the IL-12p35 subunit N-terminal hexapeptide having the amino acid sequence RNLPVA (SEQ ID NO: 238), which is present in mature wild-type IL-12p35. However, in particular embodiments, the IL-12p35 subunit of the IL-12-Fc analogs shown in Tables 27-48 comprises a deletion of the N-terminal R residue, a deletion of the N-terminal RN dipeptide, a deletion of the N-terminal RNL tripeptide, a deletion of the N-terminal RNLP tetrapeptide (SEQ ID NO: 390), or a deletion of the N-terminal RNLPV pentapeptide (SEQ ID NO: 391). In further embodiments, the ILp35 subunit shown in Tables 27-48 may comprise a deletion of the N-terminal septapeptide RNLPVAT (SEQ ID NO: 392), a deletion of the N-terminal octapeptide RNLPVATP (SEQ ID NO: 393), a deletion of the N-terminal nonapeptide RNLPVATPD (SEQ ID NO: 394), or a deletion of the N-terminal decapeptide RNLPVATPDP (SEQ ID NO: 395).

### IV. Nucleic Acid Molecules Encoding the Monovalent IL-12 Heterodimeric Fc Proteins of the Present Invention

The present invention further provides nucleic acid molecules that encode the monovalent IL-12 heterodimeric Fc proteins of the present invention. In particular embodiments, the p35 subunit fused the N-terminus of a first Fc fragment (p35-Fc) is encoded by a first nucleic acid molecule, and the p40 subunit fused to the N-terminus of a second Fc fragment (p40-Fc) is encoded by a second nucleic acid molecule, wherein the first Fc and the second Fc comprise mutations such that only Fc heterodimers, i.e., first Fc:second Fc heterodimer pair.

In particular embodiments, the p35-Fc and p40-Fc are expressed as a fusion protein in which the N-terminus of the p35-Fc and p40-Fc are each fused at the N-terminus to a leader peptide to facilitate the transport of the respective p35-Fc and p40-Fc through the secretory pathway. Examples of leader/signal peptides that may be used include those comprising the amino acid sequence set forth in SEQ ID NO: 199, SEQ ID NO: 200, or SEQ ID NO: 201.

The nucleic acid molecules disclosed herein may include one or more substitutions that optimize one or more of the codons for enhancing the expression of the nucleic acid molecule in a particular host cell, e.g., yeast or fungal host cell, non-human mammalian host cell, human host cell, insect host cell, or prokaryote host cell.

Exemplary nucleotide sequences encoding: IL-12p35 (I41S) linked via 15GS linker to Fc-LALADS-KIH Knob: S354C-T366W is set forth in SEQ ID NO: 223, IL-12p35 (E44R) linked via 15GS linker to Fc-LALADS-KIH Knob: S354C-T366W is set forth in SEQ ID NO: 224, IL-12p35 (D159K) linked via 15GS linker to Fc-LALADS-KIH Knob: S354C-T366W is set forth in SEQ ID NO: 225, IL-12p35 (D159R) linked via 15GS linker to Fc-LALADS-KIH Knob: S354C-T366W is set forth in SEQ ID NO: 226, IL-12p35 (Y161S) linked via 15GS linker to Fc-LALADS-KIH Knob: S354C-T366W is set forth in SEQ ID NO: 227, IL-12p35 (Y161T) linked via 15GS linker to Fc-LALADS-KIH Knob: S354C-T366W is set forth in SEQ ID NO: 228, IL-12p35 (Y161A) linked via 15GS linker to Fc-LALADS-KIH Knob: S354C-T366W is set forth in SEQ ID NO: 229, IL-12p35 (Δhexapeptide) linked via 15GS linker to Fc-LALADS-KIH Knob: S354C-T366W is set forth in SEQ ID NO: 249, and IL-12p35 (+hexapeptide) linked via 15GS linker to Fc-LALADS-KIH Knob: S354C-T366W is set forth in SEQ ID NO: 250.

Exemplary nucleotide sequence encoding IL-12p40 linked via 4G linker to Fc-LALADS-KIH Hole: Y349C-T366S-L368A-Y407V is set forth in SEQ ID NO: 230. Exemplary nucleotide sequence encoding IL-12p40 linked via 4G linker to Fc-LALADS-KIH Hole: Y349C-T366S-L368A-Y407V is set forth in SEQ ID NO: 230, IL-12p40 (D87L) linked via 4G linker to Fc-LALADS-KIH Hole: Y349C-T366S-L368A-Y407V is set forth in SEQ ID NO: 231, IL-12p40 (K104E) linked via 4G linker to Fc-LALADS-KIH Hole: Y349C-T366S-L368A-Y407V is set forth in SEQ ID NO: 232, and IL-12p40 (K104E) linked via 4G linker to Fc-LALADS-KIH Hole: Y349C-T366S-L368A-Y407V is set forth in SEQ ID NO: 230.

Exemplary nucleotide sequence encoding IL-12p35 (I41S) linked via 15GS linker to Fc-LALADS-YTE-KIH Knob: S354C-T366W is set forth in SEQ ID NO: 240, IL-12p35 (E44R) linked via 15GS linker to Fc-LALADS-YTE-KIH Knob: S354C-T366W is set forth in SEQ ID NO: 241, IL-12p35 (D159K) linked via 15GS linker to Fc-LALADS-YTE-KIH Knob: S354C-T366W is set forth in SEQ ID NO: 242, IL-12p35 (D159R) linked via 15GS linker to Fc-LALADS-YTE-KIH Knob: S354C-T366W is set forth in SEQ ID NO: 243, IL-12p35 (Y161A) linked via 15GS linker to Fc-LALADS-YTE-KIH Knob: S354C-T366W is set forth in SEQ ID NO: 244, IL-12p35 (Y161S) linked via 15GS linker to Fc-LALADS-YTE-KIH Knob: S354C-T366W is set forth in SEQ ID NO: 234, IL-12p35 (Y161T) linked via 15GS linker to Fc-LALADS-YTE-KIH Knob: S354C-T366W is set forth in SEQ ID NO: 235, IL-12p35 (+hexapeptide) linked via 15GS linker to Fc-LALADS-YTE-KIH Knob: S354C-T366W is set forth in SEQ ID NO: 236, and IL-12p35 (Δhexapeptide) linked via 15GS linker to Fc-LALADS-YTE-KIH Knob: S354C-T366W is set forth in SEQ ID NO: 248.

Exemplary nucleotide sequence encoding IL-12p40 linked via 4G linker to Fc-LALADS-YTE KIH Hole: Y349C-T366S-L368A-Y407V is set forth in SEQ ID NO: 237, IL-12p40 (D87L) linked via 4G linker to Fc-LALADS-YTE KIH Hole: Y349C-T366S-L368A-Y407V is set forth in SEQ ID NO: 245, IL-12p40 (K104E) linked via 4G linker to Fc-LALADS-YTE KIH Hole: Y349C-T366S-L368A-Y407V is set forth in SEQ ID NO: 246, and IL-12p40 (K104D) linked via 4G linker to Fc-LALADS-YTE KIH Hole: Y349C-T366S-L368A-Y407V is set forth in SEQ ID NO: 247.

### V. Methods for Making Monovalent IL-12 Heterodimeric Fc Proteins of the Present Invention

The present invention includes recombinant methods for making an IL-12 heterodimeric Fc protein of the present invention comprising introducing into a host cell (i) an expression vector comprising nucleic acid molecule(s) that encode the IL-12p35-Fc or the IL-12p40-Fc, or (ii) two expression vectors comprising nucleic acid molecules, one vector comprising a nucleic acid molecule encoding the IL-12p35-Fc, the other vector comprising a nucleic acid molecule encoding the IL-12p40-Fc. The nucleic acid molecules or polynucleotides encoding the IL-12p35-Fc and the IL-12p40-Fc are each operably linked to a promoter and other transcription and translation regulatory sequences. The host cell is cultured under conditions and a time period suitable for expression of the IL-12p35-Fc and the IL-12p40-Fc from the nucleic acid molecules followed by isolating the IL-12 heterodimeric Fc protein from the host cell and/or medium in which the host cell is grown. See e.g., WO2004041862, WO2006122786, WO2008020079, WO2008142164 or WO2009068627. The expression vector may be a plasmid or viral vector. The invention also relates to host cells that comprise such nucleic acid molecules encoding the monovalent IL-12 heterodimeric Fc proteins (host cells comprising nucleic acid molecules encoding the IL-12p35-Fc and IL-12p40-Fc).

Eukaryotic and prokaryotic host cells, including mammalian cells, as hosts for expression of proteins are well known in the art and include many immortalized cell lines available from the American Type Culture Collection (ATCC). These include, but are not limited to, Chinese hamster ovary (CHO) cells, NSO cells, SP2 cells, HeLa cells, baby hamster kidney (BHK) cells, monkey kidney cells (COS), human hepatocellular carcinoma cells (e.g., Hep G2), A549 cells, 3T3 cells, human embryonic kidney (HEK)-293 cells and a number of other cell lines. Thus, mammalian host cells include human, mouse, rat, dog, monkey, pig, goat, bovine, horse, and hamster cells. In particular, cell lines are selected through determining which cell lines have high expression levels. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia pastoris, Saccharomyces cerevisiae, and Trichoderma reesei. The present invention further includes any host cell comprising an IL-12 heterodimeric Fc protein of the present invention or comprising one or more nucleic acid molecules encoding such IL-12 heterodimeric Fc protein or comprising an expression vector that comprises one or more nucleic acid molecules encoding such IL-12 heterodimeric Fc protein.

Further, expression of an IL-12 heterodimeric Fc protein from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0216846B1, 0256055B1, 0323997B1, and 0338841B1. Thus, in an embodiment of the invention, the mammalian host cells lack a glutamine synthetase gene and are grown in the absence of glutamine in the medium wherein, however, the nucleic acid molecule encoding the immunoglobulin chain comprises a glutamine synthetase gene which complements the lack of the gene in the host cell. Such host cells containing the IL-12 heterodimeric Fc protein or nucleic acid molecule(s) or expression vector(s) as discussed herein as well as expression methods, as discussed herein, for making the IL-12 heterodimeric Fc protein using such a host cell are part of the present invention.

The present invention further includes methods for purifying an IL-12 heterodimeric Fc protein comprising introducing a sample (e.g., culture medium, cell lysate or cell lysate fraction, e.g., a soluble fraction of the lysate) comprising the IL-12 heterodimeric Fc protein to a purification medium (e.g., cation-exchange medium, anion-exchange medium and/or hydrophobic exchange medium) and either collecting purified IL-12 heterodimeric Fc protein from the flow-through fraction of said sample that does not bind to the medium; or, discarding the flow-through fraction and eluting bound IL-12 heterodimeric Fc protein from the medium and collecting the eluate. In an embodiment of the invention, the medium is in a column to which the sample is applied. In an embodiment of the invention, the purification method is conducted following recombinant expression of the IL-12 heterodimeric Fc protein in a host cell, e.g., wherein the host cell is first lysed and, optionally, the lysate is purified of insoluble materials prior to purification on a medium; or wherein the IL-12 heterodimeric Fc protein is secreted into the culture medium by the host cell and the medium or a fraction thereof is applied to the purification medium.

### VI. N-Glycosylated Monovalent IL-12 Heterodimeric Fc Proteins of the Present Invention

IL-12 is a glycoprotein comprising three N-glycosylation sites on the IL-12p35 subunit and four N-glycosylation sites on the IL-12p40 subunit. On the mature Δhexapeptide IL-12p35 subunit, the N-glycosylation sites are at amino acid position N65 of the amino acid sequence NES, N79 of the amino acid sequence NGS, and N189 of the amino acid sequence NAS as represented by the amino acid sequence set forth in SEQ ID NO: 1 for the mature Δhexapeptide IL-12p35 Fc protein. On the mature+hexapeptide IL-12p35 subunit, the N-glycosylation sites are at amino acid position N71 of the amino acid sequence NES, N85 of the amino acid sequence NGS, and N289 of the amino acid sequence NAS. On the mature IL-12p40 subunit, the N-glycosylation sites are at amino acid position N103 of the amino acid sequence NKT, N113 of the amino acid sequence NYS, N200 of the amino acid sequence NYT, and N281 of the amino acid sequence NAS as represented by the amino acid sequence set forth in SEQ ID NO: 2 for the mature IL-12p40 Fc protein. The N-glycosylation site on the Fc portion of the protein is at position N297 of the amino acid sequence NST wherein the numbering is according to the Eu numbering scheme. Eu position N297 corresponds to amino acid position 283 of the amino acid sequence shown in SEQ ID NO: 1 for the mature Δhexapeptide IL-12p35 Fc protein and to amino acid position 387 of the amino acid sequence shown in SEQ ID NO: 2 for the mature IL-12p40 Fc protein. For the mature+hexapeptide IL-12p35 Fc protein, Eu position N297 corresponds to amino acid position 289.

In general, glycoproteins produced in a particular cell line or transgenic animal will have a glycosylation pattern that is characteristic for glycoproteins produced in the cell line or transgenic animal. Therefore, the particular glycosylation pattern of an IL-12 heterodimeric Fc protein will depend on the particular cell line or transgenic animal used to produce the IL-12 heterodimeric Fc protein. Monovalent IL-12 heterodimeric Fc proteins comprising N-linked glycans may be produced in mammalian or human cells (mammalian/human N-linked glycans, e.g., Chinese hamster ovary (CHO) cells (CHO N-linked glycans), or in yeast cells genetically engineered to produce mammalian or human-like N-glycans (engineered yeast N-linked glycans), such as, for example, Pichia pastoris genetically engineered to produce mammalian or human-like N-glycans (See Nett et al. Yeast 28: 237-252 (2011); Hamilton et al. Science. 313: 1441-1443 (2006); Hamilton et al. Curr. Opin. Biotechnol. 18(5): 387-392 (2007)). Compositions of glycoproteins produced in mammalian or human cells will comprise a heterogenous mixture of N-glycans with particular N-glycan species being more predominant and N-glycan occupancy at particular N-glycosylation sites higher than at other N-glycosylation sites. The types of N-glycans, the particular predominant N-glycan species, and the percent occupancy of particular N-glycosylation sites is specific for the particular glycoprotein being produced.

The applicants have found that while the sialylation of the N-glycans of IL-12 heterodimeric Fc proteins disclosed herein does not appear to impact potency in activated rhesus PBMC and HEK cell line, compositions of IL-12 heterodimeric Fc proteins disclosed herein in which sialylation of the N-glycans is between 15% and 25% appears to enhance IL-12 heterodimeric Fc protein pharmacokinetics in TG32 Mouse PK studies (See Roopenian et al., Methods Mol. Biol. 602:93-104 (2010) for discussion of TG32 mice.) Methods for producing glycoproteins in mammalian or human cells with various yields of sialylated N-glycans are known in the art. See for example, Weikert et al., Nat. Biotech. 17: 1116-1121 (1999); Nam et al., Biotechnol. Bioeng. 100: 1178-1192 (2008); Bork et al., J. Pharm. Sci. 98: 3499-3508 (2009); Guh et al., Bioengineered 5: 269-273 (2014); and Kwak et al., Sci. Reports &: article No. 13059 (2017).

In an embodiment of the invention, the IL-12 heterodimeric Fc protein comprises one or more fucosylated and/or non-fucosylated GlcNAc-terminated N-glycans, galactose-terminated N-glycans, or sialylated N-glycans, and mixtures thereof. In an embodiment of the invention, compositions of monovalent IL-12 heterodimeric Fc proteins are provided in which at least 15% of the total N-glycans therein are sialylated. In an embodiment of the invention, compositions of monovalent IL-12 heterodimeric Fc proteins are provided in which 15% to 25% of the total N-glycans therein are sialylated. In an embodiment of the invention, compositions of monovalent IL-12 heterodimeric Fc proteins are provided in which in which at least 20% of the total N-glycans therein are sialylated. In an embodiment of the invention, compositions of monovalent IL-12 heterodimeric Fc proteins are provided in which in which at least 25% of the total N-glycans therein are sialylated.

In particular embodiments of compositions comprising the Δhexapeptide IL-12p35 Fc protein produced in CHO cells, the predominant N-glycan at amino acid position N65 and N79 are N-glycans A3G3F1S1 and A3G3F1S2; the predominant N-glycan at amino acid position N189 is Man6; and the predominant N-glycans at position N189 are G0F and G1F. In particular embodiments of compositions comprising the IL-12p40 Fc protein produced in CHO cells, the predominant N-glycans at amino acid position N103 are N-glycans G2FS and G2F; the predominant N-glycan at amino acid position N200 is Man9; the predominant N-glycans at position N281 are G2FS and G2FS2; and, the predominant N-glycans at position N189 are G0F and G1F.

In general, glycoproteins produced in a particular cell line or transgenic animal will have a glycosylation pattern that is characteristic for glycoproteins produced in the cell line or transgenic animal. Therefore, the particular glycosylation pattern of an IL-12 heterodimeric Fc protein will depend on the particular cell line or transgenic animal used to produce the IL-12 heterodimeric Fc protein. Monovalent IL-12 heterodimeric Fc proteins comprising only non-fucosylated N-glycans are part of the present invention and may be advantageous, because non-fucosylated antibodies have been shown to typically exhibit more potent efficacy than their fucosylated counterparts both in vitro and in vivo (See for example, Shinkawa et al., J. Biol. Chem. 278: 3466-3473 (2003); U.S. Pat. Nos. 6,946,292 and 7,214,775). These monovalent IL-12 heterodimeric Fc proteins with non-fucosylated N-glycans are not likely to be immunogenic because their carbohydrate structures are a normal component of the population that exists in human serum IgG.

The present invention includes monovalent IL-12 heterodimeric Fc proteins comprising N-linked glycans that are typically added to immunoglobulins produced in Chinese hamster ovary cells (“CHO N-linked glycans”) or to immunoglobulins produced in yeast cells such as, for example, Pichia pastoris, which have been engineered to produce glycoproteins comprising human-like or mammalian-like N-glycans (“engineered yeast N-linked glycans”).

For example, in an embodiment of the invention, the IL-12 heterodimeric Fc protein comprises one or more “engineered yeast N-linked glycans” or “CHO N-linked glycans” in which 15% to 25% thereof are sialylated. See Nett et al. Yeast 28: 237-252 (2011); Hamilton et al. Science. 313: 1441-1443 (2006); Hamilton et al. Curr. Opin. Biotechnol. 18(5): 387-392 (2007) for methods for making engineered yeast cells that make sialylated N-glycans. For example, in an embodiment of the invention, an engineered yeast cell is GFI5.0 or YGLY8316 or strains set forth in U.S. Pat. No. 7,795,002 or Zha et al. Methods Mol Biol. 988: 31-43 (2013). See also International Patent Application Publication No. WO2013066765.

To make a composition comprising IL-12 heterodimeric Fc protein in which about 15% to 25% of the N-glycans are sialylated, a first composition of IL-12 heterodimeric Fc proteins in which greater than 25% of the N-glycans are sialylated is mixed with a second composition of IL-12 heterodimeric Fc protein s in which less than 10% of the N-glycans are sialylated at an appropriate ratio to produce a third composition in which 14.5%, 15%, 16%, 17%, 18%, 19%, 20%, 21%, 22%, 23%, 24%, or 25% of the N-glycans therein are sialylated. In cell culture systems, N-acetylmannosamine (ManNAc) is added to culture medium to allow for full sialylation and/or growth of the cells in culture (and products produced by these cells) and for production of recombinant sialylated proteins (See for example, Thomas et al., Biochim. Biophys. Acta. 846: 37-43 1985); Gu & Wang Biotechnol. Bioeng. 58: 642-648 (1998). Bork et al., FEBS Lett. 579: 5079-5083 (2005). Wang et al., Biotechnol. J. 14: e1800186 (2019); International Pat. Pub. WO2024059652; U.S. Pat. Pub. 20050084933, U.S. Pat. No. 7,645,609. Thus, the present invention provides IL-12 heterodimeric Fc proteins compositions in which about 14.5%, 15%, 16%, 17%, 18%, 19%, 20%, 21%, 22%, 23%, 24%, or 25% of the N-glycans therein are sialylated. In a further embodiment, the present invention provides IL-12 heterodimeric Fc proteins compositions in which 14.5% of the N-glycans therein are sialylated. In a further embodiment, the present invention provides IL-12 heterodimeric Fc proteins compositions in which 15% of the N-glycans therein are sialylated. In a further embodiment, the present invention provides IL-12 heterodimeric Fc proteins compositions in which 16% of the N-glycans therein are sialylated. In a further embodiment, the present invention provides IL-12 heterodimeric Fc proteins compositions in which 17% of the N-glycans therein are sialylated. In a further embodiment, the present invention provides IL-12 heterodimeric Fc proteins compositions in which 18% of the N-glycans therein are sialylated. In a further embodiment, the present invention provides IL-12 heterodimeric Fc proteins compositions in which 19% of the N-glycans therein are sialylated. In a further embodiment, the present invention provides IL-12 heterodimeric Fc proteins compositions in which 20% of the N-glycans therein are sialylated. In a further embodiment, the present invention provides IL-12 heterodimeric Fc proteins compositions in which 21% of the N-glycans therein are sialylated. In a further embodiment, the present invention provides IL-12 heterodimeric Fc proteins compositions in which 22% of the N-glycans therein are sialylated. In a further embodiment, the present invention provides IL-12 heterodimeric Fc proteins compositions in which 23% of the N-glycans therein are sialylated. In a further embodiment, the present invention provides IL-12 heterodimeric Fc proteins compositions in which 24% of the N-glycans therein are sialylated. In a further embodiment, the present invention provides IL-12 heterodimeric Fc proteins compositions in which 25% of the N-glycans therein are sialylated.

### VII. Pharmaceutical Compositions Comprising IL-12 Heterodimeric Fc Proteins of the Present Invention

The monovalent IL-12 heterodimeric Fc protein of the present invention disclosed herein may be provided in suitable pharmaceutical compositions comprising one or more monovalent IL-12 heterodimeric Fc proteins of the present invention and a pharmaceutically acceptable carrier. The carrier may be a diluent, adjuvant, excipient, or vehicle with which monovalent IL-12 heterodimeric Fc protein of the present invention is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the monovalent IL-12 heterodimeric Fc proteins of the invention in such pharmaceutical formulation may vary widely, i.e., from less than about 0.5%, usually to at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin, are described, for example, in Remington: The Science and Practice of Pharmacy, 21.sup.st Edition, Troy, D. B. ed., Lipincott Williams and Wilkins, Philadelphia, Pa. 2006, Part 5, Pharmaceutical Manufacturing pp 691-1092, see especially pp. 958-989.

The mode of administration of the monovalent IL-12 heterodimeric Fc protein of the present invention or pharmaceutical composition comprising the monovalent IL-12 heterodimeric Fc protein of the present invention thereof may be any suitable route such as parenteral administration, e.g., intradermal, intramuscular, intraperitoneal, intravenous, subcutaneous, pulmonary, transmucosal (oral, intranasal, intravaginal, rectal) or other means appreciated by the skilled artisan, as well known in the art.

The monovalent IL-12 heterodimeric Fc protein of the present invention may be administered to an individual (e.g., patient) by any suitable route, for example parentally by intravenous or systemic (i.v.) infusion or bolus injection, intramuscularly or subcutaneously, or intraperitoneally. An i.v. infusion may be given over for, example, 15, 30, 60, 90, 120, 180, or 240 minutes, or from 1, 2, 3, 4, 5, 6, 7, 8, 9, 10, 11 or 12 hours. In particular embodiments, the monovalent IL-12 heterodimeric Fc protein may be administered at or near a tumor or lymph node associated with the tumor.

The administration of the monovalent IL-12 heterodimeric Fc protein of the present invention or a pharmaceutical composition comprising the monovalent IL-12 heterodimeric Fc protein of the present invention may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose.

The monovalent IL-12 heterodimeric Fc protein of the present invention or a pharmaceutical composition comprising the monovalent IL-12 heterodimeric Fc protein of the present invention may be administered by maintenance therapy, such as, e.g., once a week for a period of 6 months or more.

The monovalent IL-12 heterodimeric Fc protein of the present invention or a pharmaceutical composition comprising the monovalent IL-12 heterodimeric Fc protein of the present invention may also be administered prophylactically in order to reduce the risk of developing cancer, delay the onset of the occurrence of an event in cancer progression, and/or reduce the risk of recurrence when a cancer is in remission. This may be especially useful in patients wherein it is difficult to locate a tumor that is known to be present due to other biological factors. In specific embodiments, the methods, compositions, and uses described herein for the monovalent IL-12 heterodimeric Fc proteins are for the prophylaxis of cancer or proliferative disease.

The monovalent IL-12 heterodimeric Fc protein of the present invention or pharmaceutical composition comprising the monovalent IL-12 heterodimeric Fc protein of the present invention may be lyophilized for storage and reconstituted in a suitable carrier prior to use. This technique has been shown to be effective with conventional protein preparations and well known lyophilization and reconstitution techniques can be employed.

### VIII. Combination Therapy Treatments

Combination therapies of the present invention comprising a monovalent IL-12 heterodimeric Fc protein of the present invention or pharmaceutical composition comprising the monovalent IL-12 heterodimeric Fc protein of the present invention and another therapeutic agent (e.g., small molecule or antibody) may be used for the treatment any proliferative disease, in particular, the treatment of cancer. In particular embodiments, the combination therapy of the present invention may be used to treat a proliferative disease selected from breast cancer (e.g., triple negative breast cancer), cervical cancer, colorectal cancer, esophageal cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

### IX. Combination Therapy Comprising a Monovalent IL-12 Heterodimeric Fc Protein of the Present Invention and Chemotherapy

The combination therapy of the present invention comprising a monovalent IL-12 heterodimeric Fc protein of the present invention or a pharmaceutical composition comprising the monovalent IL-12 heterodimeric Fc protein of the present invention may be administered to an individual having a cancer in combination with chemotherapy. The individual may undergo the chemotherapy at the same time the individual is undergoing the combination therapy of the present invention. The individual may undergo the combination therapy of the present invention after the individual has completed chemotherapy. The individual may be administered the chemotherapy after completion of the combination therapy. The combination therapy of the present invention may also be administered to an individual having recurrent or metastatic cancer with disease progression or relapse cancer and who is undergoing chemotherapy or who has completed chemotherapy.

The chemotherapy may include a chemotherapy agent selected from the group consisting of:
(i) alkylating agents, including but not limited to, bifunctional alkylators, cyclophosphamide, mechlorethamine, chlorambucil, and melphalan;
(ii) monofunctional alkylators, including but not limited to, dacarbazine, nitrosoureas, and temozolomide (oral dacarbazine);
(iii) anthracycline or alkylcycline;
(iv) cytoskeletal disruptors (taxanes), including but not limited to, paclitaxel, docetaxel, abraxane, and taxotere;
(v) epothilones, including but not limited to, ixabepilone, and utidelone; (vi) histone deacetylase inhibitors, including but not limited to, vorinostat, and romidepsin;
(vii) inhibitors of topoisomerase i, including but not limited to, irinotecan, and topotecan;
(viii) inhibitors of topoisomerase ii, including but not limited to, etoposide, teniposide, and tafluposide;
(ix) kinase inhibitors, including but not limited to, bortezomib, erlotinib, gefitinib, imatinib, vemurafenib, and vismodegib;
(x) nucleotide analogs and precursor analogs, including but not limited to, azacitidine, azathioprine, fluoropyrimidines (e.g., such as capecitabine, carmofur, doxifluridine, fluorouracil, and tegafur) cytarabine, gemcitabine, hydroxyurea, mercaptopurine, methotrexate, and tioguanine (formerly thioguanine);
(xi) peptide antibiotics, including but not limited to, bleomycin and actinomycin; a platinum-based agent, including but not limited to, carboplatin, cisplatin, and oxaliplatin;
(xii) retinoids, including but not limited to, tretinoin, alitretinoin, and bexarotene; and
(xiii) vinca alkaloids and derivatives, including but not limited to, vinblastine, vincristine, vindesine, and vinorelbine.

Selecting a dose of the chemotherapy agent for chemotherapy depends on several factors, including the serum or tissue turnover rate of the agent, the level of symptoms, the immunogenicity of the agent, and the accessibility of the target cells, tissue or organ in the individual being treated.

The dose of the additional therapeutic agent should be an amount that provides the intended therapeutic effect (i.e., treats the disease) and an acceptable level of side effects. Accordingly, the dose amount and dosing frequency of each additional therapeutic agent will depend in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dose regimen may be made by the clinician, e.g., using parameters or factors known or suspected in the art to affect treatment or predicted to affect treatment, and will depend, for example, on the individual's clinical history (e.g., previous therapy), the type and stage of the cancer to be treated and biomarkers of response to one or more of the therapeutic agents in the combination therapy.

Thus, the present invention contemplates embodiments of the combination therapy of the present invention that further includes a chemotherapy step comprising platinum-containing chemotherapy, e.g., pemetrexed and platinum chemotherapy or carboplatin and either paclitaxel or nab-paclitaxel. In particular embodiments, the combination therapy with a chemotherapy step may be used for treating at least NSCLC and HNSCC.

The combination therapy further in combination with a chemotherapy step may be used for the treatment of any proliferative disease, in particular, the treatment of cancer. In particular embodiments, the combination therapy of the present invention may be used to treat a proliferative disease selected from breast cancer (e.g., triple negative breast cancer), cervical cancer, colorectal cancer, esophageal cancer, lung cancer, non-Hodgkin's lymphoma, chronic lymphocytic lymphoma (CLL), Raji Burkitt lymphoma, oral squamous cell cancer, ovarian cancer, pancreatic cancer, prostate cancer, stomach cancer, thyroid cancer, urinary bladder cancer, glioma, oral cancer, gastric cancer, renal cancer, salivary duct cancer, anaplastic thyroid cancer, neuroendocrine, non-small cell lung cancer (NSCLC), squamous cell cancer of head and neck (SCCHN), colon cancer, sarcoma, esophageal cancer, cervical cancer, and uterine cancer.

### X. Combination Therapy Comprising a Monovalent IL-12 Heterodimeric Fc Protein and a Therapeutic Antibody

The monovalent IL-12 heterodimeric Fc protein of the present invention or a pharmaceutical composition comprising the monovalent IL-12 heterodimeric Fc protein of the present invention may be administered in combination with one or more therapeutic antibodies for the treatment of cancer or proliferative disease. The individual may undergo treatment with the therapeutic antibody at the same time the individual is undergoing the combination therapy of the present invention. The individual may undergo the combination therapy of the present invention after the individual has completed treatment with the therapeutic antibody. The individual may be administered the treatment with the therapeutic antibody after completion of the combination therapy. The combination therapy of the present invention may also be administered to an individual having recurrent or metastatic cancer with disease progression or relapsed cancer and who is undergoing chemotherapy or who has completed chemotherapy. In particular embodiments, the therapeutic antibody is a checkpoint inhibitor.

In particular embodiments, the therapeutic antibody targets the programmed death 1 receptor or ligand, PD-1 and PD-L1, respectively. Exemplary anti-PD-1 antibodies that may be used in a combination therapy with the monovalent IL-12 heterodimeric Fc proteins of the present invention disclosed herein include any antibody that binds PD-1 and inhibits PD-1 from binding PD-L1 and/or PD-L2 or binds PD-L1 or PD-L2 and inhibits it from binding PD-1. In a particular embodiment, the exemplary anti-PD-1 antibody is pembrolizumab (KEYTRUDA). In a particular embodiment, the exemplary anti-PD-1 antibody is nivolumab (OPDIVO). In a particular embodiment, the exemplary anti-PD-1 antibody is cemiplimab (LIBTAYO). In a particular embodiment, the exemplary anti-PD-L1 antibody is durvalumab (IMFINZI). In a particular embodiment, the exemplary anti-PD-L1 antibody is atezolizumab (TECENTRIQ). In a particular embodiment, the exemplary anti-PD-L1 antibody is avelumab (BAVENCIO).

In particular embodiments, the therapeutic antibody targets cytotoxic T-lymphocyte-associated protein 4 (CTLA-4). Exemplary anti-CTLA4 antibodies include tremelimumab (IgG2 monoclonal antibody available from Pfizer, formerly known as ticilimumab, CP-675,206); and ipilimumab (CTLA-4 antibody, also known as MDX-010, CAS No. 477202-00-9). Other exemplary anti-CTLA-4 antibodies are disclosed, e.g., in U.S. Pat. No. 5,811,097. In one embodiment, the anti-CTLA4 antibody is ipilimumab disclosed in, e.g., U.S. Pat. Nos. 5,811,097, 7,605,238, WOOO/32231 and WO97/20574.

### XI. Injection Device for Administering a Monovalent IL-12 Heterodimeric Fc Protein of the Present Invention

The present invention also provides an injection device comprising any one of the monovalent IL-12 heterodimeric Fc proteins of the present invention or a pharmaceutical composition comprising any one of the monovalent IL-12 heterodimeric Fc proteins of the present invention. An injection device is a device that introduces a substance into the body of a patient via a parenteral route, e.g., intramuscular, subcutaneous or intravenous. For example, an injection device may be a syringe (e.g., pre-filled with the pharmaceutical composition, such as an auto-injector) which, for example, includes a cylinder or barrel for holding fluid to be injected (e.g., comprising any one of the monovalent IL-12 heterodimeric Fc proteins of the present invention or pharmaceutical compositions comprising any one of the monovalent IL-12 heterodimeric Fc proteins of the present invention), a needle for piercing skin and/or blood vessels for injection of the fluid; and a plunger for pushing the fluid out of the cylinder and through the needle bore. In an embodiment of the invention, an injection device comprising any one of the monovalent IL-12 heterodimeric Fc proteins of the present invention or a pharmaceutical composition comprising any one of the monovalent IL-12 heterodimeric Fc proteins of the present invention is an intravenous (IV) injection device. Such a device includes a composition comprising said monovalent IL-12 heterodimeric Fc protein or a pharmaceutical composition in a cannula or trocar/needle which may be attached to a tube which may be attached to a bag or reservoir for holding fluid (e.g., saline; or lactated ringer solution comprising NaCl, sodium lactate, KCl, CaCl₂ and optionally including glucose) introduced into the body of the subject through the cannula or trocar/needle.

The monovalent IL-12 heterodimeric Fc proteins of the present invention or a pharmaceutical composition comprising the monovalent IL-12 heterodimeric Fc proteins of the present invention may, in an embodiment of the invention, be introduced into the device once the trocar and cannula are inserted into the vein of a subject and the trocar is removed from the inserted cannula. The IV device may, for example, be inserted into a peripheral vein (e.g., in the hand or arm); the superior vena cava or inferior vena cava, or within the right atrium of the heart (e.g., a central IV); or into a subclavian, internal jugular, or a femoral vein and, for example, advanced toward the heart until it reaches the superior vena cava or right atrium (e.g., a central venous line). In an embodiment of the invention, an injection device is an autoinjector; a jet injector or an external infusion pump. A jet injector uses a high-pressure narrow jet of liquid, which penetrates the epidermis to introduce the monovalent IL-12 heterodimeric Fc protein of the present invention or a pharmaceutical composition comprising the monovalent IL-12 heterodimeric Fc protein of the present invention to a patient's body. External infusion pumps are medical devices that deliver the monovalent IL-12 heterodimeric Fc protein of the present invention or a pharmaceutical composition comprising the monovalent IL-12 heterodimeric Fc protein of the present invention into a patient's body in controlled amounts. External infusion pumps may be powered electrically or mechanically. Different pumps operate in different ways, for example, a syringe pump holds fluid in the reservoir of a syringe, and a moveable piston controls fluid delivery, an elastomeric pump holds fluid in a stretchable balloon reservoir, and pressure from the elastic walls of the balloon drives fluid delivery. In a peristaltic pump, a set of rollers pinches down on a length of flexible tubing, pushing fluid forward. In a multi-channel pump, fluids can be delivered from multiple reservoirs at multiple rates.

### XII. Kits Comprising the Monovalent IL-12 Heterodimeric Fc Protein of the Present Invention or Composition Thereof

Further provided are kits comprising one or more components that include, but are not limited to, a monovalent IL-12 heterodimeric Fc protein of the present invention or a pharmaceutical composition comprising the monovalent IL-12 heterodimeric Fc protein of the present invention in association with one or more additional components including, but not limited to, a further therapeutic agent, as discussed herein. The monovalent IL-12 heterodimeric Fc protein of the present invention or a pharmaceutical composition comprising the monovalent IL-12 heterodimeric Fc protein of the present invention and/or the therapeutic agent can be formulated as a pure composition or in combination with a pharmaceutically acceptable carrier, in a pharmaceutical composition.

In one embodiment, the kit includes the monovalent IL-12 heterodimeric Fc protein of the present invention or a pharmaceutical composition comprising the monovalent IL-12 heterodimeric Fc protein of the present invention in one container (e.g., in a sterile glass or plastic vial) and a further therapeutic agent in another container (e.g., in a sterile glass or plastic vial).

In another embodiment, the kit comprises a combination of the invention, including the monovalent IL-12 heterodimeric Fc protein of the present invention or a pharmaceutical composition comprising the monovalent IL-12 heterodimeric Fc protein of the present invention in combination with one or more therapeutic agents formulated together, optionally, in a pharmaceutical composition, in a single, common container.

If the kit includes a pharmaceutical composition for parenteral administration to a subject, the kit can include a device for performing such administration. For example, the kit can include one or more hypodermic needles or other injection devices as discussed above. Thus, the present invention includes a kit comprising an injection device and the monovalent IL-12 heterodimeric Fc protein of the present invention or a pharmaceutical composition comprising the monovalent IL-12 heterodimeric Fc protein of the present invention, e.g., wherein the injection device includes the monovalent IL-12 heterodimeric Fc protein of the present invention or a pharmaceutical composition comprising the IL-12 variant of the present invention, or wherein the monovalent IL-12 heterodimeric Fc protein of the present invention or pharmaceutical composition comprising the monovalent IL-12 heterodimeric Fc protein of the present invention is in a separate vessel.

The kit can include a package insert including information concerning the pharmaceutical composition and dosage form in the kit. Generally, such information aids patients and physicians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

Additional specific embodiments of the present invention include the following.

Embodiment 1. A monovalent interleukin 12 (IL-12) heterodimeric Fc protein, comprising (a) an IL-12p35 subunit directly or indirectly linked to the N-terminus of a first immunoglobulin fragment crystallizable (Fc) region, and; (b) an IL-12p40 subunit directly or indirectly linked to the N-terminus of a second Fc region; wherein (i) the CH3 domains of the first Fc region and the second Fc region each comprise one or more amino acid substitutions that promote heterodimerization; (ii) at least one of the IL-12p35 and the IL-12p40 subunits of the monovalent IL-12 heterodimer Fc protein is a modified variant comprising one or more amino acid substitutions; and, (iii) the monomeric IL-12 heterodimeric Fc protein displays reduced potency compared to a monovalent IL-12 heterodimeric Fc protein comprising wild-type IL-12p35 and IL-12p40 subunits.

Embodiment 2. The monovalent IL-12 heterodimeric Fc protein of embodiment 1, wherein the potency is determined by measuring Signal Transducer and Activator of Transcription 4 (STAT4) protein induction in a cell-based assay comprising human embryo kidney (HEK) cells stably transfected with one or more nucleic acid molecules that express an IL-12 receptor and a STAT4-inducible reporter gene.

Embodiment 3. The monovalent IL-12 heterodimeric Fc protein of any one of embodiments 1-2, wherein the potency is reduced at least 10-fold.

Embodiment 4. The monovalent IL-12 heterodimeric Fc protein of any one of embodiments 1-3, wherein the potency is reduced at least 30-fold.

Embodiment 5. The monovalent IL-12 heterodimeric Fc protein of any one of embodiments 1-4, wherein the potency is reduced between 30-fold and 200-fold.

Embodiment 6. The monovalent IL-12 heterodimeric Fc protein of any one of embodiments 1-5, wherein the IL-12p35 subunit includes a deletion of the N-terminal hexapeptide RNLPVA (SEQ ID NO: 238).

Embodiment 7. The monovalent IL-12 heterodimeric Fc protein of any one of embodiments 1-5, wherein the IL-12p35 subunit is a modified variant comprising an I41S, E44R, D159K, D159R, Y161S, Y161T, or Y161A amino acid substitution, wherein the amino acid positions correspond to amino acid positions I41, E44, D159, and Y161 as set forth in SEQ ID NO: 133.

Embodiment 8. The monovalent IL-12 heterodimeric Fc protein of any one of embodiments 1-5, wherein the IL-12p35 subunit is a modified variant comprising a an I41S, E44R, D159K, D159R, Y161S, Y161T, or Y161A amino acid substitution amino acid substitution, wherein the amino acid positions correspond to amino acid positions I41, E44, D159, and Y161 as set forth in SEQ ID NO: 133, and wherein the IL-12p35 subunit further includes a deletion of the N-terminal hexapeptide RNLPVA (SEQ ID NO: 238).

Embodiment 9. The monovalent IL-12 heterodimeric Fc protein of any one of embodiments 1-5, wherein the IL-12p35 subunit is a modified variant comprising a an I41S, E44R, D159K, D159R, Y161S, Y161T, or Y161A amino acid substitution amino acid substitution, wherein the amino acid positions correspond to amino acid positions I41, E44, D159, and Y161 as set forth in SEQ ID NO: 133, and the IL-12p40 subunit comprises a wild-type amino acid sequence.

Embodiment 10. The monovalent IL-12 heterodimeric Fc protein of any one of embodiments 1-5, wherein the IL-12p35 subunit is a modified variant comprising a an I41S, E44R, D159K, D159R, Y161S, Y161T, or Y161A amino acid substitution amino acid substitution, wherein the amino acid positions correspond to amino acid positions I41, E44, D159, and Y161 as set forth in SEQ ID NO: 133, wherein the IL-12p35 subunit further comprises a deletion of the N-terminal hexapeptide RNLPVA (SEQ ID NO: 238), and the IL-12p40 subunit comprises a wild-type amino acid sequence.

Embodiment 11. The monovalent IL-12 heterodimeric Fc protein of any one of embodiments 1-5, wherein the IL12p40 subunit is a modified variant comprising a D87L, K104E, or K104D amino acid substitution, wherein the amino acid positions correspond to amino acid positions D87 and K104 as set forth in SEQ ID NO: 134.

Embodiment 12. The monovalent IL-12 heterodimeric Fc protein of any one of embodiments 1-5, wherein the IL12p40 subunit is a modified variant comprising a D87L, K104E, or K104D amino acid substitution, wherein the amino acid positions correspond to amino acid positions D87 and K104 as set forth in SEQ ID NO: 134, and the IL-12p35 subunit comprises a wild-type amino acid sequence or a wild-type amino acid sequence that further includes a deletion of the N-terminal hexapeptide RNLPVA (SEQ ID NO: 238).

Embodiment 13. The monovalent IL-12 heterodimeric Fc protein of any one of embodiments 1-5, wherein the IL-12p35 subunit is a modified variant comprising an I41S, E44R, D159K, D159R, Y161S, Y161T, or Y161A amino acid substitution amino acid substitution, wherein the amino acid positions correspond to amino acid positions I41, E44, D159, and Y161 as set forth in SEQ ID NO: 133, wherein the IL-12p35 subunit further includes a deletion of the N-terminal hexapeptide RNLPVA (SEQ ID NO: 238), and wherein the IL-12p40 subunit is a modified variant comprising a D87L, K104E, or K104D amino acid substitution, wherein the amino acid positions correspond to amino acid positions D87 and K104 as set forth in SEQ ID NO: 134.

Embodiment 14. The monovalent IL-12 heterodimeric Fc protein of any one of embodiments 1-5, wherein the IL-12p35 subunit is a modified variant an I41S, E44R, D159K, D159R, Y161S, Y161T, or Y161A amino acid substitution amino acid substitution, wherein the amino acid positions correspond to amino acid positions I41, E44, D159, and Y161 as set forth in SEQ ID NO: 133, and wherein the IL-12p40 subunit is a modified variant comprising amino acid substitutions selected from P143A/P215A, P143A/P215A/K260A/R261S/V303I, P143A/P215A/K260S/R261S/V303I, K260A/R261S/V303I, K260S/R261S/V303I, K260A/R261S, and K260S/R261S, wherein the amino acid positions correspond to amino acid positions P143, P215, K260, R261, and V303 as set forth in SEQ ID NO: 134.

Embodiment 15. The monovalent IL-12 heterodimeric Fc protein of any one of embodiments 1-5, wherein the IL-12p35 subunit is a modified variant comprising a Y161S or Y161T amino acid substitution, wherein the amino acid position corresponds to amino acid position Y161 as set forth in SEQ ID NO: 133, and the IL-12p40 subunit comprises a wild-type amino acid sequence or is a modified variant comprising a D87L, K104E, or K104D amino acid substitution, wherein the amino acid positions correspond to amino acid positions D87 and K104 as set forth in SEQ ID NO: 134.

Embodiment 16. The monovalent IL-12 heterodimeric Fc protein of any one of embodiments 1-5, wherein the IL-12 p35 subunit is a modified variant comprising an I41S, E44R, D159K, D159R, Y161S, Y161T, or Y161A amino acid substitution amino acid substitution, wherein the amino acid positions correspond to amino acid positions an 141, E44, D159, or Y161 amino acid substitution as set forth in SEQ ID NO: 133, wherein the IL-12p35 subunit further includes a deletion of the N-terminal hexapeptide RNLPVA (SEQ ID NO: 238), and wherein the p40 subunit is a modified variant comprising amino acid substitutions selected from P143A/P215A, P143A/P215A/K260A/R261S/V303I, P143A/P215A/K260S/R261S/V303I, K260A/R261S/V303I, K260S/R261S/V303I, K260A/R261S, and K260S/R261S, wherein the amino acid positions correspond to amino acid positions P143, P215, K260, R261, and V303 as set forth in SEQ ID NO: 134.

Embodiment 17. The monovalent IL-12 heterodimeric Fc protein of any one of embodiments 1-5, wherein the IL-12p35 subunit is a modified variant comprising a Y161S or Y161T amino acid substitution, wherein the amino acid position corresponds to amino acid position Y161 as set forth in SEQ ID NO: 133, wherein the IL-12p35 subunit further includes a deletion of the N-terminal hexapeptide RNLPVA (SEQ ID NO: 238), and the IL-12p40 subunit comprises a wild-type amino acid sequence or is a modified variant comprising a D87L, K104E, or K104D amino acid substitution, wherein the amino acid positions correspond to amino acid positions D87 and K104 as set forth in SEQ ID NO: 134.

Embodiment 18. The monovalent IL-12 heterodimeric Fc protein of any one of embodiments 1-5, wherein the IL-12p35 subunit is a modified variant comprising the amino acid sequence of SEQ ID NO: 140, 147, 148, 152, 153, or 154 and the IL12p40 subunit is a wild-type IL-12p40 subunit comprising the amino acid sequence set forth in SEQ ID NO: 134 or the IL-12p40 subunit is a modified variant comprising the amino acid sequence set forth in SEQ ID NO: 161, 166, 167, 192, 193, 194, 195, 196, 197, or 198.

Embodiment 19. The monovalent IL-12 heterodimeric Fc protein of any one of embodiments 1-5, wherein (i) the CH3 domain of the first Fc region comprises the amino acid substitutions S354C and T366W and the CH3 domain of the second Fc region comprises the amino acid substitutions Y349C, T366S, L368A, and Y407V, wherein the amino acid positions are according to the EU Index; (ii) the CH3 domain of the first Fc region comprises the amino acid substitutions Y349C, T366S, L368A, and Y407V and the CH3 domain of the second Fc region comprises the amino acid substitutions S354C and T366W, wherein the amino acid positions are according to the EU Index; (iii) the CH3 domain of the first Fc region comprises the amino acid substitutions K409W and the CH3 domain of the second Fc region comprises the amino acid substitutions D399V and F405T, wherein the amino acid positions are according to the EU Index; (iv) the CH3 domain of the first Fc region comprises the amino acid substitutions D399V and F405T and the CH3 domain of the second Fc region comprises the amino acid substitution K409W, wherein the amino acid positions are according to the EU Index; (v) the CH3 domain of the first Fc region comprises the amino acid substitution K357W and the CH3 domain of the second Fc region comprises the amino acid substitution Y349S, wherein the amino acid positions are according to the EU Index; or, (vi) the CH3 domain of the first Fc region comprises the amino acid substitution Y349S and the CH3 domain of the second Fc region comprises the amino acid substitution K357W, wherein the amino acid positions are according to the EU Index.

Embodiment 20. The monovalent IL-12 heterodimeric Fc protein of any one of embodiments 1-5, wherein (a)(i) the IL-12p35 subunit is a modified variant comprising an I41S, E44R, D159K, D159R, Y161S, Y161T, or Y161A amino acid substitution amino acid substitution directly or indirectly linked to the N-terminus of a first Fc region comprising the amino acid substitutions S354C and T366W, wherein the Fc amino acid positions are according to the EU Index and the amino acid positions of the IL-12p35 subunit variant correspond to amino acid positions I41, E44, D159, or Y161 as set forth in SEQ ID NO: 133; and, (a)(ii) the IL-12p40 subunit is directly or indirectly linked to the N-terminus of a second Fc region comprising the amino acid substitutions Y349C, T366S, L368A, and Y407V, wherein the amino acid positions are according to the EU Index and the IL-12p40 subunit is wild-type; or (b)(i) the IL-12 p40 subunit is directly or indirectly linked to the N-terminus of a first Fc region comprising the amino acid substitutions S354C and T366W, wherein the amino acid positions are according to the EU Index and the IL-12p40 subunit is wild-type; and (b)(ii) the IL-12p35 subunit is a modified variant comprising an I41S, E44R, D159K, D159R, Y161S, Y161T, or Y161A amino acid substitution amino acid substitution directly or indirectly linked to the N-terminus of a second Fc region comprising the amino acid substitutions Y349C, T366S, L368A, and Y407V wherein the Fc amino acid positions are according to the EU Index and the amino acid positions of the IL-12p35 subunit variant correspond to amino acid positions I41, E44, D159, or Y161 as set forth in SEQ ID NO: 133.

Embodiment 21. The monovalent IL-12 heterodimeric Fc protein of any one of embodiments 1-20, wherein the CH3 domains of the first Fc region and the second Fc region each comprise one or more amino acid substitutions that increase serum half-life of the monovalent IL-12 heterodimeric Fc protein compared to the half-life of a monovalent IL-12 heterodimeric Fc protein comprising a wild-type Fc.

Embodiment 22. The monovalent IL-12 heterodimeric Fc protein of embodiment 21, wherein the CH3 domains of the first Fc region and the second Fc region each comprise amino acid substitutions selected from the group consisting of (i) M252Y, S254T, and T256E; (ii) M428L and N434S; (iii) L309D, Q311H, and N434S; and (iv) H433K and N434F, wherein the amino acid positions are according to the Eu numbering scheme.

Embodiment 23. The monovalent IL-12 heterodimeric Fc protein of any one of embodiments 1-22, wherein the CH3 domains of the first Fc region and the second Fc region each comprise one or more amino acid substitutions that reduce an Fc region effector function.

Embodiment 24. The monovalent IL-12 heterodimeric Fc protein of embodiment 23, wherein the CH3 domains of the first Fc region and the second Fc region each comprise amino acid substitutions selected from the group consisting of (i) L234A, L235A, and D265S; (ii) L234A, L235A, and P329G; (iii) L234A and L235A; (iv) H268Q, V309L, A330S, and P331S; (v) V234A, G237A, P338S, H268A, and V309L; and (vi) N297X wherein X is A, Q, or G; wherein the amino acid positions are according to the Eu numbering scheme.

Embodiment 25. The monovalent IL-12 heterodimeric Fc protein of any one of embodiments 1-24, wherein the IL-12p35 subunit or modified variant thereof is indirectly linked to the N-terminus of the first Fc region via a first peptide or non-peptidyl polymer and the wherein the IL-12p40 subunit or modified variant thereof is indirectly linked to the N-terminus of the second Fc region via a second peptide or non-peptidyl polymer.

Embodiment 26. The monovalent IL-12 heterodimeric Fc protein of embodiment 1, wherein the first peptide linker comprises the amino acid sequence GGGGSGGGGSGGGGS (SEQ ID NO: 206) and the second peptide linker comprises the amino acid sequence GGGG (SEQ ID NO: 202).

Embodiment 27. The monovalent IL-12 heterodimeric Fc protein of embodiment 1, wherein the first and second non-peptidyl polymer are independently selected from polyethylene glycol, polypropylene glycol, copolymers of ethylene glycol and propylene glycol, polyoxyethylated polyols, polyvinyl alcohol, polysaccharides, polyvinyl ethyl ether, polylactic acid, polylacticglycolic acid, lipid polymers, hyaluronic acid, and combinations thereof.

Embodiment 28. The monovalent IL-12 heterodimeric Fc protein of embodiment 1, wherein IL-12p35 subunit comprises the amino acid sequence set forth in SEQ ID NO: 239 and the IL-12p40 subunit comprises the amino acid sequence set forth in SEQ ID NO: 134.

Embodiment 29. A monovalent IL-12 heterodimeric Fc protein comprising (a) an IL-12p35 polypeptide comprising an amino acid substitution selected from the group consisting of SEQ ID NO: 133, SEQ ID NO: 140, SEQ ID NO: 148, SEQ ID NO: 149, SEQ ID NO: 152, SEQ ID NO: 153, SEQ ID NO: 154, SEQ ID NO: 239, SEQ ID NO: 266, SEQ ID NO: 274, SEQ ID NO: 275, SEQ ID NO: 278, SEQ ID NO: 279, and SEQ ID NO: 280 linked to an Fc protein comprising an amino acid sequence selected from the group consisting of SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, and SEQ ID NO: 412; and (b) an IL-12p40 polypeptide comprising a wild-type amino acid sequence or an amino acid substitution selected from the group consisting of SEQ ID NO: 134, SEQ ID NO: 161, SEQ ID NO:166, and SEQ ID NO: 167 linked to an Fc protein comprising an amino acid sequence selected from the group consisting of SEQ ID NO: 209, SEQ ID NO: 211, SEQ ID NO: 213, SEQ ID NO: 215, SEQ ID NO: SEQ ID NO: 217, SEQ ID NO: 219, SEQ ID NO: 221, SEQ ID NO:397, SEQ ID NO: 399, SEQ ID NO: 401, SEQ ID NO: 403, SEQ ID NO: 405, SEQ ID NO: 407, SEQ ID NO: 409, SEQ ID NO: 411, and SEQ ID NO: 413.

Embodiment 30. A monovalent IL-12 heterodimeric Fc protein comprising: (a) an IL-12p35 polypeptide comprising an amino acid substitution selected from the group consisting of SEQ ID NO: 133, SEQ ID NO: 140, SEQ ID NO: 148, SEQ ID NO: 149, SEQ ID NO: 152, SEQ ID NO: 153, SEQ ID NO: 154, SEQ ID NO: 239, SEQ ID NO: 266, SEQ ID NO: 274, SEQ ID NO: 275, SEQ ID NO: 278, SEQ ID NO: 279, and SEQ ID NO: 280 linked to an Fc protein comprising an amino acid sequence selected from the group consisting of SEQ ID NO: 209, SEQ ID NO: 211, SEQ ID NO: 213, SEQ ID NO: 215, SEQ ID NO: SEQ ID NO: 217, SEQ ID NO: 219, SEQ ID NO: 221, SEQ ID NO:397, SEQ ID NO: 399, SEQ ID NO: 401, SEQ ID NO: 403, SEQ ID NO: 405, SEQ ID NO: 407, SEQ ID NO: 409, SEQ ID NO: 411, and SEQ ID NO: 413; and (b) an IL-12p40 polypeptide comprising a wild-type amino acid sequence or an amino acid substitution selected from the group consisting of SEQ ID NO: 134, SEQ ID NO: 161, SEQ ID NO:166, and SEQ ID NO: 167 linked to an Fc protein comprising an amino acid sequence selected from the group consisting of SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, and SEQ ID NO: 412.

Embodiment 31. A monovalent IL-12 heterodimeric Fc protein comprising: (a) a first polypeptide comprising the amino acid sequence set forth in SEQ ID NO: 8 and a second polypeptide comprising the amino acid sequence set forth in SEQ ID NO: 2; (b) a first polypeptide comprising the amino acid sequence set forth in SEQ ID NO: 15 and a second polypeptide comprising the amino acid sequence set forth in SEQ ID NO: 2; (c) a first polypeptide comprising the amino acid sequence set forth in SEQ ID NO: 16 and a second polypeptide comprising the amino acid sequence set forth in SEQ ID NO: 2; (d) a first polypeptide comprising the amino acid sequence set forth in SEQ ID NO: 17 and a second polypeptide comprising the amino acid sequence set forth in SEQ ID NO: 2; (e) a first polypeptide comprising the amino acid sequence set forth in SEQ ID NO: 20 and a second polypeptide comprising the amino acid sequence set forth in SEQ ID NO: 2; (f) a first polypeptide comprising the amino acid sequence set forth in SEQ ID NO: 21 and a second polypeptide comprising the amino acid sequence set forth in SEQ ID NO: 2; or (g) a first polypeptide comprising the amino acid sequence set forth in SEQ ID NO: 22 and a second polypeptide comprising the amino acid sequence set forth in SEQ ID NO: 2.

Embodiment 32. A monovalent IL-12 heterodimeric Fc protein comprising: (a) a first polypeptide comprising the amino acid sequence set forth in SEQ ID NO: 74 and a second polypeptide comprising the amino acid sequence set forth in SEQ ID NO: 68; (b) a first polypeptide comprising the amino acid sequence set forth in SEQ ID NO: 81 and a second polypeptide comprising the amino acid sequence set forth in SEQ ID NO: 68; (c) a first polypeptide comprising the amino acid sequence set forth in SEQ ID NO: 82 and a second polypeptide comprising the amino acid sequence set forth in SEQ ID NO: 68; (d) a first polypeptide comprising the amino acid sequence set forth in SEQ ID NO: 83 and a second polypeptide comprising the amino acid sequence set forth in SEQ ID NO: 68; (e) a first polypeptide comprising the amino acid sequence set forth in SEQ ID NO: 86 and a second polypeptide comprising the amino acid sequence set forth in SEQ ID NO: 68; (f) a first polypeptide comprising the amino acid sequence set forth in SEQ ID NO: 87 and a second polypeptide comprising the amino acid sequence set forth in SEQ ID NO: 68; or (g) a first polypeptide comprising the amino acid sequence set forth in SEQ ID NO: 88 and a second polypeptide comprising the amino acid sequence set forth in SEQ ID NO: 68.

Embodiment 33. A pharmaceutical composition comprising the monovalent IL-12 heterodimeric Fc protein of any one of embodiments 1-32 and a pharmaceutical carrier or diluent.

Embodiment 34. Use of the monovalent IL-12 heterodimeric Fc protein of any one of embodiments 1-32 or the pharmaceutical composition of embodiment 33 for the manufacture of a medicament for the treatment of a cancer.

Embodiment 35. The monovalent IL-12 heterodimeric Fc protein of any one of embodiments 1-32 or the pharmaceutical composition of embodiment 33 for the manufacture of a medicament for the treatment of a cancer.

Embodiment 36. A method for treating a cancer comprising: administering a therapeutically effective amount of the monovalent IL-12 heterodimeric Fc protein of any one of embodiments 1-32 or the pharmaceutical composition of embodiment 33 to an individual in need thereof.

Embodiment 37. A therapy for treating cancer wherein a monovalent IL-12 heterodimeric Fc protein of any one of embodiments 1-32 or the pharmaceutical composition of embodiment 33 is provided in combination with an anti-cancer treatment selected from radiation treatment, chemotherapy treatment, immune checkpoint inhibitor treatment, or antibody-drug conjugate treatment.

Embodiment 38. A method for treating a cancer comprising: administering a therapeutically effective amount of the monovalent IL-12 heterodimeric Fc protein of any one of embodiments 1-32 or the pharmaceutical composition of embodiment 33 and an anti-cancer treatment selected from radiation treatment, chemotherapy treatment, immune checkpoint inhibitor treatment, or antibody-drug conjugate treatment to an individual in need thereof.

Embodiment 39. A monovalent interleukin 12 (IL-12) heterodimeric Fc protein, comprising (i) an IL-12p35 subunit fused to the N-terminus of a first immunoglobulin fragment crystallizable (Fc) region, and; (ii) an IL-12p40 subunit fused to the N-terminus of a second Fc region; wherein the first Fc region and the second Fc region each comprise one or more amino acid substitutions that promote heterodimerization, and; wherein the IL-12p35 subunit of the monovalent IL-12 heterodimer Fc protein comprises one or more amino acid substitutions that reduce the potency of the monovalent IL-12 heterodimeric Fc protein compared to a monovalent IL-12 heterodimeric Fc protein comprising wild-type IL-12p35 and IL-12p40 subunits.

Embodiment 40. The monovalent IL-12 heterodimeric Fc protein of embodiment 39, wherein the potency is determined by measuring Signal Transducer and Activator of Transcription 4 (STAT4) protein induction in a cell-based assay comprising human embryo kidney (HEK) cells stably transfected with one or more nucleic acid molecules that express an IL-12 receptor and a STAT4-inducible reporter gene.

Embodiment 41. The monovalent IL-12 heterodimeric Fc protein of any one of embodiments 39-40, wherein the IL-12p35 subunit comprises the amino acid sequence set forth in SEQ ID NO: 140, 147, 148, 152, 153, or 154.

Embodiment 42. The monovalent IL-12 heterodimeric Fc protein of any one of embodiments 39-41, wherein the IL-12p40 subunit comprises the amino acid sequence set forth in SEQ ID NO: 134, 161, 166, 167, 192, 193, 194, 195, 196, 197, or 198.

Embodiment 43. The monovalent IL-12 heterodimeric Fc protein of any one of embodiments 39-42, wherein the IL-12p35 subunit comprises the amino acid sequence set forth in SEQ ID NO: 140, 147, 148, 152, 153, or 154 and the IL-12p40 subunit comprises the amino acid sequence set forth in SEQ ID NO: 134, 161, 166, 167, 192, 193, 194, 195, 196, 197, or 198.

Embodiment 44. The monovalent IL-12 heterodimeric Fc protein of any one of embodiments 39-43, wherein the IL-12p35 subunit comprises the amino acid sequence set forth in SEQ ID NO: 140, 147, 148, 152, 153, or 154 and the IL-12p40 subunit comprises the amino acid sequence set forth in SEQ ID NO: 134, 192, 193, 194, 195, 196, 197, or 198.

Embodiment 45. The monovalent IL-12 heterodimeric Fc protein of embodiment 39, wherein (a) the first Fc region comprises the amino acid sequence set forth in SEQ ID NO: 208 and the second Fc region comprises the amino acid sequence set forth in SEQ ID NO: 209; (b) the first Fc region comprises the amino acid sequence set forth in SEQ ID NO: 210 and the second Fc region comprises the amino acid sequence set forth in SEQ ID NO: 211; (c) the first Fc region comprises the amino acid sequence set forth in SEQ ID NO: 212 and the second Fc region comprises the amino acid sequence set forth in SEQ ID NO: 213; (d) the first Fc region comprises the amino acid sequence set forth in SEQ ID NO: 214 and the second Fc region comprises the amino acid sequence set forth in SEQ ID NO: 215; (e) the first Fc region comprises the amino acid sequence set forth in SEQ ID NO: 216 and the second Fc region comprises the amino acid sequence set forth in SEQ ID NO: 217; (f) the first Fc region comprises the amino acid sequence set forth in SEQ ID NO: 218 and the second Fc region comprises the amino acid sequence set forth in SEQ ID NO: 219; (g) the first Fc region comprises the amino acid sequence set forth in SEQ ID NO: 396 and the second Fc region comprises the amino acid sequence set forth in SEQ ID NO: 397; (h) the first Fc region comprises the amino acid sequence set forth in SEQ ID NO: 398 and the second Fc region comprises the amino acid sequence set forth in SEQ ID NO: 399; (i) the first Fc region comprises the amino acid sequence set forth in SEQ ID NO: 400 and the second Fc region comprises the amino acid sequence set forth in SEQ ID NO: 401; (j) the first Fc region comprises the amino acid sequence set forth in SEQ ID NO: 402 and the second Fc region comprises the amino acid sequence set forth in SEQ ID NO: 403; (k) the first Fc region comprises the amino acid sequence set forth in SEQ ID NO: 404 and the second Fc region comprises the amino acid sequence set forth in SEQ ID NO: 405; (l) the first Fc region comprises the amino acid sequence set forth in SEQ ID NO: 406 and the second Fc region comprises the amino acid sequence set forth in SEQ ID NO: 407; (m) the first Fc region comprises the amino acid sequence set forth in SEQ ID NO: 408 and the second Fc region comprises the amino acid sequence set forth in SEQ ID NO: 409; (n) the first Fc region comprises the amino acid sequence set forth in SEQ ID NO: 410 and the second Fc region comprises the amino acid sequence set forth in SEQ ID NO: 411; or (o) the first Fc region comprises the amino acid sequence set forth in SEQ ID NO: 412 and the second Fc region comprises the amino acid sequence set forth in SEQ ID NO: 413.

Embodiment 46. The monovalent IL-12 heterodimeric Fc protein of embodiment 39, wherein (a) the first Fc region comprises the amino acid sequence set forth in SEQ ID NO: 209 and the second Fc region comprises the amino acid sequence set forth in SEQ ID NO: 208; (b) the first Fc region comprises the amino acid sequence set forth in SEQ ID NO: 211 and the second Fc region comprises the amino acid sequence set forth in SEQ ID NO: 210; (c) the first Fc region comprises the amino acid sequence set forth in SEQ ID NO: 213 and the second Fc region comprises the amino acid sequence set forth in SEQ ID NO: 212; (d) the first Fc region comprises the amino acid sequence set forth in SEQ ID NO: 215 and the second Fc region comprises the amino acid sequence set forth in SEQ ID NO: 214; (e) the first Fc region comprises the amino acid sequence set forth in SEQ ID NO: 217 and the second Fc region comprises the amino acid sequence set forth in SEQ ID NO: 216; (f) the first Fc region comprises the amino acid sequence set forth in SEQ ID NO: 219 and the second Fc region comprises the amino acid sequence set forth in SEQ ID NO: 218; (g) the first Fc region comprises the amino acid sequence set forth in SEQ ID NO: 397 and the second Fc region comprises the amino acid sequence set forth in SEQ ID NO: 396; (h) the first Fc region comprises the amino acid sequence set forth in SEQ ID NO: 399 and the second Fc region comprises the amino acid sequence set forth in SEQ ID NO: 398; (i) the first Fc region comprises the amino acid sequence set forth in SEQ ID NO: 401 and the second Fc region comprises the amino acid sequence set forth in SEQ ID NO: 400; (j) the first Fc region comprises the amino acid sequence set forth in SEQ ID NO: 403 and the second Fc region comprises the amino acid sequence set forth in SEQ ID NO: 402; (k) the first Fc region comprises the amino acid sequence set forth in SEQ ID NO: 405 and the second Fc region comprises the amino acid sequence set forth in SEQ ID NO: 404; (l) the first Fc region comprises the amino acid sequence set forth in SEQ ID NO: 407 and the second Fc region comprises the amino acid sequence set forth in SEQ ID NO: 406; (m) the first Fc region comprises the amino acid sequence set forth in SEQ ID NO: 409 and the second Fc region comprises the amino acid sequence set forth in SEQ ID NO: 408; (n) the first Fc region comprises the amino acid sequence set forth in SEQ ID NO: 411 and the second Fc region comprises the amino acid sequence set forth in SEQ ID NO: 410; or (o) the first Fc region comprises the amino acid sequence set forth in SEQ ID NO: 413 and the second Fc region comprises the amino acid sequence set forth in SEQ ID NO: 412.

Embodiment 47. The monovalent IL-12 heterodimeric Fc protein of embodiment 39, wherein the IL-12p35 subunit is fused to the N-terminus of the first Fc region via a peptide linker comprising the amino acid sequence set forth in SEQ ID NO: 204, 205, 206, or 207; and, the IL-12p40 subunit is fused to the N-terminus of the second Fc region via a peptide linker comprising the amino acid sequence set forth in SEQ ID NO: 202.

Embodiment 48. The monovalent IL-12 heterodimeric Fc protein of embodiment 39, wherein the IL-12p35 subunit fused to the N-terminus of the first Fc region is a first polypeptide comprising the amino acid sequence set forth in SEQ ID NO: 74, 81, 82, 83, 86, 87, or 88; and, the IL-12p40 subunit fused to the N-terminus of the second Fc region is a second polypeptide comprising the amino acid sequence set forth in SEQ ID NO: 68.

Embodiment 49. The monovalent IL-12 heterodimeric Fc protein of embodiment 39, wherein the IL-12p35 subunit fused to the N-terminus of the first Fc region is a first polypeptide comprising the amino acid sequence set forth in SEQ ID NO: 8, 15, 16, 17, 20, 21, or 22; and, the IL-12p40 subunit fused to the N-terminus of the second Fc region is a second polypeptide comprising the amino acid sequence set forth in SEQ ID NO: 2.

Embodiment 50. A pharmaceutical composition comprising the monovalent IL-12 heterodimeric Fc protein of any one of embodiments 39 to 49 and a pharmaceutical carrier or diluent.

Embodiment 51. Use of the monovalent IL-12 heterodimeric Fc protein of any one of embodiments 39 to 49 or the pharmaceutical composition of embodiment 50 for the manufacture of a medicament for the treatment of a cancer.

Embodiment 52. The monovalent IL-12 heterodimeric Fc protein of any one of embodiments 39 to 49 or the pharmaceutical composition of embodiment 50 for the manufacture of a medicament for the treatment of a cancer.

Embodiment 53. A method for treating a cancer comprising: administering a therapeutically effective amount of the monovalent IL-12 heterodimeric Fc protein of any one of embodiments 39 to 49 or the pharmaceutical composition of embodiment 50 to an individual in need thereof.

Embodiment 54. A therapy for treating cancer wherein a monovalent IL-12 heterodimeric Fc protein of any one of embodiments 39 to 49 or the pharmaceutical composition of embodiment 50 is provided in combination with an anti-cancer treatment selected from radiation treatment, chemotherapy treatment, immune checkpoint inhibitor treatment, or antibody-drug conjugate treatment.

Embodiment 55. A method for treating a cancer comprising: administering a therapeutically effective amount of the monovalent IL-12 heterodimeric Fc protein of any one of embodiments 39 to 49 or the pharmaceutical composition of embodiment 50 and an anti-cancer treatment selected from radiation treatment, chemotherapy treatment, immune checkpoint inhibitor treatment, or antibody-drug conjugate treatment to an individual in need thereof.

Embodiment 56. A method for producing a monovalent IL-12 heterodimeric Fc protein, comprising: (a) providing a first nucleic acid molecule encoding a first polypeptide comprising an IL-12p35 subunit fused to the N-terminus of a first immunoglobulin fragment crystallizable (Fc) region and a second nucleic acid molecule encoding a second polypeptide comprising an IL-12p40 subunit fused to the N-terminus of a second Fc region, wherein the first Fc region and the second Fc region each comprise one or more amino acid substitutions that promote heterodimerization, and wherein the IL-12p35 subunit of the monovalent IL-12 heterodimer Fc protein comprises one or more amino acid substitutions that reduce the potency of the monovalent IL-12 heterodimeric Fc protein compared to a monovalent IL-12 heterodimeric Fc protein comprising wild-type IL-12p35 and IL-12p40 subunits; (b) transfecting a host cell with the first nucleic acid molecule and the second nucleic acid molecule to produce a transfected host cell; (c) cultivating the host cell in a culture medium under conditions for the production of the first polypeptide and the second polypeptide, which are excreted into the culture medium and associated into a monovalent IL-12 heterodimeric Fc protein comprising a first polypeptide and a second polypeptide; and (d) recovering the monovalent IL-12 heterodimeric Fc proteins from the culture medium.

Embodiment 57. The method of embodiment 56, wherein the first polypeptide comprises the amino acid sequence set forth in SEQ ID NO: 74, 81, 82, 83, 86, 87, or 88 and the second polypeptide comprises the amino acid sequence set forth in SEQ ID NO: 68.

Embodiment 58. The method of embodiment 56, wherein the first polypeptide comprises the amino acid sequence set forth in SEQ ID NO: 8, 15, 16, 17, 20, 21, or 22 and the second polypeptide comprises the amino acid sequence set forth in SEQ ID NO: 2.

Embodiment 59. The method of embodiment 56, wherein the first nucleic acid molecule comprises the nucleotide sequence set forth in SEQ ID NO: 223, 224, 25, 226, 227, 228, or 229 and the second nucleic acid molecule comprises the nucleotide sequence set forth in SEQ ID NO: 230.

Embodiment 60. The method of embodiment 56, wherein the first nucleic acid molecule comprises the nucleotide sequence set forth in SEQ ID NO: 234, 235, 236, 240, 241, 242, or 243 and the second nucleic acid molecule comprises the nucleotide sequence set forth in SEQ ID NO: 237.

Embodiment 61. A polypeptide comprising the amino acid sequence set forth in SEQ ID NO: 1, 2, 3, 4, 5, 6, 7, 8, 9, 10, 11, 12 13, 14 15, 16 17, 18, 19, 20, 21, 22, 23, 24 25 26, 27, 28, 29, 30, 31, 32, 33, 34, 35 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, or 66.

Embodiment 62. A polypeptide comprising the amino acid sequence set forth in SEQ ID NO: 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127,128, 129, 130, 131, or 132.

Embodiment 63. A polypeptide comprising the amino acid sequence set forth in SEQ ID NO: 133, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, or 198.

Embodiment 64. A polypeptide comprising the amino acid set forth in SEQ ID NO: 261, 262, 263, 264, 265, 266, 267, 268, 269, 270, 271, 272, 273, 274, 275, 276, 277, 278, 278, 279, 280, 281, 282, or 283.

Embodiment 65. A polypeptide comprising the amino acid set forth in SEQ ID NO: 284, 285, 286, 287, 288, 289, 290, 291, 292, 293, 294, 295, 296, 297, 298, 299, 300, 301, 302, or 303.

Embodiment 65. A polypeptide comprising the amino acid set forth in SEQ ID NO: 304, 305, 306, 307, 308, 309, 310, 311, 312, 313, 314, 315, 316, 317, 318, 319, 320, 321, 322, 323, 324, 325, or 326.

Embodiment 66. A polypeptide comprising the amino acid set forth in SEQ ID NO: 327, 328, 329, 330, 331, 332, 333, 334, 335,336, 337, 338, 339, 340, 341, 342, 343, 344, 345, or 346.

Embodiment 67. A polypeptide comprising the amino acid set forth in SEQ ID NO: 347, 348, 349, 350, 351, 352, 353, 354, 356, 357, 358, 359, 360, 361, 362, 363, 364, 365, 366, 367, 368, or 369.

Embodiment 68. A polypeptide comprising the amino acid set forth in SEQ ID NO: 370, 371, 372, 373, 374, 375, 376, 377, 378, 379, 380, 381, 382, 383, 384, 385, 386, 387, 388, or 389.

Embodiment 69. A nucleic acid molecule comprising the nucleotide sequence set forth in SEQ ID NO: 223, 224, 225, 226, 227, 228, 229, 230, 231, 232, 233, 249, or 250.

Embodiment 70. A nucleic acid molecule comprising the nucleotide sequence set forth in SEQ ID NO: 234, 235, 236, 237, 240, 241, 242, 243, 244, 245, 246, 247, or 248.

Embodiment 72. A nucleic acid molecule comprising the nucleotide sequence set forth in SEQ ID NO: 251, 252, 253, 254, 255, 256, 257, 258, 259, or 260.

Embodiment 73. A monovalent IL-12 heterodimeric Fc protein comprising a polypeptide of any one of Embodiments 61 to 68.

Embodiment 74. A monovalent IL-12 heterodimeric Fc protein comprising an IL-12p35 polypeptide and an IL-12p40 polypeptide of any one of embodiments 61 to 68.

Embodiment 75. A composition comprising a monovalent IL-12 heterodimeric Fc protein disclosed herein wherein 14.5% to 25% of the N-glycans in the composition are sialylated.

Embodiment 76. A composition comprising a monovalent IL-12 heterodimeric Fc protein disclosed herein wherein 15% of the N-glycans in the composition are sialylated.

Embodiment 77. A composition comprising a monovalent IL-12 heterodimeric Fc protein disclosed herein wherein 20% of the N-glycans in the composition are sialylated.

Embodiment 78. A composition comprising a monovalent IL-12 heterodimeric Fc protein disclosed herein wherein 25% of the N-glycans in the composition are sialylated.

The following examples are intended to promote a further understanding of the present invention.

### Example 1

Identification of Novel Potency Reduced IL-12-Fc Variants with Varying Degrees of Reduction. Protein based approach was selected to leverage vast historical/published IL-12 efficacy/toxicity data in both human and pre-clinical models. Cytokines in nature are produced locally with inherently short pharmacokinetics (PK). Half-life extension through a combination of Fc fusion and potency reduction was considered to increase the IL-12 therapeutic window. Fc fusion improves half-life and/or pharmacodynamic (PD)/efficacy response for IL-12 in human, non-human primate (NHP), and syngeneic mouse tumor models. Potency reduced Fc-tagged cytokines show improved therapeutic windows (improved PD and reduced toxic) vs. WT potency Fc-tagged cytokine in NHP. Various 100×-1000× potency reduced IL-12-Fc disclosed in WO2020072821 were shown to be able to mount strong anti-tumor activity while inducing activation and proliferation of T cells, PD1 expression, and intra-tumoral PD response. The strategy herein was to screen 79 single point and combination mutations on the IL-12p35 and Il-12p40 subunits in three rounds of engineering.

Initial round of potency-reduced monovalent IL-12 heterodimeric Fc protein generation involved single amino acid mutation on either IL-12p35 or Il-12p40 subunit. The commercially available engineered human embryo kidney (HEK) Signal Transducer And Activator Of Transcription 4 (STAT4) reporter cell line (HEK-Blue™, Invivogen (San Diego, CA): catalog #hkb-i112) was used to select desired candidates. HEK-Blue™ IL-12 cells were generated by stably introducing the human genes for the IL-12 receptor (IL-12R) and the genes of the IL-12 signaling pathway into the HEK 293 cell line. In addition, these cells express a STAT4-inducible Secreted Embryonic Alkaline Phosphatase (SEAP) reporter gene. The binding of IL-12 to the IL-12R on the surface of HEK-Blue™ IL-12 cells triggers a signaling cascade leading to the activation of STAT-4 with the subsequent production of SEAP. Detection of SEAP in the supernatant of HEK-Blue™ IL-12 cells can be assessed using QUANTI-Blue™ Solution.

Expression vectors, each comprising an open reading frame (ORF) encoding a monovalent IL-12 heterodimeric Fc protein, were separately transfected into CHO cells. For each variant, a first expression vector ORF encoding IL-12p35-Fc (LALADS-KIH-Knob) variants and a second expression vector ORF encoding IL-12p40-Fc (LALADS-KIH-Hole) in which the IL-12p40 subunit were co-transfected into CHO cells to produce monovalent IL-12 heterodimeric Fc proteins. Representative monovalent IL-12 heterodimeric Fc proteins are shown in the following tables. Table 49 provides the amino acid sequences for various monovalent IL-12 heterodimeric Fc proteins comprising a mutated IL-12p35 subunit and a wild-type IL-12p40 subunit. Table 50 provides the amino acid sequences for various monovalent IL-12 heterodimeric Fc proteins comprising a wild-type IL-12p35 subunit and a mutated IL-12p40 subunit. Table 51 provides the amino acid sequences for various monovalent IL-12 heterodimeric Fc proteins comprising a mutated IL-12p35 subunit and a mutated IL-12p40 subunit.

TABLE 49

| | | IL-12p35-Fc (LALADS-KIH-Knob) | | | IL-12p40-Fc (LALADS-KIH-Hole) | |
|---|---|---|---|---|---|---|
| | | | | SEQ ID | | SEQ ID |
| IL-12 Analog | Name | IL-12p35 subunit | | NO: | IL-12p40 subunit | NO: |
| IL-12-Fc-1 | 14BKP | p35 | E40K | 3 | WT | 2 |
| IL-12-Fc-2 | 15BKP | p35 | E40R | 4 | WT | 2 |
| IL-12-Fc-3 | 16BKP | p35 | E40W | 5 | WT | 2 |
| IL-12-Fc-4 | 17BKP | p35 | I41A | 6 | WT | 2 |
| IL-12-Fc-5 | 18BKP | p35 | I41V | 7 | WT | 2 |
| IL-12-Fc-6 | 19BKP | p35 | I41S | 8 | WT | 2 |
| IL-12-Fc-7 | 20BKP | p35 | I41T | 9 | WT | 2 |
| IL-12-Fc-8 | 21BKP | p35 | D42A | 10 | WT | 2 |
| IL-12-Fc-9 | 22BKP | p35 | D42L | 11 | WT | 2 |
| IL-12-Fc-10 | 23BKP | p35 | D42W | 12 | WT | 2 |
| IL-12-Fc-11 | 24BKP | p35 | E44A | 13 | WT | 2 |
| IL-12-Fc-12 | 25BKP | p35 | E44K | 14 | WT | 2 |
| IL-12-Fc-13 | 26BKP | p35 | E44R | 15 | WT | 2 |
| IL-12-Fc-14 | 27BKP | p35 | D159K | 16 | WT | 2 |
| IL-12-Fc-15 | 28BKP | p35 | D159R | 17 | WT | 2 |
| IL-12-Fc-16 | 29BKP | p35 | D159Y | 18 | WT | 2 |
| IL-12-Fc-17 | 30BKP | p35 | D159F | 19 | WT | 2 |
| IL-12-Fc-18 | 31BKP | p35 | Y161S | 20 | WT | 2 |
| IL-12-Fc-19 | 32BKP | p35 | Y161T | 21 | WT | 2 |
| IL-12-Fc-20 | 33BKP | p35 | Y161A | 22 | WT | 2 |
| IL-12-Fc-21 | 34BKP | p35 | Y161W | 23 | WT | 2 |
| IL-12-Fc-22 | 35BKP | p35 | K162E | 24 | WT | 2 |
| IL-12-Fc-23 | 36BKP | p35 | K162D | 25 | WT | 2 |

WT = IL-12p40 wild-type amino acid sequence (SEQ ID NO: 134)
IL-12p35 is linked to N-terminus of Fc via (GGGGS)₃ peptide linker (SEQ ID NO: 206)
IL-12p40 is linked to N-terminus of Fc via a 4G peptide linker (SEQ ID NO: 202)
IL-12p35 subunits have N-terminal hexapeptide truncation

TABLE 50

| | | p35-Fc (LALADS-KIH-Knob) | | p40-Fc (LALADS-KIH-Hole) | | |
|---|---|---|---|---|---|---|
| | | | SEQ ID | | | SEQ |
| IL-12 Analog | Name | p35 Subunit | NO: | p40 Subunit | | ID NO: |
| IL-12-Fc-24 | 37BKP | WT | 1 | p40 | K85E | 26 |
| IL-12-Fc-25 | 38BKP | WT | 1 | p40 | K85Y | 27 |
| IL-12-Fc-26 | 39BKP | WT | 1 | p40 | D87A | 28 |
| IL-12-Fc-27 | 40BKP | WT | 1 | p40 | D87L | 29 |
| IL-12-Fc-28 | 41BKP | WT | 1 | p40 | I89A | 30 |
| IL-12-Fc-29 | 42BKP | WT | 1 | p40 | I89S | 31 |
| IL-12-Fc-30 | 43BKP | WT | 1 | p40 | I89D | 32 |
| IL-12-Fc-31 | 44BKP | WT | 1 | p40 | I89P | 33 |
| IL-12-Fc-32 | 45BKP | WT | 1 | p40 | K104E | 34 |
| IL-12-Fc-33 | 46BKP | WT | 1 | p40 | K104D | 35 |
| IL-12-Fc-34 | 47BKP | WT | 1 | p40 | K104W | 36 |
| IL-12-Fc-35 | 48BKP | WT | 1 | p40 | K104S | 37 |

WT = IL-12p35 wild-type amino acid sequence with N-terminal hexapeptide truncation
IL-12p35 is linked to N-terminus of Fc via (GGGGS)₃ peptide linker (SEQ ID NO: 206)
IL-12p40 is linked to N-terminus of Fc via a 4G peptide linker (SEQ ID NO: 202)

TABLE 51

| | | p35-Fc (LALADS-KIH-Knob) | | | p40-Fc (LALADS- KIH-Hole) | | |
|---|---|---|---|---|---|---|---|
| | | | | SEQ | | | SEQ |
| IL-12-Analog | Name | p35 Subunit | | ID NO: | p40 Subunit | | ID NO: |
| IL-12-Fc-36 | 35 | p35 | I41S | 8 | p40 | D87L | 29 |
| IL-12-Fc-37 | 36 | p35 | E44R | 15 | p40 | D87L | 29 |
| IL-12-Fc-38 | 37 | p35 | D159K | 16 | p40 | D87L | 29 |
| IL-12-Fc-39 | 38 | p35 | D159R | 17 | p40 | D87L | 29 |
| IL-12-Fc-40 | 39 | p35 | Y161S | 20 | p40 | D87L | 29 |
| IL-12-Fc-41 | 40 | p35 | Y161T | 21 | p40 | D87L | 29 |
| IL-12-Fc-42 | 41 | p35 | Y161A | 22 | p40 | D87L | 29 |
| IL-12-Fc-43 | 42 | p35 | I41S | 8 | p40 | K104E | 34 |
| IL-12-Fc-44 | 43 | p35 | E44R | 15 | p40 | K104E | 34 |
| IL-12-Fc-45 | 44 | p35 | D159K | 16 | p40 | K104E | 34 |
| IL-12-Fc-46 | 45 | p35 | D159R | 17 | p40 | K104E | 34 |
| IL-12-Fc-47 | 46 | p35 | Y161S | 20 | p40 | K104E | 34 |
| IL-12-Fc-48 | 47 | p35 | Y161T | 21 | p40 | K104E | 34 |
| IL-12-Fc-49 | 48 | p35 | Y161A | 22 | p40 | K104E | 34 |
| IL-12-Fc-50 | 49 | p35 | I41S | 8 | p40 | K104D | 35 |
| IL-12-Fc-51 | 50 | p35 | E44R | 15 | p40 | K104D | 35 |
| IL-12-Fc-52 | 51 | p35 | D159K | 16 | p40 | K104D | 35 |
| IL-12-Fc-53 | 52 | p35 | D159R | 17 | p40 | K104D | 35 |
| IL-12-Fc-54 | 53 | p35 | Y161S | 20 | p40 | K104D | 35 |
| IL-12-Fc-55 | 54 | p35 | Y161T | 21 | p40 | K104D | 35 |
| IL-12-Fc-56 | 55 | p35 | Y161A | 22 | p40 | K104D | 35 |

IL-12p35 is linked to N-terminus of Fc via (GGGGS)₃ peptide linker (SEQ ID NO: 206)
IL-12p40 is linked to N-terminus of Fc via a 4G peptide linker (SEQ ID NO: 202)
IL-12p35 has with N-terminal hexapeptide truncation

Assays using these cells were performed according to the manufacturer's protocol.

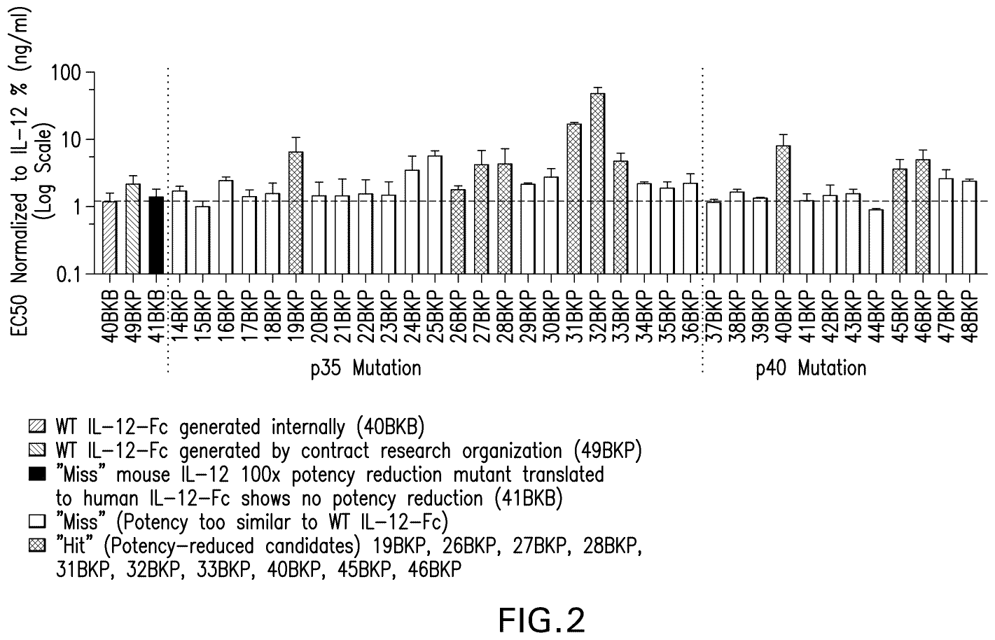

Briefly, HEK-Blue™ IL-12 cells were cultured overnight with different concentrations of monovalent IL-12 heterodimeric Fc protein comprising various IL-12p35 and/or IL-12p40 subunit mutations (IL-12 variants) or WT IL-12p35 and IL-12p40 subunits (IL-12 WT) in growth medium (Dulbecco Modified Eagles Medium (DMEM), 4.5 g/L glucose, 2 mM L-glutamine, 10% (v/v) heat-inactivated fetal bovine serum, 100 U/mL penicillin, 100 μg/mL streptomycin, 100 μg/mL Normocin™ (Invivogen). The next day, supernatant fractions were taken and mixed with QUANTI-Blue™ Solution to detect SLAP production for measurement of optical density using a spectrophotometer to determine the level of STAT4 induction. Non-linear curve fitting was performed on the spectrophotometer readings to calculate EC₅₀ values. FIG. 2 shows the potency of various monovalent IL-12 heterodimeric Fc proteins comprising a single amino acid substitution in either the L-12p35 or IL-12p40 subunit (see Table 49 and Table 50). 41BKP comprises amino acid substitutions in the IL-12p35 subunit, which when present in the mouse IL-12p35 subunit showed a 100-fold reduction in potency, but as shown in FIG. 2 the same amino acid substitutions in the monovalent IL-12 heterodimeric Fc protein did not result in a reduction potency. Mouse IL-12 mutants were based on Zou et al., JBC, 1995, Vol. 270, No 11, pp. 5864-5871). The potency data are also presented in Table 52 and Table 53.

TABLE 52

| | | | | Mean |
|---|---|---|---|---|
| | | IL-12p35 | IL-12p40 | Fold |
| IL-12 Analog | Name | subunit | subunit | Reduction |
| IL-12-Fc-wt1 | 40BKB | WT | WT^{a} | 1 |
| mIL-12-Fc-wt | 41BKB | mWT | mWT^{a} | 1 |
| IL-12-Fc-1 | 14BKP | p35 E40K | WT^{a} | 2 |
| IL-12-Fc-2 | 15BKP | p35 E40R | WT^{a} | 1 |
| IL-12-Fc-3 | 16BKP | p35 E40W | WT^{a} | 2 |
| IL-12-Fc-4 | 17BKP | p35 I41A | WT^{a} | 1 |
| IL-12-Fc-5 | 18BKP | p35 I41V | WT^{a} | 2 |
| IL-12-Fc-6 | 19BKP | p35 I41S | WT^{a} | 7 |
| IL-12-Fc-7 | 20BKP | p35 I41T | WT^{a} | 1 |
| IL-12-Fc-8 | 21BKP | p35 D42A | WT^{a} | 1 |
| IL-12-Fc-9 | 22BKP | p35 D42L | WT^{a} | 2 |
| IL-12-Fc-10 | 23BKP | p35 D42W | WT^{a} | 1 |
| IL-12-Fc-11 | 24BKP | p35 E44A | WT^{a} | 4 |
| IL-12-Fc-12 | 25BKP | p35 E44K | WT^{a} | 6 |
| IL-12-Fc-13 | 26BKP | p35 E44R | WT^{a} | 2 |
| IL-12-Fc-14 | 27BKP | p35 D159K | WT^{a} | 4 |
| IL-12-Fc-15 | 28BKP | p35 D159R | WT^{a} | 4 |
| IL-12-Fc-16 | 29BKP | p35 D159Y | WT^{a} | 2 |
| IL-12-Fc-17 | 30BKP | p35 D159F | WT^{a} | 3 |
| IL-12-Fc-18 | 31BKP | p35 Y161S | WT^{a} | 17 |
| IL-12-Fc-19 | 32BKP | p35 Y161T | WT^{a} | 49 |
| IL-12-Fc-20 | 33BKP | p35 Y161A | WT^{a} | 5 |
| IL-12-Fc-21 | 34BKP | p35 Y161W | WT^{a} | 2 |
| IL-12-Fc-22 | 35BKP | p35 K162E | WT^{a} | 2 |
| IL-12-Fc-23 | 36BKP | p35 K162D | WT^{a} | 1 |
| IL-12-Fc-24 | 37BKP | WT | p40 K85E | 1 |
| IL-12-Fc-25 | 38BKP | WT | p40 K85Y | 2 |
| IL-12-Fc-26 | 39BKP | WT | p40 D87A | 1 |
| IL-12-Fc-27 | 40BKP | WT | p40 D87L | 8 |
| IL-12-Fc-28 | 41BKP | WT | p40 I89A | 1 |
| IL-12-Fc-29 | 42BKP | WT | p40 I89S | 1 |
| IL-12-Fc-30 | 43BKP | WT | p40 I89D | 2 |
| IL-12-Fc-31 | 44BKP | WT | p40 I89P | 1 |
| IL-12-Fc-32 | 45BKP | WT | p40 K104E | 4 |
| IL-12-Fc-33 | 46BKP | WT | p40 K104D | 5 |
| IL-12-Fc-34 | 47BKP | WT | p40 K104W | 3 |
| IL-12-Fc-35 | 48BKP | WT | p40 K104S | 2 |
| IL-12-Fc-wt2 | 49BKP | WT | WT^{a} | 2 |

WT = wild-type IL-12p35 amino acid sequence with N-terminal hexapeptide truncation (SEQ ID NO: 133)
WT^{a} = IL-12p40 wild-type amino acid sequence (SEQ ID NO: 134)
mWT = wild-type IL-12p35 with deletion of N-terminal hexapeptide and comprising K, H, Y, S, H, and P amino acid substitutions at positions 32, 33, 34, 35, 122, and 160, respectively
IL-12p35 is linked to N-terminus of Fc via (GGGGS)3 peptide linker (SEQ ID NO: 206)
IL-12p40 is liked to N-terminus of Fc via a 4G peptide linker (SEQ ID NO: 202)

TABLE 53

| | | | | Mean |
|---|---|---|---|---|
| | | | | Fold |
| IL-12-Analog | Name | p35 Subunit | p40 Subunit | Reduction |
| IL-12-Fc-wt1 | 40BKB | WT | WT^{a} | 1 |
| IL-12-Fc-36 | 35 | p35 141S | p40 D87L | 1 |
| IL-12-Fc-37 | 36 | p35 E44R | p40 D87L | 1 |
| IL-12-Fc-38 | 37 | p35 D159K | p40 D87L | 1 |
| IL-12-Fc-39 | 38 | p35 D159R | p40 D87L | 2 |
| IL-12-Fc-40 | 39 | p35 Y161S | p40 D87L | 12 |
| IL-12-Fc-41 | 40 | p35 Y161T | p40 D87L | 80 |
| IL-12-Fc-42 | 41 | p35 Y161A | p40 D87L | 5 |
| IL-12-Fc-43 | 42 | p35 141S | p40 K104E | 3 |
| IL-12-Fc-44 | 43 | p35 E44R | p40 K104E | 3 |
| IL-12-Fc-45 | 44 | p35 D159K | p40 K104E | 1 |
| IL-12-Fc-46 | 45 | p35 D159R | p40 K104E | 1 |
| IL-12-Fc-47 | 46 | p35 Y161S | p40 K104E | 45 |
| IL-12-Fc-48 | 47 | p35 Y161T | p40 K104E | 209 |
| IL-12-Fc-49 | 48 | p35 Y161A | p40 K104E | 6 |
| IL-12-Fc-50 | 49 | p35 141S | p40 K104D | 2 |
| IL-12-Fc-51 | 50 | p35 E44R | p40 K104D | 2 |
| IL-12-Fc-52 | 51 | p35 D159K | p40 K104D | 2 |
| IL-12-Fc-53 | 52 | p35 D159R | p40 K104D | 2 |
| IL-12-Fc-54 | 53 | p35 Y161S | p40 K104D | 195 |
| IL-12-Fc-55 | 54 | p35 Y161T | p40 K104D | 2 |
| IL-12-Fc-56 | 55 | p35 Y161A | p40 K104D | 32 |

WT = IL-12p35 wild-type amino acid sequence with deletion of N-terminal hexapeptide (SEQ ID NO: 133)
WT^{a} = IL-12p40 wild-type amino acid sequence (SEQ ID NO: 134)
IL-12p35 is linked to N-terminus of Fc via (GGGGS)3 peptide linker (SEQ ID NO: 206)
IL-12p40 is linked to N-terminus of Fc via a 4G peptide linker (SEQ ID NO: 202)

IL-12 variants with greater than 5-fold reduction vs. IL-12 WT potency (as determined using average EC₅₀ values of two-to-three independent experiments) were selected as the basis for combinational mutagenesis in additional arounds of engineering. IL-12 variants with roughly 10, 100, 1,000, or 10,000 fold reduction in potency (vs IL-12 WT; through EC₅₀ value comparison) were selected for additional characterization in primary human or Non-Human Primate (NHP) blood cells. The greatest reduction in potency was observed for 19BKP, 31BKP, 32BKP, 33BKP, 40BKP, 45BKP, and 46BKP.

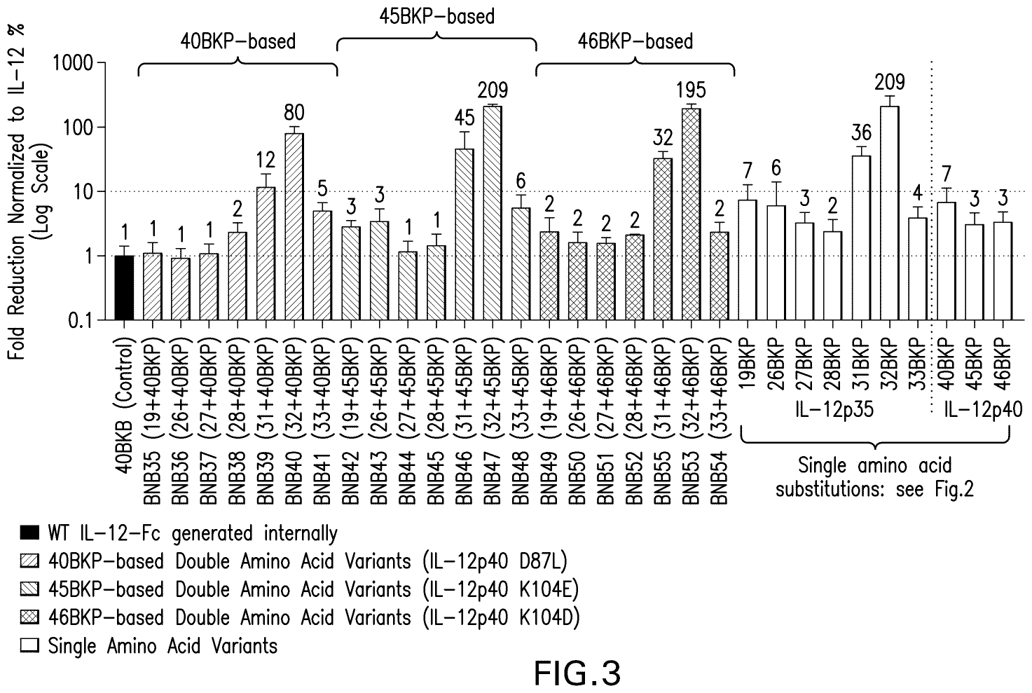

FIG. 3 shows the potency of various monovalent IL-12 heterodimeric Fc proteins comprising a single amino acid substitution in the IL-12p35 and in the IL-12p40 subunit (See Table 51). The results show that there was no synergistic potency reduction observed with the double mutants compared to the single mutants.

FIG. 4 shows that 31BKP and 32BKP showed the greatest reduction in potency, 36% and 209%, respectively.

### Example 2

Confirming In Vitro Potency of Monovalent IL-12 Heterodimeric Fc Proteins with IFNγ Release Assays in Primary Human and Non-Human Primate (NHP) Blood Cells.

Human and NHP peripheral blood mononuclear cell (PBMC) isolation: Human or NHP PBMCs were obtained by overlaying whole blood or leukopak over Percoll-based separation media (different formulation was used for human and NHP) and spun through centrifugation. Isolated cells were then extensively washed and residual red blood cells (RBC) were lysed through RBC lysis buffer. Resulting PBMCs were then frozen and kept in liquid nitrogen until use.

In vitro IFNγ release from activated primary PBMCs: In a 96-well U-bottom plate, 100K-200K PBMCs were co-stimulated with phytohemagglutinin-L (PHA-L) in RPMI media and various concentrations of IL-12 variants. Supernatant fractions were harvested after 72 hours and IFNγ concentration determined according to manufacturer protocol (Mesoscale Discovery: catalog code #K151TTK-2). Non-linear curve fitting was performed on the IFNγ concentration to calculate EC₅₀ values. EC₅₀ values of an IL-12 variant, as determined in reporter gene activity in HEK-Blue™ IL-12 cells, IFNγ release from primary human PBMC, and IFNγ release from NHP PBMC, that differed by less than 10-fold between the three assays were selected for additional in vitro and in vivo characterization. Table 52 shows reporter gene activity in HEK-Blue™ IL-12 cells, IFNγ release from primary human PBMC, and IFNγ release from NHP PBMC for several representative samples.

Target engagement is determined by reporter activity (in vitro) and IFNγ release (in vitro and in vivo). The results are reported in Table 54.

TABLE 54

| | HEK Reporter | Human PBMC | Rhesus PBMC |
|---|---|---|---|
| Monovalent IL-12 | Median EC₅₀ | Median EC₅₀ | Median EC₅₀ |
| heterodimeric Fc protein | values (ng/mL) | values (ng/ml) | values (ng/ml) |
| IL-12-Fc-wt2 (49BKP) | 1.4 | 1.4 | 0.3 |
| IL-12-Fc-18 (31BKP) (30-fold) | 35 | 33 | 87 |
| IL-12-Fc-19 (32BKP) (200-fold) | 198 | 178 | 365 |
| IL-12-Fc-41 (4,000-fold) | 3641 | 2852 | N.D. |
| IL-12-Fc-60 (10,000-fold) | 11,662 | N.D. | N.D. |

Median EC₅₀ values determined via HEK reporter cell line (n = 3), IFNγ release fromactivated human PBMC in vitro (n = 8), and IFNγ release from activated rhesus PBMC in vitro (n = 10). N.D. = not determined

### Example 3

### Dose Dependent Reduction of Tumor Size by IL-12-Fc-19 (200× Potency Reduced IL-12-Fc) and IL-12-Fc-259 (30× Potency Reduced IL-12-Fc-YTE) in Human PBMC Transferred PANC08 Xenografted Mouse Model.

To determine in vivo efficacy of the potency reduced IL-12 mutants, we used “humanized” mouse model where immunodeficient NSG-MHC I/II double knockout mice (Jackson Laboratory Strain #025216) were reconstituted with human PBMCs. As human IL-12 does not cross react with mouse, the humanized model was required to examine IL-12 mutants in a xenograft tumor setting. The model also allows analysis of changes in human immune cells in vivo. Mice were inoculated subcutaneously (s.c.) in the right rear flank with 1×10⁶ Panc 08.13 human pancreatic cancer cell line. When tumors were palpable (day 7), mice were engrafted with 10×10⁶ healthy donor human PBMCs. Seven days after PBMC reconstitution, mice received weekly injections of potency reduced IL-12 mutants or isotype control antibodies for a total of 4 weeks. PBMC engrafted mice did not display symptoms of graft-vs.-host disease or symptoms of autoimmunity during the course of the experiments. Body weight and tumor volume measurements were taken twice a week during the study. Tumor length and width were measured using electronic calipers (and tumor volumes were determined using the following formula: Volume (mm³)=0.5×length×width²; where length is the longer dimension.

In addition to efficacy, we also wanted to correlate drug exposure with PD response and tumor efficacy. For the IL-12-Fc-19 only, there are two arms for each treatment group: “Efficacy arm” and “PK/PD arm”. In Efficacy arm: Dose dependent reduction of tumor size was observed after day 10. Two mice in the high dose group euthanized due to body weight loss more than 20%. In PK/PD arm the objective was to obtain samples for drug exposure and PD measurements before and after significant tumor size changes in response to treatment. The results demonstrated a consistent PK/PD relationship.

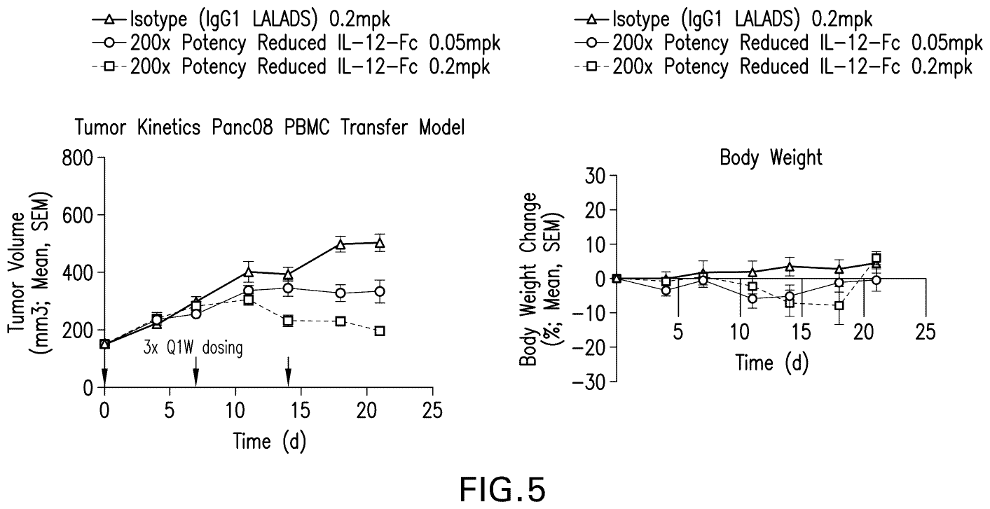

FIG. 5 shows in the PANC08 Xenograph Mouse Model that dosing with IL-12-Fc-19 reduced tumor volume over time with no significant change in body weight.

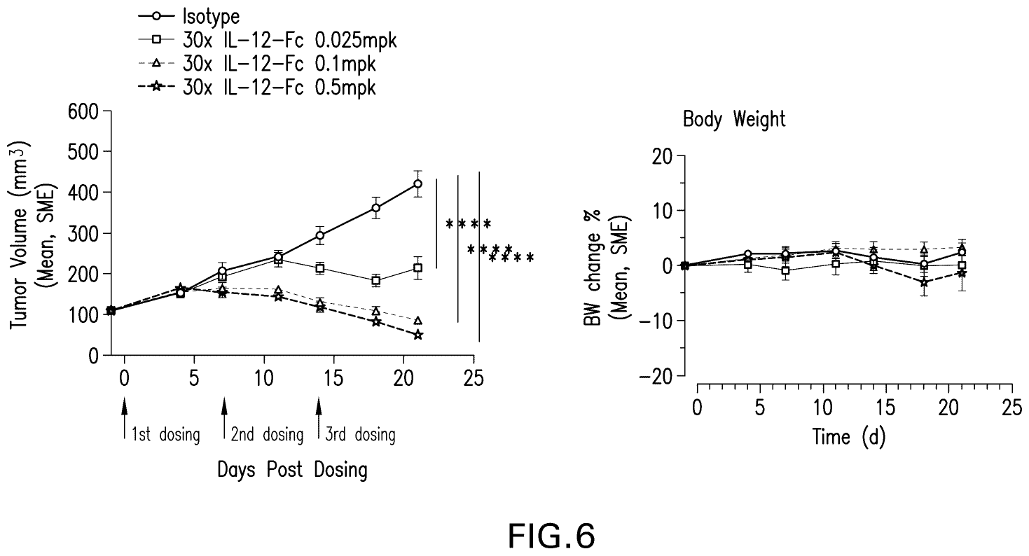

FIG. 6 shows in the PANC08 Xenograph Mouse Model that dosing with IL-12-Fc-259 reduced tumor volume over time with no significant change in body weight. The PANC08 Xenograph Mouse Model did not demonstrate any significant difference between IL-12-Fc-259 and IL-12-Fc-19.

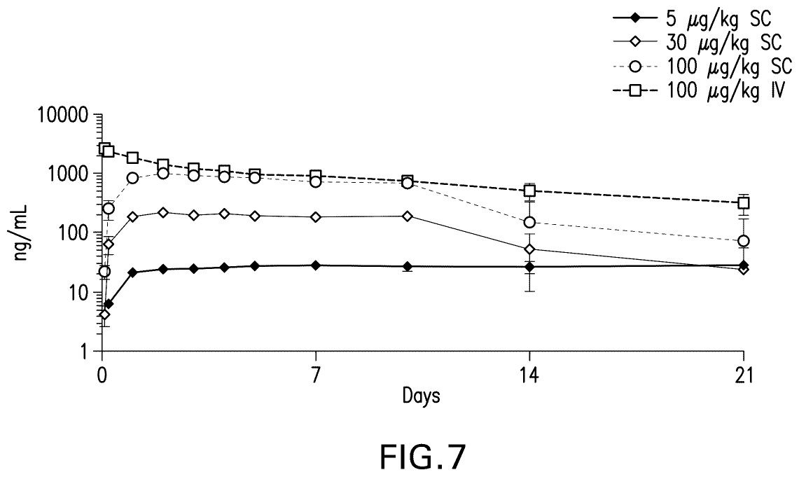

FIG. 7 shows prolonged PK exposure of IL-12-Fc-259 greater than 13 days and while maintaining a sustained pharmacodynamic (PD) response in non-human primates (NHP). The PD response was drive by exposure. Overall, the example shows that extended half-life and sustained IFNγ are not expected with current clinical competitors.

### Example 4

Pharmacodynamic/Pharmacokinetic Relationship in NHP Showed Longer Drug Exposure that Translates in a More Sustained PD Response.

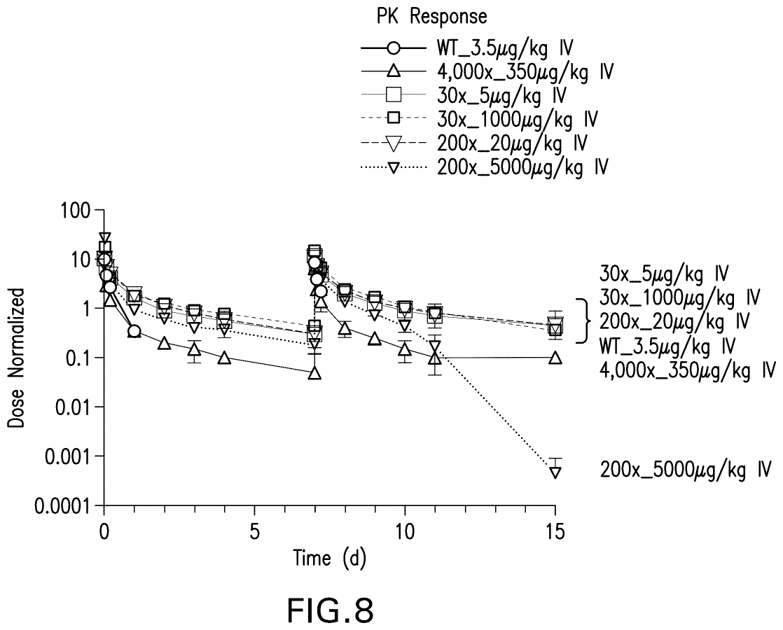

For this Example, NHP were dosed with IL-12-Fc-18 (30× reduced potency), IL-12-Fc-19 (200× reduced potency), and AL-12-Fc-58 (4,000× reduced potency) compared to wild-type L-12-Fc (WI) for Q1W×2 Dosing in Rhesus Monkeys. Then the doses were increased 200× for L-12-Fc-18 (30× reduced potency), L-12-Fc-19 (200× reduced potency) and animals were dosed twice on Day 0 and Day 7. FIG. 8 shows the pharmacokinetic response over time for IL-12-Fc-18 (30× reduced potency), IL-12-Fc-19 (200× reduced potency), and L-12-Fc-58 (4,00× reduced potency) compared to wild-type L-12-Fc (WI) for Q1W×2 Dosing in Rhesus Monkeys. L-12-Fc-18 and IL-12-19 showed significant gain in exposure relative to wild-type IL-12-Fc and L-12-Fc-58. The L-12-Fc-19 loss of exposure in the higher dose level likely due to presence of anti-drug antibody (ADA). In vivo stability was confirmed by total vs. intact assay measurements. The results are summarized in Table 55.

TABLE 55
NCA obtained PK parameters after each dose in rhesus monkeys

| Treatment | # | AUClast | AUC/ | CL | MRT |
|---|---|---|---|---|---|
| STEP-1 | Doses | (day*ng/mL) | Dose | (mL/day/kg) | (d) |
| WT IL-12-Fc | 1 | 8 | 0.0020 | 409 | 0.2 |
| | 2 | 3 | 0.0008 | 812 | 0.07 |
| IL-12-Fc-18 | 1 | 38 | 0.0080 | 112 | 1.7 |
| (30x) | 2 | 48 | 0.0090 | 83 | 1.9 |
| IL-12-Fc-19 | 1 | 181 | 0.0090 | 95 | 1.7 |
| (200x) | 2 | 203 | 0.0100 | 80 | 1.9 |
| IL-12-Fc-58 | 1 | 792 | 0.0020 | 401 | 1.1 |
| (4,000x) | 2 | 802 | 0.0020 | 373 | 1.4 |
| Treatment | # | AUClast | AUC/ | CL | MRT |
| STEP-2 | Doses | (day*ng/mL) | Dose | (mL/day/kg) | (d) |
| IL-12-Fc-18 | 1 | 10024 | 0.017 | 81 | 1.8 |
| (30x) | 2 | 11970 | 0.014 | 75 | 1.7 |
| IL-12-Fc-19 | 1 | 32408 | 0.025 | 137 | 1.3 |
| (200x) | 2 | 28192 | 0.006 | 171 | 0.9 |

CL = clearance
AUC = area under the curve
MRT = mean residence time
30x, 200x, 4,000x = fold reduction in potency

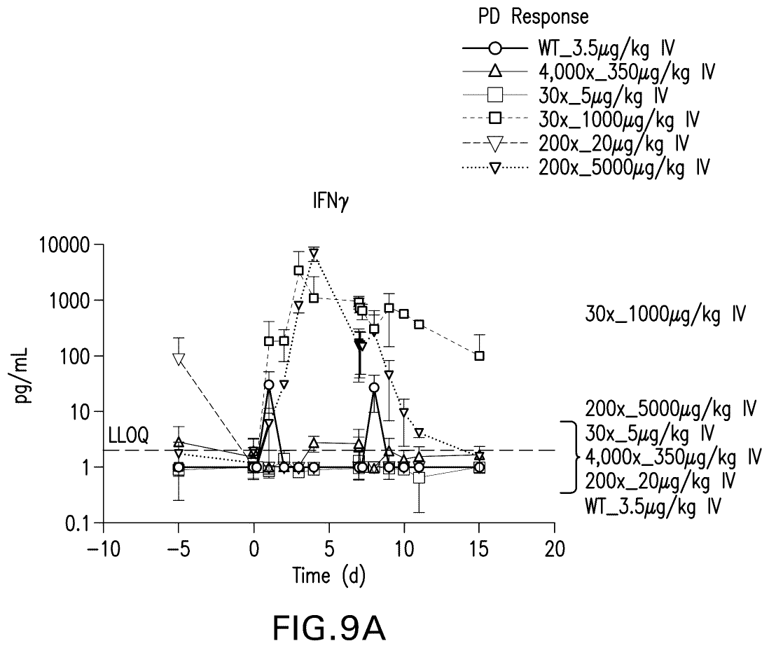

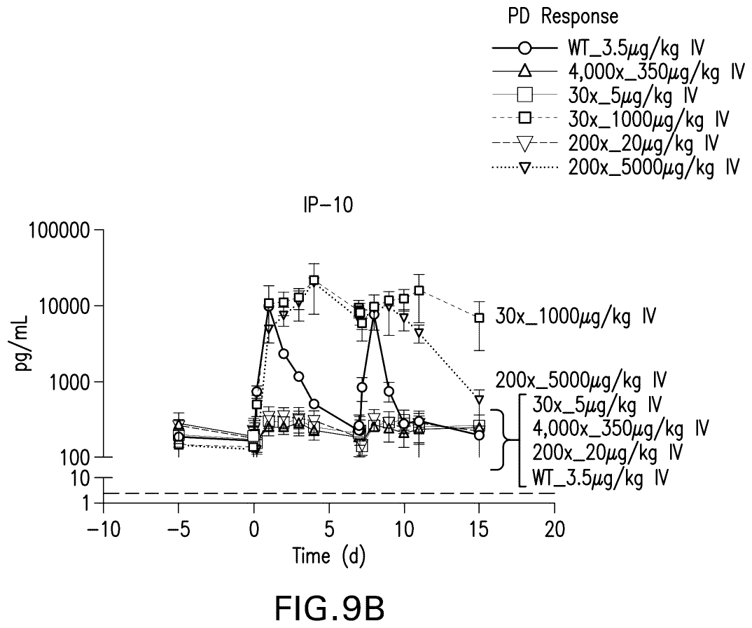

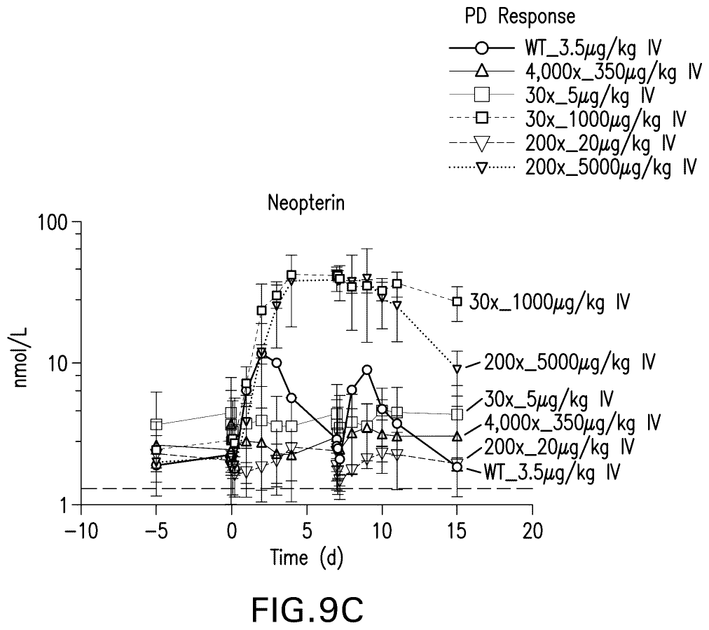

FIG. 9A, FIG. 9B, and FIG. 9C show the pharmacodynamic (PD) response over time for IL-12-Fc-18 (30× reduced potency), IL-12-Fc-19 (200× reduced potency), and IL-12-Fc-58 (4,000× reduced potency) compared to wild-type IL-12-Fc (WT) for QIW×2 Dosing in Rhesus Monkeys. The figures show that the 30× and 200× potency reduced IL-12-Fc sustained PD response was maintained with Q1W Dose Regimen. The PD response only observed with wild-type IL-12-Fc in Step 1 which was dosed at 200× EC₅₀ at Cₘₐₓ (others were dosed at EC₅₀)-Consistent PK/PD relationship was observed across the 30× and 200× muteins in Step 2. In addition to cytokines, the 30× and 200× muteins in Step 2 induced a sustained increase in CD64+monocytes (CD64 expression on monocyte can be induced by IFNγ). In Step 2, both the 30× and 200× muteins were well tolerated—only transient decreases in lymphocytes/monocytes (4 animals) and transient increase in serum CRP level (1 in each group; total 2 out of 4 animals) were attributed to treatment.

### Example 5

### The Effect of N-Glycan Sialylation on Potency and Exposure was Evaluated for IL-12-Fc-259. Generation of 15%, 20% and 25% N-Glycan Sialylation Material

A research cell bank of the stable transfected pools expressing IL-12-FC-259 (p35 Y161S-15GS-hIgG1 Fc LALADS YTE KIH knob/p40-4G-IgG1 Fc LALADS YTE KIH Hole) in CHO-KI glutamine synthetase knockout (GSKO) cells was thawed in Selection Media (GIBCO CD CHO medium (Thermo Fisher Scientific, Waltham, MA)+1×GIBCO HT 100× supplement (Thermo Fisher Scientific)+12.5 μM MSX (methionine sulfoxamine). The cells were passaged at a seeding density of 0.3-0.5×10⁶ viable cells/mL every 2-3 days for 10 passages in 125 mL-250 mL Optimum Growth Shake Flasks in Selection Media. The cells from passage 10 (N-2 seed train) were used to inoculate N−1 culture in fresh Fed Batch Production Medium (GIBCO DYNAMIS medium+1×GIBCO HT 100× supplement+1 μM CuSO₄) in an appropriate volume to target a seeding density of 0.5×10⁶ viable cells/mL to inoculate production culture.

To generate material with different sialylation levels, cells from N−1 culture were seeded into 750 mL of fresh Fed Batch Production Medium (DYNAMIS medium+1×GIBCO HT 100× supplement+1 μM CuSO₄) in a 1.6 L Optimum Growth Shake Flask (Thomson Instrument Company, Oceanside CA) at two different seeding density and supplemented with different feeds and feeding regime as follows:
a) Seeded in duplicate at 2.0×10⁶ viable cells/mL supplemented with HYCLONE CELL BOOST commercial feeds 7a and 7b (Cytiva Life Sciences, Marlborough, MA) day 2 onwards, into fed batch production culture at 2.7% v/v and 0.30% v/v, respectively
b) Seeded in duplicate at 0.5×10⁶ viable cells/mL supplemented with chemically defined in-house feeds on days 3, 5, 7, 9 and 11, into fed batch production culture at 5.12% v/v and 0.60% v/v, respectively.

On Day 0, 0.5 μM of MnSO₄ was added to the fed batch production culture for both the fed batch production cultures. Additionally, D-glucose was added daily starting from day 2 to maintain the culture glucose level at 6-8 g/L. All cells were incubated in a shaking incubator at 36.5° C., 5% CO₂, 80% humidity, 140 revolutions per minute (RPM) and 2″ orbital diameter.

Cells from fed batch production culture fed with commercial feeds were harvested on day 14 and day 17 at harvest viability of 74% and 70%, respectively, yielded protein compositions having a sialic acid content of 12.28% and 11.45%, respectively, as measured by liquid chromatography (LC) coupled to fluorescent detection (FLR)N-glycan analysis method. Similarly, cells from fed batch production culture fed with in-house feeds were harvested on day 12 at harvest viability of 90.1% and 91.1%, yielding protein with a sialic acid content of 30.25% and 29.56%, respectively, as measured by LC-FLR N-glycan analysis method.

These materials with different sialic acid contents were then mixed in molar fraction to generate IL-12-Fc-259 composition groups with 3 different levels of N-glycan sialylation (15%, 20% and 25%). Table 56 below lists the major N-glycan species for the compositions of the three groups as analyzed by LC-FLR method.

TABLE 56

| N-Glycan Species | Group 1 | Group 2 | Group 3 |
|---|---|---|---|
| High mannose N-glycans | 32.21% | 33.13% | 34.72% |
| Afucosylated N-glycans | 1.49% | 1.68% | 1.95% |
| Galactose-terminated N-glycans | 46.48% | 42.17% | 37.34% |
| Sialic Acid-terminated N-glycans | 24.30% | 20.33% | 15.72% |

Mouse PK PD studies using TG32 mice (cRn−/− hFcRn (32) Tg, hFcRn Tg32 from Jackson Laboratory, Bar harbor, ME), were carried out using the IL-12-Fc-259 from the three groups to understand the impact of sialylation on clearance and potency. TG32 mice were dosed with compositions of IL-12-Fc-259 with the levels of sialylation as shown in Table 57 below.

TABLE 57

| | Route of | Dose | % | # | Duration |
|---|---|---|---|---|---|
| Group | Administration | (mg/kg) | Sialylation | animals | (Days) |
| 1 | IV | 5 | 15 | 6 | 14 |
| 2 | IV | 5 | 20 | 6 | 14 |
| 3 | IV | 5 | 25 | 6 | 14 |
| 4 | IV | 5 | 14.5 | 6 | 14 |

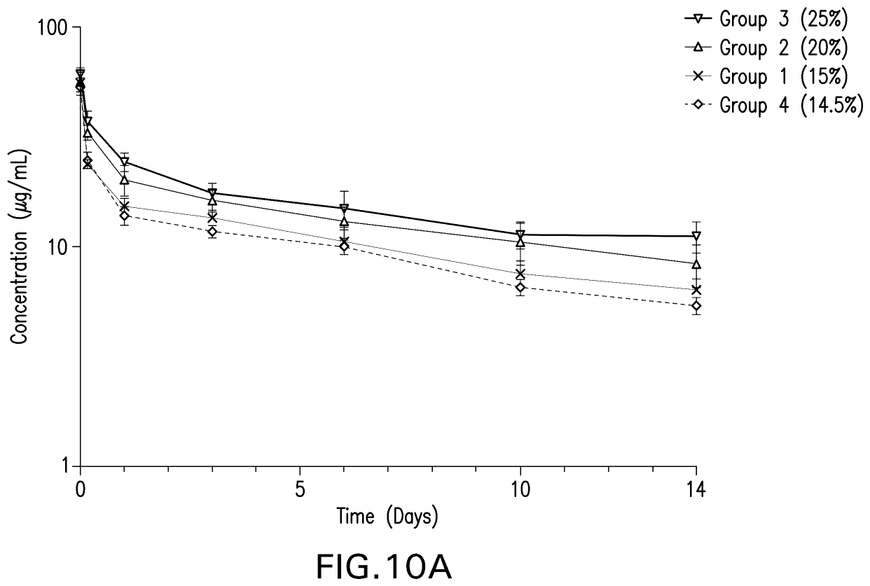

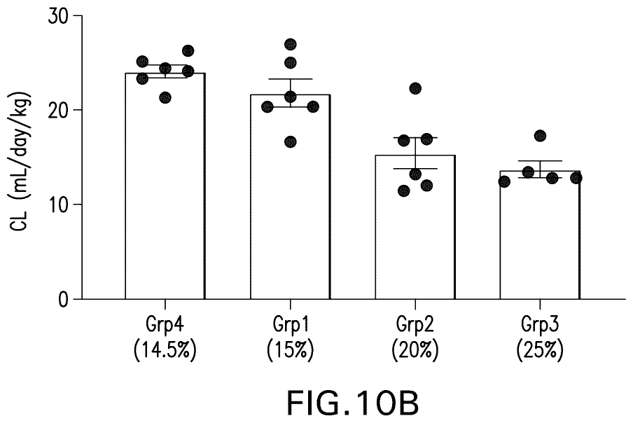

The study results are shown in FIG. 10A and FIG. 10B. FIG. 10A shows that the average serum concentration of 20-25% sialylated IL-12-Fc-259 after 14 days for each group of six mice was higher than that of 14.5-15% sialylated IL-12-Fc-259. FIG. 10B shows that the rate of clearance (CL) was reduced as the degree of sialylation was increased from 14.5-15% to 20-25%. The results are also summarized in Table 58.

TABLE 58

| | % | t_{1/2} | AUC 0-∞ | CL |
|---|---|---|---|---|
| Group | Sialyation | (day) | (day*∥g/mL) | (mL/day/kg) |
| 1 | 15% | 9.4 | 232 | 21.6 |
| 2 | 20% | 11.6 | 332 | 15.1 |
| 3 | 25% | 10.8 | 366 | 13.7 |
| 4 | 14.5% | 9.3 | 208 | 24.1 |

AUC = area under the curve
CL = clearance

The study results show that exposure/CL were statistically similar between Groups 2 and 3 and the change of exposure with sialylation leveled off after 20% sialylation. Thus, there was no significant difference in exposure between 20% and 25% sialylated material.

### LC-FLR Experimental Conditions:

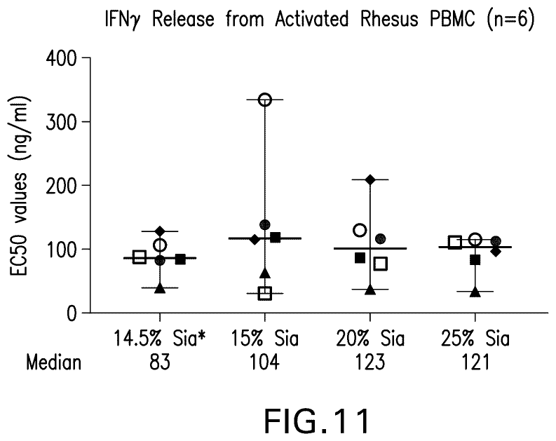

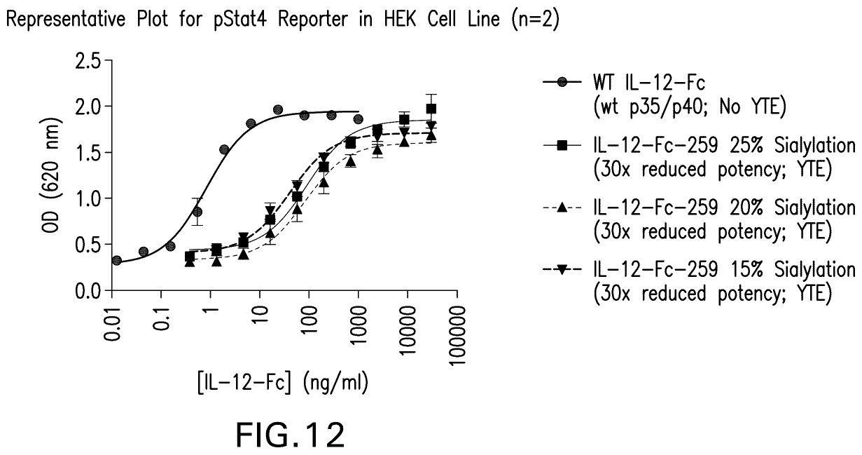

Samples were prepared following the iPC protocol. Following labeling, Samples were transferred to LC vials for LC-FLD-MS analysis and 1 μL was injected in the LC/MS system; 6230B TOF MS conditions: Agilent Jet Stream ESI source, any MS, positive mode, sheath gas 325° C. at 8 L/min, dry gas 150° C. at 9.0 L/min, nebulizer pressure 35 psig (pounds per square inch gauge), VCap 3500 V, Nozzle 1000 V, Fragmentor 175 V (if applicable), m/z range 100-3000; Column: 60-Minute UHPLC methods for Waters BEH Glycan Separation Technology column: 2.1×150 mm, 1.7 m column temperature 60° C., excitation 285 nm, emission 345 nm; Flow Rate: 0.4 mL/minute; Column Temperature: 60° C.; Buffer A: 50 mM Ammonium Formate, pH 4.4; Buffer B: CAN; Gradient Table: Run Time: 60 minutes; MS range: 100-3000 m/z. FIG. 11 and FIG. 12 show that the amount of sialylation of N-glycans on the p35 and p40 subunits did not impact potency of the 11-12 Fc analogs as exemplified by contacting activated Rhesus PBMCs with IL-12-Fc-259 using the protocol disclosed in Example 2. A one-way ANOVA with Tukey's test showed no significance in all test articles for the rhesus PBMC data. In FIG. 11, each symbol represents cells from one 1 donor and the bar shows the median ±95% Confidence Interval. FIG. 12 shows that sialic acid levels on attenuated IL-12-YTE-Fc did not impact potency in HEK cell line. WT IL-12-Fc consists of wt Il-12p35 linked to Fc consisting of LALADS-KIH-Knob) and wt IL-12p40 fused to Fc (LALADS-KIH-Hole).

Detailed N-glycan site occupancy data for IL-12-Fc-259 and IL-12-Fc-18 was also generated using MS quantitation showing relative levels of various N-glycan species at specific sites on the IL12-Fc molecules. MS quantitation was based on relative quant of summed peak areas from XIC (extracted ion chromatograms). Data attached in supplemental information.

### Example 6

The amino acid sequences and nucleotide sequences of the various IL-12 analogs and components thereof are presented in Table 59.

TABLE 59
Amino Acid and Nucleotide Sequences

| SEQ | | |
|---|---|---|
| ID | | |
| NO: | Description | Sequence |
| 1 | p35-15GS-Fc | TPDPGMFPCLHHSQNLLRAVSNMLQKARQTLEFYPCTSEEID |
| | (LALADS-KIH | HEDITKDKTSTVEACLPLELTKNESCLNSRETSFITNGSCLAS |
| | Knob: S354C- | RKTSFMMALCLSSIYEDLKMYQVEFKTMNAKLLMDPKRQIF |
| | T366W) | LDQNMLAVIDELMQALNFNSETVPQKSSLEEPDFYKTKIKLC |
| | | ILLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 2 | p40-4G-Fc | IWELKKDVYVVELDWYPDAPGEMVVLTCDTPEEDGITWTL |
| | (LALADS-KIH | DQSSEVLGSGKTLTIQVKEFGDAGQYTCHKGGEVLSHSLLLL |
| | Hole: Y349C- | HKKEDGIWSTDILKDQKEPKNKTFLRCEAKNYSGRFTCWWL |
| | T366S-L368A- | TTISTDLTFSVKSSRGSSDPQGVTCGAATLSAERVRGDNKEY |
| | Y407V) | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLMISRTPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 3 | p35 (E40K)-15GS- | TPDPGMFPCLHHSQNLLRAVSNMLQKARQTLEFYPCTSEKID |
| | Fc (LALADS-KIH | HEDITKDKTSTVEACLPLELTKNESCLNSRETSFITNGSCLAS |
| | Knob: S354C- | RKTSFMMALCLSSIYEDLKMYQVEFKTMNAKLLMDPKRQIF |
| | T366W) | LDQNMLAVIDELMQALNFNSETVPQKSSLEEPDFYKTKIKLC |
| | | ILLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 4 | p35 (E40R)-15GS- | TPDPGMFPCLHHSQNLLRAVSNMLQKARQTLEFYPCTSERID |
| | Fc (LALADS-KIH | HEDITKDKTSTVEACLPLELTKNESCLNSRETSFITNGSCLAS |
| | Knob: S354C- | RKTSFMMALCLSSIYEDLKMYQVEFKTMNAKLLMDPKRQIF |
| | T366W) | LDQNMLAVIDELMQALNFNSETVPQKSSLEEPDFYKTKIKLC |
| | | ILLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 5 | p35 (E40W)- | TPDPGMFPCLHHSQNLLRAVSNMLQKARQTLEFYPCTSEWI |
| | 15GS-Fc | DHEDITKDKTSTVEACLPLELTKNESCLNSRETSFITNGSCLA |
| | (LALADS-KIH | SRKTSFMMALCLSSIYEDLKMYQVEFKTMNAKLLMDPKRQI |
| | Knob: S354C- | FLDQNMLAVIDELMQALNFNSETVPQKSSLEEPDFYKTKIKL |
| | T366W) | CILLHAFRIRAVTIDRVMSYLNASGGGGSGGGGSGGGGSD |
| | | KTHTCPPCPAPEAAGGPSVFLFPPKPKDTLMISRTPEVTCVVV |
| | | SVSHEDPEVKFNWYVDGVEVHNAKTKPREEQYNSTYRVVS |
| | | VLTVLHQDWLNGKEYKCKVSNKALPAPIEKTISKAKGQPRE |
| | | PQVYTLPPCRDELTKNQVSLWCLVKGFYPSDIAVEWESNGQ |
| | | PENNYKTTPPVLDSDGSFFLYSKLTVDKSRWQQGNVFSCSV |
| | | MHEALHNHYTQKSLSLSPGK |
| 6 | p35 (I41A)-15GS- | TPDPGMFPCLHHSQNLLRAVSNMLQKARQTLEFYPCTSEEA |
| | Fc (LALADS-KIH | DHEDITKDKTSTVEACLPLELTKNESCLNSRETSFITNGSCLA |
| | Knob: S354C- | SRKTSFMMALCLSSIYEDLKMYQVEFKTMNAKLLMDPKRQI |
| | T366W) | FLDQNMLAVIDELMQALNFNSETVPQKSSLEEPDFYKTKIKL |
| | | CILLHAFRIRAVTIDRVMSYLNASGGGGSGGGGSGGGGSD |
| | | KTHTCPPCPAPEAAGGPSVFLFPPKPKDTLMISRTPEVTCVVV |
| | | SVSHEDPEVKFNWYVDGVEVHNAKTKPREEQYNSTYRVVS |
| | | VLTVLHQDWLNGKEYKCKVSNKALPAPIEKTISKAKGQPRE |
| | | PQVYTLPPCRDELTKNQVSLWCLVKGFYPSDIAVEWESNGQ |
| | | PENNYKTTPPVLDSDGSFFLYSKLTVDKSRWQQGNVFSCSV |
| | | MHEALHNHYTQKSLSLSPGK |
| 7 | p35 (I41V)-15GS- | TPDPGMFPCLHHSQNLLRAVSNMLQKARQTLEFYPCTSEEV |
| | Fc (LALADS-KIH | DHEDITKDKTSTVEACLPLELTKNESCLNSRETSFITNGSCLA |
| | Knob: S354C- | SRKTSFMMALCLSSIYEDLKMYQVEFKTMNAKLLMDPKRQI |
| | T366W) | FLDQNMLAVIDELMQALNFNSETVPQKSSLEEPDFYKTKIKL |
| | | CILLHAFRIRAVTIDRVMSYLNASGGGGSGGGGSGGGGSD |
| | | KTHTCPPCPAPEAAGGPSVFLFPPKPKDTLMISRTPEVTCVVV |
| | | SVSHEDPEVKFNWYVDGVEVHNAKTKPREEQYNSTYRVVS |
| | | VLTVLHQDWLNGKEYKCKVSNKALPAPIEKTISKAKGQPRE |
| | | PQVYTLPPCRDELTKNQVSLWCLVKGFYPSDIAVEWESNGQ |
| | | PENNYKTTPPVLDSDGSFFLYSKLTVDKSRWQQGNVFSCSV |
| | | MHEALHNHYTQKSLSLSPGK |
| 8 | p35 (I41S)-15GS- | TPDPGMFPCLHHSQNLLRAVSNMLQKARQTLEFYPCTSEESD |
| | Fc (LALADS-KIH | HEDITKDKTSTVEACLPLELTKNESCLNSRETSFITNGSCLAS |
| | Knob: S354C- | RKTSFMMALCLSSIYEDLKMYQVEFKTMNAKLLMDPKRQIF |
| | T366W) | LDQNMLAVIDELMQALNFNSETVPQKSSLEEPDFYKTKIKLC |
| | | ILLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 9 | p35 (I41T)-15GS- | TPDPGMFPCLHHSQNLLRAVSNMLQKARQTLEFYPCTSEET |
| | Fc (LALADS-KIH | DHEDITKDKTSTVEACLPLELTKNESCLNSRETSFITNGSCLA |
| | Knob: S354C- | SRKTSFMMALCLSSIYEDLKMYQVEFKTMNAKLLMDPKRQI |
| | T366W) | FLDQNMLAVIDELMQALNFNSETVPQKSSLEEPDFYKTKIKL |
| | | CILLHAFRIRAVTIDRVMSYLNASGGGGSGGGGSGGGGSD |
| | | KTHTCPPCPAPEAAGGPSVFLFPPKPKDTLMISRTPEVTCVVV |
| | | SVSHEDPEVKFNWYVDGVEVHNAKTKPREEQYNSTYRVVS |
| | | VLTVLHQDWLNGKEYKCKVSNKALPAPIEKTISKAKGQPRE |
| | | PQVYTLPPCRDELTKNQVSLWCLVKGFYPSDIAVEWESNGQ |
| | | PENNYKTTPPVLDSDGSFFLYSKLTVDKSRWQQGNVFSCSV |
| | | MHEALHNHYTQKSLSLSPGK |
| 10 | p35 (D42A)- | TPDPGMFPCLHHSQNLLRAVSNMLQKARQTLEFYPCTSEEIA |
| | 15GS-Fc | HEDITKDKTSTVEACLPLELTKNESCLNSRETSFITNGSCLAS |
| | (LALADS-KIH | RKTSFMMALCLSSIYEDLKMYQVEFKTMNAKLLMDPKRQIF |
| | Knob: S354C- | LDQNMLAVIDELMQALNFNSETVPQKSSLEEPDFYKTKIKLC |
| | T366W) | ILLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 11 | p35 (D42L)-15GS- | TPDPGMFPCLHHSQNLLRAVSNMLQKARQTLEFYPCTSEEIL |
| | Fc (LALADS-KIH | HEDITKDKTSTVEACLPLELTKNESCLNSRETSFITNGSCLAS |
| | Knob: S354C- | RKTSFMMALCLSSIYEDLKMYQVEFKTMNAKLLMDPKRQIF |
| | T366W) | LDQNMLAVIDELMQALNFNSETVPQKSSLEEPDFYKTKIKLC |
| | | ILLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 12 | p35 (D42W)- | TPDPGMFPCLHHSQNLLRAVSNMLQKARQTLEFYPCTSEEIW |
| | 15GS-Fc | HEDITKDKTSTVEACLPLELTKNESCLNSRETSFITNGSCLAS |
| | (LALADS-KIH | RKTSFMMALCLSSIYEDLKMYQVEFKTMNAKLLMDPKRQIF |
| | Knob: S354C- | LDQNMLAVIDELMQALNFNSETVPQKSSLEEPDFYKTKIKLC |
| | T366W) | ILLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 13 | p35 (E44A)-15GS- | TPDPGMFPCLHHSQNLLRAVSNMLQKARQTLEFYPCTSEEID |
| | Fc (LALADS-KIH | HADITKDKTSTVEACLPLELTKNESCLNSRETSFITNGSCLAS |
| | Knob: S354C- | RKTSFMMALCLSSIYEDLKMYQVEFKTMNAKLLMDPKRQIF |
| | T366W) | LDQNMLAVIDELMQALNFNSETVPQKSSLEEPDFYKTKIKLC |
| | | ILLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 14 | p35 (E44K)-15GS- | TPDPGMFPCLHHSQNLLRAVSNMLQKARQTLEFYPCTSEEID |
| | Fc (LALADS-KIH | HKDITKDKTSTVEACLPLELTKNESCLNSRETSFITNGSCLAS |
| | Knob: S354C- | RKTSFMMALCLSSIYEDLKMYQVEFKTMNAKLLMDPKRQIF |
| | T366W) | LDQNMLAVIDELMQALNFNSETVPQKSSLEEPDFYKTKIKLC |
| | | ILLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 15 | p35 (E44R)-15GS- | TPDPGMFPCLHHSQNLLRAVSNMLQKARQTLEFYPCTSEEID |
| | Fc (LALADS-KIH | HRDITKDKTSTVEACLPLELTKNESCLNSRETSFITNGSCLAS |
| | Knob: S354C- | RKTSFMMALCLSSIYEDLKMYQVEFKTMNAKLLMDPKRQIF |
| | T366W) | LDQNMLAVIDELMQALNFNSETVPQKSSLEEPDFYKTKIKLC |
| | | ILLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 16 | p35 (D159K)- | TPDPGMFPCLHHSQNLLRAVSNMLQKARQTLEFYPCTSEEID |
| | 15GS-Fc | HEDITKDKTSTVEACLPLELTKNESCLNSRETSFITNGSCLAS |
| | (LALADS-KIH | RKTSFMMALCLSSIYEDLKMYQVEFKTMNAKLLMDPKRQIF |
| | Knob: S354C- | LDQNMLAVIDELMQALNFNSETVPQKSSLEEPKFYKTKIKLC |
| | T366W) | ILLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 17 | p35 (D159R)- | TPDPGMFPCLHHSQNLLRAVSNMLQKARQTLEFYPCTSEEID |
| | 15GS-Fc | HEDITKDKTSTVEACLPLELTKNESCLNSRETSFITNGSCLAS |
| | (LALADS-KIH | RKTSFMMALCLSSIYEDLKMYQVEFKTMNAKLLMDPKRQIF |
| | Knob: S354C- | LDQNMLAVIDELMQALNFNSETVPQKSSLEEPRFYKTKIKLC |
| | T366W) | ILLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 18 | p35 (D159Y)- | TPDPGMFPCLHHSQNLLRAVSNMLQKARQTLEFYPCTSEEID |
| | 15GS-Fc | HEDITKDKTSTVEACLPLELTKNESCLNSRETSFITNGSCLAS |
| | (LALADS-KIH | RKTSFMMALCLSSIYEDLKMYQVEFKTMNAKLLMDPKRQIF |
| | Knob: S354C- | LDQNMLAVIDELMQALNFNSETVPQKSSLEEPYFYKTKIKLC |
| | T366W) | ILLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 19 | p35 (D159F)- | TPDPGMFPCLHHSQNLLRAVSNMLQKARQTLEFYPCTSEEID |
| | 15GS-Fc | HEDITKDKTSTVEACLPLELTKNESCLNSRETSFITNGSCLAS |
| | (LALADS-KIH | RKTSFMMALCLSSIYEDLKMYQVEFKTMNAKLLMDPKRQIF |
| | Knob: S354C- | LDQNMLAVIDELMQALNFNSETVPQKSSLEEPFFYKTKIKLCI |
| | T366W) | LLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 20 | p35 (Y161S)- | TPDPGMFPCLHHSQNLLRAVSNMLQKARQTLEFYPCTSEEID |
| | 15GS-Fc | HEDITKDKTSTVEACLPLELTKNESCLNSRETSFITNGSCLAS |
| | (LALADS)(KIH | RKTSFMMALCLSSIYEDLKMYQVEFKTMNAKLLMDPKRQIF |
| | Knob: S354C- | LDQNMLAVIDELMQALNFNSETVPQKSSLEEPDFSKTKIKLCI |
| | T366W) | LLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 21 | p35 (Y161T)- | TPDPGMFPCLHHSQNLLRAVSNMLQKARQTLEFYPCTSEEID |
| | 15GS-Fc | HEDITKDKTSTVEACLPLELTKNESCLNSRETSFITNGSCLAS |
| | (LALADS-KIH | RKTSFMMALCLSSIYEDLKMYQVEFKTMNAKLLMDPKRQIF |
| | Knob: S354C- | LDQNMLAVIDELMQALNFNSETVPQKSSLEEPDFTKTKIKLCI |
| | T366W) | LLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 22 | p35 (Y161A)- | TPDPGMFPCLHHSQNLLRAVSNMLQKARQTLEFYPCTSEEID |
| | 15GS-Fc | HEDITKDKTSTVEACLPLELTKNESCLNSRETSFITNGSCLAS |
| | (LALADS-KIH | RKTSFMMALCLSSIYEDLKMYQVEFKTMNAKLLMDPKRQIF |
| | Knob: S354C- | LDQNMLAVIDELMQALNFNSETVPQKSSLEEPDFAKTKIKLC |
| | T366W) | ILLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 23 | p35 (Y161W)- | TPDPGMFPCLHHSQNLLRAVSNMLQKARQTLEFYPCTSEEID |
| | 15GS-Fc | HEDITKDKTSTVEACLPLELTKNESCLNSRETSFITNGSCLAS |
| | (LALADS-KIH | RKTSFMMALCLSSIYEDLKMYQVEFKTMNAKLLMDPKRQIF |
| | Knob: S354C- | LDQNMLAVIDELMQALNFNSETVPQKSSLEEPDFWKTKIKLC |
| | T366W) | ILLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 24 | p35 (K162E)- | TPDPGMFPCLHHSQNLLRAVSNMLQKARQTLEFYPCTSEEID |
| | 15GS-Fc | HEDITKDKTSTVEACLPLELTKNESCLNSRETSFITNGSCLAS |
| | (LALADS-KIH | RKTSFMMALCLSSIYEDLKMYQVEFKTMNAKLLMDPKRQIF |
| | Knob: S354C- | LDQNMLAVIDELMQALNFNSETVPQKSSLEEPDFYETKIKLCI |
| | T366W) | LLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 25 | p35 (K162D)- | TPDPGMFPCLHHSQNLLRAVSNMLQKARQTLEFYPCTSEEID |
| | 15GS-Fc | HEDITKDKTSTVEACLPLELTKNESCLNSRETSFITNGSCLAS |
| | (LALADS-KIH | RKTSFMMALCLSSIYEDLKMYQVEFKTMNAKLLMDPKRQIF |
| | Knob: S354C- | LDQNMLAVIDELMQALNFNSETVPQKSSLEEPDFYDTKIKLC |
| | T366W) | ILLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 26 | p40 (K85E)-4G-Fc | IWELKKDVYVVELDWYPDAPGEMVVLTCDTPEEDGITWTL |
| | (LALADS-KIH | DQSSEVLGSGKTLTIQVKEFGDAGQYTCHKGGEVLSHSLLLL |
| | Hole: Y349C- | HKEEDGIWSTDILKDQKEPKNKTFLRCEAKNYSGRFTCWWL |
| | T366S-L368A- | TTISTDLTFSVKSSRGSSDPQGVTCGAATLSAERVRGDNKEY |
| | Y407V) | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLMISRTPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 27 | p40 (K85Y)-4G- | IWELKKDVYVVELDWYPDAPGEMVVLTCDTPEEDGITWTL |
| | Fc (LALADS-KIH | DQSSEVLGSGKTLTIQVKEFGDAGQYTCHKGGEVLSHSLLLL |
| | Hole: Y349C- | HKYEDGIWSTDILKDQKEPKNKTFLRCEAKNYSGRFTCWWL |
| | T366S-L368A- | TTISTDLTFSVKSSRGSSDPQGVTCGAATLSAERVRGDNKEY |
| | Y407V) | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLMISRTPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 28 | p40 (D87A)-4G- | IWELKKDVYVVELDWYPDAPGEMVVLTCDTPEEDGITWTL |
| | Fc (LALADS-KIH | DQSSEVLGSGKTLTIQVKEFGDAGQYTCHKGGEVLSHSLLLL |
| | Hole: Y349C- | HKKEAGIWSTDILKDQKEPKNKTFLRCEAKNYSGRFTCWWL |
| | T366S-L368A- | TTISTDLTFSVKSSRGSSDPQGVTCGAATLSAERVRGDNKEY |
| | Y407V) | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLMISRTPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 29 | p40 (D87L)-4G-Fc | IWELKKDVYVVELDWYPDAPGEMVVLTCDTPEEDGITWTL |
| | (LALADS-KIH | DQSSEVLGSGKTLTIQVKEFGDAGQYTCHKGGEVLSHSLLLL |
| | Hole: Y349C- | HKKELGIWSTDILKDQKEPKNKTFLRCEAKNYSGRFTCWWL |
| | T366S-L368A- | TTISTDLTFSVKSSRGSSDPQGVTCGAATLSAERVRGDNKEY |
| | Y407V) | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLMISRTPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 30 | p40 (I89A)-4G-Fc | IWELKKDVYVVELDWYPDAPGEMVVLTCDTPEEDGITWTL |
| | (LALADS-KIH | DQSSEVLGSGKTLTIQVKEFGDAGQYTCHKGGEVLSHSLLLL |
| | Hole: Y349C- | HKKEDGAWSTDILKDQKEPKNKTFLRCEAKNYSGRFTCWW |
| | T366S-L368A- | LTTISTDLTFSVKSSRGSSDPQGVTCGAATLSAERVRGDNKE |
| | Y407V) | YEYSVECQEDSACPAAEESLPIEVMVDAVHKLKYENYTSSFF |
| | | IRDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLMISRTPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 31 | p40 (I89S)-4G-Fc | IWELKKDVYVVELDWYPDAPGEMVVLTCDTPEEDGITWTL |
| | (LALADS-KIH | DQSSEVLGSGKTLTIQVKEFGDAGQYTCHKGGEVLSHSLLLL |
| | Hole: Y349C- | HKKEDGSWSTDILKDQKEPKNKTFLRCEAKNYSGRFTCWW |
| | T366S-L368A- | LTTISTDLTFSVKSSRGSSDPQGVTCGAATLSAERVRGDNKE |
| | Y407V) | YEYSVECQEDSACPAAEESLPIEVMVDAVHKLKYENYTSSFF |
| | | IRDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLMISRTPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 32 | p40 (I89D)-4G-Fc | IWELKKDVYVVELDWYPDAPGEMVVLTCDTPEEDGITWTL |
| | (LALADS-KIH | DQSSEVLGSGKTLTIQVKEFGDAGQYTCHKGGEVLSHSLLLL |
| | Hole: Y349C- | HKKEDGDWSTDILKDQKEPKNKTFLRCEAKNYSGRFTCWW |
| | T366S-L368A- | LTTISTDLTFSVKSSRGSSDPQGVTCGAATLSAERVRGDNKE |
| | Y407V) | YEYSVECQEDSACPAAEESLPIEVMVDAVHKLKYENYTSSFF |
| | | IRDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLMISRTPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 33 | p40 (I89P)-4G-Fc | IWELKKDVYVVELDWYPDAPGEMVVLTCDTPEEDGITWTL |
| | (LALADS-KIH | DQSSEVLGSGKTLTIQVKEFGDAGQYTCHKGGEVLSHSLLLL |
| | Hole: Y349C- | HKKEDGPWSTDILKDQKEPKNKTFLRCEAKNYSGRFTCWW |
| | T366S-L368A- | LTTISTDLTFSVKSSRGSSDPQGVTCGAATLSAERVRGDNKE |
| | Y407V) | YEYSVECQEDSACPAAEESLPIEVMVDAVHKLKYENYTSSFF |
| | | IRDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLMISRTPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 34 | p40 (K104E)-4G- | IWELKKDVYVVELDWYPDAPGEMVVLTCDTPEEDGITWTL |
| | Fc (LALADS-KIH | DQSSEVLGSGKTLTIQVKEFGDAGQYTCHKGGEVLSHSLLLL |
| | Hole: Y349C- | HKKEDGIWSTDILKDQKEPKNETFLRCEAKNYSGRFTCWWL |
| | T366S-L368A- | TTISTDLTFSVKSSRGSSDPQGVTCGAATLSAERVRGDNKEY |
| | Y407V) | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLMISRTPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 35 | p40 (K104D)-4G- | IWELKKDVYVVELDWYPDAPGEMVVLTCDTPEEDGITWTL |
| | Fc (LALADS-KIH | DQSSEVLGSGKTLTIQVKEFGDAGQYTCHKGGEVLSHSLLLL |
| | Hole: Y349C- | HKKEDGIWSTDILKDQKEPKNDTFLRCEAKNYSGRFTCWWL |
| | T366S-L368A- | TTISTDLTFSVKSSRGSSDPQGVTCGAATLSAERVRGDNKEY |
| | Y407V) | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLMISRTPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 36 | p40 (K104W)-4G- | IWELKKDVYVVELDWYPDAPGEMVVLTCDTPEEDGITWTL |
| | Fc (LALADS-KIH | DQSSEVLGSGKTLTIQVKEFGDAGQYTCHKGGEVLSHSLLLL |
| | Hole: Y349C- | HKKEDGIWSTDILKDQKEPKNWTFLRCEAKNYSGRFTCWW |
| | T366S-L368A- | LTTISTDLTFSVKSSRGSSDPQGVTCGAATLSAERVRGDNKE |
| | Y407V) | YEYSVECQEDSACPAAEESLPIEVMVDAVHKLKYENYTSSFF |
| | | IRDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLMISRTPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 37 | p40 (K104S)-4G- | IWELKKDVYVVELDWYPDAPGEMVVLTCDTPEEDGITWTL |
| | Fc (LALADS-KIH | DQSSEVLGSGKTLTIQVKEFGDAGQYTCHKGGEVLSHSLLLL |
| | Hole: Y349C- | HKKEDGIWSTDILKDQKEPKNSTFLRCEAKNYSGRFTCWWL |
| | T366S-L368A- | TTISTDLTFSVKSSRGSSDPQGVTCGAATLSAERVRGDNKEY |
| | Y407V) | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLMISRTPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 38 | p35 (D159K- | TPDPGMFPCLHHSQNLLRAVSNMLQKARQTLEFYPCTSEEID |
| | Y161T)-15GS-Fc | HEDITKDKTSTVEACLPLELTKNESCLNSRETSFITNGSCLAS |
| | (LALADS-KIH | RKTSFMMALCLSSIYEDLKMYQVEFKTMNAKLLMDPKRQIF |
| | Knob: S354C- | LDQNMLAVIDELMQALNFNSETVPQKSSLEEPKFTKTKIKLCI |
| | T366W) | LLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 39 | p35 (F33G- | TPDPGMFPCLHHSQNLLRAVSNMLQKARQTLEGYPCTSEEID |
| | D159K-Y161T)- | HEDITKDKTSTVEACLPLELTKNESCLNSRETSFITNGSCLAS |
| | 15GS-Fc | RKTSFMMALCLSSIYEDLKMYQVEFKTMNAKLLMDPKRQIF |
| | (LALADS-KIH | LDQNMLAVIDELMQALNFNSETVPQKSSLEEPKFTKTKIKLCI |
| | Knob: S354C- | LLHAFRIRAVTIDRVMSYLNASGGGGSGGGGSGGGGSDKT |
| | T366W) | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 40 | p35 (F33K- | TPDPGMFPCLHHSQNLLRAVSNMLQKARQTLEKYPCTSEEID |
| | D159K-Y161T)- | HEDITKDKTSTVEACLPLELTKNESCLNSRETSFITNGSCLAS |
| | 15GS-Fc | RKTSFMMALCLSSIYEDLKMYQVEFKTMNAKLLMDPKRQIF |
| | (LALADS-KIH | LDQNMLAVIDELMQALNFNSETVPQKSSLEEPKFTKTKIKLCI |
| | Knob: S354C- | LLHAFRIRAVTIDRVMSYLNASGGGGSGGGGSGGGGSDKT |
| | T366W) | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 41 | p35 (F33S- | TPDPGMFPCLHHSQNLLRAVSNMLQKARQTLESYPCTSEEID |
| | D159K-Y161T)- | HEDITKDKTSTVEACLPLELTKNESCLNSRETSFITNGSCLAS |
| | 15GS-Fc | RKTSFMMALCLSSIYEDLKMYQVEFKTMNAKLLMDPKRQIF |
| | (LALADS-KIH | LDQNMLAVIDELMQALNFNSETVPQKSSLEEPKFTKTKIKLCI |
| | Knob: S354C- | LLHAFRIRAVTIDRVMSYLNASGGGGSGGGGSGGGGSDKT |
| | T366W) | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 42 | p35 (Y34G- | TPDPGMFPCLHHSQNLLRAVSNMLQKARQTLEFGPCTSEEID |
| | D159K-Y161T)- | HEDITKDKTSTVEACLPLELTKNESCLNSRETSFITNGSCLAS |
| | 15GS-Fc | RKTSFMMALCLSSIYEDLKMYQVEFKTMNAKLLMDPKRQIF |
| | (LALADS-KIH | LDQNMLAVIDELMQALNFNSETVPQKSSLEEPKFTKTKIKLCI |
| | Knob: S354C- | LLHAFRIRAVTIDRVMSYLNASGGGGSGGGGSGGGGSDKT |
| | T366W) | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 43 | p35 (Y34S- | TPDPGMFPCLHHSQNLLRAVSNMLQKARQTLEFSPCTSEEID |
| | D159K-Y161T)- | HEDITKDKTSTVEACLPLELTKNESCLNSRETSFITNGSCLAS |
| | 15GS-Fc | RKTSFMMALCLSSIYEDLKMYQVEFKTMNAKLLMDPKRQIF |
| | (LALADS-KIH | LDQNMLAVIDELMQALNFNSETVPQKSSLEEPKFTKTKIKLCI |
| | Knob: S354C- | LLHAFRIRAVTIDRVMSYLNASGGGGSGGGGSGGGGSDKT |
| | T366W) | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 44 | p35 (Y34T- | TPDPGMFPCLHHSQNLLRAVSNMLQKARQTLEFTPCTSEEID |
| | D159K-Y161T)- | HEDITKDKTSTVEACLPLELTKNESCLNSRETSFITNGSCLAS |
| | 15GS-Fc | RKTSFMMALCLSSIYEDLKMYQVEFKTMNAKLLMDPKRQIF |
| | (LALADS-KIH | LDQNMLAVIDELMQALNFNSETVPQKSSLEEPKFTKTKIKLCI |
| | Knob: S354C- | LLHAFRIRAVTIDRVMSYLNASGGGGSGGGGSGGGGSDKT |
| | T366W) | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 45 | p35 (P35Q- | TPDPGMFPCLHHSQNLLRAVSNMLQKARQTLEFYQCTSEEID |
| | D159K-Y161T)- | HEDITKDKTSTVEACLPLELTKNESCLNSRETSFITNGSCLAS |
| | 15GS-Fc | RKTSFMMALCLSSIYEDLKMYQVEFKTMNAKLLMDPKRQIF |
| | (LALADS-KIH | LDQNMLAVIDELMQALNFNSETVPQKSSLEEPKFTKTKIKLCI |
| | Knob: S354C- | LLHAFRIRAVTIDRVMSYLNASGGGGSGGGGSGGGGSDKT |
| | T366W) | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 46 | p35 (P35E- | TPDPGMFPCLHHSQNLLRAVSNMLQKARQTLEFYECTSEEID |
| | D159K-Y161T)- | HEDITKDKTSTVEACLPLELTKNESCLNSRETSFITNGSCLAS |
| | 15GS-Fc | RKTSFMMALCLSSIYEDLKMYQVEFKTMNAKLLMDPKRQIF |
| | (LALADS-KIH | LDQNMLAVIDELMQALNFNSETVPQKSSLEEPKFTKTKIKLCI |
| | Knob: S354C- | LLHAFRIRAVTIDRVMSYLNASGGGGSGGGGSGGGGSDKT |
| | T366W) | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 47 | p35 (F33S-Y34T | TPDPGMFPCLHHSQNLLRAVSNMLQKARQTLESTPCTSEEID |
| | D159K-Y161T)- | HEDITKDKTSTVEACLPLELTKNESCLNSRETSFITNGSCLAS |
| | 15GS-Fc | RKTSFMMALCLSSIYEDLKMYQVEFKTMNAKLLMDPKRQIF |
| | (LALADS-KIH | LDQNMLAVIDELMQALNFNSETVPQKSSLEEPKFTKTKIKLCI |
| | Knob: S354C- | LLHAFRIRAVTIDRVMSYLNASGGGGSGGGGSGGGGSDKT |
| | T366W) | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 48 | p35 (F33S-Y34S | TPDPGMFPCLHHSQNLLRAVSNMLQKARQTLESSPCTSEEID |
| | D159K-Y161T)- | HEDITKDKTSTVEACLPLELTKNESCLNSRETSFITNGSCLAS |
| | 15GS-Fc | RKTSFMMALCLSSIYEDLKMYQVEFKTMNAKLLMDPKRQIF |
| | (LALADS-KIH | LDQNMLAVIDELMQALNFNSETVPQKSSLEEPKFTKTKIKLCI |
| | Knob: S354C- | LLHAFRIRAVTIDRVMSYLNASGGGGSGGGGSGGGGSDKT |
| | T366W) | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 49 | p35 (F33G- | TPDPGMFPCLHHSQNLLRAVSNMLQKARQTLEGYPCTSEEID |
| | Y161T)-15GS-Fc | HEDITKDKTSTVEACLPLELTKNESCLNSRETSFITNGSCLAS |
| | (LALADS-KIH | RKTSFMMALCLSSIYEDLKMYQVEFKTMNAKLLMDPKRQIF |
| | Knob: S354C- | LDQNMLAVIDELMQALNFNSETVPQKSSLEEPDFTKTKIKLCI |
| | T366W) | LLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 50 | p35 (F33K- | TPDPGMFPCLHHSQNLLRAVSNMLQKARQTLEKYPCTSEEID |
| | Y161T)-15GS-Fc | HEDITKDKTSTVEACLPLELTKNESCLNSRETSFITNGSCLAS |
| | (LALADS-KIH | RKTSFMMALCLSSIYEDLKMYQVEFKTMNAKLLMDPKRQIF |
| | Knob: S354C- | LDQNMLAVIDELMQALNFNSETVPQKSSLEEPDFTKTKIKLCI |
| | T366W) | LLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 51 | p35 (F33S-Y161T)- | TPDPGMFPCLHHSQNLLRAVSNMLQKARQTLESYPCTSEEID |
| | 15GS-Fc | HEDITKDKTSTVEACLPLELTKNESCLNSRETSFITNGSCLAS |
| | (LALADS-KIH | RKTSFMMALCLSSIYEDLKMYQVEFKTMNAKLLMDPKRQIF |
| | Knob: S354C- | LDQNMLAVIDELMQALNFNSETVPQKSSLEEPDFTKTKIKLCI |
| | T366W) | LLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 52 | p35 (Y34G- | TPDPGMFPCLHHSQNLLRAVSNMLQKARQTLEFGPCTSEEID |
| | Y161T)-15GS-Fc | HEDITKDKTSTVEACLPLELTKNESCLNSRETSFITNGSCLAS |
| | (LALADS-KIH | RKTSFMMALCLSSIYEDLKMYQVEFKTMNAKLLMDPKRQIF |
| | Knob: S354C- | LDQNMLAVIDELMQALNFNSETVPQKSSLEEPDFTKTKIKLCI |
| | T366W) | LLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 53 | p35 (Y34S- | TPDPGMFPCLHHSQNLLRAVSNMLQKARQTLEFSPCTSEEID |
| | Y161T)-15GS-Fc | HEDITKDKTSTVEACLPLELTKNESCLNSRETSFITNGSCLAS |
| | (LALADS-KIH | RKTSFMMALCLSSIYEDLKMYQVEFKTMNAKLLMDPKRQIF |
| | Knob: S354C- | LDQNMLAVIDELMQALNFNSETVPQKSSLEEPDFTKTKIKLCI |
| | T366W) | LLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 54 | p35 (Y34T- | TPDPGMFPCLHHSQNLLRAVSNMLQKARQTLEFTPCTSEEID |
| | Y161T)-15GS-Fc | HEDITKDKTSTVEACLPLELTKNESCLNSRETSFITNGSCLAS |
| | (LALADS-KIH | RKTSFMMALCLSSIYEDLKMYQVEFKTMNAKLLMDPKRQIF |
| | Knob: S354C- | LDQNMLAVIDELMQALNFNSETVPQKSSLEEPDFTKTKIKLCI |
| | T366W) | LLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 55 | p35 (F33S-Y34T- | TPDPGMFPCLHHSQNLLRAVSNMLQKARQTLESTPCTSEEID |
| | Y161T)-15GS-Fc | HEDITKDKTSTVEACLPLELTKNESCLNSRETSFITNGSCLAS |
| | (LALADS-KIH | RKTSFMMALCLSSIYEDLKMYQVEFKTMNAKLLMDPKRQIF |
| | Knob: S354C- | LDQNMLAVIDELMQALNFNSETVPQKSSLEEPDFTKTKIKLCI |
| | T366W) | LLHAFRIRAVTIDRVMSYLNASGGGGGSGGGGSGGGGSDK |
| | | THTCPPCPAPEAAGGPSVFLFPPKPKDTLMISRTPEVTCVVVS |
| | | VSHEDPEVKFNWYVDGVEVHNAKTKPREEQYNSTYRVVSV |
| | | LTVLHQDWLNGKEYKCKVSNKALPAPIEKTISKAKGQPREP |
| | | QVYTLPPCRDELTKNQVSLWCLVKGFYPSDIAVEWESNGQP |
| | | ENNYKTTPPVLDSDGSFFLYSKLTVDKSRWQQGNVFSCSVM |
| | | HEALHNHYTQKSLSLSPGK |
| 56 | p35 (F33S-Y34S- | TPDPGMFPCLHHSQNLLRAVSNMLQKARQTLESSPCTSEEID |
| | Y161T)-15GS-Fc | HEDITKDKTSTVEACLPLELTKNESCLNSRETSFITNGSCLAS |
| | (LALADS-KIH | RKTSFMMALCLSSIYEDLKMYQVEFKTMNAKLLMDPKRQIF |
| | Knob: S354C- | LDQNMLAVIDELMQALNFNSETVPQKSSLEEPDFTKTKIKLCI |
| | T366W) | LLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 57 | p35 (F33S-Y34G- | TPDPGMFPCLHHSQNLLRAVSNMLQKARQTLESGPCTSEEID |
| | Y161T)-15GS-Fc | HEDITKDKTSTVEACLPLELTKNESCLNSRETSFITNGSCLAS |
| | (LALADS-KIH | RKTSFMMALCLSSIYEDLKMYQVEFKTMNAKLLMDPKRQIF |
| | Knob: S354C- | LDQNMLAVIDELMQALNFNSETVPQKSSLEEPDFTKTKIKLCI |
| | T366W) | LLHAFRIRAVTIDRVMSYLNASGGGGSGGGGSGGGGSDKT |
| | | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 58 | p40 (D87L- | IWELKKDVYVVELDWYPDAPGEMVVLTCDTPEEDGITWTL |
| | K104E)-4G-Fc | DQSSEVLGSGKTLTIQVKEFGDAGQYTCHKGGEVLSHSLLLL |
| | (LALADS-KIH | HKKELGIWSTDILKDQKEPKNETFLRCEAKNYSGRFTCWWL |
| | Hole: Y349C- | TTISTDLTFSVKSSRGSSDPQGVTCGAATLSAERVRGDNKEY |
| | T366S-L368A- | EYSVECQEDSACPAAEESLPIEVMVDAVHKLKYENYTSSFFI |
| | Y407V) | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLMISRTPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 59 | p40 (D87L- | IWELKKDVYVVELDWYPDAPGEMVVLTCDTPEEDGITWTL |
| | K104D)-4G-Fc | DQSSEVLGSGKTLTIQVKEFGDAGQYTCHKGGEVLSHSLLLL |
| | (LALADS-KIH | HKKELGIWSTDILKDQKEPKNDTFLRCEAKNYSGRFTCWWL |
| | Hole: Y349C- | TTISTDLTFSVKSSRGSSDPQGVTCGAATLSAERVRGDNKEY |
| | T366S-L368A- | EYSVECQEDSACPAAEESLPIEVMVDAVHKLKYENYTSSFFI |
| | Y407V) | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLMISRTPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 60 | p40 (P143A- | IWELKKDVYVVELDWYPDAPGEMVVLTCDTPEEDGITWTL |
| | P215A)-4G-Fc | DQSSEVLGSGKTLTIQVKEFGDAGQYTCHKGGEVLSHSLLLL |
| | (LALADS-KIH | HKKEDGIWSTDILKDQKEPKNKTFLRCEAKNYSGRFTCWWL |
| | Hole: Y349C- | TTISTDLTFSVKSSRGSSDAQGVTCGAATLSAERVRGDNKEY |
| | T366S-L368A- | EYSVECQEDSACPAAEESLPIEVMVDAVHKLKYENYTSSFFI |
| | Y407V) | RDIIKPDAPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLMISRTPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 61 | p40 (P143A- | IWELKKDVYVVELDWYPDAPGEMVVLTCDTPEEDGITWTL |
| | P215A-K260A- | DQSSEVLGSGKTLTIQVKEFGDAGQYTCHKGGEVLSHSLLLL |
| | R261S-V303I)- | HKKEDGIWSTDILKDQKEPKNKTFLRCEAKNYSGRFTCWWL |
| | 4G-Fc (LALADS- | TTISTDLTFSVKSSRGSSDAQGVTCGAATLSAERVRGDNKEY |
| | KIH Hole: Y349C- | EYSVECQEDSACPAAEESLPIEVMVDAVHKLKYENYTSSFFI |
| | T366S-L368A- | RDIIKPDAPKNLQLKPLKNSRQVEVSWEYPDTWSTPHSYFSL |
| | Y407V) | TFCVQVQGKSASEKKDRVFTDKTSATVICRKNASISVRAQDR |
| | | YYSSSWSEWASIPCSGGGGDKTHTCPPCPAPEAAGGPSVFLF |
| | | PPKPKDTLMISRTPEVTCVVVSVSHEDPEVKFNWYVDGVEV |
| | | HNAKTKPREEQYNSTYRVVSVLTVLHQDWLNGKEYKCKVS |
| | | NKALPAPIEKTISKAKGQPREPQVCTLPPSRDELTKNQVSLSC |
| | | AVKGFYPSDIAVEWESNGQPENNYKTTPPVLDSDGSFFLVSK |
| | | LTVDKSRWQQGNVFSCSVMHEALHNHYTQKSLSLSPGK |
| 62 | p40 (P143A- | IWELKKDVYVVELDWYPDAPGEMVVLTCDTPEEDGITWTL |
| | P215A-K260S- | DQSSEVLGSGKTLTIQVKEFGDAGQYTCHKGGEVLSHSLLLL |
| | R261S-V303I)- | HKKEDGIWSTDILKDQKEPKNKTFLRCEAKNYSGRFTCWWL |
| | 4G-Fc (LALADS- | TTISTDLTFSVKSSRGSSDAQGVTCGAATLSAERVRGDNKEY |
| | KIH Hole: Y349C- | EYSVECQEDSACPAAEESLPIEVMVDAVHKLKYENYTSSFFI |
| | T366S-L368A- | RDIIKPDAPKNLQLKPLKNSRQVEVSWEYPDTWSTPHSYFSL |
| | Y407V) | TFCVQVQGKSSSEKKDRVFTDKTSATVICRKNASISVRAQDR |
| | | YYSSSWSEWASIPCSGGGGDKTHTCPPCPAPEAAGGPSVFLF |
| | | PPKPKDTLMISRTPEVTCVVVSVSHEDPEVKFNWYVDGVEV |
| | | HNAKTKPREEQYNSTYRVVSVLTVLHQDWLNGKEYKCKVS |
| | | NKALPAPIEKTISKAKGQPREPQVCTLPPSRDELTKNQVSLSC |
| | | AVKGFYPSDIAVEWESNGQPENNYKTTPPVLDSDGSFFLVSK |
| | | LTVDKSRWQQGNVFSCSVMHEALHNHYTQKSLSLSPGK |
| 63 | p40 (K260A- | IWELKKDVYVVELDWYPDAPGEMVVLTCDTPEEDGITWTL |
| | R261S-V303I)- | DQSSEVLGSGKTLTIQVKEFGDAGQYTCHKGGEVLSHSLLLL |
| | 4G-Fc (LALADS- | HKKEDGIWSTDILKDQKEPKNKTFLRCEAKNYSGRFTCWWL |
| | KIH Hole: Y349C- | TTISTDLTFSVKSSRGSSDPQGVTCGAATLSAERVRGDNKEY |
| | T366S-L368A- | EYSVECQEDSACPAAEESLPIEVMVDAVHKLKYENYTSSFFI |
| | Y407V) | RDIIKPDPPKNLQLKPLKNSRQVEVSWEYPDTWSTPHSYFSL |
| | | TFCVQVQGKSASEKKDRVFTDKTSATVICRKNASISVRAQDR |
| | | YYSSSWSEWASIPCSGGGGDKTHTCPPCPAPEAAGGPSVFLF |
| | | PPKPKDTLMISRTPEVTCVVVSVSHEDPEVKFNWYVDGVEV |
| | | HNAKTKPREEQYNSTYRVVSVLTVLHQDWLNGKEYKCKVS |
| | | NKALPAPIEKTISKAKGQPREPQVCTLPPSRDELTKNQVSLSC |
| | | AVKGFYPSDIAVEWESNGQPENNYKTTPPVLDSDGSFFLVSK |
| | | LTVDKSRWQQGNVFSCSVMHEALHNHYTQKSLSLSPGK |
| 64 | p40 (K260S- | IWELKKDVYVVELDWYPDAPGEMVVLTCDTPEEDGITWTL |
| | R261S-V303I)- | DQSSEVLGSGKTLTIQVKEFGDAGQYTCHKGGEVLSHSLLLL |
| | 4G-Fc (LALADS- | HKKEDGIWSTDILKDQKEPKNKTFLRCEAKNYSGRFTCWWL |
| | KIH Hole: Y349C- | TTISTDLTFSVKSSRGSSDPQGVTCGAATLSAERVRGDNKEY |
| | T366S-L368A- | EYSVECQEDSACPAAEESLPIEVMVDAVHKLKYENYTSSFFI |
| | Y407V) | RDIIKPDPPKNLQLKPLKNSRQVEVSWEYPDTWSTPHSYFSL |
| | | TFCVQVQGKSSSEKKDRVFTDKTSATVICRKNASISVRAQDR |
| | | YYSSSWSEWASIPCSGGGGDKTHTCPPCPAPEAAGGPSVFLF |
| | | PPKPKDTLMISRTPEVTCVVVSVSHEDPEVKFNWYVDGVEV |
| | | HNAKTKPREEQYNSTYRVVSVLTVLHQDWLNGKEYKCKVS |
| | | NKALPAPIEKTISKAKGQPREPQVCTLPPSRDELTKNQVSLSC |
| | | AVKGFYPSDIAVEWESNGQPENNYKTTPPVLDSDGSFFLVSK |
| | | LTVDKSRWQQGNVFSCSVMHEALHNHYTQKSLSLSPGK |
| 65 | p40 (K260A- | IWELKKDVYVVELDWYPDAPGEMVVLTCDTPEEDGITWTL |
| | R261S)-4G-Fc | DQSSEVLGSGKTLTIQVKEFGDAGQYTCHKGGEVLSHSLLLL |
| | (LALADS-KIH | HKKEDGIWSTDILKDQKEPKNKTFLRCEAKNYSGRFTCWWL |
| | Hole: Y349C- | TTISTDLTFSVKSSRGSSDPQGVTCGAATLSAERVRGDNKEY |
| | T366S-L368A- | EYSVECQEDSACPAAEESLPIEVMVDAVHKLKYENYTSSFFI |
| | Y407V) | RDIIKPDPPKNLQLKPLKNSRQVEVSWEYPDTWSTPHSYFSL |
| | | TFCVQVQGKSASEKKDRVFTDKTSATVICRKNASISVRAQDR |
| | | YYSSSWSEWASVPCSGGGGDKTHTCPPCPAPEAAGGPSVFL |
| | | FPPKPKDTLMISRTPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 66 | p40 (K260S- | IWELKKDVYVVELDWYPDAPGEMVVLTCDTPEEDGITWTL |
| | R261S)-4G-Fc | DQSSEVLGSGKTLTIQVKEFGDAGQYTCHKGGEVLSHSLLLL |
| | (LALADS-KIH | HKKEDGIWSTDILKDQKEPKNKTFLRCEAKNYSGRFTCWWL |
| | Hole: Y349C- | TTISTDLTFSVKSSRGSSDPQGVTCGAATLSAERVRGDNKEY |
| | T366S-L368A- | EYSVECQEDSACPAAEESLPIEVMVDAVHKLKYENYTSSFFI |
| | Y407V) | RDIIKPDPPKNLQLKPLKNSRQVEVSWEYPDTWSTPHSYFSL |
| | | TFCVQVQGKSSSEKKDRVFTDKTSATVICRKNASISVRAQDR |
| | | YYSSSWSEWASVPCSGGGGDKTHTCPPCPAPEAAGGPSVFL |
| | | FPPKPKDTLMISRTPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 67 | P35-15GS-Fc | TPDPGMFPCLHHSQNLLRAVSNMLQKARQTLEFYPCTSEEID |
| | (LALADS-YTE- | HEDITKDKTSTVEACLPLELTKNESCLNSRETSFITNGSCLAS |
| | KIH Knob: | RKTSFMMALCLSSIYEDLKMYQVEFKTMNAKLLMDPKRQIF |
| | S354C-T366W) | LDQNMLAVIDELMQALNFNSETVPQKSSLEEPDFYKTKIKLC |
| | | ILLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 68 | P40-4G-Fc | IWELKKDVYVVELDWYPDAPGEMVVLTCDTPEEDGITWTL |
| | (LALADS-YTE- | DQSSEVLGSGKTLTIQVKEFGDAGQYTCHKGGEVLSHSLLLL |
| | KIH Hole: Y349C- | HKKEDGIWSTDILKDQKEPKNKTFLRCEAKNYSGRFTCWWL |
| | T366S-L368A- | TTISTDLTFSVKSSRGSSDPQGVTCGAATLSAERVRGDNKEY |
| | Y407V) | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLYITREPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 69 | p35 (E40K)-15GS- | TPDPGMFPCLHHSQNLLRAVSNMLQKARQTLEFYPCTSEKID |
| | Fc (LALADS- | HEDITKDKTSTVEACLPLELTKNESCLNSRETSFITNGSCLAS |
| | YTE-KIH Knob: | RKTSFMMALCLSSIYEDLKMYQVEFKTMNAKLLMDPKRQIF |
| | S354C-T366W) | LDQNMLAVIDELMQALNFNSETVPQKSSLEEPDFYKTKIKLC |
| | | ILLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 70 | p35 (E40R)-15GS- | TPDPGMFPCLHHSQNLLRAVSNMLQKARQTLEFYPCTSERID |
| | Fc (LALADS- | HEDITKDKTSTVEACLPLELTKNESCLNSRETSFITNGSCLAS |
| | YTE-KIH Knob: | RKTSFMMALCLSSIYEDLKMYQVEFKTMNAKLLMDPKRQIF |
| | S354C-T366W) | LDQNMLAVIDELMQALNFNSETVPQKSSLEEPDFYKTKIKLC |
| | | ILLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 71 | p35 (E40W)- | TPDPGMFPCLHHSQNLLRAVSNMLQKARQTLEFYPCTSEWI |
| | 15GS-Fc | DHEDITKDKTSTVEACLPLELTKNESCLNSRETSFITNGSCLA |
| | (LALADS-YTE- | SRKTSFMMALCLSSIYEDLKMYQVEFKTMNAKLLMDPKRQI |
| | KIH Knob: | FLDQNMLAVIDELMQALNFNSETVPQKSSLEEPDFYKTKIKL |
| | S354C-T366W) | CILLHAFRIRAVTIDRVMSYLNASGGGGSGGGGSGGGGSD |
| | | KTHTCPPCPAPEAAGGPSVFLFPPKPKDTLYITREPEVTCVVV |
| | | SVSHEDPEVKFNWYVDGVEVHNAKTKPREEQYNSTYRVVS |
| | | VLTVLHQDWLNGKEYKCKVSNKALPAPIEKTISKAKGQPRE |
| | | PQVYTLPPCRDELTKNQVSLWCLVKGFYPSDIAVEWESNGQ |
| | | PENNYKTTPPVLDSDGSFFLYSKLTVDKSRWQQGNVFSCSV |
| | | MHEALHNHYTQKSLSLSPGK |
| 72 | p35 (I41A)-15GS- | TPDPGMFPCLHHSQNLLRAVSNMLQKARQTLEFYPCTSEEA |
| | Fc (LALADS- | DHEDITKDKTSTVEACLPLELTKNESCLNSRETSFITNGSCLA |
| | YTE-KIH Knob: | SRKTSFMMALCLSSIYEDLKMYQVEFKTMNAKLLMDPKRQI |
| | S354C-T366W) | FLDQNMLAVIDELMQALNFNSETVPQKSSLEEPDFYKTKIKL |
| | | CILLHAFRIRAVTIDRVMSYLNASGGGGSGGGGSGGGGSD |
| | | KTHTCPPCPAPEAAGGPSVFLFPPKPKDTLYITREPEVTCVVV |
| | | SVSHEDPEVKFNWYVDGVEVHNAKTKPREEQYNSTYRVVS |
| | | VLTVLHQDWLNGKEYKCKVSNKALPAPIEKTISKAKGQPRE |
| | | PQVYTLPPCRDELTKNQVSLWCLVKGFYPSDIAVEWESNGQ |
| | | PENNYKTTPPVLDSDGSFFLYSKLTVDKSRWQQGNVFSCSV |
| | | MHEALHNHYTQKSLSLSPGK |
| 73 | p35 (I41V)-15GS- | TPDPGMFPCLHHSQNLLRAVSNMLQKARQTLEFYPCTSEEV |
| | Fc (LALADS- | DHEDITKDKTSTVEACLPLELTKNESCLNSRETSFITNGSCLA |
| | YTE-KIH Knob: | SRKTSFMMALCLSSIYEDLKMYQVEFKTMNAKLLMDPKRQI |
| | S354C-T366W) | FLDQNMLAVIDELMQALNFNSETVPQKSSLEEPDFYKTKIKL |
| | | CILLHAFRIRAVTIDRVMSYLNASGGGGSGGGGSGGGGSD |
| | | KTHTCPPCPAPEAAGGPSVFLFPPKPKDTLYITREPEVTCVVV |
| | | SVSHEDPEVKFNWYVDGVEVHNAKTKPREEQYNSTYRVVS |
| | | VLTVLHQDWLNGKEYKCKVSNKALPAPIEKTISKAKGQPRE |
| | | PQVYTLPPCRDELTKNQVSLWCLVKGFYPSDIAVEWESNGQ |
| | | PENNYKTTPPVLDSDGSFFLYSKLTVDKSRWQQGNVFSCSV |
| | | MHEALHNHYTQKSLSLSPGK |
| 74 | p35 (I41S)-15GS- | TPDPGMFPCLHHSQNLLRAVSNMLQKARQTLEFYPCTSEESD |
| | Fc (LALADS- | HEDITKDKTSTVEACLPLELTKNESCLNSRETSFITNGSCLAS |
| | YTE-KIH Knob: | RKTSFMMALCLSSIYEDLKMYQVEFKTMNAKLLMDPKRQIF |
| | S354C-T366W) | LDQNMLAVIDELMQALNFNSETVPQKSSLEEPDFYKTKIKLC |
| | | ILLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 75 | p35 (I41T)-15GS- | TPDPGMFPCLHHSQNLLRAVSNMLQKARQTLEFYPCTSEET |
| | Fc (LALADS- | DHEDITKDKTSTVEACLPLELTKNESCLNSRETSFITNGSCLA |
| | YTE-KIH Knob: | SRKTSFMMALCLSSIYEDLKMYQVEFKTMNAKLLMDPKRQI |
| | S354C-T366W) | FLDQNMLAVIDELMQALNFNSETVPQKSSLEEPDFYKTKIKL |
| | | CILLHAFRIRAVTIDRVMSYLNASGGGGSGGGGSGGGGSD |
| | | KTHTCPPCPAPEAAGGPSVFLFPPKPKDTLYITREPEVTCVVV |
| | | SVSHEDPEVKFNWYVDGVEVHNAKTKPREEQYNSTYRVVS |
| | | VLTVLHQDWLNGKEYKCKVSNKALPAPIEKTISKAKGQPRE |
| | | PQVYTLPPCRDELTKNQVSLWCLVKGFYPSDIAVEWESNGQ |
| | | PENNYKTTPPVLDSDGSFFLYSKLTVDKSRWQQGNVFSCSV |
| | | MHEALHNHYTQKSLSLSPGK |
| 76 | p35 (D42A)- | TPDPGMFPCLHHSQNLLRAVSNMLQKARQTLEFYPCTSEEIA |
| | 15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNFNSETVPQKSSLEEPDFYKTKIKLC |
| | S354C-T366W) | ILLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 77 | p35 (D42L)-15GS- | TPDPGMFPCLHHSQNLLRAVSNMLQKARQTLEFYPCTSEEIL |
| | Fc (LALADS- | HEDITKDKTSTVEACLPLELTKNESCLNSRETSFITNGSCLAS |
| | YTE-KIH Knob: | RKTSFMMALCLSSIYEDLKMYQVEFKTMNAKLLMDPKRQIF |
| | S354C-T366W) | LDQNMLAVIDELMQALNFNSETVPQKSSLEEPDFYKTKIKLC |
| | | ILLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 78 | p35 (D42W)- | TPDPGMFPCLHHSQNLLRAVSNMLQKARQTLEFYPCTSEEIW |
| | 15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNFNSETVPQKSSLEEPDFYKTKIKLC |
| | S354C-T366W) | ILLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 79 | p35 (E44A)-15GS- | TPDPGMFPCLHHSQNLLRAVSNMLQKARQTLEFYPCTSEEID |
| | Fc (LALADS- | HADITKDKTSTVEACLPLELTKNESCLNSRETSFITNGSCLAS |
| | YTE-KIH Knob: | RKTSFMMALCLSSIYEDLKMYQVEFKTMNAKLLMDPKRQIF |
| | S354C-T366W) | LDQNMLAVIDELMQALNFNSETVPQKSSLEEPDFYKTKIKLC |
| | | ILLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 80 | p35 (E44K)-15GS- | TPDPGMFPCLHHSQNLLRAVSNMLQKARQTLEFYPCTSEEID |
| | Fc (LALADS- | HKDITKDKTSTVEACLPLELTKNESCLNSRETSFITNGSCLAS |
| | YTE-KIH Knob: | RKTSFMMALCLSSIYEDLKMYQVEFKTMNAKLLMDPKRQIF |
| | S354C-T366W) | LDQNMLAVIDELMQALNFNSETVPQKSSLEEPDFYKTKIKLC |
| | | ILLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 81 | p35 (E44R)-15GS- | TPDPGMFPCLHHSQNLLRAVSNMLQKARQTLEFYPCTSEEID |
| | Fc (LALADS- | HRDITKDKTSTVEACLPLELTKNESCLNSRETSFITNGSCLAS |
| | YTE-KIH Knob: | RKTSFMMALCLSSIYEDLKMYQVEFKTMNAKLLMDPKRQIF |
| | S354C-T366W) | LDQNMLAVIDELMQALNFNSETVPQKSSLEEPDFYKTKIKLC |
| | | ILLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 82 | p35 (D159K)- | TPDPGMFPCLHHSQNLLRAVSNMLQKARQTLEFYPCTSEEID |
| | 15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNFNSETVPQKSSLEEPKFYKTKIKLC |
| | S354C-T366W) | ILLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 83 | p35 (D159R)- | TPDPGMFPCLHHSQNLLRAVSNMLQKARQTLEFYPCTSEEID |
| | 15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNFNSETVPQKSSLEEPRFYKTKIKLC |
| | S354C-T366W) | ILLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 84 | p35 (D159Y)- | TPDPGMFPCLHHSQNLLRAVSNMLQKARQTLEFYPCTSEEID |
| | 15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNFNSETVPQKSSLEEPYFYKTKIKLC |
| | S354C-T366W) | ILLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 85 | p35 (D159F)- | TPDPGMFPCLHHSQNLLRAVSNMLQKARQTLEFYPCTSEEID |
| | 15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNENSETVPQKSSLEEPFFYKTKIKLCI |
| | S354C-T366W) | LLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 86 | p35 (Y161S)- | TPDPGMFPCLHHSQNLLRAVSNMLQKARQTLEFYPCTSEEID |
| | 15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNFNSETVPQKSSLEEPDFSKTKIKLCI |
| | S354C-T366W) | LLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 87 | p35 (Y161T)- | TPDPGMFPCLHHSQNLLRAVSNMLQKARQTLEFYPCTSEEID |
| | 15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNFNSETVPQKSSLEEPDFTKTKIKLCI |
| | S354C-T366W) | LLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 88 | p35 (Y161A)- | TPDPGMFPCLHHSQNLLRAVSNMLQKARQTLEFYPCTSEEID |
| | 15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNFNSETVPQKSSLEEPDFAKTKIKLC |
| | S354C-T366W) | ILLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 89 | p35(Y161W)- | TPDPGMFPCLHHSQNLLRAVSNMLQKARQTLEFYPCTSEEID |
| | 15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNFNSETVPQKSSLEEPDFWKTKIKLC |
| | S354C-T366W) | ILLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 90 | p35 (K162E)- | TPDPGMFPCLHHSQNLLRAVSNMLQKARQTLEFYPCTSEEID |
| | 15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNFNSETVPQKSSLEEPDFYETKIKLCI |
| | S354C-T366W) | LLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 91 | p35 (K162D)- | TPDPGMFPCLHHSQNLLRAVSNMLQKARQTLEFYPCTSEEID |
| | 15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNFNSETVPQKSSLEEPDFYDTKIKLC |
| | S354C-T366W) | ILLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 92 | p40 (K85E)-4G-Fc | IWELKKDVYVVELDWYPDAPGEMVVLTCDTPEEDGITWTL |
| | (LALADS-YTE- | DQSSEVLGSGKTLTIQVKEFGDAGQYTCHKGGEVLSHSLLLL |
| | KIH Hole: Y349C- | HKEEDGIWSTDILKDQKEPKNKTFLRCEAKNYSGRFTCWWL |
| | T366S-L368A- | TTISTDLTFSVKSSRGSSDPQGVTCGAATLSAERVRGDNKEY |
| | Y407V) | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLYITREPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 93 | p40 (K85Y)-4G- | IWELKKDVYVVELDWYPDAPGEMVVLTCDTPEEDGITWTL |
| | Fc (LALADS- | DQSSEVLGSGKTLTIQVKEFGDAGQYTCHKGGEVLSHSLLLL |
| | YTE-KIH Hole: | HKYEDGIWSTDILKDQKEPKNKTFLRCEAKNYSGRFTCWWL |
| | Y349C-T366S- | TTISTDLTFSVKSSRGSSDPQGVTCGAATLSAERVRGDNKEY |
| | L368A-Y407V) | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLYITREPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 94 | p40 (D87A)-4G- | IWELKKDVYVVELDWYPDAPGEMVVLTCDTPEEDGITWTL |
| | Fc (LALADS- | DQSSEVLGSGKTLTIQVKEFGDAGQYTCHKGGEVLSHSLLLL |
| | YTE-KIH Hole: | HKKEAGIWSTDILKDQKEPKNKTFLRCEAKNYSGRFTCWWL |
| | Y349C-T366S- | TTISTDLTFSVKSSRGSSDPQGVTCGAATLSAERVRGDNKEY |
| | L368A-Y407V) | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLYITREPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 95 | p40 (D87L)-4G-Fc | IWELKKDVYVVELDWYPDAPGEMVVLTCDTPEEDGITWTL |
| | (LALADS-YTE- | DQSSEVLGSGKTLTIQVKEFGDAGQYTCHKGGEVLSHSLLLL |
| | KIH Hole: Y349C- | HKKELGIWSTDILKDQKEPKNKTFLRCEAKNYSGRFTCWWL |
| | T366S-L368A- | TTISTDLTFSVKSSRGSSDPQGVTCGAATLSAERVRGDNKEY |
| | Y407V) | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLYITREPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 96 | p40 (I89A)-4G-Fc | IWELKKDVYVVELDWYPDAPGEMVVLTCDTPEEDGITWTL |
| | (LALADS-YTE- | DQSSEVLGSGKTLTIQVKEFGDAGQYTCHKGGEVLSHSLLLL |
| | KIH Hole: Y349C- | HKKEDGAWSTDILKDQKEPKNKTFLRCEAKNYSGRFTCWW |
| | T366S-L368A- | LTTISTDLTFSVKSSRGSSDPQGVTCGAATLSAERVRGDNKE |
| | Y407V) | YEYSVECQEDSACPAAEESLPIEVMVDAVHKLKYENYTSSFF |
| | | IRDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLYITREPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 97 | p40 (I89S)-4G-Fc | IWELKKDVYVVELDWYPDAPGEMVVLTCDTPEEDGITWTL |
| | (LALADS-YTE- | DQSSEVLGSGKTLTIQVKEFGDAGQYTCHKGGEVLSHSLLLL |
| | KIH Hole: Y349C- | HKKEDGSWSTDILKDQKEPKNKTFLRCEAKNYSGRFTCWW |
| | T366S-L368A- | LTTISTDLTFSVKSSRGSSDPQGVTCGAATLSAERVRGDNKE |
| | Y407V) | YEYSVECQEDSACPAAEESLPIEVMVDAVHKLKYENYTSSFF |
| | | IRDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLYITREPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 98 | p40 (I89D)-4G-Fc | IWELKKDVYVVELDWYPDAPGEMVVLTCDTPEEDGITWTL |
| | (LALADS-YTE- | DQSSEVLGSGKTLTIQVKEFGDAGQYTCHKGGEVLSHSLLLL |
| | KIH Hole: Y349C- | HKKEDGDWSTDILKDQKEPKNKTFLRCEAKNYSGRFTCWW |
| | T366S-L368A- | LTTISTDLTFSVKSSRGSSDPQGVTCGAATLSAERVRGDNKE |
| | Y407V) | YEYSVECQEDSACPAAEESLPIEVMVDAVHKLKYENYTSSFF |
| | | IRDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLYITREPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 99 | p40 (I89P)-4G-Fc | IWELKKDVYVVELDWYPDAPGEMVVLTCDTPEEDGITWTL |
| | (LALADS-YTE- | DQSSEVLGSGKTLTIQVKEFGDAGQYTCHKGGEVLSHSLLLL |
| | KIH Hole: Y349C- | HKKEDGPWSTDILKDQKEPKNKTFLRCEAKNYSGRFTCWW |
| | T366S-L368A- | LTTISTDLTFSVKSSRGSSDPQGVTCGAATLSAERVRGDNKE |
| | Y407V) | YEYSVECQEDSACPAAEESLPIEVMVDAVHKLKYENYTSSFF |
| | | IRDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLYITREPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 100 | p40 (K104E)-4G- | IWELKKDVYVVELDWYPDAPGEMVVLTCDTPEEDGITWTL |
| | Fc (LALADS- | DQSSEVLGSGKTLTIQVKEFGDAGQYTCHKGGEVLSHSLLLL |
| | YTE-KIH Hole: | HKKEDGIWSTDILKDQKEPKNETFLRCEAKNYSGRFTCWWL |
| | Y349C-T366S- | TTISTDLTFSVKSSRGSSDPQGVTCGAATLSAERVRGDNKEY |
| | L368A-Y407V) | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLMISRTPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 101 | p40 (K104D)-4G- | IWELKKDVYVVELDWYPDAPGEMVVLTCDTPEEDGITWTL |
| | Fc (LALADS- | DQSSEVLGSGKTLTIQVKEFGDAGQYTCHKGGEVLSHSLLLL |
| | YTE-KIH Hole: | HKKEDGIWSTDILKDQKEPKNDTFLRCEAKNYSGRFTCWWL |
| | Y349C-T366S- | TTISTDLTFSVKSSRGSSDPQGVTCGAATLSAERVRGDNKEY |
| | L368A-Y407V) | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLYITREPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 102 | p40 (K104W)-4G- | IWELKKDVYVVELDWYPDAPGEMVVLTCDTPEEDGITWTL |
| | Fc (LALADS- | DQSSEVLGSGKTLTIQVKEFGDAGQYTCHKGGEVLSHSLLLL |
| | YTE-KIH Hole: | HKKEDGIWSTDILKDQKEPKNWTFLRCEAKNYSGRFTCWW |
| | Y349C-T366S- | LTTISTDLTFSVKSSRGSSDPQGVTCGAATLSAERVRGDNKE |
| | L368A-Y407V) | YEYSVECQEDSACPAAEESLPIEVMVDAVHKLKYENYTSSFF |
| | | IRDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLYITREPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 103 | p40 (K104S)-4G- | IWELKKDVYVVELDWYPDAPGEMVVLTCDTPEEDGITWTL |
| | Fc (LALADS- | DQSSEVLGSGKTLTIQVKEFGDAGQYTCHKGGEVLSHSLLLL |
| | YTE-KIH Hole: | HKKEDGIWSTDILKDQKEPKNSTFLRCEAKNYSGRFTCWWL |
| | Y349C-T366S- | TTISTDLTFSVKSSRGSSDPQGVTCGAATLSAERVRGDNKEY |
| | L368A-Y407V) | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLYITREPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 104 | p35 (D159K- | TPDPGMFPCLHHSQNLLRAVSNMLQKARQTLEFYPCTSEEID |
| | Y161T)-15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNFNSETVPQKSSLEEPKFTKTKIKLCI |
| | S354C-T366W) | LLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 105 | p35 (F33G- | TPDPGMFPCLHHSQNLLRAVSNMLQKARQTLEGYPCTSEEID |
| | D159K-Y161T)- | HEDITKDKTSTVEACLPLELTKNESCLNSRETSFITNGSCLAS |
| | 15GS-Fc | RKTSFMMALCLSSIYEDLKMYQVEFKTMNAKLLMDPKRQIF |
| | (LALADS-YTE- | LDQNMLAVIDELMQALNFNSETVPQKSSLEEPKFTKTKIKLCI |
| | KIH Knob: | LLHAFRIRAVTIDRVMSYLNASGGGGSGGGGSGGGGSDKT |
| | S354C-T366W) | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 106 | p35 (F33K- | TPDPGMFPCLHHSQNLLRAVSNMLQKARQTLEKYPCTSEEID |
| | D159K-Y161T)- | HEDITKDKTSTVEACLPLELTKNESCLNSRETSFITNGSCLAS |
| | 15GS-Fc | RKTSFMMALCLSSIYEDLKMYQVEFKTMNAKLLMDPKRQIF |
| | (LALADS-YTE- | LDQNMLAVIDELMQALNFNSETVPQKSSLEEPKFTKTKIKLCI |
| | KIH Knob: | LLHAFRIRAVTIDRVMSYLNASGGGGSGGGGSGGGGSDKT |
| | S354C-T366W) | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 107 | p35 (F33S- | TPDPGMFPCLHHSQNLLRAVSNMLQKARQTLESYPCTSEEID |
| | D159K-Y161T)- | HEDITKDKTSTVEACLPLELTKNESCLNSRETSFITNGSCLAS |
| | 15GS-Fc | RKTSFMMALCLSSIYEDLKMYQVEFKTMNAKLLMDPKRQIF |
| | (LALADS-YTE- | LDQNMLAVIDELMQALNFNSETVPQKSSLEEPKFTKTKIKLCI |
| | KIH Knob: | LLHAFRIRAVTIDRVMSYLNASGGGGSGGGGSGGGGSDKT |
| | S354C-T366W) | HTCPPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 108 | p35 (Y34G- | TPDPGMFPCLHHSQNLLRAVSNMLQKARQTLEFGPCTSEEID |
| | D159K-Y161T)- | HEDITKDKTSTVEACLPLELTKNESCLNSRETSFITNGSCLAS |
| | 15GS-Fc | RKTSFMMALCLSSIYEDLKMYQVEFKTMNAKLLMDPKRQIF |
| | (LALADS-YTE- | LDQNMLAVIDELMQALNFNSETVPQKSSLEEPKFTKTKIKLCI |
| | KIH Knob: | LLHAFRIRAVTIDRVMSYLNASGGGGSGGGGSGGGGSDKT |
| | S354C-T366W) | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 109 | p35 (Y34S- | TPDPGMFPCLHHSQNLLRAVSNMLQKARQTLEFSPCTSEEID |
| | D159K-Y161T)- | HEDITKDKTSTVEACLPLELTKNESCLNSRETSFITNGSCLAS |
| | 15GS-Fc | RKTSFMMALCLSSIYEDLKMYQVEFKTMNAKLLMDPKRQIF |
| | (LALADS-YTE- | LDQNMLAVIDELMQALNFNSETVPQKSSLEEPKFTKTKIKLCI |
| | KIH Knob: | LLHAFRIRAVTIDRVMSYLNASGGGGSGGGGSGGGGSDKT |
| | S354C-T366W) | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 110 | p35 (Y34T- | TPDPGMFPCLHHSQNLLRAVSNMLQKARQTLEFTPCTSEEID |
| | D159K-Y161T)- | HEDITKDKTSTVEACLPLELTKNESCLNSRETSFITNGSCLAS |
| | 15GS-Fc | RKTSFMMALCLSSIYEDLKMYQVEFKTMNAKLLMDPKRQIF |
| | (LALADS-YTE- | LDQNMLAVIDELMQALNFNSETVPQKSSLEEPKFTKTKIKLCI |
| | KIH Knob: | LLHAFRIRAVTIDRVMSYLNASGGGGSGGGGSGGGGSDKT |
| | S354C-T366W) | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 111 | p35 (P35Q- | TPDPGMFPCLHHSQNLLRAVSNMLQKARQTLEFYQCTSEEID |
| | D159K-Y161T)- | HEDITKDKTSTVEACLPLELTKNESCLNSRETSFITNGSCLAS |
| | 15GS-Fc | RKTSFMMALCLSSIYEDLKMYQVEFKTMNAKLLMDPKRQIF |
| | (LALADS-YTE- | LDQNMLAVIDELMQALNFNSETVPQKSSLEEPKFTKTKIKLCI |
| | KIH Knob: | LLHAFRIRAVTIDRVMSYLNASGGGGSGGGGSGGGGSDKT |
| | S354C-T366W) | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 112 | p35 (P35E- | TPDPGMFPCLHHSQNLLRAVSNMLQKARQTLEFYECTSEEID |
| | D159K-Y161T)- | HEDITKDKTSTVEACLPLELTKNESCLNSRETSFITNGSCLAS |
| | 15GS-Fc | RKTSFMMALCLSSIYEDLKMYQVEFKTMNAKLLMDPKRQIF |
| | (LALADS-YTE- | LDQNMLAVIDELMQALNFNSETVPQKSSLEEPKFTKTKIKLCI |
| | KIH Knob: | LLHAFRIRAVTIDRVMSYLNASGGGGSGGGGSGGGGSDKT |
| | S354C-T366W) | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 113 | p35 (F33S-Y34T | TPDPGMFPCLHHSQNLLRAVSNMLQKARQTLESTPCTSEEID |
| | D159K-Y161T)- | HEDITKDKTSTVEACLPLELTKNESCLNSRETSFITNGSCLAS |
| | 15GS-Fc | RKTSFMMALCLSSIYEDLKMYQVEFKTMNAKLLMDPKRQIF |
| | (LALADS-YTE- | LDQNMLAVIDELMQALNFNSETVPQKSSLEEPKFTKTKIKLCI |
| | KIH Knob: | LLHAFRIRAVTIDRVMSYLNASGGGGSGGGGSGGGGSDKT |
| | S354C-T366W) | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 114 | p35 (F33S-Y34S | TPDPGMFPCLHHSQNLLRAVSNMLQKARQTLESSPCTSEEID |
| | D159K-Y161T)- | HEDITKDKTSTVEACLPLELTKNESCLNSRETSFITNGSCLAS |
| | 15GS-Fc | RKTSFMMALCLSSIYEDLKMYQVEFKTMNAKLLMDPKRQIF |
| | (LALADS-YTE- | LDQNMLAVIDELMQALNFNSETVPQKSSLEEPKFTKTKIKLCI |
| | KIH Knob: | LLHAFRIRAVTIDRVMSYLNASGGGGSGGGGSGGGGSDKT |
| | S354C-T366W) | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 115 | p35 (F33G- | TPDPGMFPCLHHSQNLLRAVSNMLQKARQTLEGYPCTSEEID |
| | Y161T)-15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNFNSETVPQKSSLEEPDFTKTKIKLCI |
| | S354C-T366W) | LLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 116 | p35 (F33K- | TPDPGMFPCLHHSQNLLRAVSNMLQKARQTLEKYPCTSEEID |
| | Y161T)-15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNFNSETVPQKSSLEEPDFTKTKIKLCI |
| | S354C-T366W) | LLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 117 | p35 (F33S-Y161T)- | TPDPGMFPCLHHSQNLLRAVSNMLQKARQTLESYPCTSEEID |
| | 15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNFNSETVPQKSSLEEPDFTKTKIKLCI |
| | S354C-T366W) | LLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 118 | p35 (Y34G- | TPDPGMFPCLHHSQNLLRAVSNMLQKARQTLEFGPCTSEEID |
| | Y161T)-15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNFNSETVPQKSSLEEPDFTKTKIKLCI |
| | S354C-T366W) | LLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 119 | p35 (Y34S- | TPDPGMFPCLHHSQNLLRAVSNMLQKARQTLEFSPCTSEEID |
| | Y161T)-15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNFNSETVPQKSSLEEPDFTKTKIKLCI |
| | S354C-T366W) | LLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 120 | p35 (Y34T- | TPDPGMFPCLHHSQNLLRAVSNMLQKARQTLEFTPCTSEEID |
| | Y161T)-15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNFNSETVPQKSSLEEPDFTKTKIKLCI |
| | S354C-T366W) | LLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 121 | p35 (F33S-Y34T- | TPDPGMFPCLHHSQNLLRAVSNMLQKARQTLESTPCTSEEID |
| | Y161T)-15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNFNSETVPQKSSLEEPDFTKTKIKLCI |
| | S354C-T366W) | LLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 122 | p35 (F33S-Y34S- | TPDPGMFPCLHHSQNLLRAVSNMLQKARQTLESSPCTSEEID |
| | Y161T)-15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNFNSETVPQKSSLEEPDFTKTKIKLCI |
| | S354C-T366W) | LLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 123 | p35 (F33S-Y34G- | TPDPGMFPCLHHSQNLLRAVSNMLQKARQTLESGPCTSEEID |
| | Y161T)-15GS-Fc | HEDITKDKTSTVEACLPLELTKNESCLNSRETSFITNGSCLAS |
| | (LALADS-YTE- | RKTSFMMALCLSSIYEDLKMYQVEFKTMNAKLLMDPKRQIF |
| | KIH Knob: | LDQNMLAVIDELMQALNFNSETVPQKSSLEEPDFTKTKIKLCI |
| | S354C-T366W) | LLHAFRIRAVTIDRVMSYLNASGGGGSGGGGSGGGGSDKT |
| | | HTCPPCPAPEAAGGPSVFLFPPKPKDTLYITREPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREPQ |
| | | VYTLPPCRDELTKNQVSLWCLVKGFYPSDIAVEWESNGQPE |
| | | NNYKTTPPVLDSDGSFFLYSKLTVDKSRWQQGNVFSCSVMH |
| | | EALHNHYTQKSLSLSPGK |
| 124 | p40 (D87L- | IWELKKDVYVVELDWYPDAPGEMVVLTCDTPEEDGITWTL |
| | K104E)-4G-Fc | DQSSEVLGSGKTLTIQVKEFGDAGQYTCHKGGEVLSHSLLLL |
| | (LALADS-YTE- | HKKELGIWSTDILKDQKEPKNETFLRCEAKNYSGRFTCWWL |
| | KIH Hole: Y349C- | TTISTDLTFSVKSSRGSSDPQGVTCGAATLSAERVRGDNKEY |
| | T366S-L368A- | EYSVECQEDSACPAAEESLPIEVMVDAVHKLKYENYTSSFFI |
| | Y407V) | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLYITREPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 125 | p40 (D87L- | IWELKKDVYVVELDWYPDAPGEMVVLTCDTPEEDGITWTL |
| | K104D)-4G-Fc | DQSSEVLGSGKTLTIQVKEFGDAGQYTCHKGGEVLSHSLLLL |
| | (LALADS-YTE- | HKKELGIWSTDILKDQKEPKNDTFLRCEAKNYSGRFTCWWL |
| | KIH Hole: Y349C- | TTISTDLTFSVKSSRGSSDPQGVTCGAATLSAERVRGDNKEY |
| | T366S-L368A- | EYSVECQEDSACPAAEESLPIEVMVDAVHKLKYENYTSSFFI |
| | Y407V) | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLYITREPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 126 | p40 (P143A- | IWELKKDVYVVELDWYPDAPGEMVVLTCDTPEEDGITWTL |
| | P215A)-4G-Fc | DQSSEVLGSGKTLTIQVKEFGDAGQYTCHKGGEVLSHSLLLL |
| | (LALADS-YTE- | HKKEDGIWSTDILKDQKEPKNKTFLRCEAKNYSGRFTCWWL |
| | KIH Hole: Y349C- | TTISTDLTFSVKSSRGSSDAQGVTCGAATLSAERVRGDNKEY |
| | T366S-L368A- | EYSVECQEDSACPAAEESLPIEVMVDAVHKLKYENYTSSFFI |
| | Y407V) | RDIIKPDAPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCSGGGGDKTHTCPPCPAPEAAGGPSVF |
| | | LFPPKPKDTLYITREPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 127 | p40 (P143A- | IWELKKDVYVVELDWYPDAPGEMVVLTCDTPEEDGITWTL |
| | P215A-K260A- | DQSSEVLGSGKTLTIQVKEFGDAGQYTCHKGGEVLSHSLLLL |
| | R261S-V303I)- | HKKEDGIWSTDILKDQKEPKNKTFLRCEAKNYSGRFTCWWL |
| | 4G-Fc (LALADS- | TTISTDLTFSVKSSRGSSDAQGVTCGAATLSAERVRGDNKEY |
| | YTE-KIH Hole: | EYSVECQEDSACPAAEESLPIEVMVDAVHKLKYENYTSSFFI |
| | Y349C-T366S- | RDIIKPDAPKNLQLKPLKNSRQVEVSWEYPDTWSTPHSYFSL |
| | L368A-Y407V) | TFCVQVQGKSASEKKDRVFTDKTSATVICRKNASISVRAQDR |
| | | YYSSSWSEWASIPCSGGGGDKTHTCPPCPAPEAAGGPSVFLF |
| | | PPKPKDTLYITREPEVTCVVVSVSHEDPEVKFNWYVDGVEV |
| | | HNAKTKPREEQYNSTYRVVSVLTVLHQDWLNGKEYKCKVS |
| | | NKALPAPIEKTISKAKGQPREPQVCTLPPSRDELTKNQVSLSC |
| | | AVKGFYPSDIAVEWESNGQPENNYKTTPPVLDSDGSFFLVSK |
| | | LTVDKSRWQQGNVFSCSVMHEALHNHYTQKSLSLSPGK |
| 128 | p40 (P143A- | IWELKKDVYVVELDWYPDAPGEMVVLTCDTPEEDGITWTL |
| | P215A-K260S- | DQSSEVLGSGKTLTIQVKEFGDAGQYTCHKGGEVLSHSLLLL |
| | R261S-V303I)- | HKKEDGIWSTDILKDQKEPKNKTFLRCEAKNYSGRFTCWWL |
| | 4G-Fc (LALADS- | TTISTDLTFSVKSSRGSSDAQGVTCGAATLSAERVRGDNKEY |
| | YTE-KIH Hole: | EYSVECQEDSACPAAEESLPIEVMVDAVHKLKYENYTSSFFI |
| | Y349C-T366S- | RDIIKPDAPKNLQLKPLKNSRQVEVSWEYPDTWSTPHSYFSL |
| | L368A-Y407V) | TFCVQVQGKSSSEKKDRVFTDKTSATVICRKNASISVRAQDR |
| | | YYSSSWSEWASIPCSGGGGDKTHTCPPCPAPEAAGGPSVFLF |
| | | PPKPKDTLYITREPEVTCVVVSVSHEDPEVKFNWYVDGVEV |
| | | HNAKTKPREEQYNSTYRVVSVLTVLHQDWLNGKEYKCKVS |
| | | NKALPAPIEKTISKAKGQPREPQVCTLPPSRDELTKNQVSLSC |
| | | AVKGFYPSDIAVEWESNGQPENNYKTTPPVLDSDGSFFLVSK |
| | | LTVDKSRWQQGNVFSCSVMHEALHNHYTQKSLSLSPGK |
| 129 | p40 (K260A- | IWELKKDVYVVELDWYPDAPGEMVVLTCDTPEEDGITWTL |
| | R261S-V303I)- | DQSSEVLGSGKTLTIQVKEFGDAGQYTCHKGGEVLSHSLLLL |
| | 4G-Fc (LALADS- | HKKEDGIWSTDILKDQKEPKNKTFLRCEAKNYSGRFTCWWL |
| | YTE-KIH Hole: | TTISTDLTFSVKSSRGSSDPQGVTCGAATLSAERVRGDNKEY |
| | Y349C-T366S- | EYSVECQEDSACPAAEESLPIEVMVDAVHKLKYENYTSSFFI |
| | L368A-Y407V) | RDIIKPDPPKNLQLKPLKNSRQVEVSWEYPDTWSTPHSYFSL |
| | | TFCVQVQGKSASEKKDRVFTDKTSATVICRKNASISVRAQDR |
| | | YYSSSWSEWASIPCSGGGGDKTHTCPPCPAPEAAGGPSVFLF |
| | | PPKPKDTLYITREPEVTCVVVSVSHEDPEVKFNWYVDGVEV |
| | | HNAKTKPREEQYNSTYRVVSVLTVLHQDWLNGKEYKCKVS |
| | | NKALPAPIEKTISKAKGQPREPQVCTLPPSRDELTKNQVSLSC |
| | | AVKGFYPSDIAVEWESNGQPENNYKTTPPVLDSDGSFFLVSK |
| | | LTVDKSRWQQGNVFSCSVMHEALHNHYTQKSLSLSPGK |
| 130 | p40 (K260S- | IWELKKDVYVVELDWYPDAPGEMVVLTCDTPEEDGITWTL |
| | R261S-V303I)- | DQSSEVLGSGKTLTIQVKEFGDAGQYTCHKGGEVLSHSLLLL |
| | 4G-Fc (LALADS- | HKKEDGIWSTDILKDQKEPKNKTFLRCEAKNYSGRFTCWWL |
| | YTE-KIH Hole: | TTISTDLTFSVKSSRGSSDPQGVTCGAATLSAERVRGDNKEY |
| | Y349C-T366S- | EYSVECQEDSACPAAEESLPIEVMVDAVHKLKYENYTSSFFI |
| | L368A-Y407V) | RDIIKPDPPKNLQLKPLKNSRQVEVSWEYPDTWSTPHSYFSL |
| | | TFCVQVQGKSSSEKKDRVFTDKTSATVICRKNASISVRAQDR |
| | | YYSSSWSEWASIPCSGGGGDKTHTCPPCPAPEAAGGPSVFLF |
| | | PPKPKDTLYITREPEVTCVVVSVSHEDPEVKFNWYVDGVEV |
| | | HNAKTKPREEQYNSTYRVVSVLTVLHQDWLNGKEYKCKVS |
| | | NKALPAPIEKTISKAKGQPREPQVCTLPPSRDELTKNQVSLSC |
| | | AVKGFYPSDIAVEWESNGQPENNYKTTPPVLDSDGSFFLVSK |
| | | LTVDKSRWQQGNVFSCSVMHEALHNHYTQKSLSLSPGK |
| 131 | p40 (K260A- | IWELKKDVYVVELDWYPDAPGEMVVLTCDTPEEDGITWTL |
| | R261S)-4G-Fc | DQSSEVLGSGKTLTIQVKEFGDAGQYTCHKGGEVLSHSLLLL |
| | (LALADS-YTE- | HKKEDGIWSTDILKDQKEPKNKTFLRCEAKNYSGRFTCWWL |
| | KIH Hole: Y349C- | TTISTDLTFSVKSSRGSSDPQGVTCGAATLSAERVRGDNKEY |
| | T366S-L368A- | EYSVECQEDSACPAAEESLPIEVMVDAVHKLKYENYTSSFFI |
| | Y407V) | RDIIKPDPPKNLQLKPLKNSRQVEVSWEYPDTWSTPHSYFSL |
| | | TFCVQVQGKSASEKKDRVFTDKTSATVICRKNASISVRAQDR |
| | | YYSSSWSEWASVPCSGGGGDKTHTCPPCPAPEAAGGPSVFL |
| | | FPPKPKDTLYITREPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 132 | p40 (K260S- | IWELKKDVYVVELDWYPDAPGEMVVLTCDTPEEDGITWTL |
| | R261S)-4G-Fc | DQSSEVLGSGKTLTIQVKEFGDAGQYTCHKGGEVLSHSLLLL |
| | LALADS-YTE- | HKKEDGIWSTDILKDQKEPKNKTFLRCEAKNYSGRFTCWWL |
| | KIH Hole: Y349C- | TTISTDLTFSVKSSRGSSDPQGVTCGAATLSAERVRGDNKEY |
| | T366S-L368A- | EYSVECQEDSACPAAEESLPIEVMVDAVHKLKYENYTSSFFI |
| | Y407V) | RDIIKPDPPKNLQLKPLKNSRQVEVSWEYPDTWSTPHSYFSL |
| | | TFCVQVQGKSSSEKKDRVFTDKTSATVICRKNASISVRAQDR |
| | | YYSSSWSEWASVPCSGGGGDKTHTCPPCPAPEAAGGPSVFL |
| | | FPPKPKDTLYITREPEVTCVVVSVSHEDPEVKFNWYVDGVE |
| | | VHNAKTKPREEQYNSTYRVVSVLTVLHQDWLNGKEYKCKV |
| | | SNKALPAPIEKTISKAKGQPREPQVCTLPPSRDELTKNQVSLS |
| | | CAVKGFYPSDIAVEWESNGQPENNYKTTPPVLDSDGSFFLVS |
| | | KLTVDKSRWQQGNVFSCSVMHEALHNHYTQKSLSLSPGK |
| 133 | p35 (lacks N- | TPDPGMFPCLHHSQNLLRAVSNMLQKARQTLEFYPCTSEEID |
| | terminal | HEDITKDKTSTVEACLPLELTKNESCLNSRETSFITNGSCLAS |
| | hexapeptide | RKTSFMMALCLSSIYEDLKMYQVEFKTMNAKLLMDPKRQIF |
| | present in wild- | LDQNMLAVIDELMQALNFNSETVPQKSSLEEPDFYKTKIKLC |
| | type human IL- | ILLHAFRIRAVTIDRVMSYLNAS |
| | 12p35 subunit) | |
| 134 | P40 (mature wild- | IWELKKDVYVVELDWYPDAPGEMVVLTCDTPEEDGITWTL |
| | type Homo | DQSSEVLGSGKTLTIQVKEFGDAGQYTCHKGGEVLSHSLLLL |
| | sapiens) | HKKEDGIWSTDILKDQKEPKNKTFLRCEAKNYSGRFTCWWL |
| | | TTISTDLTFSVKSSRGSSDPQGVTCGAATLSAERVRGDNKEY |
| | | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCS |
| 135 | p35 (E40K) | TPDPGMFPCLHHSQNLLRAVSNMLQKARQTLEFYPCTSEKID |
| | | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFYKTKIKLC |
| | | ILLHAFRIRAVTIDRVMSYLNAS |
| 136 | p35 (E40R) | TPDPGMFPCLHHSQNLLRAVSNMLQKARQTLEFYPCTSERID |
| | | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFYKTKIKLC |
| | | ILLHAFRIRAVTIDRVMSYLNAS |
| 137 | p35 (E40W) | TPDPGMFPCLHHSQNLLRAVSNMLQKARQTLEFYPCTSEWI |
| | | DHEDITKDKTSTVEACLPLELTKNESCLNSRETSFITNGSCLA |
| | | SRKTSFMMALCLSSIYEDLKMYQVEFKTMNAKLLMDPKRQI |
| | | FLDQNMLAVIDELMQALNFNSETVPQKSSLEEPDFYKTKIKL |
| | | CILLHAFRIRAVTIDRVMSYLNAS |
| 138 | p35 (I41A) | TPDPGMFPCLHHSQNLLRAVSNMLQKARQTLEFYPCTSEEA |
| | | DHEDITKDKTSTVEACLPLELTKNESCLNSRETSFITNGSCLA |
| | | SRKTSFMMALCLSSIYEDLKMYQVEFKTMNAKLLMDPKRQI |
| | | FLDQNMLAVIDELMQALNFNSETVPQKSSLEEPDFYKTKIKL |
| | | CILLHAFRIRAVTIDRVMSYLNAS |
| 139 | p35 (I41V) | TPDPGMFPCLHHSQNLLRAVSNMLQKARQTLEFYPCTSEEV |
| | | DHEDITKDKTSTVEACLPLELTKNESCLNSRETSFITNGSCLA |
| | | SRKTSFMMALCLSSIYEDLKMYQVEFKTMNAKLLMDPKRQI |
| | | FLDQNMLAVIDELMQALNFNSETVPQKSSLEEPDFYKTKIKL |
| | | CILLHAFRIRAVTIDRVMSYLNAS |
| 140 | p35 (I41S) | TPDPGMFPCLHHSQNLLRAVSNMLQKARQTLEFYPCTSEESD |
| | | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFYKTKIKLC |
| | | ILLHAFRIRAVTIDRVMSYLNAS |
| 141 | p35 (I41T) | TPDPGMFPCLHHSQNLLRAVSNMLQKARQTLEFYPCTSEET |
| | | DHEDITKDKTSTVEACLPLELTKNESCLNSRETSFITNGSCLA |
| | | SRKTSFMMALCLSSIYEDLKMYQVEFKTMNAKLLMDPKRQI |
| | | FLDQNMLAVIDELMQALNFNSETVPQKSSLEEPDFYKTKIKL |
| | | CILLHAFRIRAVTIDRVMSYLNAS |
| 142 | p35 (D42A) | TPDPGMFPCLHHSQNLLRAVSNMLQKARQTLEFYPCTSEEIA |
| | | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFYKTKIKLC |
| | | ILLHAFRIRAVTIDRVMSYLNAS |
| 143 | p35 (D42L) | TPDPGMFPCLHHSQNLLRAVSNMLQKARQTLEFYPCTSEEIL |
| | | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFYKTKIKLC |
| | | ILLHAFRIRAVTIDRVMSYLNAS |
| 144 | p35 (D42W) | TPDPGMFPCLHHSQNLLRAVSNMLQKARQTLEFYPCTSEEIW |
| | | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFYKTKIKLC |
| | | ILLHAFRIRAVTIDRVMSYLNAS |
| 145 | p35 (E44A) | TPDPGMFPCLHHSQNLLRAVSNMLQKARQTLEFYPCTSEEID |
| | | HADITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFYKTKIKLC |
| | | ILLHAFRIRAVTIDRVMSYLNAS |
| 146 | p35 (E44K) | TPDPGMFPCLHHSQNLLRAVSNMLQKARQTLEFYPCTSEEID |
| | | HKDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFYKTKIKLC |
| | | ILLHAFRIRAVTIDRVMSYLNAS |
| 147 | p35 (E44R) | TPDPGMFPCLHHSQNLLRAVSNMLQKARQTLEFYPCTSEEID |
| | | HRDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFYKTKIKLC |
| | | ILLHAFRIRAVTIDRVMSYLNAS |
| 148 | p35 (D159K) | TPDPGMFPCLHHSQNLLRAVSNMLQKARQTLEFYPCTSEEID |
| | | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPKFYKTKIKLC |
| | | ILLHAFRIRAVTIDRVMSYLNAS |
| 149 | p35 (D159R) | TPDPGMFPCLHHSQNLLRAVSNMLQKARQTLEFYPCTSEEID |
| | | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPRFYKTKIKLC |
| | | ILLHAFRIRAVTIDRVMSYLNAS |
| 150 | p35 (D159Y) | TPDPGMFPCLHHSQNLLRAVSNMLQKARQTLEFYPCTSEEID |
| | | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPYFYKTKIKLC |
| | | ILLHAFRIRAVTIDRVMSYLNAS |
| 151 | p35 (D159F) | TPDPGMFPCLHHSQNLLRAVSNMLQKARQTLEFYPCTSEEID |
| | | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPFFYKTKIKLCI |
| | | LLHAFRIRAVTIDRVMSYLNAS |
| 152 | p35 (Y161S) | TPDPGMFPCLHHSQNLLRAVSNMLQKARQTLEFYPCTSEEID |
| | | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFSKTKIKLCI |
| | | LLHAFRIRAVTIDRVMSYLNAS |
| 153 | p35 (Y161T) | TPDPGMFPCLHHSQNLLRAVSNMLQKARQTLEFYPCTSEEID |
| | | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFTKTKIKLCI |
| | | LLHAFRIRAVTIDRVMSYLNAS |
| 154 | p35 (Y161A) | TPDPGMFPCLHHSQNLLRAVSNMLQKARQTLEFYPCTSEEID |
| | | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFAKTKIKLC |
| | | ILLHAFRIRAVTIDRVMSYLNAS |
| 155 | p35 (Y161W) | TPDPGMFPCLHHSQNLLRAVSNMLQKARQTLEFYPCTSEEID |
| | | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFWKTKIKLC |
| | | ILLHAFRIRAVTIDRVMSYLNAS |
| 156 | p35 (K162E) | TPDPGMFPCLHHSQNLLRAVSNMLQKARQTLEFYPCTSEEID |
| | | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFYETKIKLCI |
| | | LLHAFRIRAVTIDRVMSYLNAS |
| 157 | p35 (K162D) | TPDPGMFPCLHHSQNLLRAVSNMLQKARQTLEFYPCTSEEID |
| | | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFYDTKIKLC |
| | | ILLHAFRIRAVTIDRVMSYLNAS |
| 158 | p40 (K85E) | IWELKKDVYVVELDWYPDAPGEMVVLTCDTPEEDGITWTL |
| | | DQSSEVLGSGKTLTIQVKEFGDAGQYTCHKGGEVLSHSLLLL |
| | | HKEEDGIWSTDILKDQKEPKNKTFLRCEAKNYSGRFTCWWL |
| | | TTISTDLTFSVKSSRGSSDPQGVTCGAATLSAERVRGDNKEY |
| | | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCS |
| 159 | p40 (K85Y) | IWELKKDVYVVELDWYPDAPGEMVVLTCDTPEEDGITWTL |
| | | DQSSEVLGSGKTLTIQVKEFGDAGQYTCHKGGEVLSHSLLLL |
| | | HKYEDGIWSTDILKDQKEPKNKTFLRCEAKNYSGRFTCWWL |
| | | TTISTDLTFSVKSSRGSSDPQGVTCGAATLSAERVRGDNKEY |
| | | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCS |
| 160 | p40 (D87A) | IWELKKDVYVVELDWYPDAPGEMVVLTCDTPEEDGITWTL |
| | | DQSSEVLGSGKTLTIQVKEFGDAGQYTCHKGGEVLSHSLLLL |
| | | HKKEAGIWSTDILKDQKEPKNKTFLRCEAKNYSGRFTCWWL |
| | | TTISTDLTFSVKSSRGSSDPQGVTCGAATLSAERVRGDNKEY |
| | | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCS |
| 161 | p40 (D87L) | IWELKKDVYVVELDWYPDAPGEMVVLTCDTPEEDGITWTL |
| | | DQSSEVLGSGKTLTIQVKEFGDAGQYTCHKGGEVLSHSLLLL |
| | | HKKELGIWSTDILKDQKEPKNKTFLRCEAKNYSGRFTCWWL |
| | | TTISTDLTFSVKSSRGSSDPQGVTCGAATLSAERVRGDNKEY |
| | | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCS |
| 162 | p40 (I89A) | IWELKKDVYVVELDWYPDAPGEMVVLTCDTPEEDGITWTL |
| | | DQSSEVLGSGKTLTIQVKEFGDAGQYTCHKGGEVLSHSLLLL |
| | | HKKEDGAWSTDILKDQKEPKNKTFLRCEAKNYSGRFTCWW |
| | | LTTISTDLTFSVKSSRGSSDPQGVTCGAATLSAERVRGDNKE |
| | | YEYSVECQEDSACPAAEESLPIEVMVDAVHKLKYENYTSSFF |
| | | IRDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCS |
| 163 | p40 (I89S) | IWELKKDVYVVELDWYPDAPGEMVVLTCDTPEEDGITWTL |
| | | DQSSEVLGSGKTLTIQVKEFGDAGQYTCHKGGEVLSHSLLLL |
| | | HKKEDGSWSTDILKDQKEPKNKTFLRCEAKNYSGRFTCWW |
| | | LTTISTDLTFSVKSSRGSSDPQGVTCGAATLSAERVRGDNKE |
| | | YEYSVECQEDSACPAAEESLPIEVMVDAVHKLKYENYTSSFF |
| | | IRDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCS |
| 164 | p40 (I89D) | IWELKKDVYVVELDWYPDAPGEMVVLTCDTPEEDGITWTL |
| | | DQSSEVLGSGKTLTIQVKEFGDAGQYTCHKGGEVLSHSLLLL |
| | | HKKEDGDWSTDILKDQKEPKNKTFLRCEAKNYSGRFTCWW |
| | | LTTISTDLTFSVKSSRGSSDPQGVTCGAATLSAERVRGDNKE |
| | | YEYSVECQEDSACPAAEESLPIEVMVDAVHKLKYENYTSSFF |
| | | IRDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCS |
| 165 | p40 (I89P) | IWELKKDVYVVELDWYPDAPGEMVVLTCDTPEEDGITWTL |
| | | DQSSEVLGSGKTLTIQVKEFGDAGQYTCHKGGEVLSHSLLLL |
| | | HKKEDGPWSTDILKDQKEPKNKTFLRCEAKNYSGRFTCWW |
| | | LTTISTDLTFSVKSSRGSSDPQGVTCGAATLSAERVRGDNKE |
| | | YEYSVECQEDSACPAAEESLPIEVMVDAVHKLKYENYTSSFF |
| | | IRDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCS |
| 166 | p40 (K104E) | IWELKKDVYVVELDWYPDAPGEMVVLTCDTPEEDGITWTL |
| | | DQSSEVLGSGKTLTIQVKEFGDAGQYTCHKGGEVLSHSLLLL |
| | | HKKEDGIWSTDILKDQKEPKNETFLRCEAKNYSGRFTCWWL |
| | | TTISTDLTFSVKSSRGSSDPQGVTCGAATLSAERVRGDNKEY |
| | | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCS |
| 167 | p40 (K104D) | IWELKKDVYVVELDWYPDAPGEMVVLTCDTPEEDGITWTL |
| | | DQSSEVLGSGKTLTIQVKEFGDAGQYTCHKGGEVLSHSLLLL |
| | | HKKEDGIWSTDILKDQKEPKNDTFLRCEAKNYSGRFTCWWL |
| | | TTISTDLTFSVKSSRGSSDPQGVTCGAATLSAERVRGDNKEY |
| | | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCS |
| 168 | p40 (K104W) | IWELKKDVYVVELDWYPDAPGEMVVLTCDTPEEDGITWTL |
| | | DQSSEVLGSGKTLTIQVKEFGDAGQYTCHKGGEVLSHSLLLL |
| | | HKKEDGIWSTDILKDQKEPKNWTFLRCEAKNYSGRFTCWW |
| | | LTTISTDLTFSVKSSRGSSDPQGVTCGAATLSAERVRGDNKE |
| | | YEYSVECQEDSACPAAEESLPIEVMVDAVHKLKYENYTSSFF |
| | | IRDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCS |
| 169 | p40 (K104S) | IWELKKDVYVVELDWYPDAPGEMVVLTCDTPEEDGITWTL |
| | | DQSSEVLGSGKTLTIQVKEFGDAGQYTCHKGGEVLSHSLLLL |
| | | HKKEDGIWSTDILKDQKEPKNSTFLRCEAKNYSGRFTCWWL |
| | | TTISTDLTFSVKSSRGSSDPQGVTCGAATLSAERVRGDNKEY |
| | | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCS |
| 170 | p35 (D159K- | TPDPGMFPCLHHSQNLLRAVSNMLQKARQTLEFYPCTSEEID |
| | Y161T) | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPKFTKTKIKLCI |
| | | LLHAFRIRAVTIDRVMSYLNAS |
| 171 | p35 (F33G- | TPDPGMFPCLHHSQNLLRAVSNMLQKARQTLEGYPCTSEEID |
| | D159K-Y161T) | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPKFTKTKIKLCI |
| | | LLHAFRIRAVTIDRVMSYLNAS |
| 172 | p35 (F33K- | TPDPGMFPCLHHSQNLLRAVSNMLQKARQTLEKYPCTSEEID |
| | D159K-Y161T) | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPKFTKTKIKLCI |
| | | LLHAFRIRAVTIDRVMSYLNAS |
| 173 | p35 (F33S- | TPDPGMFPCLHHSQNLLRAVSNMLQKARQTLESYPCTSEEID |
| | D159K-Y161T) | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPKFTKTKIKLCI |
| | | LLHAFRIRAVTIDRVMSYLNAS |
| 174 | p35 (Y34G- | TPDPGMFPCLHHSQNLLRAVSNMLQKARQTLEFGPCTSEEID |
| | D159K-Y161T) | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPKFTKTKIKLCI |
| | | LLHAFRIRAVTIDRVMSYLNAS |
| 175 | p35 (Y34S- | TPDPGMFPCLHHSQNLLRAVSNMLQKARQTLEFSPCTSEEID |
| | D159K-Y161T) | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPKFTKTKIKLCI |
| | | LLHAFRIRAVTIDRVMSYLNAS |
| 176 | p35 (Y34T- | TPDPGMFPCLHHSQNLLRAVSNMLQKARQTLEFTPCTSEEID |
| | D159K-Y161T) | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPKFTKTKIKLCI |
| | | LLHAFRIRAVTIDRVMSYLNAS |
| 177 | p35 (P35Q- | TPDPGMFPCLHHSQNLLRAVSNMLQKARQTLEFYQCTSEEID |
| | D159K-Y161T) | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPKFTKTKIKLCI |
| | | LLHAFRIRAVTIDRVMSYLNAS |
| 178 | p35 (P35E- | TPDPGMFPCLHHSQNLLRAVSNMLQKARQTLEFYECTSEEID |
| | D159K-Y161T) | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPKFTKTKIKLCI |
| | | LLHAFRIRAVTIDRVMSYLNAS |
| 179 | p35 (F33S-Y34T | TPDPGMFPCLHHSQNLLRAVSNMLQKARQTLESTPCTSEEID |
| | D159K-Y161T) | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPKFTKTKIKLCI |
| | | LLHAFRIRAVTIDRVMSYLNAS |
| 180 | p35 (F33S-Y34S | TPDPGMFPCLHHSQNLLRAVSNMLQKARQTLESSPCTSEEID |
| | D159K-Y161T) | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPKFTKTKIKLCI |
| | | LLHAFRIRAVTIDRVMSYLNAS |
| 181 | p35 (F33G- | TPDPGMFPCLHHSQNLLRAVSNMLQKARQTLEGYPCTSEEID |
| | Y161T) | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFTKTKIKLCI |
| | | LLHAFRIRAVTIDRVMSYLNAS |
| 182 | p35 (F33K- | TPDPGMFPCLHHSQNLLRAVSNMLQKARQTLEKYPCTSEEID |
| | Y161T) | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFTKTKIKLCI |
| | | LLHAFRIRAVTIDRVMSYLNAS |
| 183 | p35 (F33S-Y161T) | TPDPGMFPCLHHSQNLLRAVSNMLQKARQTLESYPCTSEEID |
| | | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFTKTKIKLCI |
| | | LLHAFRIRAVTIDRVMSYLNAS |
| 184 | p35 (Y34G- | TPDPGMFPCLHHSQNLLRAVSNMLQKARQTLEFGPCTSEEID |
| | Y161T) | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFTKTKIKLCI |
| | | LLHAFRIRAVTIDRVMSYLNAS |
| 185 | p35 (Y34S- | TPDPGMFPCLHHSQNLLRAVSNMLQKARQTLEFSPCTSEEID |
| | Y161T) | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFTKTKIKLCI |
| | | LLHAFRIRAVTIDRVMSYLNAS |
| 186 | p35 (Y34T- | TPDPGMFPCLHHSQNLLRAVSNMLQKARQTLEFTPCTSEEID |
| | Y161T) | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFTKTKIKLCI |
| | | LLHAFRIRAVTIDRVMSYLNAS |
| 187 | p35 (F33S-Y34T- | TPDPGMFPCLHHSQNLLRAVSNMLQKARQTLESTPCTSEEID |
| | Y161T) | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFTKTKIKLCI |
| | | LLHAFRIRAVTIDRVMSYLNASG |
| 188 | p35 (F33S-Y34S- | TPDPGMFPCLHHSQNLLRAVSNMLQKARQTLESSPCTSEEID |
| | Y161T) | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFTKTKIKLCI |
| | | LLHAFRIRAVTIDRVMSYLNAS |
| 189 | p35 (F33S-Y34G- | TPDPGMFPCLHHSQNLLRAVSNMLQKARQTLESGPCTSEEID |
| | Y161T) | HEDITKDKTSTVEACLPLELTKNESCLNSRETSFITNGSCLAS |
| | | RKTSFMMALCLSSIYEDLKMYQVEFKTMNAKLLMDPKRQIF |
| | | LDQNMLAVIDELMQALNFNSETVPQKSSLEEPDFTKTKIKLCI |
| | | LLHAFRIRAVTIDRVMSYLNAS |
| 190 | p40 (D87L- | IWELKKDVYVVELDWYPDAPGEMVVLTCDTPEEDGITWTL |
| | K104E) | DQSSEVLGSGKTLTIQVKEFGDAGQYTCHKGGEVLSHSLLLL |
| | | HKKELGIWSTDILKDQKEPKNETFLRCEAKNYSGRFTCWWL |
| | | TTISTDLTFSVKSSRGSSDPQGVTCGAATLSAERVRGDNKEY |
| | | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCS |
| 191 | p40 (D87L- | IWELKKDVYVVELDWYPDAPGEMVVLTCDTPEEDGITWTL |
| | K104D) | DQSSEVLGSGKTLTIQVKEFGDAGQYTCHKGGEVLSHSLLLL |
| | | HKKELGIWSTDILKDQKEPKNDTFLRCEAKNYSGRFTCWWL |
| | | TTISTDLTFSVKSSRGSSDPQGVTCGAATLSAERVRGDNKEY |
| | | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCS |
| 192 | p40 (P143A- | IWELKKDVYVVELDWYPDAPGEMVVLTCDTPEEDGITWTL |
| | P215A) | DQSSEVLGSGKTLTIQVKEFGDAGQYTCHKGGEVLSHSLLLL |
| | | HKKEDGIWSTDILKDQKEPKNKTFLRCEAKNYSGRFTCWWL |
| | | TTISTDLTFSVKSSRGSSDAQGVTCGAATLSAERVRGDNKEY |
| | | EYSVECQEDSACPAAEESLPIEVMVDAVHKLKYENYTSSFFI |
| | | RDIIKPDAPKNLQLKPLKNSRQVEVSWEYPDTWSTPHSYFSL |
| | | TFCVQVQGKSKREKKDRVFTDKTSATVICRKNASISVRAQD |
| | | RYYSSSWSEWASVPCS |
| 193 | p40 (P143A- | IWELKKDVYVVELDWYPDAPGEMVVLTCDTPEEDGITWTL |
| | P215A-K260A- | DQSSEVLGSGKTLTIQVKEFGDAGQYTCHKGGEVLSHSLLLL |
| | R261S-V303I) | HKKEDGIWSTDILKDQKEPKNKTFLRCEAKNYSGRFTCWWL |
| | | TTISTDLTFSVKSSRGSSDAQGVTCGAATLSAERVRGDNKEY |
| | | EYSVECQEDSACPAAEESLPIEVMVDAVHKLKYENYTSSFFI |
| | | RDIIKPDAPKNLQLKPLKNSRQVEVSWEYPDTWSTPHSYFSL |
| | | TFCVQVQGKSASEKKDRVFTDKTSATVICRKNASISVRAQDR |
| | | YYSSSWSEWASIPCS |
| 194 | p40 (P143A- | IWELKKDVYVVELDWYPDAPGEMVVLTCDTPEEDGITWTL |
| | P215A-K260S- | DQSSEVLGSGKTLTIQVKEFGDAGQYTCHKGGEVLSHSLLLL |
| | R261S-V303I) | HKKEDGIWSTDILKDQKEPKNKTFLRCEAKNYSGRFTCWWL |
| | | TTISTDLTFSVKSSRGSSDAQGVTCGAATLSAERVRGDNKEY |
| | | EYSVECQEDSACPAAEESLPIEVMVDAVHKLKYENYTSSFFI |
| | | RDIIKPDAPKNLQLKPLKNSRQVEVSWEYPDTWSTPHSYFSL |
| | | TFCVQVQGKSSSEKKDRVFTDKTSATVICRKNASISVRAQDR |
| | | YYSSSWSEWASIPCS |
| 195 | p40 (K260A- | IWELKKDVYVVELDWYPDAPGEMVVLTCDTPEEDGITWTL |
| | R261S-V303I) | DQSSEVLGSGKTLTIQVKEFGDAGQYTCHKGGEVLSHSLLLL |
| | | HKKEDGIWSTDILKDQKEPKNKTFLRCEAKNYSGRFTCWWL |
| | | TTISTDLTFSVKSSRGSSDPQGVTCGAATLSAERVRGDNKEY |
| | | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSASEKKDRVFTDKTSATVICRKNASISVRAQDR |
| | | YYSSSWSEWASIPCS |
| 196 | p40 (K260S- | IWELKKDVYVVELDWYPDAPGEMVVLTCDTPEEDGITWTL |
| | R261S-V303I) | DQSSEVLGSGKTLTIQVKEFGDAGQYTCHKGGEVLSHSLLLL |
| | | HKKEDGIWSTDILKDQKEPKNKTFLRCEAKNYSGRFTCWWL |
| | | TTISTDLTFSVKSSRGSSDPQGVTCGAATLSAERVRGDNKEY |
| | | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSSSEKKDRVFTDKTSATVICRKNASISVRAQDR |
| | | YYSSSWSEWASIPCS |
| 197 | p40 (K260A- | IWELKKDVYVVELDWYPDAPGEMVVLTCDTPEEDGITWTL |
| | R261S) | DQSSEVLGSGKTLTIQVKEFGDAGQYTCHKGGEVLSHSLLLL |
| | | HKKEDGIWSTDILKDQKEPKNKTFLRCEAKNYSGRFTCWWL |
| | | TTISTDLTFSVKSSRGSSDPQGVTCGAATLSAERVRGDNKEY |
| | | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSASEKKDRVFTDKTSATVICRKNASISVRAQDR |
| | | YYSSSWSEWASVPCS |
| 198 | p40 (K260S- | IWELKKDVYVVELDWYPDAPGEMVVLTCDTPEEDGITWTL |
| | R261S) | DQSSEVLGSGKTLTIQVKEFGDAGQYTCHKGGEVLSHSLLLL |
| | | HKKEDGIWSTDILKDQKEPKNKTFLRCEAKNYSGRFTCWWL |
| | | TTISTDLTFSVKSSRGSSDPQGVTCGAATLSAERVRGDNKEY |
| | | EYSVECQEDSACPAAEESLPIEVMVDAVHKLKYENYTSSFFI |
| | | RDIIKPDPPKNLQLKPLKNSRQVEVSWEYPDTWSTPHSYFSL |
| | | TFCVQVQGKSSSEKKDRVFTDKTSATVICRKNASISVRAQDR |
| | | YYSSSWSEWASVPCS |
| 199 | Leader sequence A | MDPKGSLSWRILLFLSLAFELSYG |
| 200 | Leader peptide B | MSVPTQVLGLLLLWLTDARC |
| 201 | Leader peptide C | MEWSWVFLFFLSVTTGVHS |
| 202 | 4G | GGGG |
| 203 | (GGGGS)₂ | GGGGSGGGGS |
| 204 | G4S or GGGGS | GGGGS |
| 205 | (G4S)ₙ or | (G4S)n, wherein n is 1, 2, 3, 4, or 5 |
| | (GGGGS)ₙ, n is 1, | |
| | 2, 3, 4, or 5 | |
| 206 | (G4S)₃ or | GGGGSGGGGSGGGGS |
| | (GGGGS)₃ | |
| 207 | (G4S)ₙ or | (GGGGS)n, n is 1 to 10 peptide units |
| | (GGGGS)ₙ, n is 1 | |
| | to 10 peptide units | |
| 208 | Fc (LALADS-KIH | DKTHTCPPCPAPEAAGGPSVFLFPPKPKDTLMISRTPEVTCVV |
| | Knob: S354C- | VSVSHEDPEVKFNWYVDGVEVHNAKTKPREEQYNSTYRVV |
| | T366W) | SVLTVLHQDWLNGKEYKCKVSNKALPAPIEKTISKAKGQPR |
| | | EPQVYTLPPCRDELTKNQVSLWCLVKGFYPSDIAVEWESNG |
| | | QPENNYKTTPPVLDSDGSFFLYSKLTVDKSRWQQGNVFSCS |
| | | VMHEALHNHYTQKSLSLSPGK |
| 209 | Fc (LALADS-KIH | DKTHTCPPCPAPEAAGGPSVFLFPPKPKDTLMISRTPEVTCVV |
| | Hole: Y349C- | VSVSHEDPEVKFNWYVDGVEVHNAKTKPREEQYNSTYRVV |
| | T366S-L368A- | SVLTVLHQDWLNGKEYKCKVSNKALPAPIEKTISKAKGQPR |
| | Y407V) | EPQVCTLPPSRDELTKNQVSLSCAVKGFYPSDIAVEWESNGQ |
| | | PENNYKTTPPVLDSDGSFFLVSKLTVDKSRWQQGNVFSCSV |
| | | MHEALHNHYTQKSLSLSPGK |
| 210 | Fc (LALADS- | DKTHTCPPCPAPEAAGGPSVFLFPPKPKDTLYITREPEVTCVV |
| | YTE-KIH Knob: | VSVSHEDPEVKFNWYVDGVEVHNAKTKPREEQYNSTYRVV |
| | S354C-T366W) | SVLTVLHQDWLNGKEYKCKVSNKALPAPIEKTISKAKGQPR |
| | | EPQVYTLPPCRDELTKNQVSLWCLVKGFYPSDIAVEWESNG |
| | | QPENNYKTTPPVLDSDGSFFLYSKLTVDKSRWQQGNVFSCS |
| | | VMHEALHNHYTQKSLSLSPGK |
| 211 | Fc (LALADS- | DKTHTCPPCPAPEAAGGPSVFLFPPKPKDTLYITREPEVTCVV |
| | YTE-KIH Hole: | VSVSHEDPEVKFNWYVDGVEVHNAKTKPREEQYNSTYRVV |
| | Y349C-T366S- | SVLTVLHQDWLNGKEYKCKVSNKALPAPIEKTISKAKGQPR |
| | L368A-Y407V) | EPQVCTLPPSRDELTKNQVSLSCAVKGFYPSDIAVEWESNGQ |
| | | PENNYKTTPPVLDSDGSFFLVSKLTVDKSRWQQGNVFSCSV |
| | | MHEALHNHYTQKSLSLSPGK |
| 212 | Fc (LALADS-KIH | DKTHTCPPCPAPEAAGGPSVFLFPPKPKDTLMISRTPEVTCVV |
| | Knob: K409W) | VSVSHEDPEVKFNWYVDGVEVHNAKTKPREEQYNSTYRVV |
| | | SVLTVLHQDWLNGKEYKCKVSNKALPAPIEKTISKAKGQPR |
| | | EPQVYTLPPSRDELTKNQVSLTCLVKGFYPSDIAVEWESNGQ |
| | | PENNYKTTPPVLDSDGSFFLYSWLTVDKSRWQQGNVFSCSV |
| | | MHEALHNHYTQKSLSLSPGK |
| 213 | Fc (LALADS-KIH | DKTHTCPPCPAPEAAGGPSVFLFPPKPKDTLMISRTPEVTCVV |
| | Hole: D399V- | VSVSHEDPEVKFNWYVDGVEVHNAKTKPREEQYNSTYRVV |
| | F405T) | SVLTVLHQDWLNGKEYKCKVSNKALPAPIEKTISKAKGQPR |
| | | EPQVYTLPPSRDELTKNQVSLTCLVKGFYPSDIAVEWESNGQ |
| | | PENNYKTTPPVLVSDGSFTLYSKLTVDKSRWQQGNVFSCSV |
| | | MHEALHNHYTQKSLSLSPGK |
| 214 | Fc (LALADS- | DKTHTCPPCPAPEAAGGPSVFLFPPKPKDTLYITREPEVTCVV |
| | YTE-KIH Knob: | VSVSHEDPEVKFNWYVDGVEVHNAKTKPREEQYNSTYRVV |
| | K409W) | SVLTVLHQDWLNGKEYKCKVSNKALPAPIEKTISKAKGQPR |
| | | EPQVYTLPPSRDELTKNQVSLTCLVKGFYPSDIAVEWESNGQ |
| | | PENNYKTTPPVLDSDGSFFLYSWLTVDKSRWQQGNVFSCSV |
| | | MHEALHNHYTQKSLSLSPGK |
| 215 | Fc (LALADS- | DKTHTCPPCPAPEAAGGPSVFLFPPKPKDTLYITREPEVTCVV |
| | YTE-KIH Hole: | VSVSHEDPEVKFNWYVDGVEVHNAKTKPREEQYNSTYRVV |
| | D399V-F405T) | SVLTVLHQDWLNGKEYKCKVSNKALPAPIEKTISKAKGQPR |
| | | EPQVYTLPPSRDELTKNQVSLTCLVKGFYPSDIAVEWESNGQ |
| | | PENNYKTTPPVLVSDGSFTLYSKLTVDKSRWQQGNVFSCSV |
| | | MHEALHNHYTQKSLSLSPGK |
| 216 | Fc (LALADS-KIH | DKTHTCPPCPAPEAAGGPSVFLFPPKPKDTLMISRTPEVTCVV |
| | Knob: E357W) | VSVSHEDPEVKFNWYVDGVEVHNAKTKPREEQYNSTYRVV |
| | | SVLTVLHQDWLNGKEYKCKVSNKALPAPIEKTISKAKGQPR |
| | | EPQVYTLPPSRDWLTKNQVSLTCLVKGFYPSDIAVEWESNG |
| | | QPENNYKTTPPVLDSDGSFFLYSKLTVDKSRWQQGNVFSCS |
| | | VMHEALHNHYTQKSLSLSPGK |
| 217 | Fc (LALADS-KIH | DKTHTCPPCPAPEAAGGPSVFLFPPKPKDTLMISRTPEVTCVV |
| | Hole: Y349S) | VSVSHEDPEVKFNWYVDGVEVHNAKTKPREEQYNSTYRVV |
| | | SVLTVLHQDWLNGKEYKCKVSNKALPAPIEKTISKAKGQPR |
| | | EPQVSTLPPSRDELTKNQVSLTCLVKGFYPSDIAVEWESNGQ |
| | | PENNYKTTPPVLDSDGSFFLYSKLTVDKSRWQQGNVFSCSV |
| | | MHEALHNHYTQKSLSLSPGK |
| 218 | Fc (LALADS- | DKTHTCPPCPAPEAAGGPSVFLFPPKPKDTLYIYREPEVTCVV |
| | YTE-KIH Knob: | VSVSHEDPEVKFNWYVDGVEVHNAKTKPREEQYNSTYRVV |
| | E357W) | SVLTVLHQDWLNGKEYKCKVSNKALPAPIEKTISKAKGQPR |
| | | EPQVYTLPPSRDWLTKNQVSLTCLVKGFYPSDIAVEWESNG |
| | | QPENNYKTTPPVLDSDGSFFLYSKLTVDKSRWQQGNVFSCS |
| | | VMHEALHNHYTQKSLSLSPGK |
| 219 | Fc (LALADS- | DKTHTCPPCPAPEAAGGPSVFLFPPKPKDTLYITREPEVTCVV |
| | YTE-KIH Hole: | VSVSHEDPEVKFNWYVDGVEVHNAKTKPREEQYNSTYRVV |
| | Y349S) | SVLTVLHQDWLNGKEYKCKVSNKALPAPIEKTISKAKGQPR |
| | | EPQVSTLPPSRDELTKNQVSLTCLVKGFYPSDIAVEWESNGQ |
| | | PENNYKTTPPVLDSDGSFFLYSKLTVDKSRWQQGNVFSCSV |
| | | MHEALHNHYTQKSLSLSPGK |
| 220 | Fc (LALADS) | DKTHTCPPCPAPEAAGGPSVFLFPPKPKDTLMISRTPEVTCVV |
| | | VSVSHEDPEVKFNWYVDGVEVHNAKTKPREEQYNSTYRVV |
| | | SVLTVLHQDWLNGKEYKCKVSNKALPAPIEKTISKAKGQPR |
| | | EPQVYTLPPSRDELTKNQVSLTCLVKGFYPSDIAVEWESNGQ |
| | | PENNYKTTPPVLDSDGSFFLYSKLTVDKSRWQQGNVFSCSV |
| | | MHEALHNHYTQKSLSLSPGK |
| 221 | Fc (LALADS- | DKTHTCPPCPAPEAAGGPSVFLFPPKPKDTLYITREPEVTCVV |
| | YTE) | VSVSHEDPEVKFNWYVDGVEVHNAKTKPREEQYNSTYRVV |
| | | SVLTVLHQDWLNGKEYKCKVSNKALPAPIEKTISKAKGQPR |
| | | EPQVYTLPPSRDELTKNQVSLTCLVKGFYPSDIAVEWESNGQ |
| | | PENNYKTTPPVLDSDGSFFLYSKLTVDKSRWQQGNVFSCSV |
| | | MHEALHNHYTQKSLSLSPGK |
| 222 | Fc (Human IgG1) | DKTHTCPPCPAPELLGGPSVFLFPPKPKDTLMISRTPEVTCVV |
| | | VDVSHEDPEVKFNWYVDGVEVHNAKTKPREEQYNSTYRVV |
| | | SVLTVLHQDWLNGKEYKCKVSNKALPAPIEKTISKAKGQPR |
| | | EPQVYTLPPSRDELTKNQVSLTCLVKGFYPSDIAVEWESNGQ |
| | | PENNYKTTPPVLDSDGSFFLYSKLTVDKSRWQQGNVFSCSV |
| | | MHEALHNHYTQKSLSLSPGK |
| 223 | DNA encoding | ATGGACCCTAAGGGCTCTCTGAGCTGGAGGATTCTGCT |
| | IL-12p35 (I41S)- | GTTTCTGAGCCTCGCCTTTGAGCTGAGCTACGGCACTC |
| | 15GS-Fc | CAGATCCCGGCATGTTCCCTTGTCTGCACCACAGCCAGAA |
| | (LALADS-KIH | TCTGCTGAGGGCTGTGAGCAATATGCTGCAGAAGGCCAGA |
| | Knob: S354C- | CAGACACTCGAGTTCTACCCTTGCACAAGCGAGGAGAGCG |
| | T366W) | ACCACGAAGACATCACAAAGGATAAGACTAGCACTGTCG |
| | Features: | AGGCTTGTCTCCCTCTGGAGCTGACAAAGAACGAGAGCTG |
| | 1-72 encodes | CCTCAACTCTAGGGAGACAAGCTTCATCACTAACGGCAGC |
| | Leader A | TGTCTGGCTTCCAGAAAGACTAGCTTCATGATGGCTCTGT |
| | 73-645 encodes | GTCTGAGCAGCATCTACGAGGATCTGAAGATGTACCAAGT |
| | IL-12p35 | GGAGTTCAAAACTATGAACGCCAAGCTGCTCATGGACCCT |
| | 646-690 encodes | AAGAGGCAGATCTTTCTGGACCAGAACATGCTGGCCGTCA |
| | 15GS linker | TCGACGAGCTGATGCAAGCCCTCAATTTCAACAGCGAGAC |
| | 691-1374 encodes | TGTCCCTCAGAAGTCCTCTCTGGAGGAGCCAGACTTCTAT |
| | LALADS-KIH | AAGACTAAAATCAAACTCTGTATTCTGCTGCACGCCTTCA |
| | Knob: S354C- | GAATCAGAGCCGTGACAATCGATAGGGTGATGAGCTATCT |
| | T366W | GAATGCTAGCGGCGGAGGAGGCAGCGGAGGAGGCGGA |
| | | AGCGGCGGAGGCGGCTCCGACAAGACTCACACATGTCC |
| | | ACCTTGTCCAGCCCCAGAGGCTGCTGGAGGACCTTCCGTC |
| | | TTTCTGTTCCCACCTAAGCCTAAGGACACACTCATGATCTC |
| | | TAGGACACCAGAGGTGACATGTGTGGTCGTGTCCGTCAGC |
| | | CACGAGGACCCAGAGGTGAAGTTTAACTGGTACGTGGAC |
| | | GGCGTGGAAGTGCACAATGCCAAGACAAAGCCAAGGGAG |
| | | GAGCAATACAACAGCACTTATAGGGTCGTGAGCGTGCTGA |
| | | CAGTGCTGCATCAAGATTGGCTGAACGGCAAGGAGTACA |
| | | AGTGCAAGGTGAGCAACAAAGCTCTGCCAGCCCCTATCGA |
| | | GAAGACTATCAGCAAGGCCAAGGGCCAGCCTAGGGAGCC |
| | | ACAAGTGTACACACTGCCTCCTTGTAGGGACGAGCTGACT |
| | | AAGAACCAAGTGTCTCTGTGGTGTCTGGTCAAGGGCTTTT |
| | | ACCCTTCCGATATCGCCGTGGAGTGGGAATCCAACGGCCA |
| | | GCCAGAGAATAACTACAAGACAACTCCTCCAGTGCTGGAC |
| | | TCCGATGGCTCCTTCTTCCTCTACAGCAAGCTGACTGTGGA |
| | | TAAGAGCAGATGGCAGCAAGGCAACGTGTTCAGCTGTAG |
| | | CGTGATGCATGAGGCTCTGCACAACCACTACACTCAGAAG |
| | | TCTCTGTCTCTGTCCCCCGGCAAATGA |
| 224 | DNA encoding | ATGGACCCTAAGGGCTCTCTGAGCTGGAGGATTCTGCT |
| | IL-12p35 (E44R)- | GTTTCTGAGCCTCGCCTTTGAGCTGAGCTACGGCACTC |
| | 15GS-Fc | CAGATCCCGGCATGTTCCCTTGTCTGCACCACAGCCAGAA |
| | (LALADS-KIH | TCTGCTGAGGGCTGTGAGCAATATGCTGCAGAAGGCCAGA |
| | Knob: S354C- | CAGACACTCGAGTTCTACCCTTGCACAAGCGAGGAGATCG |
| | T366W) | ACCACAGaGACATCACAAAGGATAAGACTAGCACTGTCGA |
| | Features: | GGCTTGTCTCCCTCTGGAGCTGACAAAGAACGAGAGCTGC |
| | 1-72 encodes | CTCAACTCTAGGGAGACAAGCTTCATCACTAACGGCAGCT |
| | Leader A | GTCTGGCTTCCAGAAAGACTAGCTTCATGATGGCTCTGTG |
| | 73-645 encodes | TCTGAGCAGCATCTACGAGGATCTGAAGATGTACCAAGTG |
| | IL-12p35 | GAGTTCAAAACTATGAACGCCAAGCTGCTCATGGACCCTA |
| | 646-690 encodes | AGAGGCAGATCTTTCTGGACCAGAACATGCTGGCCGTCAT |
| | 15GS linker | CGACGAGCTGATGCAAGCCCTCAATTTCAACAGCGAGACT |
| | 691-1374 encodes | GTCCCTCAGAAGTCCTCTCTGGAGGAGCCAGACTTCTATA |
| | LALADS-KIH | AGACTAAAATCAAACTCTGTATTCTGCTGCACGCCTTCAG |
| | Knob: S354C- | AATCAGAGCCGTGACAATCGATAGGGTGATGAGCTATCTG |
| | T366W | AATGCTAGCGGCGGAGGAGGCAGCGGAGGAGGCGGAA |
| | | GCGGCGGAGGCGGCTCCGACAAGACTCACACATGTCCA |
| | | CCTTGTCCAGCCCCAGAGGCTGCTGGAGGACCTTCCGTCT |
| | | TTCTGTTCCCACCTAAGCCTAAGGACACACTCATGATCTCT |
| | | AGGACACCAGAGGTGACATGTGTGGTCGTGTCCGTCAGCC |
| | | ACGAGGACCCAGAGGTGAAGTTTAACTGGTACGTGGACG |
| | | GCGTGGAAGTGCACAATGCCAAGACAAAGCCAAGGGAGG |
| | | AGCAATACAACAGCACTTATAGGGTCGTGAGCGTGCTGAC |
| | | AGTGCTGCATCAAGATTGGCTGAACGGCAAGGAGTACAA |
| | | GTGCAAGGTGAGCAACAAAGCTCTGCCAGCCCCTATCGAG |
| | | AAGACTATCAGCAAGGCCAAGGGCCAGCCTAGGGAGCCA |
| | | CAAGTGTACACACTGCCTCCTTGTAGGGACGAGCTGACTA |
| | | AGAACCAAGTGTCTCTGTGGTGTCTGGTCAAGGGCTTTTA |
| | | CCCTTCCGATATCGCCGTGGAGTGGGAATCCAACGGCCAG |
| | | CCAGAGAATAACTACAAGACAACTCCTCCAGTGCTGGACT |
| | | CCGATGGCTCCTTCTTCCTCTACAGCAAGCTGACTGTGGAT |
| | | AAGAGCAGATGGCAGCAAGGCAACGTGTTCAGCTGTAGC |
| | | GTGATGCATGAGGCTCTGCACAACCACTACACTCAGAAGT |
| | | CTCTGTCTCTGTCCCCCGGCAAATGA |
| 225 | DNA encoding | ATGGACCCTAAGGGCTCTCTGAGCTGGAGGATTCTGCT |
| | IL-12p35 | GTTTCTGAGCCTCGCTTTTGAGCTCAGCTATGGCACTC |
| | (D159K)-15GS-Fc | CAGATCCCGGCATGTTCCCTTGTCTGCACCACAGCCAGAA |
| | (LALADS-KIH | TCTGCTGAGGGCCGTGAGCAATATGCTGCAGAAAGCTAGG |
| | Knob: S354C- | CAGACACTGGAGTTCTACCCTTGCACTAGCGAGGAGATCG |
| | T366W) | ATCACGAGGATATCACAAAGGACAAGACATCCACTGTGG |
| | Features: | AGGCTTGTCTGCCACTGGAGCTCACTAAGAACGAGAGCTG |
| | 1-72 encodes | TCTGAACTCTAGGGAAACAAGCTTCATCACTAATGGCAGC |
| | Leader A | TGTCTGGCTTCTAGGAAGACATCCTTCATGATGGCTCTGTG |
| | 73-645 encodes | TCTGAGCTCCATCTACGAGGATCTGAAGATGTACCAAGTG |
| | IL-12p35 | GAGTTCAAGACAATGAACGCCAAGCTGCTGATGGACCCTA |
| | 646-690 encodes | AGAGACAGATCTTTCTGGACCAGAACATGCTGGCCGTCAT |
| | 15GS linker | CGATGAACTGATGCAAGCTCTGAACTTCAACAGCGAGACA |
| | 691-1374 encodes | GTGCCTCAGAAGAGCTCTCTGGAGGAGCCAAAGTTCTACA |
| | LALADS-KIH | AGACAAAGATTAAACTGTGTATTCTGCTGCACGCCTTTAG |
| | Knob: S354C- | GATTAGGGCTGTCACTATCGATAGGGTGATGAGCTATCTG |
| | T366W | AATGCTAGCGGCGGAGGAGGCAGCGGAGGAGGAGGAA |
| | | GCGGAGGAGGCGGAAGCGACAAGACTCACACATGTCCA |
| | | CCTTGTCCAGCCCCAGAGGCTGCTGGAGGACCTTCCGTCT |
| | | TTCTGTTCCCACCTAAGCCTAAGGACACACTCATGATCTCT |
| | | AGGACACCAGAGGTGACATGTGTGGTCGTGTCCGTGAGCC |
| | | ACGAGGACCCAGAGGTGAAGTTCAACTGGTACGTGGACG |
| | | GCGTGGAAGTGCACAATGCCAAGACAAAGCCAAGGGAGG |
| | | AGCAATACAACAGCACTTATAGGGTGGTGAGCGTGCTGAC |
| | | AGTGCTGCATCAAGATTGGCTGAACGGCAAGGAGTACAA |
| | | ATGCAAGGTGAGCAATAAGGCTCTGCCAGCCCCTATCGAG |
| | | AAGACTATCTCCAAGGCTAAGGGCCAGCCTAGGGAGCCA |
| | | CAAGTGTACACTCTGCCTCCTTGTAGGGATGAGCTGACTA |
| | | AGAACCAAGTGTCTCTGTGGTGTCTGGTGAAAGGATTCTA |
| | | CCCTAGCGACATCGCCGTCGAGTGGGAGTCCAACGGCCAG |
| | | CCAGAGAACAACTACAAGACAACACCACCAGTGCTGGAC |
| | | TCCGATGGCTCCTTCTTTCTGTACAGCAAGCTGACTGTCGA |
| | | CAAGTCTAGGTGGCAGCAAGGCAACGTGTTCAGCTGTAGC |
| | | GTGATGCATGAGGCTCTGCACAATCACTACACACAGAAGT |
| | | CTCTGTCTCTGAGCCCCGGCAAATGA |
| 226 | DNA encoding | ATGGACCCTAAGGGCTCTCTGAGCTGGAGGATTCTGCT |
| | IL-12p35 | GTTTCTGAGCCTCGCTTTTGAGCTCAGCTATGGCACTC |
| | (D159R)-15GS-Fc | CAGATCCCGGCATGTTCCCTTGTCTGCACCACAGCCAGAA |
| | (LALADS-KIH | TCTGCTGAGGGCCGTGAGCAATATGCTGCAGAAAGCTAGG |
| | Knob: S354C- | CAGACACTGGAGTTCTACCCTTGCACTAGCGAGGAGATCG |
| | T366W) | ATCACGAGGATATCACAAAGGACAAGACATCCACTGTGG |
| | Features: | AGGCTTGTCTGCCACTGGAGCTCACTAAGAACGAGAGCTG |
| | 1-72 encodes | TCTGAACTCTAGGGAAACAAGCTTCATCACTAATGGCAGC |
| | Leader A | TGTCTGGCTTCTAGGAAGACATCCTTCATGATGGCTCTGTG |
| | 73-645 encodes | TCTGAGCTCCATCTACGAGGATCTGAAGATGTACCAAGTG |
| | IL-12p35 | GAGTTCAAGACAATGAACGCCAAGCTGCTGATGGACCCTA |
| | 646-690 encodes | AGAGACAGATCTTTCTGGACCAGAACATGCTGGCCGTCAT |
| | 15GS linker | CGATGAACTGATGCAAGCTCTGAACTTCAACAGCGAGACA |
| | 691-1374 encodes | GTGCCTCAGAAGAGCTCTCTGGAGGAGCCAAGGTTCTACA |
| | LALADS-KIH | AGACAAAGATTAAACTGTGTATTCTGCTGCACGCCTTTAG |
| | Knob: S354C- | GATTAGGGCTGTCACTATCGATAGGGTGATGAGCTATCTG |
| | T366W | AATGCTAGCGGCGGAGGAGGCAGCGGAGGAGGAGGAA |
| | | GCGGAGGAGGCGGAAGCGACAAGACTCACACATGTCCA |
| | | CCTTGTCCAGCCCCAGAGGCTGCTGGAGGACCTTCCGTCT |
| | | TTCTGTTCCCACCTAAGCCTAAGGACACACTCATGATCTCT |
| | | AGGACACCAGAGGTGACATGTGTGGTCGTGTCCGTGAGCC |
| | | ACGAGGACCCAGAGGTGAAGTTCAACTGGTACGTGGACG |
| | | GCGTGGAAGTGCACAATGCCAAGACAAAGCCAAGGGAGG |
| | | AGCAATACAACAGCACTTATAGGGTGGTGAGCGTGCTGAC |
| | | AGTGCTGCATCAAGATTGGCTGAACGGCAAGGAGTACAA |
| | | ATGCAAGGTGAGCAATAAGGCTCTGCCAGCCCCTATCGAG |
| | | AAGACTATCTCCAAGGCTAAGGGCCAGCCTAGGGAGCCA |
| | | CAAGTGTACACTCTGCCTCCTTGTAGGGATGAGCTGACTA |
| | | AGAACCAAGTGTCTCTGTGGTGTCTGGTGAAAGGATTCTA |
| | | CCCTAGCGACATCGCCGTCGAGTGGGAGTCCAACGGCCAG |
| | | CCAGAGAACAACTACAAGACAACACCACCAGTGCTGGAC |
| | | TCCGATGGCTCCTTCTTTCTGTACAGCAAGCTGACTGTCGA |
| | | CAAGTCTAGGTGGCAGCAAGGCAACGTGTTCAGCTGTAGC |
| | | GTGATGCATGAGGCTCTGCACAATCACTACACACAGAAGT |
| | | CTCTGTCTCTGAGCCCCGGCAAATGA |
| 227 | DNA encoding | ATGGACCCTAAGGGCTCTCTGAGCTGGAGGATTCTGCT |
| | IL-12p35 | GTTTCTGAGCCTCGCTTTTGAGCTCAGCTATGGCACTC |
| | (Y161S)-15GS-Fc | CAGATCCCGGCATGTTCCCTTGTCTGCACCACAGCCAGAA |
| | (LALADS-KIH | TCTGCTGAGGGCCGTGAGCAATATGCTGCAGAAAGCTAGG |
| | Knob: S354C- | CAGACACTGGAGTTCTACCCTTGCACTAGCGAGGAGATCG |
| | T366W) | ATCACGAGGATATCACAAAGGACAAGACATCCACTGTGG |
| | Features: | AGGCTTGTCTGCCACTGGAGCTCACTAAGAACGAGAGCTG |
| | 1-72 encodes | TCTGAACTCTAGGGAAACAAGCTTCATCACTAATGGCAGC |
| | Leader A | TGTCTGGCTTCTAGGAAGACATCCTTCATGATGGCTCTGTG |
| | 73-645 encodes | TCTGAGCTCCATCTACGAGGATCTGAAGATGTACCAAGTG |
| | IL-12p35 | GAGTTCAAGACAATGAACGCCAAGCTGCTGATGGACCCTA |
| | 646-690 encodes | AGAGACAGATCTTTCTGGACCAGAACATGCTGGCCGTCAT |
| | 15GS linker | CGATGAACTGATGCAAGCTCTGAACTTCAACAGCGAGACA |
| | 691-1374 encodes | GTGCCTCAGAAGAGCTCTCTGGAGGAGCCAGACTTCAGCA |
| | LALADS-KIH | AGACAAAGATTAAACTGTGTATTCTGCTGCACGCCTTTAG |
| | Knob: S354C- | GATTAGGGCTGTCACTATCGATAGGGTGATGAGCTATCTG |
| | T366W | AATGCTAGCGGCGGAGGAGGCAGCGGAGGAGGAGGAA |
| | | GCGGAGGAGGCGGAAGCGACAAGACTCACACATGTCCA |
| | | CCTTGTCCAGCCCCAGAGGCTGCTGGAGGACCTTCCGTCT |
| | | TTCTGTTCCCACCTAAGCCTAAGGACACACTCATGATCTCT |
| | | AGGACACCAGAGGTGACATGTGTGGTCGTGTCCGTGAGCC |
| | | ACGAGGACCCAGAGGTGAAGTTCAACTGGTACGTGGACG |
| | | GCGTGGAAGTGCACAATGCCAAGACAAAGCCAAGGGAGG |
| | | AGCAATACAACAGCACTTATAGGGTGGTGAGCGTGCTGAC |
| | | AGTGCTGCATCAAGATTGGCTGAACGGCAAGGAGTACAA |
| | | ATGCAAGGTGAGCAATAAGGCTCTGCCAGCCCCTATCGAG |
| | | AAGACTATCTCCAAGGCTAAGGGCCAGCCTAGGGAGCCA |
| | | CAAGTGTACACTCTGCCTCCTTGTAGGGATGAGCTGACTA |
| | | AGAACCAAGTGTCTCTGTGGTGTCTGGTGAAAGGATTCTA |
| | | CCCTAGCGACATCGCCGTCGAGTGGGAGTCCAACGGCCAG |
| | | CCAGAGAACAACTACAAGACAACACCACCAGTGCTGGAC |
| | | TCCGATGGCTCCTTCTTTCTGTACAGCAAGCTGACTGTCGA |
| | | CAAGTCTAGGTGGCAGCAAGGCAACGTGTTCAGCTGTAGC |
| | | GTGATGCATGAGGCTCTGCACAATCACTACACACAGAAGT |
| | | CTCTGTCTCTGAGCCCCGGCAAATGA |
| 228 | DNA encoding | ATGGACCCTAAGGGCTCTCTGAGCTGGAGGATTCTGCT |
| | IL-12p35 | GTTTCTGAGCCTCGCTTTTGAGCTCAGCTATGGCACTC |
| | (Y161T)-15GS-Fc | CAGATCCCGGCATGTTCCCTTGTCTGCACCACAGCCAGAA |
| | (LALADS-KIH | TCTGCTGAGGGCCGTGAGCAATATGCTGCAGAAAGCTAGG |
| | Knob: S354C- | CAGACACTGGAGTTCTACCCTTGCACTAGCGAGGAGATCG |
| | T366W) | ATCACGAGGATATCACAAAGGACAAGACATCCACTGTGG |
| | Features: | AGGCTTGTCTGCCACTGGAGCTCACTAAGAACGAGAGCTG |
| | 1-72 encodes | TCTGAACTCTAGGGAAACAAGCTTCATCACTAATGGCAGC |
| | Leader A | TGTCTGGCTTCTAGGAAGACATCCTTCATGATGGCTCTGTG |
| | 73-645 encodes | TCTGAGCTCCATCTACGAGGATCTGAAGATGTACCAAGTG |
| | IL-12p35 | GAGTTCAAGACAATGAACGCCAAGCTGCTGATGGACCCTA |
| | 646-690 encodes | AGAGACAGATCTTTCTGGACCAGAACATGCTGGCCGTCAT |
| | 15GS linker | CGATGAACTGATGCAAGCTCTGAACTTCAACAGCGAGACA |
| | 691-1374 encodes | GTGCCTCAGAAGAGCTCTCTGGAGGAGCCAGACTTCACTA |
| | LALADS-KIH | AGACAAAGATTAAACTGTGTATTCTGCTGCACGCCTTTAG |
| | Knob: S354C- | GATTAGGGCTGTCACTATCGATAGGGTGATGAGCTATCTG |
| | T366W | AATGCTAGCGGCGGAGGAGGCAGCGGAGGAGGAGGAA |
| | | GCGGAGGAGGCGGAAGCGACAAGACTCACACATGTCCA |
| | | CCTTGTCCAGCCCCAGAGGCTGCTGGAGGACCTTCCGTCT |
| | | TTCTGTTCCCACCTAAGCCTAAGGACACACTCATGATCTCT |
| | | AGGACACCAGAGGTGACATGTGTGGTCGTGTCCGTGAGCC |
| | | ACGAGGACCCAGAGGTGAAGTTCAACTGGTACGTGGACG |
| | | GCGTGGAAGTGCACAATGCCAAGACAAAGCCAAGGGAGG |
| | | AGCAATACAACAGCACTTATAGGGTGGTGAGCGTGCTGAC |
| | | AGTGCTGCATCAAGATTGGCTGAACGGCAAGGAGTACAA |
| | | ATGCAAGGTGAGCAATAAGGCTCTGCCAGCCCCTATCGAG |
| | | AAGACTATCTCCAAGGCTAAGGGCCAGCCTAGGGAGCCA |
| | | CAAGTGTACACTCTGCCTCCTTGTAGGGATGAGCTGACTA |
| | | AGAACCAAGTGTCTCTGTGGTGTCTGGTGAAAGGATTCTA |
| | | CCCTAGCGACATCGCCGTCGAGTGGGAGTCCAACGGCCAG |
| | | CCAGAGAACAACTACAAGACAACACCACCAGTGCTGGAC |
| | | TCCGATGGCTCCTTCTTTCTGTACAGCAAGCTGACTGTCGA |
| | | CAAGTCTAGGTGGCAGCAAGGCAACGTGTTCAGCTGTAGC |
| | | GTGATGCATGAGGCTCTGCACAATCACTACACACAGAAGT |
| | | CTCTGTCTCTGAGCCCCGGCAAATGA |
| 229 | DNA encoding | ATGGACCCTAAGGGCTCTCTGAGCTGGAGGATTCTGCT |
| | IL-12p35 | GTTTCTGAGCCTCGCTTTTGAGCTCAGCTATGGCACTC |
| | (Y161A)-15GS-Fc | CAGATCCCGGCATGTTCCCTTGTCTGCACCACAGCCAGAA |
| | (LALADS-KIH | TCTGCTGAGGGCCGTGAGCAATATGCTGCAGAAAGCTAGG |
| | Knob: S354C- | CAGACACTGGAGTTCTACCCTTGCACTAGCGAGGAGATCG |
| | T366W) | ATCACGAGGATATCACAAAGGACAAGACATCCACTGTGG |
| | Features: | AGGCTTGTCTGCCACTGGAGCTCACTAAGAACGAGAGCTG |
| | 1-72 encodes | TCTGAACTCTAGGGAAACAAGCTTCATCACTAATGGCAGC |
| | Leader A | TGTCTGGCTTCTAGGAAGACATCCTTCATGATGGCTCTGTG |
| | 73-645 encodes | TCTGAGCTCCATCTACGAGGATCTGAAGATGTACCAAGTG |
| | IL-12p35 | GAGTTCAAGACAATGAACGCCAAGCTGCTGATGGACCCTA |
| | 646-690 encodes | AGAGACAGATCTTTCTGGACCAGAACATGCTGGCCGTCAT |
| | 15GS linker | CGATGAACTGATGCAAGCTCTGAACTTCAACAGCGAGACA |
| | 691-1374 encodes | GTGCCTCAGAAGAGCTCTCTGGAGGAGCCAGACTTCGCTA |
| | LALADS-KIH | AGACAAAGATTAAACTGTGTATTCTGCTGCACGCCTTTAG |
| | Knob: S354C- | GATTAGGGCTGTCACTATCGATAGGGTGATGAGCTATCTG |
| | T366W | AATGCTAGCGGCGGAGGAGGCAGCGGAGGAGGAGGAA |
| | | GCGGAGGAGGCGGAAGCGACAAGACTCACACATGTCCA |
| | | CCTTGTCCAGCCCCAGAGGCTGCTGGAGGACCTTCCGTCT |
| | | TTCTGTTCCCACCTAAGCCTAAGGACACACTCATGATCTCT |
| | | AGGACACCAGAGGTGACATGTGTGGTCGTGTCCGTGAGCC |
| | | ACGAGGACCCAGAGGTGAAGTTCAACTGGTACGTGGACG |
| | | GCGTGGAAGTGCACAATGCCAAGACAAAGCCAAGGGAGG |
| | | AGCAATACAACAGCACTTATAGGGTGGTGAGCGTGCTGAC |
| | | AGTGCTGCATCAAGATTGGCTGAACGGCAAGGAGTACAA |
| | | ATGCAAGGTGAGCAATAAGGCTCTGCCAGCCCCTATCGAG |
| | | AAGACTATCTCCAAGGCTAAGGGCCAGCCTAGGGAGCCA |
| | | CAAGTGTACACTCTGCCTCCTTGTAGGGATGAGCTGACTA |
| | | AGAACCAAGTGTCTCTGTGGTGTCTGGTGAAAGGATTCTA |
| | | CCCTAGCGACATCGCCGTCGAGTGGGAGTCCAACGGCCAG |
| | | CCAGAGAACAACTACAAGACAACACCACCAGTGCTGGAC |
| | | TCCGATGGCTCCTTCTTTCTGTACAGCAAGCTGACTGTCGA |
| | | CAAGTCTAGGTGGCAGCAAGGCAACGTGTTCAGCTGTAGC |
| | | GTGATGCATGAGGCTCTGCACAATCACTACACACAGAAGT |
| | | CTCTGTCTCTGAGCCCCGGCAAATGA |
| 230 | DNA encoding | ATGGACCCTAAGGGCTCTCTGAGCTGGAGGATTCTGCT |
| | IL-12p40-4G-Fc | CTTTCTCTCTCTGGCCTTTGAACTGAGCTACGGAATCT |
| | (LALADS-KIH | GGGAGCTGAAGAAAGACGTCTACGTGGTCGAGCTGGACT |
| | Hole: Y349C- | GGTACCCAGATGCCCCCGGCGAGATGGTCGTCCTCACATG |
| | T366S-L368A- | CGACACACCAGAGGAAGACGGCATCACTTGGACTCTCGAT |
| | Y407V) | CAGTCCTCCGAAGTGCTGGGCTCCGGAAAGACACTGACAA |
| | Features: | TCCAAGTGAAGGAGTTCGGCGATGCTGGACAGTACACTTG |
| | 1-72 encode | CCACAAAGGAGGAGAGGTGCTGAGCCACTCTCTGCTGCTG |
| | Leader A | CTGCACAAGAAAGAGGACGGAATCTGGTCCACAGACATT |
| | 73-990 encodes | CTCAAGGACCAAAAGGAGCCTAAGAACAAGACATTTCTG |
| | IL-12p40 | AGGTGCGAGGCCAAGAACTACAGCGGCAGATTCACTTGCT |
| | 991-1002 encodes | GGTGGCTCACAACAATCAGCACAGACCTCACATTCTCCGT |
| | 4G linker | GAAGTCCTCCAGAGGCAGCTCCGACCCACAAGGCGTGACT |
| | 1003-1686 | TGTGGAGCTGCTACACTCAGCGCCGAAAGGGTGAGGGGA |
| | encodes | GACAATAAGGAGTACGAGTATAGCGTCGAGTGCCAAGAG |
| | (LALADS-KIH | GATAGCGCTTGTCCAGCTGCCGAAGAGTCTCTGCCAATCG |
| | Hole: Y349C- | AGGTCATGGTGGATGCCGTCCACAAGCTCAAATACGAGA |
| | T366S-L368A- | ATTACACTAGCTCCTTCTTCATTAGGGACATTATTAAGCCA |
| | Y407V) | GACCCTCCTAAGAACCTCCAGCTGAAGCCACTCAAGAACT |
| | | CTAGGCAAGTGGAGGTGAGCTGGGAGTATCCAGATACTTG |
| | | GTCCACACCTCACTCCTACTTCAGCCTCACATTCTGCGTCC |
| | | AAGTGCAAGGCAAGTCCAAGAGAGAGAAGAAGGATAGG |
| | | GTCTTCACAGATAAGACAAGCGCCACAGTGATCTGTAGGA |
| | | AGAACGCCTCCATCAGCGTGAGGGCCCAAGATAGGTACT |
| | | ATTCCTCCAGCTGGAGCGAGTGGGCTAGCGTCCCTTGCAG |
| | | CGGAGGAGGCGGAGACAAAACTCATACTTGCCCACCTTG |
| | | CCCAGCCCCAGAAGCTGCTGGCGGACCATCCGTGTTTCTG |
| | | TTCCCACCTAAGCCTAAGGACACTCTGATGATCTCTAGGA |
| | | CACCAGAGGTGACTTGTGTGGTGGTGAGCGTGAGCCATGA |
| | | AGATCCAGAGGTGAAGTTCAACTGGTACGTCGATGGCGTG |
| | | GAGGTGCATAACGCCAAGACAAAACCAAGGGAGGAACAG |
| | | TACAACAGCACATATAGGGTGGTCAGCGTCCTCACTGTGC |
| | | TCCACCAAGATTGGCTCAATGGCAAAGAATACAAGTGTAA |
| | | GGTCTCCAACAAAGCTCTGCCAGCTCCAATCGAGAAGACT |
| | | ATCTCCAAGGCCAAGGGCCAACCTAGGGAGCCACAAGTG |
| | | TGCACTCTGCCACCTTCTAGGGACGAACTGACTAAGAACC |
| | | AAGTGTCTCTGAGCTGCGCCGTGAAGGGATTTTACCCTTC |
| | | CGATATCGCCGTGGAGTGGGAGAGCAATGGCCAACCAGA |
| | | GAATAATTATAAGACAACTCCTCCAGTGCTGGACTCCGAC |
| | | GGCAGCTTCTTTCTGGTCAGCAAGCTGACTGTGGACAAGT |
| | | CTAGGTGGCAGCAAGGAAACGTGTTCAGCTGTTCCGTGAT |
| | | GCATGAAGCTCTGCACAACCACTACACACAGAAGAGCCTC |
| | | AGCCTCAGCCCCGGCAAGTGA |
| 231 | DNA encoding | ATGGACCCTAAGGGCAGCCTCAGCTGGAGGATTCTGC |
| | IL-12p40(D87L)- | TGTTTCTGTCTCTGGCCTTTGAGCTGAGCTATGGCATT |
| | 4G-Fc (LALADS- | TGGGAGCTGAAGAAGGACGTGTACGTGGTGGAGCTGGAT |
| | KIH Hole: Y349C- | TGGTATCCAGATGCCCCCGGCGAGATGGTGGTGCTGACAT |
| | T366S-L368A- | GTGACACTCCAGAGGAAGACGGCATCACTTGGACTCTCGA |
| | Y407V) | TCAGTCCTCCGAAGTGCTGGGCTCCGGAAAGACACTGACA |
| | Features: | ATCCAAGTGAAGGAGTTCGGAGATGCTGGCCAGTACACTT |
| | 1-72 encode | GCCACAAAGGCGGCGAAGTGCTGAGCCACTCTCTGCTGCT |
| | Leader A | GCTGCATAAGAAGGAGCTGGGAATCTGGTCCACAGACAT |
| | 73-990 encodes | CCTCAAGGACCAAAAGGAGCCAAAAAACAAGACATTTCT |
| | IL-12p40 | GAGGTGTGAGGCCAAGAACTACTCCGGAAGGTTTACATGC |
| | 991-1002 encodes | TGGTGGCTCACAACAATCTCCACAGACCTCACTTTCAGCG |
| | 4G linker | TGAAGAGCAGCAGAGGCAGCAGCGACCCACAAGGCGTGA |
| | 1003-1686 | CTTGTGGAGCCGCCACTCTGTCCGCTGAGAGGGTGAGGGG |
| | encodes | CGACAACAAGGAGTACGAGTACTCCGTGGAGTGCCAAGA |
| | (LALADS-KIH | GGATAGCGCTTGTCCAGCCGCTGAGGAAAGCCTCCCTATC |
| | Hole: Y349C- | GAGGTGATGGTGGACGCCGTGCACAAGCTGAAGTATGAA |
| | T366S-L368A- | AATTACACATCCAGCTTCTTCATTAGGGACATCATCAAGC |
| | Y407V) | CAGATCCTCCAAAGAACCTCCAGCTCAAGCCTCTGAAGAA |
| | | CTCTAGGCAAGTGGAGGTCAGCTGGGAGTATCCAGACACT |
| | | TGGAGCACACCTCACTCCTACTTCTCTCTGACATTCTGCGT |
| | | CCAAGTGCAAGGCAAGTCCAAGAGAGAGAAAAAGGATAG |
| | | GGTGTTCACTGACAAGACTAGCGCCACAGTGATCTGCAGA |
| | | AAGAACGCCAGCATCAGCGTGAGAGCCCAAGATAGGTAC |
| | | TATTCCTCCAGCTGGAGCGAGTGGGCTTCCGTGCCTTGTA |
| | | GCGGCGGAGGAGGCGACAAGACTCACACTTGTCCACCAT |
| | | GCCCAGCCCCAGAAGCTGCCGGAGGCCCTAGCGTGTTTCT |
| | | GTTCCCTCCAAAGCCTAAGGACACACTGATGATCTCTAGG |
| | | ACTCCAGAAGTCACATGTGTCGTGGTGTCCGTCAGCCACG |
| | | AAGATCCAGAGGTCAAGTTCAACTGGTACGTGGATGGAGT |
| | | CGAGGTGCACAATGCCAAGACAAAGCCAAGGGAGGAGCA |
| | | GTACAACAGCACATATAGGGTGGTCAGCGTGCTGACTGTG |
| | | CTCCACCAAGATTGGCTGAACGGCAAGGAGTACAAGTGC |
| | | AAGGTCAGCAACAAGGCTCTGCCAGCTCCAATCGAGAAG |
| | | ACTATCTCCAAGGCCAAGGGCCAGCCAAGGGAGCCACAA |
| | | GTGTGCACACTGCCTCCATCTAGGGACGAACTGACAAAGA |
| | | ACCAAGTGTCTCTGAGCTGTGCTGTGAAGGGCTTCTACCC |
| | | AAGCGACATCGCCGTCGAGTGGGAGAGCAATGGCCAGCC |
| | | AGAGAATAACTACAAAACAACACCTCCAGTGCTGGACAG |
| | | CGATGGCAGCTTCTTCCTCGTCAGCAAGCTGACAGTGGAT |
| | | AAGTCCAGATGGCAGCAAGGCAACGTCTTCAGCTGCTCCG |
| | | TGATGCACGAGGCTCTGCACAATCACTACACTCAGAAGTC |
| | | TCTGTCTCTGTCCCCCGGCAAGTGA |
| 232 | DNA encoding | ATGGACCCTAAGGGCTCTCTGAGCTGGAGGATTCTGCT |
| | IL-12p40(K104E)- | GTTTCTGTCTCTGGCTTTCGAGCTCAGCTACGGAATCT |
| | 4G-Fc (LALADS- | GGGAACTGAAGAAGGACGTCTACGTCGTGGAGCTGGATT |
| | KIH Hole: Y349C- | GGTATCCAGATGCTCCCGGCGAAATGGTGGTGCTCACATG |
| | T366S-L368A- | CGATACTCCAGAAGAGGACGGCATCACATGGACACTGGA |
| | Y407V) | TCAGAGCAGCGAAGTGCTGGGCTCCGGAAAGACACTGAC |
| | Features: | TATCCAAGTGAAGGAGTTTGGCGATGCTGGCCAGTACACA |
| | 1-72 encode | TGTCACAAGGGCGGAGAGGTGCTGAGCCACTCTCTGCTGC |
| | Leader A | TGCTGCACAAGAAGGAGGATGGCATCTGGTCCACAGACA |
| | 73-990 encodes | TTCTGAAGGACCAGAAGGAGCCTAAGAACGAGACATTTCT |
| | IL-12p40 | GAGGTGCGAAGCCAAGAATTATTCCGGAAGGTTTACATGC |
| | 991-1002 encodes | TGGTGGCTGACAACAATCAGCACAGACCTCACTTTCAGCG |
| | 4G linker | TGAAGTCCTCTAGGGGCAGCAGCGACCCTCAAGGAGTCAC |
| | 1003-1686 | ATGCGGCGCCGCTACTCTGTCCGCCGAAAGGGTGAGGGGC |
| | encodes | GACAACAAAGAGTACGAGTACAGCGTGGAGTGTCAAGAG |
| | (LALADS-KIH | GATAGCGCTTGTCCAGCCGCTGAAGAGTCTCTGCCAATCG |
| | Hole: Y349C- | AGGTCATGGTCGATGCCGTGCACAAGCTCAAGTACGAAA |
| | T366S-L368A- | ACTACACTAGCTCCTTCTTCATCAGAGATATCATCAAGCC |
| | Y407V) | AGATCCACCAAAGAACCTCCAGCTGAAGCCTCTGAAGAA |
| | | CTCTAGGCAAGTGGAGGTGAGCTGGGAGTATCCAGATACT |
| | | TGGTCCACTCCTCACAGCTACTTCTCTCTGACTTTCTGTGT |
| | | CCAAGTGCAAGGCAAGTCCAAGAGAGAGAAGAAGGATAG |
| | | GGTCTTCACAGACAAGACAAGCGCTACTGTGATCTGTAGG |
| | | AAGAACGCCTCCATCAGCGTGAGGGCCCAAGATAGGTAC |
| | | TACAGCTCCAGCTGGAGCGAGTGGGCTTCCGTGCCATGTA |
| | | GCGGAGGCGGAGGCGACAAGACACATACTTGCCCACCTT |
| | | GTCCAGCCCCAGAAGCTGCTGGCGGCCCTAGCGTGTTTCT |
| | | GTTCCCTCCTAAGCCTAAGGACACACTGATGATCTCTAGG |
| | | ACACCAGAGGTGACTTGTGTGGTGGTGAGCGTGAGCCATG |
| | | AAGATCCAGAGGTGAAGTTCAACTGGTACGTCGATGGCGT |
| | | GGAGGTGCATAACGCCAAGACAAAGCCTAGGGAGGAGCA |
| | | GTACAACAGCACATATAGGGTGGTGTCCGTGCTGACAGTG |
| | | CTGCACCAAGATTGGCTCAACGGCAAAGAGTACAAGTGT |
| | | AAGGTGAGCAACAAGGCCCTCCCAGCCCCTATCGAGAAG |
| | | ACAATCTCCAAGGCTAAGGGCCAGCCAAGGGAACCACAA |
| | | GTGTGCACACTCCCTCCTTCTAGGGACGAGCTGACTAAGA |
| | | ATCAAGTGAGCCTCAGCTGTGCCGTGAAGGGATTCTACCC |
| | | AAGCGACATTGCTGTGGAGTGGGAGTCCAACGGCCAGCC |
| | | AGAGAACAACTATAAGACAACACCACCAGTGCTGGATAG |
| | | CGATGGCAGCTTCTTTCTGGTGTCCAAACTGACAGTGGAC |
| | | AAGTCTAGGTGGCAGCAAGGCAACGTGTTCAGCTGTAGCG |
| | | TGATGCACGAGGCCCTCCACAACCACTACACACAGAAGTC |
| | | TCTGAGCCTCTCCCCCGGCAAGTGA |
| 233 | DNA encoding | ATGGACCCTAAGGGCTCTCTGAGCTGGAGGATTCTGCT |
| | IL-12p40(K104D)- | GTTTCTGTCTCTGGCTTTCGAGCTCAGCTACGGAATCT |
| | 4G-Fc (LALADS- | GGGAACTGAAGAAGGACGTCTACGTCGTGGAGCTGGATT |
| | KIH Hole: Y349C- | GGTATCCAGATGCTCCCGGCGAAATGGTGGTGCTCACATG |
| | T366S-L368A- | CGATACTCCAGAAGAGGACGGCATCACATGGACACTGGA |
| | Y407V) | TCAGAGCAGCGAAGTGCTGGGCTCCGGAAAGACACTGAC |
| | Features: | TATCCAAGTGAAGGAGTTTGGCGATGCTGGCCAGTACACA |
| | 1-72 encode | TGTCACAAGGGCGGAGAGGTGCTGAGCCACTCTCTGCTGC |
| | Leader A | TGCTGCACAAGAAGGAGGATGGCATCTGGTCCACAGACA |
| | 73-990 encodes | TTCTGAAGGACCAGAAGGAGCCTAAGAACGACACATTTCT |
| | IL-12p40 | GAGGTGCGAAGCCAAGAATTATTCCGGAAGGTTTACATGC |
| | 991-1002 encodes | TGGTGGCTGACAACAATCAGCACAGACCTCACTTTCAGCG |
| | 4G linker | TGAAGTCCTCTAGGGGCAGCAGCGACCCTCAAGGAGTCAC |
| | 1003-1686 | ATGCGGCGCCGCTACTCTGTCCGCCGAAAGGGTGAGGGGC |
| | encodes | GACAACAAAGAGTACGAGTACAGCGTGGAGTGTCAAGAG |
| | (LALADS-KIH | GATAGCGCTTGTCCAGCCGCTGAAGAGTCTCTGCCAATCG |
| | Hole: Y349C- | AGGTCATGGTCGATGCCGTGCACAAGCTCAAGTACGAAA |
| | T366S-L368A- | ACTACACTAGCTCCTTCTTCATCAGAGATATCATCAAGCC |
| | Y407V) | AGATCCACCAAAGAACCTCCAGCTGAAGCCTCTGAAGAA |
| | | CTCTAGGCAAGTGGAGGTGAGCTGGGAGTATCCAGATACT |
| | | TGGTCCACTCCTCACAGCTACTTCTCTCTGACTTTCTGTGT |
| | | CCAAGTGCAAGGCAAGTCCAAGAGAGAGAAGAAGGATAG |
| | | GGTCTTCACAGACAAGACAAGCGCTACTGTGATCTGTAGG |
| | | AAGAACGCCTCCATCAGCGTGAGGGCCCAAGATAGGTAC |
| | | TACAGCTCCAGCTGGAGCGAGTGGGCTTCCGTGCCATGTA |
| | | GCGGAGGCGGAGGCGACAAGACACATACTTGCCCACCTT |
| | | GTCCAGCCCCAGAAGCTGCTGGCGGCCCTAGCGTGTTTCT |
| | | GTTCCCTCCTAAGCCTAAGGACACACTGATGATCTCTAGG |
| | | ACACCAGAGGTGACTTGTGTGGTGGTGAGCGTGAGCCATG |
| | | AAGATCCAGAGGTGAAGTTCAACTGGTACGTCGATGGCGT |
| | | GGAGGTGCATAACGCCAAGACAAAGCCTAGGGAGGAGCA |
| | | GTACAACAGCACATATAGGGTGGTGTCCGTGCTGACAGTG |
| | | CTGCACCAAGATTGGCTCAACGGCAAAGAGTACAAGTGT |
| | | AAGGTGAGCAACAAGGCCCTCCCAGCCCCTATCGAGAAG |
| | | ACAATCTCCAAGGCTAAGGGCCAGCCAAGGGAACCACAA |
| | | GTGTGCACACTCCCTCCTTCTAGGGACGAGCTGACTAAGA |
| | | ATCAAGTGAGCCTCAGCTGTGCCGTGAAGGGATTCTACCC |
| | | AAGCGACATTGCTGTGGAGTGGGAGTCCAACGGCCAGCC |
| | | AGAGAACAACTATAAGACAACACCACCAGTGCTGGATAG |
| | | CGATGGCAGCTTCTTTCTGGTGTCCAAACTGACAGTGGAC |
| | | AAGTCTAGGTGGCAGCAAGGCAACGTGTTCAGCTGTAGCG |
| | | TGATGCACGAGGCCCTCCACAACCACTACACACAGAAGTC |
| | | TCTGAGCCTCTCCCCCGGCAAGTGA |
| 234 | DNA encoding | ATGGACCCTAAGGGCTCTCTGAGCTGGAGGATTCTGCT |
| | IL-12p35 | GTTTCTGAGCCTCGCTTTTGAGCTCAGCTATGGCACTC |
| | (Y161S)-15GS-Fc | CAGATCCCGGCATGTTCCCTTGTCTGCACCACAGCCAGAA |
| | (LALADS-YTE- | TCTGCTGAGGGCCGTGAGCAATATGCTGCAGAAAGCTAGG |
| | KIH Knob: | CAGACACTGGAGTTCTACCCTTGCACTAGCGAGGAGATCG |
| | S354C-T366W) | ATCACGAGGATATCACAAAGGACAAGACATCCACTGTGG |
| | Features: | AGGCTTGTCTGCCACTGGAGCTCACTAAGAACGAGAGCTG |
| | 1-72 encodes | TCTGAACTCTAGGGAAACAAGCTTCATCACTAATGGCAGC |
| | Leader A | TGTCTGGCTTCTAGGAAGACATCCTTCATGATGGCTCTGTG |
| | 73-645 encodes | TCTGAGCTCCATCTACGAGGATCTGAAGATGTACCAAGTG |
| | IL-12p35 | GAGTTCAAGACAATGAACGCCAAGCTGCTGATGGACCCTA |
| | 646-690 encodes | AGAGACAGATCTTTCTGGACCAGAACATGCTGGCCGTCAT |
| | 15GS linker | CGATGAACTGATGCAAGCTCTGAACTTCAACAGCGAGACA |
| | 691-1374 encodes | GTGCCTCAGAAGAGCTCTCTGGAGGAGCCAGACTTCAGCA |
| | LALADS-KIH | AGACAAAGATTAAACTGTGTATTCTGCTGCACGCCTTTAG |
| | Knob: S354C- | GATTAGGGCTGTCACTATCGATAGGGTGATGAGCTATCTG |
| | T366W | AATGCTAGCGGCGGAGGAGGCAGCGGAGGAGGAGGAA |
| | | GCGGAGGAGGCGGAAGCGACAAGACTCACACATGTCCA |
| | | CCTTGTCCAGCCCCAGAGGCTGCTGGAGGACCTTCCGTCT |
| | | TTCTGTTCCCACCTAAGCCTAAGGACACACTCTACATCAC |
| | | CAGGGAGCCAGAGGTGACATGTGTGGTCGTGTCCGTGAGC |
| | | CACGAGGACCCAGAGGTGAAGTTCAACTGGTACGTGGAC |
| | | GGCGTGGAAGTGCACAATGCCAAGACAAAGCCAAGGGAG |
| | | GAGCAATACAACAGCACTTATAGGGTGGTGAGCGTGCTG |
| | | ACAGTGCTGCATCAAGATTGGCTGAACGGCAAGGAGTAC |
| | | AAATGCAAGGTGAGCAATAAGGCTCTGCCAGCCCCTATCG |
| | | AGAAGACTATCTCCAAGGCTAAGGGCCAGCCTAGGGAGC |
| | | CACAAGTGTACACTCTGCCTCCTTGTAGGGATGAGCTGAC |
| | | TAAGAACCAAGTGTCTCTGTGGTGTCTGGTGAAAGGATTC |
| | | TACCCTAGCGACATCGCCGTCGAGTGGGAGTCCAACGGCC |
| | | AGCCAGAGAACAACTACAAGACAACACCACCAGTGCTGG |
| | | ACTCCGATGGCTCCTTCTTTCTGTACAGCAAGCTGACTGTC |
| | | GACAAGTCTAGGTGGCAGCAAGGCAACGTGTTCAGCTGTA |
| | | GCGTGATGCATGAGGCTCTGCACAATCACTACACACAGAA |
| | | GTCTCTGTCTCTGAGCCCCGGCAAATGA |
| 235 | DNA encoding | ATGGACCCTAAGGGCTCTCTGAGCTGGAGGATTCTGCT |
| | IL-12p35 | GTTTCTGAGCCTCGCTTTTGAGCTCAGCTATGGCACTC |
| | (Y161T)-15GS-Fc | CAGATCCCGGCATGTTCCCTTGTCTGCACCACAGCCAGAA |
| | (LALADS-YTE- | TCTGCTGAGGGCCGTGAGCAATATGCTGCAGAAAGCTAGG |
| | KIH Knob: | CAGACACTGGAGTTCTACCCTTGCACTAGCGAGGAGATCG |
| | S354C-T366W) | ATCACGAGGATATCACAAAGGACAAGACATCCACTGTGG |
| | Features: | AGGCTTGTCTGCCACTGGAGCTCACTAAGAACGAGAGCTG |
| | 1-72 encodes | TCTGAACTCTAGGGAAACAAGCTTCATCACTAATGGCAGC |
| | Leader A | TGTCTGGCTTCTAGGAAGACATCCTTCATGATGGCTCTGTG |
| | 73-645 encodes | TCTGAGCTCCATCTACGAGGATCTGAAGATGTACCAAGTG |
| | IL-12p35 | GAGTTCAAGACAATGAACGCCAAGCTGCTGATGGACCCTA |
| | 646-690 encodes | AGAGACAGATCTTTCTGGACCAGAACATGCTGGCCGTCAT |
| | 15GS linker | CGATGAACTGATGCAAGCTCTGAACTTCAACAGCGAGACA |
| | 691-1374 encodes | GTGCCTCAGAAGAGCTCTCTGGAGGAGCCAGACTTCACTA |
| | LALADS-KIH | AGACAAAGATTAAACTGTGTATTCTGCTGCACGCCTTTAG |
| | Knob: S354C- | GATTAGGGCTGTCACTATCGATAGGGTGATGAGCTATCTG |
| | T366W | AATGCTAGCGGCGGAGGAGGCAGCGGAGGAGGAGGAA |
| | | GCGGAGGAGGCGGAAGCGACAAGACTCACACATGTCCA |
| | | CCTTGTCCAGCCCCAGAGGCTGCTGGAGGACCTTCCGTCT |
| | | TTCTGTTCCCACCTAAGCCTAAGGACACACTCTACATCAC |
| | | CAGGGAGCCAGAGGTGACATGTGTGGTCGTGTCCGTGAGC |
| | | CACGAGGACCCAGAGGTGAAGTTCAACTGGTACGTGGAC |
| | | GGCGTGGAAGTGCACAATGCCAAGACAAAGCCAAGGGAG |
| | | GAGCAATACAACAGCACTTATAGGGTGGTGAGCGTGCTG |
| | | ACAGTGCTGCATCAAGATTGGCTGAACGGCAAGGAGTAC |
| | | AAATGCAAGGTGAGCAATAAGGCTCTGCCAGCCCCTATCG |
| | | AGAAGACTATCTCCAAGGCTAAGGGCCAGCCTAGGGAGC |
| | | CACAAGTGTACACTCTGCCTCCTTGTAGGGATGAGCTGAC |
| | | TAAGAACCAAGTGTCTCTGTGGTGTCTGGTGAAAGGATTC |
| | | TACCCTAGCGACATCGCCGTCGAGTGGGAGTCCAACGGCC |
| | | AGCCAGAGAACAACTACAAGACAACACCACCAGTGCTGG |
| | | ACTCCGATGGCTCCTTCTTTCTGTACAGCAAGCTGACTGTC |
| | | GACAAGTCTAGGTGGCAGCAAGGCAACGTGTTCAGCTGTA |
| | | GCGTGATGCATGAGGCTCTGCACAATCACTACACACAGAA |
| | | GTCTCTGTCTCTGAGCCCCGGCAAATGA |
| 236 | DNA encoding | ATGGACCCTAAGGGCAGCCTCAGCTGGAGGATTCTGC |
| | IL-12p35 | TGTTCCTCTCTCTGGCCTTTGAACTGAGCTACGGCCGC |
| | (+hexapeptide)- | AACCTGCCGGTGGCGACACCAGATCCCGGCATGTTCCCT |
| | 15GS-Fc | TGCCTCCATCACAGCCAGAATCTGCTGAGGGCCGTCAGCA |
| | (LALADS-YTE- | ATATGCTGCAGAAGGCTAGGCAGACACTCGAGTTCTATCC |
| | KIH Knob: | TTGCACAAGCGAGGAGATCGACCATGAGGATATCACTAA |
| | S354C-T366W) | GGACAAGACTAGCACTGTGGAGGCTTGTCTCCCACTGGAA |
| | Features: | CTGACTAAGAACGAGAGCTGTCTGAACTCTAGGGAGACA |
| | 1-72 encodes | AGCTTCATCACTAACGGCAGCTGTCTCGCCTCTAGGAAGA |
| | Leader A | CTTCCTTCATGATGGCTCTGTGCCTCAGCTCCATCTACGAA |
| | 73-662 encodes | GATCTGAAGATGTACCAAGTGGAGTTTAAAACTATGAACG |
| | IL-12p35 | CCAAGCTGCTGATGGACCCTAAGAGGCAGATCTTTCTCGA |
| | 664-708 encodes | CCAGAACATGCTGGCTGTCATCGACGAACTCATGCAAGCC |
| | 15GS linker | CTCAATTTCAACAGCGAGACTGTCCCTCAGAAGTCCTCTC |
| | 709-1392 encodes | TGGAGGAACCAGATTTCTACAAGACTAAGATCAAGCTCTG |
| | LALADS-KIH | TATTCTGCTGCACGCCTTTAGGATTAGGGCTGTCACAATC |
| | Knob: S354C- | GATAGGGTCATGAGCTATCTGAATGCCAGCGGCGGAGGA |
| | T366W | GGCAGCGGAGGAGGAGGAAGCGGAGGCGGCGGAAGC |
| | | GACAAGACTCACACATGCCCTCCTTGTCCAGCCCCAGAAG |
| | | CTGCCGGAGGACCTTCCGTCTTTCTGTTCCCACCTAAGCCT |
| | | AAAGACACACTCTACATCACCAGGGAGCCAGAGGTGACA |
| | | TGTGTGGTCGTGTCCGTCAGCCACGAGGACCCAGAGGTGA |
| | | AGTTTAACTGGTACGTGGACGGAGTGGAAGTGCACAATGC |
| | | CAAGACAAAGCCAAGGGAGGAGCAATACAACAGCACTTA |
| | | TAGGGTCGTGAGCGTGCTGACAGTGCTGCACCAAGATTGG |
| | | CTCAATGGCAAGGAGTATAAGTGCAAAGTGTCCAACAAA |
| | | GCTCTGCCAGCTCCTATCGAGAAGACTATCTCCAAGGCCA |
| | | AGGGCCAGCCTAGGGAGCCACAAGTGTATACACTGCCAC |
| | | CATGTAGGGACGAGCTGACAAAGAACCAAGTGTCTCTGTG |
| | | GTGTCTGGTGAAGGGCTTCTACCCTAGCGATATTGCCGTG |
| | | GAGTGGGAGTCCAACGGCCAGCCAGAGAATAACTACAAG |
| | | ACAACACCTCCAGTGCTCGATTCCGACGGCTCCTTCTTCCT |
| | | CTACTCCAAGCTGACTGTCGACAAGTCTAGGTGGCAGCAA |
| | | GGCAATGTGTTCAGCTGTAGCGTGATGCACGAGGCTCTGC |
| | | ACAATCACTATACACAGAAGTCTCTGTCCCTCAGCCCCGG |
| | | CAAATGA |
| 237 | DNA encoding | ATGGACCCTAAGGGCTCTCTGAGCTGGAGGATTCTGCT |
| | IL-12p40-4G-Fc | CTTTCTCTCTCTGGCCTTTGAACTGAGCTACGGAATCT |
| | (LALADS-YTE- | GGGAGCTGAAGAAAGACGTCTACGTGGTCGAGCTGGACT |
| | KIH Hole: Y349C- | GGTACCCAGATGCCCCCGGCGAGATGGTCGTCCTCACATG |
| | T366S-L368A- | CGACACACCAGAGGAAGACGGCATCACTTGGACTCTCGAT |
| | Y407V) | CAGTCCTCCGAAGTGCTGGGCTCCGGAAAGACACTGACAA |
| | Features: | TCCAAGTGAAGGAGTTCGGCGATGCTGGACAGTACACTTG |
| | 1-72 encode | CCACAAAGGAGGAGAGGTGCTGAGCCACTCTCTGCTGCTG |
| | Leader A | CTGCACAAGAAAGAGGACGGAATCTGGTCCACAGACATT |
| | 73-990 encodes | CTCAAGGACCAAAAGGAGCCTAAGAACAAGACATTTCTG |
| | IL-12p40 | AGGTGCGAGGCCAAGAACTACAGCGGCAGATTCACTTGCT |
| | 991-1002 encodes | GGTGGCTCACAACAATCAGCACAGACCTCACATTCTCCGT |
| | 4G linker | GAAGTCCTCCAGAGGCAGCTCCGACCCACAAGGCGTGACT |
| | 1003-1686 | TGTGGAGCTGCTACACTCAGCGCCGAAAGGGTGAGGGGA |
| | encodes | GACAATAAGGAGTACGAGTATAGCGTCGAGTGCCAAGAG |
| | (LALADS-KIH | GATAGCGCTTGTCCAGCTGCCGAAGAGTCTCTGCCAATCG |
| | Hole: Y349C- | AGGTCATGGTGGATGCCGTCCACAAGCTCAAATACGAGA |
| | T366S-L368A- | ATTACACTAGCTCCTTCTTCATTAGGGACATTATTAAGCCA |
| | Y407V) | GACCCTCCTAAGAACCTCCAGCTGAAGCCACTCAAGAACT |
| | | CTAGGCAAGTGGAGGTGAGCTGGGAGTATCCAGATACTTG |
| | | GTCCACACCTCACTCCTACTTCAGCCTCACATTCTGCGTCC |
| | | AAGTGCAAGGCAAGTCCAAGAGAGAGAAGAAGGATAGG |
| | | GTCTTCACAGATAAGACAAGCGCCACAGTGATCTGTAGGA |
| | | AGAACGCCTCCATCAGCGTGAGGGCCCAAGATAGGTACT |
| | | ATTCCTCCAGCTGGAGCGAGTGGGCTAGCGTCCCTTGCAG |
| | | CGGAGGAGGCGGAGACAAAACTCATACTTGCCCACCTTG |
| | | CCCAGCCCCAGAAGCTGCTGGCGGACCATCCGTGTTTCTG |
| | | TTCCCACCTAAGCCTAAGGACACTCTGTACATCACCAGGG |
| | | AGCCAGAGGTGACTTGTGTGGTGGTGAGCGTGAGCCATGA |
| | | AGATCCAGAGGTGAAGTTCAACTGGTACGTCGATGGCGTG |
| | | GAGGTGCATAACGCCAAGACAAAACCAAGGGAGGAACAG |
| | | TACAACAGCACATATAGGGTGGTCAGCGTCCTCACTGTGC |
| | | TCCACCAAGATTGGCTCAATGGCAAAGAATACAAGTGTAA |
| | | GGTCTCCAACAAAGCTCTGCCAGCTCCAATCGAGAAGACT |
| | | ATCTCCAAGGCCAAGGGCCAACCTAGGGAGCCACAAGTG |
| | | TGCACTCTGCCACCTTCTAGGGACGAACTGACTAAGAACC |
| | | AAGTGTCTCTGAGCTGCGCCGTGAAGGGATTTTACCCTTC |
| | | CGATATCGCCGTGGAGTGGGAGAGCAATGGCCAACCAGA |
| | | GAATAATTATAAGACAACTCCTCCAGTGCTGGACTCCGAC |
| | | GGCAGCTTCTTTCTGGTCAGCAAGCTGACTGTGGACAAGT |
| | | CTAGGTGGCAGCAAGGAAACGTGTTCAGCTGTTCCGTGAT |
| | | GCATGAAGCTCTGCACAACCACTACACACAGAAGAGCCTC |
| | | AGCCTCAGCCCCGGCAAGTGA |
| 238 | IL-12p35 N- | RNLPVA |
| | terminal | |
| | hexapeptide | |
| 239 | IL-12p35 (mature | RNLPVATPDPGMFPCLHHSQNLLRAVSNMLQKARQTLEFYP |
| | wild-type Homo | CTSEEIDHEDITKDKTSTVEACLPLELTKNESCLNSRETSFITN |
| | sapiens) | GSCLASRKTSFMMALCLSSIYEDLKMYQVEFKTMNAKLLM |
| | | DPKRQIFLDQNMLAVIDELMQALNFNSETVPQKSSLEEPDFY |
| | | KTKIKLCILLHAFRIRAVTIDRVMSYLNAS |
| 240 | DNA encoding | ATGGACCCTAAGGGCTCTCTGAGCTGGAGGATTCTGCT |
| | IL-12p35 (I41S)- | GTTTCTGAGCCTCGCCTTTGAGCTGAGCTACGGCACTC |
| | 15GS-Fc | CAGATCCCGGCATGTTCCCTTGTCTGCACCACAGCCAGAA |
| | (LALADS-YTE- | TCTGCTGAGGGCTGTGAGCAATATGCTGCAGAAGGCCAGA |
| | KIH Knob: | CAGACACTCGAGTTCTACCCTTGCACAAGCGAGGAGAGCG |
| | S354C-T366W) | ACCACGAAGACATCACAAAGGATAAGACTAGCACTGTCG |
| | Features: | AGGCTTGTCTCCCTCTGGAGCTGACAAAGAACGAGAGCTG |
| | 1-72 encodes | CCTCAACTCTAGGGAGACAAGCTTCATCACTAACGGCAGC |
| | Leader A | TGTCTGGCTTCCAGAAAGACTAGCTTCATGATGGCTCTGT |
| | 73-645 encodes | GTCTGAGCAGCATCTACGAGGATCTGAAGATGTACCAAGT |
| | IL-12p35 | GGAGTTCAAAACTATGAACGCCAAGCTGCTCATGGACCCT |
| | 646-690 encodes | AAGAGGCAGATCTTTCTGGACCAGAACATGCTGGCCGTCA |
| | 15GS linker | TCGACGAGCTGATGCAAGCCCTCAATTTCAACAGCGAGAC |
| | 691-1374 encodes | TGTCCCTCAGAAGTCCTCTCTGGAGGAGCCAGACTTCTAT |
| | LALADS-YTE- | AAGACTAAAATCAAACTCTGTATTCTGCTGCACGCCTTCA |
| | KIH Knob: | GAATCAGAGCCGTGACAATCGATAGGGTGATGAGCTATCT |
| | S354C-T366W | GAATGCTAGCGGCGGAGGAGGCAGCGGAGGAGGCGGA |
| | | AGCGGCGGAGGCGGCTCCGGACAAGACTCACACATGCC |
| | | CTCCTTGTCCAGCCCCAGAAGCTGCCGGAGGACCTTCCGT |
| | | CTTTCTGTTCCCACCTAAGCCTAAAGACACACTCTACATCA |
| | | CCAGGGAGCCAGAGGTGACATGTGTGGTCGTGTCCGTCAG |
| | | CCACGAGGACCCAGAGGTGAAGTTTAACTGGTACGTGGA |
| | | CGGAGTGGAAGTGCACAATGCCAAGACAAAGCCAAGGGA |
| | | GGAGCAATACAACAGCACTTATAGGGTCGTGAGCGTGCTG |
| | | ACAGTGCTGCACCAAGATTGGCTCAATGGCAAGGAGTATA |
| | | AGTGCAAAGTGTCCAACAAAGCTCTGCCAGCTCCTATCGA |
| | | GAAGACTATCTCCAAGGCCAAGGGCCAGCCTAGGGAGCC |
| | | ACAAGTGTATACACTGCCACCATGTAGGGACGAGCTGACA |
| | | AAGAACCAAGTGTCTCTGTGGTGTCTGGTGAAGGGCTTCT |
| | | ACCCTAGCGATATTGCCGTGGAGTGGGAGTCCAACGGCCA |
| | | GCCAGAGAATAACTACAAGACAACACCTCCAGTGCTCGAT |
| | | TCCGACGGCTCCTTCTTCCTCTACTCCAAGCTGACTGTCGA |
| | | CAAGTCTAGGTGGCAGCAAGGCAATGTGTTCAGCTGTAGC |
| | | GTGATGCACGAGGCTCTGCACAATCACTATACACAGAAGT |
| | | CTCTGTCCCTCAGCCCCGGCAAATGA |
| 241 | DNA encoding | ATGGACCCTAAGGGCTCTCTGAGCTGGAGGATTCTGCT |
| | IL-12p35 (E44R)- | GTTTCTGAGCCTCGCCTTTGAGCTGAGCTACGGCACTC |
| | 15GS-Fc | CAGATCCCGGCATGTTCCCTTGTCTGCACCACAGCCAGAA |
| | (LALADS-YTE- | TCTGCTGAGGGCTGTGAGCAATATGCTGCAGAAGGCCAGA |
| | KIH Knob: | CAGACACTCGAGTTCTACCCTTGCACAAGCGAGGAGATCG |
| | S354C-T366W) | ACCACAGaGACATCACAAAGGATAAGACTAGCACTGTCGA |
| | Features: | GGCTTGTCTCCCTCTGGAGCTGACAAAGAACGAGAGCTGC |
| | 1-72 encodes | CTCAACTCTAGGGAGACAAGCTTCATCACTAACGGCAGCT |
| | Leader A | GTCTGGCTTCCAGAAAGACTAGCTTCATGATGGCTCTGTG |
| | 73-645 encodes | TCTGAGCAGCATCTACGAGGATCTGAAGATGTACCAAGTG |
| | IL-12p35 | GAGTTCAAAACTATGAACGCCAAGCTGCTCATGGACCCTA |
| | 646-690 encodes | AGAGGCAGATCTTTCTGGACCAGAACATGCTGGCCGTCAT |
| | 15GS linker | CGACGAGCTGATGCAAGCCCTCAATTTCAACAGCGAGACT |
| | 691-1374 encodes | GTCCCTCAGAAGTCCTCTCTGGAGGAGCCAGACTTCTATA |
| | LALADS-YTE- | AGACTAAAATCAAACTCTGTATTCTGCTGCACGCCTTCAG |
| | KIH Knob: | AATCAGAGCCGTGACAATCGATAGGGTGATGAGCTATCTG |
| | S354C-T366W | AATGCTAGCGGCGGAGGAGGCAGCGGAGGAGGCGGAA |
| | | GCGGCGGAGGCGGCTCCGACAAGACTCACACATGCCCT |
| | | CCTTGTCCAGCCCCAGAAGCTGCCGGAGGACCTTCCGTCT |
| | | TTCTGTTCCCACCTAAGCCTAAAGACACACTCTACATCAC |
| | | CAGGGAGCCAGAGGTGACATGTGTGGTCGTGTCCGTCAGC |
| | | CACGAGGACCCAGAGGTGAAGTTTAACTGGTACGTGGAC |
| | | GGAGTGGAAGTGCACAATGCCAAGACAAAGCCAAGGGAG |
| | | GAGCAATACAACAGCACTTATAGGGTCGTGAGCGTGCTGA |
| | | CAGTGCTGCACCAAGATTGGCTCAATGGCAAGGAGTATAA |
| | | GTGCAAAGTGTCCAACAAAGCTCTGCCAGCTCCTATCGAG |
| | | AAGACTATCTCCAAGGCCAAGGGCCAGCCTAGGGAGCCA |
| | | CAAGTGTATACACTGCCACCATGTAGGGACGAGCTGACAA |
| | | AGAACCAAGTGTCTCTGTGGTGTCTGGTGAAGGGCTTCTA |
| | | CCCTAGCGATATTGCCGTGGAGTGGGAGTCCAACGGCCAG |
| | | CCAGAGAATAACTACAAGACAACACCTCCAGTGCTCGATT |
| | | CCGACGGCTCCTTCTTCCTCTACTCCAAGCTGACTGTCGAC |
| | | AAGTCTAGGTGGCAGCAAGGCAATGTGTTCAGCTGTAGCG |
| | | TGATGCACGAGGCTCTGCACAATCACTATACACAGAAGTC |
| | | TCTGTCCCTCAGCCCCGGCAAATGA |
| 242 | DNA encoding | ATGGACCCTAAGGGCTCTCTGAGCTGGAGGATTCTGCT |
| | IL-12p35 | GTTTCTGAGCCTCGCTTTTGAGCTCAGCTATGGCACTC |
| | (D159K)-15GS-Fc | CAGATCCCGGCATGTTCCCTTGTCTGCACCACAGCCAGAA |
| | (LALADS-YTE- | TCTGCTGAGGGCCGTGAGCAATATGCTGCAGAAAGCTAGG |
| | KIH Knob: | CAGACACTGGAGTTCTACCCTTGCACTAGCGAGGAGATCG |
| | S354C-T366W) | ATCACGAGGATATCACAAAGGACAAGACATCCACTGTGG |
| | Features: | AGGCTTGTCTGCCACTGGAGCTCACTAAGAACGAGAGCTG |
| | 1-72 encodes | TCTGAACTCTAGGGAAACAAGCTTCATCACTAATGGCAGC |
| | Leader A | TGTCTGGCTTCTAGGAAGACATCCTTCATGATGGCTCTGTG |
| | 73-645 encodes | TCTGAGCTCCATCTACGAGGATCTGAAGATGTACCAAGTG |
| | IL-12p35 | GAGTTCAAGACAATGAACGCCAAGCTGCTGATGGACCCTA |
| | 646-690 encodes | AGAGACAGATCTTTCTGGACCAGAACATGCTGGCCGTCAT |
| | 15GS linker | CGATGAACTGATGCAAGCTCTGAACTTCAACAGCGAGACA |
| | 691-1374 encodes | GTGCCTCAGAAGAGCTCTCTGGAGGAGCCAAAGTTCTACA |
| | LALADS-YTE- | AGACAAAGATTAAACTGTGTATTCTGCTGCACGCCTTTAG |
| | KIH Knob: | GATTAGGGCTGTCACTATCGATAGGGTGATGAGCTATCTG |
| | S354C-T366W | AATGCTAGCGGCGGAGGAGGCAGCGGAGGAGGAGGAA |
| | | GCGGAGGAGGCGGAAGCGACAAGACTCACACATGCCCT |
| | | CCTTGTCCAGCCCCAGAAGCTGCCGGAGGACCTTCCGTCT |
| | | TTCTGTTCCCACCTAAGCCTAAAGACACACTCTACATCAC |
| | | CAGGGAGCCAGAGGTGACATGTGTGGTCGTGTCCGTCAGC |
| | | CACGAGGACCCAGAGGTGAAGTTTAACTGGTACGTGGAC |
| | | GGAGTGGAAGTGCACAATGCCAAGACAAAGCCAAGGGAG |
| | | GAGCAATACAACAGCACTTATAGGGTCGTGAGCGTGCTGA |
| | | CAGTGCTGCACCAAGATTGGCTCAATGGCAAGGAGTATAA |
| | | GTGCAAAGTGTCCAACAAAGCTCTGCCAGCTCCTATCGAG |
| | | AAGACTATCTCCAAGGCCAAGGGCCAGCCTAGGGAGCCA |
| | | CAAGTGTATACACTGCCACCATGTAGGGACGAGCTGACAA |
| | | AGAACCAAGTGTCTCTGTGGTGTCTGGTGAAGGGCTTCTA |
| | | CCCTAGCGATATTGCCGTGGAGTGGGAGTCCAACGGCCAG |
| | | CCAGAGAATAACTACAAGACAACACCTCCAGTGCTCGATT |
| | | CCGACGGCTCCTTCTTCCTCTACTCCAAGCTGACTGTCGAC |
| | | AAGTCTAGGTGGCAGCAAGGCAATGTGTTCAGCTGTAGCG |
| | | TGATGCACGAGGCTCTGCACAATCACTATACACAGAAGTC |
| | | TCTGTCCCTCAGCCCCGGCAAATGA |
| 243 | DNA encoding | ATGGACCCTAAGGGCTCTCTGAGCTGGAGGATTCTGCT |
| | IL-12p35 | GTTTCTGAGCCTCGCTTTTGAGCTCAGCTATGGCACTC |
| | (D159R)-15GS-Fc | CAGATCCCGGCATGTTCCCTTGTCTGCACCACAGCCAGAA |
| | (LALADS-YTE- | TCTGCTGAGGGCCGTGAGCAATATGCTGCAGAAAGCTAGG |
| | KIH Knob: | CAGACACTGGAGTTCTACCCTTGCACTAGCGAGGAGATCG |
| | S354C-T366W) | ATCACGAGGATATCACAAAGGACAAGACATCCACTGTGG |
| | Features: | AGGCTTGTCTGCCACTGGAGCTCACTAAGAACGAGAGCTG |
| | 1-72 encodes | TCTGAACTCTAGGGAAACAAGCTTCATCACTAATGGCAGC |
| | Leader A | TGTCTGGCTTCTAGGAAGACATCCTTCATGATGGCTCTGTG |
| | 73-645 encodes | TCTGAGCTCCATCTACGAGGATCTGAAGATGTACCAAGTG |
| | IL-12p35 | GAGTTCAAGACAATGAACGCCAAGCTGCTGATGGACCCTA |
| | 646-690 encodes | AGAGACAGATCTTTCTGGACCAGAACATGCTGGCCGTCAT |
| | 15GS linker | CGATGAACTGATGCAAGCTCTGAACTTCAACAGCGAGACA |
| | 691-1374 encodes | GTGCCTCAGAAGAGCTCTCTGGAGGAGCCAAGGTTCTACA |
| | LALADS-YTE- | AGACAAAGATTAAACTGTGTATTCTGCTGCACGCCTTTAG |
| | KIH Knob: | GATTAGGGCTGTCACTATCGATAGGGTGATGAGCTATCTG |
| | S354C-T366W | AATGCTAGCGGCGGAGGAGGCAGCGGAGGAGGAGGAA |
| | | GCGGAGGAGGCGGAAGCGACAAGACTCACACATGCCCT |
| | | CCTTGTCCAGCCCCAGAAGCTGCCGGAGGACCTTCCGTCT |
| | | TTCTGTTCCCACCTAAGCCTAAAGACACACTCTACATCAC |
| | | CAGGGAGCCAGAGGTGACATGTGTGGTCGTGTCCGTCAGC |
| | | CACGAGGACCCAGAGGTGAAGTTTAACTGGTACGTGGAC |
| | | GGAGTGGAAGTGCACAATGCCAAGACAAAGCCAAGGGAG |
| | | GAGCAATACAACAGCACTTATAGGGTCGTGAGCGTGCTGA |
| | | CAGTGCTGCACCAAGATTGGCTCAATGGCAAGGAGTATAA |
| | | GTGCAAAGTGTCCAACAAAGCTCTGCCAGCTCCTATCGAG |
| | | AAGACTATCTCCAAGGCCAAGGGCCAGCCTAGGGAGCCA |
| | | CAAGTGTATACACTGCCACCATGTAGGGACGAGCTGACAA |
| | | AGAACCAAGTGTCTCTGTGGTGTCTGGTGAAGGGCTTCTA |
| | | CCCTAGCGATATTGCCGTGGAGTGGGAGTCCAACGGCCAG |
| | | CCAGAGAATAACTACAAGACAACACCTCCAGTGCTCGATT |
| | | CCGACGGCTCCTTCTTCCTCTACTCCAAGCTGACTGTCGAC |
| | | AAGTCTAGGTGGCAGCAAGGCAATGTGTTCAGCTGTAGCG |
| | | TGATGCACGAGGCTCTGCACAATCACTATACACAGAAGTC |
| | | TCTGTCCCTCAGCCCCGGCAAATGA |
| 244 | DNA encoding | ATGGACCCTAAGGGCTCTCTGAGCTGGAGGATTCTGCT |
| | IL-12p35 | GTTTCTGAGCCTCGCTTTTGAGCTCAGCTATGGCACTC |
| | (Y161A)-15GS-Fc | CAGATCCCGGCATGTTCCCTTGTCTGCACCACAGCCAGAA |
| | (LALADS-YTE- | TCTGCTGAGGGCCGTGAGCAATATGCTGCAGAAAGCTAGG |
| | KIH Knob: | CAGACACTGGAGTTCTACCCTTGCACTAGCGAGGAGATCG |
| | S354C-T366W) | ATCACGAGGATATCACAAAGGACAAGACATCCACTGTGG |
| | Features: | AGGCTTGTCTGCCACTGGAGCTCACTAAGAACGAGAGCTG |
| | 1-72 encodes | TCTGAACTCTAGGGAAACAAGCTTCATCACTAATGGCAGC |
| | Leader A | TGTCTGGCTTCTAGGAAGACATCCTTCATGATGGCTCTGTG |
| | 73-645 encodes | TCTGAGCTCCATCTACGAGGATCTGAAGATGTACCAAGTG |
| | IL-12p35 | GAGTTCAAGACAATGAACGCCAAGCTGCTGATGGACCCTA |
| | 646-690 encodes | AGAGACAGATCTTTCTGGACCAGAACATGCTGGCCGTCAT |
| | 15GS linker | CGATGAACTGATGCAAGCTCTGAACTTCAACAGCGAGACA |
| | 691-1374 encodes | GTGCCTCAGAAGAGCTCTCTGGAGGAGCCAGACTTCGCTA |
| | LALADS-YTE- | AGACAAAGATTAAACTGTGTATTCTGCTGCACGCCTTTAG |
| | KIH Knob: | GATTAGGGCTGTCACTATCGATAGGGTGATGAGCTATCTG |
| | S354C-T366W | AATGCTAGCGGCGGAGGAGGCAGCGGAGGAGGAGGAA |
| | | GCGGAGGAGGCGGAAGCGACAAGACTCACACATGCCCT |
| | | CCTTGTCCAGCCCCAGAAGCTGCCGGAGGACCTTCCGTCT |
| | | TTCTGTTCCCACCTAAGCCTAAAGACACACTCTACATCAC |
| | | CAGGGAGCCAGAGGTGACATGTGTGGTCGTGTCCGTCAGC |
| | | CACGAGGACCCAGAGGTGAAGTTTAACTGGTACGTGGAC |
| | | GGAGTGGAAGTGCACAATGCCAAGACAAAGCCAAGGGAG |
| | | GAGCAATACAACAGCACTTATAGGGTCGTGAGCGTGCTGA |
| | | CAGTGCTGCACCAAGATTGGCTCAATGGCAAGGAGTATAA |
| | | GTGCAAAGTGTCCAACAAAGCTCTGCCAGCTCCTATCGAG |
| | | AAGACTATCTCCAAGGCCAAGGGCCAGCCTAGGGAGCCA |
| | | CAAGTGTATACACTGCCACCATGTAGGGACGAGCTGACAA |
| | | AGAACCAAGTGTCTCTGTGGTGTCTGGTGAAGGGCTTCTA |
| | | CCCTAGCGATATTGCCGTGGAGTGGGAGTCCAACGGCCAG |
| | | CCAGAGAATAACTACAAGACAACACCTCCAGTGCTCGATT |
| | | CCGACGGCTCCTTCTTCCTCTACTCCAAGCTGACTGTCGAC |
| | | AAGTCTAGGTGGCAGCAAGGCAATGTGTTCAGCTGTAGCG |
| | | TGATGCACGAGGCTCTGCACAATCACTATACACAGAAGTC |
| | | TCTGTCCCTCAGCCCCGGCAAATGA |
| 245 | DNA encoding | ATGGACCCTAAGGGCAGCCTCAGCTGGAGGATTCTGC |
| | IL-12p40(D87L)- | TGTTTCTGTCTCTGGCCTTTGAGCTGAGCTATGGCATT |
| | 4G-Fc (LALADS- | TGGGAGCTGAAGAAGGACGTGTACGTGGTGGAGCTGGAT |
| | YTE-KIH Hole: | TGGTATCCAGATGCCCCCGGCGAGATGGTGGTGCTGACAT |
| | Y349C-T366S- | GTGACACTCCAGAGGAAGACGGCATCACTTGGACTCTCGA |
| | L368A-Y407V) | TCAGTCCTCCGAAGTGCTGGGCTCCGGAAAGACACTGACA |
| | Features: | ATCCAAGTGAAGGAGTTCGGAGATGCTGGCCAGTACACTT |
| | 1-72 encode | GCCACAAAGGCGGCGAAGTGCTGAGCCACTCTCTGCTGCT |
| | Leader A | GCTGCATAAGAAGGAGCTGGGAATCTGGTCCACAGACAT |
| | 73-990 encodes | CCTCAAGGACCAAAAGGAGCCAAAAAACAAGACATTTCT |
| | IL-12p40 | GAGGTGTGAGGCCAAGAACTACTCCGGAAGGTTTACATGC |
| | 991-1002 encodes | TGGTGGCTCACAACAATCTCCACAGACCTCACTTTCAGCG |
| | 4G linker | TGAAGAGCAGCAGAGGCAGCAGCGACCCACAAGGCGTGA |
| | 1003-1686 | CTTGTGGAGCCGCCACTCTGTCCGCTGAGAGGGTGAGGGG |
| | encodes | CGACAACAAGGAGTACGAGTACTCCGTGGAGTGCCAAGA |
| | (LALADS-YTE- | GGATAGCGCTTGTCCAGCCGCTGAGGAAAGCCTCCCTATC |
| | KIH Hole: Y349C- | GAGGTGATGGTGGACGCCGTGCACAAGCTGAAGTATGAA |
| | T366S-L368A- | AATTACACATCCAGCTTCTTCATTAGGGACATCATCAAGC |
| | Y407V) | CAGATCCTCCAAAGAACCTCCAGCTCAAGCCTCTGAAGAA |
| | | CTCTAGGCAAGTGGAGGTCAGCTGGGAGTATCCAGACACT |
| | | TGGAGCACACCTCACTCCTACTTCTCTCTGACATTCTGCGT |
| | | CCAAGTGCAAGGCAAGTCCAAGAGAGAGAAAAAGGATAG |
| | | GGTGTTCACTGACAAGACTAGCGCCACAGTGATCTGCAGA |
| | | AAGAACGCCAGCATCAGCGTGAGAGCCCAAGATAGGTAC |
| | | TATTCCTCCAGCTGGAGCGAGTGGGCTTCCGTGCCTTGTA |
| | | GCGGCGGAGGAGGCGACAAAACTCATACTTGCCCACCTT |
| | | GCCCAGCCCCAGAAGCTGCTGGCGGACCATCCGTGTTTCT |
| | | GTTCCCACCTAAGCCTAAGGACACTCTGTACATCACCAGG |
| | | GAGCCAGAGGTGACTTGTGTGGTGGTGAGCGTGAGCCATG |
| | | AAGATCCAGAGGTGAAGTTCAACTGGTACGTCGATGGCGT |
| | | GGAGGTGCATAACGCCAAGACAAAACCAAGGGAGGAACA |
| | | GTACAACAGCACATATAGGGTGGTCAGCGTCCTCACTGTG |
| | | CTCCACCAAGATTGGCTCAATGGCAAAGAATACAAGTGTA |
| | | AGGTCTCCAACAAAGCTCTGCCAGCTCCAATCGAGAAGAC |
| | | TATCTCCAAGGCCAAGGGCCAACCTAGGGAGCCACAAGT |
| | | GTGCACTCTGCCACCTTCTAGGGACGAACTGACTAAGAAC |
| | | CAAGTGTCTCTGAGCTGCGCCGTGAAGGGATTTTACCCTT |
| | | CCGATATCGCCGTGGAGTGGGAGAGCAATGGCCAACCAG |
| | | AGAATAATTATAAGACAACTCCTCCAGTGCTGGACTCCGA |
| | | CGGCAGCTTCTTTCTGGTCAGCAAGCTGACTGTGGACAAG |
| | | TCTAGGTGGCAGCAAGGAAACGTGTTCAGCTGTTCCGTGA |
| | | TGCATGAAGCTCTGCACAACCACTACACACAGAAGAGCCT |
| | | CAGCCTCAGCCCCGGCAAGTGA |
| 246 | DNA encoding | ATGGACCCTAAGGGCTCTCTGAGCTGGAGGATTCTGCT |
| | IL-12p40(K104E)- | GTTTCTGTCTCTGGCTTTCGAGCTCAGCTACGGAATCT |
| | 4G-Fc (LALADS- | GGGAACTGAAGAAGGACGTCTACGTCGTGGAGCTGGATT |
| | YTE-KIH Hole: | GGTATCCAGATGCTCCCGGCGAAATGGTGGTGCTCACATG |
| | Y349C-T366S- | CGATACTCCAGAAGAGGACGGCATCACATGGACACTGGA |
| | L368A-Y407V) | TCAGAGCAGCGAAGTGCTGGGCTCCGGAAAGACACTGAC |
| | Features: | TATCCAAGTGAAGGAGTTTGGCGATGCTGGCCAGTACACA |
| | 1-72 encode | TGTCACAAGGGCGGAGAGGTGCTGAGCCACTCTCTGCTGC |
| | Leader A | TGCTGCACAAGAAGGAGGATGGCATCTGGTCCACAGACA |
| | 73-990 encodes | TTCTGAAGGACCAGAAGGAGCCTAAGAACGAGACATTTCT |
| | IL-12p40 | GAGGTGCGAAGCCAAGAATTATTCCGGAAGGTTTACATGC |
| | 991-1002 encodes | TGGTGGCTGACAACAATCAGCACAGACCTCACTTTCAGCG |
| | 4G linker | TGAAGTCCTCTAGGGGCAGCAGCGACCCTCAAGGAGTCAC |
| | 1003-1686 | ATGCGGCGCCGCTACTCTGTCCGCCGAAAGGGTGAGGGGC |
| | encodes | GACAACAAAGAGTACGAGTACAGCGTGGAGTGTCAAGAG |
| | (LALADS-YTE- | GATAGCGCTTGTCCAGCCGCTGAAGAGTCTCTGCCAATCG |
| | KIH Hole: Y349C- | AGGTCATGGTCGATGCCGTGCACAAGCTCAAGTACGAAA |
| | T366S-L368A- | ACTACACTAGCTCCTTCTTCATCAGAGATATCATCAAGCC |
| | Y407V) | AGATCCACCAAAGAACCTCCAGCTGAAGCCTCTGAAGAA |
| | | CTCTAGGCAAGTGGAGGTGAGCTGGGAGTATCCAGATACT |
| | | TGGTCCACTCCTCACAGCTACTTCTCTCTGACTTTCTGTGT |
| | | CCAAGTGCAAGGCAAGTCCAAGAGAGAGAAGAAGGATAG |
| | | GGTCTTCACAGACAAGACAAGCGCTACTGTGATCTGTAGG |
| | | AAGAACGCCTCCATCAGCGTGAGGGCCCAAGATAGGTAC |
| | | TACAGCTCCAGCTGGAGCGAGTGGGCTTCCGTGCCATGTA |
| | | GCGGAGGCGGAGGCGACAAAACTCATACTTGCCCACCTT |
| | | GCCCAGCCCCAGAAGCTGCTGGCGGACCATCCGTGTTTCT |
| | | GTTCCCACCTAAGCCTAAGGACACTCTGTACATCACCAGG |
| | | GAGCCAGAGGTGACTTGTGTGGTGGTGAGCGTGAGCCATG |
| | | AAGATCCAGAGGTGAAGTTCAACTGGTACGTCGATGGCGT |
| | | GGAGGTGCATAACGCCAAGACAAAACCAAGGGAGGAACA |
| | | GTACAACAGCACATATAGGGTGGTCAGCGTCCTCACTGTG |
| | | CTCCACCAAGATTGGCTCAATGGCAAAGAATACAAGTGTA |
| | | AGGTCTCCAACAAAGCTCTGCCAGCTCCAATCGAGAAGAC |
| | | TATCTCCAAGGCCAAGGGCCAACCTAGGGAGCCACAAGT |
| | | GTGCACTCTGCCACCTTCTAGGGACGAACTGACTAAGAAC |
| | | CAAGTGTCTCTGAGCTGCGCCGTGAAGGGATTTTACCCTT |
| | | CCGATATCGCCGTGGAGTGGGAGAGCAATGGCCAACCAG |
| | | AGAATAATTATAAGACAACTCCTCCAGTGCTGGACTCCGA |
| | | CGGCAGCTTCTTTCTGGTCAGCAAGCTGACTGTGGACAAG |
| | | TCTAGGTGGCAGCAAGGAAACGTGTTCAGCTGTTCCGTGA |
| | | TGCATGAAGCTCTGCACAACCACTACACACAGAAGAGCCT |
| | | CAGCCTCAGCCCCGGCAAGTGA |
| 247 | DNA encoding | ATGGACCCTAAGGGCTCTCTGAGCTGGAGGATTCTGCT |
| | IL-12p40(K104D)- | GTTTCTGTCTCTGGCTTTCGAGCTCAGCTACGGAATCT |
| | 4G-Fc (LALADS- | GGGAACTGAAGAAGGACGTCTACGTCGTGGAGCTGGATT |
| | YTE-KIH Hole: | GGTATCCAGATGCTCCCGGCGAAATGGTGGTGCTCACATG |
| | Y349C-T366S- | CGATACTCCAGAAGAGGACGGCATCACATGGACACTGGA |
| | L368A-Y407V) | TCAGAGCAGCGAAGTGCTGGGCTCCGGAAAGACACTGAC |
| | Features: | TATCCAAGTGAAGGAGTTTGGCGATGCTGGCCAGTACACA |
| | 1-72 encode | TGTCACAAGGGCGGAGAGGTGCTGAGCCACTCTCTGCTGC |
| | Leader A | TGCTGCACAAGAAGGAGGATGGCATCTGGTCCACAGACA |
| | 73-990 encodes | TTCTGAAGGACCAGAAGGAGCCTAAGAACGACACATTTCT |
| | IL-12p40 | GAGGTGCGAAGCCAAGAATTATTCCGGAAGGTTTACATGC |
| | 991-1002 encodes | TGGTGGCTGACAACAATCAGCACAGACCTCACTTTCAGCG |
| | 4G linker | TGAAGTCCTCTAGGGGCAGCAGCGACCCTCAAGGAGTCAC |
| | 1003-1686 | ATGCGGCGCCGCTACTCTGTCCGCCGAAAGGGTGAGGGGC |
| | encodes | GACAACAAAGAGTACGAGTACAGCGTGGAGTGTCAAGAG |
| | (LALADS-YTE- | GATAGCGCTTGTCCAGCCGCTGAAGAGTCTCTGCCAATCG |
| | KIH Hole: Y349C- | AGGTCATGGTCGATGCCGTGCACAAGCTCAAGTACGAAA |
| | T366S-L368A- | ACTACACTAGCTCCTTCTTCATCAGAGATATCATCAAGCC |
| | Y407V) | AGATCCACCAAAGAACCTCCAGCTGAAGCCTCTGAAGAA |
| | | CTCTAGGCAAGTGGAGGTGAGCTGGGAGTATCCAGATACT |
| | | TGGTCCACTCCTCACAGCTACTTCTCTCTGACTTTCTGTGT |
| | | CCAAGTGCAAGGCAAGTCCAAGAGAGAGAAGAAGGATAG |
| | | GGTCTTCACAGACAAGACAAGCGCTACTGTGATCTGTAGG |
| | | AAGAACGCCTCCATCAGCGTGAGGGCCCAAGATAGGTAC |
| | | TACAGCTCCAGCTGGAGCGAGTGGGCTTCCGTGCCATGTA |
| | | GCGGAGGCGGAGGCGACAAAACTCATACTTGCCCACCTT |
| | | GCCCAGCCCCAGAAGCTGCTGGCGGACCATCCGTGTTTCT |
| | | GTTCCCACCTAAGCCTAAGGACACTCTGTACATCACCAGG |
| | | GAGCCAGAGGTGACTTGTGTGGTGGTGAGCGTGAGCCATG |
| | | AAGATCCAGAGGTGAAGTTCAACTGGTACGTCGATGGCGT |
| | | GGAGGTGCATAACGCCAAGACAAAACCAAGGGAGGAACA |
| | | GTACAACAGCACATATAGGGTGGTCAGCGTCCTCACTGTG |
| | | CTCCACCAAGATTGGCTCAATGGCAAAGAATACAAGTGTA |
| | | AGGTCTCCAACAAAGCTCTGCCAGCTCCAATCGAGAAGAC |
| | | TATCTCCAAGGCCAAGGGCCAACCTAGGGAGCCACAAGT |
| | | GTGCACTCTGCCACCTTCTAGGGACGAACTGACTAAGAAC |
| | | CAAGTGTCTCTGAGCTGCGCCGTGAAGGGATTTTACCCTT |
| | | CCGATATCGCCGTGGAGTGGGAGAGCAATGGCCAACCAG |
| | | AGAATAATTATAAGACAACTCCTCCAGTGCTGGACTCCGA |
| | | CGGCAGCTTCTTTCTGGTCAGCAAGCTGACTGTGGACAAG |
| | | TCTAGGTGGCAGCAAGGAAACGTGTTCAGCTGTTCCGTGA |
| | | TGCATGAAGCTCTGCACAACCACTACACACAGAAGAGCCT |
| | | CAGCCTCAGCCCCGGCAAGTGA |
| 248 | DNA encoding | ATGGACCCTAAGGGCAGCCTCAGCTGGAGGATTCTGC |
| | IL-12p35-15GS-Fc | TGTTCCTCTCTCTGGCCTTTGAACTGAGCTACGGCACA |
| | (LALADS-YTE- | CCAGATCCCGGCATGTTCCCTTGCCTCCATCACAGCCAGA |
| | KIH Knob: | ATCTGCTGAGGGCCGTCAGCAATATGCTGCAGAAGGCTAG |
| | S354C-T366W) | GCAGACACTCGAGTTCTATCCTTGCACAAGCGAGGAGATC |
| | Features: | GACCATGAGGATATCACTAAGGACAAGACTAGCACTGTG |
| | 1-72 encodes | GAGGCTTGTCTCCCACTGGAACTGACTAAGAACGAGAGCT |
| | Leader A | GTCTGAACTCTAGGGAGACAAGCTTCATCACTAACGGCAG |
| | 73-645 encodes | CTGTCTCGCCTCTAGGAAGACTTCCTTCATGATGGCTCTGT |
| | IL-12p35 | GCCTCAGCTCCATCTACGAAGATCTGAAGATGTACCAAGT |
| | 646-690 encodes | GGAGTTTAAAACTATGAACGCCAAGCTGCTGATGGACCCT |
| | 15GS linker | AAGAGGCAGATCTTTCTCGACCAGAACATGCTGGCTGTCA |
| | 691-1374 encodes | TCGACGAACTCATGCAAGCCCTCAATTTCAACAGCGAGAC |
| | LALADS-YTE- | TGTCCCTCAGAAGTCCTCTCTGGAGGAACCAGATTTCTAC |
| | KIH Knob: | AAGACTAAGATCAAGCTCTGTATTCTGCTGCACGCCTTTA |
| | S354C-T366W | GGATTAGGGCTGTCACAATCGATAGGGTCATGAGCTATCT |
| | | GAATGCCAGCGGCGGAGGAGGCAGCGGAGGAGGAGGA |
| | | AGCGGAGGCGGCGGAAGCGACAAGACTCACACATGCCC |
| | | TCCTTGTCCAGCCCCAGAAGCTGCCGGAGGACCTTCCGTC |
| | | TTTCTGTTCCCACCTAAGCCTAAAGACACACTCTACATCAC |
| | | CAGGGAGCCAGAGGTGACATGTGTGGTCGTGTCCGTCAGC |
| | | CACGAGGACCCAGAGGTGAAGTTTAACTGGTACGTGGAC |
| | | GGAGTGGAAGTGCACAATGCCAAGACAAAGCCAAGGGAG |
| | | GAGCAATACAACAGCACTTATAGGGTCGTGAGCGTGCTGA |
| | | CAGTGCTGCACCAAGATTGGCTCAATGGCAAGGAGTATAA |
| | | GTGCAAAGTGTCCAACAAAGCTCTGCCAGCTCCTATCGAG |
| | | AAGACTATCTCCAAGGCCAAGGGCCAGCCTAGGGAGCCA |
| | | CAAGTGTATACACTGCCACCATGTAGGGACGAGCTGACAA |
| | | AGAACCAAGTGTCTCTGTGGTGTCTGGTGAAGGGCTTCTA |
| | | CCCTAGCGATATTGCCGTGGAGTGGGAGTCCAACGGCCAG |
| | | CCAGAGAATAACTACAAGACAACACCTCCAGTGCTCGATT |
| | | CCGACGGCTCCTTCTTCCTCTACTCCAAGCTGACTGTCGAC |
| | | AAGTCTAGGTGGCAGCAAGGCAATGTGTTCAGCTGTAGCG |
| | | TGATGCACGAGGCTCTGCACAATCACTATACACAGAAGTC |
| | | TCTGTCCCTCAGCCCCGGCAAATGA |
| 249 | DNA encoding | ATGGACCCTAAGGGCAGCCTCAGCTGGAGGATTCTGC |
| | IL-12p35-15GS-Fc | TGTTCCTCTCTCTGGCCTTTGAACTGAGCTACGGCACA |
| | (LALADS-KIH | CCAGATCCCGGCATGTTCCCTTGCCTCCATCACAGCCAGA |
| | Knob: S354C- | ATCTGCTGAGGGCCGTCAGCAATATGCTGCAGAAGGCTAG |
| | T366W) | GCAGACACTCGAGTTCTATCCTTGCACAAGCGAGGAGATC |
| | Features: | GACCATGAGGATATCACTAAGGACAAGACTAGCACTGTG |
| | 1-72 encodes | GAGGCTTGTCTCCCACTGGAACTGACTAAGAACGAGAGCT |
| | Leader A | GTCTGAACTCTAGGGAGACAAGCTTCATCACTAACGGCAG |
| | 73-645 encodes | CTGTCTCGCCTCTAGGAAGACTTCCTTCATGATGGCTCTGT |
| | IL-12p35 | GCCTCAGCTCCATCTACGAAGATCTGAAGATGTACCAAGT |
| | 646-690 encodes | GGAGTTTAAAACTATGAACGCCAAGCTGCTGATGGACCCT |
| | 15GS linker | AAGAGGCAGATCTTTCTCGACCAGAACATGCTGGCTGTCA |
| | 691-1374 encodes | TCGACGAACTCATGCAAGCCCTCAATTTCAACAGCGAGAC |
| | LALADS-KIH | TGTCCCTCAGAAGTCCTCTCTGGAGGAACCAGATTTCTAC |
| | Knob: S354C- | AAGACTAAGATCAAGCTCTGTATTCTGCTGCACGCCTTTA |
| | T366W | GGATTAGGGCTGTCACAATCGATAGGGTCATGAGCTATCT |
| | | GAATGCCAGCGGCGGAGGAGGCAGCGGAGGAGGAGGA |
| | | AGCGGAGGCGGCGGAAGCGACAAGACTCACACATGTCC |
| | | ACCTTGTCCAGCCCCAGAGGCTGCTGGAGGACCTTCCGTC |
| | | TTTCTGTTCCCACCTAAGCCTAAGGACACACTCATGATCTC |
| | | TAGGACACCAGAGGTGACATGTGTGGTCGTGTCCGTCAGC |
| | | CACGAGGACCCAGAGGTGAAGTTTAACTGGTACGTGGAC |
| | | GGCGTGGAAGTGCACAATGCCAAGACAAAGCCAAGGGAG |
| | | GAGCAATACAACAGCACTTATAGGGTCGTGAGCGTGCTGA |
| | | CAGTGCTGCATCAAGATTGGCTGAACGGCAAGGAGTACA |
| | | AGTGCAAGGTGAGCAACAAAGCTCTGCCAGCCCCTATCGA |
| | | GAAGACTATCAGCAAGGCCAAGGGCCAGCCTAGGGAGCC |
| | | ACAAGTGTACACACTGCCTCCTTGTAGGGACGAGCTGACT |
| | | AAGAACCAAGTGTCTCTGTGGTGTCTGGTCAAGGGCTTTT |
| | | ACCCTTCCGATATCGCCGTGGAGTGGGAATCCAACGGCCA |
| | | GCCAGAGAATAACTACAAGACAACTCCTCCAGTGCTGGAC |
| | | TCCGATGGCTCCTTCTTCCTCTACAGCAAGCTGACTGTGGA |
| | | TAAGAGCAGATGGCAGCAAGGCAACGTGTTCAGCTGTAG |
| | | CGTGATGCATGAGGCTCTGCACAACCACTACACTCAGAAG |
| | | TCTCTGTCTCTGTCCCCCGGCAAATGA |
| 250 | DNA encoding | ATGGACCCTAAGGGCAGCCTCAGCTGGAGGATTCTGC |
| | IL-12p35 | TGTTCCTCTCTCTGGCCTTTGAACTGAGCTACGGCCGC |
| | (+hexapeptide)- | AACCTGCCGGTGGCGACACCAGATCCCGGCATGTTCCCT |
| | 15GS-Fc | TGCCTCCATCACAGCCAGAATCTGCTGAGGGCCGTCAGCA |
| | (LALADS-KIH | ATATGCTGCAGAAGGCTAGGCAGACACTCGAGTTCTATCC |
| | Knob: S354C- | TTGCACAAGCGAGGAGATCGACCATGAGGATATCACTAA |
| | T366W) | GGACAAGACTAGCACTGTGGAGGCTTGTCTCCCACTGGAA |
| | Features: | CTGACTAAGAACGAGAGCTGTCTGAACTCTAGGGAGACA |
| | 1-72 encodes | AGCTTCATCACTAACGGCAGCTGTCTCGCCTCTAGGAAGA |
| | Leader A | CTTCCTTCATGATGGCTCTGTGCCTCAGCTCCATCTACGAA |
| | 73-662 encodes | GATCTGAAGATGTACCAAGTGGAGTTTAAAACTATGAACG |
| | IL-12p35 | CCAAGCTGCTGATGGACCCTAAGAGGCAGATCTTTCTCGA |
| | 664-708 encodes | CCAGAACATGCTGGCTGTCATCGACGAACTCATGCAAGCC |
| | 15GS linker | CTCAATTTCAACAGCGAGACTGTCCCTCAGAAGTCCTCTC |
| | 709-1392 encodes | TGGAGGAACCAGATTTCTACAAGACTAAGATCAAGCTCTG |
| | LALADS-KIH | TATTCTGCTGCACGCCTTTAGGATTAGGGCTGTCACAATC |
| | Knob: S354C- | GATAGGGTCATGAGCTATCTGAATGCCAGCGGCGGAGGA |
| | T366W | GGCAGCGGAGGAGGAGGAAGCGGAGGCGGCGGAAGC |
| | | GACAAGACTCACACATGTCCACCTTGTCCAGCCCCAGAGG |
| | | CTGCTGGAGGACCTTCCGTCTTTCTGTTCCCACCTAAGCCT |
| | | AAGGACACACTCATGATCTCTAGGACACCAGAGGTGACAT |
| | | GTGTGGTCGTGTCCGTCAGCCACGAGGACCCAGAGGTGAA |
| | | GTTTAACTGGTACGTGGACGGCGTGGAAGTGCACAATGCC |
| | | AAGACAAAGCCAAGGGAGGAGCAATACAACAGCACTTAT |
| | | AGGGTCGTGAGCGTGCTGACAGTGCTGCATCAAGATTGGC |
| | | TGAACGGCAAGGAGTACAAGTGCAAGGTGAGCAACAAAG |
| | | CTCTGCCAGCCCCTATCGAGAAGACTATCAGCAAGGCCAA |
| | | GGGCCAGCCTAGGGAGCCACAAGTGTACACACTGCCTCCT |
| | | TGTAGGGACGAGCTGACTAAGAACCAAGTGTCTCTGTGGT |
| | | GTCTGGTCAAGGGCTTTTACCCTTCCGATATCGCCGTGGA |
| | | GTGGGAATCCAACGGCCAGCCAGAGAATAACTACAAGAC |
| | | AACTCCTCCAGTGCTGGACTCCGATGGCTCCTTCTTCCTCT |
| | | ACAGCAAGCTGACTGTGGATAAGAGCAGATGGCAGCAAG |
| | | GCAACGTGTTCAGCTGTAGCGTGATGCATGAGGCTCTGCA |
| | | CAACCACTACACTCAGAAGTCTCTGTCTCTGTCCCCCGGC |
| | | AAATGA |
| 251 | DNA encoding | ACTCCAGATCCCGGCATGTTCCCTTGTCTGCACCACAGCC |
| | IL-12p35 (I41S) | AGAATCTGCTGAGGGCTGTGAGCAATATGCTGCAGAAGG |
| | | CCAGACAGACACTCGAGTTCTACCCTTGCACAAGCGAGGA |
| | | GAGCGACCACGAAGACATCACAAAGGATAAGACTAGCAC |
| | | TGTCGAGGCTTGTCTCCCTCTGGAGCTGACAAAGAACGAG |
| | | AGCTGCCTCAACTCTAGGGAGACAAGCTTCATCACTAACG |
| | | GCAGCTGTCTGGCTTCCAGAAAGACTAGCTTCATGATGGC |
| | | TCTGTGTCTGAGCAGCATCTACGAGGATCTGAAGATGTAC |
| | | CAAGTGGAGTTCAAAACTATGAACGCCAAGCTGCTCATGG |
| | | ACCCTAAGAGGCAGATCTTTCTGGACCAGAACATGCTGGC |
| | | CGTCATCGACGAGCTGATGCAAGCCCTCAATTTCAACAGC |
| | | GAGACTGTCCCTCAGAAGTCCTCTCTGGAGGAGCCAGACT |
| | | TCTATAAGACTAAAATCAAACTCTGTATTCTGCTGCACGC |
| | | CTTCAGAATCAGAGCCGTGACAATCGATAGGGTGATGAGC |
| | | TATCTGAATGCTAGC |
| 252 | DNA encoding | ACTCCAGATCCCGGCATGTTCCCTTGTCTGCACCACAGCC |
| | IL-12p35 (E44R) | AGAATCTGCTGAGGGCTGTGAGCAATATGCTGCAGAAGG |
| | | CCAGACAGACACTCGAGTTCTACCCTTGCACAAGCGAGGA |
| | | GATCGACCACAGaGACATCACAAAGGATAAGACTAGCACT |
| | | GTCGAGGCTTGTCTCCCTCTGGAGCTGACAAAGAACGAGA |
| | | GCTGCCTCAACTCTAGGGAGACAAGCTTCATCACTAACGG |
| | | CAGCTGTCTGGCTTCCAGAAAGACTAGCTTCATGATGGCT |
| | | CTGTGTCTGAGCAGCATCTACGAGGATCTGAAGATGTACC |
| | | AAGTGGAGTTCAAAACTATGAACGCCAAGCTGCTCATGGA |
| | | CCCTAAGAGGCAGATCTTTCTGGACCAGAACATGCTGGCC |
| | | GTCATCGACGAGCTGATGCAAGCCCTCAATTTCAACAGCG |
| | | AGACTGTCCCTCAGAAGTCCTCTCTGGAGGAGCCAGACTT |
| | | CTATAAGACTAAAATCAAACTCTGTATTCTGCTGCACGCC |
| | | TTCAGAATCAGAGCCGTGACAATCGATAGGGTGATGAGCT |
| | | ATCTGAATGCTAGC |
| | | ACTCCAGATCCCGGCATGTTCCCTTGTCTGCACCACAGCC |
| | | AGAATCTGCTGAGGGCCGTGAGCAATATGCTGCAGAAAG |
| 253 | DNA encoding | CTAGGCAGACACTGGAGTTCTACCCTTGCACTAGCGAGGA |
| | IL-12p35 (D159K) | GATCGATCACGAGGATATCACAAAGGACAAGACATCCAC |
| | | TGTGGAGGCTTGTCTGCCACTGGAGCTCACTAAGAACGAG |
| | | AGCTGTCTGAACTCTAGGGAAACAAGCTTCATCACTAATG |
| | | GCAGCTGTCTGGCTTCTAGGAAGACATCCTTCATGATGGC |
| | | TCTGTGTCTGAGCTCCATCTACGAGGATCTGAAGATGTAC |
| | | CAAGTGGAGTTCAAGACAATGAACGCCAAGCTGCTGATG |
| | | GACCCTAAGAGACAGATCTTTCTGGACCAGAACATGCTGG |
| | | CCGTCATCGATGAACTGATGCAAGCTCTGAACTTCAACAG |
| | | CGAGACAGTGCCTCAGAAGAGCTCTCTGGAGGAGCCAAA |
| | | GTTCTACAAGACAAAGATTAAACTGTGTATTCTGCTGCAC |
| | | GCCTTTAGGATTAGGGCTGTCACTATCGATAGGGTGATGA |
| | | GCTATCTGAATGCTAGC |
| 254 | DNA encoding | ACTCCAGATCCCGGCATGTTCCCTTGTCTGCACCACAGCC |
| | IL-12p35 (D159R) | AGAATCTGCTGAGGGCCGTGAGCAATATGCTGCAGAAAG |
| | | CTAGGCAGACACTGGAGTTCTACCCTTGCACTAGCGAGGA |
| | | GATCGATCACGAGGATATCACAAAGGACAAGACATCCAC |
| | | TGTGGAGGCTTGTCTGCCACTGGAGCTCACTAAGAACGAG |
| | | AGCTGTCTGAACTCTAGGGAAACAAGCTTCATCACTAATG |
| | | GCAGCTGTCTGGCTTCTAGGAAGACATCCTTCATGATGGC |
| | | TCTGTGTCTGAGCTCCATCTACGAGGATCTGAAGATGTAC |
| | | CAAGTGGAGTTCAAGACAATGAACGCCAAGCTGCTGATG |
| | | GACCCTAAGAGACAGATCTTTCTGGACCAGAACATGCTGG |
| | | CCGTCATCGATGAACTGATGCAAGCTCTGAACTTCAACAG |
| | | CGAGACAGTGCCTCAGAAGAGCTCTCTGGAGGAGCCAAG |
| | | GTTCTACAAGACAAAGATTAAACTGTGTATTCTGCTGCAC |
| | | GCCTTTAGGATTAGGGCTGTCACTATCGATAGGGTGATGA |
| | | GCTATCTGAATGCTAGC |
| 255 | DNA encoding | ACTCCAGATCCCGGCATGTTCCCTTGTCTGCACCACAGCC |
| | IL-12p35 (Y161S) | AGAATCTGCTGAGGGCCGTGAGCAATATGCTGCAGAAAG |
| | | CTAGGCAGACACTGGAGTTCTACCCTTGCACTAGCGAGGA |
| | | GATCGATCACGAGGATATCACAAAGGACAAGACATCCAC |
| | | TGTGGAGGCTTGTCTGCCACTGGAGCTCACTAAGAACGAG |
| | | AGCTGTCTGAACTCTAGGGAAACAAGCTTCATCACTAATG |
| | | GCAGCTGTCTGGCTTCTAGGAAGACATCCTTCATGATGGC |
| | | TCTGTGTCTGAGCTCCATCTACGAGGATCTGAAGATGTAC |
| | | CAAGTGGAGTTCAAGACAATGAACGCCAAGCTGCTGATG |
| | | GACCCTAAGAGACAGATCTTTCTGGACCAGAACATGCTGG |
| | | CCGTCATCGATGAACTGATGCAAGCTCTGAACTTCAACAG |
| | | CGAGACAGTGCCTCAGAAGAGCTCTCTGGAGGAGCCAGA |
| | | CTTCAGCAAGACAAAGATTAAACTGTGTATTCTGCTGCAC |
| | | GCCTTTAGGATTAGGGCTGTCACTATCGATAGGGTGATGA |
| | | GCTATCTGAATGCTAGC |
| 256 | DNA encoding | ACTCCAGATCCCGGCATGTTCCCTTGTCTGCACCACAGCC |
| | IL-12p35 (Y161T) | AGAATCTGCTGAGGGCCGTGAGCAATATGCTGCAGAAAG |
| | | CTAGGCAGACACTGGAGTTCTACCCTTGCACTAGCGAGGA |
| | | GATCGATCACGAGGATATCACAAAGGACAAGACATCCAC |
| | | TGTGGAGGCTTGTCTGCCACTGGAGCTCACTAAGAACGAG |
| | | AGCTGTCTGAACTCTAGGGAAACAAGCTTCATCACTAATG |
| | | GCAGCTGTCTGGCTTCTAGGAAGACATCCTTCATGATGGC |
| | | TCTGTGTCTGAGCTCCATCTACGAGGATCTGAAGATGTAC |
| | | CAAGTGGAGTTCAAGACAATGAACGCCAAGCTGCTGATG |
| | | GACCCTAAGAGACAGATCTTTCTGGACCAGAACATGCTGG |
| | | CCGTCATCGATGAACTGATGCAAGCTCTGAACTTCAACAG |
| | | CGAGACAGTGCCTCAGAAGAGCTCTCTGGAGGAGCCAGA |
| | | CTTCACTAAGACAAAGATTAAACTGTGTATTCTGCTGCAC |
| | | GCCTTTAGGATTAGGGCTGTCACTATCGATAGGGTGATGA |
| | | GCTATCTGAATGCTAGC |
| 257 | DNA encoding | ACTCCAGATCCCGGCATGTTCCCTTGTCTGCACCACAGCC |
| | IL-12p35 (Y161A) | AGAATCTGCTGAGGGCCGTGAGCAATATGCTGCAGAAAG |
| | | CTAGGCAGACACTGGAGTTCTACCCTTGCACTAGCGAGGA |
| | | GATCGATCACGAGGATATCACAAAGGACAAGACATCCAC |
| | | TGTGGAGGCTTGTCTGCCACTGGAGCTCACTAAGAACGAG |
| | | AGCTGTCTGAACTCTAGGGAAACAAGCTTCATCACTAATG |
| | | GCAGCTGTCTGGCTTCTAGGAAGACATCCTTCATGATGGC |
| | | TCTGTGTCTGAGCTCCATCTACGAGGATCTGAAGATGTAC |
| | | CAAGTGGAGTTCAAGACAATGAACGCCAAGCTGCTGATG |
| | | GACCCTAAGAGACAGATCTTTCTGGACCAGAACATGCTGG |
| | | CCGTCATCGATGAACTGATGCAAGCTCTGAACTTCAACAG |
| | | CGAGACAGTGCCTCAGAAGAGCTCTCTGGAGGAGCCAGA |
| | | CTTCGCTAAGACAAAGATTAAACTGTGTATTCTGCTGCAC |
| | | GCCTTTAGGATTAGGGCTGTCACTATCGATAGGGTGATGA |
| | | GCTATCTGAATGCTAGC |
| 258 | DNA encoding | ATTTGGGAGCTGAAGAAGGACGTGTACGTGGTGGAGCTG |
| | IL-12p40 (D87L) | GATTGGTATCCAGATGCCCCCGGCGAGATGGTGGTGCTGA |
| | | CATGTGACACTCCAGAGGAAGACGGCATCACTTGGACTCT |
| | | CGATCAGTCCTCCGAAGTGCTGGGCTCCGGAAAGACACTG |
| | | ACAATCCAAGTGAAGGAGTTCGGAGATGCTGGCCAGTAC |
| | | ACTTGCCACAAAGGCGGCGAAGTGCTGAGCCACTCTCTGC |
| | | TGCTGCTGCATAAGAAGGAGCTGGGAATCTGGTCCACAGA |
| | | CATCCTCAAGGACCAAAAGGAGCCAAAAAACAAGACATT |
| | | TCTGAGGTGTGAGGCCAAGAACTACTCCGGAAGGTTTACA |
| | | TGCTGGTGGCTCACAACAATCTCCACAGACCTCACTTTCA |
| | | GCGTGAAGAGCAGCAGAGGCAGCAGCGACCCACAAGGCG |
| | | TGACTTGTGGAGCCGCCACTCTGTCCGCTGAGAGGGTGAG |
| | | GGGCGACAACAAGGAGTACGAGTACTCCGTGGAGTGCCA |
| | | AGAGGATAGCGCTTGTCCAGCCGCTGAGGAAAGCCTCCCT |
| | | ATCGAGGTGATGGTGGACGCCGTGCACAAGCTGAAGTAT |
| | | GAAAATTACACATCCAGCTTCTTCATTAGGGACATCATCA |
| | | AGCCAGATCCTCCAAAGAACCTCCAGCTCAAGCCTCTGAA |
| | | GAACTCTAGGCAAGTGGAGGTCAGCTGGGAGTATCCAGA |
| | | CACTTGGAGCACACCTCACTCCTACTTCTCTCTGACATTCT |
| | | GCGTCCAAGTGCAAGGCAAGTCCAAGAGAGAGAAAAAGG |
| | | ATAGGGTGTTCACTGACAAGACTAGCGCCACAGTGATCTG |
| | | CAGAAAGAACGCCAGCATCAGCGTGAGAGCCCAAGATAG |
| | | GTACTATTCCTCCAGCTGGAGCGAGTGGGCTTCCGTGCCT |
| | | TGTAGC |
| 259 | DNA encoding | ATCTGGGAACTGAAGAAGGACGTCTACGTCGTGGAGCTG |
| | IL-12p40 (K104E) | GATTGGTATCCAGATGCTCCCGGCGAAATGGTGGTGCTCA |
| | | CATGCGATACTCCAGAAGAGGACGGCATCACATGGACAC |
| | | TGGATCAGAGCAGCGAAGTGCTGGGCTCCGGAAAGACAC |
| | | TGACTATCCAAGTGAAGGAGTTTGGCGATGCTGGCCAGTA |
| | | CACATGTCACAAGGGCGGAGAGGTGCTGAGCCACTCTCTG |
| | | CTGCTGCTGCACAAGAAGGAGGATGGCATCTGGTCCACAG |
| | | ACATTCTGAAGGACCAGAAGGAGCCTAAGAACGAGACAT |
| | | TTCTGAGGTGCGAAGCCAAGAATTATTCCGGAAGGTTTAC |
| | | ATGCTGGTGGCTGACAACAATCAGCACAGACCTCACTTTC |
| | | AGCGTGAAGTCCTCTAGGGGCAGCAGCGACCCTCAAGGA |
| | | GTCACATGCGGCGCCGCTACTCTGTCCGCCGAAAGGGTGA |
| | | GGGGCGACAACAAAGAGTACGAGTACAGCGTGGAGTGTC |
| | | AAGAGGATAGCGCTTGTCCAGCCGCTGAAGAGTCTCTGCC |
| | | AATCGAGGTCATGGTCGATGCCGTGCACAAGCTCAAGTAC |
| | | GAAAACTACACTAGCTCCTTCTTCATCAGAGATATCATCA |
| | | AGCCAGATCCACCAAAGAACCTCCAGCTGAAGCCTCTGAA |
| | | GAACTCTAGGCAAGTGGAGGTGAGCTGGGAGTATCCAGA |
| | | TACTTGGTCCACTCCTCACAGCTACTTCTCTCTGACTTTCT |
| | | GTGTCCAAGTGCAAGGCAAGTCCAAGAGAGAGAAGAAGG |
| | | ATAGGGTCTTCACAGACAAGACAAGCGCTACTGTGATCTG |
| | | TAGGAAGAACGCCTCCATCAGCGTGAGGGCCCAAGATAG |
| | | GTACTACAGCTCCAGCTGGAGCGAGTGGGCTTCCGTGCCA |
| | | TGTAGC |
| 260 | DNA encoding | ATCTGGGAACTGAAGAAGGACGTCTACGTCGTGGAGCTG |
| | IL-12p40(K104D) | GATTGGTATCCAGATGCTCCCGGCGAAATGGTGGTGCTCA |
| | | CATGCGATACTCCAGAAGAGGACGGCATCACATGGACAC |
| | | TGGATCAGAGCAGCGAAGTGCTGGGCTCCGGAAAGACAC |
| | | TGACTATCCAAGTGAAGGAGTTTGGCGATGCTGGCCAGTA |
| | | CACATGTCACAAGGGCGGAGAGGTGCTGAGCCACTCTCTG |
| | | CTGCTGCTGCACAAGAAGGAGGATGGCATCTGGTCCACAG |
| | | ACATTCTGAAGGACCAGAAGGAGCCTAAGAACGACACAT |
| | | TTCTGAGGTGCGAAGCCAAGAATTATTCCGGAAGGTTTAC |
| | | ATGCTGGTGGCTGACAACAATCAGCACAGACCTCACTTTC |
| | | AGCGTGAAGTCCTCTAGGGGCAGCAGCGACCCTCAAGGA |
| | | GTCACATGCGGCGCCGCTACTCTGTCCGCCGAAAGGGTGA |
| | | GGGGCGACAACAAAGAGTACGAGTACAGCGTGGAGTGTC |
| | | AAGAGGATAGCGCTTGTCCAGCCGCTGAAGAGTCTCTGCC |
| | | AATCGAGGTCATGGTCGATGCCGTGCACAAGCTCAAGTAC |
| | | GAAAACTACACTAGCTCCTTCTTCATCAGAGATATCATCA |
| | | AGCCAGATCCACCAAAGAACCTCCAGCTGAAGCCTCTGAA |
| | | GAACTCTAGGCAAGTGGAGGTGAGCTGGGAGTATCCAGA |
| | | TACTTGGTCCACTCCTCACAGCTACTTCTCTCTGACTTTCT |
| | | GTGTCCAAGTGCAAGGCAAGTCCAAGAGAGAGAAGAAGG |
| | | ATAGGGTCTTCACAGACAAGACAAGCGCTACTGTGATCTG |
| | | TAGGAAGAACGCCTCCATCAGCGTGAGGGCCCAAGATAG |
| | | GTACTACAGCTCCAGCTGGAGCGAGTGGGCTTCCGTGCCA |
| | | TGTAGC |
| 261 | p35 (E40K) | RNLPVATPDPGMFPCLHHSQNLLRAVSNMLQKARQTLEFYP |
| | (+hexapeptide) | CTSEKIDHEDITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNFNSETVPQKSSLEEPDFY |
| | | KTKIKLCILLHAFRIRAVTIDRVMSYLNAS |
| 262 | p35 (E40R) | RNLPVATPDPGMFPCLHHSQNLLRAVSNMLQKARQTLEFYP |
| | (+hexapeptide) | CTSERIDHEDITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNFNSETVPQKSSLEEPDFY |
| | | KTKIKLCILLHAFRIRAVTIDRVMSYLNAS |
| 263 | p35 (E40W) | RNLPVATPDPGMFPCLHHSQNLLRAVSNMLQKARQTLEFYP |
| | (+hexapeptide) | CTSEWIDHEDITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNFNSETVPQKSSLEEPDFY |
| | | KTKIKLCILLHAFRIRAVTIDRVMSYLNAS |
| 264 | p35 (I41A) | RNLPVATPDPGMFPCLHHSQNLLRAVSNMLQKARQTLEFYP |
| | (+hexapeptide) | CTSEEADHEDITKDKTSTVEACLPLELTKNESCLNSRETSFIT |
| | | NGSCLASRKTSFMMALCLSSIYEDLKMYQVEFKTMNAKLL |
| | | MDPKRQIFLDQNMLAVIDELMQALNFNSETVPQKSSLEEPDF |
| | | YKTKIKLCILLHAFRIRAVTIDRVMSYLNAS |
| 265 | p35 (I41V) | RNLPVATPDPGMFPCLHHSQNLLRAVSNMLQKARQTLEFYP |
| | (+hexapeptide) | CTSEEVDHEDITKDKTSTVEACLPLELTKNESCLNSRETSFIT |
| | | NGSCLASRKTSFMMALCLSSIYEDLKMYQVEFKTMNAKLL |
| | | MDPKRQIFLDQNMLAVIDELMQALNFNSETVPQKSSLEEPDF |
| | | YKTKIKLCILLHAFRIRAVTIDRVMSYLNAS |
| 266 | p35 (I41S) | RNLPVATPDPGMFPCLHHSQNLLRAVSNMLQKARQTLEFYP |
| | (+hexapeptide) | CTSEESDHEDITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNFNSETVPQKSSLEEPDFY |
| | | KTKIKLCILLHAFRIRAVTIDRVMSYLNAS |
| 267 | p35 (I41T) | RNLPVATPDPGMFPCLHHSQNLLRAVSNMLQKARQTLEFYP |
| | (+hexapeptide) | CTSEETDHEDITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNFNSETVPQKSSLEEPDFY |
| | | KTKIKLCILLHAFRIRAVTIDRVMSYLNAS |
| 268 | p35 (D42A) | RNLPVATPDPGMFPCLHHSQNLLRAVSNMLQKARQTLEFYP |
| | (+hexapeptide) | CTSEEIAHEDITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNFNSETVPQKSSLEEPDFY |
| | | KTKIKLCILLHAFRIRAVTIDRVMSYLNAS |
| 269 | p35 (D42L) | RNLPVATPDPGMFPCLHHSQNLLRAVSNMLQKARQTLEFYP |
| | (+hexapeptide) | CTSEEILHEDITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNFNSETVPQKSSLEEPDFY |
| | | KTKIKLCILLHAFRIRAVTIDRVMSYLNAS |
| 270 | p35 (D42W) | RNLPVATPDPGMFPCLHHSQNLLRAVSNMLQKARQTLEFYP |
| | (+hexapeptide) | CTSEEIWHEDITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNFNSETVPQKSSLEEPDFY |
| | | KTKIKLCILLHAFRIRAVTIDRVMSYLNAS |
| 271 | p35 (E44A) | RNLPVATPDPGMFPCLHHSQNLLRAVSNMLQKARQTLEFYP |
| | (+hexapeptide) | CTSEEIDHADITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNFNSETVPQKSSLEEPDFY |
| | | KTKIKLCILLHAFRIRAVTIDRVMSYLNAS |
| 272 | p35 (E44K) | RNLPVATPDPGMFPCLHHSQNLLRAVSNMLQKARQTLEFYP |
| | (+hexapeptide) | CTSEEIDHKDITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNFNSETVPQKSSLEEPDFY |
| | | KTKIKLCILLHAFRIRAVTIDRVMSYLNAS |
| 273 | p35 (E44R) | RNLPVATPDPGMFPCLHHSQNLLRAVSNMLQKARQTLEFYP |
| | (+hexapeptide) | CTSEEIDHRDITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNFNSETVPQKSSLEEPDFY |
| | | KTKIKLCILLHAFRIRAVTIDRVMSYLNAS |
| 274 | p35 (D159K) | RNLPVATPDPGMFPCLHHSQNLLRAVSNMLQKARQTLEFYP |
| | (+hexapeptide) | CTSEEIDHEDITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNFNSETVPQKSSLEEPKFY |
| | | KTKIKLCILLHAFRIRAVTIDRVMSYLNAS |
| 275 | p35 (D159R) | RNLPVATPDPGMFPCLHHSQNLLRAVSNMLQKARQTLEFYP |
| | (+hexapeptide) | CTSEEIDHEDITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNFNSETVPQKSSLEEPRFY |
| | | KTKIKLCILLHAFRIRAVTIDRVMSYLNAS |
| 276 | p35 (D159Y) | RNLPVATPDPGMFPCLHHSQNLLRAVSNMLQKARQTLEFYP |
| | (+hexapeptide) | CTSEEIDHEDITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNFNSETVPQKSSLEEPYFY |
| | | KTKIKLCILLHAFRIRAVTIDRVMSYLNAS |
| 277 | p35 (D159F) | RNLPVATPDPGMFPCLHHSQNLLRAVSNMLQKARQTLEFYP |
| | (+hexapeptide) | CTSEEIDHEDITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNFNSETVPQKSSLEEPFFY |
| | | KTKIKLCILLHAFRIRAVTIDRVMSYLNAS |
| 278 | p35 (Y161S) | RNLPVATPDPGMFPCLHHSQNLLRAVSNMLQKARQTLEFYP |
| | (+hexapeptide) | CTSEEIDHEDITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNENSETVPQKSSLEEPDFS |
| | | KTKIKLCILLHAFRIRAVTIDRVMSYLNAS |
| 279 | p35 (Y161T) | RNLPVATPDPGMFPCLHHSQNLLRAVSNMLQKARQTLEFYP |
| | (+hexapeptide) | CTSEEIDHEDITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNFNSETVPQKSSLEEPDFT |
| | | KTKIKLCILLHAFRIRAVTIDRVMSYLNAS |
| 280 | p35 (Y161A) | RNLPVATPDPGMFPCLHHSQNLLRAVSNMLQKARQTLEFYP |
| | (+hexapeptide) | CTSEEIDHEDITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNFNSETVPQKSSLEEPDFA |
| | | KTKIKLCILLHAFRIRAVTIDRVMSYLNAS |
| 281 | p35 (Y161W) | RNLPVATPDPGMFPCLHHSQNLLRAVSNMLQKARQTLEFYP |
| | (+hexapeptide) | CTSEEIDHEDITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNFNSETVPQKSSLEEPDFW |
| | | KTKIKLCILLHAFRIRAVTIDRVMSYLNAS |
| 282 | p35 (K162E) | RNLPVATPDPGMFPCLHHSQNLLRAVSNMLQKARQTLEFYP |
| | (+hexapeptide) | CTSEEIDHEDITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNFNSETVPQKSSLEEPDFY |
| | | ETKIKLCILLHAFRIRAVTIDRVMSYLNAS |
| 283 | p35 (K162D) | RNLPVATPDPGMFPCLHHSQNLLRAVSNMLQKARQTLEFYP |
| | (+hexapeptide) | CTSEEIDHEDITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNFNSETVPQKSSLEEPDFY |
| | | DTKIKLCILLHAFRIRAVTIDRVMSYLNAS |
| 284 | p35 (F33G- | RNLPVATPDPGMFPCLHHSQNLLRAVSNMLQKARQTLEGYP |
| | D159K-Y161T) | CTSEEIDHEDITKDKTSTVEACLPLELTKNESCLNSRETSFITN |
| | (+hexapeptide) | GSCLASRKTSFMMALCLSSIYEDLKMYQVEFKTMNAKLLM |
| | | DPKRQIFLDQNMLAVIDELMQALNFNSETVPQKSSLEEPKFT |
| | | KTKIKLCILLHAFRIRAVTIDRVMSYLNAS |
| 285 | p35 (F33K- | RNLPVATPDPGMFPCLHHSQNLLRAVSNMLQKARQTLEKYP |
| | D159K-Y161T) | CTSEEIDHEDITKDKTSTVEACLPLELTKNESCLNSRETSFITN |
| | (+hexapeptide) | GSCLASRKTSFMMALCLSSIYEDLKMYQVEFKTMNAKLLM |
| | | DPKRQIFLDQNMLAVIDELMQALNFNSETVPQKSSLEEPKFT |
| | | KTKIKLCILLHAFRIRAVTIDRVMSYLNAS |
| 286 | p35 (F33S- | RNLPVATPDPGMFPCLHHSQNLLRAVSNMLQKARQTLESYP |
| | D159K-Y161T) | CTSEEIDHEDITKDKTSTVEACLPLELTKNESCLNSRETSFITN |
| | (+hexapeptide) | GSCLASRKTSFMMALCLSSIYEDLKMYQVEFKTMNAKLLM |
| | | DPKRQIFLDQNMLAVIDELMQALNFNSETVPQKSSLEEPKFT |
| | | KTKIKLCILLHAFRIRAVTIDRVMSYLNAS |
| 287 | p35 (Y34G- | RNLPVATPDPGMFPCLHHSQNLLRAVSNMLQKARQTLEFGP |
| | D159K-Y161T) | CTSEEIDHEDITKDKTSTVEACLPLELTKNESCLNSRETSFITN |
| | (+hexapeptide) | GSCLASRKTSFMMALCLSSIYEDLKMYQVEFKTMNAKLLM |
| | | DPKRQIFLDQNMLAVIDELMQALNFNSETVPQKSSLEEPKFT |
| | | KTKIKLCILLHAFRIRAVTIDRVMSYLNAS |
| 288 | p35 (Y34S- | RNLPVATPDPGMFPCLHHSQNLLRAVSNMLQKARQTLEFSP |
| | D159K-Y161T) | CTSEEIDHEDITKDKTSTVEACLPLELTKNESCLNSRETSFITN |
| | (+hexapeptide) | GSCLASRKTSFMMALCLSSIYEDLKMYQVEFKTMNAKLLM |
| | | DPKRQIFLDQNMLAVIDELMQALNFNSETVPQKSSLEEPKFT |
| | | KTKIKLCILLHAFRIRAVTIDRVMSYLNAS |
| 289 | p35 (Y34T- | RNLPVATPDPGMFPCLHHSQNLLRAVSNMLQKARQTLEFTP |
| | D159K-Y161T) | CTSEEIDHEDITKDKTSTVEACLPLELTKNESCLNSRETSFITN |
| | (+hexapeptide) | GSCLASRKTSFMMALCLSSIYEDLKMYQVEFKTMNAKLLM |
| | | DPKRQIFLDQNMLAVIDELMQALNFNSETVPQKSSLEEPKFT |
| | | KTKIKLCILLHAFRIRAVTIDRVMSYLNAS |
| 290 | p35 (P35Q- | RNLPVATPDPGMFPCLHHSQNLLRAVSNMLQKARQTLEFYQ |
| | D159K-Y161T) | CTSEEIDHEDITKDKTSTVEACLPLELTKNESCLNSRETSFITN |
| | (+hexapeptide) | GSCLASRKTSFMMALCLSSIYEDLKMYQVEFKTMNAKLLM |
| | | DPKRQIFLDQNMLAVIDELMQALNFNSETVPQKSSLEEPKFT |
| | | KTKIKLCILLHAFRIRAVTIDRVMSYLNAS |
| 291 | p35 (P35E- | RNLPVATPDPGMFPCLHHSQNLLRAVSNMLQKARQTLEFYE |
| | D159K-Y161T) | CTSEEIDHEDITKDKTSTVEACLPLELTKNESCLNSRETSFITN |
| | (+hexapeptide) | GSCLASRKTSFMMALCLSSIYEDLKMYQVEFKTMNAKLLM |
| | | DPKRQIFLDQNMLAVIDELMQALNFNSETVPQKSSLEEPKFT |
| | | KTKIKLCILLHAFRIRAVTIDRVMSYLNAS |
| 292 | p35 (F33S-Y34T | RNLPVATPDPGMFPCLHHSQNLLRAVSNMLQKARQTLESTP |
| | D159K-Y161T) | CTSEEIDHEDITKDKTSTVEACLPLELTKNESCLNSRETSFITN |
| | (+hexapeptide) | GSCLASRKTSFMMALCLSSIYEDLKMYQVEFKTMNAKLLM |
| | | DPKRQIFLDQNMLAVIDELMQALNFNSETVPQKSSLEEPKFT |
| | | KTKIKLCILLHAFRIRAVTIDRVMSYLNAS |
| 293 | p35 (F33S-Y34S | RNLPVATPDPGMFPCLHHSQNLLRAVSNMLQKARQTLESSP |
| | D159K-Y161T) | CTSEEIDHEDITKDKTSTVEACLPLELTKNESCLNSRETSFITN |
| | (+hexapeptide) | GSCLASRKTSFMMALCLSSIYEDLKMYQVEFKTMNAKLLM |
| | | DPKRQIFLDQNMLAVIDELMQALNFNSETVPQKSSLEEPKFT |
| | | KTKIKLCILLHAFRIRAVTIDRVMSYLNAS |
| 294 | p35 (F33G- | RNLPVATPDPGMFPCLHHSQNLLRAVSNMLQKARQTLEGYP |
| | Y161T) | CTSEEIDHEDITKDKTSTVEACLPLELTKNESCLNSRETSFITN |
| | (+hexapeptide) | GSCLASRKTSFMMALCLSSIYEDLKMYQVEFKTMNAKLLM |
| | | DPKRQIFLDQNMLAVIDELMQALNFNSETVPQKSSLEEPDFT |
| | | KTKIKLCILLHAFRIRAVTIDRVMSYLNAS |
| 295 | p35 (F33K- | RNLPVATPDPGMFPCLHHSQNLLRAVSNMLQKARQTLEKYP |
| | Y161T) | CTSEEIDHEDITKDKTSTVEACLPLELTKNESCLNSRETSFITN |
| | (+hexapeptide) | GSCLASRKTSFMMALCLSSIYEDLKMYQVEFKTMNAKLLM |
| | | DPKRQIFLDQNMLAVIDELMQALNFNSETVPQKSSLEEPDFT |
| | | KTKIKLCILLHAFRIRAVTIDRVMSYLNAS |
| 296 | p35 (F33S-Y161T) | RNLPVATPDPGMFPCLHHSQNLLRAVSNMLQKARQTLESYP |
| | (+hexapeptide) | CTSEEIDHEDITKDKTSTVEACLPLELTKNESCLNSRETSFITN |
| | | GSCLASRKTSFMMALCLSSIYEDLKMYQVEFKTMNAKLLM |
| | | DPKRQIFLDQNMLAVIDELMQALNFNSETVPQKSSLEEPDFT |
| | | KTKIKLCILLHAFRIRAVTIDRVMSYLNAS |
| 297 | p35 (Y34G- | RNLPVATPDPGMFPCLHHSQNLLRAVSNMLQKARQTLEFGP |
| | Y161T) | CTSEEIDHEDITKDKTSTVEACLPLELTKNESCLNSRETSFITN |
| | (+hexapeptide) | GSCLASRKTSFMMALCLSSIYEDLKMYQVEFKTMNAKLLM |
| | | DPKRQIFLDQNMLAVIDELMQALNFNSETVPQKSSLEEPDFT |
| | | KTKIKLCILLHAFRIRAVTIDRVMSYLNAS |
| 298 | p35 (Y34S- | RNLPVATPDPGMFPCLHHSQNLLRAVSNMLQKARQTLEFSP |
| | Y161T) | CTSEEIDHEDITKDKTSTVEACLPLELTKNESCLNSRETSFITN |
| | (+hexapeptide) | GSCLASRKTSFMMALCLSSIYEDLKMYQVEFKTMNAKLLM |
| | | DPKRQIFLDQNMLAVIDELMQALNFNSETVPQKSSLEEPDFT |
| | | KTKIKLCILLHAFRIRAVTIDRVMSYLNAS |
| 299 | p35 (Y34T- | RNLPVATPDPGMFPCLHHSQNLLRAVSNMLQKARQTLEFTP |
| | Y161T) | CTSEEIDHEDITKDKTSTVEACLPLELTKNESCLNSRETSFITN |
| | (+hexapeptide) | GSCLASRKTSFMMALCLSSIYEDLKMYQVEFKTMNAKLLM |
| | | DPKRQIFLDQNMLAVIDELMQALNFNSETVPQKSSLEEPDFT |
| | | KTKIKLCILLHAFRIRAVTIDRVMSYLNAS |
| 300 | p35 (F33S-Y34T- | RNLPVATPDPGMFPCLHHSQNLLRAVSNMLQKARQTLESTP |
| | Y161T) | CTSEEIDHEDITKDKTSTVEACLPLELTKNESCLNSRETSFITN |
| | (+hexapeptide) | GSCLASRKTSFMMALCLSSIYEDLKMYQVEFKTMNAKLLM |
| | | DPKRQIFLDQNMLAVIDELMQALNFNSETVPQKSSLEEPDFT |
| | | KTKIKLCILLHAFRIRAVTIDRVMSYLNASG |
| 301 | p35 (F33S-Y34S- | RNLPVATPDPGMFPCLHHSQNLLRAVSNMLQKARQTLESSP |
| | Y161T) | CTSEEIDHEDITKDKTSTVEACLPLELTKNESCLNSRETSFITN |
| | (+hexapeptide) | GSCLASRKTSFMMALCLSSIYEDLKMYQVEFKTMNAKLLM |
| | | DPKRQIFLDQNMLAVIDELMQALNFNSETVPQKSSLEEPDFT |
| | | KTKIKLCILLHAFRIRAVTIDRVMSYLNAS |
| 302 | p35 (F33S-Y34G- | RNLPVATPDPGMFPCLHHSQNLLRAVSNMLQKARQTLESGP |
| | Y161T) | CTSEEIDHEDITKDKTSTVEACLPLELTKNESCLNSRETSFITN |
| | (+hexapeptide) | GSCLASRKTSFMMALCLSSIYEDLKMYQVEFKTMNAKLLM |
| | | DPKRQIFLDQNMLAVIDELMQALNFNSETVPQKSSLEEPDFT |
| | | KTKIKLCILLHAFRIRAVTIDRVMSYLNAS |
| 303 | p35 (F33G- | RNLPVATPDPGMFPCLHHSQNLLRAVSNMLQKARQTLEGYP |
| | D159K-Y161T) | CTSEEIDHEDITKDKTSTVEACLPLELTKNESCLNSRETSFITN |
| | (+hexapeptide) | GSCLASRKTSFMMALCLSSIYEDLKMYQVEFKTMNAKLLM |
| | | DPKRQIFLDQNMLAVIDELMQALNFNSETVPQKSSLEEPKFT |
| | | KTKIKLCILLHAFRIRAVTIDRVMSYLNAS |
| 304 | p35 (E40K) | RNLPVATPDPGMFPCLHHSQNLLRAVSNMLQKARQTLEFYP |
| | (+hexapeptide)- | CTSEKIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-KIH | DPKRQIFLDQNMLAVIDELMQALNFNSETVPQKSSLEEPDFY |
| | Knob: S354C- | KTKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 305 | p35 (E40R) | RNLPVATPDPGMFPCLHHSQNLLRAVSNMLQKARQTLEFYP |
| | (+hexapeptide)- | CTSERIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-KIH | DPKRQIFLDQNMLAVIDELMQALNFNSETVPQKSSLEEPDFY |
| | Knob: S354C- | KTKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 306 | p35 (E40W) | RNLPVATPDPGMFPCLHHSQNLLRAVSNMLQKARQTLEFYP |
| | (+hexapeptide)- | CTSEWIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-KIH | DPKRQIFLDQNMLAVIDELMQALNFNSETVPQKSSLEEPDFY |
| | Knob: S354C- | KTKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 307 | p35 (I41A) | RNLPVATPDPGMFPCLHHSQNLLRAVSNMLQKARQTLEFYP |
| | (+hexapeptide)- | CTSEEADHEDITKDKTSTVEACLPLELTKNESCLNSRETSFIT |
| | 15GS-Fc | NGSCLASRKTSFMMALCLSSIYEDLKMYQVEFKTMNAKLL |
| | (LALADS-KIH | MDPKRQIFLDQNMLAVIDELMQALNFNSETVPQKSSLEEPDF |
| | Knob: S354C- | YKTKIKLCILLHAFRIRAVTIDRVMSYLNASGGGGSGGGGS |
| | T366W) | GGGGSDKTHTCPPCPAPEAAGGPSVFLFPPKPKDTLMISRTP |
| | | EVTCVVVSVSHEDPEVKFNWYVDGVEVHNAKTKPREEQYN |
| | | STYRVVSVLTVLHQDWLNGKEYKCKVSNKALPAPIEKTISK |
| | | AKGQPREPQVYTLPPCRDELTKNQVSLWCLVKGFYPSDIAV |
| | | EWESNGQPENNYKTTPPVLDSDGSFFLYSKLTVDKSRWQQG |
| | | NVFSCSVMHEALHNHYTQKSLSLSPGK |
| 308 | p35 (I41V) | RNLPVATPDPGMFPCLHHSQNLLRAVSNMLQKARQTLEFYP |
| | (+hexapeptide)- | CTSEEVDHEDITKDKTSTVEACLPLELTKNESCLNSRETSFIT |
| | 15GS-Fc | NGSCLASRKTSFMMALCLSSIYEDLKMYQVEFKTMNAKLL |
| | (LALADS-KIH | MDPKRQIFLDQNMLAVIDELMQALNFNSETVPQKSSLEEPDF |
| | Knob: S354C- | YKTKIKLCILLHAFRIRAVTIDRVMSYLNASGGGGSGGGGS |
| | T366W) | GGGGSDKTHTCPPCPAPEAAGGPSVFLFPPKPKDTLMISRTP |
| | | EVTCVVVSVSHEDPEVKFNWYVDGVEVHNAKTKPREEQYN |
| | | STYRVVSVLTVLHQDWLNGKEYKCKVSNKALPAPIEKTISK |
| | | AKGQPREPQVYTLPPCRDELTKNQVSLWCLVKGFYPSDIAV |
| | | EWESNGQPENNYKTTPPVLDSDGSFFLYSKLTVDKSRWQQG |
| | | NVFSCSVMHEALHNHYTQKSLSLSPGK |
| 309 | p35 (I41S) | RNLPVATPDPGMFPCLHHSQNLLRAVSNMLQKARQTLEFYP |
| | (+hexapeptide)- | CTSEESDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-KIH | DPKRQIFLDQNMLAVIDELMQALNFNSETVPQKSSLEEPDFY |
| | Knob: S354C- | KTKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 310 | p35 (I41T) | RNLPVATPDPGMFPCLHHSQNLLRAVSNMLQKARQTLEFYP |
| | (+hexapeptide)- | CTSEETDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-KIH | DPKRQIFLDQNMLAVIDELMQALNFNSETVPQKSSLEEPDFY |
| | Knob: S354C- | KTKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 311 | p35 (D42A) | RNLPVATPDPGMFPCLHHSQNLLRAVSNMLQKARQTLEFYP |
| | (+hexapeptide)- | CTSEEIAHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-KIH | DPKRQIFLDQNMLAVIDELMQALNFNSETVPQKSSLEEPDFY |
| | Knob: S354C- | KTKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 312 | p35 (D42L) | RNLPVATPDPGMFPCLHHSQNLLRAVSNMLQKARQTLEFYP |
| | (+hexapeptide)- | CTSEEILHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-KIH | DPKRQIFLDQNMLAVIDELMQALNFNSETVPQKSSLEEPDFY |
| | Knob: S354C- | KTKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 313 | p35 (D42W) | RNLPVATPDPGMFPCLHHSQNLLRAVSNMLQKARQTLEFYP |
| | (+hexapeptide)- | CTSEEIWHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-KIH | DPKRQIFLDQNMLAVIDELMQALNFNSETVPQKSSLEEPDFY |
| | Knob: S354C- | KTKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 314 | p35 (E44A) | RNLPVATPDPGMFPCLHHSQNLLRAVSNMLQKARQTLEFYP |
| | (+hexapeptide)- | CTSEEIDHADITKDKTSTVEACLPLELTKNESCLNSRETSFITN |
| | 15GS-Fc | GSCLASRKTSFMMALCLSSIYEDLKMYQVEFKTMNAKLLM |
| | (LALADS-KIH | DPKRQIFLDQNMLAVIDELMQALNFNSETVPQKSSLEEPDFY |
| | Knob: S354C- | KTKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 315 | p35 (E44K) | RNLPVATPDPGMFPCLHHSQNLLRAVSNMLQKARQTLEFYP |
| | (+hexapeptide)- | CTSEEIDHKDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-KIH | DPKRQIFLDQNMLAVIDELMQALNFNSETVPQKSSLEEPDFY |
| | Knob: S354C- | KTKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 316 | p35 (E44R) | RNLPVATPDPGMFPCLHHSQNLLRAVSNMLQKARQTLEFYP |
| | (+hexapeptide)- | CTSEEIDHRDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-KIH | DPKRQIFLDQNMLAVIDELMQALNFNSETVPQKSSLEEPDFY |
| | Knob: S354C- | KTKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 317 | p35 (D159K) | RNLPVATPDPGMFPCLHHSQNLLRAVSNMLQKARQTLEFYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-KIH | DPKRQIFLDQNMLAVIDELMQALNFNSETVPQKSSLEEPKFY |
| | Knob: S354C- | KTKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 318 | p35 (D159R) | RNLPVATPDPGMFPCLHHSQNLLRAVSNMLQKARQTLEFYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-KIH | DPKRQIFLDQNMLAVIDELMQALNFNSETVPQKSSLEEPRFY |
| | Knob: S354C- | KTKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 319 | p35 (D159Y) | RNLPVATPDPGMFPCLHHSQNLLRAVSNMLQKARQTLEFYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-KIH | DPKRQIFLDQNMLAVIDELMQALNFNSETVPQKSSLEEPYFY |
| | Knob: S354C- | KTKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 320 | p35 (D159F) | RNLPVATPDPGMFPCLHHSQNLLRAVSNMLQKARQTLEFYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-KIH | DPKRQIFLDQNMLAVIDELMQALNFNSETVPQKSSLEEPFFY |
| | Knob: S354C- | KTKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 321 | p35 (Y161S) | RNLPVATPDPGMFPCLHHSQNLLRAVSNMLQKARQTLEFYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS)(KIH | DPKRQIFLDQNMLAVIDELMQALNFNSETVPQKSSLEEPDFS |
| | Knob: S354C- | KTKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 322 | p35 (Y161T) | RNLPVATPDPGMFPCLHHSQNLLRAVSNMLQKARQTLEFYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-KIH | DPKRQIFLDQNMLAVIDELMQALNFNSETVPQKSSLEEPDFT |
| | Knob: S354C- | KTKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 323 | p35 (Y161A) | RNLPVATPDPGMFPCLHHSQNLLRAVSNMLQKARQTLEFYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-KIH | DPKRQIFLDQNMLAVIDELMQALNFNSETVPQKSSLEEPDFA |
| | Knob: S354C- | KTKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 324 | p35 (Y161W) | RNLPVATPDPGMFPCLHHSQNLLRAVSNMLQKARQTLEFYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-KIH | DPKRQIFLDQNMLAVIDELMQALNFNSETVPQKSSLEEPDFW |
| | Knob: S354C- | KTKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 325 | p35 (K162E) | RNLPVATPDPGMFPCLHHSQNLLRAVSNMLQKARQTLEFYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-KIH | DPKRQIFLDQNMLAVIDELMQALNFNSETVPQKSSLEEPDFY |
| | Knob: S354C- | ETKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 326 | p35 (K162D) | RNLPVATPDPGMFPCLHHSQNLLRAVSNMLQKARQTLEFYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-KIH | DPKRQIFLDQNMLAVIDELMQALNFNSETVPQKSSLEEPDFY |
| | Knob: S354C- | DTKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 327 | p35 (D159K- | RNLPVATPDPGMFPCLHHSQNLLRAVSNMLQKARQTLEFYP |
| | Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-KIH | KTKIKLCILLHAFRIRAVTIDRVMSYLNASGGGGSGGGGSG |
| | Knob: S354C- | GGGSDKTHTCPPCPAPEAAGGPSVFLFPPKPKDTLMISRTPE |
| | T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 328 | p35 (F33G- | RNLPVATPDPGMFPCLHHSQNLLRAVSNMLQKARQTLEGYP |
| | D159K-Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-KIH | KTKIKLCILLHAFRIRAVTIDRVMSYLNASGGGGSGGGGSG |
| | Knob: S354C- | GGGSDKTHTCPPCPAPEAAGGPSVFLFPPKPKDTLMISRTPE |
| | T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 329 | p35 (F33K- | RNLPVATPDPGMFPCLHHSQNLLRAVSNMLQKARQTLEKYP |
| | D159K-Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-KIH | KTKIKLCILLHAFRIRAVTIDRVMSYLNASGGGGSGGGGSG |
| | Knob: S354C- | GGGSDKTHTCPPCPAPEAAGGPSVFLFPPKPKDTLMISRTPE |
| | T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 330 | p35 (F33S- | RNLPVATPDPGMFPCLHHSQNLLRAVSNMLQKARQTLESYP |
| | D159K-Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-KIH | KTKIKLCILLHAFRIRAVTIDRVMSYLNASGGGGSGGGGSG |
| | Knob: S354C- | GGGSDKTHTCPPCPAPEAAGGPSVFLFPPKPKDTLMISRTPE |
| | T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 331 | p35 (Y34G- | RNLPVATPDPGMFPCLHHSQNLLRAVSNMLQKARQTLEFGP |
| | D159K-Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-KIH | KTKIKLCILLHAFRIRAVTIDRVMSYLNASGGGGSGGGGSG |
| | Knob: S354C- | GGGSDKTHTCPPCPAPEAAGGPSVFLFPPKPKDTLMISRTPE |
| | T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 332 | p35 (Y34S- | RNLPVATPDPGMFPCLHHSQNLLRAVSNMLQKARQTLEFSP |
| | D159K-Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-KIH | KTKIKLCILLHAFRIRAVTIDRVMSYLNASGGGGSGGGGSG |
| | Knob: S354C- | GGGSDKTHTCPPCPAPEAAGGPSVFLFPPKPKDTLMISRTPE |
| | T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 333 | p35 (Y34T- | RNLPVATPDPGMFPCLHHSQNLLRAVSNMLQKARQTLEFTP |
| | D159K-Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-KIH | KTKIKLCILLHAFRIRAVTIDRVMSYLNASGGGGSGGGGSG |
| | Knob: S354C- | GGGSDKTHTCPPCPAPEAAGGPSVFLFPPKPKDTLMISRTPE |
| | T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 334 | p35 (P35Q- | RNLPVATPDPGMFPCLHHSQNLLRAVSNMLQKARQTLEFYQ |
| | D159K-Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-KIH | KTKIKLCILLHAFRIRAVTIDRVMSYLNASGGGGSGGGGSG |
| | Knob: S354C- | GGGSDKTHTCPPCPAPEAAGGPSVFLFPPKPKDTLMISRTPE |
| | T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 335 | p35 (P35E- | RNLPVATPDPGMFPCLHHSQNLLRAVSNMLQKARQTLEFYE |
| | D159K-Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-KIH | KTKIKLCILLHAFRIRAVTIDRVMSYLNASGGGGSGGGGSG |
| | Knob: S354C- | GGGSDKTHTCPPCPAPEAAGGPSVFLFPPKPKDTLMISRTPE |
| | T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 336 | p35 (F33S-Y34T | RNLPVATPDPGMFPCLHHSQNLLRAVSNMLQKARQTLESTP |
| | D159K-Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-KIH | KTKIKLCILLHAFRIRAVTIDRVMSYLNASGGGGSGGGGSG |
| | Knob: S354C- | GGGSDKTHTCPPCPAPEAAGGPSVFLFPPKPKDTLMISRTPE |
| | T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 337 | p35 (F33S-Y34S | RNLPVATPDPGMFPCLHHSQNLLRAVSNMLQKARQTLESSP |
| | D159K-Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-KIH | KTKIKLCILLHAFRIRAVTIDRVMSYLNASGGGGSGGGGSG |
| | Knob: S354C- | GGGSDKTHTCPPCPAPEAAGGPSVFLFPPKPKDTLMISRTPE |
| | T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 338 | p35 (F33G- | RNLPVATPDPGMFPCLHHSQNLLRAVSNMLQKARQTLEGYP |
| | Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPDFT |
| | (LALADS-KIH | KTKIKLCILLHAFRIRAVTIDRVMSYLNASGGGGSGGGGSG |
| | Knob: S354C- | GGGSDKTHTCPPCPAPEAAGGPSVFLFPPKPKDTLMISRTPE |
| | T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 339 | p35 (F33K- | RNLPVATPDPGMFPCLHHSQNLLRAVSNMLQKARQTLEKYP |
| | Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPDFT |
| | (LALADS-KIH | KTKIKLCILLHAFRIRAVTIDRVMSYLNASGGGGSGGGGSG |
| | Knob: S354C- | GGGSDKTHTCPPCPAPEAAGGPSVFLFPPKPKDTLMISRTPE |
| | T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 340 | p35 (F33S-Y161T) | RNLPVATPDPGMFPCLHHSQNLLRAVSNMLQKARQTLESYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-KIH | DPKRQIFLDQNMLAVIDELMQALNFNSETVPQKSSLEEPDFT |
| | Knob: S354C- | KTKIKLCILLHAFRIRAVTIDRVMSYLNASGGGGSGGGGSG |
| | T366W) | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 341 | p35 (Y34G- | RNLPVATPDPGMFPCLHHSQNLLRAVSNMLQKARQTLEFGP |
| | Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPDFT |
| | (LALADS-KIH | KTKIKLCILLHAFRIRAVTIDRVMSYLNASGGGGSGGGGSG |
| | Knob: S354C- | GGGSDKTHTCPPCPAPEAAGGPSVFLFPPKPKDTLMISRTPE |
| | T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 342 | p35 (Y34S- | RNLPVATPDPGMFPCLHHSQNLLRAVSNMLQKARQTLEFSP |
| | Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPDFT |
| | (LALADS-KIH | KTKIKLCILLHAFRIRAVTIDRVMSYLNASGGGGSGGGGSG |
| | Knob: S354C- | GGGSDKTHTCPPCPAPEAAGGPSVFLFPPKPKDTLMISRTPE |
| | T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 343 | p35 (Y34T- | RNLPVATPDPGMFPCLHHSQNLLRAVSNMLQKARQTLEFTP |
| | Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPDFT |
| | (LALADS-KIH | KTKIKLCILLHAFRIRAVTIDRVMSYLNASGGGGSGGGGSG |
| | Knob: S354C- | GGGSDKTHTCPPCPAPEAAGGPSVFLFPPKPKDTLMISRTPE |
| | T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 344 | p35 (F33S-Y34T- | RNLPVATPDPGMFPCLHHSQNLLRAVSNMLQKARQTLESTP |
| | Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPDFT |
| | (LALADS-KIH | KTKIKLCILLHAFRIRAVTIDRVMSYLNASGGGGGSGGGGS |
| | Knob: S354C- | GGGGSDKTHTCPPCPAPEAAGGPSVFLFPPKPKDTLMISRTP |
| | T366W) | EVTCVVVSVSHEDPEVKFNWYVDGVEVHNAKTKPREEQYN |
| | | STYRVVSVLTVLHQDWLNGKEYKCKVSNKALPAPIEKTISK |
| | | AKGQPREPQVYTLPPCRDELTKNQVSLWCLVKGFYPSDIAV |
| | | EWESNGQPENNYKTTPPVLDSDGSFFLYSKLTVDKSRWQQG |
| | | NVFSCSVMHEALHNHYTQKSLSLSPGK |
| 345 | p35 (F33S-Y34S- | RNLPVATPDPGMFPCLHHSQNLLRAVSNMLQKARQTLESSP |
| | Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNENSETVPQKSSLEEPDFT |
| | (LALADS-KIH | KTKIKLCILLHAFRIRAVTIDRVMSYLNASGGGGSGGGGSG |
| | Knob: S354C- | GGGSDKTHTCPPCPAPEAAGGPSVFLFPPKPKDTLMISRTPE |
| | T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 346 | p35 (F33S-Y34G- | RNLPVATPDPGMFPCLHHSQNLLRAVSNMLQKARQTLESGP |
| | Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPDFT |
| | (LALADS-KIH | KTKIKLCILLHAFRIRAVTIDRVMSYLNASGGGGSGGGGSG |
| | Knob: S354C- | GGGSDKTHTCPPCPAPEAAGGPSVFLFPPKPKDTLMISRTPE |
| | T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 347 | p35 (E40K) | RNLPVATPDPGMFPCLHHSQNLLRAVSNMLQKARQTLEFYP |
| | (+hexapeptide)- | CTSEKIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNFNSETVPQKSSLEEPDFY |
| | KIH Knob: | KTKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 348 | p35 (E40R) | RNLPVATPDPGMFPCLHHSQNLLRAVSNMLQKARQTLEFYP |
| | (+hexapeptide)- | CTSERIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNFNSETVPQKSSLEEPDFY |
| | KIH Knob: | KTKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 349 | p35 (E40W) | RNLPVATPDPGMFPCLHHSQNLLRAVSNMLQKARQTLEFYP |
| | (+hexapeptide)- | CTSEWIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNFNSETVPQKSSLEEPDFY |
| | KIH Knob: | KTKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 350 | p35 (I41A) | RNLPVATPDPGMFPCLHHSQNLLRAVSNMLQKARQTLEFYP |
| | (+hexapeptide)- | CTSEEADHEDITKDKTSTVEACLPLELTKNESCLNSRETSFIT |
| | 15GS-Fc | NGSCLASRKTSFMMALCLSSIYEDLKMYQVEFKTMNAKLL |
| | (LALADS-YTE- | MDPKRQIFLDQNMLAVIDELMQALNFNSETVPQKSSLEEPDF |
| | KIH Knob: | YKTKIKLCILLHAFRIRAVTIDRVMSYLNASGGGGSGGGGS |
| | S354C-T366W) | GGGGSDKTHTCPPCPAPEAAGGPSVFLFPPKPKDTLYITREP |
| | | EVTCVVVSVSHEDPEVKFNWYVDGVEVHNAKTKPREEQYN |
| | | STYRVVSVLTVLHQDWLNGKEYKCKVSNKALPAPIEKTISK |
| | | AKGQPREPQVYTLPPCRDELTKNQVSLWCLVKGFYPSDIAV |
| | | EWESNGQPENNYKTTPPVLDSDGSFFLYSKLTVDKSRWQQG |
| | | NVFSCSVMHEALHNHYTQKSLSLSPGK |
| 351 | p35 (I41V) | RNLPVATPDPGMFPCLHHSQNLLRAVSNMLQKARQTLEFYP |
| | (+hexapeptide)- | CTSEEVDHEDITKDKTSTVEACLPLELTKNESCLNSRETSFIT |
| | 15GS-Fc | NGSCLASRKTSFMMALCLSSIYEDLKMYQVEFKTMNAKLL |
| | (LALADS-YTE- | MDPKRQIFLDQNMLAVIDELMQALNFNSETVPQKSSLEEPDF |
| | KIH Knob: | YKTKIKLCILLHAFRIRAVTIDRVMSYLNASGGGGSGGGGS |
| | S354C-T366W) | GGGGSDKTHTCPPCPAPEAAGGPSVFLFPPKPKDTLYITREP |
| | | EVTCVVVSVSHEDPEVKFNWYVDGVEVHNAKTKPREEQYN |
| | | STYRVVSVLTVLHQDWLNGKEYKCKVSNKALPAPIEKTISK |
| | | AKGQPREPQVYTLPPCRDELTKNQVSLWCLVKGFYPSDIAV |
| | | EWESNGQPENNYKTTPPVLDSDGSFFLYSKLTVDKSRWQQG |
| | | NVFSCSVMHEALHNHYTQKSLSLSPGK |
| 352 | p35 (I41S) | RNLPVATPDPGMFPCLHHSQNLLRAVSNMLQKARQTLEFYP |
| | (+hexapeptide)- | CTSEESDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNFNSETVPQKSSLEEPDFY |
| | KIH Knob: | KTKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 353 | p35 (I41T) | RNLPVATPDPGMFPCLHHSQNLLRAVSNMLQKARQTLEFYP |
| | (+hexapeptide)- | CTSEETDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNENSETVPQKSSLEEPDFY |
| | KIH Knob: | KTKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 354 | p35 (D42A) | RNLPVATPDPGMFPCLHHSQNLLRAVSNMLQKARQTLEFYP |
| | (+hexapeptide)- | CTSEEIAHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNFNSETVPQKSSLEEPDFY |
| | KIH Knob: | KTKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 355 | p35 (D42L) | RNLPVATPDPGMFPCLHHSQNLLRAVSNMLQKARQTLEFYP |
| | (+hexapeptide)- | CTSEEILHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNFNSETVPQKSSLEEPDFY |
| | KIH Knob: | KTKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 356 | p35 (D42W) | RNLPVATPDPGMFPCLHHSQNLLRAVSNMLQKARQTLEFYP |
| | (+hexapeptide)- | CTSEEIWHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNFNSETVPQKSSLEEPDFY |
| | KIH Knob: | KTKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 357 | p35 (E44A) | RNLPVATPDPGMFPCLHHSQNLLRAVSNMLQKARQTLEFYP |
| | (+hexapeptide)- | CTSEEIDHADITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNFNSETVPQKSSLEEPDFY |
| | KIH Knob: | KTKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 358 | p35 (E44K) | RNLPVATPDPGMFPCLHHSQNLLRAVSNMLQKARQTLEFYP |
| | (+hexapeptide)- | CTSEEIDHKDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNFNSETVPQKSSLEEPDFY |
| | KIH Knob: | KTKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 359 | p35 (E44R) | RNLPVATPDPGMFPCLHHSQNLLRAVSNMLQKARQTLEFYP |
| | (+hexapeptide)- | CTSEEIDHRDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNFNSETVPQKSSLEEPDFY |
| | KIH Knob: | KTKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 360 | p35 (D159K) | RNLPVATPDPGMFPCLHHSQNLLRAVSNMLQKARQTLEFYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNFNSETVPQKSSLEEPKFY |
| | KIH Knob: | KTKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 361 | p35 (D159R) | RNLPVATPDPGMFPCLHHSQNLLRAVSNMLQKARQTLEFYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNFNSETVPQKSSLEEPRFY |
| | KIH Knob: | KTKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 362 | p35 (D159Y) | RNLPVATPDPGMFPCLHHSQNLLRAVSNMLQKARQTLEFYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNFNSETVPQKSSLEEPYFY |
| | KIH Knob: | KTKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 363 | p35 (D159F) | RNLPVATPDPGMFPCLHHSQNLLRAVSNMLQKARQTLEFYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNFNSETVPQKSSLEEPFFY |
| | KIH Knob: | KTKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 364 | p35 (Y161S) | RNLPVATPDPGMFPCLHHSQNLLRAVSNMLQKARQTLEFYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNFNSETVPQKSSLEEPDFS |
| | KIH Knob: | KTKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 365 | p35(Y161T) | RNLPVATPDPGMFPCLHHSQNLLRAVSNMLQKARQTLEFYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNFNSETVPQKSSLEEPDFT |
| | KIH Knob: | KTKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 366 | p35 (Y161A) | RNLPVATPDPGMFPCLHHSQNLLRAVSNMLQKARQTLEFYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNFNSETVPQKSSLEEPDFA |
| | KIH Knob: | KTKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 367 | p35(Y161W) | RNLPVATPDPGMFPCLHHSQNLLRAVSNMLQKARQTLEFYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNFNSETVPQKSSLEEPDFW |
| | KIH Knob: | KTKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 368 | p35 (K162E) | RNLPVATPDPGMFPCLHHSQNLLRAVSNMLQKARQTLEFYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNFNSETVPQKSSLEEPDFY |
| | KIH Knob: | ETKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 369 | p35 (K162D) | RNLPVATPDPGMFPCLHHSQNLLRAVSNMLQKARQTLEFYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNFNSETVPQKSSLEEPDFY |
| | KIH Knob: | DTKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 370 | p35 (D159K- | RNLPVATPDPGMFPCLHHSQNLLRAVSNMLQKARQTLEFYP |
| | Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-YTE- | KTKIKLCILLHAFRIRAVTIDRVMSYLNASGGGGSGGGGSG |
| | KIH Knob: | GGGSDKTHTCPPCPAPEAAGGPSVFLFPPKPKDTLYITREPE |
| | S354C-T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 371 | p35 (F33G- | RNLPVATPDPGMFPCLHHSQNLLRAVSNMLQKARQTLEGYP |
| | D159K-Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-YTE- | KTKIKLCILLHAFRIRAVTIDRVMSYLNASGGGGSGGGGSG |
| | KIH Knob: | GGGSDKTHTCPPCPAPEAAGGPSVFLFPPKPKDTLYITREPE |
| | S354C-T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 372 | p35 (F33K- | RNLPVATPDPGMFPCLHHSQNLLRAVSNMLQKARQTLEKYP |
| | D159K-Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-YTE- | KTKIKLCILLHAFRIRAVTIDRVMSYLNASGGGGSGGGGSG |
| | KIH Knob: | GGGSDKTHTCPPCPAPEAAGGPSVFLFPPKPKDTLYITREPE |
| | S354C-T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 373 | p35 (F33S- | RNLPVATPDPGMFPCLHHSQNLLRAVSNMLQKARQTLESYP |
| | D159K-Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-YTE- | KTKIKLCILLHAFRIRAVTIDRVMSYLNASGGGGSGGGGSG |
| | KIH Knob: | GGGSDKTHTCPPCPAPEAAGGPSVFLFPPKPKDTLMISRTPE |
| | S354C-T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 374 | p35 (Y34G- | RNLPVATPDPGMFPCLHHSQNLLRAVSNMLQKARQTLEFGP |
| | D159K-Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-YTE- | KTKIKLCILLHAFRIRAVTIDRVMSYLNASGGGGSGGGGSG |
| | KIH Knob: | GGGSDKTHTCPPCPAPEAAGGPSVFLFPPKPKDTLYITREPE |
| | S354C-T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 375 | p35 (Y34S- | RNLPVATPDPGMFPCLHHSQNLLRAVSNMLQKARQTLEFSP |
| | D159K-Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-YTE- | KTKIKLCILLHAFRIRAVTIDRVMSYLNASGGGGSGGGGSG |
| | KIH Knob: | GGGSDKTHTCPPCPAPEAAGGPSVFLFPPKPKDTLYITREPE |
| | S354C-T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 376 | p35 (Y34T- | RNLPVATPDPGMFPCLHHSQNLLRAVSNMLQKARQTLEFTP |
| | D159K-Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-YTE- | KTKIKLCILLHAFRIRAVTIDRVMSYLNASGGGGSGGGGSG |
| | KIH Knob: | GGGSDKTHTCPPCPAPEAAGGPSVFLFPPKPKDTLYITREPE |
| | S354C-T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 377 | p35 (P35Q- | RNLPVATPDPGMFPCLHHSQNLLRAVSNMLQKARQTLEFYQ |
| | D159K-Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-YTE- | KTKIKLCILLHAFRIRAVTIDRVMSYLNASGGGGSGGGGSG |
| | KIH Knob: | GGGSDKTHTCPPCPAPEAAGGPSVFLFPPKPKDTLYITREPE |
| | S354C-T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 378 | p35 (P35E- | RNLPVATPDPGMFPCLHHSQNLLRAVSNMLQKARQTLEFYE |
| | D159K-Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-YTE- | KTKIKLCILLHAFRIRAVTIDRVMSYLNASGGGGSGGGGSG |
| | KIH Knob: | GGGSDKTHTCPPCPAPEAAGGPSVFLFPPKPKDTLYITREPE |
| | S354C-T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 379 | p35 (F33S-Y34T | RNLPVATPDPGMFPCLHHSQNLLRAVSNMLQKARQTLESTP |
| | D159K-Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-YTE- | KTKIKLCILLHAFRIRAVTIDRVMSYLNASGGGGSGGGGSG |
| | KIH Knob: | GGGSDKTHTCPPCPAPEAAGGPSVFLFPPKPKDTLYITREPE |
| | S354C-T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 380 | p35 (F33S-Y34S | RNLPVATPDPGMFPCLHHSQNLLRAVSNMLQKARQTLESSP |
| | D159K-Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPKFT |
| | (LALADS-YTE- | KTKIKLCILLHAFRIRAVTIDRVMSYLNASGGGGSGGGGSG |
| | KIH Knob: | GGGSDKTHTCPPCPAPEAAGGPSVFLFPPKPKDTLYITREPE |
| | S354C-T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 381 | p35 (F33G- | RNLPVATPDPGMFPCLHHSQNLLRAVSNMLQKARQTLEGYP |
| | Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPDFT |
| | (LALADS-YTE- | KTKIKLCILLHAFRIRAVTIDRVMSYLNASGGGGSGGGGSG |
| | KIH Knob: | GGGSDKTHTCPPCPAPEAAGGPSVFLFPPKPKDTLYITREPE |
| | S354C-T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 382 | p35 (F33K- | RNLPVATPDPGMFPCLHHSQNLLRAVSNMLQKARQTLEKYP |
| | Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPDFT |
| | (LALADS-YTE- | KTKIKLCILLHAFRIRAVTIDRVMSYLNASGGGGSGGGGSG |
| | KIH Knob: | GGGSDKTHTCPPCPAPEAAGGPSVFLFPPKPKDTLYITREPE |
| | S354C-T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 383 | p35 (F33S-Y161T) | RNLPVATPDPGMFPCLHHSQNLLRAVSNMLQKARQTLESYP |
| | (+hexapeptide)- | CTSEEIDHEDITKDKTSTVEACLPLELTKNESCLNSRETSFITN |
| | 15GS-Fc | GSCLASRKTSFMMALCLSSIYEDLKMYQVEFKTMNAKLLM |
| | (LALADS-YTE- | DPKRQIFLDQNMLAVIDELMQALNFNSETVPQKSSLEEPDFT |
| | KIH Knob: | KTKIKLCILLHAFRIRAVTIDRVMSYLNASGGGGSGGGGSG |
| | S354C-T366W) | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 384 | p35 (Y34G- | RNLPVATPDPGMFPCLHHSQNLLRAVSNMLQKARQTLEFGP |
| | Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPDFT |
| | (LALADS-YTE- | KTKIKLCILLHAFRIRAVTIDRVMSYLNASGGGGSGGGGSG |
| | KIH Knob: | GGGSDKTHTCPPCPAPEAAGGPSVFLFPPKPKDTLYITREPE |
| | S354C-T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 385 | p35 (Y34S- | RNLPVATPDPGMFPCLHHSQNLLRAVSNMLQKARQTLEFSP |
| | Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPDFT |
| | (LALADS-YTE- | KTKIKLCILLHAFRIRAVTIDRVMSYLNASGGGGSGGGGSG |
| | KIH Knob: | GGGSDKTHTCPPCPAPEAAGGPSVFLFPPKPKDTLYITREPE |
| | S354C-T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 386 | p35 (Y34T- | RNLPVATPDPGMFPCLHHSQNLLRAVSNMLQKARQTLEFTP |
| | Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPDFT |
| | (LALADS-YTE- | KTKIKLCILLHAFRIRAVTIDRVMSYLNASGGGGSGGGGSG |
| | KIH Knob: | GGGSDKTHTCPPCPAPEAAGGPSVFLFPPKPKDTLYITREPE |
| | S354C-T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 387 | p35 (F33S-Y34T- | RNLPVATPDPGMFPCLHHSQNLLRAVSNMLQKARQTLESTP |
| | Y161T) | CTSEEIDHEDITKDKTSTVEACLPLELTKNESCLNSRETSFITN |
| | (+hexapeptide)- | GSCLASRKTSFMMALCLSSIYEDLKMYQVEFKTMNAKLLM |
| | 15GS-Fc | DPKRQIFLDQNMLAVIDELMQALNFNSETVPQKSSLEEPDFT |
| | (LALADS-YTE- | KTKIKLCILLHAFRIRAVTIDRVMSYLNASGGGGSGGGGSG |
| | KIH Knob: | GGGSDKTHTCPPCPAPEAAGGPSVFLFPPKPKDTLYITREPE |
| | S354C-T366W) | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 388 | p35(+hexapeptide)- | RNLPVATPDPGMFPCLHHSQNLLRAVSNMLQKARQTLEFYP |
| | 15GS-Fc | CTSEEIDHEDITKDKTSTVEACLPLELTKNESCLNSRETSFITN |
| | (LALADS-KIH | GSCLASRKTSFMMALCLSSIYEDLKMYQVEFKTMNAKLLM |
| | Knob: S354C- | DPKRQIFLDQNMLAVIDELMQALNFNSETVPQKSSLEEPDFY |
| | T366W) | KTKIKLCILLHAFRIRAVTIDRVMSYLNASGGGGSGGGGSG |
| | | GGGSDKTHTCPPCPAPEAAGGPSVFLFPPKPKDTLMISRTPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 389 | P35(+hexapeptide)- | RNLPVATPDPGMFPCLHHSQNLLRAVSNMLQKARQTLEFYP |
| | 15GS-Fc | CTSEEIDHEDITKDKTSTVEACLPLELTKNESCLNSRETSFITN |
| | (LALADS-YTE- | GSCLASRKTSFMMALCLSSIYEDLKMYQVEFKTMNAKLLM |
| | KIH Knob: | DPKRQIFLDQNMLAVIDELMQALNFNSETVPQKSSLEEPDFY |
| | S354C-T366W) | KTKIKLCILLHAFRIRAVTIDRVMSYLNASGGGGSGGGGSG |
| | | GGGSDKTHTCPPCPAPEAAGGPSVFLFPPKPKDTLYITREPE |
| | | VTCVVVSVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 390 | IL-12p35 N- | RNLP |
| | terminal | |
| | tetrapeptide | |
| 391 | IL-12p35 N- | RNLPV |
| | terminal | |
| | pentapeptide | |
| 392 | IL-12p35 N- | RNLPVAT |
| | terminal | |
| | septapeptide | |
| 393 | IL-12p35 N- | RNLPVATP |
| | terminal | |
| | octapeptide | |
| 394 | IL-12p35 N- | RNLPVATPD |
| | terminal | |
| | nonapeptide | |
| 395 | IL-12p35 N- | RNLPVATPDP |
| | terminal | |
| | decapeptide | |
| 396 | Fc (LALADS-LS- | DKTHTCPPCPAPEAAGGPSVFLFPPKPKDTLMISRTPEVTCVV |
| | KIH Knob: | VSVSHEDPEVKFNWYVDGVEVHNAKTKPREEQYNSTYRVV |
| | S354C-T366W) | SVLTVLHQDWLNGKEYKCKVSNKALPAPIEKTISKAKGQPR |
| | | EPQVYTLPPCRDELTKNQVSLWCLVKGFYPSDIAVEWESNG |
| | | QPENNYKTTPPVLDSDGSFFLYSKLTVDKSRWQQGNVFSCS |
| | | VLHEALHSHYTQKSLSLSPGK |
| 397 | Fc (LALADS-LS- | DKTHTCPPCPAPEAAGGPSVFLFPPKPKDTLMISRTPEVTCVV |
| | KIH Hole: Y349C- | VSVSHEDPEVKFNWYVDGVEVHNAKTKPREEQYNSTYRVV |
| | T366S-L368A- | SVLTVLHQDWLNGKEYKCKVSNKALPAPIEKTISKAKGQPR |
| | Y407V) | EPQVCTLPPSRDELTKNQVSLSCAVKGFYPSDIAVEWESNGQ |
| | | PENNYKTTPPVLDSDGSFFLVSKLTVDKSRWQQGNVFSCSVL |
| | | HEALHSHYTQKSLSLSPGK |
| 398 | Fc (LALADS-LS- | DKTHTCPPCPAPEAAGGPSVFLFPPKPKDTLMISRTPEVTCVV |
| | KIH Knob: | VSVSHEDPEVKFNWYVDGVEVHNAKTKPREEQYNSTYRVV |
| | K409W) | SVLTVLHQDWLNGKEYKCKVSNKALPAPIEKTISKAKGQPR |
| | | EPQVYTLPPSRDELTKNQVSLTCLVKGFYPSDIAVEWESNGQ |
| | | PENNYKTTPPVLDSDGSFFLYSWLTVDKSRWQQGNVFSCSV |
| | | LHEALHSHYTQKSLSLSPGK |
| 399 | Fc (LALADS-LS- | DKTHTCPPCPAPEAAGGPSVFLFPPKPKDTLMISRTPEVTCVV |
| | KIH Hole: | VSVSHEDPEVKFNWYVDGVEVHNAKTKPREEQYNSTYRVV |
| | D399V-F405T) | SVLTVLHQDWLNGKEYKCKVSNKALPAPIEKTISKAKGQPR |
| | | EPQVYTLPPSRDELTKNQVSLTCLVKGFYPSDIAVEWESNGQ |
| | | PENNYKTTPPVLVSDGSFTLYSKLTVDKSRWQQGNVFSCSV |
| | | LHEALHSHYTQKSLSLSPGK |
| 400 | Fc (LALADS-LS- | DKTHTCPPCPAPEAAGGPSVFLFPPKPKDTLMISRTPEVTCVV |
| | KIH Knob: | VSVSHEDPEVKFNWYVDGVEVHNAKTKPREEQYNSTYRVV |
| | E357W) | SVLTVLHQDWLNGKEYKCKVSNKALPAPIEKTISKAKGQPR |
| | | EPQVYTLPPSRDWLTKNQVSLTCLVKGFYPSDIAVEWESNG |
| | | QPENNYKTTPPVLDSDGSFFLYSKLTVDKSRWQQGNVFSCS |
| | | VLHEALHSHYTQKSLSLSPGK |
| 401 | Fc (LALADS-LS- | DKTHTCPPCPAPEAAGGPSVFLFPPKPKDTLMISRTPEVTCVV |
| | KIH Hole: Y349S) | VSVSHEDPEVKFNWYVDGVEVHNAKTKPREEQYNSTYRVV |
| | | SVLTVLHQDWLNGKEYKCKVSNKALPAPIEKTISKAKGQPR |
| | | EPQVSTLPPSRDELTKNQVSLTCLVKGFYPSDIAVEWESNGQ |
| | | PENNYKTTPPVLDSDGSFFLYSKLTVDKSRWQQGNVFSCSVL |
| | | HEALHSHYTQKSLSLSPGK |
| 402 | Fc (LALADS- | DKTHTCPPCPAPEAAGGPSVFLFPPKPKDTLMISRTPEVTCVV |
| | DHS-KIH Knob: | VSVSHEDPEVKFNWYVDGVEVHNAKTKPREEQYNSTYRVV |
| | S354C-T366W) | SVLTVDHHDWLNGKEYKCKVSNKALPAPIEKTISKAKGQPR |
| | | EPQVYTLPPCRDELTKNQVSLWCLVKGFYPSDIAVEWESNG |
| | | QPENNYKTTPPVLDSDGSFFLYSKLTVDKSRWQQGNVFSCS |
| | | VMHEALHSHYTQKSLSLSPGK |
| 403 | Fc (LALADS- | DKTHTCPPCPAPEAAGGPSVFLFPPKPKDTLMISRTPEVTCVV |
| | DHS-KIH Hole: | VSVSHEDPEVKFNWYVDGVEVHNAKTKPREEQYNSTYRVV |
| | Y349C-T366S- | SVLTVDHHDWLNGKEYKCKVSNKALPAPIEKTISKAKGQPR |
| | L368A-Y407V) | EPQVCTLPPSRDELTKNQVSLSCAVKGFYPSDIAVEWESNGQ |
| | | PENNYKTTPPVLDSDGSFFLVSKLTVDKSRWQQGNVFSCSV |
| | | MHEALHSHYTQKSLSLSPGK |
| 404 | Fc (LALADS- | DKTHTCPPCPAPEAAGGPSVFLFPPKPKDTLMISRTPEVTCVV |
| | DHS-KIH Knob: | VSVSHEDPEVKFNWYVDGVEVHNAKTKPREEQYNSTYRVV |
| | K409W) | SVLTVDHHDWLNGKEYKCKVSNKALPAPIEKTISKAKGQPR |
| | | EPQVYTLPPSRDELTKNQVSLTCLVKGFYPSDIAVEWESNGQ |
| | | PENNYKTTPPVLDSDGSFFLYSWLTVDKSRWQQGNVFSCSV |
| | | MHEALHSHYTQKSLSLSPGK |
| 405 | Fc (LALADS- | DKTHTCPPCPAPEAAGGPSVFLFPPKPKDTLMISRTPEVTCVV |
| | DHS-KIH Hole: | VSVSHEDPEVKFNWYVDGVEVHNAKTKPREEQYNSTYRVV |
| | D399V-F405T) | SVLTVDHHDWLNGKEYKCKVSNKALPAPIEKTISKAKGQPR |
| | | EPQVYTLPPSRDELTKNQVSLTCLVKGFYPSDIAVEWESNGQ |
| | | PENNYKTTPPVLVSDGSFTLYSKLTVDKSRWQQGNVFSCSV |
| | | MHEALHSHYTQKSLSLSPGK |
| 406 | Fc (LALADS- | DKTHTCPPCPAPEAAGGPSVFLFPPKPKDTLMISRTPEVTCVV |
| | DHS-KIH Knob: | VSVSHEDPEVKFNWYVDGVEVHNAKTKPREEQYNSTYRVV |
| | E357W) | SVLTVDHHDWLNGKEYKCKVSNKALPAPIEKTISKAKGQPR |
| | | EPQVYTLPPSRDWLTKNQVSLTCLVKGFYPSDIAVEWESNG |
| | | QPENNYKTTPPVLDSDGSFFLYSKLTVDKSRWQQGNVFSCS |
| | | VMHEALHSHYTQKSLSLSPGK |
| 407 | Fc (LALADS- | DKTHTCPPCPAPEAAGGPSVFLFPPKPKDTLMISRTPEVTCVV |
| | DHS-KIH Hole: | VSVSHEDPEVKFNWYVDGVEVHNAKTKPREEQYNSTYRVV |
| | Y349S) | SVLTVDHHDWLNGKEYKCKVSNKALPAPIEKTISKAKGQPR |
| | | EPQVSTLPPSRDELTKNQVSLTCLVKGFYPSDIAVEWESNGQ |
| | | PENNYKTTPPVLDSDGSFFLYSKLTVDKSRWQQGNVFSCSV |
| | | MHEALHSHYTQKSLSLSPGK |
| 408 | Fc (LALADS-KF- | DKTHTCPPCPAPEAAGGPSVFLFPPKPKDTLMISRTPEVTCVV |
| | KIH Knob: | VSVSHEDPEVKFNWYVDGVEVHNAKTKPREEQYNSTYRVV |
| | S354C-T366W) | SVLTVLHQDWLNGKEYKCKVSNKALPAPIEKTISKAKGQPR |
| | | EPQVYTLPPCRDELTKNQVSLWCLVKGFYPSDIAVEWESNG |
| | | QPENNYKTTPPVLDSDGSFFLYSKLTVDKSRWQQGNVFSCS |
| | | VMHEALKFHYTQKSLSLSPGK |
| 409 | Fc (LALADS-KF- | DKTHTCPPCPAPEAAGGPSVFLFPPKPKDTLMISRTPEVTCVV |
| | KIH Hole: Y349C- | VSVSHEDPEVKFNWYVDGVEVHNAKTKPREEQYNSTYRVV |
| | T366S-L368A- | SVLTVLHQDWLNGKEYKCKVSNKALPAPIEKTISKAKGQPR |
| | Y407V) | EPQVCTLPPSRDELTKNQVSLSCAVKGFYPSDIAVEWESNGQ |
| | | PENNYKTTPPVLDSDGSFFLVSKLTVDKSRWQQGNVFSCSV |
| | | MHEALKFHYTQKSLSLSPGK |
| 410 | Fc (LALADS-KF- | DKTHTCPPCPAPEAAGGPSVFLFPPKPKDTLMISRTPEVTCVV |
| | KIH Knob: | VSVSHEDPEVKFNWYVDGVEVHNAKTKPREEQYNSTYRVV |
| | K409W) | SVLTVLHQDWLNGKEYKCKVSNKALPAPIEKTISKAKGQPR |
| | | EPQVYTLPPSRDELTKNQVSLTCLVKGFYPSDIAVEWESNGQ |
| | | PENNYKTTPPVLDSDGSFFLYSWLTVDKSRWQQGNVFSCSV |
| | | MHEALKFHYTQKSLSLSPGK |
| 411 | Fc (LALADS-KF- | DKTHTCPPCPAPEAAGGPSVFLFPPKPKDTLMISRTPEVTCVV |
| | KIH Hole: | VSVSHEDPEVKFNWYVDGVEVHNAKTKPREEQYNSTYRVV |
| | D399V-F405T) | SVLTVLHQDWLNGKEYKCKVSNKALPAPIEKTISKAKGQPR |
| | | EPQVYTLPPSRDELTKNQVSLTCLVKGFYPSDIAVEWESNGQ |
| | | PENNYKTTPPVLVSDGSFTLYSKLTVDKSRWQQGNVFSCSV |
| | | MHEALKFHYTQKSLSLSPGK |
| 412 | Fc (LALADS-KF- | DKTHTCPPCPAPEAAGGPSVFLFPPKPKDTLMISRTPEVTCVV |
| | KIH Knob: | VSVSHEDPEVKFNWYVDGVEVHNAKTKPREEQYNSTYRVV |
| | E357W) | SVLTVLHQDWLNGKEYKCKVSNKALPAPIEKTISKAKGQPR |
| | | EPQVYTLPPSRDWLTKNQVSLTCLVKGFYPSDIAVEWESNG |
| | | QPENNYKTTPPVLDSDGSFFLYSKLTVDKSRWQQGNVFSCS |
| | | VMHEALKFHYTQKSLSLSPGK |
| 413 | Fc (LALADS-KF- | DKTHTCPPCPAPEAAGGPSVFLFPPKPKDTLMISRTPEVTCVV |
| | KIH Hole: Y349S) | VSVSHEDPEVKFNWYVDGVEVHNAKTKPREEQYNSTYRVV |
| | | SVLTVLHQDWLNGKEYKCKVSNKALPAPIEKTISKAKGQPR |
| | | EPQVSTLPPSRDELTKNQVSLTCLVKGFYPSDIAVEWESNGQ |
| | | PENNYKTTPPVLDSDGSFFLYSKLTVDKSRWQQGNVFSCSV |
| | | MHEALKFHYTQKSLSLSPGK |

KIH-Knob and Hole. KIH substitution position numbers are according to Eu.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

What is claimed:

1. A monovalent interleukin 12 (IL-12) heterodimeric Fc protein, comprising
(a) an IL-12p35 subunit directly or indirectly linked to the N-terminus of a first immunoglobulin fragment crystallizable (Fc) region, and;
(b) an IL-12p40 subunit directly or indirectly linked to the N-terminus of a second Fc region;
wherein (i) the first Fc region and the second Fc region each comprise one or more amino acid substitutions that promote heterodimerization; (ii) at least one of the IL-12p35 and the IL-12p40 subunits of the monovalent IL-12 heterodimer Fc protein is a modified variant of the corresponding IL-12p35 or IL-12p40 subunits of wild-type IL-12, comprising an amino acid sequence having one or more amino acid substitutions, as applicable; and, (iii) the monovalent IL-12 heterodimeric Fc protein displays reduced potency compared to a monovalent IL-12 heterodimeric Fc protein comprising wild-type IL-12p35 and IL-12p40 subunits.

2. The monovalent IL-12 heterodimeric Fc protein of claim 1, wherein the potency is determined by measuring Signal Transducer and Activator of Transcription 4 (STAT4) protein induction in a cell-based assay comprising human embryo kidney (HEK) cells stably transfected with one or more nucleic acid molecules that express an IL-12 receptor and a STAT4-inducible reporter gene.

3. The monovalent IL-12 heterodimeric Fc protein of claim 1, wherein the potency is reduced at least 10-fold.

4. The monovalent IL-12 heterodimeric Fc protein of claim 1, wherein the potency is reduced at least 30-fold.

5. The monovalent IL-12 heterodimeric Fc protein of claim 1, wherein the potency is reduced from 30-fold to 200-fold.

6. The monovalent IL-12 heterodimeric Fc protein of claim 1, wherein the IL-12p35 subunit includes a deletion of the N-terminal hexapeptide RNLPVA (SEQ ID NO: 238).

7. The monovalent IL-12 heterodimeric Fc protein of claim 1, wherein the IL-12p35 subunit is a modified variant comprising an I41S, E44R, D159K, D159R, Y161S, Y161T, or Y161A amino acid substitution, wherein the amino acid positions correspond to amino acid positions I41, E44, D159, and Y161 as set forth in SEQ ID NO: 133.

8. The monovalent IL-12 heterodimeric Fc protein of claim 1, wherein the IL-12p35 subunit is a modified variant comprising an I41S, E44R, D159K, D159R, Y161S, Y161T, or Y161A amino acid substitution amino acid substitution, wherein the amino acid positions correspond to amino acid positions I41, E44, D159, and Y161 as set forth in SEQ ID NO: 133, and wherein the IL-12p35 subunit further includes a deletion of the N-terminal hexapeptide RNLPVA (SEQ ID NO: 238).

9. The monovalent IL-12 heterodimeric Fc protein of claim 1, wherein the IL-12p35 subunit is a modified variant comprising an I41S, E44R, D159K, D159R, Y161S, Y161T, or Y161A amino acid substitution amino acid substitution, wherein the amino acid positions correspond to amino acid positions I41, E44, D159, and Y161 as set forth in SEQ ID NO: 133, and the IL-12p40 subunit comprises a wild-type amino acid sequence.

10. The monovalent IL-12 heterodimeric Fc protein of claim 1, wherein the IL-12p35 subunit is a modified variant comprising an I41S, E44R, D159K, D159R, Y161S, Y161T, or Y161A amino acid substitution amino acid substitution, wherein the amino acid positions correspond to amino acid positions I41, E44, D159, and Y161 as set forth in SEQ ID NO: 133, wherein the IL-12p35 subunit further comprises a deletion of the N-terminal hexapeptide RNLPVA (SEQ ID NO: 238), and the IL-12p40 subunit comprises a wild-type amino acid sequence.

11. The monovalent IL-12 heterodimeric Fc protein of claim 1, wherein the IL12p40 subunit is a modified variant comprising a D87L, K104E, or K104D amino acid substitution, wherein the amino acid positions correspond to amino acid positions D87 and K104 as set forth in SEQ ID NO: 134.

12. The monovalent IL-12 heterodimeric Fc protein of claim 1, wherein the IL12p40 subunit is a modified variant comprising a D87L, K104E, or K104D amino acid substitution, wherein the amino acid positions correspond to amino acid positions D87 and K104 as set forth in SEQ ID NO: 134, and the IL-12p35 subunit comprises a wild-type amino acid sequence or a wild-type amino acid sequence that further includes a deletion of the N-terminal hexapeptide RNLPVA (SEQ ID NO: 238).

13. The monovalent IL-12 heterodimeric Fc protein of claim 1, wherein the IL-12p35 subunit is a modified variant comprising an I41S, E44R, D159K, D159R, Y161S, Y161T, or Y161A amino acid substitution amino acid substitution, wherein the amino acid positions correspond to amino acid positions I41, E44, D159, and Y161 as set forth in SEQ ID NO: 133, wherein the IL-12p35 subunit further includes a deletion of the N-terminal hexapeptide RNLPVA (SEQ ID NO: 238), and wherein the IL-12p40 subunit is a modified variant comprising a D87L, K104E, or K104D amino acid substitution, wherein the amino acid positions correspond to amino acid positions D87 and K104 as set forth in SEQ ID NO: 134.

14. The monovalent IL-12 heterodimeric Fc protein of claim 1, wherein the IL-12p35 subunit is a modified variant comprising an I41S, E44R, D159K, D159R, Y161S, Y161T, or Y161A amino acid substitution amino acid substitution, wherein the amino acid positions correspond to amino acid positions I41, E44, D159, and Y161 as set forth in SEQ ID NO: 133, and wherein the IL-12p40 subunit is a modified variant comprising amino acid substitutions selected from P143A/P215A, P143A/P215A/K260A/R261S/V303I, P143A/P215A/K260S/R261S/V303I, K260A/R261S/V303I, K260S/R261S/V303I, K260A/R261S, and K260S/R261S, wherein the amino acid positions correspond to amino acid positions P143, P215, K260, R261, and V303 as set forth in SEQ ID NO: 134.

15. The monovalent IL-12 heterodimeric Fc protein of claim 1, wherein the IL-12p35 subunit is a modified variant comprising a Y161S or Y161T amino acid substitution, wherein the amino acid position corresponds to amino acid position Y161 as set forth in SEQ ID NO: 133, and the IL-12p40 subunit comprises a wild-type amino acid sequence or is a modified variant comprising a D87L, K104E, or K104D amino acid substitution, wherein the amino acid positions correspond to amino acid positions D87 and K104 as set forth in SEQ ID NO: 134.

16. The monovalent IL-12 heterodimeric Fc protein of claim 1, wherein the IL-12 p35 subunit is a modified variant comprising an I41S, E44R, D159K, D159R, Y161S, Y161T, or Y161A amino acid substitution amino acid substitution, wherein the amino acid positions correspond to amino acid positions an I41, E44, D159, or Y161 amino acid substitution as set forth in SEQ ID NO: 133, wherein the IL-12p35 subunit further includes a deletion of the N-terminal hexapeptide RNLPVA (SEQ ID NO: 238), and wherein the p40 subunit is a modified variant comprising amino acid substitutions selected from P143A/P215A, P143A/P215A/K260A/R261S/V303I, P143A/P215A/K260S/R261S/V303I, K260A/R261S/V303I, K260S/R261S/V303I, K260A/R261S, and K260S/R261S, wherein the amino acid positions correspond to amino acid positions P143, P215, K260, R261, and V303 as set forth in SEQ ID NO: 134.

17. The monovalent IL-12 heterodimeric Fc protein of claim 1, wherein the IL-12p35 subunit is a modified variant comprising a Y161S or Y161T amino acid substitution, wherein the amino acid position corresponds to amino acid position Y161 as set forth in SEQ ID NO: 133, wherein the IL-12p35 subunit further includes a deletion of the N-terminal hexapeptide RNLPVA (SEQ ID NO: 238), and the IL-12p40 subunit comprises a wild-type amino acid sequence or is a modified variant comprising a D87L, K104E, or K104D amino acid substitution, wherein the amino acid positions correspond to amino acid positions D87 and K104 as set forth in SEQ ID NO: 134.

18. The monovalent IL-12 heterodimeric Fc protein of claim 1, wherein the IL-12p35 subunit is a modified variant comprising the amino acid sequence of SEQ ID NO: 140, 147, 148, 152, 153, or 154, and the IL12p40 subunit is a wild-type IL-12p40 subunit comprising the amino acid sequence set forth in SEQ ID NO: 134 or the IL-12p40 subunit is a modified variant comprising the amino acid sequence set forth in SEQ ID NO: 161, 166, 167, 192, 193, 194, 195, 196, 197, or 198.

19. The monovalent IL-12 heterodimeric Fc protein of claim 1, wherein
(i) the first Fc region comprises the amino acid substitutions S354C and T366W and the second Fc region comprises the amino acid substitutions Y349C, T366S, L368A, and Y407V, wherein the amino acid positions are according to the EU Index;
(ii) the first Fc region comprises the amino acid substitutions Y349C, T366S, L368A, and Y407V and the second Fc region comprises the amino acid substitutions S354C and T366W, wherein the amino acid positions are according to the EU Index;
(iii) the first Fc region comprises the amino acid substitution K409W and the second Fc region comprises the amino acid substitutions D399V and F405T, wherein the amino acid positions are according to the EU Index;
(iv) the first Fc region comprises the amino acid substitutions D399V and F405T and the second Fc region comprises the amino acid substitution K409W, wherein the amino acid positions are according to the EU Index;
(v) the first Fc region comprises the amino acid substitution K357W and the second Fc region comprises the amino acid substitution Y349S, wherein the amino acid positions are according to the EU Index; or,
(vi) the first Fc region comprises the amino acid substitution Y349S and the second Fc region comprises the amino acid substitution K357W, wherein the amino acid positions are according to the EU Index.

20. The monovalent IL-12 heterodimeric Fc protein of claim 1, wherein
(a)(i) the IL-12p35 subunit is a modified variant comprising an I41S, E44R, D159K, D159R, Y161S, Y161T, or Y161A amino acid substitution directly or indirectly linked to the N-terminus of a first Fc region comprising the amino acid substitutions S354C and T366W, wherein the Fc amino acid positions are according to the EU Index and the amino acid positions of the IL-12p35 subunit variant correspond to amino acid positions I41, E44, D159, or Y161 as set forth in SEQ ID NO: 133; and,
(a)(ii) the IL-12p40 subunit is directly or indirectly linked to the N-terminus of a second Fc region comprising the amino acid substitutions Y349C, T366S, L368A, and Y407V, wherein the amino acid positions are according to the EU Index and the IL-12p40 subunit is wild-type; or
(b)(i) the IL-12 p40 subunit is directly or indirectly linked to the N-terminus of a first Fc region comprising the amino acid substitutions S354C and T366W, wherein the amino acid positions are according to the EU Index and the IL-12p40 subunit is wild-type; and
(b)(ii) the IL-12p35 subunit is a modified variant comprising an I41S, E44R, D159K, D159R, Y161S, Y161T, or Y161A amino acid substitution amino acid substitution directly or indirectly linked to the N-terminus of a second Fc region comprising the amino acid substitutions Y349C, T366S, L368A, and Y407V wherein the Fc amino acid positions are according to the EU Index and the amino acid positions of the IL-12p35 subunit variant correspond to amino acid positions I41, E44, D159, or Y161 as set forth in SEQ ID NO: 133.

21. The monovalent IL-12 heterodimeric Fc protein of claim 1, wherein the first Fc region and the second Fc region each comprise one or more amino acid substitutions that increase serum half-life of the monovalent IL-12 heterodimeric Fc protein compared to the half-life of a monovalent IL-12 heterodimeric Fc protein comprising a wild-type Fc.

22. The monovalent IL-12 heterodimeric Fc protein of claim 21, wherein the first Fc region and the second Fc region each comprise amino acid substitutions selected from the group consisting of (i) M252Y, S254T, and T256E; (ii) M428L and N434S; (iii) L309D, Q311H, and N434S; and (iv) H433K and N434F, wherein the amino acid positions are according to the Eu numbering scheme.

23. The monovalent IL-12 heterodimeric Fc protein of claim 1, wherein the first Fc region and the second Fc region each comprise one or more amino acid substitutions that reduce an Fc region effector function.

24. The monovalent IL-12 heterodimeric Fc protein of claim 23, wherein the first Fc region and the second Fc region each comprise amino acid substitutions selected from the group consisting of (i) L234A, L235A, and D265S; (ii) L234A, L235A, and P329G; (iii) L234A and L235A; (iv) H268Q, V309L, A330S, and P331S; (v) V234A, G237A, P338S, H268A, and V309L; and (vi) N297X wherein X is A, Q, or G; wherein the amino acid positions are according to the Eu numbering scheme.

25. The monovalent IL-12 heterodimeric Fc protein of claim 1, wherein the IL-12p35 subunit or modified variant thereof is indirectly linked to the N-terminus of the first Fc region via a first peptide linker or non-peptidyl polymer and the wherein the IL-12p40 subunit or modified variant thereof is indirectly linked to the N-terminus of the second Fc region via a second peptide linker or non-peptidyl polymer.

26. The monovalent IL-12 heterodimeric Fc protein of claim 25, wherein the first peptide linker comprises the amino acid sequence GGGGSGGGGSGGGGS (SEQ ID NO: 206) and the second peptide linker comprises the amino acid sequence GGGG (SEQ ID NO: 202).

27. The monovalent IL-12 heterodimeric Fc protein of claim 25, wherein the first and second non-peptidyl polymer are independently selected from polyethylene glycol, polypropylene glycol, copolymers of ethylene glycol and propylene glycol, polyoxyethylated polyols, polyvinyl alcohol, polysaccharides, polyvinyl ethyl ether, polylactic acid, polylacticglycolic acid, lipid polymers, hyaluronic acid, and combinations thereof.

28. The monovalent IL-12 heterodimeric Fc protein of claim 1, wherein the monovalent IL-12 heterodimeric Fc protein comprises:
(a) an IL-12p35 polypeptide comprising an amino acid substitution selected from the group consisting of SEQ ID NO: 133, SEQ ID NO: 140, SEQ ID NO: 148, SEQ ID NO: 149, SEQ ID NO: 152, SEQ ID NO: 153, SEQ ID NO: 154, SEQ ID NO: 239, SEQ ID NO: 266, SEQ ID NO: 274, SEQ ID NO: 275, SEQ ID NO: 278, SEQ ID NO: 279, and SEQ ID NO: 280 linked to an Fc protein comprising an amino acid sequence selected from the group consisting of SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, and SEQ ID NO: 412; and
(b) an IL-12p40 polypeptide comprising a wild-type amino acid sequence or an amino acid substitution selected from the group consisting of SEQ ID NO: 134, SEQ ID NO: 161, SEQ ID NO:166, and SEQ ID NO: 167 linked to an Fc protein comprising an amino acid sequence selected from the group consisting of SEQ ID NO: 209, SEQ ID NO: 211, SEQ ID NO: 213, SEQ ID NO: 215, SEQ ID NO: SEQ ID NO: 217, SEQ ID NO: 219, SEQ ID NO: 221, SEQ ID NO:397, SEQ ID NO: 399, SEQ ID NO: 401, SEQ ID NO: 403, SEQ ID NO: 405, SEQ ID NO: 407, SEQ ID NO: 409, SEQ ID NO: 411, and SEQ ID NO: 413.

29. The monovalent IL-12 heterodimeric Fc protein of claim 1, wherein the monovalent IL-12 heterodimeric Fc protein comprises:
(a) an IL-12p35 polypeptide comprising an amino acid substitution selected from the group consisting of SEQ ID NO: 133, SEQ ID NO: 140, SEQ ID NO: 148, SEQ ID NO: 149, SEQ ID NO: 152, SEQ ID NO: 153, SEQ ID NO: 154, SEQ ID NO: 239, SEQ ID NO: 266, SEQ ID NO: 274, SEQ ID NO: 275, SEQ ID NO: 278, SEQ ID NO: 279, and SEQ ID NO: 280 linked to an Fc protein comprising an amino acid sequence selected from the group consisting of SEQ ID NO: 209, SEQ ID NO: 211, SEQ ID NO: 213, SEQ ID NO: 215, SEQ ID NO: SEQ ID NO: 217, SEQ ID NO: 219, SEQ ID NO: 221, SEQ ID NO:397, SEQ ID NO: 399, SEQ ID NO: 401, SEQ ID NO: 403, SEQ ID NO: 405, SEQ ID NO: 407, SEQ ID NO: 409, SEQ ID NO: 411, and SEQ ID NO: 413; and
(b) an IL-12p40 polypeptide comprising a wild-type amino acid sequence or an amino acid substitution selected from the group consisting of SEQ ID NO: 134, SEQ ID NO: 161, SEQ ID NO:166, and SEQ ID NO: 167 linked to an Fc protein comprising an amino acid sequence selected from the group consisting of SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, and SEQ ID NO: 412.

30. The monovalent IL-12 heterodimeric Fc protein of claim 1, wherein the monovalent IL-12 heterodimeric Fc protein comprises:
(a) a first polypeptide comprising the amino acid sequence set forth in SEQ ID NO: 8 and a second polypeptide comprising the amino acid sequence set forth in SEQ ID NO: 2;
(b) a first polypeptide comprising the amino acid sequence set forth in SEQ ID NO: 15 and a second polypeptide comprising the amino acid sequence set forth in SEQ ID NO: 2;
(c) a first polypeptide comprising the amino acid sequence set forth in SEQ ID NO: 16 and a second polypeptide comprising the amino acid sequence set forth in SEQ ID NO: 2;
(d) a first polypeptide comprising the amino acid sequence set forth in SEQ ID NO: 17 and a second polypeptide comprising the amino acid sequence set forth in SEQ ID NO: 2;
(e) a first polypeptide comprising the amino acid sequence set forth in SEQ ID NO: 20 and a second polypeptide comprising the amino acid sequence set forth in SEQ ID NO: 2;
(f) a first polypeptide comprising the amino acid sequence set forth in SEQ ID NO: 21 and a second polypeptide comprising the amino acid sequence set forth in SEQ ID NO: 2; or
(g) a first polypeptide comprising the amino acid sequence set forth in SEQ ID NO: 22 and a second polypeptide comprising the amino acid sequence set forth in SEQ ID NO: 2.

31. The monovalent IL-12 heterodimeric Fc protein of claim 1, wherein the monovalent IL-12 heterodimeric Fc protein comprises:
(a) a first polypeptide comprising the amino acid sequence set forth in SEQ ID NO: 74 and a second polypeptide comprising the amino acid sequence set forth in SEQ ID NO: 68;
(b) a first polypeptide comprising the amino acid sequence set forth in SEQ ID NO: 81 and a second polypeptide comprising the amino acid sequence set forth in SEQ ID NO: 68;
(c) a first polypeptide comprising the amino acid sequence set forth in SEQ ID NO: 82 and a second polypeptide comprising the amino acid sequence set forth in SEQ ID NO: 68;
(d) a first polypeptide comprising the amino acid sequence set forth in SEQ ID NO: 83 and a second polypeptide comprising the amino acid sequence set forth in SEQ ID NO: 68;
(e) a first polypeptide comprising the amino acid sequence set forth in SEQ ID NO: 86 and a second polypeptide comprising the amino acid sequence set forth in SEQ ID NO: 68;
(f) a first polypeptide comprising the amino acid sequence set forth in SEQ ID NO: 87 and a second polypeptide comprising the amino acid sequence set forth in SEQ ID NO: 68; or
(g) a first polypeptide comprising the amino acid sequence set forth in SEQ ID NO: 88 and a second polypeptide comprising the amino acid sequence set forth in SEQ ID NO: 68.

32. A pharmaceutical composition comprising the monovalent IL-12 heterodimeric Fc protein of claim 1 and a pharmaceutical carrier or diluent.

33. A method for treating a cancer comprising:
administering a therapeutically effective amount of the monovalent IL-12 heterodimeric Fc protein of claim 1 or a pharmaceutical composition thereof to an individual in need thereof.

34. A therapy for treating cancer wherein a monovalent IL-12 heterodimeric Fc protein of claim 1 or the pharmaceutical composition thereof is provided in combination with an anti-cancer treatment selected from radiation treatment, chemotherapy treatment, immune checkpoint inhibitor treatment, or antibody-drug conjugate treatment.

35. A method for treating a cancer comprising:
administering a therapeutically effective amount of the monovalent IL-12 heterodimeric Fc protein of claim 1 or a pharmaceutical composition thereof and an anti-cancer treatment selected from radiation treatment, chemotherapy treatment, immune checkpoint inhibitor treatment, or antibody-drug conjugate treatment to an individual in need thereof.

36. A method for producing a monovalent IL-12 heterodimeric Fc protein, comprising:
(a) providing a first nucleic acid molecule encoding a first polypeptide comprising an IL-12p35 subunit fused to the N-terminus of a first immunoglobulin fragment crystallizable (Fc) region and a second nucleic acid molecule encoding a second polypeptide comprising an IL-12p40 subunit fused to the N-terminus of a second Fc region, wherein the first Fc region and the second Fc region each comprise one or more amino acid substitutions that promote heterodimerization, and wherein the IL-12p35 subunit of the monovalent IL-12 heterodimer Fc protein comprises one or more amino acid substitutions that reduce the potency of the monovalent IL-12 heterodimeric Fc protein compared to a monovalent IL-12 heterodimeric Fc protein comprising wild-type IL-12p35 and IL-12p40 subunits;
(b) transfecting a host cell with the first nucleic acid molecule and the second nucleic acid molecule to produce a transfected host cell;
(c) cultivating the host cell in a culture medium under conditions for the production of the first polypeptide and the second polypeptide, which are excreted into the culture medium and associated into a monovalent IL-12 heterodimeric Fc protein comprising a first polypeptide and a second polypeptide; and
(d) recovering the monovalent IL-12 heterodimeric Fc proteins from the culture medium.

37. The method of claim 36, wherein the first polypeptide comprises the amino acid sequence set forth in SEQ ID NO: 74, 81, 82, 83, 86, 87, or 88 and the second polypeptide comprises the amino acid sequence set forth in SEQ ID NO: 68; or the first polypeptide comprises the amino acid sequence set forth in SEQ ID NO: 8, 15, 16, 17, 20, 21, or 22 and the second polypeptide comprises the amino acid sequence set forth in SEQ ID NO: 2.

38. The method of claim 36, wherein the first nucleic acid molecule comprises the nucleotide sequence set forth in SEQ ID NO: 223, 224, 25, 226, 227, 228, or 229 and the second nucleic acid molecule comprises the nucleotide sequence set forth in SEQ ID NO: 230 or the first nucleic acid molecule comprises the nucleotide sequence set forth in SEQ ID NO: 234, 235, 236, 240, 241, 242, or 243 and the second nucleic acid molecule comprises the nucleotide sequence set forth in SEQ ID NO: 237.

39. A polypeptide comprising the amino acid sequence set forth in SEQ ID NO: 1, 2, 3, 4, 5, 6, 7, 8, 9, 10, 11, 12 13, 14 15, 16 17, 18, 19, 20, 21, 22, 23, 24 25 26, 27, 28, 29, 30, 31, 32, 33, 34, 35 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, or 66; or the amino acid sequence set forth in SEQ ID NO: 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127,128, 129, 130, 131, or 132; or the amino acid sequence set forth in SEQ ID NO: 133, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, or 198; or the amino acid set forth in SEQ ID NO: 261, 262, 263, 264, 265, 266, 267, 268, 269, 270, 271, 272, 273, 274, 275, 276, 277, 278, 278, 279, 280, 281, 282, or 283; or the amino acid set forth in SEQ ID NO: 284, 285, 286, 287, 288, 289, 290, 291, 292, 293, 294, 295, 296, 297, 298, 299, 300, 301, 302, or 303; or the amino acid set forth in SEQ ID NO: 304, 305, 306, 307, 308, 309, 310, 311, 312, 313, 314, 315, 316, 317, 318, 319, 320, 321, 322, 323, 324, 325, or 326; or the amino acid set forth in SEQ ID NO: 327, 328, 329, 330, 331, 332, 333, 334, 335,336, 337, 338, 339, 340, 341, 342, 343, 344, 345, or 346; or the amino acid set forth in SEQ ID NO: 347, 348, 349, 350, 351, 352, 353, 354, 356, 357, 358, 359, 360, 361, 362, 363, 364, 365, 366, 367, 368, or 369; or the amino acid set forth in SEQ ID NO: 370, 371, 372, 373, 374, 375, 376, 377, 378, 379, 380, 381, 382, 383, 384, 385, 386, 387, 388, or 389.

40. A nucleic acid molecule comprising the nucleotide sequence set forth in SEQ ID NO: 223, 224, 225, 226, 227, 228, 229, 230, 231, 232, 233, 249, or 250; or the nucleotide sequence set forth in SEQ ID NO: 234, 235, 236, 237, 240, 241, 242, 243, 244, 245, 246, 247, or 248; or the nucleotide sequence set forth in SEQ ID NO: 251, 252, 253, 254, 255, 256, 257, 258, 259, or 260.

41. A composition comprising a monovalent IL-12 heterodimeric Fc protein of claim 1, wherein 14.5% to 25% of the N-glycans in the composition are sialylated; or wherein 15% of the N-glycans in the composition are sialylated; or wherein 20% of the N-glycans in the composition are sialylated; or wherein 25% of the N-glycans in the composition are sialylated.


SEQUENCE LISTING (413 sequences)

SEQ ID NO: 1 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3 - protein, 433 aa, synthetic construct
TPDPGMFPCLHHSQNLLRAVSNMLQKARQTLEFYPCTSEKIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 433 aa, synthetic construct
TPDPGMFPCLHHSQNLLRAVSNMLQKARQTLEFYPCTSERIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 433 aa, synthetic construct
TPDPGMFPCLHHSQNLLRAVSNMLQKARQTLEFYPCTSEWIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 433 aa, synthetic construct
TPDPGMFPCLHHSQNLLRAVSNMLQKARQTLEFYPCTSEEADHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 433 aa, synthetic construct
TPDPGMFPCLHHSQNLLRAVSNMLQKARQTLEFYPCTSEEVDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 433 aa, synthetic construct
TPDPGMFPCLHHSQNLLRAVSNMLQKARQTLEFYPCTSEESDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 433 aa, synthetic construct
TPDPGMFPCLHHSQNLLRAVSNMLQKARQTLEFYPCTSEETDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 433 aa, synthetic construct
TPDPGMFPCLHHSQNLLRAVSNMLQKARQTLEFYPCTSEEIAHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 433 aa, synthetic construct
TPDPGMFPCLHHSQNLLRAVSNMLQKARQTLEFYPCTSEEILHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 433 aa, synthetic construct
TPDPGMFPCLHHSQNLLRAVSNMLQKARQTLEFYPCTSEEIWHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 433 aa, synthetic construct
TPDPGMFPCLHHSQNLLRAVSNMLQKARQTLEFYPCTSEEIDHADITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 433 aa, synthetic construct
TPDPGMFPCLHHSQNLLRAVSNMLQKARQTLEFYPCTSEEIDHKDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 433 aa, synthetic construct
TPDPGMFPCLHHSQNLLRAVSNMLQKARQTLEFYPCTSEEIDHRDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPRFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPYFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPFFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFSKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFAKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFWKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYETKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYDTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKEEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 27 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKYEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 28 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEAGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 29 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKELGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 30 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGAWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 31 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGSWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 32 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGDWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 33 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGPWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 34 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNETFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 35 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNDTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNWTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 37 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNSTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 38 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 433 aa, synthetic construct
TPDPGMFPCLHHSQNLLRAVSNMLQKARQTLEGYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 433 aa, synthetic construct
TPDPGMFPCLHHSQNLLRAVSNMLQKARQTLEKYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 433 aa, synthetic construct
TPDPGMFPCLHHSQNLLRAVSNMLQKARQTLESYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 433 aa, synthetic construct
TPDPGMFPCLHHSQNLLRAVSNMLQKARQTLEFGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 433 aa, synthetic construct
TPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 433 aa, synthetic construct
TPDPGMFPCLHHSQNLLRAVSNMLQKARQTLEFTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 433 aa, synthetic construct
TPDPGMFPCLHHSQNLLRAVSNMLQKARQTLEFYQCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 433 aa, synthetic construct
TPDPGMFPCLHHSQNLLRAVSNMLQKARQTLEFYECTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 433 aa, synthetic construct
TPDPGMFPCLHHSQNLLRAVSNMLQKARQTLESTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 433 aa, synthetic construct
TPDPGMFPCLHHSQNLLRAVSNMLQKARQTLESSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 433 aa, synthetic construct
TPDPGMFPCLHHSQNLLRAVSNMLQKARQTLEGYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 433 aa, synthetic construct
TPDPGMFPCLHHSQNLLRAVSNMLQKARQTLEKYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 433 aa, synthetic construct
TPDPGMFPCLHHSQNLLRAVSNMLQKARQTLESYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 433 aa, synthetic construct
TPDPGMFPCLHHSQNLLRAVSNMLQKARQTLEFGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 433 aa, synthetic construct
TPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 433 aa, synthetic construct
TPDPGMFPCLHHSQNLLRAVSNMLQKARQTLEFTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 434 aa, synthetic construct
TPDPGMFPCLHHSQNLLRAVSNMLQKARQTLESTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 433 aa, synthetic construct
TPDPGMFPCLHHSQNLLRAVSNMLQKARQTLESSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 433 aa, synthetic construct
TPDPGMFPCLHHSQNLLRAVSNMLQKARQTLESGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKELGIWSTDILKDQKEPKNETFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 59 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKELGIWSTDILKDQKEPKNDTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 60 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDAQGVTCGAATLSAERVRGDNKEYEYSVECQEDSACPAAEESLPIEVMVDAVHKLKYENYTSSFFIRDIIKPDAPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 61 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDAQGVTCGAATLSAERVRGDNKEYEYSVECQEDSACPAAEESLPIEVMVDAVHKLKYENYTSSFFIRDIIKPDAPKNLQLKPLKNSRQVEVSWEYPDTWSTPHSYFSLTFCVQVQGKSASEKKDRVFTDKTSATVICRKNASISVRAQDRYYSSSWSEWASIPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 62 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDAQGVTCGAATLSAERVRGDNKEYEYSVECQEDSACPAAEESLPIEVMVDAVHKLKYENYTSSFFIRDIIKPDAPKNLQLKPLKNSRQVEVSWEYPDTWSTPHSYFSLTFCVQVQGKSSSEKKDRVFTDKTSATVICRKNASISVRAQDRYYSSSWSEWASIPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 63 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSASEKKDRVFTDKTSATVICRKNASISVRAQDRYYSSSWSEWASIPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 64 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSSSEKKDRVFTDKTSATVICRKNASISVRAQDRYYSSSWSEWASIPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 65 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSASEKKDRVFTDKTSATVICRKNASISVRAQDRYYSSSWSEWASVPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 66 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSSSEKKDRVFTDKTSATVICRKNASISVRAQDRYYSSSWSEWASVPCSGGGGDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 67 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 69 - protein, 433 aa, synthetic construct
TPDPGMFPCLHHSQNLLRAVSNMLQKARQTLEFYPCTSEKIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 433 aa, synthetic construct
TPDPGMFPCLHHSQNLLRAVSNMLQKARQTLEFYPCTSERIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 433 aa, synthetic construct
TPDPGMFPCLHHSQNLLRAVSNMLQKARQTLEFYPCTSEWIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 433 aa, synthetic construct
TPDPGMFPCLHHSQNLLRAVSNMLQKARQTLEFYPCTSEEADHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 433 aa, synthetic construct
TPDPGMFPCLHHSQNLLRAVSNMLQKARQTLEFYPCTSEEVDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 433 aa, synthetic construct
TPDPGMFPCLHHSQNLLRAVSNMLQKARQTLEFYPCTSEESDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 433 aa, synthetic construct
TPDPGMFPCLHHSQNLLRAVSNMLQKARQTLEFYPCTSEETDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 433 aa, synthetic construct
TPDPGMFPCLHHSQNLLRAVSNMLQKARQTLEFYPCTSEEIAHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 433 aa, synthetic construct
TPDPGMFPCLHHSQNLLRAVSNMLQKARQTLEFYPCTSEEILHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 433 aa, synthetic construct
TPDPGMFPCLHHSQNLLRAVSNMLQKARQTLEFYPCTSEEIWHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 433 aa, synthetic construct
TPDPGMFPCLHHSQNLLRAVSNMLQKARQTLEFYPCTSEEIDHADITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 433 aa, synthetic construct
TPDPGMFPCLHHSQNLLRAVSNMLQKARQTLEFYPCTSEEIDHKDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 433 aa, synthetic construct
TPDPGMFPCLHHSQNLLRAVSNMLQKARQTLEFYPCTSEEIDHRDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPRFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPYFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPFFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFSKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFAKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFWKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYETKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYDTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKEEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 93 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKYEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 94 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEAGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 95 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKELGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 96 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGAWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 97 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGSWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 98 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGDWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 99 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGPWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 100 - protein, 537 aa, synthetic construct; identical to SEQ ID NO: 34

SEQ ID NO: 101 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNDTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 102 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNWTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 103 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNSTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 104 - protein, 433 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 433 aa, synthetic construct
TPDPGMFPCLHHSQNLLRAVSNMLQKARQTLEGYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 433 aa, synthetic construct
TPDPGMFPCLHHSQNLLRAVSNMLQKARQTLEKYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 433 aa, synthetic construct; identical to SEQ ID NO: 41

SEQ ID NO: 108 - protein, 433 aa, synthetic construct
TPDPGMFPCLHHSQNLLRAVSNMLQKARQTLEFGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 433 aa, synthetic construct
TPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 433 aa, synthetic construct
TPDPGMFPCLHHSQNLLRAVSNMLQKARQTLEFTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 433 aa, synthetic construct
TPDPGMFPCLHHSQNLLRAVSNMLQKARQTLEFYQCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 433 aa, synthetic construct
TPDPGMFPCLHHSQNLLRAVSNMLQKARQTLEFYECTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 433 aa, synthetic construct
TPDPGMFPCLHHSQNLLRAVSNMLQKARQTLESTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 433 aa, synthetic construct
TPDPGMFPCLHHSQNLLRAVSNMLQKARQTLESSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 433 aa, synthetic construct
TPDPGMFPCLHHSQNLLRAVSNMLQKARQTLEGYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 433 aa, synthetic construct
TPDPGMFPCLHHSQNLLRAVSNMLQKARQTLEKYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 433 aa, synthetic construct
TPDPGMFPCLHHSQNLLRAVSNMLQKARQTLESYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 433 aa, synthetic construct
TPDPGMFPCLHHSQNLLRAVSNMLQKARQTLEFGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 433 aa, synthetic construct
TPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 433 aa, synthetic construct
TPDPGMFPCLHHSQNLLRAVSNMLQKARQTLEFTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 433 aa, synthetic construct
TPDPGMFPCLHHSQNLLRAVSNMLQKARQTLESTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 433 aa, synthetic construct
TPDPGMFPCLHHSQNLLRAVSNMLQKARQTLESSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 433 aa, synthetic construct
TPDPGMFPCLHHSQNLLRAVSNMLQKARQTLESGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKELGIWSTDILKDQKEPKNETFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 125 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKELGIWSTDILKDQKEPKNDTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 126 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDAQGVTCGAATLSAERVRGDNKEYEYSVECQEDSACPAAEESLPIEVMVDAVHKLKYENYTSSFFIRDIIKPDAPKNLQLKPLKNSRQVEVSWEYPDTWSTPHSYFSLTFCVQVQGKSKREKKDRVFTDKTSATVICRKNASISVRAQDRYYSSSWSEWASVPCSGGGGDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 127 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDAQGVTCGAATLSAERVRGDNKEYEYSVECQEDSACPAAEESLPIEVMVDAVHKLKYENYTSSFFIRDIIKPDAPKNLQLKPLKNSRQVEVSWEYPDTWSTPHSYFSLTFCVQVQGKSASEKKDRVFTDKTSATVICRKNASISVRAQDRYYSSSWSEWASIPCSGGGGDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 128 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDAQGVTCGAATLSAERVRGDNKEYEYSVECQEDSACPAAEESLPIEVMVDAVHKLKYENYTSSFFIRDIIKPDAPKNLQLKPLKNSRQVEVSWEYPDTWSTPHSYFSLTFCVQVQGKSSSEKKDRVFTDKTSATVICRKNASISVRAQDRYYSSSWSEWASIPCSGGGGDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 129 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSASEKKDRVFTDKTSATVICRKNASISVRAQDRYYSSSWSEWASIPCSGGGGDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 130 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSSSEKKDRVFTDKTSATVICRKNASISVRAQDRYYSSSWSEWASIPCSGGGGDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 131 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSASEKKDRVFTDKTSATVICRKNASISVRAQDRYYSSSWSEWASVPCSGGGGDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 132 - protein, 537 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSSSEKKDRVFTDKTSATVICRKNASISVRAQDRYYSSSWSEWASVPCSGGGGDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 133 - protein, 191 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 134 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 135 - protein, 191 aa, synthetic construct
TPDPGMFPCLHHSQNLLRAVSNMLQKARQTLEFYPCTSEKIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 136 - protein, 191 aa, synthetic construct
TPDPGMFPCLHHSQNLLRAVSNMLQKARQTLEFYPCTSERIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 137 - protein, 191 aa, synthetic construct
TPDPGMFPCLHHSQNLLRAVSNMLQKARQTLEFYPCTSEWIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 138 - protein, 191 aa, synthetic construct
TPDPGMFPCLHHSQNLLRAVSNMLQKARQTLEFYPCTSEEADHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 139 - protein, 191 aa, synthetic construct
TPDPGMFPCLHHSQNLLRAVSNMLQKARQTLEFYPCTSEEVDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 140 - protein, 191 aa, synthetic construct
TPDPGMFPCLHHSQNLLRAVSNMLQKARQTLEFYPCTSEESDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 141 - protein, 191 aa, synthetic construct
TPDPGMFPCLHHSQNLLRAVSNMLQKARQTLEFYPCTSEETDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 142 - protein, 191 aa, synthetic construct
TPDPGMFPCLHHSQNLLRAVSNMLQKARQTLEFYPCTSEEIAHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 143 - protein, 191 aa, synthetic construct
TPDPGMFPCLHHSQNLLRAVSNMLQKARQTLEFYPCTSEEILHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 144 - protein, 191 aa, synthetic construct
TPDPGMFPCLHHSQNLLRAVSNMLQKARQTLEFYPCTSEEIWHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 145 - protein, 191 aa, synthetic construct
TPDPGMFPCLHHSQNLLRAVSNMLQKARQTLEFYPCTSEEIDHADITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 146 - protein, 191 aa, synthetic construct
TPDPGMFPCLHHSQNLLRAVSNMLQKARQTLEFYPCTSEEIDHKDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 147 - protein, 191 aa, synthetic construct
TPDPGMFPCLHHSQNLLRAVSNMLQKARQTLEFYPCTSEEIDHRDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 148 - protein, 191 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFYKTKIKLCILLHAFRIRAVTIDRVMSYLNAS

SEQ ID NO: 149 - protein, 191 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPRFYKTKIKLCILLHAFRIRAVTIDRVMSYLNAS

SEQ ID NO: 150 - protein, 191 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPYFYKTKIKLCILLHAFRIRAVTIDRVMSYLNAS

SEQ ID NO: 151 - protein, 191 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPFFYKTKIKLCILLHAFRIRAVTIDRVMSYLNAS

SEQ ID NO: 152 - protein, 191 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFSKTKIKLCILLHAFRIRAVTIDRVMSYLNAS

SEQ ID NO: 153 - protein, 191 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNAS

SEQ ID NO: 154 - protein, 191 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFAKTKIKLCILLHAFRIRAVTIDRVMSYLNAS

SEQ ID NO: 155 - protein, 191 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFWKTKIKLCILLHAFRIRAVTIDRVMSYLNAS

SEQ ID NO: 156 - protein, 191 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYETKIKLCILLHAFRIRAVTIDRVMSYLNAS

SEQ ID NO: 157 - protein, 191 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYDTKIKLCILLHAFRIRAVTIDRVMSYLNAS

SEQ ID NO: 158 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKEEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 159 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKYEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 160 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEAGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 161 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKELGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 162 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGAWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 163 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGSWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 164 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGDWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 165 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGPWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 166 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNETFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 167 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNDTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 168 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNWTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 169 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNSTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 170 - protein, 191 aa, synthetic construct
TPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNAS

SEQ ID NO: 171 - protein, 191 aa, synthetic construct
TPDPGMFPCLHHSQNLLRAVSNMLQKARQTLEGYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNAS

SEQ ID NO: 172 - protein, 191 aa, synthetic construct
TPDPGMFPCLHHSQNLLRAVSNMLQKARQTLEKYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNAS

SEQ ID NO: 173 - protein, 191 aa, synthetic construct
TPDPGMFPCLHHSQNLLRAVSNMLQKARQTLESYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNAS

SEQ ID NO: 174 - protein, 191 aa, synthetic construct
TPDPGMFPCLHHSQNLLRAVSNMLQKARQTLEFGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNAS

SEQ ID NO: 175 - protein, 191 aa, synthetic construct
TPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNAS

SEQ ID NO: 176 - protein, 191 aa, synthetic construct
TPDPGMFPCLHHSQNLLRAVSNMLQKARQTLEFTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNAS

SEQ ID NO: 177 - protein, 191 aa, synthetic construct
TPDPGMFPCLHHSQNLLRAVSNMLQKARQTLEFYQCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNAS

SEQ ID NO: 178 - protein, 191 aa, synthetic construct
TPDPGMFPCLHHSQNLLRAVSNMLQKARQTLEFYECTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNAS

SEQ ID NO: 179 - protein, 191 aa, synthetic construct
TPDPGMFPCLHHSQNLLRAVSNMLQKARQTLESTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNAS

SEQ ID NO: 180 - protein, 191 aa, synthetic construct
TPDPGMFPCLHHSQNLLRAVSNMLQKARQTLESSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNAS

SEQ ID NO: 181 - protein, 191 aa, synthetic construct
TPDPGMFPCLHHSQNLLRAVSNMLQKARQTLEGYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNAS

SEQ ID NO: 182 - protein, 191 aa, synthetic construct
TPDPGMFPCLHHSQNLLRAVSNMLQKARQTLEKYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNAS

SEQ ID NO: 183 - protein, 191 aa, synthetic construct
TPDPGMFPCLHHSQNLLRAVSNMLQKARQTLESYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNAS

SEQ ID NO: 184 - protein, 191 aa, synthetic construct
TPDPGMFPCLHHSQNLLRAVSNMLQKARQTLEFGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNAS

SEQ ID NO: 185 - protein, 191 aa, synthetic construct
TPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNAS

SEQ ID NO: 186 - protein, 191 aa, synthetic construct
TPDPGMFPCLHHSQNLLRAVSNMLQKARQTLEFTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNAS

SEQ ID NO: 187 - protein, 192 aa, synthetic construct
TPDPGMFPCLHHSQNLLRAVSNMLQKARQTLESTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASG

SEQ ID NO: 188 - protein, 191 aa, synthetic construct
TPDPGMFPCLHHSQNLLRAVSNMLQKARQTLESSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNAS

SEQ ID NO: 189 - protein, 191 aa, synthetic construct
TPDPGMFPCLHHSQNLLRAVSNMLQKARQTLESGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNAS

SEQ ID NO: 190 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKELGIWSTDILKDQKEPKNETFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 191 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKELGIWSTDILKDQKEPKNDTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 192 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDAQGVTCGAATLSAERVRGDNKEYEYSVECQEDSACPAAEESLPIEVMVDAVHKLKYENYTSSFFIRDIIKPDAPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 193 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDAQGVTCGAATLSAERVRGDNKEYEYSVECQEDSACPAAEESLPIEVMVDAVHKLKYENYTSSFFIRDIIKPDAPKNLQLKPLKNSRQVEVSWEYPDTWSTPHSYFSLTFCVQVQGKSASEKKDRVFTDKTSATVICRKNASISVRAQDRYYSSSWSEWASIPCS

SEQ ID NO: 194 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDAQGVTCGAATLSAERVRGDNKEYEYSVECQEDSACPAAEESLPIEVMVDAVHKLKYENYTSSFFIRDIIKPDAPKNLQLKPLKNSRQVEVSWEYPDTWSTPHSYFSLTFCVQVQGKSSSEKKDRVFTDKTSATVICRKNASISVRAQDRYYSSSWSEWASIPCS

SEQ ID NO: 195 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSASEKKDRVFTDKTSATVICRKNASISVRAQDRYYSSSWSEWASIPCS

SEQ ID NO: 196 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSSSEKKDRVFTDKTSATVICRKNASISVRAQDRYYSSSWSEWASIPCS

SEQ ID NO: 197 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSASEKKDRVFTDKTSATVICRKNASISVRAQDRYYSSSWSEWASVPCS

SEQ ID NO: 198 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSSSEKKDRVFTDKTSATVICRKNASISVRAQDRYYSSSWSEWASVPCS

SEQ ID NO: 199 - protein, 24 aa, synthetic construct
MDPKGSLSWRILLFLSLAFELSYG

SEQ ID NO: 200 - protein, 20 aa, synthetic construct
MSVPTQVLGLLLLWLTDARC

SEQ ID NO: 201 - protein, 19 aa, synthetic construct
MEWSWVFLFFLSVTTGVHS

SEQ ID NO: 202 - protein, 4 aa, synthetic construct
GGGG

SEQ ID NO: 203 - protein, 10 aa, synthetic construct
GGGGSGGGGS

SEQ ID NO: 204 - protein, 5 aa, synthetic construct
GGGGS

SEQ ID NO: 205 - protein, 25 aa, synthetic construct
  VARIANT 1..25: note = This sequence may encompass 1-5 GGGGS repeating units
GGGGSGGGGSGGGGSGGGGSGGGGS

SEQ ID NO: 206 - protein, 15 aa, synthetic construct
GGGGSGGGGSGGGGS

SEQ ID NO: 207 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 208 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 209 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 210 - protein, 227 aa, synthetic construct
DKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - protein, 227 aa, synthetic construct
DKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 212 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSWLTVDKSRWQQGNVFSCSVMHEALHNHYTQKSLSLSPGK

SEQ ID NO: 213 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGK

SEQ ID NO: 214 - protein, 227 aa, synthetic construct
DKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSWLTVDKSRWQQGNVFSCSVMHEALHNHYTQKSLSLSPGK

SEQ ID NO: 215 - protein, 227 aa, synthetic construct
DKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGK

SEQ ID NO: 216 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDWLTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVSTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 227 aa, synthetic construct
DKTHTCPPCPAPEAAGGPSVFLFPPKPKDTLYIYREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDWLTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 227 aa, synthetic construct
DKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVSTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 221 - protein, 227 aa, synthetic construct
DKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 222 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 223 - DNA, 1374 bp, synthetic construct
  source 1..1374: mol_type = other DNA
atggaccctaagggctctctgagctggaggattctgctgtttctgagcctcgcctttgagctgagctacggcactccagatcccggcatgttcccttgtctgcaccacagccagaatctgctgagggctgtgagcaatatgctgcagaaggccagacagacactcgagttctacccttgcacaagcgaggagagcgaccacgaagacatcacaaaggataagactagcactgtcgaggcttgtctccctctggagctgacaaagaacgagagctgcctcaactctagggagacaagcttcatcactaacggcagctgtctggcttccagaaagactagcttcatgatggctctgtgtctgagcagcatctacgaggatctgaagatgtaccaagtggagttcaaaactatgaacgccaagctgctcatggaccctaagaggcagatctttctggaccagaacatgctggccgtcatcgacgagctgatgcaagccctcaatttcaacagcgagactgtccctcagaagtcctctctggaggagccagacttctataagactaaaatcaaactctgtattctgctgcacgccttcagaatcagagccgtgacaatcgatagggtgatgagctatctgaatgctagcggcggaggaggcagcggaggaggcggaagcggcggaggcggctccgacaagactcacacatgtccaccttgtccagccccagaggctgctggaggaccttccgtctttctgttcccacctaagcctaaggacacactcatgatctctaggacaccagaggtgacatgtgtggtcgtgtccgtcagccacgaggacccagaggtgaagtttaactggtacgtggacggcgtggaagtgcacaatgccaagacaaagccaagggaggagcaatacaacagcacttatagggtcgtgagcgtgctgacagtgctgcatcaagattggctgaacggcaaggagtacaagtgcaaggtgagcaacaaagctctgccagcccctatcgagaagactatcagcaaggccaagggccagcctagggagccacaagtgtacacactgcctccttgtagggacgagctgactaagaaccaagtgtctctgtggtgtctggtcaagggcttttacccttccgatatcgccgtggagtgggaatccaacggccagccagagaataactacaagacaactcctccagtgctggactccgatggctccttcttcctctacagcaagctgactgtggataagagcagatggcagcaaggcaacgtgttcagctgtagcgtgatgcatgaggctctgcacaaccactacactcagaagtctctgtctctgtcccccggcaaatga

SEQ ID NO: 224 - DNA, 1374 bp, synthetic construct
  source 1..1374: mol_type = other DNA
atggaccctaagggctctctgagctggaggattctgctgtttctgagcctcgcctttgagctgagctacggcactccagatcccggcatgttcccttgtctgcaccacagccagaatctgctgagggctgtgagcaatatgctgcagaaggccagacagacactcgagttctacccttgcacaagcgaggagatcgaccacagagacatcacaaaggataagactagcactgtcgaggcttgtctccctctggagctgacaaagaacgagagctgcctcaactctagggagacaagcttcatcactaacggcagctgtctggcttccagaaagactagcttcatgatggctctgtgtctgagcagcatctacgaggatctgaagatgtaccaagtggagttcaaaactatgaacgccaagctgctcatggaccctaagaggcagatctttctggaccagaacatgctggccgtcatcgacgagctgatgcaagccctcaatttcaacagcgagactgtccctcagaagtcctctctggaggagccagacttctataagactaaaatcaaactctgtattctgctgcacgccttcagaatcagagccgtgacaatcgatagggtgatgagctatctgaatgctagcggcggaggaggcagcggaggaggcggaagcggcggaggcggctccgacaagactcacacatgtccaccttgtccagccccagaggctgctggaggaccttccgtctttctgttcccacctaagcctaaggacacactcatgatctctaggacaccagaggtgacatgtgtggtcgtgtccgtcagccacgaggacccagaggtgaagtttaactggtacgtggacggcgtggaagtgcacaatgccaagacaaagccaagggaggagcaatacaacagcacttatagggtcgtgagcgtgctgacagtgctgcatcaagattggctgaacggcaaggagtacaagtgcaaggtgagcaacaaagctctgccagcccctatcgagaagactatcagcaaggccaagggccagcctagggagccacaagtgtacacactgcctccttgtagggacgagctgactaagaaccaagtgtctctgtggtgtctggtcaagggcttttacccttccgatatcgccgtggagtgggaatccaacggccagccagagaataactacaagacaactcctccagtgctggactccgatggctccttcttcctctacagcaagctgactgtggataagagcagatggcagcaaggcaacgtgttcagctgtagcgtgatgcatgaggctctgcacaaccactacactcagaagtctctgtctctgtcccccggcaaatga

SEQ ID NO: 225 - DNA, 1374 bp, synthetic construct
  source 1..1374: mol_type = other DNA
atggaccctaagggctctctgagctggaggattctgctgtttctgagcctcgcttttgagctcagctatggcactccagatcccggcatgttcccttgtctgcaccacagccagaatctgctgagggccgtgagcaatatgctgcagaaagctaggcagacactggagttctacccttgcactagcgaggagatcgatcacgaggatatcacaaaggacaagacatccactgtggaggcttgtctgccactggagctcactaagaacgagagctgtctgaactctagggaaacaagcttcatcactaatggcagctgtctggcttctaggaagacatccttcatgatggctctgtgtctgagctccatctacgaggatctgaagatgtaccaagtggagttcaagacaatgaacgccaagctgctgatggaccctaagagacagatctttctggaccagaacatgctggccgtcatcgatgaactgatgcaagctctgaacttcaacagcgagacagtgcctcagaagagctctctggaggagccaaagttctacaagacaaagattaaactgtgtattctgctgcacgcctttaggattagggctgtcactatcgatagggtgatgagctatctgaatgctagcggcggaggaggcagcggaggaggaggaagcggaggaggcggaagcgacaagactcacacatgtccaccttgtccagccccagaggctgctggaggaccttccgtctttctgttcccacctaagcctaaggacacactcatgatctctaggacaccagaggtgacatgtgtggtcgtgtccgtgagccacgaggacccagaggtgaagttcaactggtacgtggacggcgtggaagtgcacaatgccaagacaaagccaagggaggagcaatacaacagcacttatagggtggtgagcgtgctgacagtgctgcatcaagattggctgaacggcaaggagtacaaatgcaaggtgagcaataaggctctgccagcccctatcgagaagactatctccaaggctaagggccagcctagggagccacaagtgtacactctgcctccttgtagggatgagctgactaagaaccaagtgtctctgtggtgtctggtgaaaggattctaccctagcgacatcgccgtcgagtgggagtccaacggccagccagagaacaactacaagacaacaccaccagtgctggactccgatggctccttctttctgtacagcaagctgactgtcgacaagtctaggtggcagcaaggcaacgtgttcagctgtagcgtgatgcatgaggctctgcacaatcactacacacagaagtctctgtctctgagccccggcaaatga

SEQ ID NO: 226 - DNA, 1374 bp, synthetic construct
  source 1..1374: mol_type = other DNA
atggaccctaagggctctctgagctggaggattctgctgtttctgagcctcgcttttgagctcagctatggcactccagatcccggcatgttcccttgtctgcaccacagccagaatctgctgagggccgtgagcaatatgctgcagaaagctaggcagacactggagttctacccttgcactagcgaggagatcgatcacgaggatatcacaaaggacaagacatccactgtggaggcttgtctgccactggagctcactaagaacgagagctgtctgaactctagggaaacaagcttcatcactaatggcagctgtctggcttctaggaagacatccttcatgatggctctgtgtctgagctccatctacgaggatctgaagatgtaccaagtggagttcaagacaatgaacgccaagctgctgatggaccctaagagacagatctttctggaccagaacatgctggccgtcatcgatgaactgatgcaagctctgaacttcaacagcgagacagtgcctcagaagagctctctggaggagccaaggttctacaagacaaagattaaactgtgtattctgctgcacgcctttaggattagggctgtcactatcgatagggtgatgagctatctgaatgctagcggcggaggaggcagcggaggaggaggaagcggaggaggcggaagcgacaagactcacacatgtccaccttgtccagccccagaggctgctggaggaccttccgtctttctgttcccacctaagcctaaggacacactcatgatctctaggacaccagaggtgacatgtgtggtcgtgtccgtgagccacgaggacccagaggtgaagttcaactggtacgtggacggcgtggaagtgcacaatgccaagacaaagccaagggaggagcaatacaacagcacttatagggtggtgagcgtgctgacagtgctgcatcaagattggctgaacggcaaggagtacaaatgcaaggtgagcaataaggctctgccagcccctatcgagaagactatctccaaggctaagggccagcctagggagccacaagtgtacactctgcctccttgtagggatgagctgactaagaaccaagtgtctctgtggtgtctggtgaaaggattctaccctagcgacatcgccgtcgagtgggagtccaacggccagccagagaacaactacaagacaacaccaccagtgctggactccgatggctccttctttctgtacagcaagctgactgtcgacaagtctaggtggcagcaaggcaacgtgttcagctgtagcgtgatgcatgaggctctgcacaatcactacacacagaagtctctgtctctgagccccggcaaatga

SEQ ID NO: 227 - DNA, 1374 bp, synthetic construct
  source 1..1374: mol_type = other DNA
atggaccctaagggctctctgagctggaggattctgctgtttctgagcctcgcttttgagctcagctatggcactccagatcccggcatgttcccttgtctgcaccacagccagaatctgctgagggccgtgagcaatatgctgcagaaagctaggcagacactggagttctacccttgcactagcgaggagatcgatcacgaggatatcacaaaggacaagacatccactgtggaggcttgtctgccactggagctcactaagaacgagagctgtctgaactctagggaaacaagcttcatcactaatggcagctgtctggcttctaggaagacatccttcatgatggctctgtgtctgagctccatctacgaggatctgaagatgtaccaagtggagttcaagacaatgaacgccaagctgctgatggaccctaagagacagatctttctggaccagaacatgctggccgtcatcgatgaactgatgcaagctctgaacttcaacagcgagacagtgcctcagaagagctctctggaggagccagacttcagcaagacaaagattaaactgtgtattctgctgcacgcctttaggattagggctgtcactatcgatagggtgatgagctatctgaatgctagcggcggaggaggcagcggaggaggaggaagcggaggaggcggaagcgacaagactcacacatgtccaccttgtccagccccagaggctgctggaggaccttccgtctttctgttcccacctaagcctaaggacacactcatgatctctaggacaccagaggtgacatgtgtggtcgtgtccgtgagccacgaggacccagaggtgaagttcaactggtacgtggacggcgtggaagtgcacaatgccaagacaaagccaagggaggagcaatacaacagcacttatagggtggtgagcgtgctgacagtgctgcatcaagattggctgaacggcaaggagtacaaatgcaaggtgagcaataaggctctgccagcccctatcgagaagactatctccaaggctaagggccagcctagggagccacaagtgtacactctgcctccttgtagggatgagctgactaagaaccaagtgtctctgtggtgtctggtgaaaggattctaccctagcgacatcgccgtcgagtgggagtccaacggccagccagagaacaactacaagacaacaccaccagtgctggactccgatggctccttctttctgtacagcaagctgactgtcgacaagtctaggtggcagcaaggcaacgtgttcagctgtagcgtgatgcatgaggctctgcacaatcactacacacagaagtctctgtctctgagccccggcaaatga

SEQ ID NO: 228 - DNA, 1374 bp, synthetic construct
  source 1..1374: mol_type = other DNA
atggaccctaagggctctctgagctggaggattctgctgtttctgagcctcgcttttgagctcagctatggcactccagatcccggcatgttcccttgtctgcaccacagccagaatctgctgagggccgtgagcaatatgctgcagaaagctaggcagacactggagttctacccttgcactagcgaggagatcgatcacgaggatatcacaaaggacaagacatccactgtggaggcttgtctgccactggagctcactaagaacgagagctgtctgaactctagggaaacaagcttcatcactaatggcagctgtctggcttctaggaagacatccttcatgatggctctgtgtctgagctccatctacgaggatctgaagatgtaccaagtggagttcaagacaatgaacgccaagctgctgatggaccctaagagacagatctttctggaccagaacatgctggccgtcatcgatgaactgatgcaagctctgaacttcaacagcgagacagtgcctcagaagagctctctggaggagccagacttcactaagacaaagattaaactgtgtattctgctgcacgcctttaggattagggctgtcactatcgatagggtgatgagctatctgaatgctagcggcggaggaggcagcggaggaggaggaagcggaggaggcggaagcgacaagactcacacatgtccaccttgtccagccccagaggctgctggaggaccttccgtctttctgttcccacctaagcctaaggacacactcatgatctctaggacaccagaggtgacatgtgtggtcgtgtccgtgagccacgaggacccagaggtgaagttcaactggtacgtggacggcgtggaagtgcacaatgccaagacaaagccaagggaggagcaatacaacagcacttatagggtggtgagcgtgctgacagtgctgcatcaagattggctgaacggcaaggagtacaaatgcaaggtgagcaataaggctctgccagcccctatcgagaagactatctccaaggctaagggccagcctagggagccacaagtgtacactctgcctccttgtagggatgagctgactaagaaccaagtgtctctgtggtgtctggtgaaaggattctaccctagcgacatcgccgtcgagtgggagtccaacggccagccagagaacaactacaagacaacaccaccagtgctggactccgatggctccttctttctgtacagcaagctgactgtcgacaagtctaggtggcagcaaggcaacgtgttcagctgtagcgtgatgcatgaggctctgcacaatcactacacacagaagtctctgtctctgagccccggcaaatga

SEQ ID NO: 229 - DNA, 1374 bp, synthetic construct
  source 1..1374: mol_type = other DNA
atggaccctaagggctctctgagctggaggattctgctgtttctgagcctcgcttttgagctcagctatggcactccagatcccggcatgttcccttgtctgcaccacagccagaatctgctgagggccgtgagcaatatgctgcagaaagctaggcagacactggagttctacccttgcactagcgaggagatcgatcacgaggatatcacaaaggacaagacatccactgtggaggcttgtctgccactggagctcactaagaacgagagctgtctgaactctagggaaacaagcttcatcactaatggcagctgtctggcttctaggaagacatccttcatgatggctctgtgtctgagctccatctacgaggatctgaagatgtaccaagtggagttcaagacaatgaacgccaagctgctgatggaccctaagagacagatctttctggaccagaacatgctggccgtcatcgatgaactgatgcaagctctgaacttcaacagcgagacagtgcctcagaagagctctctggaggagccagacttcgctaagacaaagattaaactgtgtattctgctgcacgcctttaggattagggctgtcactatcgatagggtgatgagctatctgaatgctagcggcggaggaggcagcggaggaggaggaagcggaggaggcggaagcgacaagactcacacatgtccaccttgtccagccccagaggctgctggaggaccttccgtctttctgttcccacctaagcctaaggacacactcatgatctctaggacaccagaggtgacatgtgtggtcgtgtccgtgagccacgaggacccagaggtgaagttcaactggtacgtggacggcgtggaagtgcacaatgccaagacaaagccaagggaggagcaatacaacagcacttatagggtggtgagcgtgctgacagtgctgcatcaagattggctgaacggcaaggagtacaaatgcaaggtgagcaataaggctctgccagcccctatcgagaagactatctccaaggctaagggccagcctagggagccacaagtgtacactctgcctccttgtagggatgagctgactaagaaccaagtgtctctgtggtgtctggtgaaaggattctaccctagcgacatcgccgtcgagtgggagtccaacggccagccagagaacaactacaagacaacaccaccagtgctggactccgatggctccttctttctgtacagcaagctgactgtcgacaagtctaggtggcagcaaggcaacgtgttcagctgtagcgtgatgcatgaggctctgcacaatcactacacacagaagtctctgtctctgagccccggcaaatga

SEQ ID NO: 230 - DNA, 1686 bp, synthetic construct
  source 1..1686: mol_type = other DNA
atggaccctaagggctctctgagctggaggattctgctctttctctctctggcctttgaactgagctacggaatctgggagctgaagaaagacgtctacgtggtcgagctggactggtacccagatgcccccggcgagatggtcgtcctcacatgcgacacaccagaggaagacggcatcacttggactctcgatcagtcctccgaagtgctgggctccggaaagacactgacaatccaagtgaaggagttcggcgatgctggacagtacacttgccacaaaggaggagaggtgctgagccactctctgctgctgctgcacaagaaagaggacggaatctggtccacagacattctcaaggaccaaaaggagcctaagaacaagacatttctgaggtgcgaggccaagaactacagcggcagattcacttgctggtggctcacaacaatcagcacagacctcacattctccgtgaagtcctccagaggcagctccgacccacaaggcgtgacttgtggagctgctacactcagcgccgaaagggtgaggggagacaataaggagtacgagtatagcgtcgagtgccaagaggatagcgcttgtccagctgccgaagagtctctgccaatcgaggtcatggtggatgccgtccacaagctcaaatacgagaattacactagctccttcttcattagggacattattaagccagaccctcctaagaacctccagctgaagccactcaagaactctaggcaagtggaggtgagctgggagtatccagatacttggtccacacctcactcctacttcagcctcacattctgcgtccaagtgcaaggcaagtccaagagagagaagaaggatagggtcttcacagataagacaagcgccacagtgatctgtaggaagaacgcctccatcagcgtgagggcccaagataggtactattcctccagctggagcgagtgggctagcgtcccttgcagcggaggaggcggagacaaaactcatacttgcccaccttgcccagccccagaagctgctggcggaccatccgtgtttctgttcccacctaagcctaaggacactctgatgatctctaggacaccagaggtgacttgtgtggtggtgagcgtgagccatgaagatccagaggtgaagttcaactggtacgtcgatggcgtggaggtgcataacgccaagacaaaaccaagggaggaacagtacaacagcacatatagggtggtcagcgtcctcactgtgctccaccaagattggctcaatggcaaagaatacaagtgtaaggtctccaacaaagctctgccagctccaatcgagaagactatctccaaggccaagggccaacctagggagccacaagtgtgcactctgccaccttctagggacgaactgactaagaaccaagtgtctctgagctgcgccgtgaagggattttacccttccgatatcgccgtggagtgggagagcaatggccaaccagagaataattataagacaactcctccagtgctggactccgacggcagcttctttctggtcagcaagctgactgtggacaagtctaggtggcagcaaggaaacgtgttcagctgttccgtgatgcatgaagctctgcacaaccactacacacagaagagcctcagcctcagccccggcaagtga

SEQ ID NO: 231 - DNA, 1686 bp, synthetic construct
  source 1..1686: mol_type = other DNA
atggaccctaagggcagcctcagctggaggattctgctgtttctgtctctggcctttgagctgagctatggcatttgggagctgaagaaggacgtgtacgtggtggagctggattggtatccagatgcccccggcgagatggtggtgctgacatgtgacactccagaggaagacggcatcacttggactctcgatcagtcctccgaagtgctgggctccggaaagacactgacaatccaagtgaaggagttcggagatgctggccagtacacttgccacaaaggcggcgaagtgctgagccactctctgctgctgctgcataagaaggagctgggaatctggtccacagacatcctcaaggaccaaaaggagccaaaaaacaagacatttctgaggtgtgaggccaagaactactccggaaggtttacatgctggtggctcacaacaatctccacagacctcactttcagcgtgaagagcagcagaggcagcagcgacccacaaggcgtgacttgtggagccgccactctgtccgctgagagggtgaggggcgacaacaaggagtacgagtactccgtggagtgccaagaggatagcgcttgtccagccgctgaggaaagcctccctatcgaggtgatggtggacgccgtgcacaagctgaagtatgaaaattacacatccagcttcttcattagggacatcatcaagccagatcctccaaagaacctccagctcaagcctctgaagaactctaggcaagtggaggtcagctgggagtatccagacacttggagcacacctcactcctacttctctctgacattctgcgtccaagtgcaaggcaagtccaagagagagaaaaaggatagggtgttcactgacaagactagcgccacagtgatctgcagaaagaacgccagcatcagcgtgagagcccaagataggtactattcctccagctggagcgagtgggcttccgtgccttgtagcggcggaggaggcgacaagactcacacttgtccaccatgcccagccccagaagctgccggaggccctagcgtgtttctgttccctccaaagcctaaggacacactgatgatctctaggactccagaagtcacatgtgtcgtggtgtccgtcagccacgaagatccagaggtcaagttcaactggtacgtggatggagtcgaggtgcacaatgccaagacaaagccaagggaggagcagtacaacagcacatatagggtggtcagcgtgctgactgtgctccaccaagattggctgaacggcaaggagtacaagtgcaaggtcagcaacaaggctctgccagctccaatcgagaagactatctccaaggccaagggccagccaagggagccacaagtgtgcacactgcctccatctagggacgaactgacaaagaaccaagtgtctctgagctgtgctgtgaagggcttctacccaagcgacatcgccgtcgagtgggagagcaatggccagccagagaataactacaaaacaacacctccagtgctggacagcgatggcagcttcttcctcgtcagcaagctgacagtggataagtccagatggcagcaaggcaacgtcttcagctgctccgtgatgcacgaggctctgcacaatcactacactcagaagtctctgtctctgtcccccggcaagtga

SEQ ID NO: 232 - DNA, 1686 bp, synthetic construct
  source 1..1686: mol_type = other DNA
atggaccctaagggctctctgagctggaggattctgctgtttctgtctctggctttcgagctcagctacggaatctgggaactgaagaaggacgtctacgtcgtggagctggattggtatccagatgctcccggcgaaatggtggtgctcacatgcgatactccagaagaggacggcatcacatggacactggatcagagcagcgaagtgctgggctccggaaagacactgactatccaagtgaaggagtttggcgatgctggccagtacacatgtcacaagggcggagaggtgctgagccactctctgctgctgctgcacaagaaggaggatggcatctggtccacagacattctgaaggaccagaaggagcctaagaacgagacatttctgaggtgcgaagccaagaattattccggaaggtttacatgctggtggctgacaacaatcagcacagacctcactttcagcgtgaagtcctctaggggcagcagcgaccctcaaggagtcacatgcggcgccgctactctgtccgccgaaagggtgaggggcgacaacaaagagtacgagtacagcgtggagtgtcaagaggatagcgcttgtccagccgctgaagagtctctgccaatcgaggtcatggtcgatgccgtgcacaagctcaagtacgaaaactacactagctccttcttcatcagagatatcatcaagccagatccaccaaagaacctccagctgaagcctctgaagaactctaggcaagtggaggtgagctgggagtatccagatacttggtccactcctcacagctacttctctctgactttctgtgtccaagtgcaaggcaagtccaagagagagaagaaggatagggtcttcacagacaagacaagcgctactgtgatctgtaggaagaacgcctccatcagcgtgagggcccaagataggtactacagctccagctggagcgagtgggcttccgtgccatgtagcggaggcggaggcgacaagacacatacttgcccaccttgtccagccccagaagctgctggcggccctagcgtgtttctgttccctcctaagcctaaggacacactgatgatctctaggacaccagaggtgacttgtgtggtggtgagcgtgagccatgaagatccagaggtgaagttcaactggtacgtcgatggcgtggaggtgcataacgccaagacaaagcctagggaggagcagtacaacagcacatatagggtggtgtccgtgctgacagtgctgcaccaagattggctcaacggcaaagagtacaagtgtaaggtgagcaacaaggccctcccagcccctatcgagaagacaatctccaaggctaagggccagccaagggaaccacaagtgtgcacactccctccttctagggacgagctgactaagaatcaagtgagcctcagctgtgccgtgaagggattctacccaagcgacattgctgtggagtgggagtccaacggccagccagagaacaactataagacaacaccaccagtgctggatagcgatggcagcttctttctggtgtccaaactgacagtggacaagtctaggtggcagcaaggcaacgtgttcagctgtagcgtgatgcacgaggccctccacaaccactacacacagaagtctctgagcctctcccccggcaagtga

SEQ ID NO: 233 - DNA, 1686 bp, synthetic construct
  source 1..1686: mol_type = other DNA
atggaccctaagggctctctgagctggaggattctgctgtttctgtctctggctttcgagctcagctacggaatctgggaactgaagaaggacgtctacgtcgtggagctggattggtatccagatgctcccggcgaaatggtggtgctcacatgcgatactccagaagaggacggcatcacatggacactggatcagagcagcgaagtgctgggctccggaaagacactgactatccaagtgaaggagtttggcgatgctggccagtacacatgtcacaagggcggagaggtgctgagccactctctgctgctgctgcacaagaaggaggatggcatctggtccacagacattctgaaggaccagaaggagcctaagaacgacacatttctgaggtgcgaagccaagaattattccggaaggtttacatgctggtggctgacaacaatcagcacagacctcactttcagcgtgaagtcctctaggggcagcagcgaccctcaaggagtcacatgcggcgccgctactctgtccgccgaaagggtgaggggcgacaacaaagagtacgagtacagcgtggagtgtcaagaggatagcgcttgtccagccgctgaagagtctctgccaatcgaggtcatggtcgatgccgtgcacaagctcaagtacgaaaactacactagctccttcttcatcagagatatcatcaagccagatccaccaaagaacctccagctgaagcctctgaagaactctaggcaagtggaggtgagctgggagtatccagatacttggtccactcctcacagctacttctctctgactttctgtgtccaagtgcaaggcaagtccaagagagagaagaaggatagggtcttcacagacaagacaagcgctactgtgatctgtaggaagaacgcctccatcagcgtgagggcccaagataggtactacagctccagctggagcgagtgggcttccgtgccatgtagcggaggcggaggcgacaagacacatacttgcccaccttgtccagccccagaagctgctggcggccctagcgtgtttctgttccctcctaagcctaaggacacactgatgatctctaggacaccagaggtgacttgtgtggtggtgagcgtgagccatgaagatccagaggtgaagttcaactggtacgtcgatggcgtggaggtgcataacgccaagacaaagcctagggaggagcagtacaacagcacatatagggtggtgtccgtgctgacagtgctgcaccaagattggctcaacggcaaagagtacaagtgtaaggtgagcaacaaggccctcccagcccctatcgagaagacaatctccaaggctaagggccagccaagggaaccacaagtgtgcacactccctccttctagggacgagctgactaagaatcaagtgagcctcagctgtgccgtgaagggattctacccaagcgacattgctgtggagtgggagtccaacggccagccagagaacaactataagacaacaccaccagtgctggatagcgatggcagcttctttctggtgtccaaactgacagtggacaagtctaggtggcagcaaggcaacgtgttcagctgtagcgtgatgcacgaggccctccacaaccactacacacagaagtctctgagcctctcccccggcaagtga

SEQ ID NO: 234 - DNA, 1374 bp, synthetic construct
  source 1..1374: mol_type = other DNA
atggaccctaagggctctctgagctggaggattctgctgtttctgagcctcgcttttgagctcagctatggcactccagatcccggcatgttcccttgtctgcaccacagccagaatctgctgagggccgtgagcaatatgctgcagaaagctaggcagacactggagttctacccttgcactagcgaggagatcgatcacgaggatatcacaaaggacaagacatccactgtggaggcttgtctgccactggagctcactaagaacgagagctgtctgaactctagggaaacaagcttcatcactaatggcagctgtctggcttctaggaagacatccttcatgatggctctgtgtctgagctccatctacgaggatctgaagatgtaccaagtggagttcaagacaatgaacgccaagctgctgatggaccctaagagacagatctttctggaccagaacatgctggccgtcatcgatgaactgatgcaagctctgaacttcaacagcgagacagtgcctcagaagagctctctggaggagccagacttcagcaagacaaagattaaactgtgtattctgctgcacgcctttaggattagggctgtcactatcgatagggtgatgagctatctgaatgctagcggcggaggaggcagcggaggaggaggaagcggaggaggcggaagcgacaagactcacacatgtccaccttgtccagccccagaggctgctggaggaccttccgtctttctgttcccacctaagcctaaggacacactctacatcaccagggagccagaggtgacatgtgtggtcgtgtccgtgagccacgaggacccagaggtgaagttcaactggtacgtggacggcgtggaagtgcacaatgccaagacaaagccaagggaggagcaatacaacagcacttatagggtggtgagcgtgctgacagtgctgcatcaagattggctgaacggcaaggagtacaaatgcaaggtgagcaataaggctctgccagcccctatcgagaagactatctccaaggctaagggccagcctagggagccacaagtgtacactctgcctccttgtagggatgagctgactaagaaccaagtgtctctgtggtgtctggtgaaaggattctaccctagcgacatcgccgtcgagtgggagtccaacggccagccagagaacaactacaagacaacaccaccagtgctggactccgatggctccttctttctgtacagcaagctgactgtcgacaagtctaggtggcagcaaggcaacgtgttcagctgtagcgtgatgcatgaggctctgcacaatcactacacacagaagtctctgtctctgagccccggcaaatga

SEQ ID NO: 235 - DNA, 1374 bp, synthetic construct
  source 1..1374: mol_type = other DNA
atggaccctaagggctctctgagctggaggattctgctgtttctgagcctcgcttttgagctcagctatggcactccagatcccggcatgttcccttgtctgcaccacagccagaatctgctgagggccgtgagcaatatgctgcagaaagctaggcagacactggagttctacccttgcactagcgaggagatcgatcacgaggatatcacaaaggacaagacatccactgtggaggcttgtctgccactggagctcactaagaacgagagctgtctgaactctagggaaacaagcttcatcactaatggcagctgtctggcttctaggaagacatccttcatgatggctctgtgtctgagctccatctacgaggatctgaagatgtaccaagtggagttcaagacaatgaacgccaagctgctgatggaccctaagagacagatctttctggaccagaacatgctggccgtcatcgatgaactgatgcaagctctgaacttcaacagcgagacagtgcctcagaagagctctctggaggagccagacttcactaagacaaagattaaactgtgtattctgctgcacgcctttaggattagggctgtcactatcgatagggtgatgagctatctgaatgctagcggcggaggaggcagcggaggaggaggaagcggaggaggcggaagcgacaagactcacacatgtccaccttgtccagccccagaggctgctggaggaccttccgtctttctgttcccacctaagcctaaggacacactctacatcaccagggagccagaggtgacatgtgtggtcgtgtccgtgagccacgaggacccagaggtgaagttcaactggtacgtggacggcgtggaagtgcacaatgccaagacaaagccaagggaggagcaatacaacagcacttatagggtggtgagcgtgctgacagtgctgcatcaagattggctgaacggcaaggagtacaaatgcaaggtgagcaataaggctctgccagcccctatcgagaagactatctccaaggctaagggccagcctagggagccacaagtgtacactctgcctccttgtagggatgagctgactaagaaccaagtgtctctgtggtgtctggtgaaaggattctaccctagcgacatcgccgtcgagtgggagtccaacggccagccagagaacaactacaagacaacaccaccagtgctggactccgatggctccttctttctgtacagcaagctgactgtcgacaagtctaggtggcagcaaggcaacgtgttcagctgtagcgtgatgcatgaggctctgcacaatcactacacacagaagtctctgtctctgagccccggcaaatga

SEQ ID NO: 236 - DNA, 1392 bp, synthetic construct
  source 1..1392: mol_type = other DNA
atggaccctaagggcagcctcagctggaggattctgctgttcctctctctggcctttgaactgagctacggccgcaacctgccggtggcgacaccagatcccggcatgttcccttgcctccatcacagccagaatctgctgagggccgtcagcaatatgctgcagaaggctaggcagacactcgagttctatccttgcacaagcgaggagatcgaccatgaggatatcactaaggacaagactagcactgtggaggcttgtctcccactggaactgactaagaacgagagctgtctgaactctagggagacaagcttcatcactaacggcagctgtctcgcctctaggaagacttccttcatgatggctctgtgcctcagctccatctacgaagatctgaagatgtaccaagtggagtttaaaactatgaacgccaagctgctgatggaccctaagaggcagatctttctcgaccagaacatgctggctgtcatcgacgaactcatgcaagccctcaatttcaacagcgagactgtccctcagaagtcctctctggaggaaccagatttctacaagactaagatcaagctctgtattctgctgcacgcctttaggattagggctgtcacaatcgatagggtcatgagctatctgaatgccagcggcggaggaggcagcggaggaggaggaagcggaggcggcggaagcgacaagactcacacatgccctccttgtccagccccagaagctgccggaggaccttccgtctttctgttcccacctaagcctaaagacacactctacatcaccagggagccagaggtgacatgtgtggtcgtgtccgtcagccacgaggacccagaggtgaagtttaactggtacgtggacggagtggaagtgcacaatgccaagacaaagccaagggaggagcaatacaacagcacttatagggtcgtgagcgtgctgacagtgctgcaccaagattggctcaatggcaaggagtataagtgcaaagtgtccaacaaagctctgccagctcctatcgagaagactatctccaaggccaagggccagcctagggagccacaagtgtatacactgccaccatgtagggacgagctgacaaagaaccaagtgtctctgtggtgtctggtgaagggcttctaccctagcgatattgccgtggagtgggagtccaacggccagccagagaataactacaagacaacacctccagtgctcgattccgacggctccttcttcctctactccaagctgactgtcgacaagtctaggtggcagcaaggcaatgtgttcagctgtagcgtgatgcacgaggctctgcacaatcactatacacagaagtctctgtccctcagccccggcaaatga

SEQ ID NO: 237 - DNA, 1686 bp, synthetic construct
  source 1..1686: mol_type = other DNA
atggaccctaagggctctctgagctggaggattctgctctttctctctctggcctttgaactgagctacggaatctgggagctgaagaaagacgtctacgtggtcgagctggactggtacccagatgcccccggcgagatggtcgtcctcacatgcgacacaccagaggaagacggcatcacttggactctcgatcagtcctccgaagtgctgggctccggaaagacactgacaatccaagtgaaggagttcggcgatgctggacagtacacttgccacaaaggaggagaggtgctgagccactctctgctgctgctgcacaagaaagaggacggaatctggtccacagacattctcaaggaccaaaaggagcctaagaacaagacatttctgaggtgcgaggccaagaactacagcggcagattcacttgctggtggctcacaacaatcagcacagacctcacattctccgtgaagtcctccagaggcagctccgacccacaaggcgtgacttgtggagctgctacactcagcgccgaaagggtgaggggagacaataaggagtacgagtatagcgtcgagtgccaagaggatagcgcttgtccagctgccgaagagtctctgccaatcgaggtcatggtggatgccgtccacaagctcaaatacgagaattacactagctccttcttcattagggacattattaagccagaccctcctaagaacctccagctgaagccactcaagaactctaggcaagtggaggtgagctgggagtatccagatacttggtccacacctcactcctacttcagcctcacattctgcgtccaagtgcaaggcaagtccaagagagagaagaaggatagggtcttcacagataagacaagcgccacagtgatctgtaggaagaacgcctccatcagcgtgagggcccaagataggtactattcctccagctggagcgagtgggctagcgtcccttgcagcggaggaggcggagacaaaactcatacttgcccaccttgcccagccccagaagctgctggcggaccatccgtgtttctgttcccacctaagcctaaggacactctgtacatcaccagggagccagaggtgacttgtgtggtggtgagcgtgagccatgaagatccagaggtgaagttcaactggtacgtcgatggcgtggaggtgcataacgccaagacaaaaccaagggaggaacagtacaacagcacatatagggtggtcagcgtcctcactgtgctccaccaagattggctcaatggcaaagaatacaagtgtaaggtctccaacaaagctctgccagctccaatcgagaagactatctccaaggccaagggccaacctagggagccacaagtgtgcactctgccaccttctagggacgaactgactaagaaccaagtgtctctgagctgcgccgtgaagggattttacccttccgatatcgccgtggagtgggagagcaatggccaaccagagaataattataagacaactcctccagtgctggactccgacggcagcttctttctggtcagcaagctgactgtggacaagtctaggtggcagcaaggaaacgtgttcagctgttccgtgatgcatgaagctctgcacaaccactacacacagaagagcctcagcctcagccccggcaagtga

SEQ ID NO: 238 - protein, 6 aa, synthetic construct
RNLPVA

SEQ ID NO: 239 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 240 - DNA, 1375 bp, synthetic construct
  source 1..1375: mol_type = other DNA
atggaccctaagggctctctgagctggaggattctgctgtttctgagcctcgcctttgagctgagctacggcactccagatcccggcatgttcccttgtctgcaccacagccagaatctgctgagggctgtgagcaatatgctgcagaaggccagacagacactcgagttctacccttgcacaagcgaggagagcgaccacgaagacatcacaaaggataagactagcactgtcgaggcttgtctccctctggagctgacaaagaacgagagctgcctcaactctagggagacaagcttcatcactaacggcagctgtctggcttccagaaagactagcttcatgatggctctgtgtctgagcagcatctacgaggatctgaagatgtaccaagtggagttcaaaactatgaacgccaagctgctcatggaccctaagaggcagatctttctggaccagaacatgctggccgtcatcgacgagctgatgcaagccctcaatttcaacagcgagactgtccctcagaagtcctctctggaggagccagacttctataagactaaaatcaaactctgtattctgctgcacgccttcagaatcagagccgtgacaatcgatagggtgatgagctatctgaatgctagcggcggaggaggcagcggaggaggcggaagcggcggaggcggctccggacaagactcacacatgccctccttgtccagccccagaagctgccggaggaccttccgtctttctgttcccacctaagcctaaagacacactctacatcaccagggagccagaggtgacatgtgtggtcgtgtccgtcagccacgaggacccagaggtgaagtttaactggtacgtggacggagtggaagtgcacaatgccaagacaaagccaagggaggagcaatacaacagcacttatagggtcgtgagcgtgctgacagtgctgcaccaagattggctcaatggcaaggagtataagtgcaaagtgtccaacaaagctctgccagctcctatcgagaagactatctccaaggccaagggccagcctagggagccacaagtgtatacactgccaccatgtagggacgagctgacaaagaaccaagtgtctctgtggtgtctggtgaagggcttctaccctagcgatattgccgtggagtgggagtccaacggccagccagagaataactacaagacaacacctccagtgctcgattccgacggctccttcttcctctactccaagctgactgtcgacaagtctaggtggcagcaaggcaatgtgttcagctgtagcgtgatgcacgaggctctgcacaatcactatacacagaagtctctgtccctcagccccggcaaatga

SEQ ID NO: 241 - DNA, 1374 bp, synthetic construct
  source 1..1374: mol_type = other DNA
atggaccctaagggctctctgagctggaggattctgctgtttctgagcctcgcctttgagctgagctacggcactccagatcccggcatgttcccttgtctgcaccacagccagaatctgctgagggctgtgagcaatatgctgcagaaggccagacagacactcgagttctacccttgcacaagcgaggagatcgaccacagagacatcacaaaggataagactagcactgtcgaggcttgtctccctctggagctgacaaagaacgagagctgcctcaactctagggagacaagcttcatcactaacggcagctgtctggcttccagaaagactagcttcatgatggctctgtgtctgagcagcatctacgaggatctgaagatgtaccaagtggagttcaaaactatgaacgccaagctgctcatggaccctaagaggcagatctttctggaccagaacatgctggccgtcatcgacgagctgatgcaagccctcaatttcaacagcgagactgtccctcagaagtcctctctggaggagccagacttctataagactaaaatcaaactctgtattctgctgcacgccttcagaatcagagccgtgacaatcgatagggtgatgagctatctgaatgctagcggcggaggaggcagcggaggaggcggaagcggcggaggcggctccgacaagactcacacatgccctccttgtccagccccagaagctgccggaggaccttccgtctttctgttcccacctaagcctaaagacacactctacatcaccagggagccagaggtgacatgtgtggtcgtgtccgtcagccacgaggacccagaggtgaagtttaactggtacgtggacggagtggaagtgcacaatgccaagacaaagccaagggaggagcaatacaacagcacttatagggtcgtgagcgtgctgacagtgctgcaccaagattggctcaatggcaaggagtataagtgcaaagtgtccaacaaagctctgccagctcctatcgagaagactatctccaaggccaagggccagcctagggagccacaagtgtatacactgccaccatgtagggacgagctgacaaagaaccaagtgtctctgtggtgtctggtgaagggcttctaccctagcgatattgccgtggagtgggagtccaacggccagccagagaataactacaagacaacacctccagtgctcgattccgacggctccttcttcctctactccaagctgactgtcgacaagtctaggtggcagcaaggcaatgtgttcagctgtagcgtgatgcacgaggctctgcacaatcactatacacagaagtctctgtccctcagccccggcaaatga

SEQ ID NO: 242 - DNA, 1374 bp, synthetic construct
  source 1..1374: mol_type = other DNA
atggaccctaagggctctctgagctggaggattctgctgtttctgagcctcgcttttgagctcagctatggcactccagatcccggcatgttcccttgtctgcaccacagccagaatctgctgagggccgtgagcaatatgctgcagaaagctaggcagacactggagttctacccttgcactagcgaggagatcgatcacgaggatatcacaaaggacaagacatccactgtggaggcttgtctgccactggagctcactaagaacgagagctgtctgaactctagggaaacaagcttcatcactaatggcagctgtctggcttctaggaagacatccttcatgatggctctgtgtctgagctccatctacgaggatctgaagatgtaccaagtggagttcaagacaatgaacgccaagctgctgatggaccctaagagacagatctttctggaccagaacatgctggccgtcatcgatgaactgatgcaagctctgaacttcaacagcgagacagtgcctcagaagagctctctggaggagccaaagttctacaagacaaagattaaactgtgtattctgctgcacgcctttaggattagggctgtcactatcgatagggtgatgagctatctgaatgctagcggcggaggaggcagcggaggaggaggaagcggaggaggcggaagcgacaagactcacacatgccctccttgtccagccccagaagctgccggaggaccttccgtctttctgttcccacctaagcctaaagacacactctacatcaccagggagccagaggtgacatgtgtggtcgtgtccgtcagccacgaggacccagaggtgaagtttaactggtacgtggacggagtggaagtgcacaatgccaagacaaagccaagggaggagcaatacaacagcacttatagggtcgtgagcgtgctgacagtgctgcaccaagattggctcaatggcaaggagtataagtgcaaagtgtccaacaaagctctgccagctcctatcgagaagactatctccaaggccaagggccagcctagggagccacaagtgtatacactgccaccatgtagggacgagctgacaaagaaccaagtgtctctgtggtgtctggtgaagggcttctaccctagcgatattgccgtggagtgggagtccaacggccagccagagaataactacaagacaacacctccagtgctcgattccgacggctccttcttcctctactccaagctgactgtcgacaagtctaggtggcagcaaggcaatgtgttcagctgtagcgtgatgcacgaggctctgcacaatcactatacacagaagtctctgtccctcagccccggcaaatga

SEQ ID NO: 243 - DNA, 1374 bp, synthetic construct
  source 1..1374: mol_type = other DNA
atggaccctaagggctctctgagctggaggattctgctgtttctgagcctcgcttttgagctcagctatggcactccagatcccggcatgttcccttgtctgcaccacagccagaatctgctgagggccgtgagcaatatgctgcagaaagctaggcagacactggagttctacccttgcactagcgaggagatcgatcacgaggatatcacaaaggacaagacatccactgtggaggcttgtctgccactggagctcactaagaacgagagctgtctgaactctagggaaacaagcttcatcactaatggcagctgtctggcttctaggaagacatccttcatgatggctctgtgtctgagctccatctacgaggatctgaagatgtaccaagtggagttcaagacaatgaacgccaagctgctgatggaccctaagagacagatctttctggaccagaacatgctggccgtcatcgatgaactgatgcaagctctgaacttcaacagcgagacagtgcctcagaagagctctctggaggagccaaggttctacaagacaaagattaaactgtgtattctgctgcacgcctttaggattagggctgtcactatcgatagggtgatgagctatctgaatgctagcggcggaggaggcagcggaggaggaggaagcggaggaggcggaagcgacaagactcacacatgccctccttgtccagccccagaagctgccggaggaccttccgtctttctgttcccacctaagcctaaagacacactctacatcaccagggagccagaggtgacatgtgtggtcgtgtccgtcagccacgaggacccagaggtgaagtttaactggtacgtggacggagtggaagtgcacaatgccaagacaaagccaagggaggagcaatacaacagcacttatagggtcgtgagcgtgctgacagtgctgcaccaagattggctcaatggcaaggagtataagtgcaaagtgtccaacaaagctctgccagctcctatcgagaagactatctccaaggccaagggccagcctagggagccacaagtgtatacactgccaccatgtagggacgagctgacaaagaaccaagtgtctctgtggtgtctggtgaagggcttctaccctagcgatattgccgtggagtgggagtccaacggccagccagagaataactacaagacaacacctccagtgctcgattccgacggctccttcttcctctactccaagctgactgtcgacaagtctaggtggcagcaaggcaatgtgttcagctgtagcgtgatgcacgaggctctgcacaatcactatacacagaagtctctgtccctcagccccggcaaatga

SEQ ID NO: 244 - DNA, 1374 bp, synthetic construct
  source 1..1374: mol_type = other DNA
atggaccctaagggctctctgagctggaggattctgctgtttctgagcctcgcttttgagctcagctatggcactccagatcccggcatgttcccttgtctgcaccacagccagaatctgctgagggccgtgagcaatatgctgcagaaagctaggcagacactggagttctacccttgcactagcgaggagatcgatcacgaggatatcacaaaggacaagacatccactgtggaggcttgtctgccactggagctcactaagaacgagagctgtctgaactctagggaaacaagcttcatcactaatggcagctgtctggcttctaggaagacatccttcatgatggctctgtgtctgagctccatctacgaggatctgaagatgtaccaagtggagttcaagacaatgaacgccaagctgctgatggaccctaagagacagatctttctggaccagaacatgctggccgtcatcgatgaactgatgcaagctctgaacttcaacagcgagacagtgcctcagaagagctctctggaggagccagacttcgctaagacaaagattaaactgtgtattctgctgcacgcctttaggattagggctgtcactatcgatagggtgatgagctatctgaatgctagcggcggaggaggcagcggaggaggaggaagcggaggaggcggaagcgacaagactcacacatgccctccttgtccagccccagaagctgccggaggaccttccgtctttctgttcccacctaagcctaaagacacactctacatcaccagggagccagaggtgacatgtgtggtcgtgtccgtcagccacgaggacccagaggtgaagtttaactggtacgtggacggagtggaagtgcacaatgccaagacaaagccaagggaggagcaatacaacagcacttatagggtcgtgagcgtgctgacagtgctgcaccaagattggctcaatggcaaggagtataagtgcaaagtgtccaacaaagctctgccagctcctatcgagaagactatctccaaggccaagggccagcctagggagccacaagtgtatacactgccaccatgtagggacgagctgacaaagaaccaagtgtctctgtggtgtctggtgaagggcttctaccctagcgatattgccgtggagtgggagtccaacggccagccagagaataactacaagacaacacctccagtgctcgattccgacggctccttcttcctctactccaagctgactgtcgacaagtctaggtggcagcaaggcaatgtgttcagctgtagcgtgatgcacgaggctctgcacaatcactatacacagaagtctctgtccctcagccccggcaaatga

SEQ ID NO: 245 - DNA, 1686 bp, synthetic construct
  source 1..1686: mol_type = other DNA
atggaccctaagggcagcctcagctggaggattctgctgtttctgtctctggcctttgagctgagctatggcatttgggagctgaagaaggacgtgtacgtggtggagctggattggtatccagatgcccccggcgagatggtggtgctgacatgtgacactccagaggaagacggcatcacttggactctcgatcagtcctccgaagtgctgggctccggaaagacactgacaatccaagtgaaggagttcggagatgctggccagtacacttgccacaaaggcggcgaagtgctgagccactctctgctgctgctgcataagaaggagctgggaatctggtccacagacatcctcaaggaccaaaaggagccaaaaaacaagacatttctgaggtgtgaggccaagaactactccggaaggtttacatgctggtggctcacaacaatctccacagacctcactttcagcgtgaagagcagcagaggcagcagcgacccacaaggcgtgacttgtggagccgccactctgtccgctgagagggtgaggggcgacaacaaggagtacgagtactccgtggagtgccaagaggatagcgcttgtccagccgctgaggaaagcctccctatcgaggtgatggtggacgccgtgcacaagctgaagtatgaaaattacacatccagcttcttcattagggacatcatcaagccagatcctccaaagaacctccagctcaagcctctgaagaactctaggcaagtggaggtcagctgggagtatccagacacttggagcacacctcactcctacttctctctgacattctgcgtccaagtgcaaggcaagtccaagagagagaaaaaggatagggtgttcactgacaagactagcgccacagtgatctgcagaaagaacgccagcatcagcgtgagagcccaagataggtactattcctccagctggagcgagtgggcttccgtgccttgtagcggcggaggaggcgacaaaactcatacttgcccaccttgcccagccccagaagctgctggcggaccatccgtgtttctgttcccacctaagcctaaggacactctgtacatcaccagggagccagaggtgacttgtgtggtggtgagcgtgagccatgaagatccagaggtgaagttcaactggtacgtcgatggcgtggaggtgcataacgccaagacaaaaccaagggaggaacagtacaacagcacatatagggtggtcagcgtcctcactgtgctccaccaagattggctcaatggcaaagaatacaagtgtaaggtctccaacaaagctctgccagctccaatcgagaagactatctccaaggccaagggccaacctagggagccacaagtgtgcactctgccaccttctagggacgaactgactaagaaccaagtgtctctgagctgcgccgtgaagggattttacccttccgatatcgccgtggagtgggagagcaatggccaaccagagaataattataagacaactcctccagtgctggactccgacggcagcttctttctggtcagcaagctgactgtggacaagtctaggtggcagcaaggaaacgtgttcagctgttccgtgatgcatgaagctctgcacaaccactacacacagaagagcctcagcctcagccccggcaagtga

SEQ ID NO: 246 - DNA, 1686 bp, synthetic construct
  source 1..1686: mol_type = other DNA
atggaccctaagggctctctgagctggaggattctgctgtttctgtctctggctttcgagctcagctacggaatctgggaactgaagaaggacgtctacgtcgtggagctggattggtatccagatgctcccggcgaaatggtggtgctcacatgcgatactccagaagaggacggcatcacatggacactggatcagagcagcgaagtgctgggctccggaaagacactgactatccaagtgaaggagtttggcgatgctggccagtacacatgtcacaagggcggagaggtgctgagccactctctgctgctgctgcacaagaaggaggatggcatctggtccacagacattctgaaggaccagaaggagcctaagaacgagacatttctgaggtgcgaagccaagaattattccggaaggtttacatgctggtggctgacaacaatcagcacagacctcactttcagcgtgaagtcctctaggggcagcagcgaccctcaaggagtcacatgcggcgccgctactctgtccgccgaaagggtgaggggcgacaacaaagagtacgagtacagcgtggagtgtcaagaggatagcgcttgtccagccgctgaagagtctctgccaatcgaggtcatggtcgatgccgtgcacaagctcaagtacgaaaactacactagctccttcttcatcagagatatcatcaagccagatccaccaaagaacctccagctgaagcctctgaagaactctaggcaagtggaggtgagctgggagtatccagatacttggtccactcctcacagctacttctctctgactttctgtgtccaagtgcaaggcaagtccaagagagagaagaaggatagggtcttcacagacaagacaagcgctactgtgatctgtaggaagaacgcctccatcagcgtgagggcccaagataggtactacagctccagctggagcgagtgggcttccgtgccatgtagcggaggcggaggcgacaaaactcatacttgcccaccttgcccagccccagaagctgctggcggaccatccgtgtttctgttcccacctaagcctaaggacactctgtacatcaccagggagccagaggtgacttgtgtggtggtgagcgtgagccatgaagatccagaggtgaagttcaactggtacgtcgatggcgtggaggtgcataacgccaagacaaaaccaagggaggaacagtacaacagcacatatagggtggtcagcgtcctcactgtgctccaccaagattggctcaatggcaaagaatacaagtgtaaggtctccaacaaagctctgccagctccaatcgagaagactatctccaaggccaagggccaacctagggagccacaagtgtgcactctgccaccttctagggacgaactgactaagaaccaagtgtctctgagctgcgccgtgaagggattttacccttccgatatcgccgtggagtgggagagcaatggccaaccagagaataattataagacaactcctccagtgctggactccgacggcagcttctttctggtcagcaagctgactgtggacaagtctaggtggcagcaaggaaacgtgttcagctgttccgtgatgcatgaagctctgcacaaccactacacacagaagagcctcagcctcagccccggcaagtga

SEQ ID NO: 247 - DNA, 1686 bp, synthetic construct
  source 1..1686: mol_type = other DNA
atggaccctaagggctctctgagctggaggattctgctgtttctgtctctggctttcgagctcagctacggaatctgggaactgaagaaggacgtctacgtcgtggagctggattggtatccagatgctcccggcgaaatggtggtgctcacatgcgatactccagaagaggacggcatcacatggacactggatcagagcagcgaagtgctgggctccggaaagacactgactatccaagtgaaggagtttggcgatgctggccagtacacatgtcacaagggcggagaggtgctgagccactctctgctgctgctgcacaagaaggaggatggcatctggtccacagacattctgaaggaccagaaggagcctaagaacgacacatttctgaggtgcgaagccaagaattattccggaaggtttacatgctggtggctgacaacaatcagcacagacctcactttcagcgtgaagtcctctaggggcagcagcgaccctcaaggagtcacatgcggcgccgctactctgtccgccgaaagggtgaggggcgacaacaaagagtacgagtacagcgtggagtgtcaagaggatagcgcttgtccagccgctgaagagtctctgccaatcgaggtcatggtcgatgccgtgcacaagctcaagtacgaaaactacactagctccttcttcatcagagatatcatcaagccagatccaccaaagaacctccagctgaagcctctgaagaactctaggcaagtggaggtgagctgggagtatccagatacttggtccactcctcacagctacttctctctgactttctgtgtccaagtgcaaggcaagtccaagagagagaagaaggatagggtcttcacagacaagacaagcgctactgtgatctgtaggaagaacgcctccatcagcgtgagggcccaagataggtactacagctccagctggagcgagtgggcttccgtgccatgtagcggaggcggaggcgacaaaactcatacttgcccaccttgcccagccccagaagctgctggcggaccatccgtgtttctgttcccacctaagcctaaggacactctgtacatcaccagggagccagaggtgacttgtgtggtggtgagcgtgagccatgaagatccagaggtgaagttcaactggtacgtcgatggcgtggaggtgcataacgccaagacaaaaccaagggaggaacagtacaacagcacatatagggtggtcagcgtcctcactgtgctccaccaagattggctcaatggcaaagaatacaagtgtaaggtctccaacaaagctctgccagctccaatcgagaagactatctccaaggccaagggccaacctagggagccacaagtgtgcactctgccaccttctagggacgaactgactaagaaccaagtgtctctgagctgcgccgtgaagggattttacccttccgatatcgccgtggagtgggagagcaatggccaaccagagaataattataagacaactcctccagtgctggactccgacggcagcttctttctggtcagcaagctgactgtggacaagtctaggtggcagcaaggaaacgtgttcagctgttccgtgatgcatgaagctctgcacaaccactacacacagaagagcctcagcctcagccccggcaagtga

SEQ ID NO: 248 - DNA, 1374 bp, synthetic construct
  source 1..1374: mol_type = other DNA
atggaccctaagggcagcctcagctggaggattctgctgttcctctctctggcctttgaactgagctacggcacaccagatcccggcatgttcccttgcctccatcacagccagaatctgctgagggccgtcagcaatatgctgcagaaggctaggcagacactcgagttctatccttgcacaagcgaggagatcgaccatgaggatatcactaaggacaagactagcactgtggaggcttgtctcccactggaactgactaagaacgagagctgtctgaactctagggagacaagcttcatcactaacggcagctgtctcgcctctaggaagacttccttcatgatggctctgtgcctcagctccatctacgaagatctgaagatgtaccaagtggagtttaaaactatgaacgccaagctgctgatggaccctaagaggcagatctttctcgaccagaacatgctggctgtcatcgacgaactcatgcaagccctcaatttcaacagcgagactgtccctcagaagtcctctctggaggaaccagatttctacaagactaagatcaagctctgtattctgctgcacgcctttaggattagggctgtcacaatcgatagggtcatgagctatctgaatgccagcggcggaggaggcagcggaggaggaggaagcggaggcggcggaagcgacaagactcacacatgccctccttgtccagccccagaagctgccggaggaccttccgtctttctgttcccacctaagcctaaagacacactctacatcaccagggagccagaggtgacatgtgtggtcgtgtccgtcagccacgaggacccagaggtgaagtttaactggtacgtggacggagtggaagtgcacaatgccaagacaaagccaagggaggagcaatacaacagcacttatagggtcgtgagcgtgctgacagtgctgcaccaagattggctcaatggcaaggagtataagtgcaaagtgtccaacaaagctctgccagctcctatcgagaagactatctccaaggccaagggccagcctagggagccacaagtgtatacactgccaccatgtagggacgagctgacaaagaaccaagtgtctctgtggtgtctggtgaagggcttctaccctagcgatattgccgtggagtgggagtccaacggccagccagagaataactacaagacaacacctccagtgctcgattccgacggctccttcttcctctactccaagctgactgtcgacaagtctaggtggcagcaaggcaatgtgttcagctgtagcgtgatgcacgaggctctgcacaatcactatacacagaagtctctgtccctcagccccggcaaatga

SEQ ID NO: 249 - DNA, 1374 bp, synthetic construct
  source 1..1374: mol_type = other DNA
atggaccctaagggcagcctcagctggaggattctgctgttcctctctctggcctttgaactgagctacggcacaccagatcccggcatgttcccttgcctccatcacagccagaatctgctgagggccgtcagcaatatgctgcagaaggctaggcagacactcgagttctatccttgcacaagcgaggagatcgaccatgaggatatcactaaggacaagactagcactgtggaggcttgtctcccactggaactgactaagaacgagagctgtctgaactctagggagacaagcttcatcactaacggcagctgtctcgcctctaggaagacttccttcatgatggctctgtgcctcagctccatctacgaagatctgaagatgtaccaagtggagtttaaaactatgaacgccaagctgctgatggaccctaagaggcagatctttctcgaccagaacatgctggctgtcatcgacgaactcatgcaagccctcaatttcaacagcgagactgtccctcagaagtcctctctggaggaaccagatttctacaagactaagatcaagctctgtattctgctgcacgcctttaggattagggctgtcacaatcgatagggtcatgagctatctgaatgccagcggcggaggaggcagcggaggaggaggaagcggaggcggcggaagcgacaagactcacacatgtccaccttgtccagccccagaggctgctggaggaccttccgtctttctgttcccacctaagcctaaggacacactcatgatctctaggacaccagaggtgacatgtgtggtcgtgtccgtcagccacgaggacccagaggtgaagtttaactggtacgtggacggcgtggaagtgcacaatgccaagacaaagccaagggaggagcaatacaacagcacttatagggtcgtgagcgtgctgacagtgctgcatcaagattggctgaacggcaaggagtacaagtgcaaggtgagcaacaaagctctgccagcccctatcgagaagactatcagcaaggccaagggccagcctagggagccacaagtgtacacactgcctccttgtagggacgagctgactaagaaccaagtgtctctgtggtgtctggtcaagggcttttacccttccgatatcgccgtggagtgggaatccaacggccagccagagaataactacaagacaactcctccagtgctggactccgatggctccttcttcctctacagcaagctgactgtggataagagcagatggcagcaaggcaacgtgttcagctgtagcgtgatgcatgaggctctgcacaaccactacactcagaagtctctgtctctgtcccccggcaaatga

SEQ ID NO: 250 - DNA, 1392 bp, synthetic construct
  source 1..1392: mol_type = other DNA
atggaccctaagggcagcctcagctggaggattctgctgttcctctctctggcctttgaactgagctacggccgcaacctgccggtggcgacaccagatcccggcatgttcccttgcctccatcacagccagaatctgctgagggccgtcagcaatatgctgcagaaggctaggcagacactcgagttctatccttgcacaagcgaggagatcgaccatgaggatatcactaaggacaagactagcactgtggaggcttgtctcccactggaactgactaagaacgagagctgtctgaactctagggagacaagcttcatcactaacggcagctgtctcgcctctaggaagacttccttcatgatggctctgtgcctcagctccatctacgaagatctgaagatgtaccaagtggagtttaaaactatgaacgccaagctgctgatggaccctaagaggcagatctttctcgaccagaacatgctggctgtcatcgacgaactcatgcaagccctcaatttcaacagcgagactgtccctcagaagtcctctctggaggaaccagatttctacaagactaagatcaagctctgtattctgctgcacgcctttaggattagggctgtcacaatcgatagggtcatgagctatctgaatgccagcggcggaggaggcagcggaggaggaggaagcggaggcggcggaagcgacaagactcacacatgtccaccttgtccagccccagaggctgctggaggaccttccgtctttctgttcccacctaagcctaaggacacactcatgatctctaggacaccagaggtgacatgtgtggtcgtgtccgtcagccacgaggacccagaggtgaagtttaactggtacgtggacggcgtggaagtgcacaatgccaagacaaagccaagggaggagcaatacaacagcacttatagggtcgtgagcgtgctgacagtgctgcatcaagattggctgaacggcaaggagtacaagtgcaaggtgagcaacaaagctctgccagcccctatcgagaagactatcagcaaggccaagggccagcctagggagccacaagtgtacacactgcctccttgtagggacgagctgactaagaaccaagtgtctctgtggtgtctggtcaagggcttttacccttccgatatcgccgtggagtgggaatccaacggccagccagagaataactacaagacaactcctccagtgctggactccgatggctccttcttcctctacagcaagctgactgtggataagagcagatggcagcaaggcaacgtgttcagctgtagcgtgatgcatgaggctctgcacaaccactacactcagaagtctctgtctctgtcccccggcaaatga

SEQ ID NO: 251 - DNA, 573 bp, synthetic construct
  source 1..573: mol_type = other DNA
actccagatcccggcatgttcccttgtctgcaccacagccagaatctgctgagggctgtgagcaatatgctgcagaaggccagacagacactcgagttctacccttgcacaagcgaggagagcgaccacgaagacatcacaaaggataagactagcactgtcgaggcttgtctccctctggagctgacaaagaacgagagctgcctcaactctagggagacaagcttcatcactaacggcagctgtctggcttccagaaagactagcttcatgatggctctgtgtctgagcagcatctacgaggatctgaagatgtaccaagtggagttcaaaactatgaacgccaagctgctcatggaccctaagaggcagatctttctggaccagaacatgctggccgtcatcgacgagctgatgcaagccctcaatttcaacagcgagactgtccctcagaagtcctctctggaggagccagacttctataagactaaaatcaaactctgtattctgctgcacgccttcagaatcagagccgtgacaatcgatagggtgatgagctatctgaatgctagc

SEQ ID NO: 252 - DNA, 573 bp, synthetic construct
  source 1..573: mol_type = other DNA
actccagatcccggcatgttcccttgtctgcaccacagccagaatctgctgagggctgtgagcaatatgctgcagaaggccagacagacactcgagttctacccttgcacaagcgaggagatcgaccacagagacatcacaaaggataagactagcactgtcgaggcttgtctccctctggagctgacaaagaacgagagctgcctcaactctagggagacaagcttcatcactaacggcagctgtctggcttccagaaagactagcttcatgatggctctgtgtctgagcagcatctacgaggatctgaagatgtaccaagtggagttcaaaactatgaacgccaagctgctcatggaccctaagaggcagatctttctggaccagaacatgctggccgtcatcgacgagctgatgcaagccctcaatttcaacagcgagactgtccctcagaagtcctctctggaggagccagacttctataagactaaaatcaaactctgtattctgctgcacgccttcagaatcagagccgtgacaatcgatagggtgatgagctatctgaatgctagc

SEQ ID NO: 253 - DNA, 573 bp, synthetic construct
  source 1..573: mol_type = other DNA
actccagatcccggcatgttcccttgtctgcaccacagccagaatctgctgagggccgtgagcaatatgctgcagaaagctaggcagacactggagttctacccttgcactagcgaggagatcgatcacgaggatatcacaaaggacaagacatccactgtggaggcttgtctgccactggagctcactaagaacgagagctgtctgaactctagggaaacaagcttcatcactaatggcagctgtctggcttctaggaagacatccttcatgatggctctgtgtctgagctccatctacgaggatctgaagatgtaccaagtggagttcaagacaatgaacgccaagctgctgatggaccctaagagacagatctttctggaccagaacatgctggccgtcatcgatgaactgatgcaagctctgaacttcaacagcgagacagtgcctcagaagagctctctggaggagccaaagttctacaagacaaagattaaactgtgtattctgctgcacgcctttaggattagggctgtcactatcgatagggtgatgagctatctgaatgctagc

SEQ ID NO: 254 - DNA, 573 bp, synthetic construct
  source 1..573: mol_type = other DNA
actccagatcccggcatgttcccttgtctgcaccacagccagaatctgctgagggccgtgagcaatatgctgcagaaagctaggcagacactggagttctacccttgcactagcgaggagatcgatcacgaggatatcacaaaggacaagacatccactgtggaggcttgtctgccactggagctcactaagaacgagagctgtctgaactctagggaaacaagcttcatcactaatggcagctgtctggcttctaggaagacatccttcatgatggctctgtgtctgagctccatctacgaggatctgaagatgtaccaagtggagttcaagacaatgaacgccaagctgctgatggaccctaagagacagatctttctggaccagaacatgctggccgtcatcgatgaactgatgcaagctctgaacttcaacagcgagacagtgcctcagaagagctctctggaggagccaaggttctacaagacaaagattaaactgtgtattctgctgcacgcctttaggattagggctgtcactatcgatagggtgatgagctatctgaatgctagc

SEQ ID NO: 255 - DNA, 573 bp, synthetic construct
  source 1..573: mol_type = other DNA
actccagatcccggcatgttcccttgtctgcaccacagccagaatctgctgagggccgtgagcaatatgctgcagaaagctaggcagacactggagttctacccttgcactagcgaggagatcgatcacgaggatatcacaaaggacaagacatccactgtggaggcttgtctgccactggagctcactaagaacgagagctgtctgaactctagggaaacaagcttcatcactaatggcagctgtctggcttctaggaagacatccttcatgatggctctgtgtctgagctccatctacgaggatctgaagatgtaccaagtggagttcaagacaatgaacgccaagctgctgatggaccctaagagacagatctttctggaccagaacatgctggccgtcatcgatgaactgatgcaagctctgaacttcaacagcgagacagtgcctcagaagagctctctggaggagccagacttcagcaagacaaagattaaactgtgtattctgctgcacgcctttaggattagggctgtcactatcgatagggtgatgagctatctgaatgctagc

SEQ ID NO: 256 - DNA, 573 bp, synthetic construct
  source 1..573: mol_type = other DNA
actccagatcccggcatgttcccttgtctgcaccacagccagaatctgctgagggccgtgagcaatatgctgcagaaagctaggcagacactggagttctacccttgcactagcgaggagatcgatcacgaggatatcacaaaggacaagacatccactgtggaggcttgtctgccactggagctcactaagaacgagagctgtctgaactctagggaaacaagcttcatcactaatggcagctgtctggcttctaggaagacatccttcatgatggctctgtgtctgagctccatctacgaggatctgaagatgtaccaagtggagttcaagacaatgaacgccaagctgctgatggaccctaagagacagatctttctggaccagaacatgctggccgtcatcgatgaactgatgcaagctctgaacttcaacagcgagacagtgcctcagaagagctctctggaggagccagacttcactaagacaaagattaaactgtgtattctgctgcacgcctttaggattagggctgtcactatcgatagggtgatgagctatctgaatgctagc

SEQ ID NO: 257 - DNA, 573 bp, synthetic construct
  source 1..573: mol_type = other DNA
actccagatcccggcatgttcccttgtctgcaccacagccagaatctgctgagggccgtgagcaatatgctgcagaaagctaggcagacactggagttctacccttgcactagcgaggagatcgatcacgaggatatcacaaaggacaagacatccactgtggaggcttgtctgccactggagctcactaagaacgagagctgtctgaactctagggaaacaagcttcatcactaatggcagctgtctggcttctaggaagacatccttcatgatggctctgtgtctgagctccatctacgaggatctgaagatgtaccaagtggagttcaagacaatgaacgccaagctgctgatggaccctaagagacagatctttctggaccagaacatgctggccgtcatcgatgaactgatgcaagctctgaacttcaacagcgagacagtgcctcagaagagctctctggaggagccagacttcgctaagacaaagattaaactgtgtattctgctgcacgcctttaggattagggctgtcactatcgatagggtgatgagctatctgaatgctagc

SEQ ID NO: 258 - DNA, 918 bp, synthetic construct
  source 1..918: mol_type = other DNA
atttgggagctgaagaaggacgtgtacgtggtggagctggattggtatccagatgcccccggcgagatggtggtgctgacatgtgacactccagaggaagacggcatcacttggactctcgatcagtcctccgaagtgctgggctccggaaagacactgacaatccaagtgaaggagttcggagatgctggccagtacacttgccacaaaggcggcgaagtgctgagccactctctgctgctgctgcataagaaggagctgggaatctggtccacagacatcctcaaggaccaaaaggagccaaaaaacaagacatttctgaggtgtgaggccaagaactactccggaaggtttacatgctggtggctcacaacaatctccacagacctcactttcagcgtgaagagcagcagaggcagcagcgacccacaaggcgtgacttgtggagccgccactctgtccgctgagagggtgaggggcgacaacaaggagtacgagtactccgtggagtgccaagaggatagcgcttgtccagccgctgaggaaagcctccctatcgaggtgatggtggacgccgtgcacaagctgaagtatgaaaattacacatccagcttcttcattagggacatcatcaagccagatcctccaaagaacctccagctcaagcctctgaagaactctaggcaagtggaggtcagctgggagtatccagacacttggagcacacctcactcctacttctctctgacattctgcgtccaagtgcaaggcaagtccaagagagagaaaaaggatagggtgttcactgacaagactagcgccacagtgatctgcagaaagaacgccagcatcagcgtgagagcccaagataggtactattcctccagctggagcgagtgggcttccgtgccttgtagc

SEQ ID NO: 259 - DNA, 918 bp, synthetic construct
  source 1..918: mol_type = other DNA
atctgggaactgaagaaggacgtctacgtcgtggagctggattggtatccagatgctcccggcgaaatggtggtgctcacatgcgatactccagaagaggacggcatcacatggacactggatcagagcagcgaagtgctgggctccggaaagacactgactatccaagtgaaggagtttggcgatgctggccagtacacatgtcacaagggcggagaggtgctgagccactctctgctgctgctgcacaagaaggaggatggcatctggtccacagacattctgaaggaccagaaggagcctaagaacgagacatttctgaggtgcgaagccaagaattattccggaaggtttacatgctggtggctgacaacaatcagcacagacctcactttcagcgtgaagtcctctaggggcagcagcgaccctcaaggagtcacatgcggcgccgctactctgtccgccgaaagggtgaggggcgacaacaaagagtacgagtacagcgtggagtgtcaagaggatagcgcttgtccagccgctgaagagtctctgccaatcgaggtcatggtcgatgccgtgcacaagctcaagtacgaaaactacactagctccttcttcatcagagatatcatcaagccagatccaccaaagaacctccagctgaagcctctgaagaactctaggcaagtggaggtgagctgggagtatccagatacttggtccactcctcacagctacttctctctgactttctgtgtccaagtgcaaggcaagtccaagagagagaagaaggatagggtcttcacagacaagacaagcgctactgtgatctgtaggaagaacgcctccatcagcgtgagggcccaagataggtactacagctccagctggagcgagtgggcttccgtgccatgtagc

SEQ ID NO: 260 - DNA, 918 bp, synthetic construct
  source 1..918: mol_type = other DNA
atctgggaactgaagaaggacgtctacgtcgtggagctggattggtatccagatgctcccggcgaaatggtggtgctcacatgcgatactccagaagaggacggcatcacatggacactggatcagagcagcgaagtgctgggctccggaaagacactgactatccaagtgaaggagtttggcgatgctggccagtacacatgtcacaagggcggagaggtgctgagccactctctgctgctgctgcacaagaaggaggatggcatctggtccacagacattctgaaggaccagaaggagcctaagaacgacacatttctgaggtgcgaagccaagaattattccggaaggtttacatgctggtggctgacaacaatcagcacagacctcactttcagcgtgaagtcctctaggggcagcagcgaccctcaaggagtcacatgcggcgccgctactctgtccgccgaaagggtgaggggcgacaacaaagagtacgagtacagcgtggagtgtcaagaggatagcgcttgtccagccgctgaagagtctctgccaatcgaggtcatggtcgatgccgtgcacaagctcaagtacgaaaactacactagctccttcttcatcagagatatcatcaagccagatccaccaaagaacctccagctgaagcctctgaagaactctaggcaagtggaggtgagctgggagtatccagatacttggtccactcctcacagctacttctctctgactttctgtgtccaagtgcaaggcaagtccaagagagagaagaaggatagggtcttcacagacaagacaagcgctactgtgatctgtaggaagaacgcctccatcagcgtgagggcccaagataggtactacagctccagctggagcgagtgggcttccgtgccatgtagc

SEQ ID NO: 261 - protein, 197 aa, synthetic construct
RNLPVATPDPGMFPCLHHSQNLLRAVSNMLQKARQTLEFYPCTSEKIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 262 - protein, 197 aa, synthetic construct
RNLPVATPDPGMFPCLHHSQNLLRAVSNMLQKARQTLEFYPCTSERIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 263 - protein, 197 aa, synthetic construct
RNLPVATPDPGMFPCLHHSQNLLRAVSNMLQKARQTLEFYPCTSEWIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 264 - protein, 197 aa, synthetic construct
RNLPVATPDPGMFPCLHHSQNLLRAVSNMLQKARQTLEFYPCTSEEADHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 265 - protein, 197 aa, synthetic construct
RNLPVATPDPGMFPCLHHSQNLLRAVSNMLQKARQTLEFYPCTSEEVDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 266 - protein, 197 aa, synthetic construct
RNLPVATPDPGMFPCLHHSQNLLRAVSNMLQKARQTLEFYPCTSEESDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 267 - protein, 197 aa, synthetic construct
RNLPVATPDPGMFPCLHHSQNLLRAVSNMLQKARQTLEFYPCTSEETDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 268 - protein, 197 aa, synthetic construct
RNLPVATPDPGMFPCLHHSQNLLRAVSNMLQKARQTLEFYPCTSEEIAHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 269 - protein, 197 aa, synthetic construct
RNLPVATPDPGMFPCLHHSQNLLRAVSNMLQKARQTLEFYPCTSEEILHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 270 - protein, 197 aa, synthetic construct
RNLPVATPDPGMFPCLHHSQNLLRAVSNMLQKARQTLEFYPCTSEEIWHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 271 - protein, 197 aa, synthetic construct
RNLPVATPDPGMFPCLHHSQNLLRAVSNMLQKARQTLEFYPCTSEEIDHADITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 272 - protein, 197 aa, synthetic construct
RNLPVATPDPGMFPCLHHSQNLLRAVSNMLQKARQTLEFYPCTSEEIDHKDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 273 - protein, 197 aa, synthetic construct
RNLPVATPDPGMFPCLHHSQNLLRAVSNMLQKARQTLEFYPCTSEEIDHRDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 274 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFYKTKIKLCILLHAFRIRAVTIDRVMSYLNAS

SEQ ID NO: 275 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPRFYKTKIKLCILLHAFRIRAVTIDRVMSYLNAS

SEQ ID NO: 276 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPYFYKTKIKLCILLHAFRIRAVTIDRVMSYLNAS

SEQ ID NO: 277 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPFFYKTKIKLCILLHAFRIRAVTIDRVMSYLNAS

SEQ ID NO: 278 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFSKTKIKLCILLHAFRIRAVTIDRVMSYLNAS

SEQ ID NO: 279 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNAS

SEQ ID NO: 280 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFAKTKIKLCILLHAFRIRAVTIDRVMSYLNAS

SEQ ID NO: 281 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFWKTKIKLCILLHAFRIRAVTIDRVMSYLNAS

SEQ ID NO: 282 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYETKIKLCILLHAFRIRAVTIDRVMSYLNAS

SEQ ID NO: 283 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYDTKIKLCILLHAFRIRAVTIDRVMSYLNAS

SEQ ID NO: 284 - protein, 197 aa, synthetic construct
RNLPVATPDPGMFPCLHHSQNLLRAVSNMLQKARQTLEGYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNAS

SEQ ID NO: 285 - protein, 197 aa, synthetic construct
RNLPVATPDPGMFPCLHHSQNLLRAVSNMLQKARQTLEKYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNAS

SEQ ID NO: 286 - protein, 197 aa, synthetic construct
RNLPVATPDPGMFPCLHHSQNLLRAVSNMLQKARQTLESYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNAS

SEQ ID NO: 287 - protein, 197 aa, synthetic construct
RNLPVATPDPGMFPCLHHSQNLLRAVSNMLQKARQTLEFGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNAS

SEQ ID NO: 288 - protein, 197 aa, synthetic construct
RNLPVATPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNAS

SEQ ID NO: 289 - protein, 197 aa, synthetic construct
RNLPVATPDPGMFPCLHHSQNLLRAVSNMLQKARQTLEFTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNAS

SEQ ID NO: 290 - protein, 197 aa, synthetic construct
RNLPVATPDPGMFPCLHHSQNLLRAVSNMLQKARQTLEFYQCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNAS

SEQ ID NO: 291 - protein, 197 aa, synthetic construct
RNLPVATPDPGMFPCLHHSQNLLRAVSNMLQKARQTLEFYECTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNAS

SEQ ID NO: 292 - protein, 197 aa, synthetic construct
RNLPVATPDPGMFPCLHHSQNLLRAVSNMLQKARQTLESTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNAS

SEQ ID NO: 293 - protein, 197 aa, synthetic construct
RNLPVATPDPGMFPCLHHSQNLLRAVSNMLQKARQTLESSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNAS

SEQ ID NO: 294 - protein, 197 aa, synthetic construct
RNLPVATPDPGMFPCLHHSQNLLRAVSNMLQKARQTLEGYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNAS

SEQ ID NO: 295 - protein, 197 aa, synthetic construct
RNLPVATPDPGMFPCLHHSQNLLRAVSNMLQKARQTLEKYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNAS

SEQ ID NO: 296 - protein, 197 aa, synthetic construct
RNLPVATPDPGMFPCLHHSQNLLRAVSNMLQKARQTLESYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNAS

SEQ ID NO: 297 - protein, 197 aa, synthetic construct
RNLPVATPDPGMFPCLHHSQNLLRAVSNMLQKARQTLEFGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNAS

SEQ ID NO: 298 - protein, 197 aa, synthetic construct
RNLPVATPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNAS

SEQ ID NO: 299 - protein, 197 aa, synthetic construct
RNLPVATPDPGMFPCLHHSQNLLRAVSNMLQKARQTLEFTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNAS

SEQ ID NO: 300 - protein, 198 aa, synthetic construct
RNLPVATPDPGMFPCLHHSQNLLRAVSNMLQKARQTLESTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASG

SEQ ID NO: 301 - protein, 197 aa, synthetic construct
RNLPVATPDPGMFPCLHHSQNLLRAVSNMLQKARQTLESSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNAS

SEQ ID NO: 302 - protein, 197 aa, synthetic construct
RNLPVATPDPGMFPCLHHSQNLLRAVSNMLQKARQTLESGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNAS

SEQ ID NO: 303 - protein, 197 aa, synthetic construct; identical to SEQ ID NO: 284

SEQ ID NO: 304 - protein, 439 aa, synthetic construct
RNLPVATPDPGMFPCLHHSQNLLRAVSNMLQKARQTLEFYPCTSEKIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 305 - protein, 439 aa, synthetic construct
RNLPVATPDPGMFPCLHHSQNLLRAVSNMLQKARQTLEFYPCTSERIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 306 - protein, 439 aa, synthetic construct
RNLPVATPDPGMFPCLHHSQNLLRAVSNMLQKARQTLEFYPCTSEWIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 307 - protein, 439 aa, synthetic construct
RNLPVATPDPGMFPCLHHSQNLLRAVSNMLQKARQTLEFYPCTSEEADHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 308 - protein, 439 aa, synthetic construct
RNLPVATPDPGMFPCLHHSQNLLRAVSNMLQKARQTLEFYPCTSEEVDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 309 - protein, 439 aa, synthetic construct
RNLPVATPDPGMFPCLHHSQNLLRAVSNMLQKARQTLEFYPCTSEESDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 310 - protein, 439 aa, synthetic construct
RNLPVATPDPGMFPCLHHSQNLLRAVSNMLQKARQTLEFYPCTSEETDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 311 - protein, 439 aa, synthetic construct
RNLPVATPDPGMFPCLHHSQNLLRAVSNMLQKARQTLEFYPCTSEEIAHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 312 - protein, 439 aa, synthetic construct
RNLPVATPDPGMFPCLHHSQNLLRAVSNMLQKARQTLEFYPCTSEEILHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 313 - protein, 439 aa, synthetic construct
RNLPVATPDPGMFPCLHHSQNLLRAVSNMLQKARQTLEFYPCTSEEIWHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 314 - protein, 439 aa, synthetic construct
RNLPVATPDPGMFPCLHHSQNLLRAVSNMLQKARQTLEFYPCTSEEIDHADITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 315 - protein, 439 aa, synthetic construct
RNLPVATPDPGMFPCLHHSQNLLRAVSNMLQKARQTLEFYPCTSEEIDHKDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 316 - protein, 439 aa, synthetic construct
RNLPVATPDPGMFPCLHHSQNLLRAVSNMLQKARQTLEFYPCTSEEIDHRDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 317 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 318 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPRFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 319 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPYFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 320 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPFFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 321 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFSKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 323 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFAKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 324 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFWKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 325 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYETKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 326 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYDTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 327 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 328 - protein, 439 aa, synthetic construct
RNLPVATPDPGMFPCLHHSQNLLRAVSNMLQKARQTLEGYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 329 - protein, 439 aa, synthetic construct
RNLPVATPDPGMFPCLHHSQNLLRAVSNMLQKARQTLEKYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 330 - protein, 439 aa, synthetic construct
RNLPVATPDPGMFPCLHHSQNLLRAVSNMLQKARQTLESYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 331 - protein, 439 aa, synthetic construct
RNLPVATPDPGMFPCLHHSQNLLRAVSNMLQKARQTLEFGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 332 - protein, 439 aa, synthetic construct
RNLPVATPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 333 - protein, 439 aa, synthetic construct
RNLPVATPDPGMFPCLHHSQNLLRAVSNMLQKARQTLEFTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 334 - protein, 439 aa, synthetic construct
RNLPVATPDPGMFPCLHHSQNLLRAVSNMLQKARQTLEFYQCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 335 - protein, 439 aa, synthetic construct
RNLPVATPDPGMFPCLHHSQNLLRAVSNMLQKARQTLEFYECTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 336 - protein, 439 aa, synthetic construct
RNLPVATPDPGMFPCLHHSQNLLRAVSNMLQKARQTLESTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 337 - protein, 439 aa, synthetic construct
RNLPVATPDPGMFPCLHHSQNLLRAVSNMLQKARQTLESSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 338 - protein, 439 aa, synthetic construct
RNLPVATPDPGMFPCLHHSQNLLRAVSNMLQKARQTLEGYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 339 - protein, 439 aa, synthetic construct
RNLPVATPDPGMFPCLHHSQNLLRAVSNMLQKARQTLEKYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 340 - protein, 439 aa, synthetic construct
RNLPVATPDPGMFPCLHHSQNLLRAVSNMLQKARQTLESYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 341 - protein, 439 aa, synthetic construct
RNLPVATPDPGMFPCLHHSQNLLRAVSNMLQKARQTLEFGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 342 - protein, 439 aa, synthetic construct
RNLPVATPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 343 - protein, 439 aa, synthetic construct
RNLPVATPDPGMFPCLHHSQNLLRAVSNMLQKARQTLEFTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 344 - protein, 440 aa, synthetic construct
RNLPVATPDPGMFPCLHHSQNLLRAVSNMLQKARQTLESTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 345 - protein, 439 aa, synthetic construct
RNLPVATPDPGMFPCLHHSQNLLRAVSNMLQKARQTLESSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 346 - protein, 439 aa, synthetic construct
RNLPVATPDPGMFPCLHHSQNLLRAVSNMLQKARQTLESGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 347 - protein, 439 aa, synthetic construct
RNLPVATPDPGMFPCLHHSQNLLRAVSNMLQKARQTLEFYPCTSEKIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 348 - protein, 439 aa, synthetic construct
RNLPVATPDPGMFPCLHHSQNLLRAVSNMLQKARQTLEFYPCTSERIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 349 - protein, 439 aa, synthetic construct
RNLPVATPDPGMFPCLHHSQNLLRAVSNMLQKARQTLEFYPCTSEWIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 350 - protein, 439 aa, synthetic construct
RNLPVATPDPGMFPCLHHSQNLLRAVSNMLQKARQTLEFYPCTSEEADHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 351 - protein, 439 aa, synthetic construct
RNLPVATPDPGMFPCLHHSQNLLRAVSNMLQKARQTLEFYPCTSEEVDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 352 - protein, 439 aa, synthetic construct
RNLPVATPDPGMFPCLHHSQNLLRAVSNMLQKARQTLEFYPCTSEESDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 353 - protein, 439 aa, synthetic construct
RNLPVATPDPGMFPCLHHSQNLLRAVSNMLQKARQTLEFYPCTSEETDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 354 - protein, 439 aa, synthetic construct
RNLPVATPDPGMFPCLHHSQNLLRAVSNMLQKARQTLEFYPCTSEEIAHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 355 - protein, 439 aa, synthetic construct
RNLPVATPDPGMFPCLHHSQNLLRAVSNMLQKARQTLEFYPCTSEEILHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 356 - protein, 439 aa, synthetic construct
RNLPVATPDPGMFPCLHHSQNLLRAVSNMLQKARQTLEFYPCTSEEIWHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 357 - protein, 439 aa, synthetic construct
RNLPVATPDPGMFPCLHHSQNLLRAVSNMLQKARQTLEFYPCTSEEIDHADITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 358 - protein, 439 aa, synthetic construct
RNLPVATPDPGMFPCLHHSQNLLRAVSNMLQKARQTLEFYPCTSEEIDHKDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 359 - protein, 439 aa, synthetic construct
RNLPVATPDPGMFPCLHHSQNLLRAVSNMLQKARQTLEFYPCTSEEIDHRDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 360 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 361 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPRFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 362 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPYFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 363 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPFFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 364 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFSKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 365 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 366 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFAKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 367 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFWKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 368 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYETKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 369 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYDTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 370 - protein, 439 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 371 - protein, 439 aa, synthetic construct
RNLPVATPDPGMFPCLHHSQNLLRAVSNMLQKARQTLEGYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 372 - protein, 439 aa, synthetic construct
RNLPVATPDPGMFPCLHHSQNLLRAVSNMLQKARQTLEKYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 373 - protein, 439 aa, synthetic construct; identical to SEQ ID NO: 330

SEQ ID NO: 374 - protein, 439 aa, synthetic construct
RNLPVATPDPGMFPCLHHSQNLLRAVSNMLQKARQTLEFGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 375 - protein, 439 aa, synthetic construct
RNLPVATPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 376 - protein, 439 aa, synthetic construct
RNLPVATPDPGMFPCLHHSQNLLRAVSNMLQKARQTLEFTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 377 - protein, 439 aa, synthetic construct
RNLPVATPDPGMFPCLHHSQNLLRAVSNMLQKARQTLEFYQCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 378 - protein, 439 aa, synthetic construct
RNLPVATPDPGMFPCLHHSQNLLRAVSNMLQKARQTLEFYECTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 379 - protein, 439 aa, synthetic construct
RNLPVATPDPGMFPCLHHSQNLLRAVSNMLQKARQTLESTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 380 - protein, 439 aa, synthetic construct
RNLPVATPDPGMFPCLHHSQNLLRAVSNMLQKARQTLESSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPKFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 381 - protein, 439 aa, synthetic construct
RNLPVATPDPGMFPCLHHSQNLLRAVSNMLQKARQTLEGYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 382 - protein, 439 aa, synthetic construct
RNLPVATPDPGMFPCLHHSQNLLRAVSNMLQKARQTLEKYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 383 - protein, 439 aa, synthetic construct
RNLPVATPDPGMFPCLHHSQNLLRAVSNMLQKARQTLESYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 384 - protein, 439 aa, synthetic construct
RNLPVATPDPGMFPCLHHSQNLLRAVSNMLQKARQTLEFGPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 385 - protein, 439 aa, synthetic construct
RNLPVATPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 386 - protein, 439 aa, synthetic construct
RNLPVATPDPGMFPCLHHSQNLLRAVSNMLQKARQTLEFTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 387 - protein, 439 aa, synthetic construct
RNLPVATPDPGMFPCLHHSQNLLRAVSNMLQKARQTLESTPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFTKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 388 - protein, 439 aa, synthetic construct
RNLVPATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 389 - protein, 439 aa, synthetic construct
RNLVPATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 390 - protein, 4 aa, synthetic construct
RNLP

SEQ ID NO: 391 - protein, 5 aa, synthetic construct
RNLPV

SEQ ID NO: 392 - protein, 7 aa, synthetic construct
RNLPVAT

SEQ ID NO: 393 - protein, 8 aa, synthetic construct
RNLPVATP

SEQ ID NO: 394 - protein, 9 aa, synthetic construct
RNLPVATPD

SEQ ID NO: 395 - protein, 10 aa, synthetic construct
RNLPVATPDP

SEQ ID NO: 396 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 397 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 398 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSWLTVDKSRWQQGNVFSCSVLHEALHSHYTQKSLSLSPGK

SEQ ID NO: 399 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLVSDGSFTLYSKLTVDKSRWQQGNVFSCSVLHEALHSHYTQKSLSLSPGK

SEQ ID NO: 400 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDWLTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 401 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVSTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 402 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVDHHDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHSHYTQKSLSLSPGK

SEQ ID NO: 403 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVDHHDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHSHYTQKSLSLSPGK

SEQ ID NO: 404 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSWLTVDKSRWQQGNVFSCSVMHEALHSHYTQKSLSLSPGK

SEQ ID NO: 405 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLVSDGSFTLYSKLTVDKSRWQQGNVFSCSVMHEALHSHYTQKSLSLSPGK

SEQ ID NO: 406 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVDHHDWLNGKEYKCKVSNKALPAPIEKTISKAKGQPREPQVYTLPPSRDWLTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 407 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVDHHDWLNGKEYKCKVSNKALPAPIEKTISKAKGQPREPQVSTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 408 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALKFHYTQKSLSLSPGK

SEQ ID NO: 409 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALKFHYTQKSLSLSPGK

SEQ ID NO: 410 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSWLTVDKSRWQQGNVFSCSVMHEALKFHYTQKSLSLSPGK

SEQ ID NO: 411 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLVSDGSFTLYSKLTVDKSRWQQGNVFSCSVMHEALKFHYTQKSLSLSPGK

SEQ ID NO: 412 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDWLTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 413 - protein, 227 aa, synthetic construct
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVSTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK